SEQUENCE LISTING (452 sequences)

SEQ ID NO: 1 - protein, 4 aa, Artificial Sequence
  Description of Artificial Sequence Synthetic peptide
HHHH

SEQ ID NO: 2 - protein, 4 aa, Artificial Sequence
  Description of Artificial Sequence Synthetic peptide
KHKK

SEQ ID NO: 3 - protein, 4 aa, Artificial Sequence
  Description of Artificial Sequence Synthetic peptide
KKHK

SEQ ID NO: 4 - protein, 4 aa, Artificial Sequence
  Description of Artificial Sequence Synthetic peptide
KKKH

SEQ ID NO: 5 - protein, 4 aa, Artificial Sequence
  Description of Artificial Sequence Synthetic peptide
KHKH

SEQ ID NO: 6 - protein, 4 aa, Artificial Sequence
  Description of Artificial Sequence Synthetic peptide
HKHK

SEQ ID NO: 7 - protein, 4 aa, Artificial Sequence
  Description of Artificial Sequence Synthetic peptide
KKKK

SEQ ID NO: 8 - protein, 4 aa, Artificial Sequence
  Description of Artificial Sequence Synthetic peptide
KKRK

SEQ ID NO: 9 - protein, 4 aa, Artificial Sequence
  Description of Artificial Sequence Synthetic peptide
KRKK

SEQ ID NO: 10 - protein, 4 aa, Artificial Sequence
  Description of Artificial Sequence Synthetic peptide
KRRK

SEQ ID NO: 11 - protein, 4 aa, Artificial Sequence
  Description of Artificial Sequence Synthetic peptide
RKKR

SEQ ID NO: 12 - protein, 4 aa, Artificial Sequence
  Description of Artificial Sequence Synthetic peptide
RRRR

SEQ ID NO: 13 - protein, 4 aa, Artificial Sequence
  Description of Artificial Sequence Synthetic peptide
KGKK

SEQ ID NO: 14 - protein, 4 aa, Artificial Sequence
  Description of Artificial Sequence Synthetic peptide
KKGK

SEQ ID NO: 15 - protein, 5 aa, Artificial Sequence
  Description of Artificial Sequence Synthetic peptide
  MOD_RES (2)..(2): Beta-alanine
  MOD_RES (4)..(4): Beta-alanine
HAHAH

SEQ ID NO: 16 - protein, 5 aa, Artificial Sequence
  Description of Artificial Sequence Synthetic peptide
  MOD_RES (2)..(2): Beta-alanine
  MOD_RES (4)..(4): Beta-alanine
HAKAH

SEQ ID NO: 17 - protein, 5 aa, Artificial Sequence
  Description of Artificial Sequence Synthetic peptide
RRRRR

SEQ ID NO: 18 - protein, 5 aa, Artificial Sequence
  Description of Artificial Sequence Synthetic peptide
KKKKK

SEQ ID NO: 19 - protein, 5 aa, Artificial Sequence
  Description of Artificial Sequence Synthetic peptide
KKKRK

SEQ ID NO: 20 - protein, 5 aa, Artificial Sequence
  Description of Artificial Sequence Synthetic peptide
RKKKK

SEQ ID NO: 21 - protein, 5 aa, Artificial Sequence
  Description of Artificial Sequence Synthetic peptide
KRKKK

SEQ ID NO: 22 - protein, 5 aa, Artificial Sequence
  Description of Artificial Sequence Synthetic peptide
KKRKK

SEQ ID NO: 23 - protein, 5 aa, Artificial Sequence
  Description of Artificial Sequence Synthetic peptide
KKKKR

SEQ ID NO: 24 - protein, 5 aa, Artificial Sequence
  Description of Artificial Sequence Synthetic peptide
  MOD_RES (2)..(2): Beta-alanine
  MOD_RES (4)..(4): Beta-alanine
KAKAK

SEQ ID NO: 25 - protein, 6 aa, Artificial Sequence
  Description of Artificial Sequence Synthetic peptide
RKKKKG

SEQ ID NO: 26 - protein, 6 aa, Artificial Sequence
  Description of Artificial Sequence Synthetic peptide
KRKKKG

SEQ ID NO: 27 - protein, 6 aa, Artificial Sequence
  Description of Artificial Sequence Synthetic peptide
KKRKKG

SEQ ID NO: 28 - protein, 6 aa, Artificial Sequence
  Description of Artificial Sequence Synthetic peptide
KKKKRG

SEQ ID NO: 29 - protein, 6 aa, Artificial Sequence
  Description of Artificial Sequence Synthetic peptide
  MOD_RES (6)..(6): Beta-alanine
RKKKKA

SEQ ID NO: 30 - protein, 6 aa, Artificial Sequence
  Description of Artificial Sequence Synthetic peptide
  MOD_RES (6)..(6): Beta-alanine
KRKKKA

SEQ ID NO: 31 - protein, 6 aa, Artificial Sequence
  Description of Artificial Sequence Synthetic peptide
  MOD_RES (6)..(6): Beta-alanine
KKRKKA

SEQ ID NO: 32 - protein, 6 aa, Artificial Sequence
  Description of Artificial Sequence Synthetic peptide
  MOD_RES (6)..(6): Beta-alanine
KKKKRA

SEQ ID NO: 33 - protein, 6 aa, Artificial Sequence
  Description of Artificial Sequence Synthetic peptide
KKKRKV

SEQ ID NO: 34 - protein, 6 aa, Artificial Sequence
  Description of Artificial Sequence Synthetic peptide
RRRRRR

SEQ ID NO: 35 - protein, 6 aa, Artificial Sequence
  Description of Artificial Sequence Synthetic peptide
HHHHHH

SEQ ID NO: 36 - protein, 6 aa, Artificial Sequence
  Description of Artificial Sequence Synthetic peptide
RHRHRH

SEQ ID NO: 37 - protein, 6 aa, Artificial Sequence
  Description of Artificial Sequence Synthetic peptide
HRHRHR

SEQ ID NO: 38 - protein, 6 aa, Artificial Sequence
  Description of Artificial Sequence Synthetic peptide
KRKRKR

SEQ ID NO: 39 - protein, 6 aa, Artificial Sequence
  Description of Artificial Sequence Synthetic peptide
RKRKRK

SEQ ID NO: 40 - protein, 6 aa, Artificial Sequence
  Description of Artificial Sequence Synthetic peptide
  MOD_RES (2)..(2): Beta-alanine
  MOD_RES (4)..(4): Beta-alanine
  MOD_RES (6)..(6): Beta-alanine
RARARA

SEQ ID NO: 41 - protein, 6 aa, Artificial Sequence
  Description of Artificial Sequence Synthetic peptide
  MOD_RES (2)..(2): Beta-alanine
  MOD_RES (4)..(4): Beta-alanine
  MOD_RES (6)..(6): Beta-alanine
KAKAKA

SEQ ID NO: 42 - protein, 7 aa, Artificial Sequence
  Description of Artificial Sequence Synthetic peptide
PKKKRKV

SEQ ID NO: 43 - protein, 7 aa, Artificial Sequence
  Description of Artificial Sequence Synthetic peptide
PGKKRKV

SEQ ID NO: 44 - protein, 7 aa, Artificial Sequence
  Description of Artificial Sequence Synthetic peptide
PKGKRKV

SEQ ID NO: 45 - protein, 7 aa, Artificial Sequence
  Description of Artificial Sequence Synthetic peptide
PKKGRKV

SEQ ID NO: 46 - protein, 7 aa, Artificial Sequence
  Description of Artificial Sequence Synthetic peptide
PKKKGKV

SEQ ID NO: 47 - protein, 7 aa, Artificial Sequence
  Description of Artificial Sequence Synthetic peptide
PKKKRGV

SEQ ID NO: 48 - protein, 7 aa, Artificial Sequence
  Description of Artificial Sequence Synthetic peptide
PKKKRKG

SEQ ID NO: 49 - protein, 188 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCII

SEQ ID NO: 50 - protein, 188 aa, Artificial Sequence
  Description of Artificial Sequence Synthetic polypeptide
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCII

SEQ ID NO: 51 - protein, 188 aa, Artificial Sequence
  Description of Artificial Sequence Synthetic polypeptide
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCII

SEQ ID NO: 52 - protein, 185 aa, Artificial Sequence
  Description of Artificial Sequence Synthetic polypeptide
MTEYKLVVVGAGDVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKK

SEQ ID NO: 53 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 54 - protein, 189 aa, Artificial Sequence [sequence omitted]

SEQ ID NO: 55 - protein, 189 aa, Artificial Sequence
  Description of Artificial Sequence Synthetic polypeptide
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 56 - protein, 110 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVV

SEQ ID NO: 57 - protein, 178 aa, Homo sapiens
MTSFSTSAQCSTSDSACRISPGQINQVRPKLPLLKILHAAGAQGEMFTVKEVMHYLGQYIMVKQLYDQQEQHMVYCGGDLLGELLGRQSFSVKDPSPLYDMLRKNLVTLATATTDAAQTLALAQDHSMDIPSQDQLKQSAEESSTSRKRTTEDDIPTLPTSEHKCIHSREDEDLIENL

SEQ ID NO: 58 - protein, 149 aa, Homo sapiens
MEHIQGAWKTISNGFGFKDAVFDGSSCISPTIVQQFGYQRRASDDGKLTDPSKTSNTIRVFLPNKQRTVVNVRNGMSLHDCLMKALKVRGLQPECCAVFRLLHEHKGKKARLDWNTDAASLIGEELQVDFLDHVPLTTHNFARKTFLKL

SEQ ID NO: 59 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 60 - protein, 1036 aa, Homo sapiens
MKMASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLWLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 61 - protein, 1033 aa, Mus sp.
MTSVRCKLAQYLEDLEDVDLKKFKMHLEDYPPEKGCIPVPRGQMEKADHLDLATLMIDFNGEEKAWAMAVWIFAAINRRDLWEKAKKDQPEWNDTCTSHSSMVCQEDSLEEEWMGLLGYLSRISICKKKKDYCKMYRRHVRSRFYSIKDRNARLGESVDLNSRYTQLQLVKEHPSKQEREHELLTIGRTKMRDSPMSSLKLELLFEPEDGHSEPVHTVVFQGAAGIGKTILARKIMLDWALGKLFKDKFDYLFFIHCREVSLRTPRSLADLIVSCWPDPNPPVCKILRKPSRILFLMDGFDELQGAFDEHIGEVCTDWQKAVRGDILLSSLIRKKLLPKASLLITTRPVALEKLQHLLDHPRHVEILGFSEAKRKEYFFKYFSNELQAREAFRLIQENEVLFTMCFIPLVCWIVCTGLKQQMETGKSLAQTSKTTTAVYVFFLSSLLQSRGGIEEHLFSDYLQGLCSLAADGIWNQKILFEECDLRKHGLQKTDVSAFLRMNVFQKEVDCERFYSFSHMTFQEFFAAMYYLLEEEAEGETVRKGPGGCSDLLNRDVKVLLENYGKFEKGYLIFVVRFLFGLVNQERTSYLEKKLSCKISQQVRLELLKWIEVKAKAKKLQWQPSQLELFYCLYEMQEEDFVQSAMDHFPKIEINLSTRMDHVVSSFCIKNCHRVKTLSLGFFHNSPKEEEEERRGGRPLDQVQCVFPDTHVACSSRLVNCCLTSSFCRGLFSSLSTNRSLTELDLSDNTLGDPGMRVLCEALQHPGCNIQRLWLGRCGLSHQCCFDISSVLSSSQKLVELDLSDNALGDFGIRLLCVGLKHLLCNLQKLWLVSCCLTSACCQDLALVLSSNHSLTRLYIGENALGDSGVQVLCEKMKDPQCNLQKLGLVNSGLTSICCSALTSVLKTNQNFTHLYLRSNALGDTGLRLLCEGLLHPDCKLQMLELDNCSLTSHSCWNLSTILTHNHSLRKLNLGNNDLGDLCVVTLCEVLKQQGCLLQSLQLGEMYLNRETKRALEALQEEKPELTIVFEISW

SEQ ID NO: 62 - protein, 93 aa, Homo sapiens
MKMASTRCKLARYLEDLEDVDLKKFKMHLEDYPPQKGCIPLPRGQTEKADHVDLATLMIDFNGEEKAWAMAVWIFAAINRRDLYEKAKRDEPK

SEQ ID NO: 63 - protein, 91 aa, Mus sp.
MTSVRCKLAQYLEDLEDVDLKKFKMHLEDYPPEKGCIPVPRGQMEKADHLDLATLMIDFNGEEKAWAMAVWIFAAINRRDLWEKAKKDQPE

SEQ ID NO: 64 - protein, 195 aa, Homo sapiens
MGRARDAILDALENLTAEELKKFKLKLLSVPLREGYGRIPRGALLSMDALDLTDKLVSFYLETYGAELTANVLRDMGLQEMAGQLQAATHQGSGAAPAGIQAPPQSAAKPGLHFIDQHRAALIARVTNVEWLLDALYGKVLTDEQYQAVRAEPTNPSKMRKLFSFTPAWNWTCKDLLLQALRESQSYLVEDLERS

SEQ ID NO: 65 - protein, 193 aa, Mus sp.
MGRARDAILDALENLSGDELKKFKMKLLTVQLREGYGRIPRGALLQMDAIDLTDKLVSYYLESYGLELTMTVLRDMGLQELAEQLQTTKEESGAVAAAASVPAQSTARTGHFVDQHRQALIARVTEVDGVLDALHGSVLTEGQYQAVRAETTSQDKMRKLFSFVPSWNLTCKDSLLQALKEIHPYLVMDLEQS

SEQ ID NO: 66 - protein, 1210 aa, Homo sapiens [sequence omitted]

SEQ ID NO: 67 - protein, 497 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGM

SEQ ID NO: 68 - protein, 514 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 69 - protein, 504 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTDAVQSGPHMTPYSLLKEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 70 - protein, 488 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 71 - protein, 412 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTVTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 72 - protein, 147 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYETPSPLRITLLVQERRRKKRKSCRGCCQA

SEQ ID NO: 73 - protein, 5 aa, Artificial Sequence
  Description of Artificial Sequence Synthetic peptide
LPETG

SEQ ID NO: 74 - protein, 123 aa, Artificial Sequence
  Description of Artificial Sequence Synthetic polypeptide
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCNALSGLPPYATTYWGQGTQVTVSSKKK

SEQ ID NO: 75 - protein, 122 aa, Artificial Sequence
  Description of Artificial Sequence Synthetic polypeptide
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSS

SEQ ID NO: 76 - protein, 123 aa, Artificial Sequence
  Description of Artificial Sequence Synthetic polypeptide
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSC

SEQ ID NO: 77 - protein, 124 aa, Artificial Sequence
  Description of Artificial Sequence Synthetic polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 78 - protein, 124 aa, Artificial Sequence
  Description of Artificial Sequence Synthetic polypeptide
EVQLVESGGGSVQTGGSLRLSCAVSGNIGSSYCMGWFRQAPGKKREAVARIVRDGATGYADYVKGRFTISRDSAKNTLYLQMNRLIPEDTAIYYCAADLPPGCLTQAIWNFGYRGQGTLVTVSS

SEQ ID NO: 79 - protein, 125 aa, Artificial Sequence
  Description of Artificial Sequence Synthetic polypeptide
EVQLVESGGGSVQTGGSLRLSCAVSGNIGSSYCMGWFRQAPGKKREAVARIVRDGATGYADYVKGRFTISRDSAKNTLYLQMNRLIPEDTAIYYCAADLPPGCLTQAIWNFGYRGQGTLVTVSSC

SEQ ID NO: 80 - protein, 115 aa, Artificial Sequence
  Description of Artificial Sequence Synthetic polypeptide
EVQLLESGGGLVQPGGSLRLSCAASGFTFSTFSMNWVRQAPGKGLEWVSYISRTSKTIYYADSVKGRFTISRDNSKNTLYLQMNSLRAEDTAVYYCARGRFFDYWGQGTLVTVSS

SEQ ID NO: 81 - protein, 116 aa, Artificial Sequence
  Description of Artificial Sequence Synthetic polypeptide
EVQLLESGGGLVQPGGSLRLSCAASGFTFSTFSMNWVRQAPGKGLEWVSYISRTSKTIYYADSVKGRFTISRDNSKNTLYLQMNSLRAEDTAVYYCARGRFFDYWGQGTLVTVSSC

SEQ ID NO: 82 - protein, 112 aa, Artificial Sequence
  Description of Artificial Sequence Synthetic polypeptide
QLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSS

SEQ ID NO: 83 - protein, 113 aa, Artificial Sequence
  Description of Artificial Sequence Synthetic polypeptide
QLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSC

SEQ ID NO: 84 - protein, 117 aa, Artificial Sequence
  Description of Artificial Sequence Synthetic polypeptide
QLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSLPETG

SEQ ID NO: 85 - protein, 116 aa, Artificial Sequence
  Description of Artificial Sequence Synthetic polypeptide
EVQLVQPGGSLRLSCAVSGSISNFNAMGWYRQAPGKQRELVATITRGGSTDYAASVKGRFTISRDSAANTVYLQMNSLKPEDTAVYYCNAQRSPLILTTSRTHNYWGQGTQVTVSS

SEQ ID NO: 86 - protein, 115 aa, Artificial Sequence
  Description of Artificial Sequence Synthetic polypeptide
QVQLVQPGGSLKLSCAASGSIVSINTMAWYRQAPGKQRELIAFITSGGSTNYADSVKGRFTISRDNAKNTMYLQMSSLKPEDTAVYYCNADALYDGLQGLTRGYWGQGTRVTVST

SEQ ID NO: 87 - protein, 114 aa, Artificial Sequence
  Description of Artificial Sequence Synthetic polypeptide
VQRQESGGGVWQAGGSLRLSCTASGAVYNIDVMGWYRQAPGKERELVAGIASDESTSYGDSVKGRFLISMDRAKNTVFLKMDNLRPEDTAVYTCRANINRYDYWGTGSQVTVSS

SEQ ID NO: 88 - protein, 120 aa, Artificial Sequence
  Description of Artificial Sequence Synthetic polypeptide
QVQRQESGGGLVQPGGSLRLSCAASGDIFRVNDMAWYRQAPGKQRELVATITRGGGVAYADSVKGRFTISRDNSKNTVALQMNSLKPDDTAVYYCNADVQVGRNYEYNYWGQGTQVTVSS

SEQ ID NO: 89 - protein, 110 aa, Artificial Sequence
  Description of Artificial Sequence Synthetic polypeptide
QVQLVQPGGSLRLSCAASGRDFSGSTMGWFRQAPGKERELVAQIAWSGSIYTSRAATGRFDISRDNDKNMMWLQMNRLKPEDTGLYSCAAGKSRDDYDVWGQGTQVTVSS

SEQ ID NO: 90 - protein, 117 aa, Artificial Sequence
  Description of Artificial Sequence Synthetic polypeptide
EVQLVQPGGSLRLSCKASGNIFRMVDMGWFRQVPGKQRELVARINSDGVASYSDSVRGRFTISGSLAVNTVYLDMTNLRSEDTAVYVCNANFDHLIGWNVPARNDAWGQGTRVTVAE

SEQ ID NO: 91 - protein, 111 aa, Artificial Sequence
  Description of Artificial Sequence Synthetic polypeptide
EVQLVQPGGSLRLSCAASEKIFSITAMGWYRQAPGKQRELVASIASGGTTNYADSVKGRFTISRGNAKNTVYLQMNSLEAEDTAVYYCHAESEENWLRDYWGQGIQVTVSS

SEQ ID NO: 92 - protein, 117 aa, Artificial Sequence
  Description of Artificial Sequence Synthetic polypeptide
QVQLVESVGGWESGGFLRLSCQASGSVRNINFIGWYRQAPGKQREPVAGITASGRIGYVNSVKGRFTISRDTAKNTVYLQMDKLKPEDTGVYLCNAKWLGNEGTAYWGQGTQVTVTS

SEQ ID NO: 93 - protein, 130 aa, Artificial Sequence
  Description of Artificial Sequence Synthetic polypeptide
QVQLVETGGGLVQPGGSLRLSCVASGFKFDAYAMSWARQAPGKWLEWVSDISWNGVSEYYAESMKGRFTISRDNAKNTVYLQMSSLKPEDTAVYYCAKQARPQEVGVPTFQVTGYGMDYWGKGALVTASS

SEQ ID NO: 94 - protein, 124 aa, Artificial Sequence
  Description of Artificial Sequence Synthetic polypeptide
EVQVQESGGGLVQPGGSLRLSCAASGSFYSIVAMGWYRQAPGQQRELVATVTSGGSGGSVTNYADSVKGRFTISRDNAKNTVDLQMNSLKPDDTAVYSCWARVRVTPERFFSYWDRGTQVTVAS

SEQ ID NO: 95 - protein, 123 aa, Artificial Sequence
  Description of Artificial Sequence Synthetic polypeptide
QVQLVETGGGLVAPGGSLTLSCVASGSIFSIYSMGWYRQAPGKERELVADTTAGNTPHYGESVRGRFTISSISRDSAKNTVYLQMSDLKPDDTAVYYCAMGISHNWYAPSIYWGRGTQVTVSS

SEQ ID NO: 96 - protein, 128 aa, Artificial Sequence
  Description of Artificial Sequence Synthetic polypeptide
GGALAQPGGSLTLSCAVSGFTFDYDAAELRDYVLGWIRQVPGKEPEGVSCSSRDGTAYYSDSVKGRFTIYRDNQKNTVYLQMNSLKPDDTAVYYCAAHDLIVYYNDFPLCSTYDYDYWGQGIQVTVSS

SEQ ID NO: 97 - protein, 8 aa, Artificial Sequence
  Description of Artificial Sequence Synthetic peptide
GSISNFNA

SEQ ID NO: 98 - protein, 8 aa, Artificial Sequence
  Description of Artificial Sequence Synthetic peptide
GSIVSINT

SEQ ID NO: 99 - protein, 8 aa, Artificial Sequence
  Description of Artificial Sequence Synthetic peptide
GAVYNIDV

SEQ ID NO: 100 - protein, 8 aa, Artificial Sequence
  Description of Artificial Sequence Synthetic peptide
GDIFRVND

SEQ ID NO: 101 - protein, 8 aa, Artificial Sequence
  Description of Artificial Sequence Synthetic peptide
GRDFSGST

SEQ ID NO: 102 - protein, 8 aa, Artificial Sequence
  Description of Artificial Sequence Synthetic peptide
GNIFRMVD

SEQ ID NO: 103 - protein, 8 aa, Artificial Sequence
  Description of Artificial Sequence Synthetic peptide
EKIFSITA

SEQ ID NO: 104 - protein, 8 aa, Artificial Sequence
  Description of Artificial Sequence Synthetic peptide
GSVRNINF

SEQ ID NO: 105 - protein, 8 aa, Artificial Sequence
  Description of Artificial Sequence Synthetic peptide
GFKFDAYA

SEQ ID NO: 106 - protein, 8 aa, Artificial Sequence
  Description of Artificial Sequence Synthetic peptide
GSFYSIVA

SEQ ID NO: 107 - protein, 8 aa, Artificial Sequence
  Description of Artificial Sequence Synthetic peptide
GSIFSIYS

SEQ ID NO: 108 - protein, 8 aa, Artificial Sequence
  Description of Artificial Sequence Synthetic peptide
AAELRDYV

SEQ ID NO: 109 - protein, 7 aa, Artificial Sequence
  Description of Artificial Sequence Synthetic peptide
ITRGGST

SEQ ID NO: 110 - protein, 7 aa, Artificial Sequence
  Description of Artificial Sequence Synthetic peptide
ITSGGST

SEQ ID NO: 111 - protein, 7 aa, Artificial Sequence
  Description of Artificial Sequence Synthetic peptide
IASDEST

SEQ ID NO: 112 - protein, 7 aa, Artificial Sequence
  Description of Artificial Sequence Synthetic peptide
ITRGGGV

SEQ ID NO: 113 - protein, 7 aa, Artificial Sequence
  Description of Artificial Sequence Synthetic peptide
IAWSGSI

SEQ ID NO: 114 - protein, 7 aa, Artificial Sequence
  Description of Artificial Sequence Synthetic peptide
INSDGVA

SEQ ID NO: 115 - protein, 7 aa, Artificial Sequence
  Description of Artificial Sequence Synthetic peptide
IASGGTT

SEQ ID NO: 116 - protein, 7 aa, Artificial Sequence
  Description of Artificial Sequence Synthetic peptide
ITASGRI

SEQ ID NO: 117 - protein, 8 aa, Artificial Sequence
  Description of Artificial Sequence Synthetic peptide
ISWNGVSE

SEQ ID NO: 118 - protein, 8 aa, Artificial Sequence
  Description of Artificial Sequence Synthetic peptide
VTSGGSGG

SEQ ID NO: 119 - protein, 7 aa, Artificial Sequence
  Description of Artificial Sequence Synthetic peptide
TTAGNTP

SEQ ID NO: 120 - protein, 7 aa, Artificial Sequence
  Description of Artificial Sequence Synthetic peptide
SSRDGTA

SEQ ID NO: 121 - protein, 17 aa, Artificial Sequence
  Description of Artificial Sequence Synthetic peptide
NAQRSPLILTTSRTHNY

SEQ ID NO: 122 - protein, 16 aa, Artificial Sequence
  Description of Artificial Sequence Synthetic peptide
NADALYDGLQGLTRGY

SEQ ID NO: 123 - protein, 9 aa, Artificial Sequence
  Description of Artificial Sequence Synthetic peptide
RANINRYDY

SEQ ID NO: 124 - protein, 14 aa, Artificial Sequence
  Description of Artificial Sequence Synthetic peptide
NADVQVGRNYEYNY

SEQ ID NO: 125 - protein, 11 aa, Artificial Sequence
  Description of Artificial Sequence Synthetic peptide
AAGKSRDDYDV

SEQ ID NO: 126 - protein, 18 aa, Artificial Sequence
  Description of Artificial Sequence Synthetic peptide
NANFDHLIGWNVPARNDA

SEQ ID NO: 127 - protein, 12 aa, Artificial Sequence
  Description of Artificial Sequence Synthetic peptide
HAESEENWLRDY

SEQ ID NO: 128 - protein, 12 aa, Artificial Sequence
  Description of Artificial Sequence Synthetic peptide
NAKWLGNEGTAY

SEQ ID NO: 129 - protein, 23 aa, Artificial Sequence
  Description of Artificial Sequence Synthetic peptide
AKQARPQEVGVPTFQVTGYGMDY

SEQ ID NO: 130 - protein, 17 aa, Artificial Sequence
  Description of Artificial Sequence Synthetic peptide
YSCWARVRVTPERFFSY

SEQ ID NO: 131 - protein, 17 aa, Artificial Sequence
  Description of Artificial Sequence Synthetic peptide
YYCAMGISHNWYAPSIY

SEQ ID NO: 132 - protein, 22 aa, Artificial Sequence
  Description of Artificial Sequence Synthetic peptide
AAHDLIVYYNDFPLCSTYDYDY

SEQ ID NO: 133 - protein, 157 aa, Artificial Sequence
  Description of Artificial Sequence Synthetic polypeptide
MDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKL

SEQ ID NO: 134 - protein, 157 aa, Artificial Sequence
  Description of Artificial Sequence Synthetic polypeptide
MDLGKKLLEAARAGQDDEVRILMANGADVNASDRWGWTPLHLAAWWGHLEIVEVLLKHGADVNAADLHGQTPLHLAAMVGHLEIVEVLLKYGADVNAKDTMGATPLHLAAQSGHLEIVEVLLKNGADVNAQDKFGKTAFDISIDNGNEDLAEILQKL

SEQ ID NO: 135 - protein, 121 aa, Artificial Sequence
  Description of Artificial Sequence Synthetic polypeptide
MVSDVPRKLEVVAATPTSLLISWDAPAVTVRYYRITYGETSGGGSFAEYWALLSGGGSVQEFTVPGSKSTATISGLKPGVDYTITVYAVTSGGGSFAEYWALLSGGGSPISINYRTALESD

SEQ ID NO: 136 - protein, 106 aa, Artificial Sequence
  Description of Artificial Sequence Synthetic polypeptide
MVSDVPRKLEVVAATPTSLLISWDAPAVTVRYYRITYGETGGNSPVQEFTVPGSKSTATISGLKPGVDYTITVYAVTSGGGSFAEYWALLSGGGSPISINYRTALE

SEQ ID NO: 137 - protein, 118 aa, Artificial Sequence
  Description of Artificial Sequence Synthetic polypeptide
VSDVPRKLEVVAATPTSLLISWDAPAVTVRYYRITYGETSGGGSFAEYWALLSGGGSVQEFTVPGSKSTATISGLKPGVDYTITVYAVTSGGGSFAEYWALLSGGGSPISINYRTALE

SEQ ID NO: 138 - protein, 394 aa, Artificial Sequence
  Description of Artificial Sequence Synthetic polypeptide
CQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSTLPVYTLKERCLQVVRSLVKPENYRRLDIVRSLYEDLEDHPNVQKDLERLTQERIAHQRMGDLKNGADMNAQDKFGKTTFDISTDNGNEDLAEILQKL

SEQ ID NO: 139 - protein, 291 aa, Artificial Sequence
  Description of Artificial Sequence Synthetic polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLC

SEQ ID NO: 140 - protein, 250 aa, Artificial Sequence
  Description of Artificial Sequence Synthetic polypeptide
QVQLVESGGGLVQPGGSLTLSCTASGFTLDHYDIGWFRQAPGKEREGVSCIANSDDDTYYADSVKGRFTIFMNNAKDTVYLQMNSLKPEDTAIYYCAEARGCKRGRYEYDFWGQGTQVTVSSAAAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSC

SEQ ID NO: 141 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 142 - protein, 62 aa, Homo sapiens
TLPVYTLKERCLQVVRSLVKPENYRRLDIVRSLYEDLEDHPNVQKDLERLTQERIAHQRMGD

SEQ ID NO: 143 - protein, 62 aa, Artificial Sequence
  Description of Artificial Sequence Synthetic polypeptide
TLPVYTLKERCLQVVRSLVKPENYRRLDIVRSLFEDLEDHPNVQKDLERLTQERIAHQRMGD

SEQ ID NO: 144 - protein, 38 aa, Homo sapiens
TLKERCLQVVRSLVKPENYRRLDIVRSLYEDLEDHPNV

SEQ ID NO: 145 - protein, 38 aa, Artificial Sequence
  Description of Artificial Sequence Synthetic polypeptide
TLKERCLQVVRSLVKPENYRRLDIVRSLFEDLEDHPNV

SEQ ID NO: 146 - protein, 101 aa, Homo sapiens
RGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 147 - protein, 100 aa, Artificial Sequence
  Description of Artificial Sequence Synthetic polypeptide
RGHLWLFRDAGTHDGLLVNQTELFVPSLNVDGQPIFANITLPVYTLKERCLQVVRSLVKPENYRRLDIVRSLFDLEDHPNVQKDLERLTQERIAHQRMGD

SEQ ID NO: 148 - protein, 104 aa, Homo sapiens
HSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 149 - protein, 104 aa, Artificial Sequence
  Description of Artificial Sequence Synthetic polypeptide
HSYRGHLWLFRDAGTHDGLLVNQTELFVPSLNVDGQPIFANITLPVYTLKERCLQVVRSLVKPENYRRLDIVRSLFEDLEDHPNVQKDLERLTQERIAHQRMGD

SEQ ID NO: 150 - protein, 76 aa, Homo sapiens
PSLNVDGQPIFANITLPVYTLKERCLQVVRSLVKPENYRRLDIVRSLYEDLEDHPNVQKDLERLTQERIAHQRMGD

SEQ ID NO: 151 - protein, 76 aa, Artificial Sequence
  Description of Artificial Sequence Synthetic polypeptide
PSLNVDGQPIFANITLPVYTLKERCLQVVRSLVKPENYRRLDIVRSLFEDLEDHPNVQKDLERLTQERIAHQRMGD

SEQ ID NO: 152 - protein, 160 aa, Homo sapiens
MEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 153 - protein, 160 aa, Artificial Sequence
  Description of Artificial Sequence Synthetic polypeptide
MEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLFEDLEDHPNVQKDLERLTQERIAHQRMGD

SEQ ID NO: 154 - protein, 474 aa, Homo sapiens
MASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 155 - protein, 277 aa, Homo sapiens
MASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPG

SEQ ID NO: 156 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 157 - protein, 41 aa, Homo sapiens
VRPLQELCRQRIVATVGRENLARIPLNPVLRDYLSSFPFQI

SEQ ID NO: 158 - protein, 442 aa, Homo sapiens [sequence omitted]

SEQ ID NO: 159 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALANHYTQKSLSLSPGK

SEQ ID NO: 162 - protein, 227 aa, Artificial Sequence
  Description of Artificial Sequence Synthetic polypeptide
DKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 226 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALANHYTQKSLSLSPG

SEQ ID NO: 164 - protein, 227 aa, Artificial Sequence
  Description of Artificial Sequence Synthetic polypeptide
DKTHTCPPCPAPELLGGPCVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 227 aa, Artificial Sequence
  Description of Artificial Sequence Synthetic polypeptide
DKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALANHYTQKSLSLSPGK

SEQ ID NO: 166 - protein, 227 aa, Artificial Sequence
  Description of Artificial Sequence Synthetic polypeptide
DKTHTCPPCPAPELLGGPCVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALANHYTQKSLSLSPGK

SEQ ID NO: 167 - protein, 317 aa, Artificial Sequence
  Description of Artificial Sequence Synthetic polypeptide
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 168 - protein, 10 aa, Artificial Sequence
  Description of Artificial Sequence Synthetic peptide
DRHDSGLDSM

SEQ ID NO: 169 - protein, 5 aa, Artificial Sequence
  Description of Artificial Sequence Synthetic peptide
GGGGS

SEQ ID NO: 170 - protein, 10 aa, Artificial Sequence
  Description of Artificial Sequence Synthetic peptide
GGGGSGGGGS

SEQ ID NO: 171 - protein, 7 aa, Artificial Sequence
  Description of Artificial Sequence Synthetic peptide
GGGGAAA

SEQ ID NO: 172 - protein, 6 aa, Artificial Sequence
  Description of Artificial Sequence Synthetic peptide
GGGGAA

SEQ ID NO: 173 - protein, 5 aa, Artificial Sequence
  Description of Artificial Sequence Synthetic peptide
GGGGA

SEQ ID NO: 174 - protein, 8 aa, Artificial Sequence
  Description of Artificial Sequence Synthetic peptide
GGGGSAAA

SEQ ID NO: 175 - protein, 7 aa, Artificial Sequence
  Description of Artificial Sequence Synthetic peptide
GGGGSAA

SEQ ID NO: 176 - protein, 6 aa, Artificial Sequence
  Description of Artificial Sequence Synthetic peptide
GGGGSA

SEQ ID NO: 177 - protein, 13 aa, Artificial Sequence
  Description of Artificial Sequence Synthetic peptide
GGGGSGGGGSAAA

SEQ ID NO: 178 - protein, 12 aa, Artificial Sequence
  Description of Artificial Sequence Synthetic peptide
GGGGSGGGGSAA

SEQ ID NO: 179 - protein, 11 aa, Artificial Sequence
  Description of Artificial Sequence Synthetic peptide
GGGGSGGGGSA

SEQ ID NO: 180 - protein, 5 aa, Artificial Sequence
  Description of Artificial Sequence Synthetic peptide
SGGGG

SEQ ID NO: 181 - protein, 10 aa, Artificial Sequence
  Description of Artificial Sequence Synthetic peptide
SGGGGSGGGG

SEQ ID NO: 182 - protein, 7 aa, Artificial Sequence
  Description of Artificial Sequence Synthetic peptide
AAAGGGG

SEQ ID NO: 183 - protein, 6 aa, Artificial Sequence
  Description of Artificial Sequence Synthetic peptide
AAGGGG

SEQ ID NO: 184 - protein, 5 aa, Artificial Sequence
  Description of Artificial Sequence Synthetic peptide
AGGGG

SEQ ID NO: 185 - protein, 8 aa, Artificial Sequence
  Description of Artificial Sequence Synthetic peptide
AAASGGGG

SEQ ID NO: 186 - protein, 7 aa, Artificial Sequence
  Description of Artificial Sequence Synthetic peptide
AASGGGG

SEQ ID NO: 187 - protein, 7 aa, Artificial Sequence
  Description of Artificial Sequence Synthetic peptide
AGGGGSA

SEQ ID NO: 188 - protein, 13 aa, Artificial Sequence
  Description of Artificial Sequence Synthetic peptide
AAASGGGGSGGGG

SEQ ID NO: 189 - protein, 12 aa, Artificial Sequence
  Description of Artificial Sequence Synthetic peptide
AASGGGGSGGGG

SEQ ID NO: 190 - protein, 11 aa, Artificial Sequence
  Description of Artificial Sequence Synthetic peptide
ASGGGGSGGGG

SEQ ID NO: 191 - protein, 8 aa, Artificial Sequence
  Description of Artificial Sequence Synthetic peptide
SSGGGGSS

SEQ ID NO: 192 - protein, 7 aa, Artificial Sequence
  Description of Artificial Sequence Synthetic peptide
SGGGGSS

SEQ ID NO: 193 - protein, 6 aa, Artificial Sequence
  Description of Artificial Sequence Synthetic peptide
SGGGGS

SEQ ID NO: 194 - protein, 7 aa, Artificial Sequence
  Description of Artificial Sequence Synthetic peptide
SSGGGSS

SEQ ID NO: 195 - protein, 6 aa, Artificial Sequence
  Description of Artificial Sequence Synthetic peptide
SSGGGS

SEQ ID NO: 196 - protein, 6 aa, Artificial Sequence
  Description of Artificial Sequence Synthetic peptide
SGGGSS

SEQ ID NO: 197 - protein, 10 aa, Artificial Sequence
  Description of Artificial Sequence Synthetic peptide
SSGGGGSSAA

SEQ ID NO: 198 - protein, 9 aa, Artificial Sequence
  Description of Artificial Sequence Synthetic peptide
SSGGGGSAA

SEQ ID NO: 199 - protein, 8 aa, Artificial Sequence
  Description of Artificial Sequence Synthetic peptide
SSGGGGAA

SEQ ID NO: 200 - protein, 10 aa, Artificial Sequence
  Description of Artificial Sequence Synthetic peptide
AASSGGGGSS

SEQ ID NO: 201 - protein, 9 aa, Artificial Sequence
  Description of Artificial Sequence Synthetic peptide
AASGGGGSS

SEQ ID NO: 202 - protein, 8 aa, Artificial Sequence
  Description of Artificial Sequence Synthetic peptide
AAGGGGSS

SEQ ID NO: 203 - protein, 234 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDYKDDDDK

SEQ ID NO: 204 - protein, 39 aa, Artificial Sequence
  Description of Artificial Sequence Synthetic polypeptide
SKYIAWPLQGWQATFGGGDHPPKSDLVPRGSDYKDDDDK

SEQ ID NO: 205 - protein, 242 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGS

SEQ ID NO: 206 - protein, 14 aa, Artificial Sequence
  Description of Artificial Sequence Synthetic peptide
HHHHHHDYKDDDDK

SEQ ID NO: 207 - protein, 9 aa, Artificial Sequence
  Description of Artificial Sequence Synthetic peptide
YPYDVPDYA

SEQ ID NO: 208 - protein, 14 aa, Artificial Sequence
  Description of Artificial Sequence Synthetic peptide
MHHHHHHENLYFQG

SEQ ID NO: 209 - protein, 19 aa, Unknown
  Description of Unknown mammalian signal peptide
MGWSCIILFLVATATGVHS

SEQ ID NO: 210 - protein, 438 aa, Artificial Sequence
  Description of Artificial Sequence Synthetic polypeptide
CDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPG

SEQ ID NO: 211 - protein, 439 aa, Artificial Sequence
  Description of Artificial Sequence Synthetic polypeptide
MASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGGGGGSDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLC

SEQ ID NO: 212 - protein, 170 aa, Artificial Sequence
  Description of Artificial Sequence Synthetic polypeptide
CQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGSLPVYTLKERCLQVVRSLVKPENYRRLDIVRSLYEDLEDHPNV

SEQ ID NO: 213 - protein, 170 aa, Artificial Sequence
  Description of Artificial Sequence Synthetic polypeptide
LPVYTLKERCLQVVRSLVKPENYRRLDIVRSLYEDLEDHPNVGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSC

SEQ ID NO: 214 - protein, 172 aa, Artificial Sequence
  Description of Artificial Sequence Synthetic polypeptide
CQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSLPVYTLKERCLQVVRSLVKPENYRRLDIVRSLYEDLEDHPNV

SEQ ID NO: 215 - protein, 172 aa, Artificial Sequence
  Description of Artificial Sequence Synthetic polypeptide
LPVYTLKERCLQVVRSLVKPENYRRLDIVRSLYEDLEDHPNVGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSC

SEQ ID NO: 216 - protein, 160 aa, Artificial Sequence
  Description of Artificial Sequence Synthetic polypeptide
CQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSLPVYTLKERCLQVVRSLVKPENYRRLDIVRSLYEDLEDHPNV

SEQ ID NO: 217 - protein, 160 aa, Artificial Sequence
  Description of Artificial Sequence Synthetic polypeptide
LPVYTLKERCLQVVRSLVKPENYRRLDIVRSLYEDLEDHPNVGGGGSQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSC

SEQ ID NO: 218 - protein, 222 aa, Artificial Sequence
  Description of Artificial Sequence Synthetic polypeptide
CQVQLQESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSHSYRGHLWLFRDAGTHDGLLVAQTELFVPSLNVDGQPIFAAITLPVYTLKERCLQVVRSLVKPENYRRLDIVRSLYEDLEDHPNVQKDLERLTQERIAHQRMGD

SEQ ID NO: 219 - protein, 194 aa, Artificial Sequence
  Description of Artificial Sequence Synthetic polypeptide
CQVQLQESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSPSLNVDGQPIFAAITLPVYTLKERCLQVVRSLVKPENYRRLDIVRSLYEDLEDHPNVQKDLERLTQERIAHQRMGD

SEQ ID NO: 220 - protein, 180 aa, Artificial Sequence
  Description of Artificial Sequence Synthetic polypeptide
CQVQLQESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSTLPVYTLKERCLQVVRSLVKPENYRRLDIVRSLYEDLEDHPNVQKDLERLTQERIAHQRMGD

SEQ ID NO: 221 - protein, 180 aa, Artificial Sequence
  Description of Artificial Sequence Synthetic polypeptide
TLPVYTLKERCLQVVRSLVKPENYRRLDIVRSLYEDLEDHPNVQKDLERLTQERIAHQRMGDGGGGSQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSC

SEQ ID NO: 222 - protein, 474 aa, Artificial Sequence
  Description of Artificial Sequence Synthetic polypeptide
DYKDDDDKGSSGHHHHHHENLYFQGMPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDGGGGGEFAMAQVQLQESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSS

SEQ ID NO: 223 - protein, 421 aa, Artificial Sequence
  Description of Artificial Sequence Synthetic polypeptide
DYKDDDDKGSSGHHHHHHENLYFQGMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDGGGGGEFAMAQVQLQESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSS

SEQ ID NO: 224 - protein, 394 aa, Artificial Sequence
  Description of Artificial Sequence Synthetic polypeptide
QLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGC

SEQ ID NO: 225 - protein, 395 aa, Artificial Sequence
  Description of Artificial Sequence Synthetic polypeptide
MASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGGGGGSQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSC

SEQ ID NO: 226 - protein, 461 aa, Artificial Sequence
  Description of Artificial Sequence Synthetic polypeptide
TLPVYTLKERCLQVVRSLVKPENYRRLDIVRSLYEDLEDHPNVQKDLERLTQERIAHQRMGDGGGGSQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGC

SEQ ID NO: 227 - protein, 226 aa, Artificial Sequence
  Description of Artificial Sequence Synthetic polypeptide
CTLPVYTLKERCLQVVRSLVKPENYRRLDIVRSLYEDLEDHPNVQKDLERLTQERIAHQRMGDGGGGSQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSVRPLQELCRQRIVATVGRENLARIPLNPVLRDYLSSFPFQI

SEQ ID NO: 228 - protein, 226 aa, Artificial Sequence
  Description of Artificial Sequence Synthetic polypeptide
VRPLQELCRQRIVATVGRENLARIPLNPVLRDYLSSFPFQIGGGGSTLPVYTLKERCLQVVRSLVKPENYRRLDIVRSLYEDLEDHPNVQKDLERLTQERIAHQRMGDGGGGSQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSC

SEQ ID NO: 229 - protein, 246 aa, Artificial Sequence
  Description of Artificial Sequence Synthetic polypeptide
CTLPVYTLKERCLQVVRSLVKPENYRRLDIVRSLYEDLEDHPNVQKDLERLTQERIAHQRMGDGGGGSGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGSGGGGSVRPLQELCRQRIVATVGRENLARIPLNPVLRDYLSSFPFQI

SEQ ID NO: 230 - protein, 195 aa, Artificial Sequence
  Description of Artificial Sequence Synthetic polypeptide
TLPVYTLKERCLQVVRSLVKPENYRRLDIVRSLFEDLEDHPNVQKDLERLTQERIAHQRMGDGGGGSGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSC

SEQ ID NO: 231 - protein, 169 aa, Artificial Sequence
  Description of Artificial Sequence Synthetic polypeptide
CQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGSVRPLQELCRQRIVATVGRENLARIPLNPVLRDYLSSFPFQI

SEQ ID NO: 232 - protein, 169 aa, Artificial Sequence
  Description of Artificial Sequence Synthetic polypeptide
VRPLQELCRQRIVATVGRENLARIPLNPVLRDYLSSFPFQIGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSC

SEQ ID NO: 233 - protein, 409 aa, Artificial Sequence
  Description of Artificial Sequence Synthetic polypeptide
CQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGSGGGGSASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPG

SEQ ID NO: 234 - protein, 421 aa, Artificial Sequence
  Description of Artificial Sequence Synthetic polypeptide
HHHHHHGDDDDKASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGGGGGSGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSC

SEQ ID NO: 235 - protein, 481 aa, Artificial Sequence
  Description of Artificial Sequence Synthetic polypeptide
TLPVYTLKERCLQVVRSLVKPENYRRLDIVRSLYEDLEDHPNVQKDLERLTQERIAHQRMGDGGGGSGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGSGGGGSASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGC

SEQ ID NO: 236 - protein, 171 aa, Artificial Sequence
  Description of Artificial Sequence Synthetic polypeptide
CQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSVRPLQELCRQRIVATVGRENLARIPLNPVLRDYLSSFPFQI

SEQ ID NO: 237 - protein, 171 aa, Artificial Sequence
  Description of Artificial Sequence Synthetic polypeptide
VRPLQELCRQRIVATVGRENLARIPLNPVLRDYLSSFPFQIGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSC

SEQ ID NO: 238 - protein, 159 aa, Artificial Sequence
  Description of Artificial Sequence Synthetic polypeptide
CQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSVRPLQELCRQRIVATVGRENLARIPLNPVLRDYLSSFPFQI

SEQ ID NO: 239 - protein, 159 aa, Artificial Sequence
  Description of Artificial Sequence Synthetic polypeptide
VRPLQELCRQRIVATVGRENLARIPLNPVLRDYLSSFPFQIGGGGSQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSC

SEQ ID NO: 240 - protein, 338 aa, Artificial Sequence
  Description of Artificial Sequence Synthetic polypeptide
CQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSLPVYTLKERCLQVVRSLVKPENYRRLDIVRSLYEDLEDHPNV

SEQ ID NO: 241 - protein, 358 aa, Artificial Sequence
  Description of Artificial Sequence Synthetic polypeptide
CQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSTLPVYTLKERCLQVVRSLVKPENYRRLDIVRSLYEDLEDHPNVQKDLERLTQERIAHQRMGD

SEQ ID NO: 242 - protein, 572 aa, Artificial Sequence
  Description of Artificial Sequence Synthetic polypeptide
CQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPG

SEQ ID NO: 243 - protein, 358 aa, Artificial Sequence
  Description of Artificial Sequence Synthetic polypeptide
CDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSTLPVYTLKERCLQVVRSLVKPENYRRLDIVRSLYEDLEDHPNVQKDLERLTQERIAHQRMGD

SEQ ID NO: 244 - protein, 338 aa, Artificial Sequence
  Description of Artificial Sequence Synthetic polypeptide
CDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSLPVYTLKERCLQVVRSLVKPENYRRLDIVRSLYEDLEDHPNV

SEQ ID NO: 245 - protein, 337 aa, Artificial Sequence
  Description of Artificial Sequence Synthetic polypeptide
CDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSVRPLQELCRQRIVATVGRENLARIPLNPVLRDYLSSFPFQI

SEQ ID NO: 246 - protein, 344 aa, Artificial Sequence
  Description of Artificial Sequence Synthetic polypeptide
QLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 247 - protein, 344 aa, Artificial Sequence
  Description of Artificial Sequence Synthetic polypeptide
QLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 344 aa, Artificial Sequence
  Description of Artificial Sequence Synthetic polypeptide
QLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALANHYTQKSLSLSPGC

SEQ ID NO: 249 - protein, 344 aa, Artificial Sequence
  Description of Artificial Sequence Synthetic polypeptide
QLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSDKTHTCPPCPAPELLGGPCVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 234 aa, Artificial Sequence
  Description of Artificial Sequence Synthetic polypeptide
QLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 251 - protein, 300 aa, Artificial Sequence
  Description of Artificial Sequence Synthetic polypeptide
CQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSTLPVYTLKERCLQVVRSLVKPENYRRLDIVRSLYEDLEDHPNVQKDLERLTQERIAHQRMGD

SEQ ID NO: 252 - protein, 301 aa, Artificial Sequence
  Description of Artificial Sequence Synthetic polypeptide
TLPVYTLKERCLQVVRSLVKPENYRRLDIVRSLYEDLEDHPNVQKDLERLTQERIAHQRMGDGGGGSQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 253 - protein, 354 aa, Artificial Sequence
  Description of Artificial Sequence Synthetic polypeptide
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 254 - protein, 353 aa, Artificial Sequence
  Description of Artificial Sequence Synthetic polypeptide
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGSDKTHTCPPCPAPELLGGPCVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 255 - protein, 354 aa, Artificial Sequence
  Description of Artificial Sequence Synthetic polypeptide
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGSDKTHTCPPCPAPELLGGPCVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 256 - protein, 354 aa, Artificial Sequence
  Description of Artificial Sequence Synthetic polypeptide
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALANHYTQKSLSLSPGC

SEQ ID NO: 257 - protein, 244 aa, Artificial Sequence
  Description of Artificial Sequence Synthetic polypeptide
QVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 258 - protein, 316 aa, Artificial Sequence
  Description of Artificial Sequence Synthetic polypeptide
CQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSTLPVYTLKERCLQVVRSLVKPENYRRLDIVRSLYEDLEDHPNVQKDLERLTQERIAHQRMGD

SEQ ID NO: 259 - protein, 316 aa, Artificial Sequence
  Description of Artificial Sequence Synthetic polypeptide
TLPVYTLKERCLQVVRSLVKPENYRRLDIVRSLYEDLEDHPNVQKDLERLTQERIAHQRMGDGGGGSGGGGSQVQLVESGGGLVQPGGSLTLSCTASGFTLDHYDIGWFRQAPGKEREGVSCINNSDDDTYYADSVKGRFTIFMNNAKDTVYLQMNSLKPEDTAIYYCAEARGCKRGRYEYDFWGQGTQVTVSSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 260 - protein, 356 aa, Artificial Sequence
  Description of Artificial Sequence Synthetic polypeptide
EVQLVESGGGSVQTGGSLRLSCAVSGNIGSSYCMGWFRQAPGKKREAVARIVRDGATGYADYVKGRFTISRDSAKNTLYLQMNRLIPEDTAIYYCAADLPPGCLTQAIWNFGYRGQGTLVTVSSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 261 - protein, 356 aa, Artificial Sequence
  Description of Artificial Sequence Synthetic polypeptide
EVQLVESGGGSVQTGGSLRLSCAVSGNIGSSYCMGWFRQAPGKKREAVARIVRDGATGYADYVKGRFTISRDSAKNTLYLQMNRLIPEDTAIYYCAADLPPGCLTQAIWNFGYRGQGTLVTVSSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALANHYTQKSLSLSPGC

SEQ ID NO: 262 - protein, 388 aa, Artificial Sequence
  Description of Artificial Sequence Synthetic polypeptide
DLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 263 - protein, 388 aa, Artificial Sequence
  Description of Artificial Sequence Synthetic polypeptide
DLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSDKTHTCPPCPAPELLGGPCVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 392 aa, Artificial Sequence
  Description of Artificial Sequence Synthetic polypeptide
DKTHTCPPCPAPELLGGPCVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKL

SEQ ID NO: 265 - protein, 388 aa, Artificial Sequence
  Description of Artificial Sequence Synthetic polypeptide
DLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALANHYTQKSLSLSPGC

SEQ ID NO: 266 - protein, 398 aa, Artificial Sequence
  Description of Artificial Sequence Synthetic polypeptide
DLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSGGGGSEPKSSDKTHTCPPCPAPELLGGPCVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 350 aa, Artificial Sequence
  Description of Artificial Sequence Synthetic polypeptide
CDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSTLPVYTLKERCLQVVRSLVKPENYRRLDIVRSLYEDLEDHPNVQKDLERLTQERIAHQRMGD

SEQ ID NO: 268 - protein, 350 aa, Artificial Sequence
  Description of Artificial Sequence Synthetic polypeptide
TLPVYTLKERCLQVVRSLVKPENYRRLDIVRSLYEDLEDHPNVQKDLERLTQERIAHQRMGDGGGGSDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 269 - protein, 246 aa, Artificial Sequence
  Description of Artificial Sequence Synthetic polypeptide
EVQLVESGGGSVQTGGSLRLSCAVSGNIGSSYCMGWFRQAPGKKREAVARIVRDGATGYADYVKGRFTISRDSAKNTLYLQMNRLIPEDTAIYYCAADLPPGCLTQAIWNFGYRGQGTLVTVSSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 270 - protein, 237 aa, Artificial Sequence
  Description of Artificial Sequence Synthetic polypeptide
EVQLLESGGGLVQPGGSLRLSCAASGFTFSTFSMNWVRQAPGKGLEWVSYISRTSKTIYYADSVKGRFTISRDNSKNTLYLQMNSLRAEDTAVYYCARGRFFDYWGQGTLVTVSSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 271 - protein, 283 aa, Artificial Sequence
  Description of Artificial Sequence Synthetic polypeptide
DLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 272 - protein, 246 aa, Artificial Sequence
  Description of Artificial Sequence Synthetic polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 273 - protein, 239 aa, Artificial Sequence
  Description of Artificial Sequence Synthetic polypeptide
QLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGGGSEPKSSDKTHTCPPCPGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - protein, 422 aa, Artificial Sequence
  Description of Artificial Sequence Synthetic polypeptide
MDYKDDDDKGSSGHHHHHHENLYFQGMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDGGGGGEFAMAQVQLQESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSGGQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSS

SEQ ID NO: 275 - protein, 390 aa, Artificial Sequence
  Description of Artificial Sequence Synthetic polypeptide
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGDGGGGSAAADLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSDYKDDDDK

SEQ ID NO: 276 - protein, 216 aa, Artificial Sequence
  Description of Artificial Sequence Synthetic polypeptide
MTLKERCLQVVRSLVKPENYRRLDIVRSLYEDLEDHPNVGGGGSAAADLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLGGGGSDYKDDDDK

SEQ ID NO: 277 - protein, 5 aa, Artificial Sequence
  Description of Artificial Sequence Synthetic peptide
  MOD_RES (2)..(2): Beta-alanine
  MOD_RES (4)..(4): Beta-alanine
RARAR

SEQ ID NO: 278 - protein, 5 aa, Artificial Sequence
  Description of Artificial Sequence Synthetic peptide
  MOD_RES (2)..(2): Beta-alanine
  MOD_RES (4)..(4): Beta-alanine
RAHAR

SEQ ID NO: 279 - protein, 5 aa, Artificial Sequence
  Description of Artificial Sequence Synthetic peptide
  MOD_RES (2)..(2): Beta-alanine
  MOD_RES (4)..(4): Beta-alanine
HARAH

SEQ ID NO: 280 - protein, 19 aa, Artificial Sequence
  Description of Artificial Sequence Synthetic peptide
NLSKRPAAIKKAGQAKKKK

SEQ ID NO: 281 - protein, 9 aa, Artificial Sequence
  Description of Artificial Sequence Synthetic peptide
PAAKRVKLD

SEQ ID NO: 282 - protein, 11 aa, Artificial Sequence
  Description of Artificial Sequence Synthetic peptide
RQRRNELKRSF

SEQ ID NO: 283 - protein, 27 aa, Artificial Sequence
  Description of Artificial Sequence Synthetic peptide
RMRKFKNKGKDTAELRRRRVEVSVELR

SEQ ID NO: 284 - protein, 14 aa, Artificial Sequence
  Description of Artificial Sequence Synthetic peptide
KAKKDEQILKRRNV

SEQ ID NO: 285 - protein, 8 aa, Artificial Sequence
  Description of Artificial Sequence Synthetic peptide
VSRKRPRP

SEQ ID NO: 286 - protein, 8 aa, Artificial Sequence
  Description of Artificial Sequence Synthetic peptide
PPKKARED

SEQ ID NO: 287 - protein, 8 aa, Artificial Sequence
  Description of Artificial Sequence Synthetic peptide
PQPKKKPL

SEQ ID NO: 288 - protein, 12 aa, Artificial Sequence
  Description of Artificial Sequence Synthetic peptide
SALIKKKKKMAP

SEQ ID NO: 289 - protein, 5 aa, Artificial Sequence
  Description of Artificial Sequence Synthetic peptide
DRLRR

SEQ ID NO: 290 - protein, 7 aa, Artificial Sequence
  Description of Artificial Sequence Synthetic peptide
PKQKKRK

SEQ ID NO: 291 - protein, 10 aa, Artificial Sequence
  Description of Artificial Sequence Synthetic peptide
RKLKKKIKKL

SEQ ID NO: 292 - protein, 10 aa, Artificial Sequence
  Description of Artificial Sequence Synthetic peptide
REKKKFLKRR

SEQ ID NO: 293 - protein, 20 aa, Artificial Sequence
  Description of Artificial Sequence Synthetic peptide
KRKGDEVDGVDEVAKKKSKK

SEQ ID NO: 294 - protein, 17 aa, Artificial Sequence
  Description of Artificial Sequence Synthetic peptide
RKCLQAGMNLEARKTKK

SEQ ID NO: 295 - protein, 7 aa, Artificial Sequence
  Description of Artificial Sequence Synthetic peptide
PKKKRKV

SEQ ID NO: 296 - protein, 7 aa, Artificial Sequence
  Description of Artificial Sequence Synthetic peptide
FGFGRGR

SEQ ID NO: 297 - protein, 4 aa, Artificial Sequence
  Description of Artificial Sequence Synthetic peptide
  MOD_RES (4)..(4): L-naphthylalanine
FGFA

SEQ ID NO: 298 - protein, 8 aa, Artificial Sequence
  Description of Artificial Sequence Synthetic peptide
FGFGRGRQ

SEQ ID NO: 299 - protein, 8 aa, Artificial Sequence
  Description of Artificial Sequence Synthetic peptide
FGFGRRRQ

SEQ ID NO: 300 - protein, 8 aa, Artificial Sequence
  Description of Artificial Sequence Synthetic peptide
FGFRRRRQ

SEQ ID NO: 301 - protein, 7 aa, Artificial Sequence
  Description of Artificial Sequence Synthetic peptide
  MOD_RES (2)..(2): L-naphthylalanine
FARRRRQ

SEQ ID NO: 302 - protein, 6 aa, Artificial Sequence
  Description of Artificial Sequence Synthetic peptide
  MOD_RES (2)..(2): L-naphthylalanine
FARRRQ

SEQ ID NO: 303 - protein, 6 aa, Artificial Sequence
  Description of Artificial Sequence Synthetic peptide
  MOD_RES (2)..(2): L-naphthylalanine
FARRRC

SEQ ID NO: 304 - protein, 6 aa, Artificial Sequence
  Description of Artificial Sequence Synthetic peptide
  MOD_RES (2)..(2): L-naphthylalanine
  MOD_RES (6)..(6): L-selenocysteine
FARRRC

SEQ ID NO: 305 - protein, 6 aa, Artificial Sequence
  Description of Artificial Sequence Synthetic peptide
  MOD_RES (4)..(4): L-naphthylalanine
RRRAFQ

SEQ ID NO: 306 - protein, 6 aa, Artificial Sequence
  Description of Artificial Sequence Synthetic peptide
  MOD_RES (5)..(5): L-naphthylalanine
RRRRAF

SEQ ID NO: 307 - protein, 6 aa, Artificial Sequence
  Description of Artificial Sequence Synthetic peptide
  MOD_RES (2)..(2): L-naphthylalanine
FARRRR

SEQ ID NO: 308 - protein, 7 aa, Artificial Sequence
  Description of Artificial Sequence Synthetic peptide
  MOD_RES (5)..(5): L-naphthylalanine
RRFRARQ

SEQ ID NO: 309 - protein, 7 aa, Artificial Sequence
  Description of Artificial Sequence Synthetic peptide
  MOD_RES (6)..(6): L-naphthylalanine
FRRRRAQ

SEQ ID NO: 310 - protein, 7 aa, Artificial Sequence
  Description of Artificial Sequence Synthetic peptide
  MOD_RES (3)..(3): L-naphthylalanine
RRAFRRQ

SEQ ID NO: 311 - protein, 8 aa, Artificial Sequence
  Description of Artificial Sequence Synthetic peptide
CRRRRFWQ

SEQ ID NO: 312 - protein, 8 aa, Artificial Sequence
  Description of Artificial Sequence Synthetic peptide
  MOD_RES (3)..(3): L-naphthylalanine
FFARRRRQ

SEQ ID NO: 313 - protein, 8 aa, Artificial Sequence
  Description of Artificial Sequence Synthetic peptide
  MOD_RES (6)..(6): L-naphthylalanine
RFRFRARQ

SEQ ID NO: 314 - protein, 8 aa, Artificial Sequence
  Description of Artificial Sequence Synthetic peptide
  MOD_RES (1)..(1): L-selenocysteine
CRRRRFWQ

SEQ ID NO: 315 - protein, 8 aa, Artificial Sequence
  Description of Artificial Sequence Synthetic peptide
  MOD_RES (2)..(2): L-naphthylalanine
FARRRRQK

SEQ ID NO: 316 - protein, 8 aa, Artificial Sequence
  Description of Artificial Sequence Synthetic peptide
  MOD_RES (2)..(2): L-naphthylalanine
FARRRRQC

SEQ ID NO: 317 - protein, 8 aa, Artificial Sequence
  Description of Artificial Sequence Synthetic peptide
  MOD_RES (2)..(2): L-naphthylalanine
FARRRRRQ

SEQ ID NO: 318 - protein, 9 aa, Artificial Sequence
  Description of Artificial Sequence Synthetic peptide
  MOD_RES (5)..(5): L-naphthylalanine
  MOD_RES (8)..(8): L-norleucine
RRRRAFDLC

SEQ ID NO: 319 - protein, 5 aa, Artificial Sequence
  Description of Artificial Sequence Synthetic peptide
  MOD_RES (2)..(2): L-naphthylalanine
FARRR

SEQ ID NO: 320 - protein, 5 aa, Artificial Sequence
  Description of Artificial Sequence Synthetic peptide
FWRRR

SEQ ID NO: 321 - protein, 5 aa, Artificial Sequence
  Description of Artificial Sequence Synthetic peptide
  MOD_RES (4)..(4): L-naphthylalanine
RRRAF

SEQ ID NO: 322 - protein, 5 aa, Artificial Sequence
  Description of Artificial Sequence Synthetic peptide
RRRWF

SEQ ID NO: 323 - DNA, 1032 bp, Artificial Sequence
  Description of Artificial Sequence Synthetic polynucleotide
cagttgcagctagttgaaagtggtggcggtttagttcagccaggcggttcattaaagttatcatgtgctgcttcaggcttcaccttttcaagatacgccatgagttggtaccgtcaagcccctggaaaggagagagagtctgttgctaggatttcatctggcggaggtacgatctactacgctgattcagtcaaaggtaggttcaccataagtagggaggacgcaaagaacaccgtttatctgcagatgaactcccttaaacctgaggacacggccgtctactactgttatgtaggcggcttttggggccaaggtactcaggtcactgttagttccggaggaggaggatctgataaaacccatacttgtcctccctgtcccgcacctgagcttttaggaggtccttccgtatttttatttccccctaagcctaaagatactctgatgattagtcgtactcctgaggttacgtgtgttgtagtcgatgtatcccacgaggacccagaagttaagtttaattggtatgtagacggcgtggaggtccacaacgccaaaactaagcccagggaagagcagtatgccagtacatatagggtagtctcagtactgaccgtgctacaccaggattggttgaatggaaaagaatataaatgtaaagtgtctaataaggcactgcccgctccaatcgagaagacgatttccaaagccaagggccagcccagagagccccaggtttatactttgccaccaagtcgtgaagaaatgacaaagaatcaggtatcactgacgtgtctggttaaaggtttctatcctagtgacatagccgtcgagtgggaatctaatggtcaaccagagaataattataagactactccaccagtattagacagtgacggttctttctttctttacagtaaactgacagtagacaaaagtcgttggcaacagggaaatgtcttttcatgctcagtgatgcatgaggcccttcacaaccattacacccaaaaatcactatccttgagtcccggctgc

SEQ ID NO: 324 - DNA, 1032 bp, Artificial Sequence
  Description of Artificial Sequence Synthetic polynucleotide
caattgcagttggtggaatcaggcggaggtttggttcaacctggtggttctctaaaattatcttgtgctgcaagtggattcacatttagtcgttacgctatgtcatggtatagacaggcaccaggaaaagagagggagagtgtcgcaagaatcagttctggaggcggtacgatttactacgctgactcagtgaagggcaggtttactatttctagggaagacgctaaaaacaccgtatacctacaaatgaactccctgaagcccgaggatactgcagtttactattgttacgttggaggcttttggggacagggcacccaggttactgtttcttcaggtggcggtggttccgataagacacacacgtgccccccatgtcccgcaccagaacttctaggcggtccctccgtattcttattcccacccaaacctaaggatacattgatgatctcaaggacgcccgaggtcacatgcgtggtggtggacgttagtcacgaagacccagaagtaaagttcaattggtacgtagacggtgtcgaggtacacaatgccaaaacaaagccaagagaggagcaatacgcatctacctatagagtagttagtgtcttgaccgtgttgcatcaagattggcttaacggtaaagagtacaagtgcaaggttagtaataaggccttgcccgctccaattgaaaagacaatctccaaagccaaaggtcagcctcgtgagccccaagtttacaccttaccaccatctcgtgaggagatgactaagaatcaagtaagtttgacttgtctagtgaaaggcttttaccctagtgacatagccgtcgagtgggaatcaaacggccagccagagaataattataagacaacgccccccgttcttgactccgatggttccttttttttatattcaaagctaacggttgacaagtccagatggcaacaaggtaacgtctttagttgtagtgtgatgcacgaagcccttgcaaatcattatacgcaaaaatcattatccctgagtcccggatgt

SEQ ID NO: 325 - DNA, 1032 bp, Artificial Sequence
  Description of Artificial Sequence Synthetic polynucleotide
caacttcagctggtggaatccggaggaggtcttgtccaaccaggaggatcattgaagttgtcatgcgctgcatccggctttacctttagtcgttatgcaatgtcctggtatagacaagcacctggtaaggagagggagtcagttgccaggatcagtagtggaggtggaactatttactatgctgattctgtcaagggccgtttcacaatttcccgtgaggatgccaagaatacagtatatttgcaaatgaactccctgaaaccagaggatactgcagtatactattgttacgtgggtggtttctggggacagggaacgcaagtgacggtatcatcaggtggaggcggttccgacaaaacacacacctgtcctccctgccccgcccctgaattattgggaggaccatgcgtatttctattccctccaaagcccaaggacacattaatgatctctcgtacgcctgaagtgacttgcgtggtagttgacgtatcacacgaggatcctgaagtaaagtttaattggtacgtggacggtgtcgaagttcataatgctaagacgaagcccagagaagagcaatacgcttccacgtacagggtcgtatccgtactaacagttctacaccaagattggctgaatggaaaagagtataaatgcaaggtgtccaacaaggcattgcctgcccccatagaaaagacgatatcaaaagctaagggtcaaccaagagagcctcaagtatacacactaccacccagtagggaagagatgactaaaaaccaggtgtccctaacctgcctggtgaaaggcttctatccaagtgacatcgccgtcgagtgggaatctaatggacaaccagagaataattacaagacaacacctcctgtcctggattccgatggctcttttttcttgtattccaagttgactgttgacaagtcaaggtggcagcagggaaacgtcttttcttgcagtgttatgcacgaagctcttcataaccattacactcagaaatccctgagtctttcccctggtaag

SEQ ID NO: 326 - DNA, 1062 bp, Artificial Sequence
  Description of Artificial Sequence Synthetic polynucleotide
caggtgcaacttgttgagtcaggtggtggtctagtacaacctggcggtagtcttacgttatcctgtactgcctcaggatttacgctagatcactacgacataggctggtttaggcaagcacctggaaaggaaagagaaggcgtgtcttgcattaacaatagtgacgatgacacttattatgccgactccgtcaaaggaagattcacaatctttatgaacaatgcaaaagacactgtttacctgcagatgaacagtttaaagcccgaggatacggctatatattactgcgccgaagccaggggctgtaaaaggggaagatacgaatacgatttctggggccaaggcacgcaagttaccgtctcttccggaggaggaggttctgacaaaactcatacatgccctccttgtcccgcacctgagttgcttggcggcccttccgttttcctgtttccaccaaaaccaaaagacacacttatgattagtcgtacacccgaggttacgtgcgtagtggttgatgtgtcacatgaggaccccgaggttaagttcaactggtatgttgatggagtggaggtgcataatgccaaaacgaaacccagggaggagcagtatgcttccacgtatagggtagttagtgttctgactgttttacatcaagattggttgaacggcaaggagtataaatgtaaagtctcaaataaagctcttcctgctcccatcgagaaaactatctccaaggccaaaggacaacctcgtgaaccccaagtgtacacacttccacctagtcgtgaggaaatgactaagaaccaggtgtctttaacgtgtctagtaaaaggattctacccatccgatattgcagttgaatgggagtccaatggacagccagagaacaactacaaaactaccccacccgtccttgattcagacggatcattttttctttactccaaattaacagtcgataaaagtaggtggcagcagggaaatgttttctcatgtagtgtaatgcacgaagcactacacaatcactatactcaaaaaagtctgtcattaagtcctggctgt

SEQ ID NO: 327 - DNA, 1062 bp, Artificial Sequence
  Description of Artificial Sequence Synthetic polynucleotide
caagttcagctagtggagtccggcggcggccttgttcagccaggaggttcactaacgctgtcctgcacagcatccggcttcacgttagaccattacgatattggatggttcagacaggctccaggaaaagagagagagggcgtttcttgtatcaataactcagacgacgacacttattatgcagattccgttaaaggtaggtttacaatatttatgaataatgccaaagacacggtgtatttgcaaatgaactctttaaagcccgaggacacggccatctactactgcgctgaggcaagaggctgtaagagaggaagatatgaatatgacttctggggacaaggcacccaggttacggtctcaagtggcggcggtggcagtgacaagacgcacacttgtcctccctgccccgctcctgaattattgggcggtccatctgttttccttttccccccaaagcctaaagatacactgatgatatcccgtacgcccgaggtgacatgcgtcgttgtcgacgtgtcacatgaggaccctgaggttaaatttaactggtacgtagatggagtagaggtacataacgcaaaaacgaagccaagagaggaacaatacgcctccacttaccgtgtcgtatccgttttgacggtcttgcatcaagactggttaaacggcaaagaatacaagtgcaaagtttccaataaggcactgccagctcctattgaaaagacgatttctaaggcaaagggtcaacctcgtgagcctcaagtctacaccctgccaccatcacgtgaagaaatgacaaaaaaccaagtttctctgacttgtctggtgaagggcttttatccctctgacattgccgtggaatgggagtccaacggacagccagagaataactataaaacaacgccacccgttctggactcagacggatcattttttttatattccaaattgactgtggacaagagtcgttggcaacagggtaacgtattttcttgttccgtgatgcacgaagctctggccaaccattacacgcaaaaatctttgtcactatcacctggttgc

SEQ ID NO: 328 - DNA, 1068 bp, Artificial Sequence
  Description of Artificial Sequence Synthetic polynucleotide
gaggtgcagttggtcgagtccggtggtggttctgtacaaaccggtggttcacttcgtctgtcatgcgccgtttcaggtaacataggttcctcatattgcatgggatggtttaggcaagcccctggaaaaaagagagaagcagtagccagaatagtacgtgatggtgctacgggctacgctgattacgttaaaggccgtttcacaatttcaagggactccgctaagaacactctttatctgcaaatgaataggcttatcccagaggatactgccatctactattgtgctgccgatcttcccccaggctgcctaacccaggccatctggaactttggatacaggggccagggcaccttggttaccgtctcaagtggcggtggtggaagtgataaaactcacacttgccctccttgcccagccccagagcttctaggtggtccttcagtttttttatttccccctaagcccaaggacacgttgatgatttcaagaaccccagaggttacttgtgtcgtagtagacgttagtcacgaggaccccgaggtgaagttcaattggtacgtcgacggcgttgaggtacataatgctaaaaccaaaccacgtgaagagcagtatgcctctacctaccgtgtagtgtctgttttaactgtattgcatcaggactggttaaacggtaaagagtataaatgcaaggtctctaataaggccttgccagcaccaattgagaagaccatatccaaggcaaaaggccaacccagagagccacaagtttacacattgcctccttcacgtgaagaaatgaccaaaaatcaggtgtctttgacatgtttggtaaagggcttttaccccagtgacattgctgtggagtgggagagtaatggacagcccgaaaataactacaaaacgactccacccgtgcttgatagtgacggttctttcttcctatactccaaactgactgtcgacaaatcaagatggcaacagggaaatgttttcagttgcagtgtcatgcacgaagcactgcataatcactacacccaaaaatcattgtccttaagtcccggttgc

SEQ ID NO: 329 - DNA, 1068 bp, Artificial Sequence
  Description of Artificial Sequence Synthetic polynucleotide
gaggtacaacttgtggagtcaggcggtggctccgtgcaaacaggcggtagtctaagactttcatgtgcagtatccggcaacattggctctagttattgcatgggatggttcaggcaagcacccggaaagaagcgtgaggccgtagcacgtattgtccgtgacggagctactggttacgcagactacgtaaagggtcgttttaccataagtcgtgattcagctaaaaacactctttacctacaaatgaaccgtctaatcccagaagacaccgcaatctattattgcgccgcagatttgccccctggatgcttgactcaagcaatctggaattttggatatagaggacagggtaccttggtaaccgtttcatcaggcggcggtggttccgataagacgcatacctgtcccccatgtcctgcacctgagttactaggtggaccttcagtttttttatttccaccaaaacctaaagatactttaatgataagtcgtaccccagaagtcacgtgtgtcgttgttgatgtttcacacgaggatcctgaagtgaaatttaattggtatgttgacggcgtcgaagtacataatgcaaaaacaaaaccaagagaagagcagtatgcttctacatacagggtggtaagtgtgctaaccgtactacaccaagattggttaaacggtaaggaatataaatgtaaagtttctaacaaggctctgccagctcccatagaaaagactatttccaaggccaagggacaacccagagaaccccaagtatatacccttccaccttctagggaagagatgaccaagaatcaggtgtcactaacctgtctagtcaagggcttttatccttctgatatagctgtagagtgggaatctaatggccagcctgagaacaattacaaaactaccccacccgtgcttgatagtgatggatcattcttcctttactcaaagctgaccgtggacaaaagtaggtggcagcagggtaacgtcttctcctgcagtgtaatgcatgaagccttagcaaatcattatacacaaaaatccttatcactgtcacctggctgt

SEQ ID NO: 330 - DNA, 732 bp, Artificial Sequence
  Description of Artificial Sequence Synthetic polynucleotide
caggttcagctggtcgagtccggcggtggactggtccaaccaggtggatctcttacactgagttgtacggcctctggatttacgctggaccactacgacattggatggtttaggcaagcaccaggtaaagagagggagggtgtttcctgcataaataattccgatgatgatacttactatgctgactctgtgaaaggtaggttcacgatctttatgaacaatgccaaagacactgtgtacttacaaatgaactcattgaaaccagaggataccgctatttactactgcgctgaagcccgtggctgtaagaggggtcgttacgagtatgacttctggggacagggaactcaagtaacggtctcatcagaacccaagtcttctgataagacgcacacatgccccccttgtcccggacagccaagggagccacaagtctacacattacccccatccagagaagagatgaccaagaatcaagtatccttaacctgcttggttaagggattttacccttcagatatagcagtagaatgggaatctaatggccaaccagaaaacaactataaaaccacgccaccagttctggactccgatggttccttcttcctatacagtaaattgaccgttgataaatccagatggcaacaaggcaacgtgttcagttgctcagttatgcatgaggctttgcataatcattatactcagaaatctttgtccctttcccccggatgc

SEQ ID NO: 331 - DNA, 738 bp, Artificial Sequence
  Description of Artificial Sequence Synthetic polynucleotide
caagttaaactggaagagtcaggtggcggttcagtacagaccggtggttcacttcgtctaacttgtgcagcaagtggaagaacctcccgttcttacggtatgggttggtttcgtcaagcacccggtaaggaacgtgaatttgttagtggtataagttggagaggcgactctacgggctatgccgatagtgtcaaaggtaggttcactatttctagagataatgctaaaaacaccgtcgacttgcaaatgaactcattgaaaccagaagatacggctatatattactgtgctgccgccgctggatcagcatggtacggcacactgtacgaatatgactactggggccaaggcacgcaggtaaccgtgtccagtgaacccaagtcatccgacaaaacccacacctgtcctccatgccccggccaaccaagggaaccacaagtatatacgttacctccaagtcgtgaggagatgactaagaaccaggtttccctgacctgcctggttaaaggtttctacccaagtgacatcgcagttgaatgggaatccaatggtcagcctgagaacaattataagactacccctcctgtcctagatagtgacggttcatttttcttatactctaaattgaccgtagataaaagtagatggcaacaaggcaatgttttttcatgctctgtaatgcacgaggctcttcacaaccactacacacaaaagagtctttctctgtccccaggctgt

SEQ ID NO: 332 - DNA, 702 bp, Artificial Sequence
  Description of Artificial Sequence Synthetic polynucleotide
caactacaattagtggaatctggaggcggtcttgtgcaacccggcggttccttaaaattgtcctgcgctgcctcaggatttacttttagtagatatgccatgtcatggtacaggcaggctccaggcaaggaacgtgaaagtgtggcaagaatatctagtggaggaggaacgatatattacgcagattccgtaaaaggcagatttactattagtcgtgaagatgcaaaaaatacagtataccttcagatgaactctttgaaaccagaagatacggccgtttattattgttacgttggaggattttggggacagggaacccaggtcacagtaagttccgagcctaaatctagtgataaaacgcatacttgtccaccttgtccaggccagcctcgtgagccccaagtttatacgcttcctccctctcgtgaagaaatgacaaaaaatcaggtctctctaacctgccttgtaaagggattttacccatctgacattgccgtcgaatgggaaagtaacggacagcccgagaataattataaaacgacacctcccgtcttagattctgatggctctttctttttatacagtaagctgactgtggacaagagtagatggcaacagggtaatgttttctcttgttcagtcatgcatgaagccttgcataaccattatacccaaaaatctcttagtctttctcccggatgt

SEQ ID NO: 333 - DNA, 738 bp, Artificial Sequence
  Description of Artificial Sequence Synthetic polynucleotide
gaggtacagttagtggagtctggtggtggctccgtacagacaggaggatcattaaggcttagttgtgccgtatccggcaatataggttcatcctattgtatgggatggttccgtcaagcacccggtaagaaaagggaggcagtggcaagaatagtaagggacggtgcaaccggctacgcagattacgttaagggaaggtttacgattagtagagatagtgcaaaaaatactctataccttcaaatgaataggctaattcccgaggatacagctatatactattgcgccgcagacctacccccaggctgtttaactcaggctatatggaacttcggatatcgtggccaaggaaccctggtcacggtaagttcagaaccaaaatcctctgataaaacccatacttgtccaccttgccctggtcaaccaagagagccccaggtatatacgctgcccccttctcgtgaagaaatgacgaaaaatcaggtgagtctaacgtgtttagtaaagggattttacccctccgatatagccgttgagtgggaaagtaatggtcagccagaaaataactataaaacgacaccccctgtgttagactcagacggatctttctttttatattcaaaacttactgtagataagtcccgttggcagcaaggtaatgtattctcatgttcagtgatgcatgaggccctgcataaccattatacccaaaagagtctatccctgtctcccggctgc

SEQ ID NO: 334 - DNA, 711 bp, Artificial Sequence
  Description of Artificial Sequence Synthetic polynucleotide
gaggtccagcttcttgaatccggcggaggtctggtgcagcccggtggctctcttcgtctaagttgcgccgcatcaggctttactttctctactttttctatgaactgggtaagacaagccccaggtaagggtctagagtgggtttcttatatttctcgtacctctaaaacgatatattatgctgattcagttaagggacgttttacgatatctagggacaacagtaaaaataccttgtatcttcaaatgaactctctgcgtgcagaagataccgcagtgtattactgcgctagaggccgtttttttgattactggggtcaaggaacacttgttaccgtatctagtgagccaaagtcttccgacaaaacacatacatgtcctccctgccccggacagcctcgtgaaccacaggtatatacgcttccaccaagtagagaggaaatgactaagaatcaggtttcactgacatgcctagtgaagggcttttacccttctgatatagctgtggagtgggaatcaaacggccagcccgagaacaattacaaaacgactccacctgtgctagacagtgacggctccttttttttatactcaaaactaacggtagacaagtctaggtggcagcagggcaatgttttcagttgtagtgtcatgcacgaggcactacataatcattacacacaaaagtcactgtctctatcccccggttgt

SEQ ID NO: 335 - DNA, 510 bp, Artificial Sequence
  Description of Artificial Sequence Synthetic polynucleotide
tgccaggtccaactggtggagtccggcggtggtctagtccaaccaggaggttcattgaccctttcatgtaccgcatcaggtttcacgcttgaccactacgacatcggctggtttcgtcaagccccaggcaaagagagagaaggtgtttcctgtataaataattccgatgacgacacatactacgctgactccgtgaagggcaggttcaccatattcatgaataatgctaaagataccgtgtacttgcaaatgaacagtttgaagccagaagacacagccatctattattgcgccgaagcccgtggttgtaaacgtggtaggtatgagtacgatttctggggtcagggtacacaggtaacagtctcctcaggaggaggcggttcattgcctgtctacacattgaaggaaaggtgcctacaagttgtccgtagtctagttaagcctgagaattataggcgtctggatattgtgaggtccctgtatgaagaccttgaggaccatcctaacgtc

SEQ ID NO: 336 - DNA, 510 bp, Artificial Sequence
  Description of Artificial Sequence Synthetic polynucleotide
ttaccagtctatactctaaaagaaaggtgcctacaagttgttaggtccctggtaaagcctgagaactataggaggttagacatcgtgcgttccctgtatgaggacttggaggatcacccaaatgtgggcggtggtggctcacaagtacagctagttgaaagtggtggtggacttgtacagcctggtggctccttgactttatcctgtacagcatcaggttttacgttggaccactatgacatcggatggttccgtcaagccccaggaaaagagagagaaggcgtgtcatgcatcaacaattcagatgacgatacttactatgctgactctgtgaaaggaaggtttactatttttatgaataatgctaaagatacggtgtacttgcagatgaactctcttaaacctgaagatacagcaatttattattgtgctgaagcaaggggatgcaagagaggcaggtacgaatacgatttctggggacaaggcacccaagttaccgtttcatcctgc

SEQ ID NO: 337 - DNA, 516 bp, Artificial Sequence
  Description of Artificial Sequence Synthetic polynucleotide
tgccaggtcaaactggaagagtcaggcggcggttctgttcaaactggtggaagtcttaggttaacgtgtgccgcatctggcaggacatccagaagttatggcatgggatggtttcgtcaggctccaggaaaggaaagggagtttgtgagtggtatatcttggaggggcgatagtacgggatacgcagactcagtgaagggaagatttaccatatcccgtgacaatgctaagaataccgtagatctgcaaatgaactccctgaagccagaggatacagccatatattactgtgctgcagccgccggatcagcttggtatggaacactatatgagtatgattactggggccaaggaacccaagtgactgtttcatctggtggcggaggttccctaccagtatataccctgaaagaaagatgtctacaagtcgtcagatccttggtcaaacccgagaattatcgtaggttggatattgtaagaagtctatacgaggaccttgaagaccaccccaatgtt

SEQ ID NO: 338 - DNA, 516 bp, Artificial Sequence
  Description of Artificial Sequence Synthetic polynucleotide
ttgcctgtctacactctgaaggagaggtgccttcaggtagtcaggtcccttgtcaaacccgaaaattatcgtaggctggatatagtccgttccctatatgaagacctagaagaccaccccaatgtaggtggtggaggatcacaagtgaaactggaagaaagtggtggaggcagtgtccaaacaggaggatcactgcgtctaacgtgcgctgctagtggcagaacttcacgttcctatggaatgggctggttcaggcaagcaccaggcaaggagcgtgaatttgtctctggaatctcctggcgtggcgattccacaggctatgcagactcagtaaagggcaggtttacaatttcaagggacaacgccaaaaacactgtcgatcttcagatgaacagtctaaaacccgaagacacagcaatatactactgcgccgccgccgccggaagtgcctggtatggcacgctttatgaatatgattactggggtcaaggaacccaggtcactgtttccagttgc

SEQ ID NO: 339 - DNA, 480 bp, Artificial Sequence
  Description of Artificial Sequence Synthetic polynucleotide
ttgccagtatataccctgaaggaaagatgtcttcaagtagtaagatcacttgttaagcccgaaaactatagaagattggatattgtacgtagtctatatgaagatttggaggaccatccaaatgtcggtggaggtggtagtcagttgcagctggtggagtcaggtggtggcctggtccaaccaggcggatcattaaaattatcctgcgctgcaagtggctttacgttcagtagatatgctatgagttggtacaggcaagctccaggaaaggaaagagaatctgtagcacgtatatcttcaggcggaggcacgatctattatgccgactccgttaagggccgttttaccatatcccgtgaagacgctaaaaatacagtatatctgcaaatgaactctttgaagccagaggatactgccgtttactattgctacgtaggcggtttctggggccaaggtactcaggttacggtgagttcctgc

SEQ ID NO: 340 - DNA, 666 bp, Artificial Sequence
  Description of Artificial Sequence Synthetic polynucleotide
tgtcaagtccaacttcaagaaagtggtggtggcctggtacaaccaggaggatctttaaagctgagttgtgctgcatccggttttactttctcacgttacgcaatgagttggtatagacaggctcccggcaaggaaagggagtctgtggccaggatatcaagtggaggtggcaccatctactacgctgactctgtgaagggaagattcactatctcaagggaggacgcaaagaacacggtttacttgcaaatgaatagtctgaaacctgaagacacagccgtttattactgttatgtgggcggattctggggacagggaacgcaagtaacagttagttccggtggcggcggctctcatagttataggggccatttatggttgttcagggacgcaggtacacatgacggactgctagtagcccagacagagctatttgttccatcccttaatgtggatggacagccaatctttgccgctataaccttacctgtttatacgttgaaggaaagatgtctacaagtggtaagatctttggtaaagcccgagaactatagaaggctggacattgtccgttccctgtacgaagacttggaggatcatcccaacgtacaaaaagacctagaaaggctaacccaagagaggattgcccatcagagaatgggtgat

SEQ ID NO: 341 - DNA, 582 bp, Artificial Sequence
  Description of Artificial Sequence Synthetic polynucleotide
tgccaggttcagctacaggagtccggcggtggcctagttcaaccaggtggttcattgaaattaagttgcgccgccagtggctttaccttctccaggtacgccatgtcatggtatagacaggcacctggtaaggagagagaatcagttgctaggatatcttcaggaggcggtacgatttattatgccgactcagttaagggacgtttcacaatatcccgtgaggacgcaaaaaatacggtctatttacaaatgaactccctgaagcccgaggatacagccgtttattactgctacgttggaggcttctggggacaaggcacacaggtaacggttagttctggcggaggaggttcaccctcattaaacgtggatggtcaacccattttcgccgctatcaccttgcctgtgtacacgctgaaggagagatgcttgcaggtcgtgaggagtcttgttaagcctgaaaactatagaaggttagatattgtcagatccctatatgaagacttagaggatcatcccaacgttcaaaaagacctggagagactaacccaagaaagaatagctcatcaacgtatgggagat

SEQ ID NO: 342 - DNA, 540 bp, Artificial Sequence
  Description of Artificial Sequence Synthetic polynucleotide
tgccaagttcagctacaagaatcaggcggaggtcttgtacaaccaggaggctctctaaaactttcatgtgccgcatctggtttcaccttctcccgttatgcaatgagttggtacagacaagctcccggaaaagagagagaatcagtggcaagaatttcaagtggcggtggaacgatatattatgccgattccgtaaaaggcaggttcaccatatcaagggaggacgccaagaatacagtatatctgcaaatgaactccctaaaacccgaagatacggctgtgtactattgctacgtgggcggtttttggggtcaaggaacccaggttaccgttagttctggtggcggtggtagtacgctgcccgtttatacactaaaagaaagatgcctacaagtagtacgttcacttgttaaaccagagaactacaggcgtttagacatcgttaggagtctttatgaagacctagaagatcatccaaacgtccaaaaagatttagaaaggctgacacaagaacgtatagctcaccagagaatgggtgat

SEQ ID NO: 343 - DNA, 507 bp, Artificial Sequence
  Description of Artificial Sequence Synthetic polynucleotide
tgtcaggtacagctagtggaaagtggcggcggactggttcagcctggcggttccttgacactatcctgtacggctagtggattcactttggatcactacgatataggctggttcaggcaagcccctggtaaggaacgtgaaggcgtctcttgtatcaataactcagacgatgatacatattacgccgactcagtgaaaggaaggtttacaatctttatgaataacgccaaggatacagtctatcttcaaatgaatagtcttaagcctgaggacaccgctatatactattgcgccgaggccagaggttgcaaaaggggtagatatgagtacgacttttggggtcagggaacccaggttacggtctcctctggtggtggcggttcagtcagacctctgcaagaattatgcaggcaaagaatcgtggccactgtcggtcgtgaaaatttggcccgtatccctctaaaccccgtcctgcgtgactacctttcatcattcccattccaaatt

SEQ ID NO: 344 - DNA, 507 bp, Artificial Sequence
  Description of Artificial Sequence Synthetic polynucleotide
gttcgtcctttacaggaattatgcaggcaacgtatcgtcgccactgtgggacgtgagaatctagccaggattccccttaatccagtcttgagggattatctatcatcatttccattccagataggtggtggaggttcccaggttcaattggtagaaagtggtggtggacttgtccaaccaggtggttccctaaccctaagttgcactgcctctggattcacgctggaccattacgacatcggctggtttagacaagcacctggaaaagagagagaaggcgtctcctgcataaacaactccgatgacgatacatattacgctgattcagtcaagggtcgtttcactatcttcatgaataatgccaaggacactgtctaccttcaaatgaatagtttgaagcctgaagacacagccatttattattgtgcagaagcaaggggctgtaagaggggcaggtatgaatatgatttttggggtcagggcacgcaggtgaccgttagtagttgc

SEQ ID NO: 345 - DNA, 513 bp, Artificial Sequence
  Description of Artificial Sequence Synthetic polynucleotide
tgtcaagtcaagttagaggaatctggcggaggatcagtccagacgggcggcagtttgaggcttacctgtgcagcatccggtagaacgtcaaggtcctacggtatgggatggtttcgtcaggccccaggaaaggagagggagtttgtgagtggtatatcatggaggggcgactcaactggctatgcagattctgtaaagggtcgttttacaatctccagagataacgccaaaaacacggtcgacctgcagatgaacagtctgaaacctgaagacacagctatctactattgtgccgccgcagccggatctgcttggtatggaactttatacgaatatgactattggggccaaggcacacaagtcaccgtttctagtggcggtggcggaagtgtgcgtcccctacaggaactttgtagacagagaatcgtcgctaccgtaggcagagaaaacttggctaggatccctttgaacccagtcttacgtgactatctgtcatccttcccatttcaaatc

SEQ ID NO: 346 - DNA, 513 bp, Artificial Sequence
  Description of Artificial Sequence Synthetic polynucleotide
gtacgtccattgcaggagttatgtcgtcaacgtatcgtggcaaccgtaggcagagagaacttggcccgtatccctctaaaccccgtattacgtgattatctttcatctttccctttccagatcggaggcggtggatctcaggttaagctggaagaatcaggaggaggaagtgtacagactggaggttctttacgtttaacgtgtgctgctagtggtcgtacatcacgttcatacggcatgggttggttcagacaagcccctggcaaggaaagagaatttgtatcaggtatctcatggagaggtgacagtacgggctacgccgactctgtaaaaggcaggtttacgatttccagggataacgcaaagaatactgttgacttacagatgaactctttgaaacctgaagacaccgctatctattattgcgcagccgctgccggtagtgcatggtatggcaccctttatgaatatgactattggggccagggtacacaggtaacggtcagttcctgc

SEQ ID NO: 347 - DNA, 477 bp, Artificial Sequence
  Description of Artificial Sequence Synthetic polynucleotide
tgccagcttcaacttgtcgaaagtggtggaggccttgtgcaaccaggaggtagtctaaaattgtcatgtgctgcatcaggcttcacattctctagatacgccatgtcttggtatcgtcaagctcctggtaaggaacgtgaatccgtagctcgtatctcttcaggtggtggcaccatttactacgccgattctgtgaaaggcagatttacgatttctcgtgaggatgccaagaacacggtctaccttcagatgaactccctaaaacctgaagacacggcagtctactattgttacgtgggtggcttttggggtcaaggtactcaggttacagtatcatcaggcggcggcggatctgtgagaccactacaggagttatgtcgtcagcgtatagtcgctaccgtaggacgtgaaaatttagctcgtattcccctgaaccccgtcctacgtgattacctatcctcatttcccttccaaata

SEQ ID NO: 348 - DNA, 477 bp, Artificial Sequence
  Description of Artificial Sequence Synthetic polynucleotide
gtccgtcctcttcaagagttatgcaggcaacgtatcgttgctacagtaggaagagagaacttagcccgtatcccactgaaccctgtcttgagggactatctttcttctttcccattccagataggaggtggcggatcacagctacaacttgtcgagtctggtggtggcctagtacaaccaggtggctccctgaaactgtcctgcgctgccagtggttttacttttagtcgttatgctatgagttggtacaggcaggcacccggcaaggaaagagagtcagtcgctaggatttcctctggcggtggtacgatatattatgccgatagtgtcaagggtaggttcaccataagtcgtgaggacgctaaaaacacagtgtatcttcaaatgaactcactaaaaccagaggacacagctgtttattactgctacgtaggtggcttttggggtcaaggtacacaagtcacggtcagttcctgt

SEQ ID NO: 349 - DNA, 1164 bp, Artificial Sequence
  Description of Artificial Sequence Synthetic polynucleotide
gacttgggtaaaaagctgcttgaggcagctagagcaggacaagatgacgaggtaagaatactaatggcaaatggagcagatgtcaatgcctccgatagatggggatggaccccattacatctggcagcatggtggggacaccttgaaattgtcgaagtgcttttaaaacgtggcgctgatgtctctgctgctgatttgcacggccagagtcctctacacctagctgctatggtcggccatctagagattgtggaagtattactgaagtatggtgccgacgtaaatgccaaagacaccatgggagcaacacctcttcacctggcagctagaagtggccacttagaaatcgtagaagagttgctgaagaatggtgccgacatgaatgcccaggataagttcggaaagactactttcgacatctctacagataacggcaacgaggacttagcagagattctacaaaaacttggtggaggcggatcagacaagacgcacacgtgtcctccctgtcctgctcctgaattgttgggcggaccatccgtttttctttttccccccaagcctaaagacactcttatgatctctcgtacccctgaagttacttgcgtagttgtcgatgtatcacatgaggaccctgaggtgaaatttaactggtatgttgacggagtagaggttcacaacgctaagacaaaaccacgtgaggaacaatatgccagtacataccgtgtcgtatctgtgctaactgtactacatcaagactggcttaatggcaaagaatataaatgcaaggtgtcaaacaaggcacttccagcccccattgaaaaaactattagtaaagccaagggtcaacctcgtgagccccaagtctacacactaccaccttcacgtgaagagatgacaaaaaaccaggtatccttaacctgtttggtcaaaggcttctatccttcagatattgcagttgagtgggaatctaacggccagcctgagaataactacaaaactacaccccctgtcttggattccgacggtagtttcttcctatacagtaaactaactgtagataagagtaggtggcagcagggcaatgtcttctcctgctccgtgatgcacgaagctctacacaatcattatacccaaaagtcattgtcattgtcacccggttgc

SEQ ID NO: 350 - DNA, 1164 bp, Artificial Sequence
  Description of Artificial Sequence Synthetic polynucleotide
gatttgggtaagaaattgttagaggccgcaagggcaggacaagatgatgaggttcgtatcttaatggcaaatggtgccgacgtgaatgcatccgacagatggggctggactcctctgcatttagccgcttggtggggccacctggaaatagttgaagtattgttgaagagaggtgctgatgtttcagccgccgatttgcatggacagtcccccttacatcttgctgcaatggtcggtcatctagaaatcgtagaggttctattgaaatacggagcagatgtcaacgccaaggatacgatgggagctactcctctacacttggccgcaagaagtggccatttggaaatagtcgaggagttattgaagaatggagcagatatgaatgcccaggacaagttcggaaaaacaactttcgacatatctacagataacggaaatgaggatttggctgaaatactgcaaaaactaggtggaggcggctcagataaaactcacacatgtcccccttgccccgcccctgagttacttggaggaccatctgttttcctgtttcctcccaaaccaaaagacacccttatgatttctaggacacccgaggtaacatgtgtggttgttgacgtttcacatgaagaccccgaggtgaaattcaattggtatgtagacggcgtagaggtccataatgccaaaacaaaacctcgtgaagagcaatacgcctccacatatagagttgtatctgtgctaaccgtcttacatcaagattggctaaatggtaaagagtataagtgcaaagtgtccaacaaggccctacctgccccaattgaaaaaacgatatctaaagcaaaaggtcagcctagagaaccacaggtctatacattacctccttccagagaagaaatgacgaagaaccaggtaagtttaacttgtctagtaaaaggtttttacccatctgacattgctgtagaatgggaaagtaatggtcaacctgagaataattacaaaacgacaccacctgttctggattccgatggctccttttttttgtattccaagttaactgttgataaatcacgttggcaacaaggcaatgtcttttcctgttcagttatgcacgaggcacttgctaatcactacacacagaagtctctatctctgtcccctggttgt

SEQ ID NO: 351 - DNA, 1194 bp, Artificial Sequence
  Description of Artificial Sequence Synthetic polynucleotide
gacctaggcaaaaagttattggaggccgcaagagctggccaagatgacgaagtgcgtattttaatggccaacggagctgacgtcaacgccagtgataggtggggttggaccccccttcatcttgccgcctggtggggccacctggagatcgtcgaagtacttttaaagagaggagctgatgtcagtgctgctgatttgcatggccaatcccctcttcatttggccgcaatggtgggacacttagaaatagtagaagtcttacttaaatatggagcagacgtcaatgcaaaggacacgatgggagctacaccattgcacttagcagcacgttcaggacatcttgagattgttgaagaactgctgaaaaacggtgcagacatgaatgcccaggataagttcggtaagactacattcgacatttctacagacaacggaaatgaggatctggccgagatacttcaaaaactgggcggaggtggtagtggcggtggcggttccgagcccaagtcatcagacaaaacacacacttgtccaccctgtccagccccagagttacttggaggcccttgcgttttcttattcccaccaaagcccaaagataccctgatgataagtagaacccccgaagtcacatgcgtagttgttgatgtgtcacacgaggaccccgaagtgaagttcaactggtacgtcgacggtgtcgaggtgcataatgctaaaactaagccccgtgaagagcaatacgcttccacgtatagggtcgtttctgtgcttacagtgttacaccaagattggctgaatggaaaggagtataaatgcaaagtctcaaacaaggctcttcccgccccaatcgagaaaactatatctaaagcaaaaggtcaacctcgtgagcctcaagtctacactctacctccttctagagacgagctgactaagaaccaggtgtcactgacgtgccttgtgaaaggattctatcccagtgatatcgcagtagagtgggagtctaatggacaacccgaaaataactataagaccacacccccagtactggactcagacggcagtttcttcttatacagtaagttgaccgttgacaaatctcgttggcagcaaggaaacgtcttctcttgcagtgtcatgcatgaggctctgcataaccactacacgcaaaagtccttatctttgtcccccggcaaa

SEQ ID NO: 352 - DNA, 1176 bp, Artificial Sequence
  Description of Artificial Sequence Synthetic polynucleotide
gataagacccacacgtgcccaccctgccctgcccccgaacttcttggtggaccttgtgtatttctattccctcccaagcccaaggatactttaatgatttccaggacccctgaggtgacatgcgttgtagttgacgtatcacatgaagacccagaagtcaaatttaactggtatgtagacggcgtggaggtgcataacgccaaaacaaagcctagggaggaacaatatgcatcaacgtacagggtcgtctccgtactaacggtgttacatcaagactggctaaatggtaaggaatacaaatgtaaagtgagtaacaaggccctacccgcccctatcgagaaaacaatctccaaggcaaagggtcaacctagagaaccccaggtatatacgcttcccccttccagggatgagttaaccaagaatcaagtaagtttgacatgcttggttaagggcttctacccctccgatattgccgtggaatgggagtccaatggccagcccgagaataattataagactacccctccagtcctggattcagacggctcattttttctttactcaaagctaacggtggacaagagtagatggcagcagggaaatgtcttttcatgctcagtaatgcatgaagcattacataaccactatacgcaaaaatccctttctcttagtcccggtggaggtggaggatcaggaggcggcggctccgatttgggtaaaaaattgttagaggctgccagagccggacaggatgatgaagtcaggatcttgatggcaaatggtgcagacgtgaatgcttcagatagatggggatggaccccactacatctagccgcatggtggggccacctggaaattgttgaagtacttttaaagcgtggtgccgacgtgtcagctgcagatctacacggtcaatcacccttgcaccttgcagccatggtgggtcatttagagattgtcgaagttcttcttaaatacggagctgacgtaaatgctaaggacactatgggtgctactcccttacatctagctgcaaggtccggccatttggagatcgtcgaggagctattaaagaacggtgctgacatgaatgcacaggataaatttggaaaaacgacgtttgatatatccactgacaacggcaatgaagacttagctgagatcctgcaaaagtta

SEQ ID NO: 353 - DNA, 849 bp, Artificial Sequence
  Description of Artificial Sequence Synthetic polynucleotide
gacctaggaaaaaagttgcttgaggccgctagggccggacaagatgatgaagtgcgtatcttaatggctaacggtgccgatgtcaatgccagtgacaggtggggatggactcctttgcacttggcagcatggtggggtcatttggaaattgtagaggtgcttttgaagaggggtgccgacgtttctgctgctgatcttcacggtcagagtccccttcaccttgctgctatggttggtcatttagaaattgtggaagttctattaaagtacggcgcagatgtcaatgctaaagacacaatgggtgctactccattgcatttagctgctaggtctggacacttagaaatagtagaagagttgcttaagaatggcgctgacatgaacgcccaagacaagtttggaaaaaccacgtttgacattagtacggataatggcaacgaagacttggcagaaatcttacagaagctgggaggaggtggtagtgaacctaaatcttcagataagacccatacttgccccccatgtcctggacaacccagggagcctcaagtgtatactttgcccccttctagggaagagatgaccaaaaaccaagtctccttgacgtgtttggtaaaaggcttttacccttccgatatcgccgttgaatgggagagtaacggacaacccgaaaacaattacaagacgaccccaccagtgcttgactctgatggtagtttttttttgtacagtaaactgacggttgataagtctcgttggcaacagggcaatgttttttcttgttcagttatgcatgaagccttgcataaccactatacgcaaaaatcactgtctctttcccccggctgt

SEQ ID NO: 354 - DNA, 873 bp, Artificial Sequence
  Description of Artificial Sequence Synthetic polynucleotide
tgtgaccttggtaagaaactactagaggcagccagggctggccaggatgacgaagtgcgtattttgatggcaaatggagctgatgtaaacgcctctgatagatggggatggacaccccttcatcttgctgcctggtggggacaccttgagatcgtcgaagtgttattgaaaaggggagcagatgtgagtgccgccgatctgcatggccaaagtccattgcatttagccgcaatggtaggacatttagagatagtggaggtacttctgaagtatggagcagatgttaatgccaaagacacaatgggtgctaccccccttcatttggctgcaagatcaggccatctagaaatagttgaggagttattaaagaatggtgccgatatgaatgcacaggacaagtttggtaaaactacttttgatatctcaactgataatggtaacgaggatttggcagagatattacaaaagctaggaggtggaggatctggaggaggcggttctcaggttaagctagaggagagtggcggaggttccgtacaaactggaggctcattgcgtttgacttgtgcagcatcaggtaggacatcccgttcatacggaatgggctggtttagacaggcccccggtaaagaacgtgagttcgtttctggtatatcatggagaggcgactctaccggatatgctgattcagtcaagggtagatttaccatctcacgtgataatgctaaaaacacggttgacttgcaaatgaactcattgaaacctgaggatacagcaatatactactgcgcagccgcagccggatcagcatggtatggtaccctgtacgaatacgattactggggacagggcacccaggtcactgtatcctct

SEQ ID NO: 355 - DNA, 873 bp, Artificial Sequence
  Description of Artificial Sequence Synthetic polynucleotide
gaccttggcaagaagttgctagaagctgcaagagccggccaggacgacgaggttagaatacttatggccaacggtgcagatgttaatgctagtgatagatggggctggacacctttacatctagctgcttggtggggacatcttgaaatcgtcgaggtgttgctaaagaggggagctgatgtgtcagccgcagacctgcatggtcaatctccactacacctggcagctatggtaggtcacttagagatagtcgaggttctgttgaaatacggcgcagatgtaaatgctaaagatacgatgggtgccacacctttacacttagcagctaggtccggtcacctggaaatagtagaagaactgctaaagaacggagctgatatgaatgctcaggacaaatttggaaagactacttttgatattagtacagacaacggtaatgaagacctagcagaaatcttgcaaaaactgggcggcggtggtagtggaggcggaggttcacaggttaagttggaggaatctggcggcggttccgtccagaccggcggcagtttaagattgacatgtgcagcatctggtcgtacttccagaagttatggcatgggctggttcaggcaggctcctggtaaggaaagagagtttgtgtctggtatctcatggaggggcgactccaccggctatgctgactcagtgaaaggccgtttcacaatctctagagataacgcaaagaataccgtcgacctgcagatgaacagtttgaagcccgaggatacggctatatactactgcgccgccgcagccggttccgcttggtacggaaccttgtacgaatatgattactggggccaaggcacacaggtcacagtctcctcctgc

SEQ ID NO: 356 - DNA, 873 bp, Artificial Sequence
  Description of Artificial Sequence Synthetic polynucleotide
tgccaggtgaaattagaggaatctggaggtggctccgtgcagacaggtggaagtctgcgtcttacttgtgctgcatcaggacgtacgagtagaagttatggaatgggatggtttagacaggctcccggtaaggagagagagtttgtcagtggaatttcctggaggggcgattccacgggatacgctgattcagttaaaggaagattcaccatctctcgtgacaacgcaaaaaacacagtggatttacagatgaatagtctgaagcctgaggataccgccatatattactgcgccgcagcagcaggaagtgcttggtacggaacattatacgagtacgactactggggccaaggtacgcaagttactgtgtcttctggtggaggcggttccggtggaggtggcagtgacttaggcaaaaagttattggaggccgcaagagccggtcaagacgacgaagtgaggatacttatggcaaacggtgccgacgtgaatgcatccgacaggtggggctggacccctcttcacctagccgcttggtggggacatcttgaaatcgtagaggtgctattaaagcgtggagccgacgtctcagcagcagatttacacggacagtctcctctacatctagcagctatggtgggtcacttggaaattgtggaggtgttactaaagtatggtgcagatgtaaacgccaaagatacaatgggcgcaacgccattgcatcttgccgctagatccggccatttggagatagtcgaagagctattgaaaaacggcgctgatatgaacgcccaagacaagttcggaaaaactacatttgatatatctacagacaacggtaacgaagacttggccgagattcttcagaagtta

SEQ ID NO: 357 - DNA, 873 bp, Artificial Sequence
  Description of Artificial Sequence Synthetic polynucleotide
caggttaagcttgaagaatccggcggtggatcagttcaaactggcggaagtttgcgtcttacttgtgcagcttcaggcagaaccagtcgtagttacggaatgggttggttccgtcaggcacctggaaaagaaagagagttcgtgagtggaatttcatggcgtggcgattctacaggctacgctgacagtgtaaagggacgtttcacgatctcacgtgataacgcaaaaaatacagttgacttgcaaatgaactccttaaagccagaggacaccgctatatattattgtgccgcagcagcaggttctgcatggtatggaaccttatacgaatatgactattggggtcaaggaacccaagttactgtcagttctggtggaggcggcagtggaggtggaggctccgacttgggaaaaaaactgttggaggcagcaagagcaggacaagatgacgaggttcgtatacttatggctaatggagccgacgtgaacgcaagtgataggtggggttggacacccctacacctagctgcctggtggggacacctagaaatcgtggaagttctactaaaaaggggcgccgacgtttcagccgcagacctacacggtcaatccccattgcatctagcagctatggtaggacatcttgagattgtagaagtgctactaaagtacggtgccgatgtaaatgccaaagacactatgggagcaacgccattacatctagccgcaaggtcaggtcatttagaaattgtcgaggagttactaaaaaatggcgctgatatgaacgctcaagataaattcggtaaaacgacttttgacatatcaacagataatggaaacgaagaccttgccgaaattttgcagaagctttgc

SEQ ID NO: 358 - DNA, 1074 bp, Artificial Sequence
  Description of Artificial Sequence Synthetic polynucleotide
tgtcaggtgaaattagaagaatccggcggtggtagtgtgcagaccggcggaagtctacgtcttacctgcgctgccagtggtaggacatcccgttcctacggtatgggctggtttcgtcaggctccaggaaaagaaagagagtttgtaagtggtatatcctggcgtggcgattccaccggatacgccgactcagtcaaaggccgtttcacaatctccagagataacgccaaaaatacggttgacctacagatgaacagtctaaagcccgaagacacggcaatttactattgtgcagccgccgccggatccgcctggtatggaactctgtatgagtacgactactggggacaaggcacccaggtgacagtgagttctggaggaggcggcagtggtggtggaggatcagacctgggaaagaagttactggaagcagctagagccggacaggacgatgaagtcaggatattgatggctaacggtgctgacgtcaacgcaagtgacagatggggctggacgcctttgcatctagctgcatggtggggccatttggagatagtggaggtacttcttaaaagaggtgccgacgtgtccgctgctgatcttcatggccaatctccactacatttagcagcaatggtcggacatttggaaatcgtcgaagtcttgctgaagtatggagcagatgtcaatgcaaaagacacaatgggagctactccccttcacttagccgccaggagtggccacttagagattgtggaggaactacttaaaaatggtgccgatatgaacgcccaagacaagttcggtaagacgaccttcgacatctctaccgataacggtaatgaagaccttgcagaaatactgcagaaactaggaggtggaggtagtactttgcctgtatatacgttaaaagagaggtgcttgcaggtagtacgttctctagtgaagccagaaaattacaggcgtttggatatcgttagatccctgtatgaggatctggaggatcaccccaatgtgcagaaggacttggagagattaacccaagagaggatcgcacatcaaaggatgggcgat

SEQ ID NO: 359 - DNA, 1014 bp, Artificial Sequence
  Description of Artificial Sequence Synthetic polynucleotide
tgtcaggttaagttggaagaatcaggcggtggcagtgtccagacgggtggatcactgaggcttacgtgcgcagcatccggaagaacgagtaggtcttacggaatgggatggtttaggcaagctcctggtaaggagcgtgagttcgtatcaggtatatcatggcgtggagattctacaggctatgcagactctgtcaaaggcaggttcactatatctagggacaacgctaaaaatacggtcgacctacaaatgaatagtcttaagcccgaggacactgcaatttactactgcgctgccgctgcaggctctgcatggtatggtactctgtacgagtacgattactggggccaaggaactcaagtcacagtatcttcaggaggtggaggttctggaggaggcggctcagatctgggcaagaaactgttagaagccgcaagagctggacaagacgacgaagtaagaatcctaatggcaaacggagccgacgtaaatgcatccgatcgttggggctggacaccattacacttggctgcttggtggggacacttagagattgtcgaggtattactgaagaggggagcagatgtatccgcagcagatctgcacggtcaatccccactacacttagccgcaatggtgggccatctagaaattgtggaagtgcttttgaaatatggtgctgatgttaatgccaaggatacgatgggcgccacgccactacatctagccgcaaggtcaggtcacctggaaatcgtcgaagaactgttaaaaaacggcgctgatatgaatgcccaggataagttcggtaagaccaccttcgacataagtactgataatggtaacgaagacctggctgaaattttgcaaaagttgggaggtggtggcagtttgcccgtttatacactgaaagaaaggtgtttacaggtggtaagatccttggtgaagccagagaattaccgtagattggatatagttaggtctctgtatgaggacttggaagatcaccctaacgtg

SEQ ID NO: 360 - DNA, 1074 bp, Artificial Sequence
  Description of Artificial Sequence Synthetic polynucleotide
tgtgatctgggaaaaaagcttcttgaagctgccagagctggccaagatgatgaggtgcgtattcttatggcaaacggagctgatgtcaatgccagtgacaggtggggatggacgcctctgcacttggcagcatggtggggtcacctagagatagtagaggttcttctaaagaggggtgcagatgtttctgctgccgacttacatggacagtcacctcttcacctggcagccatggttggccatttggagatagttgaggtcctattaaaatacggcgccgatgtgaatgctaaagataccatgggcgccacgccattgcatcttgccgccagatctggccatcttgaaatcgtcgaagagttattaaagaatggcgcagatatgaacgcacaggataaattcggcaagacaacattcgacatctcaacagacaacggcaatgaagaccttgctgagattcttcaaaaactgggaggtggtggttccggtggaggcggttcccaagtgaagttagaagagtccggaggaggatcagtccaaacgggcggtagtcttaggctgacctgcgcagcatccggacgtacatctcgttcttatggaatgggctggttcagacaagctcccggtaaagaaagagagtttgtttctggtatttcttggagaggtgattcaacaggctatgctgactctgtaaaaggcagattcactatctcacgtgataatgcaaagaacacagtggatcttcaaatgaactccctaaaaccagaagatacggctatttattactgtgcagccgccgctggctccgcttggtatggaacgctttacgaatacgattattggggtcagggtactcaggtgaccgtttccagtggaggaggaggatccacattacctgtttacaccttgaaggagcgttgcttgcaagtggttagatcattagtgaaacctgaaaattacagaaggctagatattgttagatccctttatgaggacctagaggatcatccaaatgtgcagaaagacttagaaagactgacgcaagagaggattgcccaccagaggatgggagac

SEQ ID NO: 361 - DNA, 1014 bp, Artificial Sequence
  Description of Artificial Sequence Synthetic polynucleotide
tgcgatctgggtaaaaaacttttagaggcagccagggcaggccaagacgatgaggttagaatcttgatggctaatggtgccgacgtgaacgcatccgatagatggggttggacaccattacaccttgcagcttggtggggccatctagagatcgtagaggtgttgctaaaacgtggtgccgatgtatcagcagctgatctgcatggtcagtctcctctgcaccttgctgctatggtgggacatttggagatcgtggaggtgctattgaaatacggtgccgacgttaatgcaaaggatacaatgggagctacgcccctgcatctagcagctcgttcaggtcacttagaaatagttgaagaattactgaagaacggagctgatatgaatgcacaagataagtttggtaaaacgacgttcgatataagtacggataacggcaacgaggatctggcagagattctacaaaaattgggcggaggcggatctggtggtggcggatcacaggtgaagttagaggaatctggaggaggctctgttcagacgggcggctctctgcgtttgacttgcgctgcatctggtagaacatcaaggtcatacggcatgggttggttccgtcaggccccaggaaaagagagggagttcgtgagtggtatatcctggcgtggagattccacaggttatgcagatagtgtaaagggtcgtttcacgataagtcgtgacaacgcaaaaaacaccgtggacttacagatgaacagtttgaaaccagaagataccgctatttactattgcgcagcagccgcaggttccgcatggtacggaacattatatgagtatgattattggggacaaggcacccaggttaccgtttctagtggaggaggaggatcattacccgtgtatacgcttaaagagaggtgtcttcaagtggtacgttctttagtcaaacccgaaaattacaggcgtcttgatattgtacgtagtctatacgaggatctagaagatcatccaaacgtg

SEQ ID NO: 362 - DNA, 1011 bp, Artificial Sequence
  Description of Artificial Sequence Synthetic polynucleotide
tgcgatttgggtaagaaactgctagaggctgccagagccggacaagatgatgaggtcagaatacttatggctaatggcgccgacgtgaatgcctcagaccgttggggttggacaccattgcaccttgccgcctggtggggtcatttggaaattgtagaggtcctattgaagaggggagctgacgtttccgcagccgacttacacggccagagtcctttacatcttgctgctatggtcggccacctagagatcgtagaggttcttctgaagtacggagctgacgtgaatgctaaagatactatgggtgccacccctctgcaccttgcagcaagatcaggacacctggagatcgtagaggagctactaaagaacggagctgatatgaatgcacaggataagtttggcaagaccaccttcgatatttcaaccgataacggaaatgaggatttggctgaaatacttcaaaagctaggtggcggtggttccggcggtggcggctctcaagtaaagttggaagaaagtggaggtggatctgttcaaactggcggctcccttaggcttacttgtgccgcctccggaaggacgtcaaggtcctacggcatgggctggtttagacaagctccaggcaaagagagggagttcgtatccggaatctcatggagaggtgattccacaggatacgctgacagtgtgaagggcagattcacaattagtcgtgataatgccaagaacacagtggatctacagatgaactccctgaagcctgaagataccgccatctattattgtgcagccgccgctggctctgcctggtacggcaccttatatgagtacgactactggggacagggaacccaagtgaccgtgtcttctggcggaggtggaagtgttagacctttgcaggagctgtgtagacagcgtattgttgcaacagtcggacgtgaaaacttagcacgtattccactgaaccccgtccttagagactatctttcatcatttcccttccaaata

SEQ ID NO: 363 - DNA, 1047 bp, Artificial Sequence
  Description of Artificial Sequence Synthetic polynucleotide
gatctgggtaaaaagctactagaggctgcaagagctggccaagacgacgaagttcgtatcctgatggctaatggcgcagatgtgaacgcatccgatcgttggggctggacacctctacaccttgccgcatggtggggccacttggaaatcgttgaggttttacttaaacgtggtgcagacgtgtccgccgctgacctacacggtcaatctccactgcatctggctgccatggtaggacacctagaaatcgtggaggtattgttgaagtacggcgccgatgtcaacgctaaagacacaatgggtgcaacgccattacatcttgccgctagatcaggacacttggagatcgtagaggagctgctaaagaatggcgctgacatgaatgcacaggataagtttggtaaaactacttttgatatttccacagacaacggcaacgaggatctggccgaaattttgcagaagctaggcggcggcggatctgagcccaaatcatccgataaaacacacacctgtcctccatgtcctggacagccaagggagccacaagtctacaccttacctccttcaagagaagaaatgactaaaaatcaagtctccttgacttgcttagtaaagggattctacccatctgacatcgctgtggagtgggagtccaacggccagccagagaacaactataagactacgcctccagtgctagacagtgatggctccttttttctgtactccaagttaacggttgataagtctagatggcaacagggaaatgtattcagttgttccgtgatgcatgaagctctgcacaatcactatacccaaaagtctctttcattaagtccaggaggcggcggaggatctacattaccagtttacacgttaaaagagaggtgtttgcaagttgttcgttcccttgtcaaacccgagaactatagacgtttggacatcgtcaggtctttgtatgaggatctggaggatcaccccaacgtgcaaaaggacctagagagattgacacaagaacgtatcgcacatcaaaggatgggcgac

SEQ ID NO: 364 - DNA, 1050 bp, Artificial Sequence
  Description of Artificial Sequence Synthetic polynucleotide
accttgcccgtctatacccttaaggaaaggtgcctgcaggtagttagatccctagtaaagcccgaaaactacagaaggttggacatcgtcagatcactgtatgaagaccttgaggaccatccaaacgtgcaaaaagacctagaaagactaacacaagagaggatcgctcaccagagaatgggagacggaggcggaggtagtgatctgggaaaaaagttactagaagccgccagagctggacaagatgatgaggtaaggatactaatggcaaatggcgcagacgtgaacgcatcagataggtggggctggacaccattgcatctggccgcatggtggggacacctagaaatcgtagaagtgttgttgaaaaggggcgctgatgtgtctgcagccgacctgcatggacaatcacccctgcacctagctgctatggttggccatttagagatcgttgaagtgctactgaaatatggagcagatgtgaacgccaaagatacgatgggtgctacacctcttcatttagcagcaaggtcaggacacctggaaatagtagaggagttactgaaaaatggtgctgacatgaacgcccaggacaaatttggaaagaccactttcgatatatctactgacaacggtaatgaggatcttgccgagatactgcagaagcttggaggaggaggctccgaaccaaagtctagtgacaagacgcacacgtgtcctccttgtcccggccaaccaagagagcctcaggtttacacgttgcctccatccagggaggaaatgaccaagaatcaggtatccttaacatgtctggtgaagggtttttatccttcagatattgccgtggagtgggaatctaacggccaaccagaaaacaactataaaactactccaccagtgctggactccgacggctcattcttcttgtattcaaaactgaccgtagacaaatcccgttggcagcagggtaatgttttctcctgttcagtaatgcacgaagccctgcacaaccactatacacaaaaatcactttccctttctcccggttgc

SEQ ID NO: 365 - DNA, 1314 bp, Artificial Sequence
  Description of Artificial Sequence Synthetic polynucleotide
tgcgacttgggtaagaaattgctagaggcagctagagccggtcaggacgacgaggttcgtatcttgatggctaacggagctgacgtaaatgcctccgaccgttggggttggacgcccctacatctggccgcatggtggggccacctggaaattgtagaggttttacttaagaggggcgcagacgtgagtgctgctgaccttcacggacagtctcccctacatttagcagcaatggtcggtcaccttgagatcgtcgaggtgcttttaaagtatggcgcagacgtcaatgcaaaggacactatgggcgccactcctcttcatcttgcagccaggtctggacacctagaaatagtcgaggagctactaaaaaatggagctgacatgaatgcccaagacaagtttggtaaaactacatttgacatctctacagacaatggaaacgaggatttggctgaaatactacaaaaacttggaggaggtggctctgcctccgccgctaggcttacaatgatgtgggaagaggttacgtgtcccatctgcctggaccccttcgtcgagcccgtcagtatcgaatgcggtcactctttttgtcaagaatgtataagtcaagttggtaagggaggtggctccgtgtgcccagtgtgcaggcaaaggtttctgttaaaaaatttgcgtcctaatagacaacttgccaacatggtaaacaatcttaaagagatctcccaggaagccagggagggaacacaaggagaacgttgtgccgttcatggcgaacgtcttcatttgttctgtgagaaagatggtaaggcattatgctgggtgtgtgcccaatcccgtaagcaccgtgaccatgccatggtacccctagaagaggctgcccaagaataccaagagaaattgcaagtggctcttggtgagcttcgtagaaagcaggagctagccgagaaattagaagtagagatcgcaattaagagggctgattggaagaagaccgtcgagactcagaagtctcgtattcacgccgagttcgtccagcaaaagaactttctggtagaggaggagcagcgtcaattacaagagcttgagaaggacgaacgtgagcagcttcgtatcctgggagaaaaggaagctaaacttgcacaacaatcccaggcccttcaagagctgattagtgagttggatcgtaggtgtcactcctctgccttagaactattgcaagaagtaatcatcgtcctagagcgttcagaatcttggaacctgaaggacttggatataacatcaccagagcttcgttcagtctgtcatgtacctgga

SEQ ID NO: 366 - DNA, 1317 bp, Artificial Sequence
  Description of Artificial Sequence Synthetic polynucleotide
atggctagtgcagcccgtttaacaatgatgtgggaggaagtcacatgcccaatatgtcttgaccctttcgtcgagcctgtatctatcgaatgtggccattctttctgccaagagtgtatctctcaggttggcaagggcggaggatccgtttgccccgtttgcaggcagcgttttttgctaaaaaacttgaggccaaaccgtcaactggctaacatggtgaataatttaaaggaaattagtcaggaggcaagagagggaacacaaggagagagatgcgccgtgcacggcgaaagactgcatcttttttgcgaaaaagatggtaaggccctttgttgggtctgtgcacaatccaggaagcaccgtgatcacgcaatggtccccctagaagaggcagctcaagagtatcaggagaaattacaggtcgctttgggcgaactgcgtcgtaaacaggaacttgcagagaaacttgaagtagaaatcgctattaagcgtgccgattggaagaaaactgtcgagacacagaagtctagaattcatgctgagttcgttcaacaaaagaactttttggttgaagaggaacagcgtcagctacaagaattagagaaggacgagagagagcagcttcgtatccttggtgagaaggaggccaagctggctcaacagtcccaagccctacaagaactaatttccgaactggacagaagatgccactcatctgcattagagctacttcaggaagtcattattgtactggaaagaagtgaatcctggaacctgaaggatttggatattaccagtcctgaactaagaagtgtgtgccacgtaccaggaggaggcggaggctccgacttaggaaagaaactactggaggccgctagggctggacaagatgacgaagtaaggattttgatggcaaacggagccgacgttaatgccagtgatagatggggatggacaccattgcacctggctgcatggtggggacatctggagattgtcgaggtactgttgaaacgtggagccgatgtatcagcagctgatctacatggacagtctcccttgcacttggccgcaatggttggacatcttgaaattgtggaagttttgttaaaatatggcgctgacgttaatgcaaaggacaccatgggagccacgcccttacatctagcagccagatccggtcatttagagatagtcgaagaattactgaaaaatggtgccgacatgaatgcccaagacaaattcggaaaaacgacctttgatatatctaccgataacggtaacgaagatttagcagaaatacttcagaagctgtgc

SEQ ID NO: 367 - DNA, 1716 bp, Artificial Sequence
  Description of Artificial Sequence Synthetic polynucleotide
tgtcaagtcaaactagaagaaagtggaggtggctccgtccagactggtggatcattgaggcttacatgcgctgcttctggcagaacatcaaggtcatatggtatgggttggtttcgtcaggcacctggcaaagagagggagttcgtctctggaattagttggcgtggagattctaccggctacgctgattcagtcaagggacgtttcactatatctagagacaatgcaaaaaataccgtggatcttcagatgaactcactgaagcctgaagacaccgctatctactattgtgctgccgccgctggttcagcctggtacggcaccctatatgaatacgactattggggccagggcacccaggtcactgtatcctctggaggtggcggttctggaggcggtggctccgatctgggtaagaagctgcttgaagcagcacgtgccggtcaagacgatgaagtaaggattttgatggctaacggcgctgatgttaatgcatcagataggtggggctggacccctttgcacctagcagcttggtggggccaccttgagatagtcgaagttctattgaagcgtggcgccgacgtgtccgccgcagatttacacggccaatcacccctgcacttggctgccatggtgggacacttagagatcgttgaggtgctactgaagtatggagccgatgttaacgcaaaggatactatgggcgctactcccctacacttagctgcacgtagtggccatttagagatagtggaagaactattaaagaacggagcagacatgaacgcacaagataagttcggtaagaccacattcgatatttctacggacaatggcaatgaagacctggccgaaatactacaaaagctgggaggcggtggatctgcatccgccgctaggttgactatgatgtgggaagaagttacatgtccaatctgtctagatcctttcgttgaacctgtctctattgaatgcggccactcattttgtcaagagtgtattagtcaggtgggtaagggcggtggctcagtgtgtccagtctgtaggcagagatttttgctgaagaatttgaggcctaaccgtcagttggccaatatggttaacaacctgaaagagatatcccaagaggccagagagggcactcagggcgagagatgtgccgtacacggtgagaggctgcaccttttctgcgaaaaagacggcaaggctttatgctgggtctgcgcccagtccaggaaacacagagaccatgccatggtgccattagaagaagcagctcaggagtatcaggagaagctgcaagtagcacttggagagcttaggcgtaagcaggagctagcagagaaacttgaggttgagatagccatcaagagggctgattggaaaaaaacagttgagactcagaagtcccgtatacacgctgagttcgtacaacaaaagaactttttggtcgaagaggagcagcgtcaactgcaggaacttgagaaggacgagagggagcagttacgtattctaggcgaaaaggaagcaaaactggcccaacaatcccaagctcttcaagagcttattagtgagctagacagacgttgtcactcttcagcccttgagctgctacaggaagttatcatcgtacttgaaaggagtgaatcctggaatttgaaggacttggatatcacatctccagaattgaggtctgtctgtcatgtgcctgga

SEQ ID NO: 368 - DNA, 1059 bp, Artificial Sequence
  Description of Artificial Sequence Synthetic polynucleotide
caagtacaattggttgagtctggcggaggattagtccagcctggtggatctcttacactttcttgtaccgcttccggcttcacactagatcactacgacataggttggttcagacaggctcccggcaaagagagagaaggtgtctcctgtataaataactctgacgatgacacgtattacgctgatagtgtcaagggtaggttcaccattttcatgaataacgcaaaggacacggtttacctacaaatgaattctctgaaacctgaagacacagccatttactactgtgctgaagccagaggatgtaaacgtggtcgttatgagtacgacttctggggccagggaacacaagttactgtctcctccggcggaggaggtagtgataagacccatacttgtcccccttgccctgcacctgagctgttgggcggaccatgcgtgttcttgtttccacccaagccaaaagataccttaatgatttcacgtacccccgaggtcacctgcgttgtggttgatgtgtcacacgaagatccagaggtgaagtttaactggtacgttgatggtgttgaagtccataacgctaaaaccaaaccaagggaggaacagtacgcctctacataccgtgtcgtgtcagttctgaccgtgttacaccaagattggcttaacggcaaagaatacaagtgtaaagtctcaaacaaggctttacccgctccaatagagaaaaccatatcaaaagctaagggccagcctcgtgaacctcaggtttacactcttccaccatccagggaagaaatgaccaaaaatcaagtttctctaacctgcttagttaagggtttttacccttcagacatagcagtcgagtgggagtctaatggccagcccgaaaacaactataaaactactcctcccgtgcttgatagtgacggttctttcttcttatattccaaattgactgtcgataaatccaggtggcaacaaggtaacgtcttttcatgctccgtaatgcatgaggctcttcataatcactacacgcaaaaatccctatcccttagtccagga

SEQ ID NO: 369 - DNA, 948 bp, Artificial Sequence
  Description of Artificial Sequence Synthetic polynucleotide
tgtcaggtacaacttgtagaatccggcggcggtcttgtccagccaggcggctccctgacgctttcctgtaccgcaagtggctttactctagaccattatgacattggctggttcaggcaggcccctggcaaggagagagagggcgtatcctgtattaataactccgacgacgacacctattatgcagatagtgtaaaaggcaggtttaccatcttcatgaacaacgcaaaagacacagtatacctacaaatgaatagtctgaagccagaggatacagccatttattattgtgcagaagcaagaggctgcaaaaggggacgttatgagtacgatttctggggacaaggcacccaagttacggtatcatcagagccaaaatcctcagataaaacacacacatgccccccttgtcccggccaaccaagagagccccaggtgtatacgctgcccccaagtagggaagaaatgactaaaaaccaagtttctttaacgtgcttagttaagggattttacccttcagacatagccgttgagtgggagtctaacggccaacctgagaacaactataaaactacccctcccgtcttagattcagatggttcattttttctgtactcaaagctaacggtcgataagtcaaggtggcagcaaggcaacgtatttagttgctctgtaatgcatgaggcccttcacaaccattatactcagaaaagtctatctctttctcctggtggtggcggcggctcaggcggcggtggttccactcttcccgtctataccttaaaggagcgttgcctacaggtagtgagaagtttagttaagcctgagaattacaggagattagacattgtgcgttcattatacgaggacctagaggatcatcccaacgtgcaaaaggatctggagagacttacgcaggagaggatagctcatcaacgtatgggtgac

SEQ ID NO: 370 - DNA, 948 bp, Artificial Sequence
  Description of Artificial Sequence Synthetic polynucleotide
acactaccagtttatacgctgaaagagagatgcctgcaagtagtccgttctttggtcaaaccagagaactatagacgtctagacatcgtaaggtctctttatgaggatcttgaagatcatcccaacgttcagaaggacttggagagattaacccaggaacgtatcgcacaccagagaatgggcgacggtggcggtggctccggtggtggtggctcacaggttcagctagtagaatcaggtggaggtctggttcaacccggcggttccctaactttgtcctgcaccgcatctggattcacccttgaccactatgacataggctggtttcgtcaggcccccggcaaagagagagagggagttagttgtataaacaactcagatgatgacacttattacgcagattcagtgaagggccgttttactatcttcatgaataatgctaaagataccgtatacctacaaatgaactctttgaaacccgaggacacggccatatactattgcgccgaagcccgtggttgcaagcgtggcagatacgaatatgacttttggggtcagggaacccaggtcacagttagttcagagcctaaatctagtgataagacccatacgtgcccaccctgcccaggccaacccagggaacctcaggtgtatactttgcccccaagtagagaggaaatgacaaagaatcaggtgtcactaacctgtcttgttaagggcttctatccatctgacatagccgtcgaatgggagagtaatggacaacccgagaataactataaaactacgcctcccgtattggactctgatggatcattctttttatacagtaaattaacggtagataagtccaggtggcagcagggaaacgtcttctcttgttctgtgatgcacgaagcactgcacaaccattacactcaaaaatctttgagtttgtcaccaggctgc

SEQ ID NO: 371 - DNA, 738 bp, Artificial Sequence
  Description of Artificial Sequence Synthetic polynucleotide
tgcactctaccagtctacacacttaaagaaagatgtttacaggttgtgcgttcactggtgaagccagaaaattaccgtcgtttagacatcgtgaggtccttatacgaagatctggaagatcatcctaacgtacaaaaagatcttgagcgtcttactcaggagcgtatagcacatcagaggatgggagacggaggaggaggcagtggtggaggtggctcacaagtgcagctagtggaatccggtggcggattagttcagccaggcggctcactgactctatcttgtacggcaagtggtttcacgttagatcattacgacatcggttggttcaggcaagctcctggcaaggagcgtgaaggcgtctcctgtatcaataacagtgacgatgacacgtactatgccgattcagtgaagggtcgttttacgatcttcatgaacaatgccaaggatacggtctacttgcaaatgaacagtttgaagcccgaggataccgctatatattattgcgctgaggcccgtggctgcaaaaggggtaggtatgaatacgatttctggggacagggaactcaggtcacggttagtagtggaggaggtggcagtggcggaggcggttcagtgagaccccttcaggagttgtgccgtcagcgtatcgtcgcaacagttggaagagaaaatctagctcgtataccattgaatccagttctaagggattacttgtcttccttcccctttcagatt

SEQ ID NO: 372 - DNA, 585 bp, Artificial Sequence
  Description of Artificial Sequence Synthetic polynucleotide
acgttgccagtatatactcttaaagagaggtgcttacaggtcgttcgttctctagttaaacccgagaattataggagactggacatcgtgcgtagtctgttcgaggatctggaggaccatcccaacgtccaaaaggatctagaaagactaacgcaagaaagaattgctcatcaaaggatgggagatggcggtggtggctcaggtggaggcggatcacaagttcagcttgtcgagtcaggcggtggactggtacagcctggaggatccctgacgttatcatgcacagcctccggttttaccttagaccactacgatattggctggtttaggcaggcacccggtaaagagagggaaggtgtgtcctgtataaataattctgacgatgatacatactacgccgattcagtgaaaggtcgtttcacaatttttatgaacaacgccaaagatacggtgtacctacaaatgaacagtctaaaaccagaagacacggccatctactactgtgccgaagctcgtggctgtaaaagaggacgttatgagtatgatttctggggtcaaggtactcaagttacggtatcatcctgt

SEQ ID NO: 373 - DNA, 1186 bp, Artificial Sequence
  Description of Artificial Sequence Synthetic polynucleotide
tgtcaggttcaactggtggagtctggcggcggcttggttcaacccggaggctctttgaccttgtcttgcaccgcttccggttttacccttgaccattatgatataggatggttcaggcaagcccccggtaaagagcgtgagggagtatcctgcatcaacaattccgacgacgacacgtactatgccgactccgtgaagggtaggtttaccatttttatgaacaatgctaaggatacggtatatctgcaaatgaattccttgaagcccgaggacaccgccatctattactgtgcagaagctagaggctgtaaaagaggaagatacgagtacgacttctggggtcagggcactcaggtaaccgtctcctctggaggaggaggcagtggaggaggtggatctgcatcagcagcccgtctgactatgatgtgggaggaagtcacatgtcccatatgtttagaccccttcgtagagcctgtctcaatcgaatgcggacattccttttgtcaagagtgtatctctcaggttggtaagggaggtggctctgtctgccccgtctgccgtcaaaggttcctattgaagaacctgaggccaaatcgtcagttggctaacatggtgaataaccttaaagagatctcccaagaagccagagaaggcacgcaaggcgaacgttgcgcagttcatggcgagagactacatctattctgcgagaaggacggtaaagcattgtgctgggtctgtgcacagagtaggaagcatagggatcacgcaatggtgccacttgaagaagccgcccaggaatatcaggaaaaactgcaggtagctttaggcgaattgagacgtaagcaagaattggcagagaagctggaggtagagatagcaataaagcgtgccgattggaaaaaaactgtggaaacgcagaaaagtagaatacacgcagaattcgttcaacaaaaaaatttcctagtagaggaggagcagaggcaactgcaggagttggaaaaggacgaacgtgagcaactaagaatacttggcgaaaaggaagctaagttggcccagcaaagtcaagctttacaagagttgatttccgaattagatcgtcgttgtcactcttcagctcttgagctattgcaagaagttatcatcgtgctggagagatcagagtcatggaatctaaaagatttggaca

SEQ ID NO: 374 - DNA, 1263 bp, Artificial Sequence
  Description of Artificial Sequence Synthetic polynucleotide
catcatcaccaccatcatggagatgatgatgataaggcatcagcagcacgtctaacaatgatgtgggaggaagtcacttgtccaatatgtcttgatcccttcgtagagcctgtttcaatagagtgtggacattccttttgtcaagaatgcattagtcaggtaggtaaaggaggtggttctgtttgtcctgtatgcagacaaagattcttattaaagaacctacgtcccaacagacagttagcaaatatggtcaacaatctgaaagagatttctcaagaggcccgtgaaggaacacagggtgagagatgcgctgtccacggagagcgtcttcatttattttgcgagaaggatggtaaagcactttgctgggtatgtgctcaatcacgtaaacatcgtgaccacgccatggttcccctagaagaagctgcccaagagtatcaggagaaactacaagtggcacttggcgaactgaggaggaagcaagagttggccgaaaaacttgaggtcgaaattgccattaagagggccgattggaagaagactgtcgagacacagaaaagtagaattcatgcagaatttgtgcagcaaaaaaacttcttggttgaagaggagcagcgtcaattacaggaattggaaaaagatgaaagggaacagcttcgtatattaggagaaaaggaggctaaacttgcccaacagtcacaggctctgcaggagctaatctccgaacttgacagacgttgccactccagtgctttagagcttttgcaggaggtcatcatagtcctggagagaagtgaatcatggaaccttaaggatctggacattacctcacccgaactaagatccgtctgccatgttccaggtggcggtggcggatcaggtggtggcggatcacaggtacagctagtggagagtggtggaggcttagtgcaaccaggtggatcactaactttatcatgcactgccagtggcttcacactggaccactacgacattggttggtttagacaggcccccggaaaagaaagggaaggcgtatcctgtatcaacaattccgacgacgacacctattatgctgattccgtaaaaggtaggtttacgatttttatgaacaatgccaaggatacggtgtatctacagatgaattcccttaaacctgaagataccgcaatttattactgtgcagaggccaggggttgcaaaagaggcaggtacgaatatgacttctggggacaaggcacccaagtaacagtctcctcctgc

SEQ ID NO: 375 - DNA, 1443 bp, Artificial Sequence
  Description of Artificial Sequence Synthetic polynucleotide
accctacccgtgtacaccttaaaggagaggtgtctacaggtggttagaagtctagtgaaaccagagaactatagaaggcttgatatcgtgcgttccttgtacgaagacttggaggaccacccaaatgttcagaaggacttggaaaggttgacccaagagcgtatcgcacaccaacgtatgggcgatggaggcggtggcagtggaggaggaggctctcaagtacagttagtggaatcaggtggcggtttggtccaaccaggcggaagtcttactttgtcctgcacggcatccggtttcactttggaccactatgatatcggctggttccgtcaagcccctggaaaagaaagagagggcgtatcttgtatcaataacagtgacgacgatacatattacgccgactctgtaaagggaagattcaccattttcatgaacaacgccaaagacaccgtataccttcagatgaattccttgaagccagaggatacagctatctattattgcgctgaggctcgtggctgcaaaaggggaagatatgagtacgacttctggggccaaggcacgcaagttacagtctccagtggtggcggaggctctggtggaggaggatctgcttctgcagcaaggttgaccatgatgtgggaggaggtgacgtgtcccatttgtcttgatccattcgtcgagcccgtgtccatagaatgtggacactctttctgccaggagtgtatttcccaggtcggcaaaggtggtggatcagtgtgtccagtctgccgtcagagattcctattgaagaatctgagacccaacagacagctagctaacatggttaataacttaaaagaaatcagtcaagaagcaagagagggtacgcagggagagcgttgcgcagttcacggagagagactacacctattctgcgaaaaggacggcaaggcattgtgttgggtgtgtgcacaaagtcgtaagcacagagatcatgctatggtgcctttggaagaggccgcccaagagtatcaggagaagttgcaagtcgccttgggagagttaagaaggaagcaggagttagctgaaaaactagaggtcgagatcgccataaagagagcagactggaaaaagacggtcgagacacaaaagtcaaggatacacgccgaattcgtgcagcagaagaacttcctagtagaagaagagcaaaggcaattgcaagagctagaaaaggatgaaagagaacagctaaggattctaggcgagaaggaagccaagctggctcaacaatctcaagccctacaagagttgattagtgagctagaccgtagatgccattcttccgcccttgaactactacaagaagtcatcattgttttggaaaggtctgagtcttggaacctgaaggatcttgacataaccagtcccgaactacgttctgtttgtcacgtacccggttgt

SEQ ID NO: 376 - DNA, 903 bp, Artificial Sequence
  Description of Artificial Sequence Synthetic polynucleotide
accctgcctgtttacacgctaaaggagagatgcctgcaagttgtcagatcattagtaaagccagagaattatagaaggctggatattgtaaggtcactatatgaggacttagaggatcatcccaatgtccaaaaggatcttgaaaggctaacacaggagcgtatagcacaccagaggatgggagatggcggaggtggcagtcagcttcagctagtagaaagtggaggtggtcttgtgcagcctggtggatccttaaagctgtcctgcgccgcatcaggcttcacattctctaggtatgccatgagttggtacaggcaggcccccggtaaagagcgtgaaagtgtagctaggatctcctccggtggaggaacgatatactatgccgattctgtaaagggcaggtttacaatctcccgtgaagacgcaaagaacacagtctatttgcagatgaattcacttaagcctgaagatacggctgtatattattgctacgtgggcggcttttggggtcagggaactcaggtgaccgtctcctctgagcctaaaagtagtgataagacgcatacctgtcctccttgtcctggacagccaagagagccacaggtctatacactgccaccttcaagggaagagatgacgaagaaccaggtaagtctgacctgccttgtgaagggcttttacccttctgatattgctgtggaatgggaatccaacggtcaacccgaaaacaactataagacaacaccacccgtacttgattccgatggtagttttttcttgtatagtaagctgactgtggataaatccagatggcaacagggaaacgtattctcctgctccgtaatgcatgaggctctgcacaaccattatactcagaaatctttaagtctatcaccaggctgt

SEQ ID NO: 377 - DNA, 480 bp, Artificial Sequence
  Description of Artificial Sequence Synthetic polynucleotide
tgtcagttgcagttggtagaatccggtggcggtctagttcaaccaggcggatcattgaaactttcatgtgctgcatccggatttacattctctcgttatgctatgtcttggtatagacaggcacctggaaaggagagggaatccgttgctcgtattagttccggtggaggtacgatctattacgccgactcagtaaaaggtaggtttaccataagtagagaagatgcaaagaacacagtttacttgcaaatgaacagtttaaagcccgaagacactgcagtgtactactgctatgtcggcggcttttggggccaaggaacgcaggtcaccgttagttctggaggaggtggttctctgccagtctatactctaaaggagagatgcttgcaggtagtcaggtcattagttaaaccagaaaactacaggagacttgatatagtacgttctttatacgaggatttagaagaccatcctaacgtg

SEQ ID NO: 378 - DNA, 540 bp, Artificial Sequence
  Description of Artificial Sequence Synthetic polynucleotide
acgctacccgtctacacgttgaaagagaggtgtcttcaggtcgttcgttctttagtaaagcccgaaaactacaggaggctggacatcgtaagatccctgtatgaggatctggaagaccaccccaatgtacaaaaagacttagagagattaacgcaggagagaatcgcccaccagcgtatgggtgacggtggtggcggaagtcaacttcagctagttgaatctggtggcggactggtccagcctggcggttctctaaagcttagttgtgccgctagtggatttacattttcaaggtatgctatgtcctggtacagacaggctcccggcaaggagagagaatccgttgctagaatatcttccggtggtggcactatctactacgctgattctgtcaaaggtcgtttcactatatctagggaagatgccaagaataccgtctatttgcaaatgaatagtctgaagcccgaagacactgccgtctactactgttatgttggaggcttctggggtcaaggtacgcaggttacagttagttcatgt

SEQ ID NO: 379 - DNA, 678 bp, Artificial Sequence
  Description of Artificial Sequence Synthetic polynucleotide
tgtaccctgcctgtgtacactctaaaagaaaggtgtcttcaggtcgttagatcccttgtaaagccagaaaattataggagattagatatcgtgcgttcactttacgaggacctggaggaccatccaaacgttcagaaggacctagagagactgacccaagagaggattgctcaccagagaatgggcgacggtggaggaggatctcaactgcaattagtggagtcaggtggcggcttagtgcaaccaggaggcagtttgaagctaagttgtgccgcatccggctttaccttttctcgttacgcaatgtcatggtatcgtcaggctccaggaaaggaacgtgagagtgtagcccgtatctcatctggaggtggcaccatctactatgccgattcagtgaagggtaggttcacaatttctcgtgaagacgcaaagaacacggtgtacctacaaatgaatagtttgaagccagaagacacggccgtctattattgctatgtcggtggtttttggggtcaaggaacgcaagttacagtatcatccggcggcggcggctctgtaaggccattgcaggaactatgtaggcaacgtattgttgccacggtgggtagagagaacctggccaggatacccttaaatccagtgcttcgtgactatctgtcttcctttccatttcagatc

SEQ ID NO: 380 - DNA, 678 bp, Artificial Sequence
  Description of Artificial Sequence Synthetic polynucleotide
gttcgtcctcttcaagaactatgccgtcagagaatagtagctactgtgggtagggagaaccttgctagaattcctctgaatccagttttaagagactatttgtcctcctttccattccagatcggaggcggcggaagtactcttcccgtgtacacgctgaaggaaaggtgtttacaggtcgtgaggtcattggtgaagcccgagaattatagaagactagacatagtacgttccctgtatgaggatttagaggatcatccaaatgttcagaaggatttagaaagactgacgcaggaaagaatagcccaccaaagaatgggtgacggtggtggaggctcacaactacaacttgtagagtccggtggtggattagtccagcccggaggatctctaaaactatcctgcgctgcatctggtttcactttttcaaggtacgccatgtcttggtatagacaggcaccaggtaaagagcgtgagtccgtcgccagaatttcctcaggcggtggtacgatctactatgctgattctgtgaaaggtaggtttacaatatcaagggaggatgcaaagaacactgtctacctacagatgaattcactgaaacctgaagacacagccgtttactattgttatgttggaggtttttggggacagggaactcaagtgaccgtatcttcatgt

SEQ ID NO: 381 - DNA, 1182 bp, Artificial Sequence
  Description of Artificial Sequence Synthetic polynucleotide
caattgcaattggtggaatctggcggtggcttagtacagcctggtggatctttgaagctatcttgcgccgcttctggctttacgttttcccgttatgcaatgtcatggtacaggcaggcccccggcaaagaaagggaaagtgtggccagaatctctagtggtggcggaacaatatattatgcagattccgtaaaaggaagatttacgatttctcgtgaggatgctaagaatacagtgtatttgcagatgaactcattaaagcctgaagatactgccgtgtattattgctacgtaggaggtttttggggccaaggtactcaggtgactgtctcctctggtggtggaggctcagcttctgccgcccgtctaacaatgatgtgggaggaagtgacgtgtccaatctgcttggacccatttgtggagccagtctcaatagaatgtggacattcattctgtcaggaatgcatcagtcaagttggaaagggcggaggatcagtctgccccgtctgtagacaaagattcctgttaaagaacctaaggccaaaccgtcagctggcaaacatggtaaacaacctaaaggagatttcacaggaagctcgtgaaggaactcagggcgaaagatgcgccgtccatggagaaagattacacctgttctgcgagaaggacggcaaggctttgtgttgggtctgtgcccaatcccgtaaacacagagaccacgctatggtccctctggaggaggccgcacaagaataccaagaaaagttacaagtggcactgggagaactgcgtagaaaacaagaactagcagagaaattggaggtcgaaatagctattaaaagagccgactggaagaagaccgtggaaactcaaaagtctaggatccatgcagagtttgtacaacagaaaaacttcttagtagaagaggagcagagacaattacaagagcttgagaaggatgagagggagcagcttcgtatattaggagaaaaggaggcaaagttggcccagcaatctcaggctttacaggaattaatttcagagttagataggagatgtcattcctcagcactagagttacttcaggaggtaatcatagttttggaaaggtctgagtcatggaacttaaaggatcttgatattacgagtccagaattacgtagtgtttgccacgtacccggctgt

SEQ ID NO: 382 - DNA, 1185 bp, Artificial Sequence
  Description of Artificial Sequence Synthetic polynucleotide
atggcctccgcagccagattaacgatgatgtgggaagaggttacatgtcccatctgcttagatcctttcgtcgaacctgtctcaattgagtgcggacactcattttgccaagaatgtatctcacaagtcggaaaaggaggcggttctgtgtgcccagtttgtcgtcaaagatttctgttgaaaaatctgagaccaaacagacaactggctaacatggtcaacaatttaaaggaaatatcccaagaagctcgtgaaggaacccagggcgagcgttgtgccgtacatggcgaaagattacatttattctgtgaaaaggatggaaaagctttatgttgggtctgtgcacaatccagaaagcacagggatcatgcaatggtcccccttgaagaggcagcacaggaataccaggaaaagttgcaagtagcactgggagaacttaggcgtaagcaggagttggctgagaaactagaagtcgagattgctattaaacgtgccgactggaagaagacggtcgaaactcagaaatctaggatacatgctgagttcgttcaacaaaaaaattttctggtcgaagaggagcaaaggcagttgcaggaactggaaaaagatgaacgtgaacaattaagaatcttgggagagaaggaagcaaaacttgcccagcaatctcaagcacttcaggagctaattagtgaactagacaggaggtgccatagttccgcccttgaactattgcaagaagtaattatcgtactagagcgttccgagtcttggaatctaaaagacttggatatcacctcccccgaattacgttcagtttgtcacgttcccggtggtggaggaggctcccaattacagttagtcgaatctggcggcggattggtccagcctggaggatcactaaagctgagttgtgccgcatcaggatttacgttcagtagatacgctatgagttggtacagacaggctccaggaaaagagagggagtctgtagccaggatctcctctggtggaggaacgatttattatgctgattctgtaaagggaagatttacaatcagtcgtgaagatgccaagaacacagtatatttacaaatgaattccctgaagccagaagatacggccgtttattactgttatgtaggaggtttttggggtcagggtacgcaagtaacagtttcttcttgt

SEQ ID NO: 383 - DNA, 1383 bp, Artificial Sequence
  Description of Artificial Sequence Synthetic polynucleotide
actttgcccgtatatacgctaaaggaaaggtgtctacaggtcgtgaggtcacttgtgaagccagagaactatagacgtttggacatagtaaggtccctatacgaggatttggaagatcatcctaacgtccagaaagacctggaaaggctaacccaggaaagaatagcccatcagaggatgggcgatggaggtggcggctctcagttacagctggtggaatccggcggtggtttggtacaacccggcggcagtcttaagttatcatgtgccgcttccggtttcaccttcagtagatacgctatgagttggtacagacaggctcccggaaaagaaagggagtccgtagcacgtatcagttcaggaggaggtacgatctactacgcagactcagtcaagggcaggttcacgatttcaagggaggacgccaagaataccgtttacctgcagatgaactctttaaagcctgaagacactgccgtgtactactgctatgttggcggcttctggggtcagggcacgcaagtaactgtctcctccggaggtggcggttcagcctctgctgcccgtctgactatgatgtgggaagaagtgacctgccccatttgtttggacccttttgtcgaacctgtttcaattgagtgtggacactctttttgtcaagagtgtatcagtcaggtgggcaaaggcggaggatctgtatgccctgtgtgccgtcagagattcctattgaagaacttgcgtccaaatcgtcaattagcaaacatggtgaataacctaaaagaaatctcccaagaggccagagaaggcacgcaaggagaaagatgtgcagtccatggcgagagactacatttattttgtgaaaaggacggcaaagccctatgttgggtttgcgctcagtccaggaagcatcgtgatcacgcaatggttccattagaggaagctgcccaggaatatcaggaaaaactacaggttgctcttggcgaactgagaagaaagcaagaactggcagagaaattggaggtggaaattgctatcaaacgtgcagattggaaaaaaaccgtggagactcagaagtccaggatacatgcagaatttgttcagcagaagaattttctagtagaagaggaacagagacagcttcaagaactagagaaggatgaacgtgagcagttgagaattttaggagaaaaggaagcaaagttagcccaacagagtcaggcactacaagagcttatctcagagcttgaccgtagatgccattctagtgctctagagctactgcaggaggtgataatcgtcttagaaaggagtgaaagttggaacttgaaagacttggacatcacttcacccgagttacgtagtgtttgtcatgtccccggctgt

SEQ ID NO: 384 - DNA, 1164 bp, Artificial Sequence
  Description of Artificial Sequence Synthetic polynucleotide
gacctgggaaaaaagctgctggaagccgctagagccggccaggacgacgaggttagaatcctgatggccaacggggccgacgtgaacgccagcgacagatggggctggacccctctgcacctggctgcttggtggggacatctggaaatcgtggaagtgctgctgaagcggggcgctgatgtttccgccgccgatctccacggccagagccccctgcatctggccgctatggtgggccacctggaaatcgtggaagtcctgctgaagtacggcgcagatgtgaacgccaaagataccatgggcgctacacccctgcacctggccgccagaagcggccacctggaaattgtggaagagctgcttaaaaacggcgccgacatgaacgcccaggacaagttcggcaagacaaccttcgacatcagcaccgacaacggcaatgaggacctggccgagatcctccagaaactgggcggcggaggcagcgacaagacccacacctgccctccatgccccgcccctgagctgctgggcggaccttccgtgttcctgtttccccctaagcccaaggacaccctgatgatctctcggaccccagaggtgacctgtgtggtggtggacgtgtcccacgaggatcctgaggttaagttcaactggtatgtggacggcgtggaagtgcacaatgccaagaccaagcctcgggaagagcagtacgccagtacatacagagtggtcagcgtgctcaccgtgctgcaccaggattggctgaacggcaaagagtacaagtgcaaggtgtctaacaaggccctgccagcccctatcgagaagaccatcagcaaggccaagggccaacctagagagccccaggtgtacaccctgccacctagcagagaggaaatgaccaagaaccaggtcagcctgacctgtctggtgaagggcttctaccccagcgacatcgccgtggagtgggagtctaatggacaacctgagaacaactacaaaacaacacctcctgtgctggacagcgatggctcttttttcctgtacagcaaactgacagtggataagtcccgctggcagcagggcaacgtgttcagctgcagcgtgatgcacgaggccctgcacaatcactacacccagaagagcctgagcctgtctcctggctgc

SEQ ID NO: 385 - DNA, 1164 bp, Artificial Sequence
  Description of Artificial Sequence Synthetic polynucleotide
gacctgggaaaaaagctgctggaagccgctagagccggccaggacgacgaggttagaatcctgatggccaacggggccgacgtgaacgccagcgacagatggggctggacccctctgcacctggctgcttggtggggacatctggaaatcgtggaagtgctgctgaagcggggcgctgatgtttccgccgccgatctccacggccagagccccctgcatctggccgctatggtgggccacctggaaatcgtggaagtcctgctgaagtacggcgcagatgtgaacgccaaagataccatgggcgctacacccctgcacctggccgccagaagcggccacctggaaattgtggaagagctgcttaaaaacggcgccgacatgaacgcccaggacaagttcggcaagacaaccttcgacatcagcaccgacaacggcaatgaggacctggccgagatcctccagaaactgggcggcggaggcagcgacaagacccacacctgccctccatgccccgcccctgagctgctgggcggaccttgcgtgttcctgtttccccctaagcccaaggacaccctgatgatctctcggaccccagaggtgacctgtgtggtggtggacgtgtcccacgaggatcctgaggttaagttcaactggtatgtggacggcgtggaagtgcacaatgccaagaccaagcctcgggaagagcagtacgccagtacatacagagtggtcagcgtgctcaccgtgctgcaccaggattggctgaacggcaaagagtacaagtgcaaggtgtctaacaaggccctgccagcccctatcgagaagaccatcagcaaggccaagggccaacctagagagccccaggtgtacaccctgccacctagcagagaggaaatgaccaagaaccaggtcagcctgacctgtctggtgaagggcttctaccccagcgacatcgccgtggagtgggagtctaatggacaacctgagaacaactacaaaacaacacctcctgtgctggacagcgatggctcttttttcctgtacagcaaactgacagtggataagtcccgctggcagcagggcaacgtgttcagctgcagcgtgatgcacgaggccctgcacaatcactacacccagaagagcctgagcctgtctcctggcaag

SEQ ID NO: 386 - DNA, 1032 bp, Artificial Sequence
  Description of Artificial Sequence Synthetic polynucleotide
caactgcagctggttgaatctggcggcggactggttcaacctggcggatctctgaagctgagctgtgccgccagcggcttcacctttagcagatacgccatgagctggtacagacaggcccctggcaaagaaagggaaagcgtggccagaatcagcagcggcggaggcacaatctactacgccgatagcgtgaagggcagattcaccatcagcagagaggacgccaagaacaccgtgtacctgcagatgaacagcctgaagcctgaggacaccgccgtgtactactgttacgtcggcggcttttggggccagggcacacaagtgacagttagctctggtggcggaggctccgacaagacccatacctgtcctccatgtcctgctccagagctgctcggaggcccttccgtgtttctgttccctccaaagccaaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagccgggaagagatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 387 - DNA, 717 bp, Artificial Sequence
  Description of Artificial Sequence Synthetic polynucleotide
caactgcagctggttgaatctggcggcggactggttcaacctggcggatctctgaagctgagctgtgccgccagcggcttcacctttagcagatacgccatgagctggtacagacaggcccctggcaaagaaagggaaagcgtggccagaatcagcagcggcggaggcacaatctactacgccgatagcgtgaagggcagattcaccatcagcagagaggacgccaagaacaccgtgtacctgcagatgaacagcctgaagcctgaggacaccgccgtgtactactgttacgtcggcggcttttggggccagggcacacaagtgacagttagctctggtggcggaggctccgaacctaagtcttctgacaagacccatacctgtcctccatgtcctggccagcctagggaaccccaggtttacacactgcctccaagccgggaagagatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 388 - DNA, 1263 bp, Artificial Sequence
  Description of Artificial Sequence Synthetic polynucleotide
gattacaaggacgacgacgataagggcagcagcggccaccaccatcaccaccacgagaacctgtacttccaaggcatggaagccggcagacccagacctgtgctgagaagcgtgaacagcagagaacccagccaagtgatcttctgcaatcggagccccagagtggtgctgcccgtgtggcttaatttcgacggcgagccccagccttatcctacactgccacctggcaccggcagacggatccactcttatagaggacacctgtggctgttccgcgacgccggaacacatgatggcctgctggtcaatcagaccgagctgttcgtgcccagcctgaacgttgacggccagcctatcttcgccaacatcaccctgcctgtgtacaccctgaaagaacggtgcctgcaggttgtgcggagcctggtcaagcccgagaattacagacggctggacatcgtgcggtccctgtacgaggatctggaagatcaccccaacgtgcagaaggacctggaacggctgacccaagagagaatcgcccaccagagaatgggagatggcggcggaggcggagaatttgctatggctcaggtccagctgcaagagtctggcggaggacttgttcagcctggcggaagcctgaagctgtcttgtgccgccagcggcttcaccttcagcagatacgccatgagctggtacagacaggcccctggcaaagaaagggaaagcgtggccagaatcagctccggcggaggaacaatctactacgccgacagcgtgaagggcagattcaccatctccagagaggacgccaagaacaccgtgtacctgcagatgaactccctgaagcctgaggacaccgccgtgtactactgttacgtcggcggcttttggggccagggcacacaagtgacagtgtctagtggtggccagctccagctggtggaaagtggtggtggactggttcaaccaggcggctctctgaaactgagctgtgccgcctctggcttcacattctctcgctacgccatgtcttggtatcggcaggctcccggaaaagaacgcgagtctgtggccaggatctctagtggcggaggcaccatctattatgctgactccgtgaaaggccggtttaccatcagccgcgaggatgccaaaaacacagtctatctccaaatgaacagtctgaagcccgaagatacggccgtctactattgctatgttggcggattctggggacagggaacccaagtcactgtgtcctct

SEQ ID NO: 389 - DNA, 1422 bp, Artificial Sequence
  Description of Artificial Sequence Synthetic polynucleotide
gattacaaggacgacgacgataagggcagcagcggccaccaccatcaccaccacgagaacctgtacttccaaggcatgcccagacgggccgagaactgggatgaagctgaagtgggagctgaggaagccggcgtcgaggaatatggccctgaagaagatggcggcgaagagtctggcgccgaggaaagtggacctgaggaaagcggtcctgaagaactgggcgccgaagaggaaatggaagccggaaggcctagacctgtgctgcggagcgtgaacagcagagaacccagccaagtgatcttctgcaatcggagccccagagtggtgctgcccgtgtggcttaatttcgacggcgagccccagccttatcctacactgccacctggcaccggcagacggatccactcttatagaggacacctgtggctgttcagagatgccggcacacacgatggcctgctggtcaatcagaccgagctgttcgtgcccagcctgaacgttgacggccagcctatcttcgccaacatcaccctgcctgtgtacaccctgaaagaacggtgcctgcaggttgtgcggagcctggtcaagcccgagaattacagacggctggacatcgtgcggtccctgtacgaggatctggaagatcaccccaacgtgcagaaggacctggaacggctgacccaagagagaatcgcccaccagagaatgggagatggcggaggtggcggagagtttgctatggctcaggtgcagctccaagaaagcggcggaggacttgttcagcctggcggatctctgaagctgagctgtgccgccagcggcttcacctttagcagatacgccatgagctggtacagacaggcccctggcaaagaaagggaaagcgtggccagaatcagcagcggaggcggcacaatctactacgccgatagcgtgaagggcagattcaccatcagcagagaggacgccaagaacaccgtgtacctgcagatgaacagcctgaagcctgaggacaccgccgtgtactactgttacgtcggcggcttttggggccagggcacacaagtgacagtgtctagcggaggacagctccagctggtggaatctggtggtggactggttcaaccaggcggcagcctgaaactgtcttgtgccgcttctggcttcacattctcccgctacgccatgtcttggtataggcaggctcccggaaaagagagagagtccgtcgccagaatctcctctggcggcggaaccatctattatgccgactccgtgaaaggccggttcacaatctccagagaagatgccaaaaacacggtctatctccaaatgaactcactgaagcccgaagatacggccgtctactattgctatgttggcggattctggggacagggaacccaagtcactgtgtcctct

SEQ ID NO: 390 - DNA, 1266 bp, Artificial Sequence
  Description of Artificial Sequence Synthetic polynucleotide
atggactacaaggacgatgacgataaaggcagcagcggtcaccaccaccaccaccacgaaaacctgtatttccagggtatggaagcgggtcgtccgcgtccggtgctgcgtagcgttaacagccgtgagccgagccaagtgatcttctgcaaccgtagcccgcgtgtggttctgccggtttggctgaactttgatggtgaaccgcaaccgtacccgaccctgccgccgggtaccggtcgtcgtattcacagctatcgtggtcatctgtggctgttccgtgatgcgggtacccacgatggtctgctggtgaaccaaaccgagctgttcgtgccgagcctgaacgttgacggtcagccgatctttgcgaacattaccctgccggtttacaccctgaaggaacgttgcctgcaagtggttcgtagcctggtgaaaccggagaactaccgtcgtctggatatcgttcgtagcctgtatgaagacctggaggatcacccgaacgtgcagaaggacctggaacgtctgacccaagagcgtattgcgcaccagcgtatgggtgatggtggcggtggcggtgaatttgcgatggcgcaggtgcaactgcaagaaagcggcggtggcctggttcaaccgggtggcagcctgaagctgagctgcgcggcgagcggtttcacctttagccgttacgcgatgagctggtatcgtcaggcgccgggcaaagaacgtgagagcgttgcgcgtatcagcagcggtggcggtaccatttactatgcggacagcgtgaagggtcgtttcaccatcagccgtgaagatgcgaaaaacaccgtttacctgcaaatgaacagcctgaagccggaggacaccgcggtgtactattgctatgttggcggtttttggggccaaggtacccaggtgaccgttagcagcggcggtcaactgcagctggttgagagcggcggtggcctggtgcagccgggtggcagcctgaagctgtcttgcgcggcgagcggctttacctttagccgttatgcgatgagctggtaccgccaagcgccgggtaaagaacgtgagagcgtggcgcgcatcagcagcggtggcggtacgatctattatgcggacagcgtgaaaggtcgtttcaccattagccgtgaagatgcgaagaacaccgtttacctgcagatgaacagcctgaaaccggaggataccgcggtttattactgctacgttggcggtttttggggtcagggcacccaagttaccgttagcagc

SEQ ID NO: 391 - DNA, 900 bp, Artificial Sequence
  Description of Artificial Sequence Synthetic polynucleotide
tgccagttacaacttgtcgagtcaggcggtggcttggtacagcccggtggctctttaaaacttagttgtgctgcctcaggatttacgttctctaggtacgccatgtcatggtataggcaggcccccggcaaggaaagggagtcagttgctaggattagttccggcggaggaactatctattatgcagactctgtaaagggccgtttcaccatctctcgtgaggatgccaaaaacactgtgtacctacagatgaatagtttgaaacctgaggatacagctgtatattattgttatgtcggtggtttttggggccagggcacgcaagtcaccgtgtcctctgagcccaaaagtagtgataaaactcacacgtgtccaccctgccctggacaaccaagagagccccaggtatatactctaccaccatcacgtgaagagatgaccaagaaccaagtatcccttacctgtttggttaaaggcttctatccaagtgatattgccgtggaatgggagagtaatggtcaacccgaaaacaattacaagaccacccctcctgtcctggactccgacggaagtttttttctatacagtaaacttactgtggataagagtcgttggcaacaaggaaacgtgttctcctgctcagtgatgcacgaagcattacacaaccattatacgcagaaatctctatctcttagtcctggcggcggtggatctactttgccagtctataccctaaaagaacgttgtctgcaagtggtgaggagtttagtcaagcctgaaaactaccgtcgtttggacatagtaaggtcactatatgaagacttggaagatcaccccaacgtgcaaaaagatctagaaagactgacgcaggagagaatcgcacaccagaggatgggtgat

SEQ ID NO: 392 - protein, 130 aa, Artificial Sequence
  Description of Artificial Sequence Synthetic polypeptide
MHHHHHHENLYFQGEVQLLESGGGLVQPGGSLRLSCAASGFTFSTFSMNWVRQAPGKGLEWVSYISRTSKTIYYADSVKGRFTISRDNSKNTLYLQMNSLRAEDTAVYYCARGRFFDYWGQGTLVTVSSC

SEQ ID NO: 393 - protein, 129 aa, Artificial Sequence
  Description of Artificial Sequence Synthetic polypeptide
MHHHHHHENLYFQGEVQLLESGGGLVQPGGSLRLSCAASGFTFSTFSMNWVRQAPGKGLEWVSYISRTSKTIYYADSVKGRFTISRDNSKNTLYLQMNSLRAEDTAVYYCARGRFFDYWGQGTLVTVSS

SEQ ID NO: 394 - protein, 171 aa, Artificial Sequence
  Description of Artificial Sequence Synthetic polypeptide
MHHHHHHENLYFQGDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKLC

SEQ ID NO: 395 - protein, 170 aa, Artificial Sequence
  Description of Artificial Sequence Synthetic polypeptide
MHHHHHHENLYFQGDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKL

SEQ ID NO: 396 - protein, 139 aa, Artificial Sequence
  Description of Artificial Sequence Synthetic polypeptide
MHHHHHHENLYFQGEVQLVESGGGSVQTGGSLRLSCAVSGNIGSSYCMGWFRQAPGKKREAVARIVRDGATGYADYVKGRFTISRDSAKNTLYLQMNRLIPEDTAIYYCAADLPPGCLTQAIWNFGYRGQGTLVTVSSC

SEQ ID NO: 397 - protein, 138 aa, Artificial Sequence
  Description of Artificial Sequence Synthetic polypeptide
MHHHHHHENLYFQGEVQLVESGGGSVQTGGSLRLSCAVSGNIGSSYCMGWFRQAPGKKREAVARIVRDGATGYADYVKGRFTISRDSAKNTLYLQMNRLIPEDTAIYYCAADLPPGCLTQAIWNFGYRGQGTLVTVSS

SEQ ID NO: 398 - protein, 105 aa, Artificial Sequence
  Description of Artificial Sequence Synthetic polypeptide
VSDVPRKLEVVAATPTSLLISWDAPAVTVRYYRITYGETGGNSPVQEFTVPGSKSTATISGLKPGVDYTITVYAVTSGGGSFAEYWALLSGGGSPISINYRTALE

SEQ ID NO: 399 - protein, 127 aa, Artificial Sequence
  Description of Artificial Sequence Synthetic polypeptide
MHHHHHHENLYFQGQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSC

SEQ ID NO: 400 - protein, 126 aa, Artificial Sequence
  Description of Artificial Sequence Synthetic polypeptide
MHHHHHHENLYFQGQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSS

SEQ ID NO: 401 - protein, 131 aa, Artificial Sequence
  Description of Artificial Sequence Synthetic polypeptide
MHHHHHHENLYFQGQLQLVESGGGLVQPGGSLKLSCAASGFTFSRYAMSWYRQAPGKERESVARISSGGGTIYYADSVKGRFTISREDAKNTVYLQMNSLKPEDTAVYYCYVGGFWGQGTQVTVSSLPETG

SEQ ID NO: 402 - DNA, 402 bp, Artificial Sequence
  Description of Artificial Sequence Synthetic polynucleotide
atgcatcaccaccaccaccacgagaacctgtacttccagggcgaggtgcaactgctggagagcggtggcggtctggttcaaccgggcggtagcctgcgtctgagctgcgcggcgagcggtttcacctttagcacctttagcatgaactgggtgcgtcaagcgccgggcaagggtctggagtgggttagctatatcagccgtaccagcaagaccatttactatgcggacagcgtgaaaggccgtttcaccatcagccgtgataacagcaaaaacaccctgtacctgcagatgaacagcctgcgtgcggaagacaccgcggtttactattgcgcgcgtggtcgtttctttgattattggggccaaggtaccctggtgaccgttagcagctgctaatgaaagctt

SEQ ID NO: 403 - DNA, 399 bp, Artificial Sequence
  Description of Artificial Sequence Synthetic polynucleotide
atgcatcaccaccaccaccacgagaacctgtacttccagggcgaggtgcaactgctggagagcggtggcggtctggttcaaccgggcggtagcctgcgtctgagctgcgcggcgagcggtttcacctttagcacctttagcatgaactgggtgcgtcaagcgccgggcaagggtctggagtgggttagctatatcagccgtaccagcaagaccatttactatgcggacagcgtgaaaggccgtttcaccatcagccgtgataacagcaaaaacaccctgtacctgcagatgaacagcctgcgtgcggaagacaccgcggtttactattgcgcgcgtggtcgtttctttgattattggggccaaggtaccctggtgaccgttagcagctaatgaaagctt

SEQ ID NO: 404 - DNA, 525 bp, Artificial Sequence
  Description of Artificial Sequence Synthetic polynucleotide
atgcatcaccaccaccaccacgagaacctgtacttccagggtgacctgggcaagaaactgctggaggcggcgcgtgcgggtcaagacgatgaagtgcgtatcctgatggcgaacggtgcggacgttaacgcgagcgatcgttggggctggaccccgctgcacctggcggcgtggtggggtcacctggagattgtggaagttctgctgaaacgtggtgcggatgtgagcgcggcggatctgcacggccagagcccgctgcatctggcggcgatggtgggtcacctggagatcgtggaagttctgctgaagtatggcgcggatgttaacgcgaaagatacgatgggtgcgaccccgctgcatttagctgcgcgtagcggccacctggaaattgttgaggaactgctgaagaacggtgcggacatgaacgcgcaggataagttcggcaaaaccacctttgacatcagcaccgataacggtaacgaggatctggcggaaattctgcaaaaactgtgctaatgaaagctt

SEQ ID NO: 405 - DNA, 522 bp, Artificial Sequence
  Description of Artificial Sequence Synthetic polynucleotide
atgcatcaccaccaccaccacgagaacctgtacttccagggtgacctgggcaagaaactgctggaggcggcgcgtgcgggtcaagacgatgaagtgcgtatcctgatggcgaacggtgcggacgttaacgcgagcgatcgttggggctggaccccgctgcacctggcggcgtggtggggtcacctggagattgtggaagttctgctgaaacgtggtgcggatgtgagcgcggcggatctgcacggccagagcccgctgcatctggcggcgatggtgggtcacctggagatcgtggaagttctgctgaagtatggcgcggatgttaacgcgaaagatacgatgggtgcgaccccgctgcatttagctgcgcgtagcggccacctggaaattgttgaggaactgctgaagaacggtgcggacatgaacgcgcaggataagttcggcaaaaccacctttgacatcagcaccgataacggtaacgaggatctggcggaaattctgcaaaaactgtaatgaaagctt

SEQ ID NO: 406 - DNA, 417 bp, Artificial Sequence
  Description of Artificial Sequence Synthetic polynucleotide
atgcatcaccaccaccaccacgagaacctgtacttccagggcgaggtgcaactggttgaaagcggtggcggtagcgtgcagaccggcggtagcctgcgtctgagctgcgcggttagcggcaacatcggtagcagctattgcatgggctggttccgtcaagcgccgggtaagaaacgtgaagcggttgcgcgtattgttcgtgatggtgcgaccggttacgcggattatgtgaagggtcgttttaccattagccgtgacagcgcgaaaaacaccctgtacctgcagatgaaccgtctgatcccggaagacaccgcgatttactattgcgcggcggatctgccgccgggttgcctgacccaggcgatttggaactttggttatcgtggccaaggtaccctggtgaccgttagcagctgc

SEQ ID NO: 407 - DNA, 426 bp, Artificial Sequence
  Description of Artificial Sequence Synthetic polynucleotide
atgcatcaccaccaccaccacgagaacctgtacttccagggcgaggtgcaactggttgaaagcggtggcggtagcgtgcagaccggcggtagcctgcgtctgagctgcgcggttagcggcaacatcggtagcagctattgcatgggctggttccgtcaagcgccgggtaagaaacgtgaagcggttgcgcgtattgttcgtgatggtgcgaccggttacgcggattatgtgaagggtcgttttaccattagccgtgacagcgcgaaaaacaccctgtacctgcagatgaaccgtctgatcccggaagacaccgcgatttactattgcgcggcggatctgccgccgggttgcctgacccaggcgatttggaactttggttatcgtggccaaggtaccctggtgaccgttagcagctaatgaaagctt

SEQ ID NO: 408 - DNA, 372 bp, Artificial Sequence
  Description of Artificial Sequence Synthetic polynucleotide
gcacaagtccaattagtagaaagtggaggaggtttagttcagccaggcggcagtctgactctgagttgtaccgcctcaggcttcacgctagaccattatgacataggttggttcagacaggctccaggcaaagagcgtgaaggagtctcttgcataaacaactccgacgacgacacatattatgccgactcagtgaagggccgtttcactatatttatgaataacgccaaggatacggtttaccttcagatgaacagtctgaagccagaagatactgctatatattactgtgctgaagcccgtggttgcaagcgtggaagatacgaatatgacttttggggacaaggaacgcaagtcacggtcagttcttgc

SEQ ID NO: 409 - DNA, 329 bp, Artificial Sequence
  Description of Artificial Sequence Synthetic polynucleotide
atatggtgagcgacgtgccgcgtaaactggaagtggttgcggcgaccccgaccagcctgctgattagctgggatgcgccggcggtgaccgtgcgttactatcgtatcacctacggcgagaccggtggcaacagcccggtgcaggaattcaccgttccgggtagcaagagcaccgcgaccatcagcggcctgaaaccgggtgtggactacaccattaccgtgtatgcggttaccagcggtggcggtagctttgcggagtattgggcgctgctgagcggcggcggtagcccgattagcattaactatcgcaccgcgctggaataaggatcc

SEQ ID NO: 410 - DNA, 369 bp, Artificial Sequence
  Description of Artificial Sequence Synthetic polynucleotide
catatggtgagcgacgtgccgcgtaaactggaagtggttgcggcgaccccgaccagcctgctgattagctgggatgcgccggcggtgaccgtgcgttactatcgtatcacctacggcgagaccagcggtggcggtagcttcgcggaatattgggcgctgctgagcggcggtggcagcgtgcaggagtttaccgttccgggtagcaagagcaccgcgaccatcagcggcctgaaaccgggtgtggactacaccattaccgtgtatgcggttaccagcggtggcggtagctttgcggaatactgggcgctgctgagcggcggcggtagcccgattagcattaactatcgcaccgcgctggaataaggatcc

SEQ ID NO: 411 - DNA, 381 bp, Artificial Sequence
  Description of Artificial Sequence Synthetic polynucleotide
atgcatcaccaccaccaccacgagaacctgtacttccagggtcaactgcagctggttgagagcggtggcggtctggttcaaccgggcggtagcctgaaactgagctgcgcggcgagcggtttcacctttagccgttacgcgatgagctggtatcgtcaagcgccgggcaaggagcgtgaaagcgtggcgcgtatcagcagcggcggtggcaccatttactatgcggacagcgtgaagggccgtttcaccatcagccgtgaggatgcgaaaaacaccgtttacctgcaaatgaacagcctgaagccggaagacaccgcggtgtactattgctatgttggtggcttttggggtcaaggcacccaggtgaccgttagcagctgc

SEQ ID NO: 412 - DNA, 378 bp, Artificial Sequence
  Description of Artificial Sequence Synthetic polynucleotide
atgcatcaccaccaccaccacgagaacctgtacttccagggtcaactgcagctggttgagagcggtggcggtctggttcaaccgggcggtagcctgaaactgagctgcgcggcgagcggtttcacctttagccgttacgcgatgagctggtatcgtcaagcgccgggcaaggagcgtgaaagcgtggcgcgtatcagcagcggcggtggcaccatttactatgcggacagcgtgaagggccgtttcaccatcagccgtgaggatgcgaaaaacaccgtttacctgcaaatgaacagcctgaagccggaagacaccgcggtgtactattgctatgttggtggcttttggggtcaaggcacccaggtgaccgttagcagc

SEQ ID NO: 413 - DNA, 393 bp, Artificial Sequence
  Description of Artificial Sequence Synthetic polynucleotide
atgcatcaccaccaccaccacgagaacctgtacttccagggtcaactgcagctggttgagagcggtggcggtctggttcaaccgggcggtagcctgaaactgagctgcgcggcgagcggtttcacctttagccgttacgcgatgagctggtatcgtcaagcgccgggcaaggagcgtgaaagcgtggcgcgtatcagcagcggcggtggcaccatttactatgcggacagcgtgaagggtcgtttcaccatcagccgtgaggatgcgaaaaacaccgtttacctgcaaatgaacagcctgaagccggaagacaccgcggtgtactattgctatgttggtggcttttggggtcaaggcacccaggtgaccgttagcagcctgccggagaccggc

SEQ ID NO: 414 - DNA, 339 bp, Artificial Sequence
  Description of Artificial Sequence Synthetic polynucleotide
caactgcagctggttgaatctggcggcggactggttcaacctggcggatctctgaagctgagctgtgccgccagcggcttcacctttagcagatacgccatgagctggtacagacaggcccctggcaaagaaagggaaagcgtggccagaatcagcagcggcggaggcacaatctactacgccgatagcgtgaagggcagattcaccatcagcagagaggacgccaagaacaccgtgtacctgcagatgaacagcctgaagcctgaggacaccgccgtgtactactgttacgtcggcggcttttggggccagggcacacaagtgacagtgtcctcttgt

SEQ ID NO: 415 - DNA, 336 bp, Artificial Sequence
  Description of Artificial Sequence Synthetic polynucleotide
caactgcagctggttgaatctggcggcggactggttcaacctggcggatctctgaagctgagctgtgccgccagcggcttcacctttagcagatacgccatgagctggtacagacaggcccctggcaaagaaagggaaagcgtggccagaatcagcagcggcggaggcacaatctactacgccgatagcgtgaagggcagattcaccatcagcagagaggacgccaagaacaccgtgtacctgcagatgaacagcctgaagcctgaggacaccgccgtgtactactgttacgtcggcggcttttggggccagggcacacaagtgacagtgtcctct

SEQ ID NO: 416 - DNA, 351 bp, Artificial Sequence
  Description of Artificial Sequence Synthetic polynucleotide
caactgcagctggttgaatctggcggcggactggttcaacctggcggatctctgaagctgagctgtgccgccagcggcttcacctttagcagatacgccatgagctggtacagacaggcccctggcaaagaaagggaaagcgtggccagaatcagcagcggcggaggcacaatctactacgccgatagcgtgaagggcagattcaccatcagcagagaggacgccaagaacaccgtgtacctgcagatgaacagcctgaagcctgaggacaccgccgtgtactactgttacgtcggcggcttttggggccagggcacacaagtgacagtgtccagcctgcctgagacagga

SEQ ID NO: 417 - DNA, 339 bp, Artificial Sequence
  Description of Artificial Sequence Synthetic polynucleotide
caactacagcttgttgagtctggaggcggccttgtccagcctggtggctcacttaagttatcatgtgctgcatctggatttaccttctccaggtatgcaatgtcttggtataggcaggcacctggtaaagaaagggaatccgttgcaagaatcagttcaggtggcggcacgatatactacgccgactcagtcaaaggtcgtttcacaatatccagggaggatgccaaaaatacggtttatttgcaaatgaatagtttgaaacccgaagatacagcagtgtactactgctacgtcggaggcttctggggacaaggcacacaagtcaccgtttccagttgc

SEQ ID NO: 418 - protein, 20 aa, Artificial Sequence
  Description of Artificial Sequence Synthetic peptide
AVKRPAATKKAGQAKKKKLD

SEQ ID NO: 419 - protein, 9 aa, Artificial Sequence
  Description of Artificial Sequence Synthetic peptide
KLKIKRPVK

SEQ ID NO: 420 - protein, 25 aa, Artificial Sequence
  Description of Artificial Sequence Synthetic peptide
MSRRRKANPTKLSENAKKLAKEVEN

SEQ ID NO: 421 - protein, 41 aa, Artificial Sequence
  Description of Artificial Sequence Synthetic polypeptide
RMRKFKNKGKDTAELRRRRVEVSVELRKAKKDEQILKRRNV

SEQ ID NO: 422 - protein, 420 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDYKDDDDKMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKC

SEQ ID NO: 423 - protein, 420 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDYKDDDDKMTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKC

SEQ ID NO: 424 - protein, 420 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDYKDDDDKMTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKC

SEQ ID NO: 425 - protein, 419 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDYKDDDDKMTEYKLVVVGAGDVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKK

SEQ ID NO: 426 - protein, 423 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDYKDDDDKMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 427 - protein, 423 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDYKDDDDKMTEYKLVVVGAVGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 428 - protein, 423 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSDYKDDDDKMTEYKLVVVGADGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 429 - DNA, 600 bp, Artificial Sequence
  Description of Artificial Sequence Synthetic polynucleotide
ggatccgactacaaggacgatgacgataaaatgaccgagtataaactggtggtggtgggtgcgggtggcgttggtaaaagcgcgctgaccatccagctgattcaaaaccacttcgtggacgagtacgatccgaccatcgaagacagctatcgtaagcaggttgtgatcgatggcgagacctgcctgctggacattctggataccgcgggccaggaagagtacagcgcgatgcgtgaccaatatatgcgtaccggtgaaggcttcctgtgcgtttttgcgattaacaacaccaaaagctttgaggatatccaccactaccgtgaacaaattaagcgtgttaaagacagcgaggatgtgccgatggttctggtgggtaacaaatgcgacctgccgagccgtaccgtggacaccaagcaggcgcaagatctggcgcgtagctatggcatcccgttcattgaaaccagcgcgaaaacccgtcagcgtgttgaggatgcgttttacaccctggtgcgtgaaatccgtcaatatcgtctgaagaaaattagcaaagaggagaaaaccccgggttgcgttaagatcaagaaatgctaatgactcgag

SEQ ID NO: 430 - DNA, 594 bp, Artificial Sequence
  Description of Artificial Sequence Synthetic polynucleotide
ggatccgactacaaggacgatgacgataaaatgaccgagtataaactggtggtggtgggtgcggttggcgttggtaaaagcgcgctgaccatccagctgattcaaaaccacttcgtggacgagtacgatccgaccatcgaagacagctatcgtaagcaggttgtgatcgatggcgagacctgcctgctggacattctggataccgcgggccaggaagagtacagcgcgatgcgtgaccaatatatgcgtaccggtgaaggcttcctgtgcgtttttgcgattaacaacaccaaaagctttgaggatatccaccactaccgtgaacaaattaagcgtgttaaagacagcgaggatgtgccgatggttctggtgggtaacaaatgcgacctgccgagccgtaccgtggacaccaagcaggcgcaagatctggcgcgtagctatggcatcccgttcattgaaaccagcgcgaaaacccgtcagcgtgttgaggatgcgttttacaccctggtgcgtgaaatccgtcaatatcgtctgaagaaaattagcaaagaggagaaaaccccgggttgcgttaagatcaagaaatgctaatga

SEQ ID NO: 431 - DNA, 600 bp, Artificial Sequence
  Description of Artificial Sequence Synthetic polynucleotide
ggatccgactacaaggacgatgacgataaaatgaccgagtataaactggtggtggtgggtgcggatggcgttggtaaaagcgcgctgaccatccagctgattcaaaaccacttcgtggacgagtacgatccgaccatcgaagacagctatcgtaagcaggttgtgatcgatggcgagacctgcctgctggacattctggataccgcgggccaggaagagtacagcgcgatgcgtgaccaatatatgcgtaccggtgaaggcttcctgtgcgtttttgcgattaacaacaccaaaagctttgaggatatccaccactaccgtgaacaaattaagcgtgttaaagacagcgaggatgtgccgatggttctggtgggtaacaaatgcgacctgccgagccgtaccgtggacaccaagcaggcgcaagatctggcgcgtagctatggcatcccgttcattgaaaccagcgcgaaaacccgtcagcgtgttgaggatgcgttttacaccctggtgcgtgaaatccgtcaatatcgtctgaagaaaattagcaaagaggagaaaaccccgggttgcgttaagatcaagaaatgctaatgactcgag

SEQ ID NO: 432 - DNA, 600 bp, Artificial Sequence
  Description of Artificial Sequence Synthetic polynucleotide
ggatccgactacaaggacgatgacgataaaatgaccgagtataaactggtggtggtgggtgcgggtgatgttggtaaaagcgcgctgaccatccagctgattcaaaaccacttcgtggacgagtacgatccgaccatcgaagacagctatcgtaagcaggttgtgatcgatggcgagacctgcctgctggacattctggataccgcgggccaggaagagtacagcgcgatgcgtgaccaatatatgcgtaccggtgaaggcttcctgtgcgtttttgcgattaacaacaccaaaagctttgaggatatccaccactaccgtgaacaaattaagcgtgttaaagacagcgaggatgtgccgatggttctggtgggtaacaaatgcgacctgccgagccgtaccgtggacaccaagcaggcgcaagatctggcgcgtagctatggcatcccgttcattgaaaccagcgcgaaaacccgtcagcgtgttgaggatgcgttttacaccctggtgcgtgaaatccgtcaatatcgtctgaagaaaattagcaaagaggagaaaaccccgggttgcgttaagatcaagaaatgctaatgactcgag

SEQ ID NO: 433 - DNA, 609 bp, Artificial Sequence
  Description of Artificial Sequence Synthetic polynucleotide
ggatccgactacaaggacgatgacgataaaatgaccgagtataaactggtggtggtgggtgcgggtggcgttggtaaaagcgcgctgaccatccagctgattcaaaaccacttcgtggacgagtacgatccgaccatcgaagacagctatcgtaagcaggttgtgatcgatggcgagacctgcctgctggacattctggataccgcgggccaggaagagtacagcgcgatgcgtgatcaatatatgcgtaccggtgaaggcttcctgtgcgtttttgcgattaacaacaccaaaagctttgaggacatccaccagtaccgtgaacaaattaagcgtgttaaagatagcgacgatgtgccgatggttctggtgggtaacaagtgcgacctggcggcgcgtaccgtggagagccgtcaggcgcaagatctggcgcgtagctacggtatcccgtatattgaaaccagcgcgaaaacccgtcagggcgttgaggacgcgttctataccctggtgcgtgaaatccgtcaacacaagctgcgtaaactgaacccgccggatgaaagcggtccgggctgcatgagctgcaagtgcgttctgagctaatgactcgag

SEQ ID NO: 434 - DNA, 609 bp, Artificial Sequence
  Description of Artificial Sequence Synthetic polynucleotide
ggatccgactacaaggacgatgacgataaaatgaccgagtataaactggtggtggtgggtgcggttggcgttggtaaaagcgcgctgaccatccagctgattcaaaaccacttcgtggacgagtacgatccgaccatcgaagacagctatcgtaagcaggttgtgatcgatggcgagacctgcctgctggacattctggataccgcgggccaggaagagtacagcgcgatgcgtgatcaatatatgcgtaccggtgaaggcttcctgtgcgtttttgcgattaacaacaccaaaagctttgaggacatccaccagtaccgtgaacaaattaagcgtgttaaagatagcgacgatgtgccgatggttctggtgggtaacaagtgcgacctggcggcgcgtaccgtggagagccgtcaggcgcaagatctggcgcgtagctacggtatcccgtatattgaaaccagcgcgaaaacccgtcagggcgttgaggacgcgttctataccctggtgcgtgaaatccgtcaacacaagctgcgtaaactgaacccgccggatgaaagcggtccgggctgcatgagctgcaagtgcgttctgagctaatgactcgag

SEQ ID NO: 435 - DNA, 609 bp, Artificial Sequence
  Description of Artificial Sequence Synthetic polynucleotide
ggatccgactacaaggacgatgacgataaaatgaccgagtataaactggtggtggtgggtgcggatggcgttggtaaaagcgcgctgaccatccagctgattcaaaaccacttcgtggacgagtacgatccgaccatcgaagacagctatcgtaagcaggttgtgatcgatggcgagacctgcctgctggacattctggataccgcgggccaggaagagtacagcgcgatgcgtgatcaatatatgcgtaccggtgaaggcttcctgtgcgtttttgcgattaacaacaccaaaagctttgaggacatccaccagtaccgtgaacaaattaagcgtgttaaagatagcgacgatgtgccgatggttctggtgggtaacaagtgcgacctggcggcgcgtaccgtggagagccgtcaggcgcaagatctggcgcgtagctacggtatcccgtatattgaaaccagcgcgaaaacccgtcagggcgttgaggacgcgttctataccctggtgcgtgaaatccgtcaacacaagctgcgtaaactgaacccgccggatgaaagcggtccgggctgcatgagctgcaagtgcgttctgagctaatgactcgag

SEQ ID NO: 436 - protein, 816 aa, Artificial Sequence
  Description of Artificial Sequence Synthetic polypeptide
MGSSHHHHHHSSGENLYFQGHMASMTGGEEMGRGSMATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 437 - DNA, 2354 bp, Artificial Sequence
  Description of Artificial Sequence Synthetic polynucleotide
atggctacacaagcggacctgatggagctggatatggcgatggagccggatcgtaaggcggcggttagccactggcagcaacagagctacctggatagcggcatccacagcggtgcgaccaccaccgcgccgagcctgagcggtaaaggcaacccggaggaagaggacgttgataccagccaggtgctgtacgaatgggagcagggcttcagccagagcttcacccaggagcaagttgcggacatcgatggccagtatgcgatgacccgtgcgcaacgtgtgcgtgcggcgatgttcccggaaaccctggatgagggtatgcagattccgagcacccaatttgacgcggcgcacccgaccaacgttcagcgtctggcggaaccgagccaaatgctgaagcacgcggtggttaacctgatcaactatcaggacgatgcggaactggcgacccgtgcgattccggagctgaccaagctgctgaacgacgaagatcaggtggttgtgaacaaagcggcggtgatggttcaccaactgagcaagaaagaggcgagccgtcacgcgatcatgcgtagcccgcagatggttagcgcgattgtgcgtaccatgcaaaacaccaacgatgtggaaaccgcgcgttgcaccgcgggtaccctgcacaacctgagccaccaccgtgaaggtctgctggcgatcttcaagagcggtggcattccggcgctggttaaaatgctgggcagcccggtggacagcgttctgttttacgcgatcaccaccctgcacaacctgctgctgcaccaggaaggtgcgaaaatggcggtgcgtctggcgggtggcctgcaaaaaatggttgcgctgctgaacaagaccaacgtgaaattcctggcgatcaccaccgactgcctgcagattctggcgtatggcaaccaagagagcaagctgatcattctggcgagcggtggcccgcaggcgctggttaacatcatgcgtacctacacctatgaaaaactgctgtggaccaccagccgtgtgctgaaggttctgagcgtgtgcagcagcaacaaaccggcgattgttgaggcgggtggcatgcaagcgctgggtctgcacctgaccgatccgagccagcgtctggtgcaaaactgcctgtggaccctgcgtaacctgagcgacgcggcgaccaaacaggaaggtatggagggcctgctgggtaccctggtgcaactgctgggcagcgacgatatcaacgttgtgacctgcgcggcgggtattctgagcaacctgacctgcaacaactacaagaacaaaatgatggtttgccaagtgggtggcatcgaggcgctggttcgtaccgtgctgcgtgcgggcgaccgtgaagatatcaccgagccggcgatttgcgcgctgcgtcacctgaccagccgtcaccaggaagcggagatggcgcaaaacgcggttcgtctgcactatggtctgccggttgtggttaagctgctgcacccgccgagccactggccgctgatcaaagcgaccgtgggcctgattcgtaacctggcgctgtgcccggcgaaccatgcgccgctgcgtgaacagggtgcgattccgcgtctggttcaactgctggtgcgtgcgcaccaggatacccaacgtcgtaccagcatgggtggcacccaacagcaattcgttgagggtgtgcgtatggaagagatcgttgaaggctgcaccggtgcgctgcacattctggcgcgtgacgttcacaaccgtatcgtgattcgtggcctgaacaccatcccgctgtttgtgcagctgctgtacagcccgatcgagaacattcaacgtgttgcggcgggtgtgctgtgcgaactggcgcaggataaggaagcggcggaggcgattgaggcggagggtgcgaccgcgccgctgaccgaactgctgcacagccgtaacgagggtgttgcgacctacgcggcggcggtgctgttccgtatgagcgaggacaaaccgcaggattataagaaacgtctgagcgttgaactgaccagcagcctgtttcgtaccgaaccgatggcgtggaacgaaaccgcggacctgggcctggatattggcgcgcagggtgaaccgctgggttaccgtcaagacgatccgagctatcgtagctttcatagcggtggctacggtcaggatgcgctgggtatggacccgatgatggaacatgagatgggtggccatcacccgggtgcggactatccggtggatggcctgccggacctgggtcacgcgcaagacctgatggatggcctgccgccgggcgatagcaatcaactggcgtggtttgacaccgacctgtaatgagaatt

SEQ ID NO: 438 - protein, 352 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSMCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVV

SEQ ID NO: 439 - protein, 420 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSMTSFSTSAQCSTSDSACRISPGQINQVRPKLPLLKILHAAGAQGEMFTVKEVMHYLGQYIMVKQLYDQQEQHMVYCGGDLLGELLGRQSFSVKDPSPLYDMLRKNLVTLATATTDAAQTLALAQDHSMDIPSQDQLKQSAEESSTSRKRTTEDDIPTLPTSEHKCIHSREDEDLIENL

SEQ ID NO: 440 - DNA, 1056 bp, Artificial Sequence
  Description of Artificial Sequence Synthetic polynucleotide
atgagcccaattctgggttactggaagatcaaaggtctggttcagccgacccgtctgctgctggaatatctggaagagaaatatgaagaacacctgtatgagcgtgatgaaggtgataaatggcgtaacaagaaatttgaactgggtctggaatttcctaacctgccgtactacattgacggtgacgtgaaactgacccagtccatggcaatcatccgttacattgcagacaaacataacatgctgggtggttgcccgaaagaacgtgcggaaatttccatgctggaaggtgcggtcctggacattcgttatggcgtaagccgtatcgcttactctaaagattttgagaccctgaaagttgattttctgagcaaactgccggaaatgctgaaaatgttcgaggaccgcctgtgccacaaaacttacctgaacggtgatcacgttacccaccctgacttcatgctgtacgatgctctggacgttgtactgtacatggacccgatgtgcctggacgcatttccgaagctggtttgctttaaaaaacgcatcgaagcgatcccgcaaattgacaaatatctgaaaagctctaaatacattgcttggccactgcagggttggcaggctaccttcggtggtggtgaccacccacctaaaagcgatggctctacttccggctccggccatcaccaccatcaccactctgctggcctggttcctcgtggttctatgtgtaacaccaatatgtctgtgccgaccgacggtgcagtgactacctcccagatcccggcgtccgaacaggagaccctggttcgtccgaaaccgctgctgctgaaactgctgaaaagcgtaggtgcgcagaaagacacctataccatgaaagaagtgctgttttatctgggccagtatatcatgactaagcgtctgtacgacgaaaaacagcagcatatcgtatactgttctaacgacctgctgggtgacctgttcggcgtaccaagcttctctgttaaagaacaccgtaaaatctatactatgatttaccgcaacctggtagtagtg

SEQ ID NO: 441 - DNA, 1260 bp, Artificial Sequence
  Description of Artificial Sequence Synthetic polynucleotide
atgtctccaatcctgggctactggaaaatcaaaggcctggtgcaaccaacccgtctgctgctggaatatctggaagaaaaatacgaggaacatctgtatgaacgtgacgaaggcgacaaatggcgcaacaaaaaatttgaactgggtctggagttcccgaacctgccgtactacatcgatggtgacgttaaactgacgcaaagcatggcgattattcgttacattgccgataaacacaacatgctgggtggctgtccgaaggaacgtgctgaaatctctatgctggaaggtgcagtgctggatattcgttacggtgtttctcgcattgcctactccaaggacttcgagaccctgaaagtcgactttctgtctaaactgccggaaatgctgaaaatgttcgaagaccgtctgtgccacaaaacctacctgaacggcgatcacgtcacccatccggacttcatgctgtacgacgctctggacgttgtgctgtatatggatcctatgtgcctggacgccttcccgaaactggtatgcttcaagaaacgtatcgaagctattccgcagatcgataaatacctgaaatcttctaaatacattgcttggccgctgcagggttggcaagcaacctttggcggtggcgatcaccctccgaaatccgacggctccacttctggctctggtcaccaccaccaccaccactccgcaggtctggtccctcgtggttccatgacttctttttccacttctgctcagtgttctaccagcgatagcgcgtgccgtatctccccgggtcagatcaaccaggtacgtccgaaactgccgctgctgaagatcctgcatgctgctggcgcgcaaggcgaaatgtttaccgttaaagaagtgatgcactacctgggtcagtacatcatggtcaaacagctgtatgatcaacaggaacagcatatggtatattgcggtggtgatctgctgggtgaactgctgggtcgccaatctttttccgttaaagatccttctcctctgtatgacatgctgcgcaaaaacctggtgaccctggcgaccgcaactactgacgctgcacaaactctggccctggcccaagaccactctatggacattccgtctcaggatcaactgaagcagtccgccgaggaaagcagcaccagccgtaaacgtaccaccgaggatgatattccgaccctgccgaccagcgaacacaaatgcattcacagccgcgaagacgaggacctgatcgaaaatctg

SEQ ID NO: 442 - protein, 1050 aa, Artificial Sequence
  Description of Artificial Sequence Synthetic polypeptide
MHHHHHHDYKDDDDKKMASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLWLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 443 - DNA, 3162 bp, Artificial Sequence
  Description of Artificial Sequence Synthetic polynucleotide
atgcatcaccaccaccaccacgattacaaggatgatgatgataagaagatggcgagcacccgttgcaagctggcgcgttatctggaagacctggaggacgttgatctgaagaaattcaaaatgcacctggaagattacccgccgcagaagggttgcatcccgctgccgcgtggccaaaccgagaaagcggaccacgttgatctggcgaccctgatgattgacttcaacggcgaggaaaaggcgtgggcgatggcggtgtggatctttgcggcgattaaccgtcgtgatctgtatgaaaaggcgaaacgtgacgagccgaaatggggtagcgataacgcgcgtgtgagcaacccgaccgttatctgccaggaagacagcattgaggaagagtggatgggcctgctggaatacctgagccgtatcagcatttgcaagatgaagaaagactaccgtaagaaataccgtaagtatgttcgtagccgtttccagtgcatcgaggatcgtaacgcgcgtctgggtgaaagcgtgagcctgaacaaacgttacacccgtctgcgtctgattaaggagcaccgtagccagcaagagcgtgaacaagagctgctggcgatcggcaagaccaaaacctgcgagagcccggttagcccgattaagatggaactgctgttcgacccggacgatgaacacagcgagccggtgcacaccgtggtttttcagggtgcggcgggtatcggcaagaccattctggcgcgtaaaatgatgctggattgggcgagcggcaccctgtaccaggaccgtttcgattacctgttttatatccactgccgtgaagtgagcctggttacccaacgtagcctgggtgacctgattatgagctgctgcccggatccgaacccgccgatccacaagattgttcgtaaaccgagccgtatcctgttcctgatggacggttttgatgagctgcagggcgcgttcgacgaacacattggtccgctgtgcaccgactggcaaaaagcggagcgtggcgatatcctgctgagcagcctgattcgtaagaaactgctgccggaggcgagcctgctgatcaccacccgtccggtggcgctggaaaaactgcagcacctgctggaccacccgcgtcacgttgagattctgggttttagcgaagcgaagcgtaaagagtacttctttaagtatttcagcgatgaagcgcaggcgcgtgcggcgtttagcctgatccaagaaaacgaggtgctgttcaccatgtgctttatcccgctggtgtgctggattgtttgcaccggtctgaagcagcaaatggaaagcggcaaaagcctggcgcagaccagcaagaccaccaccgcggtgtacgttttctttctgagcagcctgctgcaaccgcgtggtggcagccaagaacatggtctgtgcgcgcacctgtggggcctgtgcagcctggcggcggacggtatctggaaccaaaaaattctgttcgaagagagcgacctgcgtaaccacggcctgcagaaggcggatgtgagcgcgttcctgcgtatgaacctgtttcaaaaggaagttgattgcgagaaattctatagctttatccacatgacctttcaggaattctttgcggcgatgtactatctgctggaagaggaaaaagagggtcgtaccaacgtgccgggtagccgtctgaagctgccgagccgtgacgtgaccgttctgctggaaaactacggcaagttcgagaaaggctatctgatttttgtggttcgtttcctgtttggtctggtgaaccaggaacgtaccagctacctggagaagaaactgagctgcaaaatcagccagcaaattcgtctggaactgctgaagtggatcgaggttaaggcgaaagcgaagaaactgcagattcaaccgagccaactggagctgttctactgcctgtatgaaatgcaggaagaggacttcgtgcaacgtgcgatggattattttccgaagatcgaaattaacctgagcacccgtatggaccacatggttagcagcttctgcatcgaaaactgccaccgtgttgagagcctgagcctgggttttctgcacaacatgccgaaagaggaagaggaagaggaaaaagagggtagacacctggatatggtgcaatgcgttctgccgagcagcagccatgcggcgtgcagccacggtctggtgaacagccacctgaccagcagcttctgccgtggcctgtttagcgttctgagcaccagccagagcctgaccgaactggacctgagcgataacagcctgggtgacccgggcatgcgtgtgctgtgcgagaccctgcaacacccgggttgcaacatccgtcgtctgtggctgggtcgttgcggcctgagccacgaatgctgcttcgacattagcctggtgctgagcagcaaccaaaaactggttgagctggatctgagcgacaacgcgctgggtgattttggcatccgtctgctgtgcgtgggtctgaagcacctgctgtgcaacctgaagaaactgtggctggttagctgctgcctgaccagcgcgtgctgccaggacctggcgagcgtgctgagcaccagccacagcctgacccgtctgtacgtgggcgaaaacgcgctgggtgatagcggcgttgcgatcctgtgcgagaaggcgaaaaacccgcagtgcaacctgcaaaaactgggtctggtgaacagcggtctgaccagcgtttgctgcagcgcgctgagcagcgttctgagcaccaaccagaacctgacccacctgtatctgcgtggtaacaccctgggtgacaagggcatcaaactgctgtgcgaaggcctgctgcacccggactgcaagctgcaggtgctggagctggataactgcaacctgaccagccactgctgctgggatctgagcaccctgctgaccagcagccaaagcctgcgtaaactgagcctgggtaacaacgacctgggtgatctgggcgtgatgatgttctgcgaggttctgaagcagcaaagctgcctgctgcagaacctgggcctgagcgaaatgtactttaactatgagaccaaaagcgcgctggaaaccctgcaagaggaaaagccggagctgaccgtggttttcgaaccgagctggtaatgaaagctt

SEQ ID NO: 444 - protein, 1047 aa, Artificial Sequence
  Description of Artificial Sequence Synthetic polypeptide
MHHHHHHDYKDDDDKTSVRCKLAQYLEDLEDVDLKKFKMHLEDYPPEKGCIPVPRGQMEKADHLDLATLMIDFNGEEKAWAMAVWIFAAINRRDLWEKAKKDQPEWNDTCTSHSSMVCQEDSLEEEWMGLLGYLSRISICKKKKDYCKMYRRHVRSRFYSIKDRNARLGESVDLNSRYTQLQLVKEHPSKQEREHELLTIGRTKMRDSPMSSLKLELLFEPEDGHSEPVHTVVFQGAAGIGKTILARKIMLDWALGKLFKDKFDYLFFIHCREVSLRTPRSLADLIVSCWPDPNPPVCKILRKPSRILFLMDGFDELQGAFDEHIGEVCTDWQKAVRGDILLSSLIRKKLLPKASLLITTRPVALEKLQHLLDHPRHVEILGFSEAKRKEYFFKYFSNELQAREAFRLIQENEVLFTMCFIPLVCWIVCTGLKQQMETGKSLAQTSKTTTAVYVFFLSSLLQSRGGIEEHLFSDYLQGLCSLAADGIWNQKILFEECDLRKHGLQKTDVSAFLRMNVFQKEVDCERFYSFSHMTFQEFFAAMYYLLEEEAEGETVRKGPGGCSDLLNRDVKVLLENYGKFEKGYLIFVVRFLFGLVNQERTSYLEKKLSCKISQQVRLELLKWIEVKAKAKKLQWQPSQLELFYCLYEMQEEDFVQSAMDHFPKIEINLSTRMDHVVSSFCIKNCHRVKTLSLGFFHNSPKEEEEERRGGRPLDQVQCVFPDTHVACSSRLVNCCLTSSFCRGLFSSLSTNRSLTELDLSDNTLGDPGMRVLCEALQHPGCNIQRLWLGRCGLSHQCCFDISSVLSSSQKLVELDLSDNALGDFGIRLLCVGLKHLLCNLQKLWLVSCCLTSACCQDLALVLSSNHSLTRLYIGENALGDSGVQVLCEKMKDPQCNLQKLGLVNSGLTSICCSALTSVLKTNQNFTHLYLRSNALGDTGLRLLCEGLLHPDCKLQMLELDNCSLTSHSCWNLSTILTHNHSLRKLNLGNNDLGDLCVVTLCEVLKQQGCLLQSLQLGEMYLNRETKRALEALQEEKPELTIVFEISW

SEQ ID NO: 445 - DNA, 3153 bp, Artificial Sequence
  Description of Artificial Sequence Synthetic polynucleotide
atgcatcaccaccaccaccacgattacaaggatgatgatgataagacaagcgtgcgttgcaaactggcgcagtatctggaggacctggaagacgttgatctgaagaaattcaaaatgcacctggaggattacccgccggaaaagggttgcatcccggttccgcgtggccaaatggaaaaagcggaccacctggatctggcgaccctgatgattgacttcaacggcgaggaaaaggcgtgggcgatggcggtgtggatctttgcggcgattaaccgtcgtgatctgtgggaaaaggcgaagaaagaccagccggagtggaacgatacctgcaccagccacagcagcatggtttgccaagaggacagcctggaggaagagtggatgggtctgctgggctatctgagccgtatcagcatttgcaagaaaaagaaagattactgcaagatgtaccgtcgtcacgtgcgtagccgtttctacagcatcaaggaccgtaacgcgcgtctgggtgaaagcgtggatctgaacagccgttatacccagctgcaactggttaaggagcacccgagcaaacaggaacgtgagcacgaactgctgaccattggccgtaccaaaatgcgtgacagcccgatgagcagcctgaagctggaactgctgttcgagccggaagatggtcacagcgagccggttcacaccgtggtttttcagggcgcggcgggtatcggcaagaccattctggcgcgtaaaatcatgctggactgggcgctgggtaaactgttcaaggacaaatttgattacctgttctttatccactgccgtgaagtgagcctgcgtaccccgcgtagcctggcggacctgattgttagctgctggccggatccgaacccgccggtttgcaagatcctgcgtaaaccgagccgtattctgttcctgatggacggttttgatgagctgcagggcgcgttcgacgagcacattggtgaagtgtgcaccgactggcaaaaagcggttcgtggcgatatcctgctgagcagcctgattcgtaagaaactgctgccgaaagcgagcctgctgatcaccacccgtccggtggcgctggaaaagctgcagcacctgctggatcacccgcgtcacgttgagattctgggttttagcgaggcgaagcgtaaggaatacttctttaagtacttcagcaacgagctgcaggcgcgtgaagcgtttcgtctgatccaagagaacgaagttctgttcaccatgtgctttatcccgctggtgtgctggattgtttgcaccggtctgaagcagcaaatggaaaccggcaaaagcctggcgcagaccagcaagaccaccaccgcggtgtacgttttctttctgagcagcctgctgcaaagccgtggtggcattgaagagcacctgttcagcgactatctgcagggtctgtgcagcctggcggcggatggcatctggaaccaaaaaattctgtttgaagagtgcgacctgcgtaagcacggtctgcagaaaaccgatgtgagcgcgttcctgcgtatgaacgtgtttcaaaaagaggtggactgcgaacgtttctacagctttagccacatgaccttccaggagttctttgcggcgatgtactatctgctggaagaggaagcggagggtgaaaccgttcgtaaaggtccgggtggctgcagcgacctgctgaaccgtgatgtgaaggttctgctggagaactacggcaagttcgaaaaaggctatctgatctttgtggttcgtttcctgtttggcctggtgaaccaggagcgtaccagctatctggaaaagaaactgagctgcaaaatcagccagcaagtgcgtctggagctgctgaagtggattgaagttaaggcgaaagcgaagaaactgcagtggcaaccgagccaactggaactgttctactgcctgtatgagatgcaggaagaggacttcgttcaaagcgcgatggatcactttccgaaaatcgaaattaacctgagcacccgtatggaccacgtggttagcagcttttgcatcaagaactgccaccgtgtgaaaaccctgagcctgggtttctttcacaacagcccgaaggaagaggaagaggagcgtcgtggtggccgtccgctggaccaggtgcaatgcgtttttccggatacccatgtggcgtgcagcagccgtctggttaactgctgcctgaccagcagcttctgccgtggtctgtttagcagcctgagcaccaaccgtagcctgaccgaactggacctgagcgataacaccctgggtgatccgggcatgcgtgtgctgtgcgaggcgctgcagcacccgggttgcaacatccaacgtctgtggctgggtcgttgcggcctgagccaccagtgctgcttcgacattagcagcgtgctgagcagcagccaaaaactggttgagctggatctgagcgacaacgcgctgggtgattttggcatccgtctgctgtgcgtgggtctgaaacacctgctgtgcaacctgcagaagctgtggctggttagctgctgcctgaccagcgcgtgctgccaagacctggcgctggtgctgagcagcaaccacagcctgacccgtctgtacatcggcgaaaacgcgctgggtgacagcggcgtgcaggttctgtgcgagaagatgaaagatccgcagtgcaacctgcaaaaactgggtctggttaacagcggcctgaccagcatttgctgcagcgcgctgaccagcgtgctgaagaccaaccagaacttcacccacctgtatctgcgtagcaacgcgctgggtgataccggcctgcgtctgctgtgcgaaggtctgctgcacccggactgcaagctgcaaatgctggagctggataactgcagcctgaccagccacagctgctggaacctgagcaccatcctgacccacaaccacagcctgcgtaaactgaacctgggtaacaacgacctgggcgatctgtgcgtggttaccctgtgcgaggttctgaagcagcaaggttgcctgctgcagagcctgcaactgggcgagatgtacctgaaccgtgaaaccaaacgtgcgctggaggcgctgcaggaagagaagccggaactgaccatcgtgtttgagattagctggtaatgaaagctt

SEQ ID NO: 446 - protein, 107 aa, Artificial Sequence
  Description of Artificial Sequence Synthetic polypeptide
MHHHHHHDYKDDDDKKMASTRCKLARYLEDLEDVDLKKFKMHLEDYPPQKGCIPLPRGQTEKADHVDLATLMIDFNGEEKAWAMAVWIFAAINRRDLYEKAKRDEPK

SEQ ID NO: 447 - DNA, 333 bp, Artificial Sequence
  Description of Artificial Sequence Synthetic polynucleotide
atgcatcaccaccaccaccacgattacaaggatgatgatgataagaagatggcgagcacccgttgcaagctggcgcgttatctggaagacctggaggacgttgatctgaagaaattcaaaatgcacctggaagattacccgccgcagaagggttgcatcccgctgccgcgtggccaaaccgagaaagcggaccacgttgatctggcgaccctgatgattgacttcaacggcgaggaaaaggcgtgggcgatggcggtgtggatctttgcggcgattaaccgtcgtgatctgtatgaaaaggcgaaacgtgacgagccgaaataatgaaagctt

SEQ ID NO: 448 - protein, 105 aa, Artificial Sequence
  Description of Artificial Sequence Synthetic polypeptide
MHHHHHHDYKDDDDKTSVRCKLAQYLEDLEDVDLKKFKMHLEDYPPEKGCIPVPRGQMEKADHLDLATLMIDFNGEEKAWAMAVWIFAAINRRDLWEKAKKDQPE

SEQ ID NO: 449 - DNA, 327 bp, Artificial Sequence
  Description of Artificial Sequence Synthetic polynucleotide
atgcatcaccaccaccaccacgattacaaggatgatgatgataagacaagcgtgcgttgcaaactggcgcagtatctggaggacctggaagacgttgatctgaagaaattcaaaatgcacctggaggattacccgccggaaaagggttgcatcccggttccgcgtggccaaatggaaaaagcggaccacctggatctggcgaccctgatgattgacttcaacggcgaggaaaaggcgtgggcgatggcggtgtggatctttgcggcgattaaccgtcgtgatctgtgggaaaaggcgaagaaagaccagccggagtaatgaaagctt

SEQ ID NO: 450 - protein, 209 aa, Artificial Sequence
  Description of Artificial Sequence Synthetic polypeptide
MHHHHHHDYKDDDDKGRARDAILDALENLTAEELKKFKLKLLSVPLREGYGRIPRGALLSMDALDLTDKLVSFYLETYGAELTANVLRDMGLQEMAGQLQAATHQGSGAAPAGIQAPPQSAAKPGLHFIDQHRAALIARVTNVEWLLDALYGKVLTDEQYQAVRAEPTNPSKMRKLFSFTPAWNWTCKDLLLQALRESQSYLVEDLERS

SEQ ID NO: 451 - DNA, 639 bp, Artificial Sequence
  Description of Artificial Sequence Synthetic polynucleotide
atgcatcaccaccaccaccacgattacaaggatgatgatgataagggacgtgcgcgtgatgcgatcctggacgcgctggagaacctgaccgcggaggaactgaagaaattcaagctgaaactgctgagcgtgccgctgcgtgaaggttatggccgtattccgcgtggtgcgctgctgagcatggatgcgctggatctgaccgacaagctggtgagcttttacctggagacctatggcgcggaactgaccgcgaacgttctgcgtgatatgggtctgcaagagatggcgggtcagctgcaagcggcgacccaccaaggtagcggtgcggcgccggcgggtatccaagcgccgccgcaaagcgcggcgaagccgggtctgcacttcattgaccagcaccgtgcggcgctgattgcgcgtgtgaccaacgttgaatggctgctggatgcgctgtacggcaaagtgctgaccgacgagcagtatcaagcggttcgtgcggaaccgaccaacccgagcaagatgcgtaaactgttcagctttaccccggcgtggaactggacctgcaaagatctgctgctgcaggcgctgcgtgagagccaaagctacctggttgaggacctggaacgtagctaatgaaagctt

SEQ ID NO: 452 - protein, 210 aa, Artificial Sequence
  Description of Artificial Sequence Synthetic polypeptide
KLGSGSSGSGENLYFQGMGRARDAILDALENLSGDELKKFKMKLLTVQLREGYGRIPRGALLQMDAIDLTDKLVSYYLESYGLELTMTVLRDMGLQELAEQLQTTKEESGAVAAAASVPAQSTARTGHFVDQHRQALIARVTEVDGVLDALHGSVLTEGQYQAVRAETTSQDKMRKLFSFVPSWNLTCKDSLLQALKEIHPYLVMDLEQS

(19) United States
(12) Patent Application Publication
(10) Pub. No.: US 2023/0027739 A1
(43) Pub. Date: Jan. 26, 2023

(54) ANTIGEN-BINDING AND ANTIGEN DEGRADATION CONSTRUCTS

(71) Applicant: Entrada Therapeutics, Inc., Boston, MA (US)

(72) Inventors: Natarajan SETHURAMAN, Boston, MA (US); Patrick DOUGHERTY, Boston, MA (US); Byung-Kwon CHOI, Boston, MA (US); Nicholas Emerson LONG, Sunapee, NH (US); Ziqing QIAN, Boston, MA (US)

(21) Appl. No.: 17/739,943

(22) Filed: May 9, 2022

Related U.S. Application Data
(60) Provisional application No. 63/186,664, filed on May 10, 2021.
(60) Provisional application No. 63/216,983, filed on Jun. 30, 2021.
(60) Provisional application No. 63/239,671, filed on Sep. 1, 2021.
(60) Provisional application No. 63/362,295, filed on Mar. 31, 2022.
(60) Provisional application No. 63/290,960, filed on Dec. 17, 2021.
(60) Provisional application No. 63/298,565, filed on Jan. 11, 2022.
(60) Provisional application No. 63/268,577, filed on Feb. 25, 2022.

(51) Int. Cl. C07K 7/64; C07K 16/10; C07K 16/28; C07K 16/40; C07K 16/18; C12N 9/10

(52) U.S. Cl. CPC C07K 7/64; C07K 16/1036; C07K 16/28; C07K 16/40; C07K 16/18; C12Y 203/02; C12N 9/104; C07K 2319/10; C07K 2317/569; C07K 2317/90; C07K 2319/30

(57) ABSTRACT

Degradation compounds include a cyclic cell penetrating peptide (cCPP) and a degradation construct. The degradation construct includes a degradation moiety and a targeting moiety. The targeting moiety binds a target protein. When the targeting moiety is bound to the target protein, the degradation moiety mediates degradation of the target protein. The cCPP facilitates transfer of the degradation construct into a cell. The degradation compound may further include an exocyclic peptide to enhance endosomal escape of the compound or degradation construct once inside the cell.

### CROSS REFERENCE TO RELATED APPLICATIONS

This application claims the benefit of U.S. Provisional Application Ser. No. 63/186,664, filed on May 10, 2021; 63/216,983, filed Jun. 30, 2022; 63/362,295, filed on Mar. 31, 2022; 63/239,671, filed on Sep. 1, 2021; and 63/290,960 filed on Dec. 17, 2021; 63/298,565, filed on Jan. 11, 2022; and 63/268,577, filed on Feb. 25, 2022, which are incorporated by reference herein in their respective entireties.

### SEQUENCE LISTING

The instant application contains a Sequence Listing which has been submitted electronically in ASCII format and is hereby incorporated by reference in its entirety. Said ASCII copy, created on Jul. 15, 2022, is named 0655_000029US01_SL.txt and is 604,384 bytes in size.

### FIELD OF THE INVENTION

Provided herein are compositions and methods for facilitating degradation of a target protein.

### BACKGROUND

Disease, such as cancer, degenerative diseases, and genetic diseases, can be caused by mutations in intracellular proteins. The effectiveness of a therapeutic agent to treat such diseases is often dependent on its ability to penetrate cellular membranes in order to access an intracellular target and/or induce a desired change in biological activity. Although many therapeutic candidates show promising biological activity in vitro, many fail to reach or penetrate target cells to achieve the desired effect in vivo, often due to physiochemical properties that result in inadequate biodistribution. Adequate delivery into a cell or cellular compartment of interest is a particularly acute problem for larger molecules, such as proteins, antibodies and antibody-like moieties.

Selective degradation of intracellular proteins is a promising potential alternative to protein inhibition for therapeutic intervention. Degradation of target intracellular proteins offers important advantages over small molecule inhibitors. While small molecule inhibitors act via target occupancy, degradation offers a long-lasting effect by eliminating the target until re-synthesis, which can take hours or days. Moreover, by triggering the degradation of the protein target, all functional sites are removed, abolishing all target protein functions. Such ‘knockdown’ is not feasible with small molecule inhibitor drugs, which typically interact with only one site on the target, leaving the others to function normally. In addition, many intracellular target proteins of clinical significance are currently classed as “poorly druggable”—meaning that small molecule inhibitor drugs are unlikely to be effective.

Further, conventional therapeutic strategies, which rely on small molecules to inhibit the activity of target proteins, are not able to address the underlying cause of these diseases (e.g., protein mutations that result in improper folding or aberrant activity). Moreover, small molecules generally are not able to inhibit the activity of these intracellular proteins. The present disclosure addresses these needs.

### SUMMARY

The present disclosure describes, among other things, compounds for degradation of target proteins. In embodiments, the target proteins are intracellular proteins.

The compounds, also referred to herein as “degradation compounds,” include a degradation construct and a cell penetrating peptide (CPP). The CPP facilitates intracellular delivery of the degradation construct. In embodiments, the CPP is a cyclic CPP (cCPP). In embodiments, the compounds further include an exocyclic peptide (EP). In embodiments, the EP enhances endosomal escape and allows the degradation construct to enter the cytosol to interact with, and facilitate degradation of, the target protein.

Moieties that facilitate intracellular delivery through endocytosis and that facilitate endosomal escape are referred to herein as endosomal escape vehicles (EEVs). EEVs as described herein comprise a cCPP. In embodiments, the EEV comprises an EP.

In embodiments, the degradation construct facilitates proteasomal or autophagy driven degradation of the target protein. In embodiments, the target protein is an intracellular protein, a cell membrane protein, or the like.

In embodiments, the degradation construct includes a targeting moiety and a degradation moiety. The targeting moiety binds to the target protein. In embodiments, the targeting moiety is an antibody, an antigen binding fragment, or an antibody mimetic. The degradation moiety mediates degradation of the target protein and/or is capable of recruiting an endogenous enzyme capable of mediating the degradation of the target protein. In embodiments, the degradation moiety mediates the degradation of the target protein when the targeting moiety is bound to the target protein. In embodiments, the degradation moiety recruits an endogenous enzyme capable of mediating the degradation of the target protein when the targeting moiety is bound to the target protein.

In embodiments, the cCPP is of Formula (A):

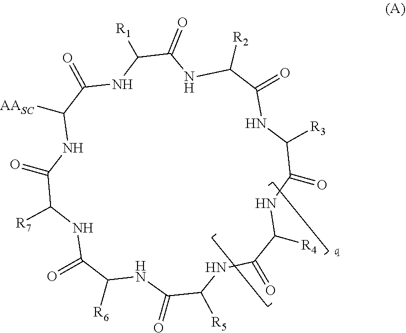

or a protonated form thereof, wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;
AA_{SC} is an amino acid side chain; and
q is 1, 2, 3 or 4.

In embodiments, the cCPP is of Formula (A) is of Formula (I):

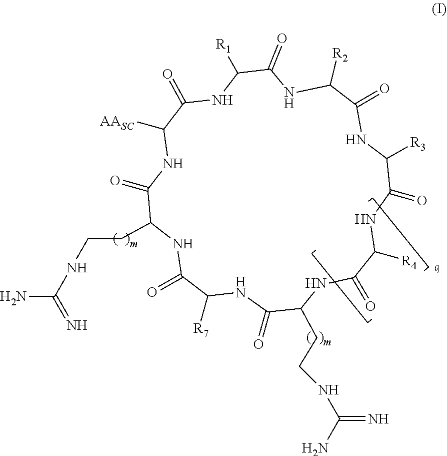

or a protonated form or salt thereof,

wherein each m is independently an integer from 0-3.

In embodiments, the cCPP is of Formula (A) is of Formula (I-1):

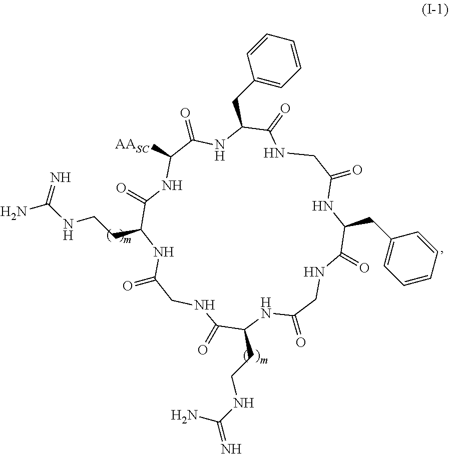

or a protonated form or salt thereof.

In embodiments, the cCPP is of Formula (A) is of Formula (I-2):

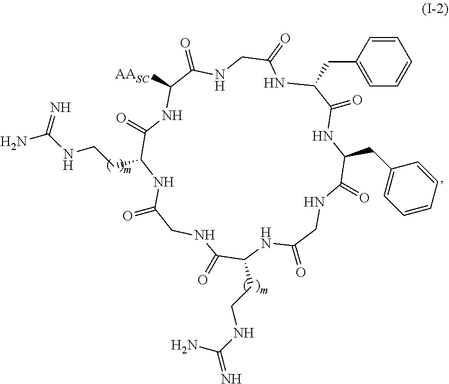

or a protonated form or salt thereof.

In embodiments, the cCPP is of Formula (A) is of Formula (I-3):

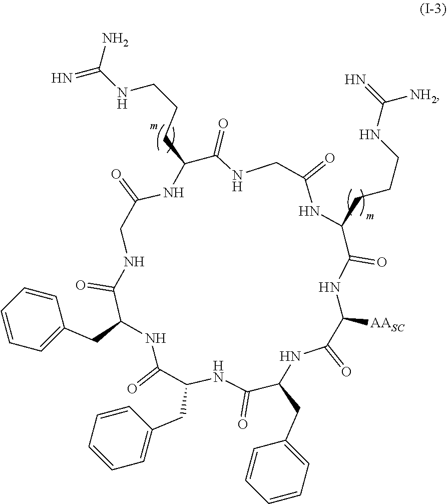

or a protonated form or salt thereof.

In embodiments, the cCPP is of Formula (A) is of Formula (I-4):

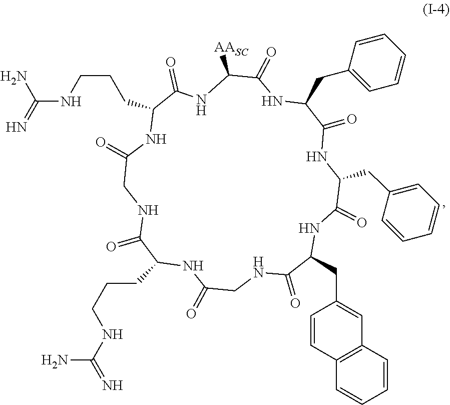

or a protonated form or salt thereof.

In embodiments, the cCPP is of Formula (A) is of Formula (I-5):

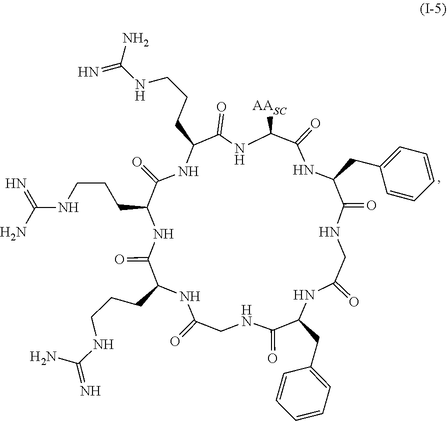

or a protonated form or salt thereof.

In embodiments the cCPP is of Formula (A) is of Formula (I-6):

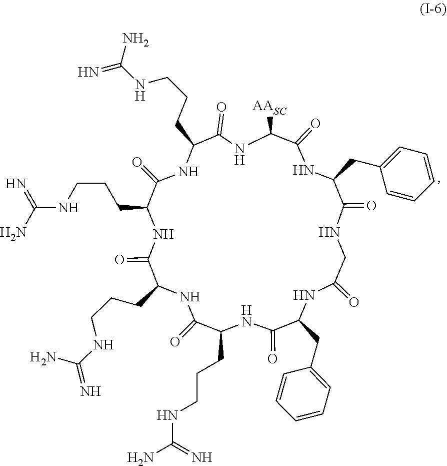

or a protonated form or salt thereof.

In embodiments, the cCPP is of Formula (II):

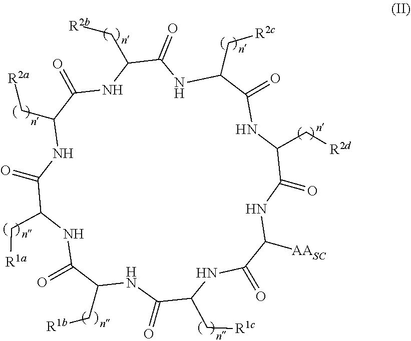

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain; at least one of R^{2a}, R^{2b}, R^{2c} and R^{2d} is

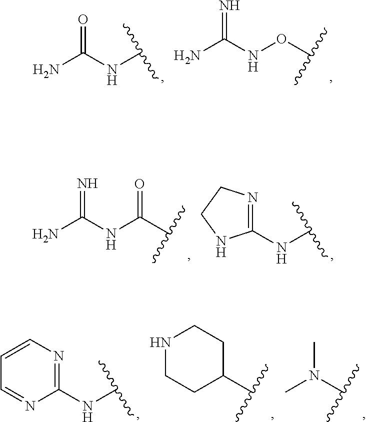

or a protonated form or salt thereof;
at least one of R^{2a}, R^{2b}, R^{2c} and R^{2d} is guanidine or a protonated form or salt thereof,
each n″ is independently an integer from 0 to 5;
each n′ is independently an integer from 0 to 3; and
if n′ is 0 then R^{2a}, R^{2b}, R^{2c} or R^{2d} is absent.

In embodiments, the cCPP of Formula (II) is of Formula (II-1):

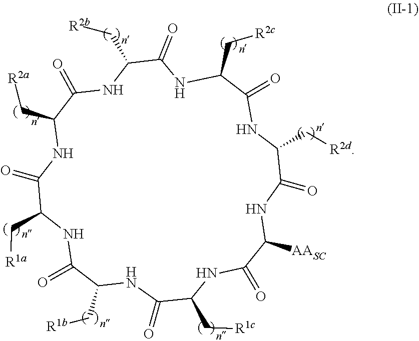

In embodiments, the cCPP of Formula (II) is of Formula (IIa):

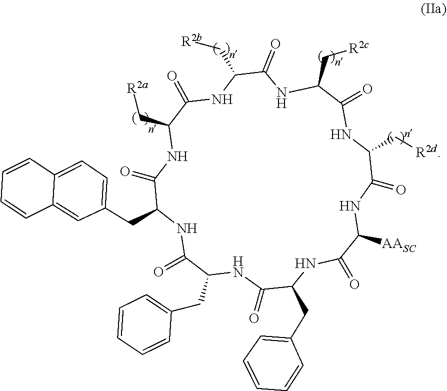

In embodiments, the cCPP of Formula (II) is of Formula (IIb):

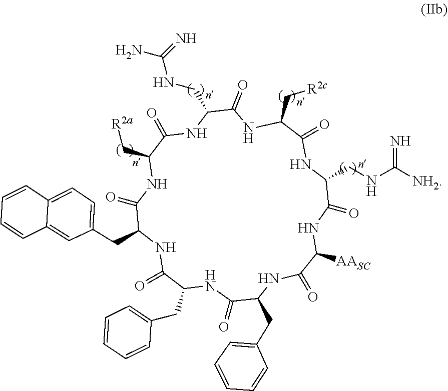

In embodiments, the cCPP of Formula (II) is of Formula (IIc):

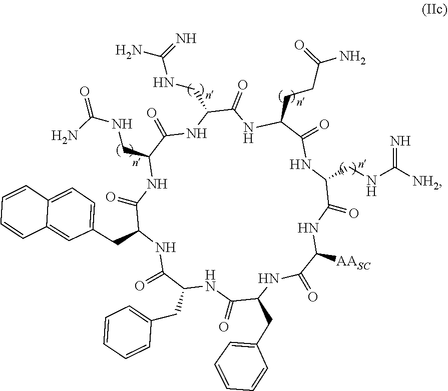

or a protonated form or salt thereof.

In embodiments, the cCPP has the structure:

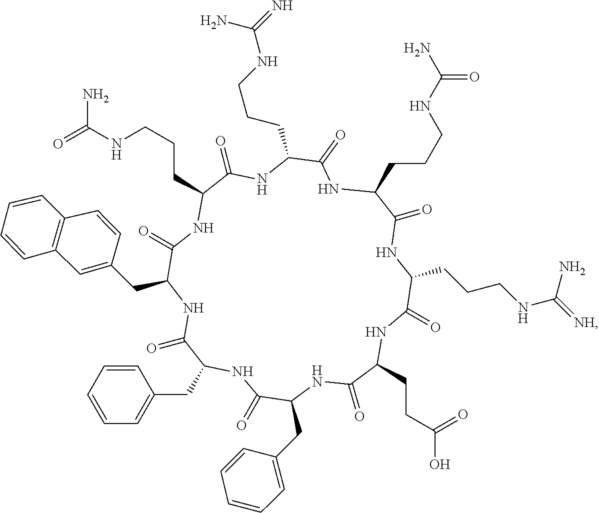 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

or a protonated form or salt thereof, wherein at least one atom of an amino acid side chain is replaced by the degradation compound or a linker or at least one lone pair forms a bond to the degradation compound or the linker.

In embodiments, the cCPP has the structure:

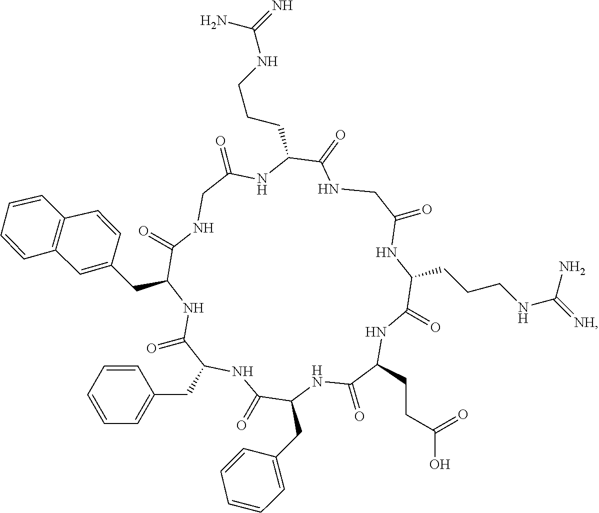 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

or a protonated form or salt thereof, wherein at least one atom of an amino acid side chain is replaced by the degradation compound or a linker or at least one lone pair forms a bond to the degradation compound or the linker.

In embodiments, the compound comprises an exocyclic peptide (EP). In embodiments, the EP comprises one of the following sequences: KK, KR, RR, HH, HK, HR, RH, KKK, KGK, KBK, KBR, KRK, KRR, RKK, RRR, KKH, KHK, HKK, HRR, HRH, HHR, HBH, HHH, HHHH (SEQ ID NO: 1), KHKK (SEQ ID NO: 2), KKHK (SEQ ID NO: 3), KKKH (SEQ ID NO: 4), KHKH (SEQ ID NO: 5), HKHK (SEQ ID NO: 6), KKKK (SEQ ID NO: 7), KKRK (SEQ ID NO: 8), KRKK (SEQ ID NO: 9), KRRK (SEQ ID NO: 10), RKKR (SEQ ID NO: 11), RRRR (SEQ ID NO: 12), KGKK (SEQ ID NO: 13), KKGK (SEQ ID NO: 14), HBHBH (SEQ ID NO: 15), HBKBH (SEQ ID NO: 16), RRRRR (SEQ ID NO: 17), KKKKK (SEQ ID NO: 18), KKKRK (SEQ ID NO: 19), RKKKK (SEQ ID NO: 20), KRKKK (SEQ ID NO: 21), KKRKK (SEQ ID NO: 22), KKKKR (SEQ ID NO: 23), KBKBK (SEQ ID NO: 24), RKKKKG (SEQ ID NO: 25), KRKKKG (SEQ ID NO: 26), KKRKKG (SEQ ID NO: 27), KKKKRG (SEQ ID NO: 28), RKKKKB (SEQ ID NO: 29), KRKKKB (SEQ ID NO: 30), KKRKKB (SEQ ID NO: 31), KKKKRB (SEQ ID NO: 32), KKKRKV (SEQ ID NO: 33), RRRRRR (SEQ ID NO: 34), HHHHHH (SEQ ID NO: 35), RHRHRH (SEQ ID NO: 36), HRHRHR (SEQ ID NO: 37), KRKRKR (SEQ ID NO: 38), RKRKRK (SEQ ID NO: 39), RBRBRB (SEQ ID NO: 40), KBKBKB (SEQ ID NO: 41), PKKKRKV (SEQ ID NO: 42), PGKKRKV (SEQ ID NO: 43), PKGKRKV (SEQ ID NO: 44), PKKGRKV (SEQ ID NO: 45), PKKKGKV (SEQ ID NO: 46), PKKKRGV (SEQ ID NO: 47) or PKKKRKG (SEQ ID NO: 48), wherein B is beta-alanine.

In embodiments, the compound is of Formula (C):

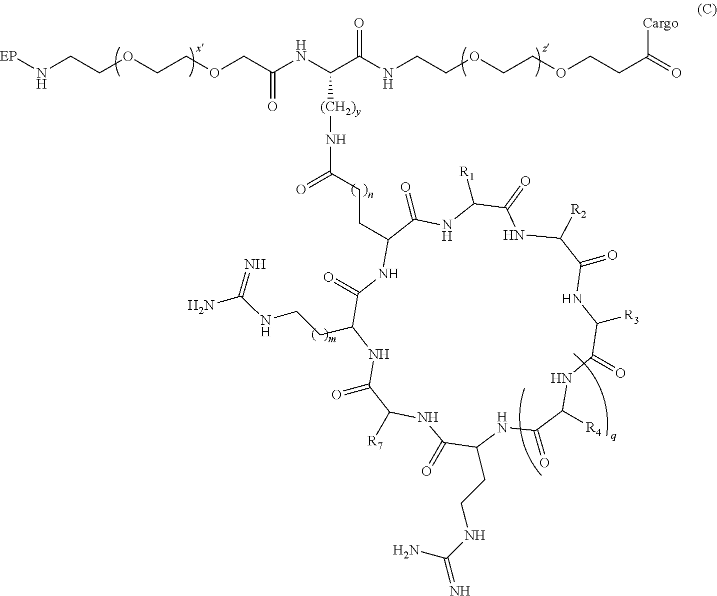

or a protonated form or salt thereof, wherein.
R₁, R₂, and R₃ are each independently H or a side chain comprising an aryl or heteroaryl group, wherein at least one of R₁, R₂, and R₃ is a side chain comprising an aryl or heteroaryl group;
R₄ and R₇ are independently H or an amino acid side chain;
EP is an exocyclic peptide;
each m is independently an integer from 0-3;
n is an integer from 0-2;
x′ is an integer from 1-23;
y is an integer from 1-5;
q is an integer from 1-4;
z′ is an integer from 1-23, and
Cargo is the degradation compound.

In embodiments, targeting moiety specifically binds β-catenin, NALP3, KRAS, MDM2, EGFR, ASC, or IRF-5.

### BRIEF DESCRIPTION OF THE DRAWINGS

FIG. 1 is a schematic representation of a degradation construct and mechanisms of action of a degradation construct.

FIGS. 2A-B are schematic drawings showing examples of an IgG1 antibody (A) and camelid single chain antibody (B) and antigen binding fragments thereof.

FIGS. 3A-B show SDS-PAGE and western blot analysis showing the expression and purification of K19-Fc-Cys (A) and K19-Fc(S239C) (B) from mammalian cells.

FIG. 4 shows an SDS-PAGE analysis showing the expression of bispecific 7D12-K19 construct expression in P. pastoris and their culture supernatants.

FIGS. 5A-B show an SDS-PAGE analysis of unconjugated K19-Cys and conjugated K19-Cys-EEV12-PEG8 (A) and the structure of EEV12-PEG8-K(mal)(B).

FIG. 6 is a plot showing the effect of K19-Cys and K19-Cys-EEV12 on the viability of various human cancer cell lines after treatment of 72 h.

FIGS. 7A-C are plots showing the effect of K19-Cys or K19-Cys-EEV12 on the viability of LS180 (A), SW480 (B), and HCT116 (C) cell lines after treatment of 72 h.

FIGS. 8A-B are plots showing the effect of K19-Cys and K19-Cys-EEV12 on the phosphorylation of ERK1/2 of HCT116 cell line (A) and on the phosphorylation of MEK and ERK1/2 of SW480 cell line (B) after treatment of 24 h.

FIGS. 9A-8D show SDS-PAGE analysis showing the expression of BC2-Cys (A), BC2-Fc-cys (B), cys-BC2-VHLpep (C), and BC2-Mb-cys (D) from P. pastoris.

FIG. 10 is an SDS-PAGE gel showing the conjugation of BC2-Mb-Cys with EEV12-PEG8-K(mal).

FIGS. 11A-C are plots showing the effects of BC2-Fc-Cys and BC2-Fc-Cys-EEV12 on β-catenin and c-Myc levels in HCT-116 cells (A), and the effects of BC2-Mb-Cys and BC2-Mb-Cys-EEV12 on β-catenin and c-Myc levels in HCT-116 cells (B) and SNU-398 (C) cells after 24 h treatments.

FIGS. 12A-C are plots showing the effects of BC2-Fc-Cys, BC2-Fc-Cys-EEV12, BC2-Mb-Cys, and BC2-Mb-Cys-EEV12 on cell viability of HCT-116 (A and B) and SNU-398 cells (C) after 120 h or 72 h treatments.

FIG. 13 shows SDS-PAGE gels showing the conjugation of MOP3+ with EEV12-PEG4 and EEV12-PEG8.

FIGS. 14A-B are plots showing the relative binding affinity of MOP3 (A), MOP3+ (A), and MOP3+-EEV12 (B) to GST-MDM2 or GST-MDM4 as determined by competitive fluorescence polarization.

FIG. 15 is a plot illustrating the anti-proliferative effects of compounds at the indicated concentrations on the viability of p53-wild type (SJSA-1) and p53-mutant (SW480) cell lines after 72 h of treatment.

FIG. 16 shows SDS-PAGE analysis demonstrating the EEV conjugation of a mouse IgG AG-20B under reducing and non-reducings conditions of unconjugated. The structure of EEV12-PEG8-TFP is also shown.

FIGS. 17A-B show western blot analysis demonstrating the EEV12-assisted anti-NLRP3 IgG uptake in human monocytes derived macrophages (A) an THP1 derived macrophages (B).

FIG. 18 shows SDS-PAGE and western blot analysis of the expression and purification of a VHH^{CARD} with a C-terminal cysteine from E. coli.

FIG. 19 shows SDS-PAGE and western blot analysis of the expression and purification of a VHH^{CARD}-Fc fusion from mammalian cells.

FIG. 20 shows SDS-PAGE and western blot analysis of the expression and purification of a VHH^{CARD}-Mb from mammalian cells.

FIG. 21 shows SDS-PAGE analysis of the expression of VHH^{CARD}-Cys, VHH^{CARD}-Fc-Cys, VHH^{CARD}-Mb-Cys, Cys-VHH^{CARD}-VHL₁₅₂₋₂₁₃ from P. pastoris.

FIG. 22 shows SDS-PAGE analysis demonstrating the bioconjugation of VHH^{CARD}-Fc fusion with EEV12 under reducing and non-reducing PAGE conditions. Lane 2 is the unconjugated VHH^{CARD}-Fc; lane 3 is the conjugated products of VHH^{CARD}-Fc-EEV12.

FIG. 23 shows western blot analysis demonstrating the EEV12-assisted uptake of VHH^{CARD}-Fc in human monocytes derived macrophages and THP-1 derived macrophages.

### DETAILED DESCRIPTION

### Overview

The present disclosure describes, among other things, degradation compounds for degradation of target proteins. In embodiments, the target proteins are intracellular proteins. The degradation compounds include a degradation construct and a cell penetrating peptide (CPP). In embodiments, the degradation construct comprises a targeting moiety and a degradation moiety.

The targeting moiety binds the target protein. The degradation moiety mediates degradation of the target protein and/or is capable of recruiting an endogenous enzyme capable of mediating the degradation of the target protein.

The CPP facilitates intracellular delivery of the degradation construct. In embodiments, the CPP is a cyclic CPP (cCPP). In embodiments, the compounds further include an exocyclic peptide (EP). In embodiments, the EP enhances endosomal escape and allows the degradation construct to enter the cytosol to interact with, and facilitate degradation of, the target protein.

### Degradation Compounds

The present disclosure provides degradation compounds. The degradation compounds facilitate degradation of a target protein. As used herein, “target protein” refers to a protein that is targeted for degradation. In embodiments, the degradation compound facilitates proteasomal degradation of a target protein. In embodiments, the degradation compound facilitates autophagy mediated degradation of a target protein. In embodiments, the degradation compound regulates the levels and/or activity of a target protein within a cell. In embodiments, the degradation compound decreases the level of the target protein. In embodiments, the degradation compound increases or decreases the level and/or activity of a protein, transcript, or gene that is regulated by the target protein.

The degradation compounds include a degradation construct and a cyclic cell penetrating peptide (cCPP). In embodiments, the cCPP is, or forms a part of, an endosomal escape vehicle (EEV). The degradation construct includes a targeting moiety and a degradation moiety.

Mechanisms of Degradation

To maintain homeostasis, cells regulate the degradation of proteins that are misfolded, have aberrant activity, are mutated, or have become obsolete in the cells current phenotype. Autophagy mediated degradation and proteasomal degradation are the two main mechanism by which cells can degrade proteins.

### Autophagy Degradation Mechanism

Autophagy is the removal of proteins and/or organelles through a lysosome-mediated process. There are several types of autophagy including macroautophagy, microautophagy, chaperone-mediated autophagy, mitophagy, and lipophagy. Macroautophagy is the primary autophagy process used to remove cytolytic proteins. In macroautophagy a double membrane autophagosome engulfs the target protein. The autophagosome fuses with a lysosome or vacuole.

### Proteasomal Degradation Mechanism

Proteins may be degraded by the proteasome, a protein complex of various proteases that degrade proteins via proteolysis. Degradation via the proteasome is termed proteasomal degradation. Proteins that have polyubiquitin tags may be targeted for proteasomal degradation.

The process of adding a polyubiquitin tag involves several proteins and enzymatic reactions. In embodiments, degradation compounds and degradation constructs are provided that mediate the addition of a polyubiquitin tag on a target protein for proteasomal degradation of the target protein.

Ubiquitin is a 76 amino acid protein that may be conjugated to proteins as a post translational modification to proteins. The process of adding ubiquitin to a target protein is termed ubiquitylation (also known as ubiquitination or ubiquitinylation). Polyubiquitylated proteins may be targeted for proteasomal degradation. A first ubiquitin is conjugated to a target protein through a covalent bond between the C-terminal carboxylate of the ubiquitin and a lysine, cysteine, serine, or threonine side chain or the N-terminus of the target protein. A second ubiquitin can be conjugated to the first ubiquitin through a covalent bond between the C-terminal carboxylate of the second ubiquitin and a lysine or methionine side chain on the first ubiquitin. The nature of the ubiquitin linkages in a polyubiquitin chain specifies the fate of the target protein. For example, chains of four or more ubiquitin molecules linked through K48 and chains of ubiquitin linked through K11 often signal for proteasomal degradation of the protein to which they are conjugated.

The ubiquitylation process involves three enzymes; a ubiquitin-activating enzymes, ubiquitin-conjugating enzyme, and a ubiquitin ligase colloquially termed E1, E2, and E3 respectively. E1 activates ubiquitin by catalyzing an ATP dependent reaction resulting in a thioester linkage between the C-terminus of ubiquitin and a cysteine within the active site of E1. E2 catalyzes the transfer of the activated ubiquitin to a cysteine within the active site of E2 via a transthioesterification reaction. E3 catalyzes the transfer of ubiquitin from E2 to the target protein.

There are two known human E1 enzymes, 35 known E2 enzymes, and over 600 known E3 ligases. The large number of E1, E2, and E3 proteins allows for vast diversity and specificity in the ubiquitylation process. The E1 enzymes include UBA1 and UBA6. The E2 enzymes include, but are not limited to, Ube2A, Ube2B, Ube2B, Ube2D1, Ube2D2, Ube2D3, Ube2D4, Ube2E1, Ube2E2, Ube2E3, Ube2G1, Ube2G2, Ube2H, Ube2J1, Ube2J2, Ube2K, Ube2L3, Ube2N, Ube2NL, Ube2O, Ube2Q1, Ube2Q2, Ube2QL, Ube2R1, Ube2R2, Ube2S, Ube2T, Ube2V1, Ube2V2, Ube2W, BIRC6, Ube2F, Ube2I, Ube2L6, Ube2M, Ube2Z, ATG10, and ATG3.

The E3 ligases can be classified in several categories including the homologous to E6-associated protein C-terminus (HECT) domain ligases, the Really Interesting New Gene (RING) domain ligases, and the U-box ubiquitin family of ligases (UUL). RING and UUL E3 ligases catalyze the direct transfer of ubiquitin to the target protein. In contrast, HECT E3 ligases require an intermediate step. HECT E3 ligases first catalyze the transfer of the ubiquitin form the E2 to an active cysteine on the HECT E3 ligase before catalyzing the transfer of the ubiquitin from the HECT E3 ligase to the target protein. UULs are a family of modified RING E3 ligases the do not have the full complement of Zn²⁺ binding ligands. While HECT E3 ligases have a direct role in catalysis during ubiquitination, RING and U-box E3 proteins facilitate protein ubiquitination by acting as adaptor molecules that recruit E2 and substrate molecules to promote substrate ubiquitination. Although many RING-type E3 ligases, such as MDM2 (murine double minute clone 2 oncoprotein) and c-Cbl, may act alone, others are found as components of much larger multi-protein complexes, such as the anaphase-promoting complex (“APC”). Table 1A lists some examples of E3 ligases.

Some E3 ligases are E3 ligase complexes that accessory proteins in addition to the protein that is directly involved in catalyzing ubiquitination of the target protein. For example, cullin-RING ligases (CRL) are E3 ligase complexes that catalyze ubiquitinylation of a target protein (Mahon et al., Biomolecules (2014), 13, 4(4):897-930; Jackson et al., Trends Ciochem Sci. (2009), 34(11): 562-570). CRLs include a cullin scaffold protein that recruits a RBX1 or RBX2 (E3 ligases). The cullin scaffold also binds to an adaptor protein. The adaptor protein binds to the target protein. In some cases, the adaptor protein binds to a substrate receptor protein, the substrate receptor protein binds to the target protein. There are seven cullin proteins (Cul1, Cul2, Cul3, Cul4a, Cul4b, Cul5, and Cul7. There are many adaptor proteins including, but not limited to, SKP1, elongin B/C heterodimer, and DDB1. Additionally, there are many substrate receptor proteins including but not limited to, FBP, various SOCS proteins, and various DCAF proteins.

An example of a CRL is the anaphase-promoting complex. Another example of a CLR is the SKP, Cullin, F-box containing complex (SCF). Table 3 gives examples of accessory proteins involved in E3 ligase complexes. SCF complexes include CUL1 as a scaffold protein, RBX1 as the RING ligase, SKP1 as an adaptor protein, and an F-box containing protein. Table T2 includes examples of E3 ligase accessory proteins. Table 1B lists some examples of accessory proteins involved in E3 ligase complexes.

TABLE 1A
Example E3 ligases and classes thereof

| E3 ligase | Class |
|---|---|
| AFF4 | UBOX |
| AMFR | RING |
| ANAPC11 | RING |
| ANKIB1 | RING |
| AREL1 | HECTc |
| ARIH1 | RING |
| ARIH2 | RING |
| BARD1 | RING |
| BFAR | RING |
| BIRC2 | RING |
| BIRC3 | RING |
| BIRC7 | RING |
| BIRC8 | RING |
| BMI1 | RING |
| BRAP | RING |
| BRCA1 | RING |
| CBL | RING |
| CBLB | RING |
| CBLC | RING |
| CBLL1 | RING |
| CCDC36 | RING |
| CCNB1IP1 | RING |
| CGRRF1 | RING |
| CHFR | RING |
| CNOT4 | RING |
| CUL9 | RING |
| CYHR1 | RING |
| DCST1 | RING |
| DTX1 | RING |
| DTX2 | RING |
| DTX3 | RING |
| DTX3L | RING |
| DTX4 | RING |
| DZIP3 | RING |
| E4F1 | zf-C2H2 |
| FANCL | RING |
| G2E3 | HECTc |
| HACE1 | HECTc |
| HECTD1 | HECTc |
| HECTD2 | HECTc |
| HECTD3 | HECT |
| HECTD4 | HECTc |
| HECW1 | HECTc |
| HECW2 | HECTc |
| HERC1 | HECTc |
| HERC2 | HECTc |
| HERC3 | HECTc |
| HERC4 | HECTc |
| HERC5 | HECTc |
| HERC6 | HECTc |
| HLTF | RING |
| HUWE1 | HECTc |
| IRF2BP1 | RING |
| IRF2BP2 | RING |
| IRF2BPL | RING |
| Itch | HECTc |
| KCMF1 | RING |
| KMT2C | RING |
| KMT2D | RING |
| LNX1 | RING |
| LNX2 | RING |
| LONRF1 | RING |
| LONRF2 | RING |
| LONRF3 | RING |
| LRSAM1 | RING |
| LTN1 | RING |
| MAEA | RING |
| MAP3K1 | RING |
| MARCH1 | RING |
| MARCH10 | RING |
| MARCH11 | RING |
| MARCH2 | RING |
| MARCH3 | RING |
| MARCH4 | RING |
| MARCH5 | RING |
| MARCH6 | RING |
| MARCH7 | RING |
| MARCH8 | RING |
| MARCH9 | RING |
| Mdm2 | RING |
| MDM4 | RING |
| MECOM | RING |
| MEX3A | RING |
| MEX3B | RING |
| MEX3C | RING |
| MEX3D | RING |
| MGRN1 | RING |
| MIB1 | RING |
| MIB2 | RING |
| MID1 | RING |
| MID2 | RING |
| MKRN1 | RING |
| MKRN2 | RING |
| MKRN3 | RING |
| MKRN4P | RING |
| MNAT1 | RING |
| MSL2 | RING |
| MUL1 | RING |
| MYCBP2 | RING |
| MYLIP | RING |
| NEDD4 | HECTc |
| NEDD4L | HECTc |
| NEURL1 | RING |
| NEURL1B | RING |
| NEURL3 | RING |
| NFX1 | RING |
| NFXL1 | RING |
| NHLRC1 | RING |
| NOSIP | UBOX |
| NSMCE1 | RING |
| PARK2 | RING |
| PCGF1 | RING |
| PCGF2 | RING |
| PCGF3 | RING |
| PCGF5 | RING |
| PCGF6 | RING |
| PDZRN3 | RING |
| PDZRN4 | RING |
| PELI1 | PELI |
| PELI2 | PELI |
| PELI3 | PELI |
| PEX10 | RING |
| PEX12 | RING |
| PEX2 | RING |
| PHF7 | RING |
| Zswim2 | RING |
| CBX4 | — |
| PIAS1 | — |
| PIAS4 | — |
| PUB19 | UBOX |
| RBX2 | RING |
| PHRF1 | RING |
| PJA1 | RING |
| PJA2 | RING |
| PLAG1 | RING |
| PLAGL1 | RING |
| PML | RING |
| PPIL2 | UBOX |
| PRPF19 | UBOX |
| RAD18 | RING |
| RAG1 | RING |
| RAPSN | RING |
| RBBP6 | RING |
| RBCK1 | RING |
| RBX1 | RING |
| RC3H1 | RING |
| RC3H2 | RING |
| RCHY1 | RING |
| RFFL | RING |
| RFPL1 | RING |
| RFPL2 | RING |
| RFPL3 | RING |
| RFPL4A | RING |
| RFPL4AL1 | RING |
| RFPL4B | RING |
| RFWD2 | RING |
| RFWD3 | RING |
| RING1 | RING |
| RLF | RING |
| RLIM | RING |
| RMND5A | RING |
| RMND5B | RING |
| RNF10 | RING |
| RNF103 | RING |
| RNF11 | RING |
| RNF111 | RING |
| RNF112 | RING |
| RNF113A | RING |
| RNF113B | RING |
| RNF114 | RING |
| RNF115 | RING |
| RNF121 | RING |
| RNF122 | RING |
| RNF123 | RING |
| RNF125 | RING |
| RNF126 | RING |
| RNF128 | RING |
| RNF13 | RING |
| RNF130 | RING |
| RNF133 | RING |
| RNF135 | RING |
| RNF138 | RING |
| RNF139 | RING |
| RNF14 | RING |
| RNF141 | RING |
| RNF144A | RING |
| RNF144B | RING |
| RNF145 | RING |
| RNF146 | RING |
| RNF148 | RING |
| RNF149 | RING |
| RNF150 | RING |
| RNF151 | RING |
| RNF152 | RING |
| RNF157 | RING |
| RNF165 | RING |
| RNF166 | RING |
| RNF167 | RING |
| RNF168 | RING |
| RNF169 | RING |
| RNF17 | RING |
| RNF170 | RING |
| RNF175 | RING |
| RNF180 | RING |
| RNF181 | RING |
| RNF182 | RING |
| RNF183 | RING |
| RNF185 | RING |
| RNF186 | RING |
| RNF187 | RING |
| RNF19A | RING |
| RNF19B | RING |
| RNF2 | RING |
| RNF20 | RING |
| RNF207 | RING |
| RNF208 | RING |
| RNF212 | RING |
| RNF212B | RING |
| RNF213 | RING |
| RNF214 | RING |
| RNF215 | RING |
| RNF216 | RING |
| RNF217 | RING |
| RNF219 | RING |
| RNF220 | RING |
| RNF222 | RING |
| RNF223 | RING |
| RNF224 | RING |
| RNF225 | RING |
| RNF24 | RING |
| RNF25 | RING |
| RNF26 | RING |
| RNF31 | RING |
| RNF32 | RING |
| RNF34 | RING |
| RNF38 | RING |
| RNF39 | RING |
| RNF4 | RING |
| RNF40 | RING |
| RNF41 | RING |
| RNF43 | RING |
| RNF44 | RING |
| RNF5 | RING |
| RNF6 | RING |
| RNF7 | RING |
| RNF8 | RING |
| RNFT1 | RING |
| RNFT2 | RING |
| RSPRY1 | RING |
| SCAF11 | RING |
| SH3RF1 | RING |
| SH3RF2 | RING |
| SH3RF3 | RING |
| SHPRH | RING |
| SIAH1 | RING |
| SIAH2 | RING |
| ZXDC | RING |
| HUWE2 | HECTc |
| PIAS2 | — |
| RANBP2 | — |
| SIAH3 | RING |
| SMURF1 | HECTc |
| SMURF2 | HECTc |
| STUB1 | UBOX |
| SYVN1 | RING |
| TMEM129 | RING |
| Topors | RING |
| TRAF2 | RING |
| TRAF3 | RING |
| TRAF4 | RING |
| TRAF5 | RING |
| TRAF6 | RING |
| TRAF7 | RING |
| TRAIP | RING |
| TRIM10 | RING |
| TRIM11 | RING |
| TRIM13 | RING |
| TRIM15 | RING |
| TRIM17 | RING |
| TRIM2 | RING |
| TRIM21 | RING |
| TRIM22 | RING |
| TRIM23 | RING |
| TRIM24 | RING |
| TRIM25 | RING |
| TRIM26 | RING |
| TRIM27 | RING |
| TRIM28 | RING |
| TRIM3 | RING |
| TRIM31 | RING |
| TRIM32 | RING |
| TRIM33 | RING |
| TRIM34 | RING |
| TRIM35 | RING |
| TRIM36 | RING |
| TRIM37 | RING |
| TRIM38 | RING |
| TRIM39 | RING |
| TRIM4 | RING |
| TRIM40 | RING |
| TRIM41 | RING |
| TRIM42 | RING |
| TRIM43 | RING |
| TRIM43B | RING |
| TRIM45 | RING |
| TRIM46 | RING |
| TRIM47 | RING |
| TRIM48 | RING |
| TRIM49 | RING |
| TRIM49B | RING |
| TRIM49C | RING |
| TRIM49D1 | RING |
| TRIM5 | RING |
| TRIM50 | RING |
| TRIM51 | RING |
| TRIM52 | RING |
| TRIM54 | RING |
| TRIM55 | RING |
| TRIM56 | RING |
| TRIM58 | RING |
| TRIM59 | RING |
| TRIM6 | RING |
| TRIM60 | RING |
| TRIM61 | RING |
| TRIM62 | RING |
| TRIM63 | RING |
| TRIM64 | RING |
| TRIM64B | RING |
| TRIM64C | RING |
| TRIM65 | RING |
| TRIM67 | RING |
| TRIM68 | RING |
| TRIM69 | RING |
| TRIM7 | RING |
| TRIM71 | RING |
| TRIM72 | RING |
| TRIM73 | RING |
| TRIM74 | RING |
| TRIM75P | RING |
| TRIM77 | RING |
| TRIM8 | RING |
| TRIM9 | RING |
| TRIML1 | RING |
| TRIML2 | SPRY |
| TRIP12 | HECTc |
| TTC3 | RING |
| UBE3A | HECTc |
| (E3A) | |
| UBE3B | HECTc |
| UBE3C | HECTc |
| UBE3D | HECT_2 |
| UBE4A | RING |
| UBE4B | RING |
| UBOX5 | RING |
| UBR1 | UBR |
| UBR2 | UBR |
| UBR3 | UBR |
| UBR4 | UBR |
| UBR5 | HECTc |
| UBR7 | UBR |
| UHRF1 | RING |
| UHRF2 | RING |
| UNK | RING |
| UNKL | RING |
| VPS11 | RING |
| VPS18 | RING |
| VPS41 | RING |
| VPS8 | RING |
| WDR59 | RING |
| WDSUB1 | RING |
| WWP1 | HECTc |
| WWP2 | HECTc |
| XIAP | RING |
| ZBTB12 | RING |
| ZFP91 | zf-C2H2 |
| ZFPL1 | RING |
| ZNF280A | HECT |
| ZNF341 | RING |
| ZNF511 | RING |
| ZNF521 | RING |
| ZNF598 | RING |
| ZNF645 | RING |
| ZNRF1 | RING |
| ZNRF2 | RING |
| ZNRF3 | RING |
| ZNRF4 | RING |
| LNXp80 | RING |
| SNEV | UBOX |
| PIAS3 | |
| ACT1 | UBOX |

TABLE 1B
E3 ligase complex accessory proteins

| | | | | | |
|---|---|---|---|---|---|
| ABTB1 | DPF1 | FBXO8 | KCNS3 | KLHL4 | SPSB4 |
| ABTB2 | ECT2L | FBXO9 | KCNV1 | KLHL40 | TCEB3 |
| ANKFY1 | ENC1 | FBXW11 | KCTD1 | KLHL41 | TNFAIP1 |
| ARMC5 | FBXL12 | FBXW2 | KCTD10 | KLHL42 | TNFAIP3 |
| ASB10 | FBXL13 | FBXW4 | KCTD12 | KLHL5 | TULP4 |
| ASB11 | FBXL14 | FBXW5 | KCTD13 | KLHL6 | VHL |
| ASB13 | FBXL15 | FBXW7 | KCTD14 | KLHL7 | WHSC1 |
| ASB14 | FBXL16 | FBXW8 | KCTD15 | KLHL8 | WSB1 |
| ASB15 | FBXL18 | GAN | KCTD16 | KLHL9 | WSB2 |
| ASB16 | FBXL2 | GMCL1 | KCTD17 | LGALS3BP | ZBTB1 |
| ASB17 | FBXL20 | GTF2H2 | KCTD2 | LOC123103 | ZBTB10 |
| ASB18 | FBXL3 | GZF1 | KCTD20 | LRRC29 | ZBTB11 |
| ASB2 | FBXL4 | HIC1 | KCTD21 | LZTR1 | ZBTB14 |
| ASB3 | FBXL5 | HIC2 | KCTD3 | MGC23270 | ZBTB16 |
| ASB4 | FBXL6 | IBTK | KCTD4 | MYNN | ZBTB17 |
| ASB5 | FBXL7 | IPP | KCTD5 | NACC1 | ZBTB18 |
| ASB6 | FBXL8 | IVNS1ABP | KCTD6 | NACC2 | ZBTB2 |
| ASB7 | FBXO10 | KBTBD11 | KCTD7 | NEURL2 | ZBTB20 |
| ASB8 | FBXO11 | KBTBD12 | KCTD7 | OSTM1 | ZBTB21 |
| ASB9 | FBXO15 | KBTBD2 | KCTD8 | OTUD7A | ZBTB22 |
| ATRX | FBXO16 | KBTBD3 | KCTD9 | OTUD7B | ZBTB24 |
| BACH1 | FBXO17 | KBTBD4 | KDM2A | PATZ1 | ZBTB25 |
| BACH2 | FBXO18 | KBTBD6 | KDM2B | RAB40A | ZBTB26 |
| BCL6 | FBXO2 | KBTBD7 | KEAP1 | RAB40AL | ZBTB3 |
| BCL6B | FBXO21 | KBTBD8 | KLHL1 | RAB40B | ZBTB32 |
| BTBD1 | FBXO22 | KBTBD9 | KLHL10 | RAB40C | ZBTB33 |
| BTBD10 | FBXO24 | KCNA1 | KLHL11 | RABGEF1 | ZBTB37 |
| BTBD11 | FBXO25 | KCNA10 | KLHL12 | RCBTB1 | ZBTB38 |
| BTBD17 | FBXO27 | KCNA2 | KLHL13 | RCBTB2 | ZBTB39 |
| BTBD2 | FBXO28 | KCNA3 | KLHL14 | RHOBTB1 | ZBTB4 |
| BTBD3 | FBXO3 | KCNA4 | KLHL15 | RHOBTB2 | ZBTB40 |
| BTBD6 | FBXO30 | KCNA5 | KLHL17 | RHOBTB3 | ZBTB41 |
| BTBD7 | FBXO31 | KCNA6 | KLHL18 | SF3B3 | ZBTB43 |
| BTBD8 | FBXO32 | KCNA7 | KLHL20 | SHKBP1 | ZBTB44 |
| BTBD9 | FBXO33 | KCNB1 | KLHL21 | SKP2 | ZBTB45 |
| BTRC | FBXO34 | KCNB2 | KLHL22 | SLX4 | ZBTB46 |
| CCIN | FBXO36 | KCNC1 | KLHL23 | SOCS1 | ZBTB48 |
| CCNF | FBXO38 | KCNC2 | KLHL24 | SOCS2 | ZBTB49 |
| CISH | FBXO4 | KCNC3 | KLHL25 | SOCS3 | ZBTB5 |
| CPSF1 | FBXO40 | KCNC4 | KLHL26 | SOCS4 | ZBTB6 |
| CUL1 | FBXO41 | KCND1 | KLHL28 | SOCS5 | ZBTB7A |
| CUL2 | FBXO42 | KCND2 | KLHL3 | SOCS6 | ZBTB7B |
| CUL3 | FBXO44 | KCND3 | KLHL30 | SOCS7 | ZBTB7C |
| CUL4A | FBXO45 | KCNG1 | KLHL31 | SPOP | ZBTB8A |
| CUL4B | FBXO46 | KCNG3 | KLHL32 | SPOPL | ZBTB9 |
| CUL5 | FBXO5 | KCNRG | KLHL34 | SPSB1 | ZFAND3 |
| CUL7 | FBXO6 | KCNS1 | KLHL36 | SPSB2 | ZFAND4 |
| DDB1 | FBXO7 | KCNS2 | KLHL38 | SPSB3 | ZFAND5 |
| | | | | ZNF131 | ZFAND6 |

In embodiments, the degradation construct includes a targeting moiety and a degradation moiety. In embodiments, the degradation moiety includes an E3 ligase or a functional fragment thereof, or an E3 ligase recruiting moiety. As used herein an “E3 ligase or an active fragment thereof” includes E3 ligases and E3 ligase accessory proteins or active fragments thereof. An E3 ligase recruiting moiety includes a domain that binds to an E3 ligase, an E3 ligase complex, or an accessory E3 ligase protein.

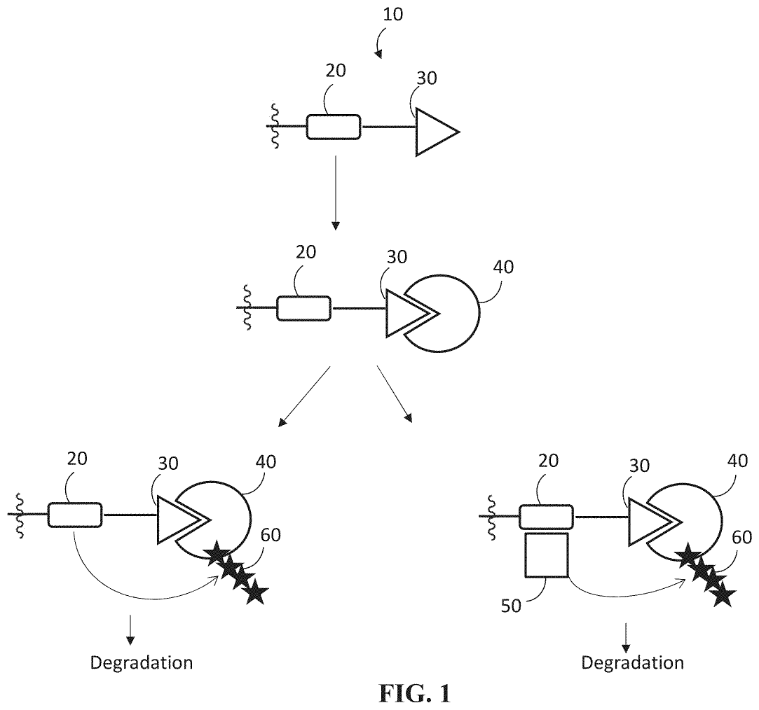

FIG. 1 schematically shows an example of how degradation compounds of the present disclosure may mediate degradation of a target protein through a ubiquitination-mediated degradation process. A degradation compound of the present disclosure is conjugated to a degradation construct 10. The degradation construct includes a targeting moiety 30 and a degradation moiety 20. The targeting moiety 30 is designed to bind to a target protein 40. The degradation moiety 20 is configured to facilitate polyubiquitylation of the target protein which leads to proteasomal degradation of the target protein. In embodiments, where the degradation moiety 20 includes an active E3 ligase or an active fragment of an active E3 ligase, the degradation moiety is directly involved in catalyzing the polyubiquitylation of the target protein. In embodiments, where the degradation moiety 20 includes an E3 ligase recruiting domain, the E3 ligase recruiting domain interacts with an endogenous E3 ligase or an endogenous E3 ligase complex 50 to catalyze the polyubiquitylation 60 of the target protein 40.

### Degradation Construct

The degradation compounds include a degradation construct The degradation construct includes a targeting moiety and a degradation moiety. The targeting moiety binds to the target protein. In embodiments, the targeting moiety includes an antibody, an antigen binding fragment, and/or an antibody mimetic.

Target Protein

The targeting moiety is capable of binding a target protein. A target protein is a protein that is being targeted for degradation by the degradation compound. In embodiments, the target protein may be described as an antigen. In embodiments, the targeting moiety is an intracellular targeting moiety, that is, the target protein is an intracellular protein.

The targeting moiety may be designed to bind any target protein of interest e.g., by appropriately screening sequences that bind to an epitope of an antigen (e.g., target protein). To screen for sequences that bind to an epitope, a cross-blocking assay can be performed such as that described in Antibodies, A Laboratory Manual, Cold Spring Harbor Laboratory, Ed Harlow and David Lane (1988), which is hereby incorporated by reference in its entirety. This assay can be used to determine if, e.g., a CDR for an antibody, or antigen binding fragment thereof binds to the same epitope of an antigen as a different antibody, or antigen binding fragment thereof. Alternatively, or additionally, epitope mapping can be performed. For example, the antibody sequence can be mutagenized, such as by alanine scanning, to identify contact residues. In a different method, peptides corresponding to different regions of the target antigen can be used in competition assays with a test antibody.

The target protein may be intracellular of extracellular protein of interests. In embodiments, the target protein is upregulated, misfolded, includes one or more mutations, has increased activity, has decreased activity, and/or has aberrant activity associated with a disease.

In embodiments, the target protein is apoptosis-associated speck-like protein (ACS) containing a C-terminal caspase recruitment domain (CARD); β-catenin; epidermal growth factor receptor (EGFR); Kristen rat sarcoma virus (KRAS; GTPase Kras); Harvery rat sarcoma virus (HRAS; GPTase HRas); NRL family pyrin domain containing 3 (NALP3 and NLRP3); In embodiments, the target protein is mouse double minute 2 homolog (MDM2); MDM4; frataxin (FXN); RAF1; and combinations thereof. Examples of some target proteins and their associated UniProt Reference numbers are provided below in Table 2. Table 3 lists some examples sequences of target proteins.

TABLE 2
Examples of Some Target Proteins

| Antigen Name | Gene ID | UniProt Ref No: |
|---|---|---|
| β-catenin | CTNNB1, CTNNB, OK/ | P35222 |
| | SW-cl35, PRO2286 | |
| Epidermal growth factor | EGFR, ERBB, ERBB1, | P00533 |
| receptor (EGFR) | HER1 | |
| GTPase KRas (KRAS) | KRAS, KRAS2, RASK2 | P01116 |
| GTPase HRas (HRAS) | HRAS, HRAS1 | P01112 |
| E3 ubiquitin-protein ligase | MDM2 | Q00987 |
| Mdm2 (MDM2) | | |
| Protein Mdm4 | MDM4 | O15151 |
| Apoptosis associated speck- | PYCARD, ASC, CARD5, | Q9ULZ3 |
| like protein containing a | TMS1 | |
| CARD (ASC) | | |
| NACHT, LRR and PYD | NLRP3, C1orf7, CIAS1, | Q96P20 |
| domains-containing protein | NALP3, PYPAF1 | |
| 3 (NALP3) | | |
| Frataxin (FXN) | FXN, FRDA, X25 | 16595 |
| RAF1 | RAF1 | P04049 |

TABLE 3
Examples of Some Target Protein Sequences

| SEQ | Target | Sequence |
|---|---|---|
| ID NO: | Protein | |
| 49 | KRAS; | MTEYKLVVVGAGGVGKSALTIQLIQNHFVDEYDPTIEDSYR |
| | aa 1-188 | KQVVIDGETCLLDILDTAGQEEYSAMRDQYMRTGEGFLCVF |
| | | AINNTKSFEDIHHYREQIKRVKDSEDVPMVLVGNKCDLPSRT |
| | | VDTKQAQDLARSYGIPFIETSAKTRQRVEDAFYTLVREIRQY |
| | | RLKKISKEEKTPGCVKIKKCII |
| 50 | KRAS | MTEYKLVVVGAVGVGKSALTIQLIQNHFVDEYDPTIEDSYR |
| | (G12V); | KQVVIDGETCLLDILDTAGQEEYSAMRDQYMRTGEGFLCVF |
| | aa 1-188 | AINNTKSFEDIHHYREQIKRVKDSEDVPMVLVGNKCDLPSRT |
| | | VDTKQAQDLARSYGIPFIETSAKTRQRVEDAFYTLVREIRQY |
| | | RLKKISKEEKTPGCVKIKKCII |
| 51 | KRAS | MTEYKLVVVGADGVGKSALTIQLIQNHFVDEYDPTIEDSYR |
| | (G12D); | KQVVIDGETCLLDILDTAGQEEYSAMRDQYMRTGEGFLCVF |
| | aa 1-188 | AINNTKSFEDIHHYREQIKRVKDSEDVPMVLVGNKCDLPSRT |
| | | VDTKQAQDLARSYGIPFIETSAKTRQRVEDAFYTLVREIRQY |
| | | RLKKISKEEKTPGCVKIKKCII |
| 52 | KRAS | MTEYKLVVVGAGDVGKSALTIQLIQNHFVDEYDPTIEDSYR |
| | (G13D); aa | KQVVIDGETCLLDILDTAGQEEYSAMRDQYMRTGEGFLCVF |
| | 1-185 | AINNTKSFEDIHHYREQIKRVKDSEDVPMVLVGNKCDLPSRT |
| | | VDTKQAQDLARSYGIPFIETSAKTRQRVEDAFYTLVREIRQY |
| | | RLKKISKEEKTPGCVKIKK |
| 53 | HRAS(HRA | MTEYKLVVVGAGGVGKSALTIQLIQNHFVDEYDPTIEDSYR |
| | S(WT)) | KQVVIDGETCLLDILDTAGQEEYSAMRDQYMRTGEGFLCVF |
| | | AINNTKSFEDIHQYREQIKRVKDSDDVPMVLVGNKCDLAAR |
| | | TVESRQAQDLARSYGIPYIETSAKTRQGVEDAFYTLVREIRQ |
| | | HKLRKLNPPDESGPGCMSCKCVLS |
| 54 | HRAS | MTEYKLVVVGAVGVGKSALTIQLIQNHFVDEYDPTIEDSYR |
| | (G12V) | KQVVIDGETCLLDILDTAGQEEYSAMRDQYMRTGEGFLCVF |
| | | AINNTKSFEDIHQYREQIKRVKDSDDVPMVLVGNKCDLAAR |
| | | TVESRQAQDLARSYGIPYIETSAKTRQGVEDAFYTLVREIRQ |
| | | HKLRKLNPPDESGPGCMSCKCVLS |
| 55 | HRAS | MTEYKLVVVGADGVGKSALTIQLIQNHFVDEYDPTIEDSYR |
| | (G12D) | KQVVIDGETCLLDILDTAGQEEYSAMRDQYMRTGEGFLCVF |
| | | AINNTKSFEDIHQYREQIKRVKDSDDVPMVLVGNKCDLAAR |
| | | TVESRQAQDLARSYGIPYIETSAKTRQGVEDAFYTLVREIRQ |
| | | HKLRKLNPPDESGPGCMSCKCVLS |
| 56 | MDM2; | MCNTNMSVPTDGAVTTSQIPASEQETLVRPKPLLLKLLKSVG |
| | aa 1-110 | AQKDTYTMKEVLFYLGQYIMTKRLYDEKQQHIVYCSNDLL |
| | | GDLFGVPSFSVKEHRKIYTMIYRNLVVV |
| 57 | MDM4; | MTSFSTSAQCSTSDSACRISPGQINQVRPKLPLLKILHAAGAQ |
| | aa 1-178 | GEMFTVKEVMHYLGQYIMVKQLYDQQEQHMVYCGGDLLG |
| | | ELLGRQSFSVKDPSPLYDMLRKNLVTLATATTDAAQTLALA |
| | | QDHSMDIPSQDQLKQSAEESSTSRKRTTEDDIPTLPTSEHKCI |
| | | HSREDEDLIENL |
| 58 | RAF1 | MEHIQGAWKTISNGFGFKDAVFDGSSCISPTIVQQFGYQRRA |
| | | SDDGKLTDPSKTSNTIRVFLPNKQRTVVNVRNGMSLHDCLM |
| | | KALKVRGLQPECCAVFRLLHEHKGKKARLDWNTDAASLIGE |
| | | ELQVDFLDHVPLTTHNFARKTFLKL |
| 59 | β-catenin | MATQADLMELDMAMEPDRKAAVSHWQQQSYLDSGIHSGA |
| | | TTTAPSLSGKGNPEEEDVDTSQVLYEWEQGFSQSFTQEQVA |
| | | DIDGQYAMTRAQRVRAAMFPETLDEGMQIPSTQFDAAHPTN |
| | | VQRLAEPSQMLKHAVVNLINYQDDAELATRAIPELTKLLND |
| | | EDQVVVNKAAVMVHQLSKKEASRHAIMRSPQMVSAIVRTM |
| | | QNTNDVETARCTAGTLHNLSHHREGLLAIFKSGGIPALVKML |
| | | GSPVDSVLFYAITTLHNLLLHQEGAKMAVRLAGGLQKMVA |
| | | LLNKTNVKFLAITTDCLQILAYGNQESKLIILASGGPQALVNI |
| | | MRTYTYEKLLWTTSRVLKVLSVCSSNKPAIVEAGGMQALGL |
| | | HLTDPSQRLVQNCLWTLRNLSDAATKQEGMEGLLGTLVQLL |
| | | GSDDINVVTCAAGILSNLTCNNYKNKMMVCQVGGIEALVRT |
| | | VLRAGDREDITEPAICALRHLTSRHQEAEMAQNAVRLHYGL |
| | | PVVVKLLHPPSHWPLIKATVGLIRNLALCPANHAPLREQGAI |
| | | PRLVQLLVRAHQDTQRRTSMGGTQQQFVEGVRMEEIVEGCT |
| | | GALHILARDVHNRIVIRGLNTIPLFVQLLYSPIENIQRVAAGVL |
| | | CELAQDKEAAEAIEAEGATAPLTELLHSRNEGVATYAAAVL |
| | | FRMSEDKPQDYKKRLSVELTSSLFRTEPMAWNETADLGLDI |
| | | GAQGEPLGYRQDDPSYRSFHSGGYGQDALGMDPMMEHEM |
| | | GGHHPGADYPVDGLPDLGHAQDLMDGLPPGDSNQLAWFDT |
| | | DL |
| 60 | Human | MKMASTRCKLARYLEDLEDVDLKKFKMHLEDYPPQKGCIPL |
| | NLRP3 | PRGQTEKADHVDLATLMIDFNGEEKAWAMAVWIFAAINRR |
| | | DLYEKAKRDEPKWGSDNARVSNPTVICQEDSIEEEWMGLLE |
| | | YLSRISICKMKKDYRKKYRKYVRSRFQCIEDRNARLGESVSL |
| | | NKRYTRLRLIKEHRSQQEREQELLAIGKTKTCESPVSPIKMEL |
| | | LFDPDDEHSEPVHTVVFQGAAGIGKTILARKMMLDWASGTLY |
| | | QDRFDYLFYIHCREVSLVTQRSLGDLIMSCCPDPNPPIHKIV |
| | | RKPSRILFLMDGFDELQGAFDEHIGPLCTDWQKAERGDILLS |
| | | SLIRKKLLPEASLLITTRPVALEKLQHLLDHPRHVEILGFSE |
| | | AKRKEYFFKYFSDEAQARAAFSLIQENEVLFTMCFIPLVCWI |
| | | VCTGLKQQMESGKSLAQTSKTTTAVYVFFLSSLLQPRGGSQE |
| | | HGLCAHLWGLCSLAADGIWNQKILFEESDLRNHGLQKADVSAF |
| | | LRMNLFQKEVDCEKFYSFIHMTFQEFFAAMYYLLEEEKEGR |
| | | TNVPGSRLKLPSRDVTVLLENYGKFEKGYLIFVVRFLFGLVN |
| | | QERTSYLEKKLSCKISQQIRLELLKWIEVKAKAKKLQIQPSQL |
| | | ELFYCLYEMQEEDFVQRAMDYFPKIEINLSTRMDHMVSSFCI |
| | | ENCHRVESLSLGFLHNMPKEEEEEEKEGRHLDMVQCVLPSSS |
| | | HAACSHGLVNSHLTSSFCRGLFSVLSTSQSLTELDLSDNSLGD |
| | | PGMRVLCETLQHPGCNIRRLWLGRCGLSHECCFDISLVLSSN |
| | | QKLVELDLSDNALGDFGIRLLCVGLKHLLCNLKKLWLVSCC |
| | | LTSACCQDLASVLSTSHSLTRLYVGENALGDSGVAILCEKAK |
| | | NPQCNLQKLGLVNSGLTSVCCSALSSVLSTNQNLTHLYLRG |
| | | NTLGDKGIKLLCEGLLHPDCKLQVLELDNCNLTSHCCWDLS |
| | | TLLTSSQSLRKLSLGNNDLGDLGVMMFCEVLKQQSCLLQNL |
| | | GLSEMYFNYETKSALETLQEEKPELTVVFEPSW |
| 61 | Mouse | MTSVRCKLAQYLEDLEDVDLKKFKMHLEDYPPEKGCIPVPR |
| | NLRP3 | GQMEKADHLDLATLMIDFNGEEKAWAMAVWIFAAINRRDL |
| | | WEKAKKDQPEWNDTCTSHSSMVCQEDSLEEEWMGLLGYLS |
| | | RISICKKKKDYCKMYRRHVRSRFYSIKDRNARLGESVDLNSR |
| | | YTQLQLVKEHPSKQEREHELLTIGRTKMRDSPMSSLKLELLF |
| | | EPEDGHSEPVHTVVFQGAAGIGKTILARKIMLDWALGKLFK |
| | | DKFDYLFFIHCREVSLRTPRSLADLIVSCWPDPNPPVCKILRK |
| | | PSRILFLMDGFDELQGAFDEHIGEVCTDWQKAVRGDILLSSLI |
| | | RKKLLPKASLLITTRPVALEKLQHLLDHPRHVEILGFSEAKRK |
| | | EYFFKYFSNELQAREAFRLIQENEVLFTMCFIPLVCWIVCTGL |
| | | KQQMETGKSLAQTSKTTTAVYVFFLSSLLQSRGGIEEHLFSD |
| | | YLQGLCSLAADGIWNQKILFEECDLRKHGLQKTDVSAFLRM |
| | | NVFQKEVDCERFYSFSHMTFQEFFAAMYYLLEEEAEGETVR |
| | | KGPGGCSDLLNRDVKVLLENYGKFEKGYLIFVVRFLFGLVN |
| | | QERTSYLEKKLSCKISQQVRLELLKWIEVKAKAKKLQWQPS |
| | | QLELFYCLYEMQEEDFVQSAMDHFPKIEINLSTRMDHVVSSF |
| | | CIKNCHRVKTLSLGFFHNSPKEEEEERRGGRPLDQVQCVFPD |
| | | THVACSSRLVNCCLTSSFCRGLFSSLSTNRSLTELDLSDNTLG |
| | | DPGMRVLCEALQHPGCNIQRLWLGRCGLSHQCCFDISSVLSS |
| | | SQKLVELDLSDNALGDFGIRLLCVGLKHLLCNLQKLWLVSC |
| | | CLTSACCQDLALVLSSNHSLTRLYIGENALGDSGVQVLCEK |
| | | MKDPQCNLQKLGLVNSGLTSICCSALTSVLKTNQNFTHLYLR |
| | | SNALGDTGLRLLCEGLLHPDCKLQMLELDNCSLTSHSCWNL |
| | | STILTHNHSLRKLNLGNNDLGDLCVVTLCEVLKQQGCLLQSL |
| | | QLGEMYLNRETKRALEALQEEKPELTIVFEISW |
| 62 | Human | MKMASTRCKLARYLEDLEDVDLKKFKMHLEDYPPQKGCIPL |
| | NLRP3; | PRGQTEKADHVDLATLMIDFNGEEKAWAMAVWIFAAINRR |
| | PYD domain | DLYEKAKRDEPK |
| 63 | Mouse | MTSVRCKLAQYLEDLEDVDLKKFKMHLEDYPPEKGCIPVPR |
| | NLRP3; | GQMEKADHLDLATLMIDFNGEEKAWAMAVWIFAAINRRDL |
| | PYD domain | WEKAKKDQPE |
| 64 | Human ASC | MGRARDAILDALENLTAEELKKFKLKLLSVPLREGYGRIPRG |
| | | ALLSMDALDLTDKLVSFYLETYGAELTANVLRDMGLQEMA |
| | | GQLQAATHQGSGAAPAGIQAPPQSAAKPGLHFIDQHRAALIA |
| | | RVTNVEWLLDALYGKVLTDEQYQAVRAEPTNPSKMRKLFS |
| | | FTPAWNWTCKDLLLQALRESQSYLVEDLERS |
| 65 | Mouse ASC | MGRARDAILDALENLSGDELKKFKMKLLTVQLREGYGRIPR |
| | | GALLQMDAIDLTDKLVSYYLESYGLELTMTVLRDMGLQELA |
| | | EQLQTTKEESGAVAAAASVPAQSTARTGHFVDQHRQALIAR |
| | | VTEVDGVLDALHGSVLTEGQYQAVRAETTSQDKMRKLFSF |
| | | VPSWNLTCKDSLLQALKEIHPYLVMDLEQS |
| 66 | Human | MRPSGTAGAALLALLAALCPASRALEEKKVCQGTSNKLTQL |
| | EGFR | GTFEDHFLSLQRMFNNCEVVLGNLEITYVQRNYDLSFLKTIQ |
| | | EVAGYVLIALNTVERIPLENLQIIRGNMYYENSYALAVLSNY |
| | | DANKTGLKELPMRNLQEILHGAVRFSNNPALCNVESIQWRDI |
| | | VSSDFLSNMSMDFQNHLGSCQKCDPSCPNGSCWGAGEENC |
| | | QKLTKIICAQQCSGRCRGKSPSDCCHNQCAAGCTGPRESDCL |
| | | VCRKFRDEATCKDTCPPLMLYNPTTYQMDVNPEGKYSFGAT |
| | | CVKKCPRNYVVTDHGSCVRACGADSYEMEEDGVRKCKKCEGP |
| | | CRKVCNGIGIGEFKDSLSINATNIKHFKNCTSISGDLHILPV |
| | | AFRGDSFTHTPPLDPQELDILKTVKEITGFLLIQAWPENRTDL |
| | | HAFENLEIIRGRTKQHGQFSLAVVSLNITSLGLRSLKEISDGD |
| | | VIISGNKNLCYANTINWKKLFGTSGQKTKIISNRGENSCKATG |
| | | QVCHALCSPEGCWGPEPRDCVSCRNVSRGRECVDKCNLLEG |
| | | EPREFVENSECIQCHPECLPQAMNITCTGRGPDNCIQCAHYID |
| | | GPHCVKTCPAGVMGENNTLVWKYADAGHVCHLCHPNCTY |
| | | GCTGPGLEGCPTNGPKIPSIATGMVGALLLLLVVALGIGLFM |
| | | RRRHIVRKRTLRRLLQERELVEPLTPSGEAPNQALLRILKETE |
| | | FKKIKVLGSGAFGTVYKGLWIPEGEKVKIPVAIKELREATSPK |
| | | ANKEILDEAYVMASVDNPHVCRLLGICLTSTVQLITQLMPFG |
| | | CLLDYVREHKDNIGSQYLLNWCVQIAKGMNYLEDRRLVHR |
| | | DLAARNVLVKTPQHVKITDFGLAKLLGAEEKEYHAEGGKVPI |
| | | KWMALESILHRIYTHQSDVWSYGVTVWELMTFGSKPYDGIPA |
| | | SEISSILEKGERLPQPPICTIDVYMIMVKCWMIDADSRPKFR |
| | | ELIIEFSKMARDPQRYLVIQGDERMHLPSPTDSNFYRALMDE |
| | | EDMDDVVDADEYLIPQQGFFSSPSTSRTPLLSSLSATSNNSTV |
| | | ACIDRNGLQSCPIKEDSFLQRYSSDPTGALTEDSIDDTFLPVP |
| | | EYINQSVPKRPAGSVQNPVYHNQPLNPAPSRDPHYQDPHSTA |
| | | VGNPEYLNTVQPTCVNSTFDSPAHWAQKGSHQISLDNPDYQ |
| | | QDFFPKEAKPNGIFKGSTAENAEYLRVAPQSSEFIGA |

In some embodiments, the target protein is interferon regulatory factor 5 (IRF-5). TRF-5 is a member of the TRF family of transcription factors and is involved in innate and adaptive immunity, macrophage polarization, cell growth regulation and differentiation, antiviral defense, the production of proinflammatory cytokines, and apoptosis.

IRF-5 exists in multiple isoforms that are generated by three alternative non-coding 5′ exons and at least nine alternatively spliced mRNAs. The sequences for the TRF-5 isoforms are publicly available, for example, through the online UniProt database. The isoforms show cell-type specific expression, subcellular localization and function. Some isoforms are associated with risk of autoimmune disease. For example, Isoform 2 is linked to overexpression of IRF-5 and susceptibility to autoimmune disease such as systemic lupus erythematosus.

The gene encoding IRF-5 includes 9 exons (exon 1, exon 2, exon 3, exon 4, exon 5, exon 6, exon 7, exon 8, and exon 9. Exon 1 is in the 5′-untranslated region (5′-UTR) and has three variants, exon 1A, exon 1B, exon 1C, and exon 1D. The predominant isoform includes Exon 1A. Exon 1B is associated with IRF-5 hyperactivation and disease progression. Single-nucleotide polymorphisms (SNP) (e.g., rs2004640) that introduce a donor splice site can lead to increased expression of Exon 1B transcripts and reduced expression of Exon 1C-derived transcripts. Other SNPs (e.g., rs2280714) are also associated with elevated IRF-5 expression (Kozyrev et al., Arthritis and Rheumatology. (2007), 56(4):1234-1241).

In embodiments, the degradation compound mediates degradation of one or more isoform of IRF-5. In embodiments, the degradation compound mediates degradation of IRF-5 having an amino acid sequence of IRF-5 Isoform 1. In embodiments, the degradation compound mediates degradation of IRF-5 having an amino acid sequence of IRF-5 Isoform 2. In embodiments, the degradation compound mediates degradation of IRF-5 having an amino acid sequence of IRF-5 Isoform 3. In embodiments, the degradation compound mediates degradation of IRF-5 having an amino acid sequence of IRF-5 Isoform 4. In embodiments, the degradation compound mediates degradation of IRF-5 having an amino acid sequence of IRF-5 Isoform 5. In embodiments, the degradation compound mediates degradation of IRF-5 having an amino acid sequence of IRF-5 Isoform 6. In embodiments, the degradation compound mediates degradation of IRF-5 having an amino acid that differs by one or more amino acids of IRF-5 Isoform 1, IRF-5 Isoform 2, IRF-5 Isoform 3, IRF-5 Isoform 4, IRF-5 Isoform 5, or IRF-5 Isoform 6. In embodiments, the degradation compound mediates degradation of IRF-5 having an amino acid sequence that shares 100% identity to an amino acid sequence of IRF-5 Isoform 1, IRF-5 Isoform 2, IRF-5 Isoform 3, IRF-5 Isoform 4, IRF-5 Isoform 5, or IRF-5 Isoform 6. In embodiments, the degradation compound mediates degradation of IRF-5 having an amino acid sequence that shares less than 100% identity to an amino acid sequence of IRF-5 Isoform 1, IRF-5 Isoform 2, IRF-5 Isoform 3, IRF-5 Isoform 4, IRF-5 Isoform 5, or IRF-5 Isoform 6. In embodiments, the degradation compound mediates degradation of IRF-5 having an amino acid sequence that is at least about 80%, about 85%, about 90%, about 91%, about 92%, about 93%, about 94%, about 95%, about 96%, about 97%, about 98%, or about 99% identical to an amino acid sequence of IRF-5 Isoform 1, IRF-5 Isoform 2, IRF-5 Isoform 3, IRF-5 Isoform 4, IRF-5 Isoform 5, or IRF-5 Isoform 6. The sequences of TRF-5 Isoform 1, IRF-5 Isoform 2, TRF-5 Isoform 3, IRF-5 Isoform 4, TRF-5 Isoform 5, or TRF-5 Isoform 6 are provided below:

HUMAN Interferon regulatory factor-5
(IRF-5) (Isoform 1)
(SEQ ID NO: 67)
MNQSIPVAPTPPRRVRLKPWLVAQVNSCQYPGLQW
VNGEKKLFCIPWRHATRHGPSQDGDNTIFKAWAKE
TGKYTEGVDEADPAKWKANLRCALNKSRDFRLIYD
GPRDMPPQPYKIYEVCSNGPAPTDSQPPEDYSFGA
GEEEEEEEELQRMLPSLSLTEDVKWPPTLQPPTLR
PPTLQPPTLQPPVVLGPPAPDPSPLAPPPGNPAGF
RELLSEVLEPGPLPASLPPAGEQLLPDLLISPHML
PLTDLEIKFQYRGRPPRALTISNPHGCRLFYSQLE
ATQEQVELFGPISLEQVRFPSPEDIPSDKQRFYTN
QLLDVLDRGLILQLQGQDLYAIRLCQCKVFWSGPC
ASAHDSCPNPIQREVKTKLFSLEHFLNELILFQKG
QTNTPPPFEIFFCFGEEWPDRKPREKKLITVQVVP
VAARLLLEMFSGELSWSADSIRLQISNPDLKDRMV
EQFKELHHIWQSQQRLQPVAQAPPGAGLGVGQGPW
PMHPAGM,
HUMAN Interferon regulatory factor-5
(IRF-5) (Isoform 2)
(SEQ ID NO: 68)
MNQSIPVAPTPPRRVRLKPWLVAQVNSCQYPGLQW
VNGEKKLFCIPWRHATRHGPSQDGDNTIFKAWAKE
TGKYTEGVDEADPAKWKANLRCALNKSRDFRLIYD
GPRDMPPQPYKIYEVCSNGPAPTDSQPPEDYSFGA
GEEEEEEEELQRMLPSLSLTDAVQSGPHMTPYSLL
KEDVKWPPTLQPPTLRPPTLQPPTLQPPVVLGPPA
PDPSPLAPPPGNPAGFRELLSEVLEPGPLPASLPP
AGEQLLPDLLISPHMLPLTDLEIKFQYRGRPPRAL
TISNPHGCRLFYSQLEATQEQVELFGPISLEQVRF
PSPEDIPSDKQRFYTNQLLDVLDRGLILQLQGQDL
YAIRLCQCKVFWSGPCASAHDSCPNPIQREVKTKL
FSLEHFLNELILFQKGQTNTPPPFEIFFCFGEEWP
DRKPREKKLITVQWPVAARLLLEMFSGELSWSADS
IRLQISNPDLKDRMVEQFKELHHIWQSQQRLQPVA
QAPPGAGLGVGQGPWPMHPAGMQ,
HUMAN Interferon regulatory factor-5
(IRF-5) (Isoform 3)
(SEQ ID NO: 69)
MNQSIPVAPTPPRRVRLKPWLVAQVNSCQYPGLQW
VNGEKKLFCIPWRHATRHGPSQDGDNTIFKAWAKE
TGKYTEGVDEADPAKWKANLRCALNKSRDFRLIYD
GPRDMPPQPYKIYEVCSNGPAPTDSQPPEDYSFGA
GEEEEEEEELQRMLPSLSLTDAVQSGPHMTPYSLL
KEDVKWPPTLQPPTLQPPVVLGPPAPDPSPLAPPP
GNPAGFRELLSEVLEPGPLPASLPPAGEQLLPDLL
ISPHMLPLTDLEIKFQYRGRPPRALTISNPHGCRL
FYSQLEATQEQVELFGPISLEQVRFPSPEDIPSDK
QRFYTNQLLDVLDRGLILQLQGQDLYAIRLCQCKV
FWSGPCASAHDSCPNPIQREVKTKLFSLEHFLNEL
ILFOKGOTNTPPPFEIFFCFGEEWPDRKPREKKLI
TVQVVPVAARLLLEMFSGELSWSADSIRLQISNPD
LKDRMVEQFKELHHIWQSQQRLQPVAQAPPGAGLG
VGQGPWPMHPAGMQ,
HUMAN Interferon regulatory factor-5
(IRF-5) (Isoform 4)
(SEQ ID NO: 70)
MNQSIPVAPTPPRRVRLKPWLVAQVNSCQYPGLQW
VNGEKKLFCIPWRHATRHGPSQDGDNTIFKAWAKE
TGKYTEGVDEADPAKWKANLRCALNKSRDFRLIYD
GPRDMPPQPYKIYEVCSNGPAPTDSQPPEDYSFGA
GEEEEEEEELQRMLPSLSLTEDVKWPPTLQPPTLQ
PPVVLGPPAPDPSPLAPPPGNPAGFRELLSEVLEP
GPLPASLPPAGEQLLPDLLISPHMLPLTDLEIKFQ
YRGRPPRALTISNPHGCRLFYSQLEATQEQVELFG
PISLEQVRFPSPEDIPSDKQRFYTNQLLDVLDRGL
ILQLQGQDLYAIRLCQCKVFWSGPCASAHDSCPNP
IQREVKTKLFSLEHFLNELILFQKGQTNTPPPFEI
FFCFGEEWPDRKPREKKLITVQVVPVAARLLLEMF
SGELSWSADSIRLQISNPDLKDRMVEQFKELHHIW
QSQQRLQPVAQAPPGAGLGVGQGPWPMHPAGMQ,
HUMAN Interferon regulatory factor-5
(IRF-5) (Isoform 5)
(SEQ ID NO: 71)
MNQSIPVAPTPPRRVRLKPWLVAQVNSCQYPGLQW
VNGEKKLFCIPWRHATRHGPSQDGDNTIFKAWAKE
TGKYTEGVDEADPAKWKANLRCALNKSRDFRLIYD
GPRDMPPQPYKIYEVCSNGPAPTDSQPPEDYSFGA
GEEEEEEEELQRMLPSLSLTVTDLEIKFQYRGRPP
RALTISNPHGCRLFYSQLEATQEQVELFGPISLEQ
VRFPSPEDIPSDKQRFYTNQLLDVLDRGLILQLQG
QDLYAIRLCQCKVFWSGPCASAHDSCPNPIQREVK
TKLFSLEHFLNELILFQKGQTNTPPPFEIFFCFGE
EWPDRKPREKKLITVQVVPVAARLLLEMFSGELSW
SADSIRLQISNPDLKDRMVEQFKELHHIWQSQORL
QPVAQAPPGAGLGVGQGPWPMHPAGMQ,
and
HUMAN Interferon regulatory factor-5
(IRF-5) (Isoform 6)
(SEQ ID NO: 72)
MNQSIPVAPTPPRRVRLKPWLVAQVNSCQYPGLQW
VNGEKKLFCIPWRHATRHGPSQDGDNTIFKAWAKE
TGKYTEGVDEADPAKWKANLRCALNKSRDFRLIYD
GPRDMPPQPYKIYETPSPLRITLLVQERRRKKRKS
CRGCCQA.

As used herein, a polypeptide is “structurally similar” to a reference polypeptide if the amino acid sequence of the polypeptide possesses a specified amount of identity compared to the reference polypeptide. Structural similarity of two polypeptides can be determined by aligning the residues of the two polypeptides (for example, a candidate polypeptide and the polypeptide of, for example, KRas^{VHH}-hFc (N297A/H433A) or Human EGFR) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate polypeptide is the polypeptide being compared to the reference polypeptide (e.g., KRas^{VHH}-hFc (N297A/H433A) or Human EGFR). A candidate polypeptide can be isolated, for example, from an animal, or can be produced using recombinant techniques, or chemically or enzymatically synthesized.

A pair-wise comparison analysis of amino acid sequences can be carried out using the BESTFIT algorithm in the GCG package (version 10.2, Madison Wis.). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide may be selected from other members of the class to which the amino acid belongs.

For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, or glutamine. The positively charged (basic) amino acids include arginine, lysine, or histidine. The negatively charged (acidic) amino acids include aspartic acid or glutamic acid. Conservative substitutions include, for example, Lys for Arg or vice versa to maintain a positive charge; Glu for Asp or vice versa to maintain a negative charge; Ser for Thr or vice versa so that a free —OH is maintained; or Gln for Asn or vice versa to maintain a free —NH₂. Likewise, biologically active analogs of a polypeptide containing deletions or additions of one or more contiguous or noncontiguous amino acids that do not eliminate a functional activity of the polypeptide are also contemplated.

In some embodiments, the target protein comprises an amino acid sequence that has at least 80%, 85%, 90%, 91%, 92%, 93%, 94%, 95%, 96%, 97%, 98%, or 99% percent similarity and/or percent identity to any of the example target proteins listed in Table 2 or Table 3 or IRF-5 isoforms 1 to 6. In embodiments, the target protein has 80% to 100%, 90% to 100%, 95% to 100%, or 99% to 100% percent similarity and/or percent identity to any of the exemplary target proteins listed in Table 2 or Table 3 or IRF-5 isoforms 1 to 6.

### Targeting Moiety

The degradation construct of the degradation compound includes a targeting moiety that binds to the target protein. In embodiments, the targeting moiety includes an antibody, and antigen binding fragment, and/or an antibody mimetic. In embodiments, the targeting moiety includes two targeting moieties that bind two different epitopes on the target protein.

Antibodies and Antigen Binding Fragments

In embodiments, the targeting moiety includes and antibody or an antigen binding fragment. The terms “antibody” and antigen binding fragment overlap to some extent. The term antibody includes any full-length antibody or fragment of an antibody capable of binding to a particular antigen including a molecule that immunospecifically binds to a particular antigen of interest. In contrast, an antigen binding fragment refers to a polypeptide fragment that includes at least one complementarity-determining region (CDR) of an antibody. For example, Fab, F(ab′)2, Fab′, Fv fragments, minibodies, single domain antibodies (sdAb), single-chain variable fragments (scFv), divalent scFv such as diabodies, multispecific antibodies formed from antibody fragments are both antibodies and antigen binding fragments. In contrast, an Fc and pFc′ are antibodies but no antigen binding fragments as they do not include a CDR.

In embodiments, the antibody is an IgG (IgG1, IgG2, IgG3, IgG4, IgA1, IgA2), IgE, IgD, IgY, or combinations thereof. In embodiments, the antibody is a full-length monoclonal antibody or polyclonal antibody. In embodiments, the antibody is a single chain antibody (scAb). In embodiments, the single chain antibody is a camelid antibody. In embodiments, the targeting moiety is a Fab, F(ab′)2, Fab′, Fv fragment, minibody, sdAb, scFv, divalent scFv, diabobody, triabody, multispecific antibody, single domain antibody (sdAb), or combinations thereof.

Known antibodies and antigen binding fragments thereof that specifically bind the target antigens described herein (e.g., ASC, β-catenin, EGFR, KRAS, HRAS, MDM2, MDM4, NALP3, and FXN) that are known in the art and can be incorporated into the constructs described herein. For example, ASC antibodies and binding fragments are described in US20190002550 and are commercially available from Santa Cruz Biotechnology (e.g., Product No. sc-514414) and Abcam (e.g., Product Nos. ab155970, ab175449, ab180799, ab151700, and ab249023). β-catenin antibodies and binding fragments are commercially available from Abcam (e.g., Product #s ab32572, ab16051, ab196204), Cell Signaling (e.g., Product Nos. 8480, 19807, 4176, 9587, 9562), and Santa Cruz Biotechnologies (e.g., Product No. sc-7963). EGFR antibodies and binding fragments are described in US20150337042, US20140286969, US20150030599, U.S. Pat. Nos. 9,789,184, 9,226,964, and 9,458,236 and are commercially available from R&D Systems (e.g., Product No. AF231), Abcam (e.g., Product Nos. ab52894, ab32077, ab32198, ab32562), and Invitrogen (e.g., Product Nos. MA5-13070, PA1-1110, and MA5-13269). KRAS antibodies and binding fragments are described in US20190284275 and are commercially available from Abcam (e.g., Product Nos. ab275876 and ab180772), Novus Biologicals (e.g., Product Nos. NBP2-45535), and ThermoFisher Scientific (e.g., Product No. CF801672). HRAS antibodies are commercially available from Abcam (e.g., Product No. ab32417), Novus Biologicals (e.g., Product No. NBP2-42864), Sigma-Aldrich (e.g., Product No. SAB4700841-100UG), and Santa Cruz Biotechnology (e.g., ProductNo. sc-35). MDM2 antibodies and binding fragments are described in U.S. Pat. No. 7,304,142B2 and WO2002004601 and are commercially available from ThermoFisher Scientific (e.g., Product Nos. MA1-113, 700555, and PA5-11353), Abcam (e.g., Product Nos. ab16895, ab259265, and ab226939), and R&D Systems (e.g., Product Nos. MAB1244 and AF1244). MDM4 antibodies and binding fragments are commercially available from Abcam (e.g., Product Nos. ab243859, ab49993, and ab16058), Proteintech (e.g., Product No. 17914-1-AP), and Invitrogen (e.g., Product Nos. MA5-26198, MA5-15432, MA5-26195, MA5-32694, and PA5-102843). NALP3 antibodies and binding fragments are commercially available from Invitrogen (e.g., Product Nos. MA5-32255, PA5-109211, PA5-79740, and MA5-23919), Abcam (e.g., Product Nos. ab263899, ab270449, and ab260017), and R&D Systems (e.g., Product Nos. AF7010 and MAB6789). FXN antibodies and binding fragments and are commercially available from Abcam (e.g., Product Nos. ab219414, ab113691, ab110329, and ab124680), Novus Biologicals (e.g., Product No. NBP2-01743), and Invitrogen (e.g., Product Nos. 45-6300, 14147-1-AP, CF504254, and TA504254).

In embodiments, the antibody or antigen binding fragment sequence includes a C-terminal and/or N-terminal cysteine to provide a site for conjugation to the EEV or an additional targeting moiety as described herein. The addition of a C-terminal and/or N-terminal cysteine may be denoted by a -cys (or -Cys) on the N-terminal and/or C-terminal end of the antibody or antigen binding fragment construct.

In some embodiments, the targeting moiety includes a single chain variable fragment (scFvs). An scFv refers to a fusion protein of the variable regions of the heavy (VH) and light chains (VL) of immunoglobulins, connected with a short linker peptide of ten to about 25 amino acids (Huston et al. Proc. Nat. Acad. Sci. USA (1988), 85(16):5879-5883). The linker can connect the N-terminus of the VH with the C-terminus of the VL, or vice versa. A number of methods have been described to discern chemical structures for converting the naturally aggregated, but chemically separated, light and heavy polypeptide chains from an antibody V region into an scFv molecule which will fold into a three-dimensional structure substantially similar to the structure of an antigen-binding site (See, e.g., U.S. Pat. Nos. 5,091,513; 5,132,405; and U.S. Pat. No. 4,946,778).

In some embodiments, the targeting moiety is a diabody. The term diabody refers to a bispecific antibody fragment in which VH and VL domains are expressed in a single polypeptide chain using a linker that is too short to allow for pairing between the two domains on the same chain, thereby forcing the domains to pair with complementary domains of another chain and creating two antigen-binding sites (see, e.g., Holliger et al., Proc. Natl. Acad. Sci. USA 90:6444-48 (1993) and Poljak et al., Structure 2:1121-23 (1994)). In embodiments, diabodies may be designed to bind to two distinct antigens and are bi-specific antigen-binding constructs.

In some embodiments, the targeting moiety includes a minibody. Minibodies (Mb) comprise a CH3 domain fused or linked to an antigen-binding fragment (e.g., a CH3 domain fused or linked to an scFv, a single domain antibody, etc.). In embodiments, the term “Mb” signifies a CH3 domain. In embodiments, a CH3 domain signifies a minibody (See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996).

In some embodiments, the targeting domain includes a “dual-variable-domain-IgG” or “DVD-IgG”. DVD-IgGs are generated from two parental monoclonal antibodies by fusing VL and VH domains of IgG with one specificity to the N-terminal of VL and VH of an IgG of different specificity, respectively, via a linker sequence.

In some embodiments, the targeting moiety includes a single domain antibody (sdAb) also sometimes called a NANOBODY. Single domain antibodies refer to a single monomeric variable antibody domain comprising one variable domain (VH) of a heavy-chain antibody such as an IgG or a scAb from a camelid. In cases where the sdAb is derived from a camelid scAb, the variable domain is the VHH of a scAb. As such, a sdAb may be referred to as simply VHH. Single domain antibodies possess several advantages over traditional monoclonal antibodies (mAbs), including smaller size (˜15 kD), stability in the reducing intracellular environment, and ease of production in bacterial systems (Schumacher et al., (2018) Angew. Chem. Int. Ed. 57, 2314; Siontorou, (2013) International Journal of Nanomedicine, 8, 4215-27). These features render sdAbs amendable to genetic and chemical modifications (Schumacher et al., (2018) Angew. Chem. Int. Ed. 57, 2314), facilitating their application as research tools and therapeutic agents (Bannas et al., (2017) Frontiers in Immunology, 8, 1603). Over the past decade, sdAbs have been used for protein immobilization (Rothbauer et al., (2008) Mol. Cell. Proteomics, 7, 282-289), imaging (Traenkle et al., (2015) Mol. Cell. Proteomics, 14, 707-723), detection of protein-protein interactions (Herce et al., (2013) Nat. Commun, 4, 2660; Massa et al., (2014) Bioconjugate Chem, 25, 979-988), and as macromolecular inhibitors (Truttmann et al., (2015) J. Biol. Chem. 290, 9087-9100).

In embodiments, the targeting moiety is a sdAb derived from a camelid scAb. In embodiments, the targeting moiety is a sdAb including the VHH derived from a camelid scAb. In embodiments, the targeting moiety is a sdAb including the three CDRs of a VHH derived from a camelid scAb.

In embodiments, the targeting moieties comprise single-domain antibodies that specifically bind to a target antigen selected from β-catenin, EGFR, KRAS, HRAS, or ASC.

In embodiments, the targeting moiety comprises a single-domain antibody that specifically binds to β-catenin. In embodiments, the single domain antibody that specifically binds to β-catenin is BC1; BC2; BC3; BC4; BC5; BC6; BC7; BC8; BC9; BC10; BC11; BC12; BC13; BC14; or combinations thereof. In embodiments, the sdAb includes one, two, or three of the CDRs of BC3-BC14. When multiple CDRs are present they do not need to be from the same sdAb.

BC2 was reported to bind with non-phosphorylated β-catenin at the N-terminus, specifically to residues 15-29, with a dissociation constant of 1.9 nM. The binding site does not interfere with binding of b-catenin with its interaction proteins (e.g., TCF4, Axin, etc.). Overexpression of BC2 in HEK293T does not impact the β-catenin signaling pathway (Traenkle, B. et al., Mol. Cell. Proteomics 2015, 14(3), 707-723).

In embodiments, the targeting moiety comprises a single-domain antibody that specifically binds to EGFR. In embodiments, the single domain antibody that specifically binds to EGFR may be referred to herein as 7D12.

In embodiments, the targeting moiety comprises a single-domain antibody that specifically binds to KRAS. In embodiments, the single domain antibody that specifically binds to KRAS may be referred to herein as KRAS^{VHH}. In embodiments, KRAS^{VHH} may include a N-terminal or C-terminal cysteine (KRAS-Cys).

In embodiments, the targeting moiety comprises a single-domain antibody that specifically binds to HRAS. In embodiments, the single domain antibody that specifically binds to KRAS may be referred to herein as HRAS VHH. In embodiments, KRAS VHH may include a N-terminal or C-terminal cysteine (HRAS VHH-Cys).

In embodiments, the targeting moiety comprises a single-domain antibody that specifically binds to ASC. In embodiments, the single-domain antibody that specifically binds to ASC may be referred to herein as VHH^{CARD} or ACS^{VHH}. In embodiments, ACS^{VHH} may include a N-terminal or C-terminal cysteine (ACS^{VHH}-Cys). In some embodiments, ACS^{VHH} may include a N-terminal or C-terminal LPETG sequence (SEQ ID NO: 73) (ACS^{VHH}-LEPTG VHH-Cys).

VHH^{CARD} is a single-domain antibody that was identified through immunization of an alpaca with human MBP-ASC followed by cloning, screening and isolation from a phage display library. VHH^{CARD} specifically binds to the CARD domain of human ASC and prevents CARD-CARD interaction and polymerization. Expression of VHH^{CARD} in mammalian cell lines effectively inhibits NLRP3/AIM2 and NAIP/NLRC4 inflammasome activation as determined through IL-10 secretion, speck formation, and ASC filament assembly.

In some embodiments, the targeting moiety is a sdAbs that has at least 80%, 85%, 90%, 91%, 92%, 93%, 94%, 95%, 96%, 97%, 98%, or 99% percent similarity and/or percent identity to any one of BC1, BC2, BC2-cys, 7D12, KRAS^{VHH}, KRAS^{VHH}-cys, HRAS^{VHH}, HRAS^{VHH}-cys, ASC^{VHH}, ASC^{VHH}-cys, or ASC^{VHH}-LPETG, as listed in Table 4A. In embodiments, the targeting moiety is an sdAbs that has 80% to 100%, 85% to 100%, 90% to 100%, 95% to 100%, 97% to 100%, 99% to 100%, 90% to 99%, 95% to 99%, or 97% to 99% percent similarity and/or percent identity to any one of BC1, BC2, BC2-cys, 7D12, KRAS^{VHH}, KRAS^{VHH}-cys, HRAS^{VHH}, HRAS^{VHH}-cys, ASC^{VHH}, ASC^{VHH}-cys, or ASC^{VHH}-LPETG, as listed in Table 4A.

In embodiments, the targeting moiety is a sdAb and the sdAb sequence includes a C-terminal and/or N-terminal cysteine to provide a site for conjugation to EEV and/or degradation moiety as described herein. The addition of a C-terminal and/or N-terminal cysteine may be denoted by a -cys (or -Cys) on the N-terminal and/or C-terminal side of the sdAbs. In embodiments, the single domain antibodies listed in Tables 4A-C may further include a C-terminal and/or a N-terminal cysteine.

Amino acid sequences of some examples of single domain antibodies are provided in Tables T4A-C below.

TABLE 4A
Examples of Single Domain Antibodies

| SEQ | sdAb | |
|---|---|---|
| ID | name: | |
| NO: | Fragment | Sequence (N to C) |
| 74 | BC1 | QVQLVESGGGLVQPGGSLTLSCTAS |
| | | GFTLDHYDIGWFRQAPGKEREGVSC |
| | | INNSDDDTYYADSVKGRFTIFMNNA |
| | | KDTVYLQMNSLKPEDTAIYYCNALS |
| | | GLPPYATTYWGQGTQVTVSSKKK |
| 75 | BC2 | QVQLVESGGGLVQPGGSLTLSCTAS |
| | | GFTLDHYDIGWFRQAPGKEREGVSC |
| | | INNSDDDTYYADSVKGRFTIFMNNA |
| | | KDTVYLQMNSLKPEDTAIYYCAEAR |
| | | GCKRGRYEYDFWGQGTQVTVSS |
| 76 | BC2- | QVQLVESGGGLVQPGGSLTLSCTAS |
| | cys | GFTLDHYDIGWFRQAPGKEREGVSC |
| | | INNSDDDTYYADSVKGRFTIFMNNA |
| | | KDTVYLQMNSLKPEDTAIYYCAEAR |
| | | GCKRGRYEYDFWGQGTQVTVSSC |
| 77 | 7D12 | QVKLEESGGGSVQTGGSLRLTCAAS |
| | | GRTSRSYGMGWFRQAPGKEREFVSG |
| | | ISWRGDSTGYADSVKGRFTISRDNA |
| | | KNTVDLQMNSLKPEDTAIYYCAAAA |
| | | GSAWYGTLYEYDYWGQGTQVTVSS |
| 78 | KRAS^{VHH} | EVQLVESGGGSVQTGGSLRLSCAVS |
| | | GNIGSSYCMGWFRQAPGKKREAVAR |
| | | IVRDGATGYADYVKGRFTISRDSAK |
| | | NTLYLQMNRLIPEDTAIYYCAADLP |
| | | PGCLTQAIWNFGYRGQGTLVTVSS |
| 79 | KRAS^{VHH} | EVQLVESGGGSVQTGGSLRLSCAVS |
| | -cys | GNIGSSYCMGWFRQAPGKKREAVAR |
| | | IVRDGATGYADYVKGRFTISRDSAK |
| | | NTLYLQMNRLIPEDTAIYYCAADLP |
| | | PGCLTQAIWNFGYRGQGTLVTVSSC |
| 80 | HRAS^{VHH} | EVQLLESGGGLVQPGGSLRLSCAAS |
| | | GFTFSTFSMNWVRQAPGKGLEWVSY |
| | | ISRTSKTIYYADSVKGRFTISRDNS |
| | | KNTLYLQMNSLRAEDTAVYYCARGR |
| | | FFDYWGQGTLVTVSS |
| 81 | HRAS^{VHH} | EVQLLESGGGLVQPGGSLRLSCAAS |
| | -cys | GFTFSTFSMNWVRQAPGKGLEWVSY |
| | | ISRTSKTIYYADSVKGRFTISRDNS |
| | | KNTLYLQMNSLRAEDTAVYYCARGR |
| | | FFDYWGQGTLVTVSSC |
| 82 | ASC^{VHH} | QLQLVESGGGLVQPGGSLKLSCAAS |
| | | GFTFSRYAMSWYRQAPGKERESVAR |
| | | ISSGGGTIYYADSVKGRFTISREDA |
| | | KNTVYLQMNSLKPEDTAVYYCYVGG |
| | | FWGQGTQVTVSS |
| 83 | ASC^{VHH} | QLQLVESGGGLVQPGGSLKLSCAAS |
| | -cys | GFTFSRYAMSWYRQAPGKERESVAR |
| | | ISSGGGTIYYADSVKGRFTISREDA |
| | | KNTVYLQMNSLKPEDTAVYYCYVGG |
| | | FWGQGTQVTVSSC |
| 84 | ASCVHH- | QLQLVESGGGLVQPGGSLKLSCAAS |
| | LPETG | GFTFSRYAMSWYRQAPGKERESVAR |
| | | ISSGGGTIYYADSVKGRFTISREDA |
| | | KNTVYLQMNSLKPEDTAVYYCYVGG |
| | | FWGQGTQVTVSSLPETG |

TABLE 4B
Examples of Beta catenin antibodies

| | SEQ ID | |
|---|---|---|
| sdAB | NO: | Sequence (N to C) |
| BC3 | 85 | EVQLVQPGGSLRLSCAVSGSISNFN |
| | | AMGWYRQAPGKQRELVATITRGGST |
| | | DYAASVKGRFTISRDSAANTVYLQM |
| | | NSLKPEDTAVYYCNAQRSPLILTTS |
| | | RTHNYWGQGTQVTVSS |
| BC4 | 86 | QVQLVQPGGSLKLSCAASGSIVSIN |
| | | TMAWYRQAPGKQRELIAFITSGGST |
| | | NYADSVKGRFTISRDNAKNTMYLQM |
| | | SSLKPEDTAVYYCNADALYDGLQGL |
| | | TRGYWGQGTRVTVST |
| BC5 | 87 | VQRQESGGGVWQAGGSLRLSCTASG |
| | | AVYNIDVMGWYRQAPGKERELVAGI |
| | | ASDESTSYGDSVKGRFLISMDRAKN |
| | | TVFLKMDNLRPEDTAVYTCRANINR |
| | | YDYWGTGSQVTVSS |
| BC6 | 88 | QVQRQESGGGLVQPGGSLRLSCAAS |
| | | GDIFRVNDMAWYRQAPGKQRELVAT |
| | | ITRGGGVAYADSVKGRFTISRDNSK |
| | | NTVALQMNSLKPDDTAVYYCNADVQ |
| | | VGRNYEYNYWGQGTQVTVSS |
| BC7 | 89 | QVQLVQPGGSLRLSCAASGRDFSGS |
| | | TMGWFRQAPGKERELVAQIAWSGSI |
| | | YTSRAATGRFDISRDNDKNMMWLQM |
| | | NRLKPEDTGLYSCAAGKSRDDYDVW |
| | | GQGTQVTVSS |
| BC8 | 90 | EVQLVQPGGSLRLSCKASGNIFRMV |
| | | DMGWFRQVPGKQRELVARINSDGVA |
| | | SYSDSVRGRFTISGSLAVNTVYLDM |
| | | TNLRSEDTAVYVCNANFDHLIGWNV |
| | | PARNDAWGQGTRVTVAE |
| BC9 | 91 | EVQLVQPGGSLRLSCAASEKIFSIT |
| | | AMGWYRQAPGKQRELVASIASGGTT |
| | | NYADSVKGRFTISRGNAKNTVYLQM |
| | | NSLEAEDTAVYYCHAESEENWLRDY |
| | | WGQGIQVTVSS |
| BC10 | 92 | QVQLVESVGGWESGGFLRLSCQASG |
| | | SVRNINFIGWYRQAPGKQREPVAGI |
| | | TASGRIGYVNSVKGRFTISRDTAKN |
| | | TVYLQMDKLKPEDTGVYLCNAKWLG |
| | | NEGTAYWGQGTQVTVTS |
| BC11 | 93 | QVQLVETGGGLVQPGGSLRLSCVAS |
| | | GFKFDAYAMSWARQAPGKWLEWVSD |
| | | ISWNGVSEYYAESMKGRFTISRDNA |
| | | KNTVYLQMSSLKPEDTAVYYCAKQA |
| | | RPQEVGVPTFQVTGYGMDYWGKGAL |
| | | VTASS |
| BC12 | 94 | EVQVQESGGGLVQPGGSLRLSCAAS |
| | | GSFYSIVAMGWYRQAPGQQRELVAT |
| | | VTSGGSGGSVTNYADSVKGRFTISR |
| | | DNAKNTVDLQMNSLKPDDTAVYSCW |
| | | ARVRVTPERFFSYWDRGTQVTVAS |
| BC13 | 95 | QVQLVETGGGLVAPGGSLTLSCVAS |
| | | GSIESIYSMGWYRQAPGKERELVAD |
| | | TTAGNTPHYGESVRGRFTISSISRD |
| | | SAKNTVYLQMSDLKPDDTAVYYCAM |
| | | GISHNWYAPSIYWGRGTQVTVSS |
| BC14 | 96 | GGALAQPGGSLTLSCAVSGFTFDYD |
| | | AAELRDYVLGWIRQVPGKEPEGVSC |
| | | SSRDGTAYYSDSVKGRFTIYRDNQK |
| | | NTVYLQMNSLKPDDTAVYYCAAHDL |
| | | IVYYNDFPLCSTYDYDYWGQGIQVT |
| | | VSS |

TABLE 4C
Beta catenin sdAb CDRs

| | SEQ | CDR1 | SEQ | CDR2 | SEQ | CDR3 |
|---|---|---|---|---|---|---|
| | ID | (N to | ID | (N to | ID | (N to |
| sdAB | NO: | C) | NO: | C) | NO: | C) |
| BC3 | 97 | GSISN | 109 | ITRGG | 121 | NAQRS |
| | | FNA | | ST | | PLILT |
| | | | | | | TSRTH |
| | | | | | | NY |
| BC4 | 98 | GSIVS | 110 | ITSGG | 122 | NADAL |
| | | INT | | ST | | YDGLQ |
| | | | | | | GLTRG |
| | | | | | | Y |
| BC5 | 99 | GAVYN | ill | IASDE | 123 | RANIN |
| | | IDV | | ST | | RYDY |
| BC6 | 100 | GDIFR | 112 | ITRGG | 124 | NADVQ |
| | | VND | | GV | | VGRNY |
| | | | | | | EYNY |
| BC7 | 101 | GRDFS | 113 | IAWSG | 125 | AAGKS |
| | | GST | | SI | | RDDYD |
| | | | | | | V |
| BC8 | 102 | GNIFR | 114 | INSDG | 126 | NANED |
| | | MVD | | VA | | HLIGW |
| | | | | | | NVPAR |
| | | | | | | NDA |
| BC9 | 103 | EKIES | 115 | IASGG | 127 | HAESE |
| | | ITA | | TT | | ENWLR |
| | | | | | | DY |
| BC10 | 104 | GSVRN | 116 | ITASG | 128 | NAKWL |
| | | INF | | RI | | GNEGT |
| | | | | | | AY |
| BC11 | 105 | GFKFD | 117 | ISWNG | 129 | AKQAR |
| | | AYA | | VSE | | PQEVG |
| | | | | | | VPTFQ |
| | | | | | | VTGYG |
| | | | | | | MDY |
| BC12 | 106 | GSFYS | 118 | VTSGG | 130 | YSCWA |
| | | IVA | | SGG | | RVRVT |
| | | | | | | PERFF |
| | | | | | | SY |
| BC13 | 107 | GSIFS | 119 | TTAGN | 131 | YYCAM |
| | | IYS | | TP | | GISHN |
| | | | | | | WYAPS |
| | | | | | | IY |
| BC14 | 108 | AAELR | 120 | SSRDG | 132 | AAHDL |
| | | DYV | | TA | | IVYYN |
| | | | | | | DFPLC |
| | | | | | | STYDY |
| | | | | | | DY |

Antibody Mimetics

In embodiments, the targeting moiety comprises an antibody mimetic. An antibody mimetic refers to a polypeptide that can specifically bind an antigen but is not structurally related to an antibody. They are usually artificial peptides or proteins with a molar mass of about 3 to 20 kD. In embodiments, the antibody mimetic is a monobody, an affibody, an affilin, an affimer, and affitin, an alphabody, an anticalin, an avimer, a DARPin, a fynomer, a Kunitz domain peptide, or combinations thereof.

In embodiments, the targeting moieties comprises “monobodies”. The term “monobody” (Mb) refers to a synthetic binding protein constructed using a fibronectin type III domain (FN3) as a molecular scaffold.

In embodiments, the antigen binding constructs described herein are “designed ankryin repeats” or “DARPins”. DARPins are derived from natural ankyrin proteins comprised of at least three repeat motifs proteins, and usually comprise of four or five repeats.

In embodiments, the present disclosure provides a DARPin that specifically binds to ASC or a mutant thereof. In embodiments, the DARPin that specifically binds to ASC may be referred to herein as K19. In embodiments, the DARPin that specifically binds to ASC may be referred to herein as K13. K19 and K13 have been previously described (Bery et al., Nature Communications (2019), 10, 2607).

In embodiments, the present disclosure provides a monobody that specifically binds to MDM2, or a mutant thereof. In embodiments, the monobody that specifically binds to ASC may be referred to herein as MOP3. In embodiments, the monobody that specifically binds to ASC may be referred to herein as MOP3+. In embodiments, the monobody that specifically binds to ASC may be referred to herein as MOP4.

In embodiments, the targeting moiety is a sdAbs that has at least 80%, 85%, 90%, 91%, 92S, 9300 9400 9500 96E, 9700 98E, or 990 percent similarity and/or percent identity to any one of K19, K13, MOP3+, MOP, MOP4, as listed in Table 5. In embodiments, the targeting moiety is an sdAbs that has 80% to 100%, 85% to 100%, 90% to 100%, 95% to 100%, 97% to 100%, 99% to 100%, 90% to 99%, 95% to 99%, or 97% to 99% percent similarity and/or percent identity to any one of K19, K13, MOP3+, MOP, MOP4, as listed in Table 5.

In embodiments, the targeting moiety is an antibody mimetic and the antibody mimetic sequence includes a C-terminal and/or N-terminal cysteine to provide a site for conjugation to EEV, additional targeting moiety, and/or degradation moiety as described herein. The addition of a C-terminal and/or N-terminal cysteine may be denoted by a -cys (or -Cys) on the N-terminal and/or C-terminal side of the antibody mimetic name. In embodiments, the antibody mimetics K19, K13, MOP3+, MOP, or MOP4 include a C-terminal and/or a N-terminal cysteine.

Amino acid sequences of some examples of antibody mimetics are provided in Table 5. below.

TABLE 5
Examples of Some Antibody Mimetics

| SEQ ID | Antibody | Sequence |
|---|---|---|
| NO: | mimetic | (N to C) |
| 133 | K19 | MDLGKKLLEAARAGQDDEVRILMAN |
| | | GADVNASDRWGWTPLHLAAWWGHLE |
| | | IVEVLLKRGADVSAADLHGQSPLHL |
| | | AAMVGHLEIVEVLLKYGADVNAKDT |
| | | MGATPLHLAARSGHLEIVEELLKNG |
| | | ADMNAQDKFGKTTFDISTDNGNEDL |
| | | AEILQKL |
| 134 | K13 | MDLGKKLLEAARAGQDDEVRILMAN |
| | | GADVNASDRWGWTPLHLAAWWGHLE |
| | | IVEVLLKHGADVNAADLHGQTPLHL |
| | | AAMVGHLEIVEVLLKYGADVNAKDT |
| | | MGATPLHLAAQSGHLEIVEVLLKNG |
| | | ADVNAQDKEGKTAFDISIDNGNEDL |
| | | AEILQKL |
| 135 | MOP3 | MVSDVPRKLEVVAATPTSLLISWDA |
| | | PAVTVRYYRITYGETSGGGSFAEYW |
| | | ALLSGGGSVQEFTVPGSKSTATISG |
| | | LKPGVDYTITVYAVTSGGGSFAEYW |
| | | ALLSGGGSPISINYRTALESD |
| 136 | MOP3 | MVSDVPRKLEVVAATPTSLLISWDA |
| | | PAVTVRYYRITYGETGGNSPVQEFT |
| | | VPGSKSTATISGLKPGVDYTITVYA |
| | | VTSGGGSFAEYWALLSGGGSPISIN |
| | | YRTALE |
| 137 | MOP4 | VSDVPRKLEWAATPTSLLISWDAPA |
| | | VTVRYYRITYGETSGGGSFAEYWAL |
| | | LSGGGSVQEFTVPGSKSTATISGLK |
| | | PGVDYTITVYAVTSGGGSFAEYWAL |
| | | LSGGGSPISINYRTALE |

Additional Targeting Moieties

In embodiments, the degradation compound includes additional targeting moieties. In embodiments, the degradation compound includes a second targeting moiety. The second targeting moiety may be any antibody, antigen binding fragment, or antibody mimetic as described elsewhere herein. In embodiments, where the degradation compound includes two or more targeting moieties, the first targeting moiety binds to the target protein (i.e., the protein being targeted for degradation). In embodiments, the second targeting moiety binds a target antigen that is not the target protein. In embodiments, the second targeting moiety is an extracellular targeting moiety. For example, the second targeting moiety may bind an antigen that is displayed on the surface of a target cell. In embodiments, the second targeting moiety is an intracellular targeting moiety that binds to an antigen that is within a cell.

In embodiments, the degradation compound targets a particular cell type. In embodiments, the degradation compound targets a cell that displays particular markers. In embodiments, cells of the particular type or displaying the particular markers are cells that contain the target protein. In embodiments, the second targeting moiety targets the degradation compound to the particular cell type or the cell displaying the particular markers. In embodiments, the second targeting moiety binds to a cell antigen displayed on a target cell. In embodiments, binding to antigen on the surface of the cell triggers target cell specific endocytosis by which the degradation compound enters cells with the specific surface receptor that is bound by the second targeting moiety. Cell specific endocytosis may be advantageous as it may allow for the degradation compound to enter specific cells associated with, for example, a particular disease. Such targeting may, thus, be beneficial for treating the particular disease and may limit effects on cells that do not substantially contribute to the disease.

In embodiments, where the degradation construct of the degradation compound includes a first targeting moiety and a second targeting moiety, the targeting moieties can collectively be referred to as bi-specific. That is, the targeting moieties of the degradation compound may be bi-specific constructs. The bi-specific construct may include a targeting moiety that binds to the target protein (the first targeting moiety) and a second targeting moiety that binds to a second antigen (the second targeting moiety).

In embodiments, the degradation compound includes a second targeting moiety and a third targeting moiety. In embodiments, the second targeting moiety and the third targeting moiety bind to different cell surface antigens. In embodiments, the second targeting moiety and the third targeting moiety bind to different surface antigens on the same cell. In embodiments, the second targeting moiety and the third targeting moiety bind to different cell surface antigens on different cells.

Cancer cells can co-express multiple target antigens, and therefore a degradation compound comprising second and third targeting moieties can increase the number of target cells to which the bi-specific construct can bind. Additionally, a degradation compound comprising second and third targeting moieties can be used to bring two different cell types in close proximity to one another, such as an effector cell (e.g., a T cell, NK cell, NKT cell, neutrophil, macrophage, etc.) and a target cell (e.g., a tumor) cell. In such embodiments, the bi-specific construct can direct the effector cell function (e.g., cell lysis) to the target cell.

The bi-specific constructs described herein can be of any suitable format. In embodiments, the bi-specific constructs allow binding to the target protein and a second target antigen. In embodiments, the bi-specific antigen-binding construct is a diabody or DVD-Ig. In embodiments, the bi-specific antigen-binding construct is a fusion or linkage of two independent antigen-binding moieties. In embodiments, the two independent antigen-binding moieties are the same type (i.e., two scFvs, two nanobodies, two antibody mimetics, etc.). In embodiments, the two independent antigen-binding moieties are different types (i.e., an scFv and a nanobody, an scFv and an antibody mimetic, a nanobody and an antibody mimetic, etc.).

In embodiments, the present disclosure provides a bi-specific construct comprising a first targeting moiety that specifically binds to KRAS and a second targeting moiety that specifically binds to EGFR. In some embodiments, the first targeting moiety the antibody mimetic K19 and the second targeting moiety is the single domain antibody 7D12 that specifically binds to EGFR.

In embodiments, the first targeting moiety is K19 or a sequence that has 80% to 100%, 85% to 100%, 90% to 100%, 95% to 100%, 97% to 100%, 99% to 100%, 90% to 99%, 95% to 99%, or 97% to 99% percent similarity and/or percent identity to K19. In embodiments, the second targeting moiety is 7D12 or a sequence that has 80% to 100%, 85% to 100%, 90% to 100%, 95% to 100%, 97% to 100%, 99% to 100%, 90% to 99%, 95% to 99%, or 97% to 99% percent similarity and/or percent identity to 7D12.

In embodiments, the degradation construct includes a first targeting moiety and a second targeting moiety that are joined via a linker. Any suitable linker may be used such as those in Table 10 and described elsewhere.

In embodiments, the bispecific construct sequence includes a C-terminal and/or N-terminal cysteine to provide a site for conjugation an EEV and/or degradation moiety as described herein.

The addition of a C-terminal and/or N-terminal cysteine may be denoted by a -cys (or -Cys) on the N-terminal and/or C-terminal side of the sdAbs.

Amino acid sequences of some examples of bi-specific constructs that have targeting moieties binding to KRAS and EGFR are provided in Table 6 below. Construct details are written N to C. Although no linker is not explicitly included in the construct details, a linker may be present between targeting moieties.

TABLE 6
Some Examples of Bispecific Constructs

| SEQ | Construct | |
|---|---|---|
| ID | (N to | |
| NO: | C) | Sequence (N to C) |
| 138 | Cys- | CQVKLEESGGGSVQTGGSLRLTCAASGRTSRSYGMGW |
| | 7D12- | FRQAPGKEREFVSGISWRGDSTGYADSVKGRFTISRD |
| | K19 | NAKNTVDLQMNSLKPEDTAIYYCAAAAGSAWYGTLYE |
| | | YDYWGQGTQVTVSSGGGGSGGGGSDLGKKLLEAARAG |
| | | QDDEVRILMANGADVNASDRWGWTPLHLAAWWGHLEI |
| | | VEVLLKRGADVSAADLHGQSPLHLAAMVGHLEIVEVL |
| | | LKYGADVNAKDTMGATPLHLAARSGHLEIVEELLKNG |
| | | ADMNAQDKFGKTTFDISTDNGNEDLAEILQKLGGGGS |
| | | TLPVYTLKERCLQVVRSLVKPENYRRLDIVRSLYEDL |
| | | EDHPNVQKDLERLTQERIAHQRMGDLKNGADMNAQDK |
| | | FGKTTFDISTDNGNEDLAEILQKL |
| 139 | 7D12- | QVKLEESGGGSVQTGGSLRLTCAASGRTSRSYGMGWF |
| | K19- | RQAPGKEREFVSGISWRGDSTGYADSVKGRFTISRDN |
| | Cys | AKNTVDLQMNSLKPEDTAIYYCAAAAGSAWYGTLYEY |
| | | DYWGQGTQVTVSSGGGGSGGGGSDLGKKLLEAARAGQ |
| | | DDEVRILMANGADVNASDRWGWTPLHLAAWWGHLEIV |
| | | EVLLKRGADVSAADLHGQSPLHLAAMVGHLEIVEVLL |
| | | KYGADVNAKDTMGATPLHLAARSGHLEIVEELLKNGA |
| | | DMNAQDKFGKTTFDISTDNGNEDLAEILQKLC |
| 140 | BC2- | QVQLVESGGGLVQPGGSLTLSCTASGFTLDHYDIGWF |
| | 7D12- | RQAPGKEREGVSCIANSDDDTYYADSVKGRFTIFMNN |
| | cys | AKDTVYLQMNSLKPEDTAIYYCAEARGCKRGRYEYDF |
| | | WGQGTQVTVSSAAAQVKLEESGGGSVQTGGSLRLTCA |
| | | ASGRTSRSYGMGWFRQAPGKEREFVSGISWRGDSTGY |
| | | ADSVKGRFTISRDNAKNTVDLQMNSLKPEDTAIYYCA |
| | | AAAGSAWYGTLYEYDYWGQGTQVTVSSC |

In embodiments, the bi-specific antigen-binding construct comprises a sequence has at least 80%, 85%, 90%, 91%, 92%, 93%, 94%, 95%, 96%, 97%, 98%, or 99% percent similarity and/or percent identity to any one of Cys-7D12-K19, 7D12-K19-Cys, and BC2-7D12-cys as listed in Table 6. In embodiments, the targeting moiety is an sdAbs that has 80% to 100%, 85% to 100%, 90% to 100%, 95% to 100%, 97% to 100%, 99% to 100%, 90% to 99%, 95% to 99%, or 97% to 99% percent similarity and/or percent identity to any one of Cys-7D12-K19, 7D12-K19-Cys, and BC2-7D12-cys as listed in Table 6.

In embodiments, the second targeting moiety may bind to an infectious agent, for example, a bacterial antigen, a viral antigen, a fungal antigen, an algal antigen, or a parasitic antigen. In embodiments, the antigen (e.g., the second antigen) is a protein, polysaccharide, lipid, glycolipid, or combinations thereof that is on the surface or within a cell. In embodiments, the target antigen is an antigen of an infectious agent that is comprised within a synthetic system, for example a target antigen affixed to a particle or particulate matter (e.g., polystyrene beads), for example, that might be used for bioterrorism.

In embodiments, the infectious agent is a Gram-negative bacterium. In embodiments, the Gram-negative bacterium is of a genus selected from the group consisting of Escherichia, Proteus, Salmonella, Klebsiella, Providencia, Enterobacter, Burkholderia, Pseudomonas, Acinetobacter, Aeromonas, Haemophilus, Yersinia, Neisseria, Erwinia, Rhodopseudomonas, Ehrlichia, and Burkholderia. In embodiments, the infectious agent is a Gram-positive bacterium. In embodiments, the Gram-positive bacterium is of a genus selected from Lactobacillus, Azorhizobium, Streptococcus, Pediococcus, Photobacterium, Bacillus, Enterococcus, Staphylococcus, Clostridium, Butyrivibrio, Sphingomonas, Rhodococcus and Streptomyces. In embodiments, the infectious agent is an acid-fast bacteria of the Mycobacterium genus, such as Mycobacterium tuberculosis, Mycobacterium bovis, Mycobacterium avium and Mycobacterium leprae. In embodiments, the infectious agent is of the genus Nocardia. In embodiments, the infectious agent is selected from Nocardia asteroides, Nocardia brasiliensis and Nocardia caviae.

In embodiments, the infectious agent is a fungus. In embodiments, the fungus is from the genus Mucor. In embodiments, the fungus is from the genus Crytococcus. In embodiments, the fungus is from the genus Candida. In embodiments, the fungus is selected from any one of Mucor racemosus, Candida albicans, Crytococcus neoformans, or Aspergillus fumingatus.

In embodiments, the infectious agent is a protozoa. In embodiments, the protozoa is of the genus Plasmodium (e.g., P. falciparum, P. vivax, P. ovale, or P. malariae). In embodiments, the protozoa causes malaria.

In embodiments, the infectious agent incudes Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia.

In embodiments, the infectious agent is a parasite. In embodiments, the parasite is Cryptosporidium. In embodiments, the parasite is an endoparasite. In embodiments, the endoparasite is heartworm, tapeworm, or flatworm. In embodiments, the parasite is an epiparasite.

In embodiments, the parasite causes a disease selected from acanthamoebiasis, babesiosis, balantidiasis, blastocystosis, coccidiosis, amoebiasis, giardiasis, isosporiasis, cystosporiasis, leishmaniasis, primary amoebic meningoencephalitis, malaria, rhinosporidiosis, toxoplasmosis, trichomoniasis, trypanomiasis, Chagas disease, or scabies.

In embodiments, the infectious agent is a virus. Non-limiting examples of viruses include sudden acute respiratory coronavirus 2 (SARS-CoV-2), sudden acute respiratory coronavirus (SARS-CoV), respiratory syncytial virus (RSV), Middle East Respiratory virus (MERS), influenza virus (including influenza A, B, and C), parainfluenza virus, Hepatitis C virus, adenovirus; human rhinovirus; coronavirus; norovirus, Dengue virus, West Nile virus, Ebola virus, Hepatitis B, Human immunodeficiency virus (HIV), herpes simplex, Herpes zoster, and Lassa virus.

Many tumors and cancers have viral genome present in the tumor or cancer cells. For example, Epstein-Barr Virus (EBV) is associated with a number of mammalian malignancies. In embodiments, the constructs disclosed herein can be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells.

Infectious agents can include pathogens that infect humans and pathogens that infect affect non-human animals, including, but not limited to, livestock such as swine, ruminants (e.g., cattle, goats, sheep), ungulates (e.g., horses), poultry, and the like.

Some examples of antibodies that bind to infectious agents are provided below in Table 7.

Any of these antibodies, or antigen binding fragments derived therefrom, can be utilized as the targeting moiety and/or second targeting moiety. Each publication in Table 7 is incorporated by reference in its entirety for all purposes.

TABLE 7
Some Examples of Infectious Agent Antigen-binding moieties

| Infectious | |
|---|---|
| agent target | Reference |
| Adenovirus | U.S. Pat. No. 4,487,829 |
| Actinomyces | EP1556499A4 |
| Aspergillus | Trazimab-Takis Biotech |
| Bacillus | Obiltoxaximab-Elusys; Thravixa-Emergent; Valortim-PharmAthene; |
| | Raxibacumab-HSG/GSK; |
| Borrelia | U.S. Pat. No. 5,856,447 |
| Brucella | CN105218668A |
| Campylobacter | U.S. Pat. No. 6,551,599B2 |
| Candida | WO2016142660A1 |
| Chlamydia | US20060073531A1 |
| Chlamydophila | CN104130326A |
| Clostridium | XOMA 3ab-XOMA/NIAID; NTM-1632-NIAID; Actoxumab-Merck; |
| | Bezlotoxumab-Merck; |
| Corynebacterium | Production of Monoclonal Antibodies to Corynebacterium sepedonicum. |
| | S. H. De Boer, Agriculture Canada Research Station, Vancouver, B.C., |
| | Canada, V6T 1X2 |
| Cryptosporidium | U.S. Pat. No. 6,777,222B1 |
| Crytococcus | Phase I evaluation of the safety and pharmacokinetics of murine-derived |
| | anticryptococcal antibody 18B7 in subjects with treated cryptococcal |
| | meningitis, DOI: 10.1128/AAC.49.3.952-958.2005 |
| Dengui virus | U.S. Pat. No. 7,622,113B2 |
| Ebola | Ansuvimab (Ebanga); Inmazeb-Regeneron; |
| Ehrlichia | US20170204168A1 |
| Eimeria | EP0135073A3 |
| Entamoeba | CA2472308A1 |
| Enterobacter | Extended-spectrum antibodies protective against |
| | carbapenemase-producing Enterobacteriaceae, J Antimicrob Chemother |
| | 2016; 71: 927-935 (doi: 10.1093/jac/dkv448) |
| Enterococcus | WO2015101438A1 |
| Epstein-barr | WO2015199618A1; US20110135642A1 |
| virus | |
| Escherichia | Edobacumab-XOMA; Nebacumab-Centocor; T88-Chiron; Shiga toxin |
| | MAbs, cStx1 and -2-Taro (Thallion) |
| Giardia | CA2344589A1 |
| Haemophilus | U.S. Pat. No. 5,013,664 |
| Helicobacter | WO2016000022A1 |
| Hepatitis B | U.S. Pat. No. 5,204,095; WO2017059878A1 |
| Hepatitis C | U.S. Pat. No. 7,314,919B2; U.S. Pat. No. 5,595,868A |
| Herpes simplex | U.S. Pat. No. 5,646,041; WO2010087813A1 |
| virus | |
| HIV | Ibalizumab-Genetech (Tanox); U.S. Pat. No. 1,039,2433B2; |
| | U.S. Pat. No. 1,042,1803B2; U.S. Pat. No. 1,065,4943B2 |
| HPV (Human | U.S. Pat. No. 8,865,162B2; CN103130890B |
| papillomavirus) | |
| Influenza | U.S. Pat. No. 9,718,874B2; U.S. Pat. No. 9,718,873B2; U.S. Pat. No. 9,340,603B2 |
| Klebsiella | US20190062411A1; |
| | Endotoxin neutralization by an O-antigen specific monoclonal antibody: |
| | a potential novel therapeutic approach against Klebsiella pneumoniae ST258. |
| | Virulence https://doi.org/10.1080/21505594.2017.1279778.; |
| | Antibody-based immunotherapy to treat and prevent infection with |
| | hypervirulent Klebsiella pneumoniae. |
| | Clin Vaccine Immunol 24: e00456-16. |
| | https://doi.org/10.1128/CVI.00456-16. |
| Lactobacillus | EP1556499A4 |
| Lassa virus | Antibody therapy for Lassa fever, |
| | https://doi.org/10.1016/j.coviro.2019.07.003 |
| Legionella | U.S. Pat. No. 4,931,547 |
| Leishmania | EP0197062B1 |
| Leptospira | WO2014188763A1 |
| Listeria | U.S. Pat. No. 7,390,489B2 |
| MERS-CoV | U.S. Pat. No. 1,040,6222B2 |
| Mucorales | U.S. Pat. No. 9,279,002B2 |
| (Rhizopus | |
| oryzae) | |
| Mycobacterium | US20010007660A1 |
| Mycoplasma | U.S. Pat. No. 4,945,041 |
| Neisseria | WO/1998/008874 |
| Norovirus | US20140017257 A1 |
| Parainfluenza | Protective antibodies against human parainfluenza virus type 3 infection, |
| (PTV) | https://doi.org/10.1080/19420862.2021.1912884 |
| Pediococcus | U.S. Pat. No. 6,395,500B1 |
| Plasmodium | A human monoclonal antibody prevents malaria infection by targeting a |
| | new site of vulnerability on the parasite, DOT: 10.1038/nm.4512 |
| Proteus | U.S. Pat. No. 4,772,464 |
| Pseudomonas | U.S. Pat. No. 4,834,976; EP0421382B1 |
| Pseudomonas | Aerucin-Aridis; Panobacumab-Aridis; KB001-KaloBios; MEDI3902- |
| | MedImmune |
| Rhinovirus | Antibody-Mediated Neutralization of Human Rhinovirus 14 Explored by |
| | Means of Cryoelectron Microscopy and X-Ray Crystallography of Virus- |
| | Fab Complexes, J Virol. 1998 Jun; 72(6): 4610-4622. |
| RSV | Synagis-SOBI (AstraZeneca); U.S. Pat. No. 7,364,742B2; |
| | U.S. Pat. No. 1,008,1671B2; U.S. Pat. No. 9,273,122B2; |
| | U.S. Pat. No. 1,072,3786B2 |
| Salmonella | U.S. Pat. No. 1,054,4208B2 |
| SARS-CoV | U.S. Pat. No. 7,622,112B2 |
| SARS-CoV-2 | sotrovimab-GSK; bamlanivimab-Eli Lilly |
| Staphylococcus | U.S. Pat. No. 7,531,633B2; Salvecin-Aridis (Kenta); ASN100-Arsanis; |
| | Tefibazumab-Bristol-Myers |
| | Squibb(Inhibitex); MEDI4893-Astra Zeneca (MedImmune); 514G3- |
| | Xbiotech; Pagibaximab-Biosynexus; Aurograb-NeuTec |
| | Pharma/Novartis; |
| Streptococci | WO 2003/059259; WO2005047309A2 |
| Streptomyces | WO2002027322A2 |
| Toxoplasmosis | US20020058288A1 |
| Treponema | U.S. Pat. No. 5,514,553 |
| Trichomoniasis | CA2482334C |
| Trypanosoma | U.S. Pat. No. 9,482,667B2 |
| Varicella-zoster | U.S. Pat. No. 5,506,132 |
| virus | |
| Vibrio | CN102659942B; WO2015122598A1 |
| West Nile | Omr-IgG-am-NIH |
| Virus | |
| Yersinia | WO2005047309A2 |

### Degradation Moieties

The degradation constructs of the degradation compounds of the present disclosure include a degradation moiety. The degradation moiety may mediate proteasomal and/or autophagy mediated degradation of the target protein. The derogation moiety may mediate degradation through ubiquitination of the target antigen leading to degradation of the target antigen by proteasomal degradation or autophagy. As used herein, the term “ubiquitination” refers to the attachment of the protein ubiquitin to lysine residues of other molecules. Ubiquitination of a peptide or protein can act as a signal for its rapid cellular degradation, and for targeting to the proteasome complex. The degradation moiety can mediate degradation of the target protein either through direct action of the degradation moiety itself or indirectly through the recruitment of endogenous cellular proteins that mediate degradation of the target protein.

The degradation moiety may be an E3 ligase or an active fragment thereof, or an E3 ligase recruiting domain. As used herein “active fragment” or “active fragment thereof” refers to a fragment of a polypeptide that retains the function of the polypeptide, such as, for example, an E3 ligase. As used herein an “E3 ligase or an active fragment thereof” includes E3 ligases and E3 ligase accessory proteins or active fragments thereof. The degradation moiety may be any E3 ligase or an active fragment thereof such as those listed in Table 1A or any E3 ligase accessory protein such as those listed in Table 1B. The degradation moiety may function to recruit any E3 ligase, such as those listed in Table 1A. The degradation moiety may function to recruit any E3 ligase accessory protein such as those listed in Table 1B.

E3 Ligase or an Active Fragment Thereof

In embodiments, the degradation moiety includes an E3 ligase or an active fragment thereof. E3 ligases and active fragments thereof, mediate degradation of a target antigen through direct action as E3 ligases or active fragments thereof or through acting as an accessory protein or active fragment thereof for a E3 ligase complex. The E3 ligase or fragment thereof is capable of ubiquitinating a substrate. In embodiments, the E3 ligase or fragment thereof comprises a U-box motif. In embodiments, the E3 ligase or fragment thereof comprises a ligase that includes RING domain, a HECT domain, or a Ubox domain. In embodiments, the E3 ligase or fragment thereof participates in larger E3 ligase complexes such a cullin-RING ligase complex (e.g., SCF or anaphase complex).

In embodiments, the E3 ligase or active fragment thereof is a von Hippel-Lindau (VHL, UniProt Ref #: P40337) E3 ubiquitin ligase; a Cereblon (CRBN, UniProt Ref #: 96SW2) E3 ubiquitin ligase; a Tripartite motif-containing protein 21 (TRIM21, UniProt Ref #: P19747) E3 ubiquitin ligase; and a suppressor of cytokine signaling 1 (SOCS1, UniProt Ref #: 015524) E3 ubiquitin ligase. Table 8 shows some examples of E3 ligases and active fragments thereof that may be used as the degradation moiety

In embodiments, the E3 ligase or active fragment thereof is VHL or an active fragment derived from VHL. In embodiments, VHL or an active fragment of VHL includes the Y185F mutation. In embodiments, the active fragment of VHL is VHL peptide 1a (VHLpep1a(152-213) or VHL peptide 1b (VHLpep1b(152-213; Y185F). VHLpep1a includes amino acids 152-213 of VHL. VHLpep1b includes amino acids 152-213 of VHL and the Y185F mutation. In embodiments, the active fragment of VHL is VHL peptide 2a (VHLpep2a(157-194) or VHL peptide 2b (VHLpep2b(157-194; Y185F). VHLpep2a includes amino acids 157-194 of VHL. VHLpep2b includes amino acids 157-194 of VHL and the Y185F mutation. In embodiments, the active fragment of VHL is VHL peptide 3a (VHLpep3a(113-213) or VHL peptide 3b (VHLpep3b(113-213; Y185F). VHLpep3a includes amino acids 113-213 of VHL. VHLpep3b includes amino acids 113-213 of VHL and the Y185F mutation. In embodiments, the active fragment of VHL is VHL peptide 4a (VHLpep4a(103-213) or VHL peptide 4b (VHLpep4b(110-213; Y185F). VHLpep4a includes amino acids 110-213 of VHL. VHLpep4b includes amino acids 113-213 of VHL and the Y185F mutation.

In embodiments, the E3 ligase or active fragment thereof is TRIM21 or derived from TRIM21. In embodiments, the active fragment of TRIM21 is TRIM21 peptide (TRIM21pep (1-277). TRIM21pep includes amino acids 1-277 of TRIM21.

In embodiments, the E3 ligase or active fragment thereof is SOCS1 or derived from SOCS1. In embodiments, the active fragment of SCS1 is SOCS peptide (SOCSpep (170-211). SOCSpep includes amino acids 170-211 of SOCS1.

In embodiments, the E3 ligase or active fragment thereof is CRB or derived from CRB.

In embodiments, the E3 ligase or active fragment thereof comprises a sequence that has at least 80%, 85%, 90%, 91%, 92%, 93%, 94%, 95%, 96%, 97%, 98%, or 99% percent similarity and/or percent identity to any one of VHL (full), VHLpep1a (VHL152-213), VHLpep1b (VHL(157-194(Y185F)), VHLpep2a (VHL(157-194), VHLpep2b (VHL(157-194(Y185F)), VHLpep3a (VHL(113-213), VHLpep3b (VHL(113-213(Y185F)), VHLpep4a (VHL(110-213), VHLpep4b (VHL(110-213(Y185F)), VHLpep5a (VHL(138-213)), VHLpep5b (VHL(138-213 (Y185F)), VHLpep6a (VHL(54-213)), VHLpep6b (VHL(54-213 (Y185F)), TRIM21 (full), TRIM21pep (TRIM21(1-277)), SOCS1 (full), SOCS1pep (SOCS1(170-211)) CRB (full), as listed in Table 8. In embodiments, the E3 ligase or active fragment thereof comprises 80% to 100%, 85% to 100%, 90% to 100%, 95% to 100%, 97% to 100%, 99% to 100%, 90% to 100%, 95% to 99%, or 97% to 99% percent similarity and/or percent identity to any one of VHL (full), VHLpep1a (VHL152-213), VHLpep1b (VHL(157-194(Y185F)), VHLpep2a (VHL(157-194), VHLpep2b (VHL(157-194(Y185F)), VHLpep3a (VHL(113-213), VHLpep3b (VHL(113-213(Y185F)), VHLpep4a (VHL(110-213), VHLpep4b (VHL(110-213(Y185F)), VHLpep5a (VHL(138-213)), VHLpep5b (VHL(138-213 (Y185F)), VHLpep6a (VHL(54-213)), VHLpep6b (VHL(54-213 (Y185F)), TRIM21 (full), TRIM21pep (TRIM21(1-277)), SOCS1 (full), SOCS1pep (SOCS1(170-211)) CRB (full), as listed in Table 8.

In embodiments, the E3 ligase or active fragment thereof includes a C-terminal and/or N-terminal cysteine to provide a site for conjugation an EEV and/or targeting moiety as described herein. The addition of a C-terminal and/or N-terminal cysteine may be denoted by a -cys (or -Cys) on the N-terminal and/or C-terminal side of the E3 ligase or active fragment thereof. In embodiments, degradation moieties listed in Table 8 may further include a C-terminal and/or a N-terminal cysteine.

TABLE 8
Degradation moieties

| SEQ | E3 ligase or | |
|---|---|---|
| ID | active fragment | |
| NO: | thereof | Sequence (N to C) |
| 141 | VHL (full) | MPRRAENWDEAEVGAEEAGVEEYGPEEDGGEESGAEE |
| | | SGPEESGPEELGAEEEMEAGRPRPVLRSVNSREPSQV |
| | | IFCNRSPRVVLPVWLNFDGEPQPYPTLPPGTGRRIHS |
| | | YRGHLWLFRDAGTHDGLLVNQTELFVPSLNVDGQPIF |
| | | ANITLPVYTLKERCLQVVRSLVKPENYRRLDIVRSLY |
| | | EDLEDHPNVQKDLERLTQERIAHQRMGD |
| 142 | VHLpep1a | TLPVYTLKERCLQVVRSLVKPENYRRLDIVRSLYEDL |
| | (VHL152-213) | EDHPNVQKDLERLTQERIAHQRMGD |
| 143 | VHLpep1b | TLPVYTLKERCLQVVRSLVKPENYRRLDIVRSLFEDL |
| | (VHL(157- | EDHPNVQKDLERLTQERIAHQRMGD |
| | 194(Y185F)) | |
| 144 | VHLpep2a | TLKERCLQVVRSLVKPENYRRLDIVRSLYEDLEDHPN |
| | (VHL(157-194) | V |
| 145 | VHLpep2b | TLKERCLQVVRSLVKPENYRRLDIVRSLFEDLEDHPN |
| | (VHL(157- | V |
| | 194(Y185F)) | |
| 146 | VHLpep3a | RGHLWLFRDAGTHDGLLVNQTELFVPSLNVDGQPIFA |
| | (VHL(113-213) | NITLPVYTLKERCLQVVRSLVKPENYRRLDIVRSLYE |
| | | DLEDHPNVQKDLERLTQERIAHQRMGD |
| 147 | VHLpep3b | RGHLWLFRDAGTHDGLLVNQTELFVPSLNVDGQPIFA |
| | (VHL(113- | NITLPVYTLKERCLQVVRSLVKPENYRRLDIVRSLFD |
| | 213(Y185F)) | LEDHPNVQKDLERLTQERIAHQRMGD |
| 148 | VHLpep4a | HSYRGHLWLFRDAGTHDGLLVNQTELFVPSLNVDGQP |
| | (VHL(110-213) | IFANITLPVYTLKERCLQVVRSLVKPENYRRLDI |
| | | VRSLYEDLEDHPNVQKDLERLTQERIAHQRMGD |
| 149 | VHLpep4b | HSYRGHLWLFRDAGTHDGLLVNQTELFVPSLNVDGQP |
| | (VHL(110- | IFANITLPVYTLKERCLQVVRSLVKPENYRRLDI |
| | 213(Y185F)) | VRSLFEDLEDHPNVQKDLERLTQERIAHQRMGD |
| 150 | VHLpep5a | PSLNVDGQPIFANITLPVYTLKERCLQVVRSLVKPEN |
| | (VHL(138-213)) | YRRLDIVRSLYEDLEDHPNVQKDLERLTQERIAHQRM |
| | | GD |
| 151 | VHLpep5b | PSLNVDGQPIFANITLPVYTLKERCLQVVRSLVKPEN |
| | (VHL(138-213 | YRRLDIVRSLFEDLEDHPNVQKDLERLTQERIAHQRM |
| | (Y185F)) | GD |
| 152 | VHLpep6a | MEAGRPRPVLRSVNSREPSQVIFCNRSPRVVLPVWLN |
| | (VHL(54-213)) | FDGEPQPYPTLPPGTGRRIHSYRGHLWLFRDAGTHDG |
| | | LLVNQTELFVPSLNVDGQPIFANITLPVYTLKERCLQ |
| | | VVRSLVKPENYRRLDIVRSLYEDLEDHPNVQKDLERL |
| | | TQERIAHQRMGD |
| 153 | VHLpep6b | MEAGRPRPVLRSVNSREPSQVIFCNRSPRVVLPVWLN |
| | (VHL(54-213 | FDGEPQPYPTLPPGTGRRIHSYRGHLWLFRDAGTHDG |
| | (Y185F)) | LLVNQTELFVPSLNVDGQPIFANITLPVYTLKERCLQ |
| | | VVRSLVKPENYRRLDIVRSLFEDLEDHPNVQKDLERL |
| | | TQERIAHQRMGD |
| 154 | TRIM21 (full) | MASAARLTMMWEEVTCPICLDPFVEPVSIECGHSFCQ |
| | | ECISQVGKGGGSVCPVCRQRFLLKNLRPNRQLANMVN |
| | | NLKEISQEAREGTQGERCAVHGERLHLFCEKDGKALC |
| | | WVCAQSRKHRDHAMVPLEEAAQEYQEKLQVALGELRR |
| | | KQELAEKLEVEIAIKRADWKKTVETQKSRIHAEFVQQ |
| | | KNFLVEEEQRQLQELEKDEREQLRILGEKEAKLAQQS |
| | | QALQELISELDRRCHSSALELLQEVIIVLERSESWNL |
| | | KDLDITSPELRSVCHVPGLKKMLRTCAVHITLDPDTA |
| | | NPWLILSEDRRQVRLGDTQQSIPGNEERFDSYPMVLG |
| | | AQHFHSGKHYWEVDVTGKEAWDLGVCRDSVRRKGHFL |
| | | LSSKSGFWTIWLWNQKYEAGTYPQTPLHLQVPPCQVG |
| | | IFLDYEAGMVSFYNITDHGSLIYSFSECAFTGPLRPF |
| | | FSPGFNDGGKNTAPLTLCPLNIGSQGSTDY |
| 155 | TRIM21pep | MASAARLTMMWEEVTCPICLDPFVEPVSIECGHSFCQ |
| | (TRIM21(1-277)) | ECISQVGKGGGSVCPVCRQRFLLKNLRPNRQLANMVN |
| | | NLKEISQEAREGTQGERCAVHGERLHLFCEKDGKALC |
| | | WVCAQSRKHRDHAMVPLEEAAQEYQEKLQVALGELRR |
| | | KQELAEKLEVEIAIKRADWKKTVETQKSRIHAEFVQQ |
| | | KNFLVEEEQRQLQELEKDEREQLRILGEKEAKLAQQS |
| | | QALQELISELDRRCHSSALELLQEVIIVLERSESWNL |
| | | KDLDITSPELRSVCHVPG |
| 156 | SOCS1 (full) | MVAHNQVAADNAVSTAAEPRRRPEPSSSSSSSPAAPA |
| | | RPRPCPAVPAPAPGDTHFRTFRSHADYRRITRASALL |
| | | DACGFYWGPLSVHGAHERLRAEPVGTFLVRDSRQRNC |
| | | FFALSVKMASGPTSIRVHFQAGRFHLDGSRESFDCLF |
| | | ELLEHYVAAPRRMLGAPLRQRRVRPLQELCRQRIVAT |
| | | VGRENLARIPLNPVLRDYLSSFPFQI |
| 157 | SOCS1pep | VRPLQELCRQRIVATVGRENLARIPLNPVLRDYLSSF |
| | (SOCS1(170-211)) | PFQI |
| 158 | CRB (full) | MAGEGDQQDAAHNMGNHLPLLPAESEEEDEMEVEDQD |
| | | SKEAKKPNIINFDTSLPTSHTYLGADMEEFHGRTLHD |
| | | DDSCQVIPVLPQVMMILIPGQTLPLQLFHPQEVSMVR |
| | | NLIQKDRTFAVLAYSNVQEREAQFGTTAEIYAYREEQ |
| | | DFGIEIVKVKAIGRQRFKVLELRTQSDGIQQAKVQIL |
| | | PECVLPSTMSAVQLESLNKCQIFPSKPVSREDQCSYK |
| | | WWQKYQKRKFHCANLTSWPRWLYSLYDAETLMDRIKK |
| | | QLREWDENLKDDSLPSNPIDFSYRVAACLPIDDVLRI |
| | | QLLKIGSAIQRLRCELDIMNKCTSLCCKQCQETEITT |
| | | KNEIFSLSLCGPMAAYVNPHGYVHETLTVYKACNLNL |
| | | IGRPSTEHSWFPGYAWTVAQCKICASHIGWKFTATKK |
| | | DMSPQKFWGLTRSALLPTIPDTEDEISPDKVILCL |

### E3 Ligase Recruiting Domain

In embodiments, the degradation moiety is an E3 ligase recruiting moiety. An E3 ligase recruiting moiety is a protein, peptide, and/or small molecule that interacts with an endogenous E3 ligase, E3 ligase complex, or E3 ligase accessory protein to recruit the E3 ligase to the target protein.

Proteins and Peptide E3 Ligase Recruiting Domains

In embodiments the E3 ligase recruiting moiety is a protein or a peptide. The protein or peptide may interact with any E3 ligase and/or E3 ligase complex. Table 9 lists some examples of proteins and peptides that may be used as an E ligase recruiting moiety.

In embodiments, E3 ligase recruiting moiety is an Fc domain or portion thereof that interacts with an endogenous ubiquitin ligase. In embodiments, the Fc domain or portion thereof, interacts with TRIM21. In some embodiments, the Fc domain comprises one or more mutations relative to a wild-type Fc domain.

In some embodiments, the E3 ligase recruiting domain is an IgG1 Fc (also called hFc and Fc herein) domain or portion thereof that interacts with an endogenous ubiquitin ligase such as TRIM21. In some embodiments, the IgG1 Fc domain comprises one or more mutations relative to a wild-type IgG1 Fc domain. In some embodiments, the IgG1 Fc domain comprises one or more amino acid substitutions at positions 233, 234, 235, 236, 237, 238, 239, 253, 254, 255, 256, 258, 264, 265, 267, 268, 269, 270, 272, 276, 280, 285, 286, 288, 290, 293, 295, 296, 297, 298, 301, 303, 305, 307, 309, 311, 312, 315, 317, 322, 326, 327, 329, 330, 331, 332, 333, 334, 337, 338, 339, 360, 362, 376, 378, 380, 382, 392, 414, 415, 424, 430, 433, 434, 435, and/or 436 according to the EU numbering system. For example, in some embodiments, the IgG1 Fc domain comprises a mutation at a position selected from 239, 297, and 433 according to the EU numbering system.

In embodiments, the E3 ligase recruiting domain is IgG1 Fc. or an active fragment thereof.

In some embodiments, the E3 ligase recruiting domain is IkBalpha or an IkBalpha peptide that binds to the beta-TrCP subunit of the SCF E3 ligase complex. e.g., as described in U.S. Pat. No. 7,208,157, which is herein incorporated by reference in its entirety. In embodiments, the E3 ligase recruiting domain is IkBalpha or an active fragment thereof. In embodiments, the active fragment of IkBalpha is IkBalpha pep.

In embodiments, the E3 ligase recruiting domain comprises a sequence has is at least 80%, 85%, 90%, 91%, 92%, 93%, 94%, 95%, 96%, 97%, 98%, or 99% percent similarity and/or percent identity to any one of peptides and proteins listed in Table 9. In embodiments, the E3 ligase recruiting domain comprises 80% to 100%, 85% to 100%, 90% to 100%, 95% to 100%, 97% to 100%, 99% to 100%, 90% to 99%, 95% to 99%, or 97% to 99% percent similarity and/or percent identity to any one of peptides and proteins listed in Table 9.

In embodiments, the E3 ligase recruiting domain includes a C-terminal and/or N-terminal cysteine to provide a site for conjugation an EEV and/or targeting moiety as described herein. The addition of a C-terminal and/or N-terminal cysteine may be denoted by a -cys (or -Cys) on the N-terminal and/or C-terminal side of the E3 ligase recruiting domain. In embodiments, E3 recruiting domains having listed herein may include a C-terminal and/or a N-terminal cysteine.

TABLE 9
Protein and Peptide E3 ligase recruiting domains.

| SEQ | E3 ligase | |
|---|---|---|
| ID | recruiting | |
| NO: | domain | Sequence (N to C) |
| 159 | IgG1 Fc | DKTHTCPPCPAPELLGGPSVFLFPPKPKDTLMISRTPEVTCVVV |
| | | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALHNHYTQKS |
| | | LSLSPGK |
| 160 | IgG1 FC | DKTHTCPPCPAPELLGGPSVFLFPPKPKDTLMISRTPEVTCVVV |
| | (N297A) | DVSHEDPEVKFNWYVDGVEVHNAKTKPREEQYASTYRVVSVLTV |
| | | LHQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALHNHYTQKS |
| | | LSLSPGK |
| 161 | IgG1 FC | DKTHTCPPCPAPELLGGPSVFLFPPKPKDTLMISRTPEVTCVVV |
| | (H433A) | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALANHYTQKS |
| | | LSLSPGK |
| 162 | IgG1 Fc | DKTHTCPPCPAPELLGGPCVFLFPPKPKDTLMISRTPEVTCVVV |
| | (S239) | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALHNHYTQKS |
| | | LSLSPGK |
| 163 | IgG1 | DKTHTCPPCPAPELLGGPSVFLFPPKPKDTLMISRTPEVTCVVV |
| | (N297A; | DVSHEDPEVKFNWYVDGVEVHNAKTKPREEQYASTYRVVSVLTV |
| | H433A) | LHQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALANHYTQKS |
| | | LSLSPG |
| 164 | IgG1 | DKTHTCPPCPAPELLGGPCVFLFPPKPKDTLMISRTPEVTCVVV |
| | (N97A; | DVSHEDPEVKFNWYVDGVEVHNAKTKPREEQYASTYRVVSVLTV |
| | S239C) | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQKS |
| | | LSLSPGK |
| 165 | IgG1 | DKTHTCPPCPAPELLGGPCVFLFPPKPKDTLMISRTPEVTCVVV |
| | (H433A; | DVSHEDPEVKFNWYVDGVEVHNAKTKPREEQYNSTYRVVSVLTV |
| | S239C | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALANHYTQKS |
| | | LSLSPGK |
| 166 | IgG1 | DKTHTCPPCPAPELLGGPCVFLFPPKPKDTLMISRTPEVTCVVV |
| | (HN29A; | DVSHEDPEVKFNWYVDGVEVHNAKTKPREEQYASTYRVVSVLTV |
| | H433A; | LHQDWLNGKEYKCKVSNKALPAPIEKTISKAKGQPREPQVYTLP |
| | S239C) | PSRDELTKNQVSLTCLVKGFYPSDIAVEWESNGQPENNYKTTPP |
| | | VLDSDGSFFLYSKLTVDKSRWQQGNVFSCSVMHEALANHYTQKS |
| | | LSLSPGK |
| 167 | IkBalpha | MFQAAERPQEWAMEGPRDGLKKERLLDDRHDSGLDSMKDEEYEQ |
| | | MVKELQEIRLEPQEVPRGSEPWKQQLTEDGDSFLHLAIIHEEKA |
| | | LTMEVIRQVKGDLAFLNFQNNLQQTPLHLAVITNQPEIAEALLG |
| | | AGCDPELRDFRGNTPLHLACEQGCLASVGVLTQSCTTPHLHSIL |
| | | KATNYNGHTCLHLASIHGYLGIVELLVSLGADVNAQEPCNGRTA |
| | | LHLAVDLQNPDLVSLLLKCGADVNRVTYQGYSPYQLTWGRPSTR |
| | | IQQQLGQLTLENLQMLPESEDEESYDTESEFTEFTEDELPYDDC |
| | | VFGGQRLTL |
| 168 | IkBalpha | DRHDSGLDSM |
| | pep | |

### Small Molecule E3 Ligase Recruiting Domains

In embodiments the E3 ligase recruiting moiety is a small molecule or a peptidomimetic.

In embodiments, the E3 ligase recruiting domain is peptidomimetic. In embodiments, the peptidomimetic E3 ligase recruiter domain includes VH032 (Galdeano, et al., J. Med. Chem. (2014), 57, 20:504-513), VH101 (Ishida et al., SLAS Discov. (2021), 26(4): 484-502), VH298 (Frost et al., Nat. Commun. (2016), 6:133312), LCL161 (Troup et al., Exploration of Target Anti-tumor Therapy (2020), 1:273-312. doi.org/10.37349/etat.2020.00018), methylbestin, derivatives thereof, and combinations thereof. In embodiments, VH032, VH101, VH298, derivatives thereof, and combinations thereof interact with the VHL E3 ligase. In embodiments, LCL161, methylbestin, derivatives thereof, and combinations thereof interact with the cIAP E3 ligase.

In embodiments, the E3 ligase recruiting domain is a small molecule. In embodiments, the small molecule is thalidomide, pomalidomide, lenalidomide, bardoxolone methyl, nutlin-3, nimbolide, indisulam, derivatives thereof, and combinations thereof (Ishida et al., SLAS Discov. (2021), 26(4): 484-502; Sun et al., Nature, Signal Transduction and Targeted Therapy (2019), 4(64), doi.org/10.1038/s41392-019-0101-6; Troup et al., Exploration of Target Anti-tumor Therapy (2020), 1:273-312. doi.org/10.37349/etat.2020.00018). In embodiments, thalidomide, lenalidomide, pomalidomide, and derivatives thereof, interact with CRBN. In embodiments, nutlin-3 and derivatives thereof interact with MDM2. In embodiments, nimbolide and derivatives thereof interact with RNF 114.

In embodiments, indisulam and derivatives thereof interact with the CUL4 CLR E3 ligase complex. In embodiments, indisulam and derivatives thereof interact with substrate receptor protein DCAF15.

In some embodiments, the E3 ligase recruiting domain is compound 159 or compound 160. Compound 159, which binds to and recruits VHL. Compound 160 binds to and recruits CRBN.

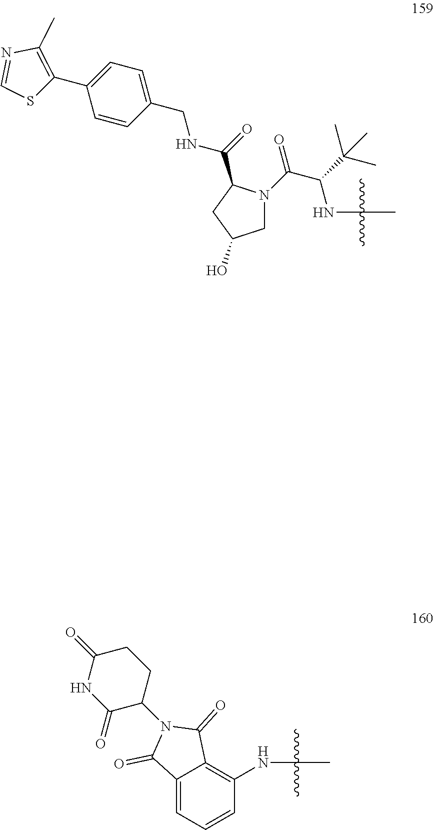 [SMILES: *N[C@H](C(=O)N1C[C@H](O)C[C@H]1C(=O)NCc1ccc(-c2scnc2C)cc1)C(C)(C)C ; *Nc1cccc2c1C(=O)N(C1CCC(=O)NC1=O)C2=O]

### Linkers and Additional Amino Acid Sequences

The components of the degradation construct are operably linked through one or more linkers. As used herein, the term “operably linked” refers to a direct or indirect covalent linking between the components of a degradation construct. Thus, the degradation moiety and the targeting moiety and/or bispecific construct that are operably linked may be directly covalently coupled to one another. Conversely, the degradation moiety and the targeting moiety and/or bispecific construct may be connected by mutual covalent linking to an intervening component (e.g., a flanking sequence or linker). For example, in embodiments where the degradation construct includes a degradation moiety and a bispecific construct, the degradation moiety and the second targeting moiety may be separately directly linked to targeting moiety; or the degradation moiety may be directly linked to the targeting moiety and the second targeting moiety may be directly linked to the degradation moiety.

The term “linker” as used herein refers any bond, small molecule, peptide sequence, or other vehicle that physically links the components of the degradation construct. Linkers can be susceptible to or be substantially resistant to acid-induced cleavage, light-induced cleavage, peptidase-induced cleavage, esterase-induced cleavage, and/or disulfide bond cleavage. Linkers are classified based on the presence of one or more chemical motifs such as, for example, including a disulfide group, a hydrazine group or peptide (cleavable), or a thioester group (non-cleavable). Linkers also include charged linkers, and hydrophilic forms thereof as known in the art.

Suitable linkers for linking the components of the degradation constructs of the present disclosure include a natural linker, an empirical linker, or a combination of natural and/or empirical linkers. Natural linkers are derived from the amino acid linking sequence of multi-domain proteins, which are naturally present between protein domains. Properties of natural linkers such as, for example, length, hydrophobicity, amino acid residues, and/or secondary structure can be exploited to confer desirable properties to a multi-domain compound that includes natural linkers connecting the components of the degradation constructs of the present disclosure.

The studies of linkers in natural multi-domain proteins have led to the generation of many empirical linkers with various sequences and conformations for the construction of recombinant fusion proteins. Empirical linkers are often classified as three types: flexible linkers, rigid linkers, and cleavable linkers. Flexible linkers can provide a certain degree of movement or interaction at the joined components. Flexible linkers typically include small, non-polar (e.g., Gly) or polar (e.g., Ser or Thr) amino acids, which provide flexibility, and allow for mobility of the connected components. Rigid linkers can successfully keep a fixed distance between the degradation moiety and the targeting moiety and/or bispecific construct of the degradation constructs to maintain their independent functions, which can provide efficient separation of targeting moiety and the degradation moiety and/or sufficiently reduce interference between targeting moiety and the degradation moiety.

In some embodiments, the degradation constructs described herein comprise at least one amino acid that is used to connect components of the degradation construct. The amino acid linker may be referred to as a linker peptide. The linker peptide may contain 2, 3, 4, 5, 6, 7, 8, 9, 10, 11, 12, 13, 14, 15, 16, 17, 18, 19, 20 or more amino acids.

In some embodiments, the degradation constructs include described a linker peptide. In some embodiments, the degradation construct includes two, three, four, or five linker peptides. For example, in embodiments where the targeting moiety is a bispecific construct, the first targeting moiety and the second targeting moiety may be connected by a peptide linker and the bispecific construct may be connected to the degradation moiety through a second linker pendetide. In embodiments, the degradation construct does not include a peptide linker. In embodiments, all the components of the degradation construct are directly linked. In embodiments, some components of the depredation construct are directly linked and other are linked via a peptide linker. For example, in embodiments where the targeting moiety is a bispecific construct, the first targeting moiety and the second targeting moiety may be linked via a peptide linker and the bispecific construct is directly linked to the degradation moiety. In embodiments where the targeting moiety is a bispecific construct, the first targeting moiety and the second targeting moiety may be directly linked and the bispecific construct may be linked to the degradation construct via a peptide linker.

In embodiments, the degradation construct includes one or more linkers of Table 10.

TABLE 10
Polypeptide linker sequences

| SEQ | |
|---|---|
| ID | |
| NO: | Sequence |
| 169 | GGGGS |
| 170 | GGGGSGGGGS |
| 171 | GGGGAAA |
| 172 | GGGGAA |
| 173 | GGGGA |
| 174 | GGGGSAAA |
| 175 | GGGGSAA |
| 176 | GGGGSA |
| 177 | GGGGSGGGGSAAA |
| 178 | GGGGSGGGGSAA |
| 179 | GGGGSGGGGSA |
| 180 | SGGGG |
| 181 | SGGGGSGGGG |
| 182 | AAAGGGG |
| 183 | AAGGGG |
| 184 | AGGGG |
| 185 | AAASGGGG |
| 186 | AASGGGG |
| 187 | AGGGGSA |
| 188 | AAASGGGGSGGGG |
| 189 | AASGGGGSGGGG |
| 190 | ASGGGGSGGGG |
| 191 | SSGGGGSS |
| 192 | SGGGGSS |
| 193 | SGGGGS |
| 194 | SSGGGSS |
| 195 | SSGGGS |
| 196 | SGGGSS |
| 197 | SSGGGGSSAA |
| 198 | SSGGGGSAA |
| 199 | SSGGGGAA |
| 200 | AASSGGGGSS |
| 201 | AASGGGGSS |
| 202 | AAGGGGSS |

In some embodiments, the natural linker or empirical linker is covalently attached to the targeting moiety or bispecific construct, degradation moiety, or both, using bioconjugation chemistries. Bioconjugation chemistries are well known in the art and include but are not limited to, NLHS-ester ligation, isocyanate ligation, isothiocyanate ligation, benzoyl fluoride ligation, maleimide conjugation, iodoacetamide conjugation, 2-thiopyridine disulfide exchange, 3-arylpropionitrile conjugation, diazonium salt conjugation, PAD conjugation, and Mannich ligation.

In some embodiments, the natural linker or empirical linker, the targeting moiety or the bispecific construct, the degradation moiety, or both, may include one or more unnatural amino acids that allow for bioorthogonal conjugation reactions. As used herein, “bioorthogonal conjugation” refers to a conjugation reaction that uses one or more unnatural amino acids or modified amino acids as a starting reagent. Examples of bioorthogonal conjugation reactions include but are not limited to, Staudinger ligation, copper-catalyzed azide-alkyne cycloaddition, strain promoted [3+2] cycloadditions, tetrazine ligation, metal-catalyzed coupling reactions, or oxime-hydrazone ligations. Examples of non-natural amino acids include, but are not limited to, azidohomoalanine, 2 homopropargylglycine, 3 homoallylglycine, 4 p-acetyl-Phe, 5 p-azido-Phe, 3-(6-acetylnaphthalen-2-ylamino)-2-aminopropanoic acid, NE-(cyclooct-2-yn-1-yloxy)carbonyl)L-lysine, NE-2-azideoethyloxycarbonyl-L-lysine, Ne-p-azidobenzyloxycarbonyl lysine, Propargyl-L-lysine, or trans-cyclooct-2-ene lysine.

In some embodiments, the linker is derived from a small molecule, such as a polymer. Example polymer linkers include but are not limited to, poly-ethylene glycol, poly(N-isopropylacrylamide), and N,N′-dimethylacrylamide)-co-4-phenylazophenyl acrylate. The small molecule linkers generally include one or more reactive handles allowing conjugation to the degradation moiety, targeting moiety, or both. In some embodiments, the reactive handle allows for a bioconjugation or bioorthogonal conjugation. In some embodiments, the reactive handle allows for any organic reaction compatible with conjugating a linker to the targeting moiety, degradation moiety, or both.

The linker may be conjugated at any amino acid location of the targeting moiety or bispecific moiety, and degradation moiety. For example, the linker may be conjugated to the N-terminus, C-terminus, or any amino acid between.

In embodiments where the degradation construct includes additional domains, the additional domains may be operably coupled to each other and/or the targeting moiety and/or degradation moiety using one or more of the linkers disclosed elsewhere herein.

In some embodiments where the degradation construct includes a targeting moiety or bispecific construct, and a degradation moiety comprised of amino acids that are operably coupled by peptide linkers, the degradation construct may be produced by expression in a host cell. In some embodiments where the degradation construct includes a targeting moiety and a degradation moiety comprised of amino acids that are operably coupled by peptide linkers, the degradation construct may be produced by solid phase peptide synthesis.

In embodiments, the degradation construct and/or one or more components of the degradation construct may include a protein tag. The protein tag may be a purification tag or a cell signaling tag. In embodiments, the targeting moiety or the bispecific construct, degradation moiety, and/or the full degradation construct may include a protein tag.

In some embodiments, the degradation construct and/or one or more components of the degradation construct include a protein tag such as glutathione S-transferase (GST), histidine, a histidine peptide, hemagglutinin, and/or a FLAG tag. Examples of some protein tags are provided in Table 11 below. In some embodiments, the protein tag is on the N-terminus of degradation construct and/or one or more components of the degradation construct sequence. In some embodiments, the protein tag is on the C-terminus of the degradation construct and/or one or more components of the degradation construct. In embodiments, degradation construct and/or one or more components of the degradation construct includes a protein tag of one of the tags listed in Table 11.

TABLE 11
Examples of Some Polypeptide Protein Tags

| SEQ | | |
|---|---|---|
| ID NO: | Name | Sequence (N to C) |
| 203 | GST-FLAG | MSPILGYWKIKGLVQPTRL |
| | | LLEYLEEKYEEHLYERDEG |
| | | DKWRNKKFELGLEFPNLPY |
| | | YIDGDVKLTQSMAIIRYIA |
| | | DKHNMLGGCPKERAEISML |
| | | EGAVLDIRYGVSRIAYSKD |
| | | FETLKVDFLSKLPEMLKMF |
| | | EDRLCHKTYLNGDHVTHPD |
| | | FMLYDALDVVLYMDPMCLD |
| | | AFPKLVCFKKRIEAIPQID |
| | | KYLKSSKYIAWPLQGWQAT |
| | | FGGGDHPPKSDLVPRGSDY |
| | | KDDDDK |
| 204 | GST-FLAG, | SKYIAWPLQGWQATFGGGD |
| | partial | HPPKSDLVPRGSDYKDDDD |
| | sequence | K |
| 205 | GST-His | MSPILGYWKIKGLVQPTRL |
| | | LLEYLEEKYEEHLYERDEG |
| | | DKWRNKKFELGLEFPNLPY |
| | | YIDGDVKLTQSMAIIRYIA |
| | | DKHNMLGGCPKERAEISML |
| | | EGAVLDIRYGVSRIAYSKD |
| | | FETLKVDFLSKLPEMLKMF |
| | | EDRLCHKTYLNGDHVTHPD |
| | | FMLYDALDVVLYMDPMCLD |
| | | AFPKLVCFKKRIEAIPQID |
| | | KYLKSSKYIAWPLQGWQAT |
| | | FGGGDHPPKSDGSTSGSGH |
| | | HHHHHSAGLVPRGS |
| 206 | His-FLAG | HHHHHHDYKDDDDK |
| 207 | Hemagglutinin | YPYDVPDYA |
| | (HA) | |
| 208 | His-pep | MHHHHHHENLYFQG |
| 209 | mammalian | MGWSCIILFLVATATGVHS |
| | signal peptide | |

### Exemplary Degradation Constructs

One or more targeting moieties may be combined with one or more degradation moieties to form a degradation construct. The targeting moieties may be combined with one or more of the degradation moieties in any suitable order, e.g., at the N-terminus or C-terminus of the targeting moiety, or on the side chain of an internal amino acid in the targeting moiety's amino acid sequence. Similarly, the linkage to the degradation moiety may occur at any suitable position, e.g., at the N-terminus or C-terminus of the degradation moiety, or on the side chain of an internal amino acid in the degradation moiety's amino acid sequence. The targeting moiety may be directly linked or linked by a peptide linker or synthetic linker to the degradation moiety described herein and/or one or more amino acids (e.g., 1, 2, 3, 4, 5, 6, 7, 8, 9, 10 or more amino acids).

Matrix 1, Matrix 2, Matrix 3, Matrix 4, Matrix 5, Matrix 6, and Matrix 7 provide example degradation constructs that include a degradation moiety and a targeting moiety. The sequences for the degradation moieties are provided in Tables 8 and 9. The sequences for the targeting moieties are provided in Tables 4A-B.

Matrix 1: Example degradation constructs

| | | Degradation Moiety | | | |
|---|---|---|---|---|---|
| | | VHL | VHLpep1a | VHLpep2a | VHLpep3a |
| Targeting | BC1 | X | X | X | X |
| Moiety | BC2 | X | X | X | X |
| | BC3, BC4, BC5, BC6, BC7, | X | X | X | X |
| | BC8, BC9, BC10, BC11, BC12, | | | | |
| | or BCM | | | | |
| | 7D12 | X | X | X | X |
| | KRAS VHH | X | X | X | X |
| | HRAS VHH | X | X | X | X |
| | ASCVHH | X | X | X | X |
| | ASC VHLPETG | X | X | X | X |
| | K19 | X | X | X | X |
| | K13 | X | X | X | X |
| | MOP4 | X | X | X | X |
| | MOP3+ | X | X | X | X |
| | MOP4 | X | X | X | X |
| | Bispecific constructs | | | | |
| | BC1 with 7D12 | X | X | X | X |
| | BC2 with 7D12 | X | X | X | X |
| | KRAS VHH with 7D12 | X | X | X | X |
| | HRAS VHH with 7D12 | X | X | X | X |
| | ASCVHH with 7D12 | X | X | X | X |
| | ASC VH LPETG with 7D12 | X | X | X | X |
| | K19 with 7D12 | X | X | X | X |
| | K13 with 7D12 | X | X | X | X |
| | MOP4 with 7D12 | X | X | X | X |
| | MOP3+ with 7D12 | X | X | X | X |
| | MOP4 with 7D12 | X | X | X | X |
| | BC3, BC4, BC5, BC6, BC7, | X | X | X | X |
| | BC8, BC9, BC10, BC11, BC12, | | | | |
| | or BCM with 7D12 | | | | |

Matrix 2: Example degradation constructs

| | | Degradation Moiety | | | |
|---|---|---|---|---|---|
| | | VHLpep3a | VHLpep3b | VHLpep4a | VHLpep4b |
| Targeting | BC1 | X | X | X | X |
| Moiet | BC2 | X | X | X | X |
| | BC3, BC4, BC5, BC6, BC7, | X | X | X | X |
| | BC8, BC9, BC10, BC11, BC12, or BC14 | | | | |
| | KRAS VHH | X | X | X | X |
| | HRAS VHH | X | X | X | X |
| | ASCVHH | X | X | X | X |
| | ASC VH LPETG | X | X | X | X |
| | K19 | X | X | X | X |
| | K13 | X | X | X | X |
| | MOP4 | X | X | X | X |
| | MOP3+ | X | X | X | X |
| | M0P4 | X | X | X | X |
| | Bispecific constructs | | | | |
| | BC1 with 7D12 | X | X | X | X |
| | BC2 with 7D12 | X | X | X | X |
| | KRAS VHH with 7D12 | X | X | X | X |
| | HRAS VHH with 7D12 | X | X | X | X |
| | ASCVHH with 7D12 | X | X | X | X |
| | ASC VH LPETG with 7D12 | X | X | X | X |
| | K19 with7D12 | X | X | X | X |
| | K13 with7D12 | X | X | X | X |
| | MOP4 with 7D12 | X | X | X | X |
| | MOP3+ with 7D12 | X | X | X | X |
| | MOP4 with 7D12 | X | X | X | X |
| | BC3, BC4, BC5, BC6, BC7, BC8, | X | X | X | X |
| | BC9, BC10, BC11, BC12, or BCM with 7D12 | | | | |

Matrix 3: Example degradation constructs

| | | Degradation Moiety | | | |
|---|---|---|---|---|---|
| | | SOCS1 | SOCS1pep | Compound 59 | Compound 60 |
| Targeting | BC1 | X | X | X | X |
| Moiety | BC2 | X | X | X | X |
| | BC3, BC4, BC5, BC6, BC7, | X | X | X | X |
| | BC8, BC9, BC10, BC11, | | | | |
| | BC12, or BCM | | | | |
| | KRAS VHH | X | X | X | X |
| | HRAS VHH | X | X | X | X |
| | ASCVHH | X | X | X | X |
| | ASC VH LPETG | X | X | X | X |
| | K19 | X | X | X | X |
| | K13 | X | X | X | X |
| | MOP4 | X | X | X | X |
| | MOP3+ | X | X | X | X |
| | MOP4 | X | X | X | X |
| | Bispecific constructs | | | | |
| | BC1 with 7D12 | X | X | X | X |
| | BC2 with 7D12 | X | X | X | X |
| | KRAS VHH with 7D12 | X | X | X | X |
| | HRAS VHH with 7D12 | X | X | X | X |
| | ASCVHH with 7D12 | X | X | X | X |
| | ASC VH LPETG with 7D12 | X | X | X | X |
| | K19 with 7D12 | X | X | X | X |
| | K13 with 7D12 | X | X | X | X |
| | MOP4 with 7D12 | X | X | X | X |
| | MOP3+ with 7D12 | X | X | X | X |
| | MOP4 with 7D12 | X | X | X | X |
| | BC3, BC4, BC5, BC6, BC7, | X | X | X | X |
| | BC8, BC9, BC10, BC11, | | | | |
| | BC12, or BCM with 7D12 | | | | |

Matrix 4: Example degradation constructs

| | | Degradation Moiety | | | | |
|---|---|---|---|---|---|---|
| | | | | IgG1 | IgG1 Fc | |
| | | | IgG1 Fc | Fc | (N297A); | IgG1 Fc |
| | | IgG1 Fc | (N297A) | (H433) | H433) | (S239C) |
| Targeting | BC1 | X | X | X | X | X |
| Moriety | BC2 | X | X | X | X | X |
| | BC3, BC4, BC5, BC6, BC7, | X | X | X | X | X |
| | BC8, BC9, BC10, BC11, | | | | | |
| | BC12, or BCM | | | | | |
| | KRAS VHH | X | X | X | X | X |
| | HRAS VHH | X | X | X | X | X |
| | ASCVHH | X | X | X | X | X |
| | ASC VH LPETG | X | X | X | X | X |
| | K19 | X | X | X | X | X |
| | K13 | X | X | X | X | X |
| | MOP4 | X | X | X | X | X |
| | MOP3+ | X | X | X | X | X |
| | MOP4 | X | X | X | X | X |
| | Bispecific constructs | | | | | |
| | BC1 with 7D12 | X | X | X | X | X |
| | BC2 with 7D12 | X | X | X | X | X |
| | KRAS VHH with 7D12 | X | X | X | X | X |
| | HRAS VHH with 7D12 | X | X | X | X | X |
| | ASCVHH with 7D12 | X | X | X | X | X |
| | ASC VH LPETG with 7D12 | X | X | X | X | X |
| | K19 with 7D12 | X | X | X | X | X |
| | K13 with 7D12 | X | X | X | X | X |
| | MOP4 with 7D12 | X | X | X | X | X |
| | MOP3+ with 7D12 | X | X | X | X | X |
| | MOP4 with 7D12 | X | X | X | X | X |
| | BC3, BC4, BC5, BC6, BC7, | X | X | X | X | X |
| | BC8, BC9, BC10, BC11, | | | | | |
| | BC12, or BCM with 7D12 | | | | | |

Matrix 5: Example degradation constructs

| | | Degradation Moiety | | | | |
|---|---|---|---|---|---|---|
| | | | | | | NKBalpha |
| | | TRIM21 | TRIM21pep | CRB | NKBalpha | pep |
| Targeting | BC1 | X | X | X | X | X |
| Moriety | BC2 | X | X | X | X | X |
| | BC3, BC4, BC5, BC6, BC7, | X | X | X | X | X |
| | BC8, BC9, BC10, BC11, | | | | | |
| | BC12, or BCM | | | | | |
| | KRAS VHH | X | X | X | X | X |
| | HRAS VHH | X | X | X | X | X |
| | ASCVHH | X | X | X | X | X |
| | ASC VHLPETG | X | X | X | X | X |
| | K19 | X | X | X | X | X |
| | K13 | X | X | X | X | X |
| | MOP4 | X | X | X | X | X |
| | MOP3+ | X | X | X | X | X |
| | M0P4 | X | X | X | X | X |
| | Bispecific constructs | | | | | |
| | BC1 with 7D12 | X | X | X | X | X |
| | BC2 with7D12 | X | X | X | X | X |
| | KRAS VHH with 7D12 | X | X | X | X | X |
| | HRAS VHH with 7D12 | X | X | X | X | X |
| | ASCVHH with 7D12 | X | X | X | X | X |
| | ASC VH LPETG with 7D12 | X | X | X | X | X |
| | K19 with7D12 | X | X | X | X | X |
| | K13 with7D12 | X | X | X | X | X |
| | MOP4 with 7D12 | X | X | X | X | X |
| | MOP3+ with 7D12 | X | X | X | X | X |
| | MOP4 with 7D12 | X | X | X | X | X |
| | BC3, BC4, BC5, BC6, BC7, | X | X | X | X | X |
| | BC8,BC9, BC10, BC11, | | | | | |
| | BC12, or BCM with 7D12 | | | | | |

Matrix 6: Example degradation constructs

| | | Degradation Moiety | | |
|---|---|---|---|---|
| | | | | IgGl Fc |
| | | IgG1 Fc | IgG1 Fc | (S239C; |
| | | (S239C; | (S239C; | H433A; |
| | | N297A) | H433A) | N297A) |
| Targeting | BC1 | X | X | X |
| Moiety | BC2 | X | X | X |
| | BC3, BC4, BC5, BC6, BC7, BC8, | X | X | X |
| | BC9, BC10, BC11, BC12, or | | | |
| | BCM | | | |
| | 7D12 | X | X | X |
| | KRAS VHH | X | X | X |
| | HRAS VHH | X | X | X |
| | ASCVHH | X | X | X |
| | ASC VH LPETG | X | X | X |
| | K19 | X | X | X |
| | K13 | X | X | X |
| | MOP4 | X | X | X |
| | MOP3+ | X | X | X |
| | MOP4 | X | X | X |
| | Bispecific constructs | | | |
| | BC1 with 7D12 | X | X | X |
| | BC2 with 7D12 | X | X | X |
| | KRAS VHH with 7D12 | X | X | X |
| | HRAS VHH with 7D12 | X | X | X |
| | ASCVHH with 7D12 | X | X | X |
| | ASC VH LPETG with 7D12 | X | X | X |
| | K19 with 7D12 | X | X | X |
| | K13 with 7D12 | X | X | X |
| | MOP4 with 7D12 | X | X | X |
| | MOP3+ with 7D12 | X | X | X |
| | MOP4 with 7D12 | X | X | X |
| | BC3, BC4, BC5, BC6, BC7, BC8, | X | X | X |
| | BC9, BC10, BC11, BC12, or | | | |
| | BCM with 7D12 | | | |

Matrix 7: Example degradation constructs

| | | Degradation Moeity | | | |
|---|---|---|---|---|---|
| | | VHLpep5a | VHLpep5b | VHLpep6a | VHLpep6b |
| Targeting | BC1 | X | X | X | X |
| Moiety | BC2 | X | X | X | X |
| | BC3, BC4, BC5, BC6, BC7, | X | X | X | X |
| | BC8, BC9, BC10, BC11, | | | | |
| | BC12, or BCM | | | | |
| | KRAS VHH | X | X | X | X |
| | HRAS VHH | X | X | X | X |
| | ASCVHH | X | X | X | X |
| | ASC VHLPETG | X | X | X | X |
| | K19 | X | X | X | X |
| | K13 | X | X | X | X |
| | MOP4 | X | X | X | X |
| | MOP3+ | X | X | X | X |
| | MOP4 | X | X | X | X |
| | Bispecific constructs | | | | |
| | BC1 with 7D12 | X | X | X | X |
| | BC2 with 7D12 | X | X | X | X |
| | KRAS VHH with 7D12 | X | X | X | X |
| | HRAS VHH with 7D12 | X | X | X | X |
| | ASCVHH with 7D12 | X | X | X | X |
| | ASC VH LPETG with 7D12 | X | X | X | X |
| | K19 with 7D12 | X | X | X | X |
| | K13 with 7D12 | X | X | X | X |
| | MOP4 with 7D12 | X | X | X | X |
| | MOP3+ with 7D12 | X | X | X | X |
| | MOP4 with 7D12 | X | X | X | X |
| | BC3, BC4, BC5, BC6, BC7, | X | X | X | X |
| | BC8, BC9, BC10, BC11, | | | | |
| | BC12, or BCM with 7D12 | | | | |

Similar degradation constructs targeting IRF-5 may also be prepared as indicted in Matrix 1-7 above.

The amino acid sequence of examples of some degradation constructs is provided in Table 12. Construct details are written N to C. Although no linker is not explicitly included in the construct details, a linker may be present a person of ordinary skill in the art would be able to determine the linker via methods known in the art. Additionally, N terminal and or C terminal protein tags may be present and a person or ordinary skill in the art would be able to determine the protein tag sequence via methods known in the art.

TABLE 12
Examples of Some Degradation Constructs

| SEQ | | |
|---|---|---|
| ID | Construct | |
| NO: | specifics | Sequence |
| 210 | Cys-K19- | CDLGKKLLEAARAGQDDEVRILMANGADVNASDRWGWTPLHLAAWWG |
| | TRIM21₁₋₂₇₇ | HLEIVEVLLKRGADVSAADLHGQSPLHLAAMVGHLEIVEVLLKYGAD |
| | | VNAKDTMGATPLHLAARSGHLEIVEELLKNGADMNAQDKFGKTTFDI |
| | | STDNGNEDLAEILQKLGGGGSASAARLTMMWEEVTCPICLDPFVEPV |
| | | SIECGHSFCQECISQVGKGGGSVCPVCRQRFLLKNLRPNRQLANMVN |
| | | NLKEISQEAREGTQGERCAVHGERLHLFCEKDGKALCWVCAQSRKHR |
| | | DHAMVPLEEAAQEYQEKLQVALGELRRKQELAEKLEVEIAIKRADWK |
| | | KTVETQKSRIHAEFVQQKNFLVEEEQRQLQELEKDEREQLRILGEKE |
| | | AKLAQQSQALQELISELDRRCHSSALELLQEVIIVLERSESWNLKDL |
| | | DITSPELRSVCHVPG |
| 211 | TRIM21₁₋₂₇₇ | MASAARLTMMWEEVTCPICLDPFVEPVSIECGHSFCQECISQVGKGG |
| | K19-Cys | GSVCPVCRQRFLLKNLRPNRQLANMVNNLKEISQEAREGTQGERCAV |
| | | HGERLHLFCEKDGKALCWVCAQSRKHRDHAMVPLEEAAQEYQEKLQV |
| | | ALGELRRKQELAEKLEVEIAIKRADWKKTVETQKSRIHAEFVQQKNF |
| | | LVEEEQRQLQELEKDEREQLRILGEKEAKLAQQSQALQELISELDRR |
| | | CHSSALELLQEVIIVLERSESWNLKDLDITSPELRSVCHVPGGGGGS |
| | | DLGKKLLEAARAGQDDEVRILMANGADVNASDRWGWTPLHLAAWWGH |
| | | LEIVEVLLKRGADVSAADLHGQSPLHLAAMVGHLEIVEVLLKYGADV |
| | | NAKDTMGATPLHLAARSGHLEIVEELLKNGADMNAQDKFGKTTFDIS |
| | | TDNGNEDLAEILQKLC |
| 212 | Cys-BC2- | CQVQLVESGGGLVQPGGSLTLSCTASGFTLDHYDIGWFRQAPGKERE |
| | VHLpep2a | GVSCINNSDDDTYYADSVKGRFTIFMNNAKDTVYLQMNSLKPEDTAI |
| | | YYCAEARGCKRGRYEYDFWGQGTQVTVSSGGGGSLPVYTLKERCLQV |
| | | VRSLVKPENYRRLDIVRSLYEDLEDHPNV |
| 213 | VHLpep2a- | LPVYTLKERCLQVVRSLVKPENYRRLDIVRSLYEDLEDHPNVGGGGS |
| | BC2-Cys | QVQLVESGGGLVQPGGSLTLSCTASGFTLDHYDIGWFRQAPGKEREG |
| | | VSCINNSDDDTYYADSVKGRFTIFMNNAKDTVYLQMNSLKPEDTAIY |
| | | YCAEARGCKRGRYEYDFWGQGTQVTVSSC |
| 214 | Cys-7D12- | CQVKLEESGGGSVQTGGSLRLTCAASGRTSRSYGMGWFRQAPGKERE |
| | VHLpep2a | FVSGISWRGDSTGYADSVKGRFTISRDNAKNTVDLQMNSLKPEDTAI |
| | | YYCAAAAGSAWYGTLYEYDYWGQGTQVTVSSGGGGSLPVYTLKERCL |
| | | QVVRSLVKPENYRRLDIVRSLYEDLEDHPNV |
| 215 | VHLpep2a- | LPVYTLKERCLQVVRSLVKPENYRRLDIVRSLYEDLEDHPNVGGGGS |
| | 7D12-Cys | QVKLEESGGGSVQTGGSLRLTCAASGRTSRSYGMGWFRQAPGKEREF |
| | | VSGISWRGDSTGYADSVKGRFTISRDNAKNTVDLQMNSLKPEDTAIY |
| | | YCAAAAGSAWYGTLYEYDYWGQGTQVTVSSC |
| 216 | Cys- | CQLQLVESGGGLVQPGGSLKLSCAASGFTFSRYAMSWYRQAPGKERE |
| | ASC^{VHH}_ | SVARISSGGGTIYYADSVKGRFTISREDAKNTVYLQMNSLKPEDTAV |
| | VHLpep2a | YYCYVGGFWGQGTOVTVSSGGGGSLPVYTLKERCLQVVRSLVKPENY |
| | | RRLDIVRSLYEDLEDHPNV |
| 217 | VHLpep2a- | LPVYTLKERCLQVVRSLVKPENYRRLDIVRSLYEDLEDHPNVGGGGS |
| | ASC^{VHH} | QLQLVESGGGLVQPGGSLKLSCAASGFTFSRYAMSWYRQAPGKERES |
| | | VARISSGGGTIYYADSVKGRFTISREDAKNTVYLQMNSLKPEDTAVY |
| | | YCYVGGFWGQGTQVTVSSC |
| 218 | Cys- | CQVQLQESGGGLVQPGGSLKLSCAASGFTFSRYAMSWYRQAPGKERE |
| | ASC^{VHH}_ | SVARISSGGGTIYYADSVKGRFTISREDAKNTVYLQMNSLKPEDTAV |
| | VHL₁₁₀₋₂₁₃ | YYCYVGGFWGQGTQVTVSSGGGGSHSYRGHLWLFRDAGTHDGLLVAQ |
| | | TELFVPSLNVDGQPIFAAITLPVYTLKERCLQVVRSLVKPENYRRLD |
| | | IVRSLYEDLEDHPNVQKDLERLTQERIAHQRMGD |
| 219 | Cys- | CQVQLQESGGGLVQPGGSLKLSCAASGFTFSRYAMSWYRQAPGKERE |
| | ASC^{VHH}_ | SVARISSGGGTIYYADSVKGRFTISREDAKNTVYLQMNSLKPEDTAV |
| | VHL₁₃₈₋₂₁₃ | YYCYVGGFWGQGTQVTVSSGGGGSPSLNVDGQPIFAAITLPVYTLKE |
| | | RCLQVVRSLVKPENYRRLDIVRSLYEDLEDHPNVQKDLERLTQERIA |
| | | HQRMGD |
| 220 | Cys- | CQVQLQESGGGLVQPGGSLKLSCAASGFTFSRYAMSWYRQAPGKERE |
| | ASC^{VHH}_ | SVARISSGGGTIYYADSVKGRFTISREDAKNTVYLQMNSLKPEDTAV |
| | VHL₁₅₂₋₂₁₃ | YYCYVGGFWGQGTOVTVSSGGGGSTLPVYTLKERCLQVVRSLVKPEN |
| | | YRRLDIVRSLYEDLEDHPNVQKDLERLTQERIAHQRMGD |
| 221 | VHL₁₅₂₋₂₁₃- | TLPVYTLKERCLQVVRSLVKPENYRRLDIVRSLYEDLEDHPNVQKDL |
| | ASC^{VHH}_ | ERLTQERIAHQRMGDGGGGSQLQLVESGGGLVQPGGSLKLSCAASGF |
| | Cys | TFSRYAMSWYRQAPGKERESVARISSGGGTIYYADSVKGRFTISRED |
| | | AKNTVYLQMNSLKPEDTAVYYCYVGGFWGQGTQVTVSSC |
| 222 | VHL- | DYKDDDDKGSSGHHHHHHENLYFQGMPRRAENWDEAEVGAEEAGVEE |
| | ASC^{VHH} | YGPEEDGGEESGAEESGPEESGPEELGAEEEMEAGRPRPVLRSVNSR |
| | | EPSQVIFCNRSPRVVLPVWLNFDGEPQPYPTLPPGTGRRIHSYRGHL |
| | | WLFRDAGTHDGLLVNQTELFVPSLNVDGQPIFANITLPVYTLKERCL |
| | | QWRSLVKPENYRRLDIVRSLYEDLEDHPNVQKDLERLTQERIAHQR |
| | | MGDGGGGGEFAMAQVQLQESGGGLVQPGGSLKLSCAASGFTFSRYAM |
| | | SWYRQAPGKERESVARISSGGGTIYYADSVKGRFTISREDAKNTVYL |
| | | QMNSLKPEDTAVYYCYVGGFWGQGTQVTVSSGGQLQLVESGGGLVQP |
| | | GGSLKLSCAASGFTFSRYAMSWYRQAPGKERESVARISSGGGTIYYA |
| | | DSVKGRFTISREDAKNTVYLQMNSLKPEDTAVYYCYVGGFWGQGTQV |
| | | TVSS |
| 223 | VHL₅₄₋₂₁₃- | DYKDDDDKGSSGHHHHHHENLYFQGMEAGRPRPVLRSVNSREPSQVI |
| | ASC^{VHH} | FCNRSPRVVLPVWLNFDGEPQPYPTLPPGTGRRIHSYRGHLWLFRDA |
| | | GTHDGLLVNQTELFVPSLNVDGQPIFANITLPVYTLKERCLQVVRSL |
| | | VKPENYRRLDIVRSLYEDLEDHPNVQKDLERLTQERIAHQRMGDGGG |
| | | GGEFAMAQVQLQESGGGLVQPGGSLKLSCAASGFTFSRYAMSWYRQA |
| | | PGKERESVARISSGGGTIYYADSVKGRFTISREDAKNTVYLQMNSLK |
| | | PEDTAVYYCYVGGFWGQGTQVTVSSGGQLQLVESGGGLVQPGGSLKL |
| | | SCAASGFTFSRYAMSWYRQAPGKERESVARISSGGGTIYYADSVKGR |
| | | FTISREDAKNTVYLQMNSLKPEDTAVYYCYVGGFWGQGTQVTVSS |
| 224 | Cys- | QLQLVESGGGLVQPGGSLKLSCAASGFTFSRYAMSWYRQAPGKERES |
| | ASC^{VHH} | VARISSGGGTIYYADSVKGRFTISREDAKNTVYLQMNSLKPEDTAVY |
| | TRIM21₁₋₂₇₇ | YCYVGGFWGQGTQVTVSSGGGGSASAARLTMMWEEVTCPICLDPFVE |
| | | PVSIECGHSFCQECISQVGKGGGSVCPVCRQRFLLKNLRPNRQLANM |
| | | VNNLKEISQEAREGTQGERCAVHGERLHLFCEKDGKALCWVCAQSRK |
| | | HRDHAMVPLEEAAQEYQEKLQVALGELRRKQELAEKLEVEIAIKRAD |
| | | WKKTVETQKSRIHAEFVQQKNFLVEEEQRQLQELEKDEREQLRILGE |
| | | KEAKLAQQSQALQELISELDRRCHSSALELLQEVIIVLERSESWNLK |
| | | DLDITSPELRSVCHVPGC |
| 225 | TRIM21₁₋₂₇₇ | MASAARLTMMWEEVTCPICLDPFVEPVSIECGHSFCQECISQVGKGG |
| | ASC^{VHH}_ | GSVCPVCRQRFLLKNLRPNRQLANMVNNLKEISQEAREGTQGERCAV |
| | Cys | HGERLHLFCEKDGKALCWVCAQSRKHRDHAMVPLEEAAQEYQEKLQV |
| | | ALGELRRKQELAEKLEVEIAIKRADWKKTVETQKSRIHAEFVQQKNF |
| | | LVEEEQRQLQELEKDEREQLRILGEKEAKLAQQSQALQELISELDRR |
| | | CHSSALELLQEVIIVLERSESWNLKDLDITSPELRSVCHVPGGGGGS |
| | | QLQLVESGGGLVQPGGSLKLSCAASGFTFSRYAMSWYRQAPGKERES |
| | | VARISSGGGTIYYADSVKGRFTISREDAKNTVYLQMNSLKPEDTAVY |
| | | YCYVGGFWGQGTQVTVSSC |
| 226 | VHL152-213- | TLPVYTLKERCLQVVRSLVKPENYRRLDIVRSLYEDLEDHPNVQKDL |
| | ASC^{VHH}_ | ERLTQERIAHQRMGDGGGGSQLQLVESGGGLVQPGGSLKLSCAASGF |
| | TRIM21₁₋₂₇₇ | TFSRYAMSWYRQAPGKERESVARISSGGGTIYYADSVKGRFTISRED |
| | -Cys | AKNTVYLQMNSLKPEDTAVYYCYVGGFWGQGTQVTVSSGGGGSASAA |
| | | RLTMMWEEVTCPICLDPFVEPVSIECGHSFCQECISQVGKGGGSVCP |
| | | VCRQRFLLKNLRPNRQLANMVNNLKEISQEAREGTQGERCAVHGERL |
| | | HLFCEKDGKALCWVCAQSRKHRDHAMVPLEEAAQEYQEKLQVALGEL |
| | | RRKQELAEKLEVEIAIKRADWKKTVETQKSRIHAEFVQQKNFLVEEE |
| | | QRQLQELEKDEREQLRILGEKEAKLAQQSQALQELISELDRRCHSSA |
| | | LELLQEVIIVLERSESWNLKDLDITSPELRSVCHVPGC |
| 227 | Cys- | CTLPVYTLKERCLQWRSLVKPENYRRLDIVRSLYEDLEDHPNVQKD |
| | VHL₁₅₂₋₂₁₃- | LERLTQERIAHQRMGDGGGGSQLQLVESGGGLVQPGGSLKLSCAASG |
| | ASC^{VHH}_ | FTFSRYAMSWYRQAPGKERESVARISSGGGTIYYADSVKGRFTISRE |
| | linker- | DAKNTVYLQMNSLKPEDTAVYYCYVGGFWGQGTQVTVSSGGGGSVRP |
| | SOCS1pep | LQELCRQRIVATVGRENLARIPLNPVLRDYLSSFPFQI |
| 228 | SOCS1pep- | VRPLQELCRQRIVATVGRENLARIPLNPVLRDYLSSFPFQIGGGGST |
| | VHL₁₅₂₋₂₁₃- | LPVYTLKERCLQVVRSLVKPENYRRLDIVRSLYEDLEDHPNVQKDLE |
| | ASC^{VHH}_ | RLTQERIAHQRMGDGGGGSQLQLVESGGGLVQPGGSLKLSCAASGFT |
| | Cys | FSRYAMSWYRQAPGKERESVARISSGGGTIYYADSVKGRFTISREDA |
| | | KNTVYLQMNSLKPEDTAVYYCYVGGFWGQGTQVTVSSC |
| 229 | Cys- | CTLPVYTLKERCLQVVRSLVKPENYRRLDIVRSLYEDLEDHPNVQKD |
| | VHL₁₅₂₋₂₁₃- | LERLTQERIAHQRMGDGGGGSGGGGSQVQLVESGGGLVQPGGSLTLS |
| | -BC2- | CTASGFTLDHYDIGWFRQAPGKEREGVSCINNSDDDTYYADSVKGRF |
| | SOCS1pep | TIFMNNAKDTVYLQMNSLKPEDTAIYYCAEARGCKRGRYEYDFWGQG |
| | | TQVTVSSGGGGSGGGGSVRPLQELCRQRIVATVGRENLARIPLNPVL |
| | | RDYLSSEPFQI |
| 230 | VHL₁₅₂₋₂₁₃ | TLPVYTLKERCLQVVRSLVKPENYRRLDIVRSLFEDLEDHPNVQKDL |
| | _{(Y185F)}-BC2- | ERLTQERIAHQRMGDGGGGSGGGGSQVQLVESGGGLVQPGGSLTLSC |
| | Cys | TASGFTLDHYDIGWFRQAPGKEREGVSCINNSDDDTYYADSVKGRFT |
| | | IFMNNAKDTVYLQMNSLKPEDTAIYYCAEARGCKRGRYEYDFWGQGT |
| | | QVTVSSC |
| 231 | Cys-BC2- | CQVQLVESGGGLVQPGGSLTLSCTASGFTLDHYDIGWFRQAPGKERE |
| | SOCS1pep | GVSCINNSDDDTYYADSVKGRFTIFMNNAKDTVYLQMNSLKPEDTAI |
| | | YYCAEARGCKRGRYEYDFWGQGTQVTVSSGGGGSVRPLQELCRQRIV |
| | | ATVGRENLARIPLNPVLRDYLSSFPFQI |
| 232 | SOCS1pep- | VRPLQELCRQRIVATVGRENLARIPLNPVLRDYLSSFPFQIGGGGSQ |
| | BC2-Cys | VQLVESGGGLVQPGGSLTLSCTASGFTLDHYDIGWFRQAPGKEREGV |
| | | SCINNSDDDTYYADSVKGRFTIFMNNAKDTVYLQMNSLKPEDTAIYY |
| | | CAEARGCKRGRYEYDFWGQGTQVTVSSC |
| 233 | Cys-BC2- | CQVQLVESGGGLVQPGGSLTLSCTASGFTLDHYDIGWFRQAPGKERE |
| | TRIM21₁₋₂₇₇ | GVSCINNSDDDTYYADSVKGRFTIFMNNAKDTVYLQMNSLKPEDTAI |
| | | YYCAEARGCKRGRYEYDFWGQGTQVTVSSGGGGSGGGGSASAARLTM |
| | | MWEEVTCPICLDPFVEPVSIECGHSFCQECISQVGKGGGSVCPVCRQ |
| | | RFLLKNLRPNRQLANMVNNLKEISQEAREGTQGERCAVHGERLHLFC |
| | | EKDGKALCWVCAQSRKHRDHAMVPLEEAAQEYQEKLQVALGELRRKQ |
| | | ELAEKLEVEIAIKRADWKKTVETQKSRIHAEFVQQKNFLVEEEQRQL |
| | | QELEKDEREQLRILGEKEAKLAQQSQALQELISELDRRCHSSALELL |
| | | QEVIIVLERSESWNLKDLDITSPELRSVCHVPG |
| 234 | TRIM21₁₋₂₇₇ | HHHHHHGDDDDKASAARLTMMWEEVTCPICLDPFVEPVSIECGHSFC |
| | -BC2- | QECISQVGKGGGSVCPVCRQRFLLKNLRPNRQLANMVNNLKEISQEA |
| | Cys | REGTQGERCAVHGERLHLFCEKDGKALCWVCAQSRKHRDHAMVPLEE |
| | | AAQEYQEKLQVALGELRRKQELAEKLEVEIAIKRADWKKTVETQKSR |
| | | IHAEFVQQKNFLVEEEQRQLQELEKDEREQLRILGEKEAKLAQQSQA |
| | | LQELISELDRRCHSSALELLQEVIIVLERSESWNLKDLDITSPELRS |
| | | VCHVPGGGGGSGGGGSQVQLVESGGGLVQPGGSLTLSCTASGFTLDH |
| | | YDIGWFRQAPGKEREGVSCINNSDDDTYYADSVKGRFTIFMNNAKDT |
| | | VYLQMNSLKPEDTAIYYCAEARGCKRGRYEYDFWGQGTQVTVSSC |
| 235 | VHL152-213- | TLPVYTLKERCLQVVRSLVKPENYRRLDIVRSLYEDLEDHPNVQKDL |
| | BC2- | ERLTQERIAHQRMGDGGGGSGGGGSQVQLVESGGGLVQPGGSLTLSC |
| | TRIM21₁₋₂₇₇ | TASGFTLDHYDIGWFRQAPGKEREGVSCINNSDDDTYYADSVKGRFT |
| | -Cys | IFMNNAKDTVYLQMNSLKPEDTAIYYCAEARGCKRGRYEYDFWGQGT |
| | | QVTVSSGGGGSGGGGSASAARLTMMWEEVTCPICLDPFVEPVSIECG |
| | | HSFCQECISQVGKGGGSVCPVCRQRFLLKNLRPNRQLANMVNNLKE1 |
| | | SQEAREGTQGERCAVHGERLHLFCEKDGKALCWVCAQSRKHRDHAMV |
| | | PLEEAAQEYQEKLQVALGELRRKQELAEKLEVEIAIKRADWKKTVET |
| | | QKSRIHAEFVQQKNFLVEEEQRQLQELEKDEREQLRILGEKEAKLAQ |
| | | QSQALQELISELDRRCHSSALELLQEVIIVLERSESWNLKDLDITSP |
| | | ELRSVCHVPGC |
| 236 | Cys-7D12- | CQVKLEESGGGSVQTGGSLRLTCAASGRTSRSYGMGWFRQAPGKERE |
| | SOCS1pep | FVSGISWRGDSTGYADSVKGRFTISRDNAKNTVDLQMNSLKPEDTAI |
| | | YYCAAAAGSAWYGTLYEYDYWGQGTQVTVSSGGGGSVRPLQELCRQR |
| | | IVATVGRENLARIPLNPVLRDYLSSFPFQI |
| 237 | SOCS1pep- | VRPLQELCRQRIVATVGRENLARIPLNPVLRDYLSSFPFQIGGGGSQ |
| | 7D12-Cys | VKLEESGGGSVQTGGSLRLTCAASGRTSRSYGMGWFRQAPGKEREFV |
| | | SGISWRGDSTGYADSVKGRFTISRDNAKNTVDLQMNSLKPEDTAIYY |
| | | CAAAAGSAWYGTLYEYDYWGQGTQVTVSSC |
| 238 | Cys- | CQLQLVESGGGLVQPGGSLKLSCAASGFTFSRYAMSWYRQAPGKERE |
| | ASC^{VHH}_ | SVARISSGGGTIYYADSVKGRFTISREDAKNTVYLQMNSLKPEDTAV |
| | SOCS1pep | YYCYVGGFWGQGTQVTVSSGGGGSVRPLQELCRQRIVATVGRENLAR |
| | | IPLNPVLRDYLSSFPFQI |
| 239 | SOCS1pep- | VRPLQELCRQRIVATVGRENLARIPLNPVLRDYLSSFPFQIGGGGSQ |
| | ASC^{VHH}_ | LQLVESGGGLVQPGGSLKLSCAASGFTFSRYAMSWYRQAPGKERESV |
| | Cys | ARISSGGGTIYYADSVKGRFTISREDAKNTVYLQMNSLKPEDTAVYY |
| | | CYVGGFWGQGTQVTVSSC |
| 240 | Cys-7D12- | CQVKLEESGGGSVQTGGSLRLTCAASGRTSRSYGMGWFRQAPGKERE |
| | K19- | FVSGISWRGDSTGYADSVKGRFTISRDNAKNTVDLQMNSLKPEDTAI |
| | VHLpep2a | YYCAAAAGSAWYGTLYEYDYWGQGTQVTVSSGGGGSGGGGSDLGKKL |
| | | LEAARAGQDDEVRILMANGADVNASDRWGWTPLHLAAWWGHLEIVEV |
| | | LLKRGADVSAADLHGQSPLHLAAMVGHLEIVEVLLKYGADVNAKDTM |
| | | GATPLHLAARSGHLEIVEELLKNGADMNAQDKFGKTTFDISTDNGNE |
| | | DLAEILQKLGGGGSLPVYTLKERCLQVVRSLVKPENYRRLDIVRSLY |
| | | EDLEDHPNV |
| 241 | Cys-7D12- | CQVKLEESGGGSVQTGGSLRLTCAASGRTSRSYGMGWFRQAPGKERE |
| | K19- | FVSGISWRGDSTGYADSVKGRFTISRDNAKNTVDLQMNSLKPEDTAI |
| | VHL₁₅₂₋₂₁₃ | YYCAAAAGSAWYGTLYEYDYWGQGTQVTVSSGGGGSGGGGSDLGKKL |
| | | LEAARAGQDDEVRILMANGADVNASDRWGWTPLHLAAWWGHLEIVEV |
| | | LLKRGADVSAADLHGQSPLHLAAMVGHLEIVEVLLKYGADVNAKDTM |
| | | GATPLHLAARSGHLEIVEELLKNGADMNAQDKFGKTTFDISTDNGNE |
| | | DLAEILQKLGGGGSTLPVYTLKERCLQVVRSLVKPENYRRLDIVRSL |
| | | YEDLEDHPNVQKDLERLTQERIAHQRMGD |
| 242 | Cys-7D12- | CQVKLEESGGGSVQTGGSLRLTCAASGRTSRSYGMGWFRQAPGKERE |
| | K19- | FVSGISWRGDSTGYADSVKGRFTISRDNAKNTVDLQMNSLKPEDTAI |
| | TRIM21₁₋₂₇₇ | YYCAAAAGSAWYGTLYEYDYWGQGTQVTVSSGGGGSGGGGSDLGKKL |
| | | LEAARAGQDDEVRILMANGADVNASDRWGWTPLHLAAWWGHLEIVEV |
| | | LLKRGADVSAADLHGQSPLHLAAMVGHLEIVEVLLKYGADVNAKDTM |
| | | GATPLHLAARSGHLEIVEELLKNGADMNAQDKFGKTTFDISTDNGNE |
| | | DLAEILQKLGGGGSASAARLTMMWEEVTCPICLDPFVEPVSIECGHS |
| | | FCQECISQVGKGGGSVCPVCRQRFLLKNLRPNRQLANMVNNLKEISQ |
| | | EAREGTQGERCAVHGERLHLFCEKDGKALCWVCAQSRKHRDHAMVPL |
| | | EEAAQEYQEKLQVALGELRRKQELAEKLEVEIAIKRADWKKTVETQK |
| | | SRIHAEFVQQKNFLVEEEQRQLQELEKDEREQLRILGEKEAKLAQQS |
| | | QALQELISELDRRCHSSALELLQEVIIVLERSESWNLKDLDITSPEL |
| | | RSVCHVPG |
| 243 | Cys-K19- | CDLGKKLLEAARAGQDDEVRILMANGADVNASDRWGWTPLHLAAWWG |
| | 7D12- | HLEIVEVLLKRGADVSAADLHGQSPLHLAAMVGHLEIVEVLLKYGAD |
| | VHL₁₅₂₋₂₁₃ | VNAKDTMGATPLHLAARSGHLEIVEELLKNGADMNAQDKFGKTTFDI |
| | | STDNGNEDLAEILQKLGGGGSGGGGSQVKLEESGGGSVQTGGSLRLT |
| | | CAASGRTSRSYGMGWFRQAPGKEREFVSGISWRGDSTGYADSVKGRF |
| | | TISRDNAKNTVDLQMNSLKPEDTAIYYCAAAAGSAWYGTLYEYDYWG |
| | | QGTQVTVSSGGGGSTLPVYTLKERCLQVVRSLVKPENYRRLDIVRSL |
| | | YEDLEDHPNVQKDLERLTQERIAHQRMGD |
| 244 | Cys-K19- | CDLGKKLLEAARAGQDDEVRILMANGADVNASDRWGWTPLHLAAWWG |
| | 7D12- | HLEIVEVLLKRGADVSAADLHGQSPLHLAAMVGHLEIVEVLLKYGAD |
| | VHLpep | VNAKDTMGATPLHLAARSGHLEIVEELLKNGADMNAQDKFGKTTFDI |
| | | STDNGNEDLAEILQKLGGGGSGGGGSQVKLEESGGGSVQTGGSLRLT |
| | | CAASGRTSRSYGMGWFRQAPGKEREFVSGISWRGDSTGYADSVKGRF |
| | | TISRDNAKNTVDLQMNSLKPEDTAIYYCAAAAGSAWYGTLYEYDYWG |
| | | QGTQVTVSSGGGGSLPVYTLKERCLQVVRSLVKPENYRRLDIVRSLY |
| | | EDLEDHPNV |
| 245 | Cys-K19 | CDLGKKLLEAARAGQDDEVRILMANGADVNASDRWGWTPLHLAAWWG |
| | 7D12- | HLEIVEVLLKRGADVSAADLHGQSPLHLAAMVGHLEIVEVLLKYGAD |
| | SOCS1pep | VNAKDTMGATPLHLAARSGHLEIVEELLKNGADMNAQDKFGKTTFDI |
| | | STDNGNEDLAEILQKLGGGGSGGGGSQVKLEESGGGSVQTGGSLRLT |
| | | CAASGRTSRSYGMGWFRQAPGKEREFVSGISWRGDSTGYADSVKGRF |
| | | TISRDNAKNTVDLQMNSLKPEDTAIYYCAAAAGSAWYGTLYEYDYWG |
| | | QGTQVTVSSGGGGSVRPLQELCRQRIVATVGRENLARIPLNPVLRDY |
| | | LSSFPFQI |
| 246 | ASC^{VHH}_ | QLQLVESGGGLVQPGGSLKLSCAASGFTFSRYAMSWYRQAPGKERES |
| | hFc(N297A) | VARISSGGGTIYYADSVKGRFTISREDAKNTVYLQMNSLKPEDTAVY |
| | | YCYVGGFWGQGTQVTVSSGGGGSDKTHTCPPCPAPELLGGPSVFLFP |
| | | PKPKDTLMISRTPEVTCVVVDVSHEDPEVKFNWYVDGVEVHNAKTKP |
| | | REEQYASTYRVVSVLTVLHQDWLNGKEYKCKVSNKALPAPIEKTISK |
| | | AKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEAL |
| | | HNHYTQKSLSLSPGC |
| 247 | ASCVHH | QLQLVESGGGLVQPGGSLKLSCAASGFTFSRYAMSWYRQAPGKERES |
| | hFc(WT) | VARISSGGGTIYYADSVKGRFTISREDAKNTVYLQMNSLKPEDTAVY |
| | | YCYVGGFWGQGTQVTVSSGGGGSDKTHTCPPCPAPELLGGPSVFLFP |
| | | PKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISK |
| | | AKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEAL |
| | | HNHYTQKSLSLSPGK |
| 248 | ASCVHH | QLQLVESGGGLVQPGGSLKLSCAASGFTFSRYAMSWYRQAPGKERES |
| | hFc | VARISSGGGTIYYADSVKGRFTISREDAKNTVYLQMNSLKPEDTAVY |
| | (N297A/H4 | YCYVGGFWGQGTQVTVSSGGGGSDKTHTCPPCPAPELLGGPSVFLFP |
| | 33A) | PKPKDTLMISRTPEVTCVVVDVSHEDPEVKFNWYVDGVEVHNAKTKP |
| | | REEQYASTYRVVSVLTVLHQDWLNGKEYKCKVSNKALPAPIEKTISK |
| | | AKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEAL |
| | | ANHYTQKSLSLSPGC |
| 249 | ASC^{VHH}_ | QLQLVESGGGLVQPGGSLKLSCAASGFTFSRYAMSWYRQAPGKERES |
| | hFc(N297A/ | VARISSGGGTIYYADSVKGRFTISREDAKNTVYLQMNSLKPEDTAVY |
| | S239C) | YCYVGGFWGQGTQVTVSSGGGGSDKTHTCPPCPAPELLGGPCVFLFP |
| | | PKPKDTLMISRTPEVTCVVVDVSHEDPEVKFNWYVDGVEVHNAKTKP |
| | | REEQYASTYRVVSVLTVLHQDWLNGKEYKCKVSNKALPAPIEKTISK |
| | | AKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEAL |
| | | HNHYTQKSLSLSPGK |
| 250 | ASC^{VHH}_ | QLQLVESGGGLVQPGGSLKLSCAASGFTFSRYAMSWYRQAPGKERES |
| | Mb-Cys | VARISSGGGTIYYADSVKGRFTISREDAKNTVYLQMNSLKPEDTAVY |
| | | YCYVGGFWGQGTQVTVSSEPKSSDKTHTCPPCPGQPREPQVYTLPPS |
| | | REEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGC |
| 251 | Cys- | CQLQLVESGGGLVQPGGSLKLSCAASGFTFSRYAMSWYRQAPGKERE |
| | ASC^{VHH}_ | SVARISSGGGTIYYADSVKGRFTISREDAKNTVYLQMNSLKPEDTAV |
| | Mb-VHL₁₅₂₋ | YYCYVGGFWGQGTQVTVSSEPKSSDKTHTCPPCPGQPREPQVYTLPP |
| | ₂₁₃ | SREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGG |
| | | GGSTLPVYTLKERCLQVVRSLVKPENYRRLDIVRSLYEDLEDHPNVQ |
| | | KDLERLTQERIAHQRMGD |
| 252 | VHL₁₅₂₋₂₁₃- | TLPVYTLKERCLQVVRSLVKPENYRRLDIVRSLYEDLEDHPNVQKDL |
| | ASCVHH | ERLTQERIAHQRMGDGGGGSQLQLVESGGGLVQPGGSLKLSCAASGF |
| | Mb-Cys | TFSRYAMSWYRQAPGKERESVARISSGGGTIYYADSVKGRFTISRED |
| | | AKNTVYLQMNSLKPEDTAVYYCYVGGFWGQGTQVTVSSEPKSSDKTH |
| | | TCPPCPGQPREPQVYTLPPSREEMTKNQVSLTCLVKGFYPSDIAVEW |
| | | ESNGQPENNYKTTPPVLDSDGSFFLYSKLTVDKSRWQQGNVFSCSVM |
| | | HEALHNHYTQKSLSLSPGC |
| 253 | BC2-hFc | QVQLVESGGGLVQPGGSLTLSCTASGFTLDHYDIGWFRQAPGKEREG |
| | (N297A) | VSCINNSDDDTYYADSVKGRFTIFMNNAKDTVYLQMNSLKPEDTAIY |
| | | YCAEARGCKRGRYEYDFWGQGTQVTVSSGGGGSDKTHTCPPCPAPEL |
| | | LGGPSVFLFPPKPKDTLMISRTPEVTCVVVDVSHEDPEVKFNWYVDG |
| | | VEVHNAKTKPREEQYASTYRVVSVLTVLHQDWLNGKEYKCKVSNKAL |
| | | PAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNV |
| | | FSCSVMHEALHNHYTQKSLSLSPGC |
| 254 | BC2- | QVQLVESGGGLVQPGGSLTLSCTASGFTLDHYDIGWFRQAPGKEREG |
| | hFc | VSCINNSDDDTYYADSVKGRFTIFMNNAKDTVYLQMNSLKPEDTAIY |
| | (S239C) | YCAEARGCKRGRYEYDFWGQGTQVTVSSGGGGSDKTHTCPPCPAPEL |
| | | LGGPCVFLFPPKPKDTLMISRTPEVTCVVVDVSHEDPEVKFNWYVDG |
| | | VEVHNAKTKPREEQYASTYRVVSVLTVLHQDWLNGKEYKCKVSNKAL |
| | | PAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNV |
| | | FSCSVMHEALHNHYTQKSLSLSPG |
| 255 | BC2-hFc- | QVQLVESGGGLVQPGGSLTLSCTASGFTLDHYDIGWFRQAPGKEREG |
| | Cys | VSCINNSDDDTYYADSVKGRFTIFMNNAKDTVYLQMNSLKPEDTAIY |
| | (S239C) | YCAEARGCKRGRYEYDFWGQGTQVTVSSGGGGSDKTHTCPPCPAPEL |
| | | LGGPCVFLFPPKPKDTLMISRTPEVTCVVVDVSHEDPEVKFNWYVDG |
| | | VEVHNAKTKPREEQYASTYRVVSVLTVLHQDWLNGKEYKCKVSNKAL |
| | | PAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNV |
| | | FSCSVMHEALHNHYTQKSLSLSPGC |
| 256 | BC2-hFc | QVQLVESGGGLVQPGGSLTLSCTASGFTLDHYDIGWFRQAPGKEREG |
| | (N297A/H4 | VSCINNSDDDTYYADSVKGRFTIFMNNAKDTVYLQMNSLKPEDTAIY |
| | 33A)-Cys | YCAEARGCKRGRYEYDFWGQGTQVTVSSGGGGSDKTHTCPPCPAPEL |
| | | LGGPSVFLFPPKPKDTLMISRTPEVTCVVVDVSHEDPEVKFNWYVDG |
| | | VEVHNAKTKPREEQYASTYRVVSVLTVLHQDWLNGKEYKCKVSNKAL |
| | | PAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNV |
| | | FSCSVMHEALANHYTQKSLSLSPGC |
| 257 | BC2-Mb- | QVQLVESGGGLVQPGGSLTLSCTASGFTLDHYDIGWFRQAPGKEREG |
| | Cys | VSCINNSDDDTYYADSVKGRFTIFMNNAKDTVYLQMNSLKPEDTAIY |
| | | YCAEARGCKRGRYEYDFWGQGTQVTVSSEPKSSDKTHTCPPCPGQPR |
| | | EPQVYTLPPSREEMTKNQVSLTCLVKGFYPSDIAVEWESNGQPENNY |
| | | kttppvldsdgsfflyskltvdksrwqqgnvfscsvmhealhnhytq |
| | | KSLSLSPGC |
| 258 | Cys-BC2- | CQVQLVESGGGLVQPGGSLTLSCTASGFTLDHYDIGWFRQAPGKERE |
| | Mb-VHL₁₅₂₋₂₁₃ | GVSCINNSDDDTYYADSVKGRFTIFMNNAKDTVYLQMNSLKPEDTAI |
| | | YYCAEARGCKRGRYEYDFWGQGTQVTVSSEPKSSDKTHTCPPCPGQP |
| | | REPQVYTLPPSREEMTKNQVSLTCLVKGFYPSDIAVEWESNGQPENN |
| | | ykttppvldsdgsfflyskltvdksrwqqgnvfscsvmhealhnhyt |
| | | QKSLSLSPGGGGGSGGGGSTLPVYTLKERCLQVVRSLVKPENYRRLD |
| | | IVRSLYEDLEDHPNVQKDLERLTQERIAHQRMGD |
| 259 | VHL₁₅₂₋₂₁₃₋ | TLPVYTLKERCLQVVRSLVKPENYRRLDIVRSLYEDLEDHPNVQKDL |
| | BC2-Mb- | ERLTQERIAHQRMGDGGGGSGGGGSQVQLVESGGGLVQPGGSLTLSC |
| | Cys | TASGFTLDHYDIGWFRQAPGKEREGVSCINNSDDDTYYADSVKGRFT |
| | | IFMNNAKDTVYLQMNSLKPEDTAIYYCAEARGCKRGRYEYDFWGQGT |
| | | QVTVSSEPKSSDKTHTCPPCPGQPREPQVYTLPPSREEMTKNQVSLT |
| | | CLVKGFYPSDIAVEWESNGQPENNYKTTPPVLDSDGSFFLYSKLTVD |
| | | KSRWQQGNVFSCSVMHEALHNHYTQKSLSLSPGC |
| 260 | KRasVHH | EVQLVESGGGSVQTGGSLRLSCAVSGNIGSSYCMGWFRQAPGKKREA |
| | linker- | VARIVRDGATGYADYVKGRFTISRDSAKNTLYLQMNRLIPEDTAIYY |
| | hFc(N297A) | CAADLPPGCLTQAIWNFGYRGQGTLVTVSSGGGGSDKTHTCPPCPAP |
| | | ELLGGPSVFLFPPKPKDTLMISRTPEVTCVVVDVSHEDPEVKFNWYV |
| | | DGVEVHNAKTKPREEQYASTYRVVSVLTVLHQDWLNGKEYKCKVSNK |
| | | ALPAPIEKTISKAKGQPREPQVYTLPPSREEMTKNQVSLTCLVKGFY |
| | | PSDIAVEWESNGQPENNYKTTPPVLDSDGSFFLYSKLTVDKSRWQQG |
| | | NVFSCSVMHEALHNHYTQKSLSLSPGC |
| 261 | KRasVHH | EVQLVESGGGSVQTGGSLRLSCAVSGNIGSSYCMGWFRQAPGKKREA |
| | hFc(N297A/ | VARIVRDGATGYADYVKGRFTISRDSAKNTLYLQMNRLIPEDTAIYY |
| | H433A) | CAADLPPGCLTQAIWNFGYRGQGTLVTVSSGGGGSDKTHTCPPCPAP |
| | | ELLGGPSVFLFPPKPKDTLMISRTPEVTCVVVDVSHEDPEVKFNWYV |
| | | DGVEVHNAKTKPREEQYASTYRVVSVLTVLHQDWLNGKEYKCKVSNK |
| | | ALPAPIEKTISKAKGQPREPQVYTLPPSREEMTKNQVSLTCLVKGFY |
| | | PSDIAVEWESNGQPENNYKTTPPVLDSDGSFFLYSKLTVDKSRWQQG |
| | | NVFSCSVMHEALANHYTQKSLSLSPGC |
| 262 | K19-hFc- | DLGKKLLEAARAGQDDEVRILMANGADVNASDRWGWTPLHLAAWWGH |
| | Cys | LEIVEVLLKRGADVSAADLHGQSPLHLAAMVGHLEIVEVLLKYGADV |
| | | NAKDTMGATPLHLAARSGHLEIVEELLKNGADMNAQDKFGKTTFDIS |
| | | TDNGNEDLAEILQKLGGGGSDKTHTCPPCPAPELLGGPSVFLFPPKP |
| | | KDTLMISRTPEVTCVVVDVSHEDPEVKFNWYVDGVEVHNAKTKPREE |
| | | QYASTYRVVSVLTVLHQDWLNGKEYKCKVSNKALPAPIEKTISKAKG |
| | | QPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNH |
| | | YTQKSLSLSPGC |
| 263 | K19- | DLGKKLLEAARAGQDDEVRILMANGADVNASDRWGWTPLHLAAWWGH |
| | hFc(S239C) | LEIVEVLLKRGADVSAADLHGQSPLHLAAMVGHLEIVEVLLKYGADV |
| | | NAKDTMGATPLHLAARSGHLEIVEELLKNGADMNAQDKFGKTTFDIS |
| | | TDNGNEDLAEILQKLGGGGSDKTHTCPPCPAPELLGGPCVFLFPPKP |
| | | KDTLMISRTPEVTCVVVDVSHEDPEVKFNWYVDGVEVHNAKTKPREE |
| | | QYASTYRVVSVLTVLHQDWLNGKEYKCKVSNKALPAPIEKTISKAKG |
| | | QPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNH |
| | | YTQKSLSLSPGK |
| 264 | hFc(S239C) | DKTHTCPPCPAPELLGGPCVFLFPPKPKDTLMISRTPEVTCVVVDVS |
| | -K19 | HEDPEVKFNWYVDGVEVHNAKTKPREEQYASTYRVVSVLTVLHQDWL |
| | | NGKEYKCKVSNKALPAPIEKTISKAKGQPREPQVYTLPPSRDELTKN |
| | | QVSLTCLVKGFYPSDIAVEWESNGQPENNYKTTPPVLDSDGSFFLYS |
| | | KLTVDKSRWQQGNVFSCSVMHEALHNHYTQKSLSLSPGGGGGSGGGG |
| | | SDLGKKLLEAARAGQDDEVRILMANGADVNASDRWGWTPLHLAAWWG |
| | | HLEIVEVLLKRGADVSAADLHGQSPLHLAAMVGHLEIVEVLLKYGAD |
| | | VNAKDTMGATPLHLAARSGHLEIVEELLKNGADMNAQDKFGKTTFDI |
| | | STDNGNEDLAEILQKL |
| 262 | K19-hFc- | DLGKKLLEAARAGQDDEVRILMANGADVNASDRWGWTPLHLAAWWGH |
| | Cys | LEIVEVLLKRGADVSAADLHGQSPLHLAAMVGHLEIVEVLLKYGADV |
| | | NAKDTMGATPLHLAARSGHLEIVEELLKNGADMNAQDKFGKTTFDIS |
| | | TDNGNEDLAEILQKLGGGGSDKTHTCPPCPAPELLGGPSVFLFPPKP |
| | | KDTLMISRTPEVTCVVVDVSHEDPEVKFNWYVDGVEVHNAKTKPREE |
| | | QYASTYRVVSVLTVLHQDWLNGKEYKCKVSNKALPAPIEKTISKAKG |
| | | QPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNH |
| | | YTQKSLSLSPGC |
| 265 | K19-hFc- | DLGKKLLEAARAGQDDEVRILMANGADVNASDRWGWTPLHLAAWWGH |
| | Cys | LEIVEVLLKRGADVSAADLHGQSPLHLAAMVGHLEIVEVLLKYGADV |
| | | NAKDTMGATPLHLAARSGHLEIVEELLKNGADMNAQDKFGKTTFDIS |
| | | TDNGNEDLAEILQKLGGGGSDKTHTCPPCPAPELLGGPSVFLFPPKP |
| | | KDTLMISRTPEVTCVVVDVSHEDPEVKFNWYVDGVEVHNAKTKPREE |
| | | QYASTYRVVSVLTVLHQDWLNGKEYKCKVSNKALPAPIEKTISKAKG |
| | | QPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALANH |
| | | YTQKSLSLSPGC |
| 266 | K19-hFc | DLGKKLLEAARAGQDDEVRILMANGADVNASDRWGWTPLHLAAWWGH |
| | | LEIVEVLLKRGADVSAADLHGQSPLHLAAMVGHLEIVEVLLKYGADV |
| | | NAKDTMGATPLHLAARSGHLEIVEELLKNGADMNAQDKFGKTTFDIS |
| | | TDNGNEDLAEILQKLGGGGSGGGGSEPKSSDKTHTCPPCPAPELLGG |
| | | PCVFLFPPKPKDTLMISRTPEVTCVVVDVSHEDPEVKFNWYVDGVEV |
| | | HNAKTKPREEQYASTYRVVSVLTVLHQDWLNGKEYKCKVSNKALPAP |
| | | IEKTISKAKGQPREPQVYTLPPSRDELTKNQVSLTCLVKGFYPSDIA |
| | | VEWESNGQPENNYKTTPPVLDSDGSFFLYSKLTVDKSRWQQGNVFSC |
| | | SVMHEALHNHYTQKSLSLSPGK |
| 264 | hFc-K19 | DKTHTCPPCPAPELLGGPCVFLFPPKPKDTLMISRTPEVTCVVVDVS |
| | | HEDPEVKFNWYVDGVEVHNAKTKPREEQYASTYRVVSVLTVLHQDWL |
| | | NGKEYKCKVSNKALPAPIEKTISKAKGQPREPQVYTLPPSRDELTKN |
| | | QVSLTCLVKGFYPSDIAVEWESNGQPENNYKTTPPVLDSDGSFFLYS |
| | | KLTVDKSRWQQGNVFSCSVMHEALHNHYTQKSLSLSPGGGGGSGGGG |
| | | SDLGKKLLEAARAGQDDEVRILMANGADVNASDRWGWTPLHLAAWWG |
| | | HLEIVEVLLKRGADVSAADLHGQSPLHLAAMVGHLEIVEVLLKYGAD |
| | | VNAKDTMGATPLHLAARSGHLEIVEELLKNGADMNAQDKFGKTTFDI |
| | | STDNGNEDLAEILQKL |
| 267 | Cys-K19- | CDLGKKLLEAARAGQDDEVRILMANGADVNASDRWGWTPLHLAAWWG |
| | Mb- | HLEIVEVLLKRGADVSAADLHGQSPLHLAAMVGHLEIVEVLLKYGAD |
| | VHL₁₅₂₋₂₁₃ | VNAKDTMGATPLHLAARSGHLEIVEELLKNGADMNAQDKFGKTTFDI |
| | | STDNGNEDLAEILQKLGGGGSEPKSSDKTHTCPPCPGQPREPQVYTL |
| | | PPSREEMTKNQVSLTCLVKGFYPSDIAVEWESNGQPENNYKTTPPVL |
| | | DSDGSFFLYSKLTVDKSRWQQGNVFSCSVMHEALHNHYTQKSLSLSP |
| | | GGGGGSTLPVYTLKERCLQVVRSLVKPENYRRLDIVRSLYEDLEDHP |
| | | NVQKDLERLTQERIAHQRMGD |
| 268 | VHL₁₅₂₋₂₁₃ | TLPVYTLKERCLQVVRSLVKPENYRRLDIVRSLYEDLEDHPNVQKDL |
| | -K19-Mb- | ERLTQERIAHQRMGDGGGGSDLGKKLLEAARAGQDDEVRILMANGAD |
| | Cys | VNASDRWGWTPLHLAAWWGHLEIVEVLLKRGADVSAADLHGQSPLHL |
| | | AAMVGHLEIVEVLLKYGADVNAKDTMGATPLHLAARSGHLEIVEELL |
| | | KNGADMNAQDKFGKTTFDISTDNGNEDLAEILQKLGGGGSEPKSSDK |
| | | THTCPPCPGQPREPQVYTLPPSREEMTKNQVSLTCLVKGFYPSDIAV |
| | | EWESNGQPENNYKTTPPVLDSDGSFFLYSKLTVDKSRWQQGNVFSCS |
| | | VMHEALHNHYTQKSLSLSPGC |
| 269 | KRas^{VHH}_ | EVQLVESGGGSVQTGGSLRLSCAVSGNIGSSYCMGWFRQAPGKKREA |
| | Mb-Cys | VARIVRDGATGYADYVKGRFTISRDSAKNTLYLQMNRLIPEDTAIYY |
| | | CAADLPPGCLTQAIWNFGYRGQGTLVTVSSEPKSSDKTHTCPPCPGQ |
| | | PREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHY |
| | | TQKSLSLSPGC |
| 270 | HRas^{VHH}_ | EVQLLESGGGLVQPGGSLRLSCAASGFTFSTFSMNWVRQAPGKGLEW |
| | Mb-Cys | VSYISRTSKTIYYADSVKGRFTISRDNSKNTLYLQMNSLRAEDTAVY |
| | | YCARGRFFDYWGQGTLVTVSSEPKSSDKTHTCPPCPGQPREPQVYTL |
| | | PPSREEMTKNQVSLTCLVKGFYPSDIAVEWESNGQPENNYKTTPPVL |
| | | DSDGSFFLYSKLTVDKSRWQQGNVFSCSVMHEALHNHYTQKSLSLSP |
| | | GC |
| 271 | K19-Mb- | DLGKKLLEAARAGQDDEVRILMANGADVNASDRWGWTPLHLAAWWGH |
| | Cys | LEIVEVLLKRGADVSAADLHGQSPLHLAAMVGHLEIVEVLLKYGADV |
| | | NAKDTMGATPLHLAARSGHLEIVEELLKNGADMNAQDKFGKTTFDIS |
| | | TDNGNEDLAEILQKLGGGGSEPKSSDKTHTCPPCPGQPREPQVYTLP |
| | | PSREEMTKNQVSLTCLVKGFYPSDIAVEWESNGQPENNYKTTPPVLD |
| | | SDGSFFLYSKLTVDKSRWQQGNVFSCSVMHEALHNHYTQKSLSLSPG |
| | | C |
| 272 | 7D12-Mb- | QVKLEESGGGSVQTGGSLRLTCAASGRTSRSYGMGWFRQAPGKEREF |
| | Cys | VSGISWRGDSTGYADSVKGRFTISRDNAKNTVDLQMNSLKPEDTAIY |
| | | YCAAAAGSAWYGTLYEYDYWGQGTQVTVSSEPKSSDKTHTCPPCPGQ |
| | | PREPQVYTLPPSREEMTKNQVSLTCLVKGFYPSDIAVEWESNGQPEN |
| | | nykttppvldsdgsfflyskltvdksrwqqgnvfscsvmhealhnhy |
| | | TQKSLSLSPGC |
| 273 | VHH^{CARD}_ | QLQLVESGGGLVQPGGSLKLSCAASGFTFSRYAMSWYRQAPGKERES |
| | Mb | VARISSGGGTIYYADSVKGRFTISREDAKNTVYLQMNSLKPEDTAVY |
| | | YCYVGGFWGQGTQVTVSSGGGGSEPKSSDKTHTCPPCPGQPREPQVY |
| | | TLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSL |
| | | SPGK |
| 274 | VHL₅₄₋₂₁₃- | MDYKDDDDKGSSGHHHHHHENLYFQGMEAGRPRPVLRSVNSREPSQV |
| | VHH^{CARD}_ | IFCNRSPRVVLPVWLNFDGEPQPYPTLPPGTGRRIHSYRGHLWLFRD |
| | | AGTHDGLLVNQTELFVPSLNVDGQPIFANITLPVYTLKERCLQVVRS |
| | | LVKPENYRRLDIVRSLYEDLEDHPNVQKDLERLTQERIAHQRMGDGG |
| | | GGGEFAMAQVQLQESGGGLVQPGGSLKLSCAASGFTFSRYAMSWYRQ |
| | | APGKERESVARISSGGGTIYYADSVKGRFTISREDAKNTVYLQMNSL |
| | | KPEDTAVYYCYVGGFWGQGTQVTVSSGGQLQLVESGGGLVQPGGSLK |
| | | LSCAASGFTFSRYAMSWYRQAPGKERESVARISSGGGTIYYADSVKG |
| | | RFTISREDAKNTVYLQMNSLKPEDTAVYYCYVGGFWGQGTQVTVSS |
| 275 | VHL₁₋₁₂₃- | MPRRAENWDEAEVGAEEAGVEEYGPEEDGGEESGAEESGPEESGPEE |
| | K19 | LGAEEEMEAGRPRPVLRSVNSREPSQVIFCNRSPRVVLPVWLNFDGE |
| | | PQPYPTLPPGTGRRIHSYRGHLWLFRDAGTHDGLLVNQTELFVPSLN |
| | | VDGQPIFANITLPVYTLKERCLQVVRSLVKPENYRRLDIVRSLYEDL |
| | | EDHPNVQKDLERLTQERIAHQRMGDGGGGSAAADLGKKLLEAARAGQ |
| | | DDEVRILMANGADVNASDRWGWTPLHLAAWWGHLEIVEVLLKRGADV |
| | | SAADLHGQSPLHLAAMVGHLEIVEVLLKYGADVNAKDTMGATPLHLA |
| | | ARSGHLEIVEELLKNGADMNAQDKFGKTTFDISTDNGNEDLAEILQK |
| | | LGGGGSDYKDDDDK |
| 276 | VHL₁₅₇₋₁₉₄- | MTLKERCLQVVRSLVKPENYRRLDIVRSLYEDLEDHPNVGGGGSAAA |
| | K19 | DLGKKLLEAARAGQDDEVRILMANGADVNASDRWGWTPLHLAAWWGH |
| | | LEIVEVLLKRGADVSAADLHGQSPLHLAAMVGHLEIVEVLLKYGADV |
| | | NAKDTMGATPLHLAARSGHLEIVEELLKNGADMNAQDKFGKTTFDIS |
| | | TDNGNEDLAEILQKLGGGGSDYKDDDDK |

In some embodiments, the degradation construct has at least 80%, 85%, 90%, 91%, 92%, 93%, 94%, 95%, 96%, 97%, 98%, or 99% percent similarity and/or percent identity to any one of constructs listed in Table 12. In embodiments, the degradation construct has 80% to 100%, 85% to 100%, 90% to 100%, 95% to 100%, 97% to 100%, 99% to 100%, 90% to 99%, 95% to 99%, or 97% to 99% percent similarity and/or percent identity to any one of constructs listed in Table 12.

In embodiments, the degradation construct includes a C-terminal and/or N-terminal cysteine to provide a site for conjugation to EEV and/or degradation moiety as described herein. The addition of a C-terminal and/or N-terminal cysteine may be denoted by a -cys (or -Cys) on the N-terminal and/or C-terminal side of the degradation construct. In embodiments, a degradation construct listed in Table 12 may further include a C-terminal and/or a N-terminal cysteine.

### Amino Acid Modifications

The amino acid sequences described herein may be modified.

Examples of conservative amino acid substitutions include substitution of one amino acid for another amino acid within one from one of the following groups: basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). In some embodiments, structurally similar amino acids are substituted to reverse the charge of a residue (e.g., glutamine for glutamic acid or vice-versa, aspartic acid for asparagine or vice-versa). In some embodiments, tyrosine is substituted for phenylalanine or vice-versa. Other non-limiting examples of amino acid substitutions are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In some embodiments, a pharmaceutically acceptable water-soluble polymer may be conjugated to the construct. Non-limiting examples of pharmaceutically acceptable water soluble polymers include polyethylene glycol (PEG), dextran or poly(n-vinyl pyrrolidone)polyethylene glycol, propropylene glycol homopolymers, prolypropylene oxide/ethylene oxide co-polymers, polyoxyethylated polyols, polyvinyl alcohol, polyoxyethylated polyols, polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C1-C10) alkoxy- or aryloxy-polyethylene glycol, carboxymethylcellulose, polyacetals, polyvinyl alcohol (PVA), polyvinyl pyrrolidone, poly-1,3-dioxolane, poly-1,3,6-trioxane, ethylene/maleic anhydride copolymer, poly(β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or and mixtures thereof. In particular embodiments, the TP is a PEGylated. As used herein “PEGylation” refers to the coupling of TP to one or more polyethylene glycol (PEG) residues. In some embodiments, the molecular weight of the PEG is from about 0.1 kDa to about 100 kDa, e.g., about 0.1 kDa, about 1 kDa, about 10 kDa, about 20 kDa, about 30 kDa, about 40 kDa, about 50 kDa, about 60 kDa, about 70 kDa, about 80 kDa, about 90 kDa, and about 100 kDa. In particular embodiments, the PEG is about 1, 2, 3, 4, 5, 6, 7, 8, 9, 10, 15, 20, 25, 30, 35, 40, 45, or 50 kDa, including 2 kDa or about 5 kDa. The polymer can be linear or branched. The attachment of such polymers (e.g. PEG) adds molecular weight to the construct and may lead to an increased half-life by improving stability, and/or reducing degradation and/or excretion. Conjugation of the polymers may also improve the solubility and stability in aqueous solutions at physiological pH while retaining biological activity of construct. PEG, and any other biological polymers, can be attached to HPPD at any suitable site, e.g., the N- or C-termini, or the side chain of any amino acid which has a functional group suitable for conjugate or which can be synthetically modified.

The above polymers, such as PEG groups, can be attached to the construct under any suitable conditions used to react a protein with an activated polymer molecule. Any means known in the art can be used, including via acylation, reductive alkylation, Michael addition, thiol alkylation or other chemo selective conjugation/ligation methods through a reactive group on the PEG moiety (e.g., an aldehyde, amino, ester, thiol, α-haloacetyl, maleimido or hydrazino group) to a reactive group on the construct (e.g., an aldehyde, amino, ester, thiol, α-haloacetyl, maleimido or hydrazino group). Activating groups which can be used to link the water-soluble polymer to one or more proteins include without limitation sulfone, maleimide, sulfhydryl, thiol, triflate, tresylate, azidirine, oxirane, 5-pyridyl, and alpha-halogenated acyl group (e.g., α-iodo acetic acid, α-bromoacetic acid, α-chloroacetic acid). If attached to the construct by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995).

The construct can be linked to the above polymers via direct covalent linkage by reacting targeted amino acid residues of the peptide with an organic derivatizing agent that is capable of reacting with selected side chains or the N- or C-terminal residues of these targeted amino acids. Reactive groups on the peptide or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, the conjugate moieties can be linked to the construct indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier.

In embodiments, a thiol moiety within the construct is modified with a water-soluble polymer, such as PEG. In some embodiments, the thiol is modified with maleimide-activated PEG in a Michael addition reaction to result in a PEGylated peptide comprising the thioether linkage. In alternative embodiments, a thiol is modified with a haloacetyl-activated PEG in a nucleophilic substitution reaction to result in a PEGylated peptide comprising the thioether linkage. Cysteinyl residues are most commonly reacted with α.-haloacetates (and corresponding amines), such as chloroacetic acid and chloroacetamide, to give carboxymethyl or carboxyamidomethyl derivatives. Cysteinyl residues also are derivatized by reaction with bromotrifluoroacetone, α-bromo-.β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

Carboxyl side groups (aspartyl or glutamyl) are selectively modified by reaction with carbodiimides (R--N.dbd.C.dbd.N--R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Other modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)), deamidation of asparagine or glutamine, acetylation of the N-terminal amine, and/or amidation or esterification of the C-terminal carboxylic acid group.

### Endosomal Escape Vehicles (EEVs)

An endosomal escape vehicle (EEV) can be used to transport a cargo across a cellular membrane, for example, to deliver the cargo to the cytosol or nucleus of a cell. Cargo can include a degradation compound. The EEV can comprise a cell penetrating peptide (CPP), for example, a cyclic cell penetrating peptide (cCPP). In embodiments, the EEV comprises a cCPP, which is conjugated to an exocyclic peptide (EP). The EP can be referred to interchangeably as a modulatory peptide (MP). The EP can comprise a sequence of a nuclear localization signal (NLS). The EP can be coupled to the cargo. The EP can be coupled to the cCPP. The EP can be coupled to the cargo and the cCPP. Coupling between the EP, cargo, cCPP, or combinations thereof, may be non-covalent or covalent. The EP can be attached through a peptide bond to the N-terminus of the cCPP. The EP can be attached through a peptide bond to the C-terminus of the cCPP. The EP can be attached to the cCPP through a side chain of an amino acid in the cCPP. The EP can be attached to the cCPP through a side chain of a lysine which can be conjugated to the side chain of a glutamine in the cCPP. The EP can be conjugated to the 5′ or 3′ end of an oligonucleotide cargo. The EP can be coupled to a linker. The exocyclic peptide can be conjugated to an amino group of the linker. The EP can be coupled to a linker via the C-terminus of an EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to attach the cCPP, the C- or N-terminus may be attached to a linker on the cargo.

Exocyclic Peptides

The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 15 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2, 3, or 4 lysine residues. The amino group on the side chain of each lysine residue can be substituted with a protecting group, including, for example, trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group.

The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH (SEQ ID NO: 1), KHKK (SEQ ID NO: 2), KKHK (SEQ ID NO: 3), KKKH (SEQ ID NO: 4), KHKH (SEQ ID NO: 5), HKHK (SEQ ID NO: 6), KKKK (SEQ ID NO: 7), KKRK (SEQ ID NO: 8), KRKK (SEQ ID NO: 9), KRRK (SEQ ID NO: 10), RKKR (SEQ ID NO: 11), RRRR (SEQ ID NO: 12), KGKK (SEQ ID NO: 13), KKGK (SEQ ID NO: 14), HBHBH (SEQ ID NO: 15), HBKBH (SEQ ID NO: 16), RRRRR (SEQ ID NO: 17), KKKKK (SEQ ID NO: 18), KKKRK (SEQ ID NO: 19), RKKKK (SEQ ID NO: 20), KRKKK (SEQ ID NO: 21), KKRKK (SEQ ID NO: 22), KKKKR (SEQ ID NO: 23), KBKBK (SEQ ID NO: 24), RKKKKG (SEQ ID NO: 25), KRKKKG (SEQ ID NO: 26), KKRKKG (SEQ ID NO: 27), KKKKRG (SEQ ID NO: 28), RKKKKB (SEQ ID NO: 29), KRKKKB (SEQ ID NO: 30), KKRKKB (SEQ ID NO: 31), KKKKRB (SEQ ID NO: 32), KKKRKV (SEQ ID NO: 33), RRRRRR (SEQ ID NO: 34), HHHHHH (SEQ ID NO: 35), RHRHRH (SEQ ID NO: 36), HRHRHR (SEQ ID NO: 37), KRKRKR (SEQ ID NO: 38), RKRKRK (SEQ ID NO: 39), RBRBRB (SEQ ID NO: 40), KBKBKB (SEQ ID NO: 41), PKKKRKV (SEQ ID NO: 42), PGKKRKV (SEQ ID NO: 43), PKGKRKV (SEQ ID NO: 44), PKKGRKV (SEQ ID NO: 45), PKKKGKV (SEQ ID NO: 46), PKKKRGV (SEQ ID NO: 47), or PKKKRKG (SEQ ID NO: 48), wherein B is beta-alanine. The amino acids in the EP can have D or L stereochemistry.

The EP can comprise KK, KR, RR, KKK, KGK, KBK, KBR, KRK, KRR, RKK, RRR, KKKK (SEQ ID NO: 7), KKRK (SEQ ID NO: 8), KRKK (SEQ ID NO: 9), KRRK (SEQ ID NO: 10), RKKR (SEQ ID NO: 11), RRRR (SEQ ID NO: 12), KGKK (SEQ ID NO: 13), KKGK (SEQ ID NO: 14), KKKKK (SEQ ID NO: 18), KKKRK (SEQ ID NO: 19), KBKBK (SEQ ID NO: 24), KKKRKV (SEQ ID NO: 33), PKKKRKV (SEQ ID NO: 42), PGKKRKV (SEQ ID NO: 43), PKGKRKV (SEQ ID NO: 44), PKKGRKV (SEQ ID NO: 45), PKKKGKV (SEQ ID NO: 46), PKKKRGV (SEQ ID NO: 47), or PKKKRKG (SEQ ID NO: 48). The EP can comprise PKKKRKV (SEQ ID NO: 42), RR, RRR, RHR, RBR, RBRBR (SEQ ID NO: 277), RBHBR (SEQ ID NO: 278), or HBRBH (SEQ ID NO: 279), wherein B is beta-alanine. The amino acids in the EP can have D or L stereochemistry.

The EP can consist of KK, KR, RR, KKK, KGK, KBK, KBR, KRK, KRR, RKK, RRR, KKKK (SEQ ID NO: 7), KKRK (SEQ ID NO: 8), KRKK (SEQ ID NO: 9), KRRK (SEQ ID NO: 10), RKKR (SEQ ID NO: 11), RRRR (SEQ ID NO: 12), KGKK (SEQ ID NO: 13), KKGK (SEQ ID NO: 14), KKKKK (SEQ ID NO: 18), KKKRK (SEQ ID NO: 19), KBKBK (SEQ ID NO: 24), KKKRKV (SEQ ID NO: 33), PKKKRKV (SEQ ID NO: 42), PGKKRKV (SEQ ID NO: 43), PKGKRKV (SEQ ID NO: 44), PKKGRKV (SEQ ID NO: 45), PKKKGKV (SEQ ID NO: 46), PKKKRGV (SEQ ID NO: 47), or PKKKRKG (SEQ ID NO: 48). The EP can consist of PKKKRKV (SEQ ID NO: 42), RR, RRR, RHR, RBR, RBRBR (SEQ ID NO: 277), RBHBR (SEQ ID NO: 278), or HBRBH (SEQ ID NO: 279), wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV (SEQ ID NO: 42). The EP can consist of an NLS comprising the amino acid sequence PKKKRKV (SEQ ID NO: 42). The EP can comprise an NLS comprising an amino acid sequence selected from NLSKRPAAIKKAGQAKKKK (SEQ ID NO: 280), PAAKRVKLD (SEQ ID NO: 281), RQRRNELKRSF (SEQ ID NO: 282), RMRKFKNKGKDTAELRRRRVEVSVELR (SEQ ID NO: 283), KAKKDEQILKRRNV (SEQ ID NO: 284), VSRKRPRP (SEQ ID NO: 285), PPKKARED (SEQ ID NO: 286), PQPKKKPL (SEQ ID NO: 287), SALIKKKKKMAP (SEQ ID NO: 288), DRLRR (SEQ ID NO: 289), PKQKKRK (SEQ ID NO: 290), RKLKKKIKKL (SEQ ID NO: 291), REKKKFLKRR (SEQ ID NO: 292), KRKGDEVDGVDEVAKKKSKK (SEQ ID NO: 293), and RKCLQAGMNLEARKTKK (SEQ ID NO: 294). The EP can consist of an NLS comprising an amino acid sequence selected from NLSKRPAAIKKAGQAKKKK (SEQ ID NO: 280), PAAKRVKLD (SEQ ID NO: 281), RQRRNELKRSF (SEQ ID NO: 282), RMRKFKNKGKDTAELRRRRVEVSVELR (SEQ ID NO: 283), KAKKDEQILKRRNV (SEQ ID NO: 284), VSRKRPRP (SEQ ID NO: 285), PPKKARED (SEQ ID NO: 286), PQPKKKPL (SEQ ID NO: 287), SALIKKKKKMAP (SEQ ID NO: 288), DRLRR (SEQ ID NO: 289), PKQKKRK (SEQ ID NO: 290), RKLKKKIKKL (SEQ ID NO: 291), REKKKFLKRR (SEQ ID NO: 292), KRKGDEVDGVDEVAKKKSKK (SEQ ID NO: 293), and RKCLQAGMNLEARKTKK (SEQ ID NO: 294).

All exocyclic sequences can also contain an N-terminal acetyl group. Hence, for example, the EP can have the structure: Ac-PKKKRKV (SEQ ID NO: 295).

Cell Penetrating Peptides (CPP)

The cell penetrating peptide (CPP) can comprise 6 to 20 amino acid residues. The cell penetrating peptide can be a cyclic cell penetrating peptide (cCPP). The cCPP is capable of penetrating a cell membrane. An exocyclic peptide (EP) can be conjugated to the cCPP, and the resulting construct can be referred to as an endosomal escape vehicle (EEV). The cCPP can direct a cargo (e.g., a therapeutic moiety TM) such as an oligonucleotide, peptide or small molecule) to penetrate the membrane of a cell. The cCPP can deliver the cargo to the cytosol of the cell. The cCPP can deliver the cargo to a cellular location where a target (e.g., pre-mRNA) is located. To conjugate the cCPP to a cargo (e.g., peptide, oligonucleotide, or small molecule), at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP disclosed herein can comprise 6 to 10 amino acids. By way of example, cCPP comprising 6-10 amino acid residues can have a structure according to any of Formula I-A to I-E:

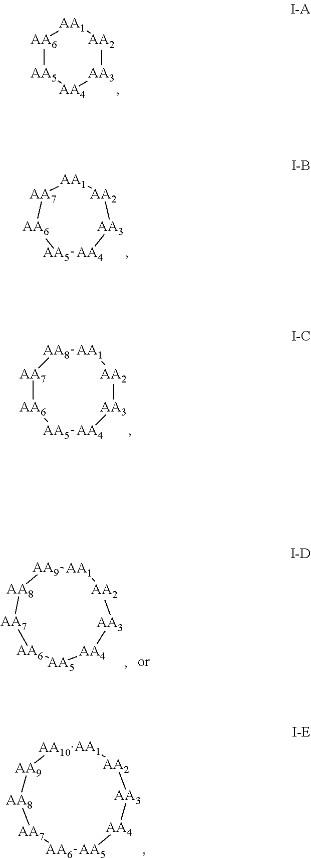

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 13 along with their abbreviations used herein.

TABLE 13
Amino Acid Abbreviations

| | Abbreviations* | Abbreviations* |
|---|---|---|
| Amino Acid | L-amino acid | D-amino acid |
| 2-[2-[2-aminoethoxy] | AEEA, | NA |
| ethoxy]acetic acid | miniPEG | |
| Alanine | Ala (A) | ala (a) |
| Allo-isoleucine | Aile | Aile |
| Arginine | Arg (R) | arg (r) |
| Asparagine | Asn (N) | asn (n) |
| aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| glutamic acid | Glu (E) | glu (e) |
| glutamine | Gln (Q) | gln (q) |
| glycine | Gly (G) | gly (g) |
| histidine | His (H) | his (h) |
| Homoproline (aka | Pip (Θ) | Pip (θ) |
| pipecolic acid) | | |
| isoleucine | Ile (I) | ile (i) |
| leucine | Leu (L) | leu (l) |
| lysine | Lys (K) | lys (k) |
| methionine | Met (M) | met (m) |
| 3-(2-naphthyl)-alanine | Nal (Φ) | nal (ϕ) |
| 3-(1-naphthyl)-alanine | 1-Nal | 1-nal |
| norleucine | Nle (Ω) | nle |
| phenylalanine | Phe (F) | Phe (f) |
| phenylglycine | Phg (Ψ) | phg |
| 4-(phosphonodifluoro- | F₂Pmp (∧) | f₂pmp |
| methyl)phenylalanine | | |
| proline | Pro (P) | pro (p) |
| sarcosine | Sar(≡) | sar |
| selenocysteine | Sec(U) | sec (u) |
| serine | Ser (S) | ser (s) |
| threonine | Thr (T) | thr (y) |
| tyrosine | Tyr(Y) | tyr (y) |
| tryptophan | Trp(W) | trp (w) |
| valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| T riflouroacetyl-lysine | Lys(TFA) | lys(TFA) |
| Methyl-leucine | MeLeu | meLeu |
| 3-(3-benzothienyl)-alanine | Bta | bta |

*single letter abbreviations: capital letters indicate the L-amino acid form, lower case letter indicate the D-amino acid form.

As used herein, “polyethylene glycol” and “PEG” are used interchangeably. “PEGm,” and “PEGₘ,” are, or are derived from, a molecule of the formula HO(CO)—(CH₂)ₙ—(OCH₂CH₂)ₘ—NH₂ where n is any integer from 1 to 5 and m is any integer from 1 to 23. In embodiments, n is 1 or 2. In embodiments, n is 1. In embodiments, n is 2. In embodiments, n is 1 and m is 2. In embodiments, n is 2 and m is 2. In embodiments, n is 1 and m is 4. In embodiments, n is 2 and m is 4. In embodiments, n is 1 and m is 12. In embodiments, n is 2 and m is 12.

As used herein, “miniPEGm” or “miniPEGₘ” are, or are derived from, a molecule of the formula HO(CO)—(CH₂)ₙ—(OCH₂CH₂)ₘ—NH₂ where n is 1 and m is any integer from 1 to 23. For example, “miniPEG2” or “miniPEG₂” is, or is derived from, (2-[2-[2-aminoethoxy]ethoxy]acetic acid), and “miniPEG4” or “miniPEG₄” is, or is derived from, HO(CO)—(CH₂)ₙ—(OCH₂CH₂)ₘ—NH₂ where n is 1 and m is 4.

The cCPP can comprise 4 to 20 amino acids, wherein: (i) at least one amino acid has a side chain comprising a guanidine group, or a protonated form thereof; (ii) at least one amino acid has no side chain or a side chain

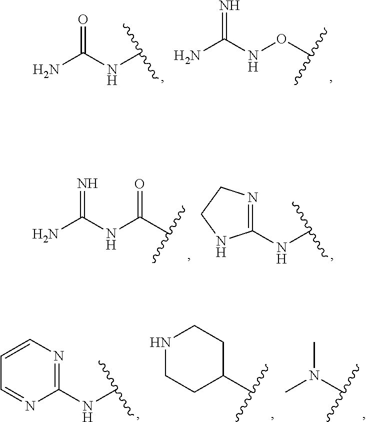

or a protonated form thereof; and (iii) at least two amino acids independently have a side chain comprising an aromatic or heteroaromatic group.

At least two amino acids can have no side chain or a side chain comprising

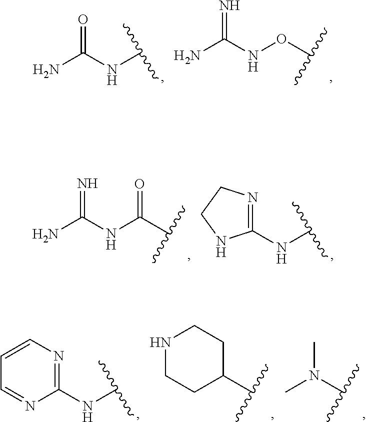

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or β-alanine.

The cCPP can comprise from 6 to 20 amino acid residues which form the cCPP, wherein: (i) at least one amino acid can be glycine, β-alanine, or 4-aminobutyric acid residues; (ii) at least one amino acid can have a side chain comprising an aryl or heteroaryl group; and (iii) at least one amino acid has a side chain comprising a guanidine group,

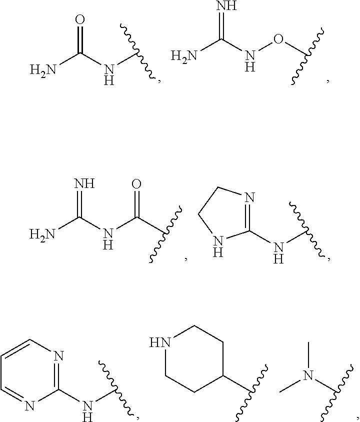

or a protonated form thereof.

The cCPP can comprise from 6 to 20 amino acid residues which form the cCPP, wherein: (i) at least two amino acid can independently beglycine, β-alanine, or 4-aminobutyric acid residues; (ii) at least one amino acid can have a side chain comprising an aryl or heteroaryl group; and (iii) at least one amino acid has a side chain comprising a guanidine group,

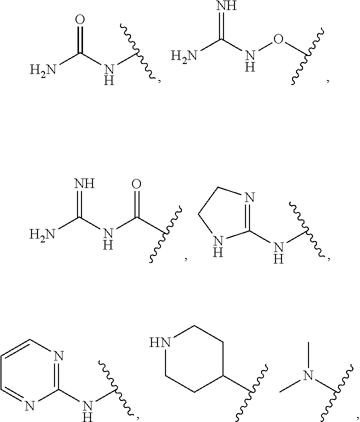

a protonated form thereof.

The cCPP can comprise from 6 to 20 amino acid residues which form the cCPP, wherein: (i) at least three amino acids can independently be glycine, β-alanine, or 4-aminobutyric acid residues; (ii) at least one amino acid can have a side chain comprising an aromatic or heteroaromatic group; and (iii) at least one amino acid can have a side chain comprising a guanidine group,

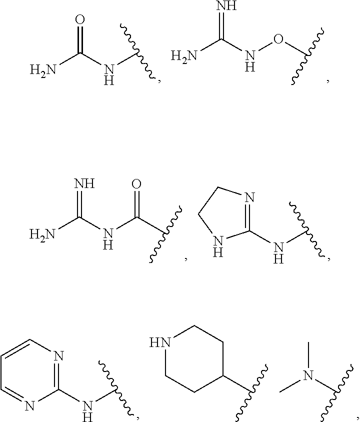

or a protonated form thereof.

Glycine and Related Amino Acid Residues

The cCPP can comprise (i) 1, 2, 3, 4, 5, or 6 glycine, β-alanine, 4-aminobutyric acid residues, or combinations thereof. The cCPP can comprise (i) 2 glycine, β-alanine, 4-aminobutyric acid residues, or combinations thereof. The cCPP can comprise (i) 3 glycine, (3-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (i) 3, 4, 5, or 6 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues

In embodiments, none of the glycine, β-alanine, or 4-aminobutyric acid residues in the cCPP are contiguous. Two or three glycine, β-alanine, 4- or aminobutyric acid residues can be contiguous. Two glycine, β-alanine, or 4-aminobutyric acid residues can be contiguous.

In embodiments, none of the glycine residues in the cCPP are contiguous. Each glycine residues in the cCPP can be separated by an amino acid residue that cannot be glycine. Two or three glycine residues can be contiguous. Two glycine residues can be contiguous.

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group. The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

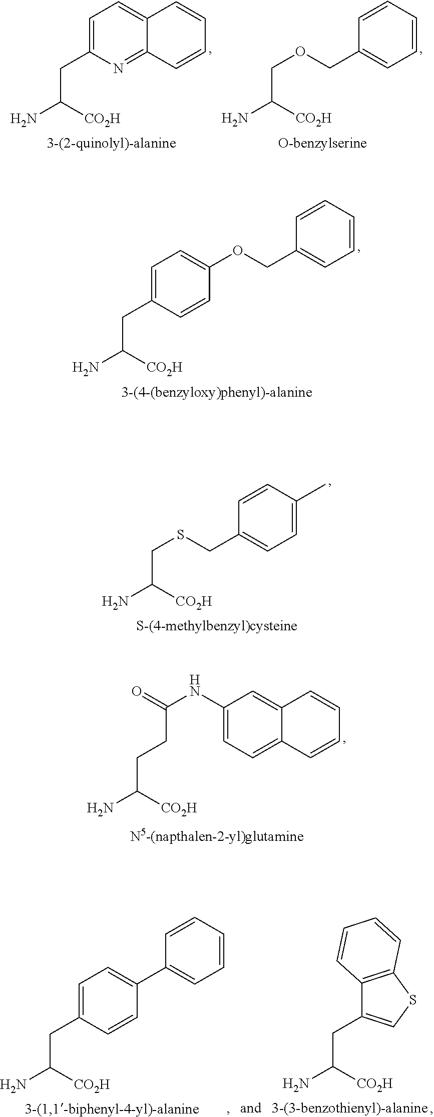

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, 0-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

While not wishing to be bound by theory, it is believed that amino acids having an aromatic or heteroaromatic group having higher hydrophobicity values (i.e., amino acids having side chains comprising aromatic or heteroaromatic groups) can improve cytosolic delivery efficiency of a cCPP relative to amino acids having a lower hydrophobicity value. Each hydrophobic amino acid can independently have a hydrophobicity value greater than that of glycine. Each hydrophobic amino acid can independently be a hydrophobic amino acid having a hydrophobicity value greater than that of alanine. Each hydrophobic amino acid can independently have a hydrophobicity value greater or equal to phenylalanine. Hydrophobicity may be measured using hydrophobicity scales known in the art. Table 14 lists hydrophobicity values for various amino acids as reported by Eisenberg and Weiss (Proc. Natl. Acad. Sci. U.S.A. 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A 1981; 78(6):3824-3828), and Janin (Nature. 1979; 277(5696):491-492), the entirety of each of which is herein incorporated by reference. Hydrophobicity can be measured using the hydrophobicity scale reported in Engleman, et al.

TABLE 14
Amino Acid Hydrophobicity

| | | | | Kyrie | Hoop | |
|---|---|---|---|---|---|---|
| Amino | | Eisenberg | Engleman | and | and | |
| Acid | Group | and Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gin | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

The size of the aromatic or heteroaromatic groups may be selected to improve cytosolic delivery efficiency of the cCPP. While not wishing to be bound by theory, it is believed that a larger aromatic or heteroaromatic group on the side chain of amino acid may improve cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. The size of the hydrophobic amino acid can be measured in terms of the molecular weight of the hydrophobic amino acid, and the larger hydrophobic amino acid has a side chain with a molecular weight of at least about 90 g/mol, or at least about 130 g/mol, or at least about 141 g/mol. The size of the amino acid can be measured in terms of the SASA of the hydrophobic side chain. The hydrophobic amino acid can have a side chain with a SASA of greater than or equal to alanine, or greater than or equal to glycine. Larger hydrophobic amino acids can have a side chain with a SASA greater than alanine, or greater than glycine. The hydrophobic amino acid can have an aromatic or heteroaromatic group with a SASA greater than or equal to about piperidine-2-carboxylic acid, greater than or equal to about tryptophan, greater than or equal to about phenylalanine, or greater than or equal to about naphthylalanine. A first hydrophobic amino acid (AA_{H1}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². A second hydrophobic amino acid (AA_{H2}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². The side chains of AA_{H1} and AA_{H2} can have a combined SASA of at least about 350 Å², at least about 360 Å², at least about 370 Å², at least about 380 Å², at least about 390 Å², at least about 400 Å², at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². AA_{H2} can be a hydrophobic amino acid residue with a side chain having a SASA that is less than or equal to the SASA of the hydrophobic side chain of AA_{H1}. By way of example, and not by limitation, a cCPP having a Nal-Arg motif may exhibit improved cytosolic delivery efficiency compared to an otherwise identical cCPP having a Phe-Arg motif, a cCPP having a Phe-Nal-Arg motif may exhibit improved cytosolic delivery efficiency compared to an otherwise identical cCPP having a Nal-Phe-Arg motif, and a phe-Nal-Arg motif may exhibit improved cytosolic delivery efficiency compared to an otherwise identical cCPP having a nal-Phe-Arg motif.

As used herein, “hydrophobic surface area” or “SASA” refers to the surface area (reported as square Ångstroms; Å²) of an amino acid side chain that is accessible to a solvent. SASA can be calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 15. The SASA values described herein are based on the theoretical values listed in Table 15 below, as reported by Tien, et al. (PLOS ONE 8(11): e80635, available at doi.org/10.1371/journal.pone.0080635), which is herein incorporated by reference in its entirety for all purposes.

TABLE 15
Amino Acid SASA Values

| | | | Miller et | Rose et |
|---|---|---|---|---|
| Residue | Theoretical | Empirical | al. (1987) | al. (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

Amino Acid Residues Having a Side Chain Comprising a Guanidine Group, Guanidine Replacement Group, or Protonated Form Thereof

As used herein, guanidine refers to the structure:

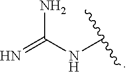 [SMILES: *NC(=N)N]

As used herein, a protonated form of guanidine refers to the structure:

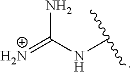 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

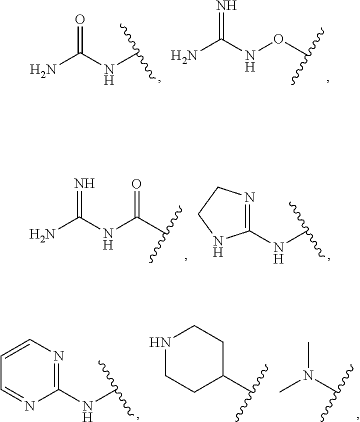

The disclosure relates to a cCPP comprising from 4 to 20 amino acids residues, wherein: (i) at least one amino acid has a side chain comprising a guanidine group, or a protonated form thereof, (ii) at least one amino acid residue has no side chain or a side chain comprising

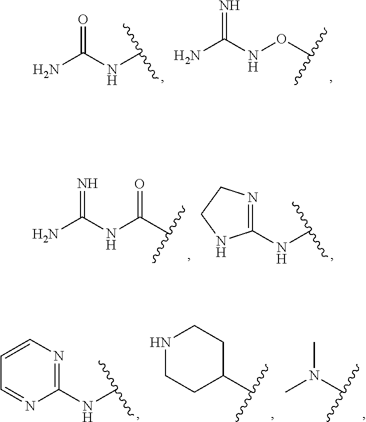

or a protonated form thereof; and (iii) at least two amino acids residues independently have a side chain comprising an aromatic or heteroaromatic group.

At least two amino acids residues can have no side chain or a side chain comprising

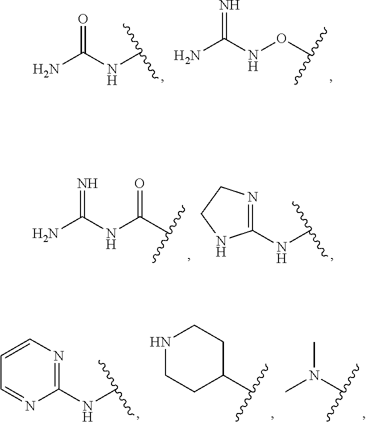

a protonated form thereof. As used herein, when no side chain is present, the amino acid residue have two hydrogen atoms on the carbon atom(s) (e.g., —CH₂—) linking the amine and carboxylic acid.

The cCPP can comprise at least one amino acid having a side chain comprising one of the following moieties:

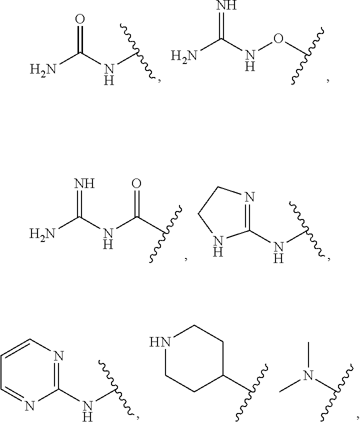

or a protonated form thereof.

The cCPP can comprise at least two amino acids each independently having one of the following moieties

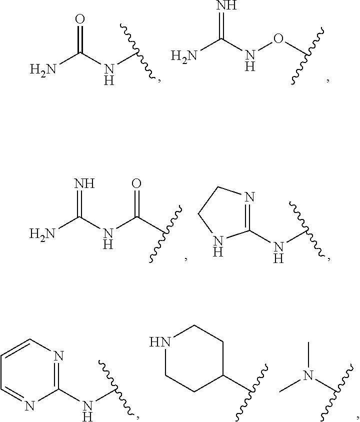

or a protonated form thereof. At least two amino acids can have a side chain comprising the same moiety selected from:

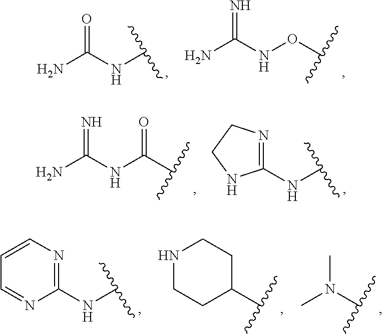

or a protonated form thereof. At least one amino acid can have a side chain comprising

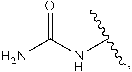 [SMILES: *NC(N)=O]

or a protonated form thereof. At least two amino acids can have a side chain comprising

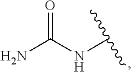 [SMILES: *NC(N)=O]

or a protonated form thereof. One, two, three, or four amino acids can have a side chain comprising

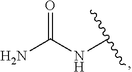 [SMILES: *NC(N)=O]

or a protonated form thereof. One amino acid can have a side chain comprising

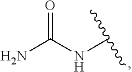 [SMILES: *NC(N)=O]

or a protonated form thereof. Two amino acids can have a side chain comprising

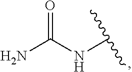 [SMILES: *NC(N)=O]

or a protonated form thereof.

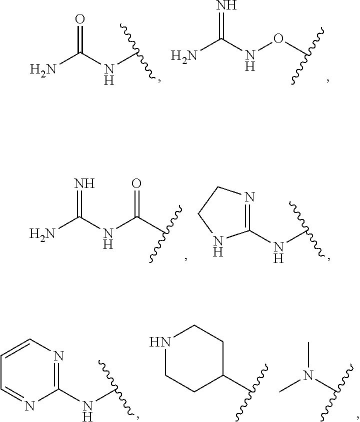

or a protonated form thereof, can be attached to the terminus of the amino acid side chain.

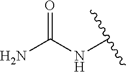 [SMILES: *NC(N)=O]

can be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently

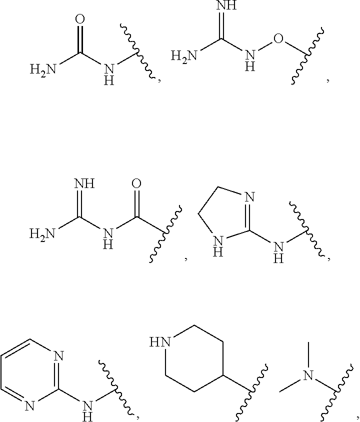

or a protonated form thereof.

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutamic acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids. The amino acid residues that form the cCPP can all be D-amino acids.

At least two of the amino acids can have a different chirality. At least two amino acids having a different chirality can be adjacent to each other. At least three amino acids can have different chirality relative to an adjacent amino acid. At least four amino acids can have different chirality relative to an adjacent amino acid. At least two amino acids have the same chirality and at least two amino acids have a different chirality. One or more amino acid residues that form the cCPP can be achiral. The cCPP can comprise a motif of 3, 4, or 5 amino acids, wherein two amino acids having the same chirality can be separated by an achiral amino acid. The cCPPs can comprise the following sequences: D-X-D; D-X-D-X; D-X-D-X-D; L-X-L; L-X-L-X; or L-X-L-X-L, wherein X is an achiral amino acid. The achiral amino acid can be glycine.

An amino acid having a side chain comprising:

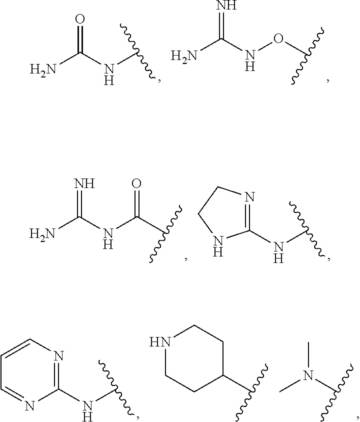

or a protonated form thereof, can be adjacent to an amino acid having a side chain comprising an aromatic or heteroaromatic group. An amino acid having a side chain comprising:

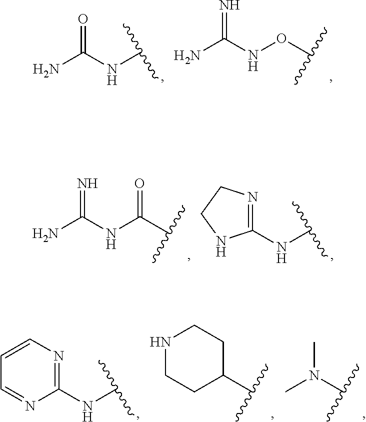

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

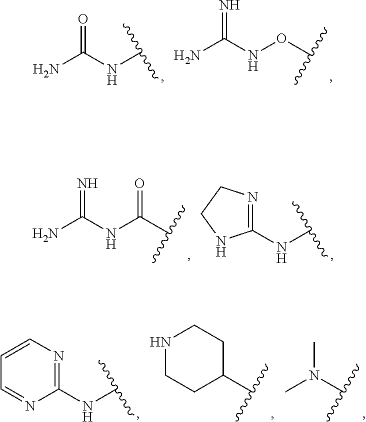

or protonated forms thereof, can be adjacent to each other. Two amino acids having a side chain comprising a guanidine or protonated form thereof are adjacent to each other.

The cCPPs can comprise at least two contiguous amino acids having a side chain can comprise an aromatic or heteroaromatic group and at least two non-adjacent amino acids having a side chain comprising

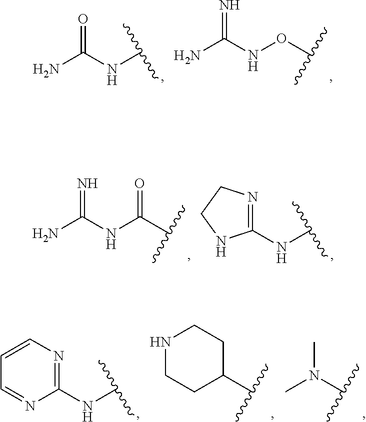

or a protonated form thereof. The cCPPs can comprise at least two contiguous amino acids having a side chain comprising an aromatic or heteroaromatic group and at least two non-adjacent amino acids having a side chain comprising

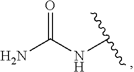 [SMILES: *NC(N)=O]

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

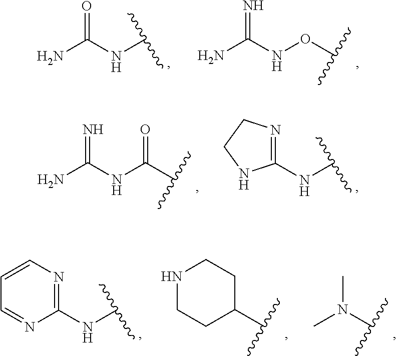

or a protonated form thereof, are alternating with at least two amino acids having a side chain comprising a guanidine group or protonated form thereof.

The cCPP can comprise the structure of Formula (A):

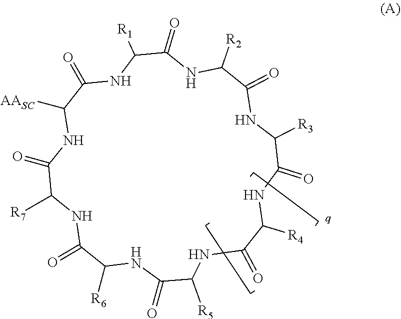

or a protonated form thereof,

wherein:
R₁, R₂, and R₃ are each independently H or an aromatic or heteroaromatic side chain of an amino acid;

at least one of R₁, R₂, and R₃ is an aromatic or heteroaromatic side chain of an amino acid;

R₄, R₅, R₆, R₇ are independently H or an amino acid side chain; at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;

AA_{SC} is an amino acid side chain; and

q is 1, 2, 3 or 4.

In embodiments, the cyclic peptide of Formula (A) is not FfΦRrRrQ. In embodiments, the cyclic peptide of Formula (A) is FfΦRrRrQ.

The cCPP can comprise the structure of Formula (I):

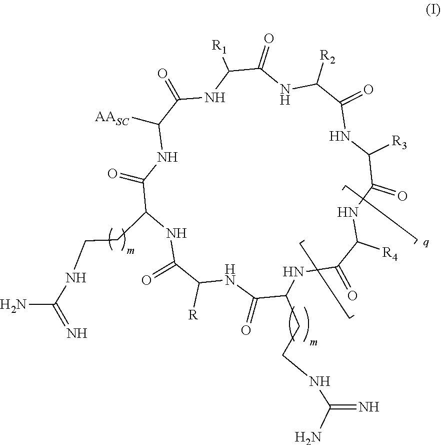

or a protonated form thereof,

wherein:
R₁, R₂, and R₃ can each independently be H or an amino acid residue having a side chain comprising an aromatic group;

at least one of R₁, R₂, and R₃ is an aromatic or heteroaromatic side chain of an amino acid;

R₄ and R₇ are independently H or an amino acid side chain;

AA_{SC} is an amino acid side chain;

q is 1, 2, 3 or 4; and

each m is independently an integer of 0, 1, 2, or 3.

R₁, R₂, and R₃ can each independently be H, -alkylene-aryl, or -alkylene-heteroaryl. R₁, R₂, and R₃ can each independently be H, —C₁₋₃alkylene-aryl, or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂, and R₃ can each independently be H, —CH₂Ph, or —CH₂Naphthyl. R₁, R₂, and R₃ can each independently be H or —CH₂Ph.

R₁, R₂, and R₃ can each independently be the side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ can be the side chain of tyrosine. R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of 3-benzothienylalanine. R₁ can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of β-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

R₂ can be the side chain of tyrosine. R₂ can be the side chain of phenylalanine. R₂ can be the side chain of 1-naphthylalanine. R₁ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of β-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

R₃ can be the side chain of tyrosine. R₃ can be the side chain of phenylalanine. R₃ can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. R₃ can be the side chain of 4-trifluoromethylphenylalanine. R₃ can be the side chain of 2,3,4,5,6-pentafluorophenylalanine. R₃ can be the side chain of homophenylalanine. R₃ can be the side chain of β-homophenylalanine. R₃ can be the side chain of 4-tert-butyl-phenylalanine. R₃ can be the side chain of 4-pyridinylalanine. R₃ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

R₄ can be H, -alkylene-aryl, -alkylene-heteroaryl. R₄ can be H, —C₁₋₃alkylene-aryl, or —C₁₋₃ alkylene-heteroaryl. R₄ can be H or -alkylene-aryl. R₄ can be H or —C₁₋₃alkylene-aryl. C₁₋₃ alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₄ can be H, —C₁₋₃alkylene-Ph or —C₁₋₃alkylene-Naphthyl. R₄ can be H or the side chain of an amino acid in Table 15 or Table 17. R₄ can be H or an amino acid residue having a side chain comprising an aromatic group. R₄ can be H, —CH₂Ph, or —CH₂Naphthyl. R₄ can be H or —CH₂Ph.

R₅ can be H, -alkylene-aryl, -alkylene-heteroaryl. R₅ can be H, —C₁₋₃alkylene-aryl, or —C₁₋₃ alkylene-heteroaryl. R₅ can be H or -alkylene-aryl. R₅ can be H or —C₁₋₃alkylene-aryl. C₁₋₃ alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₅ can be H, —C₁₋₃alkylene-Ph or —C₁₋₃alkylene-Naphthyl. R₅ can be H or the side chain of an amino acid in Table 13 or Table 15. R₄ can be H or an amino acid residue having a side chain comprising an aromatic group. R₅ can be H, —CH₂Ph, or —CH₂Naphthyl. R₄ can be H or —CH₂Ph.

R₆ can be H, -alkylene-aryl, -alkylene-heteroaryl. R₆ can be H, —C₁₋₃alkylene-aryl, or —C₁₋₃ alkylene-heteroaryl. R₆ can be H or -alkylene-aryl. R₆ can be H or —C₁₋₃alkylene-aryl. C₁₋₃ alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₆ can be H, —C₁₋₃alkylene-Ph or —C₁₋₃alkylene-Naphthyl. R₆ can be H or the side chain of an amino acid in Table 13 or Table 15. R₆ can be H or an amino acid residue having a side chain comprising an aromatic group. R₆ can be H, —CH₂Ph, or —CH₂Naphthyl. R₆ can be H or —CH₂Ph.

R₇ can be H, -alkylene-aryl, -alkylene-heteroaryl. R₇ can be H, —C₁₋₃alkylene-aryl, or —C₁₋₃ alkylene-heteroaryl. R₇ can be H or -alkylene-aryl. R₇ can be H or —C₁₋₃alkylene-aryl. C₁₋₃ alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₇ can be H, —C₁₋₃alkylene-Ph or —C₁₋₃alkylene-Naphthyl. R₇ can be H or the side chain of an amino acid in Table 13 or Table 15. R₇ can be H or an amino acid residue having a side chain comprising an aromatic group. R₇ can be H, —CH₂Ph, or —CH₂Naphthyl. R₇ can be H or —CH₂Ph.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₄, R₅, R₆, and R₇ can be —CH₂Ph. Three of R₁, R₂, R₃, R₄, R₅, R₆, and R₇ can be —CH₂Ph. At least one of R₁, R₂, R₃, R₄, R₅, R₆, and R₇ can be —CH₂Ph. No more than four of R₁, R₂, R₃, R₄, R₅, R₆, and R₇ can be —CH₂Ph.

One, two or three of R₁, R₂, R₃, and R₄ are —CH₂Ph. One of R₁, R₂, R₃, and R₄ is —CH₂Ph. Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₅, R₆, and R₇ can be H. One of R₁, R₂, R₃, R₄, R₅, R₆, and R₇ can be H. Two of R₁, R₂, R₃, R₄, R₅, R₆, and R₇ are H. Three of R₁, R₂, R₃, R₅, R₆, and R₇ can be H. At least one of R₁, R₂, R₃, R₄, R₅, R₆, and R₇ can be H. No more than three of R₁, R₂, R₃, R₄, R₅, R₆, and R₇ can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

At least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid. At least one of R₄, R₅, R₆, and R₇ can be side chain of arginine. At least one of R₄, R₅, R₆, and R₇ can be side chain of homoarginine. At least one of R₄, R₅, R₆, and R₇ can be side chain of N-methylarginine. At least one of R₄, R₅, R₆, and R₇ can be side chain of N,N-dimethylarginine. At least one of R₄, R₅, R₆, and R₇ can be side chain of 2,3-diaminopropionic acid. At least one of R₄, R₅, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R₇ can be side chain of N,N-dimethyllysine, β-homoarginine. At least one of R₄, R₅, R₆, and R₇ can be side chain of 3-(1-piperidinyl)alanine.

At least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid. At least two of R₄, R₅, R₆, and R₇ can be side chain of arginine. At least two of R₄, R₅, R₆, and R₇ can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₅, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, β-homoarginine. At least two of R₄, R₅, R₆, and R₇ can be side chain of 3-(1-piperidinyl)alanine.

At least three of R₄, R₅, R₆, and R₇ can be side chain of 3-guanidino-2-aminopropionic acid. At least three of R₄, R₅, R₆, and R₇ can be side chain of 4-guanidino-2-aminobutanoic acid. At least three of R₄, R₅, R₆, and R₇ can be side chain of arginine. At least three of R₄, R₅, R₆, and R₇ can be side chain of homoarginine. At least three of R₄, R₅, R₆, and R₇ can be side chain of N-methylarginine. At least three of R₄, R₅, R₆, and R₇ can be side chain of N,N-dimethylarginine. At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R₇ can be side chain of N-ethyllysine. At least three of R₄, R₅, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N,N-dimethyllysine, β-homoarginine. At least three of R₄, R₅, R₆, and R₇ can be side chain of 3-(1-piperidinyl)alanine.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

q can be 1, 2, or 3. q can 1 or 2. q can be 1. q can be 2. q can be 3. q can be 4.

m can be 1-3. m can be 1 or 2. m can be 0. m can be 1. m can be 2. m can be 3.

The cCPP of Formula (A) can comprise the structure of Formula (I)

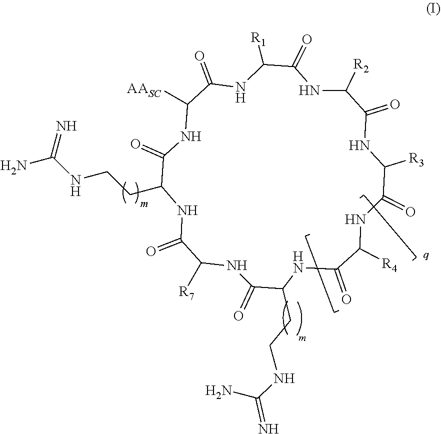

or protonated form thereof, wherein AA_{SC}, R₁, R₂, R₃, R₄, R₇, m, and q are as defined herein.

The cCPP of Formula (A) can comprise the structure of Formula (I-a) or Formula (I-b):

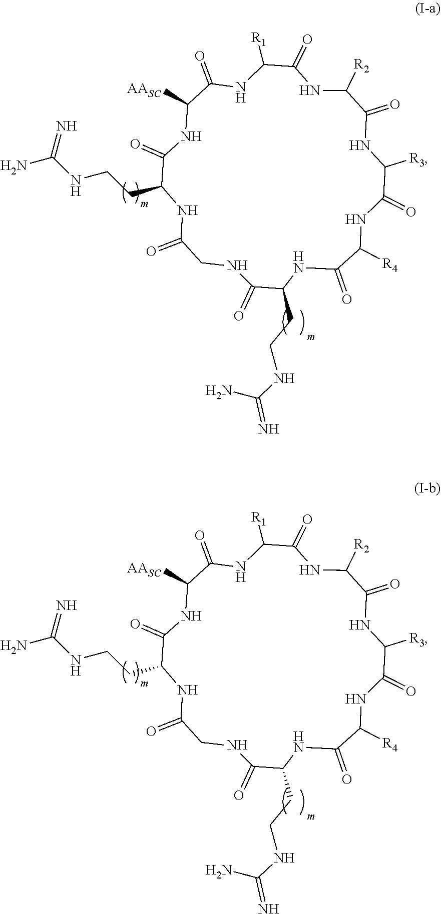

or protonated form thereof, wherein AA_{SC}, R₁, R₂, R₃, R₄, and m are as defined herein.

The cCPP of Formula (A) can comprise the structure of Formula (I-1), (I-2), (I-3), or (I-4):

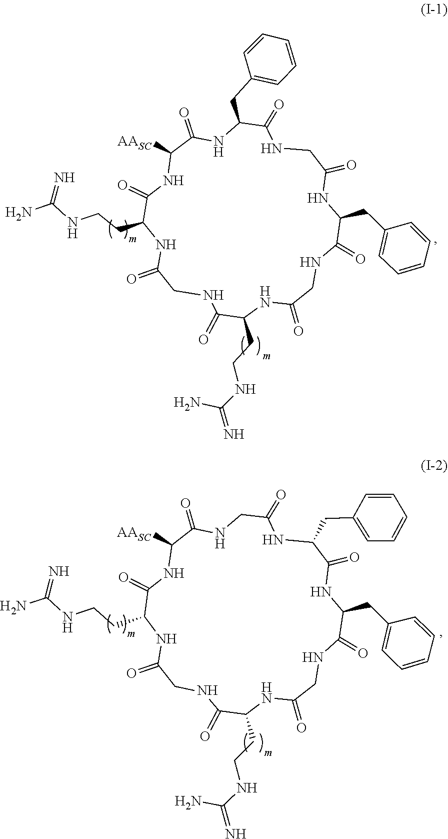

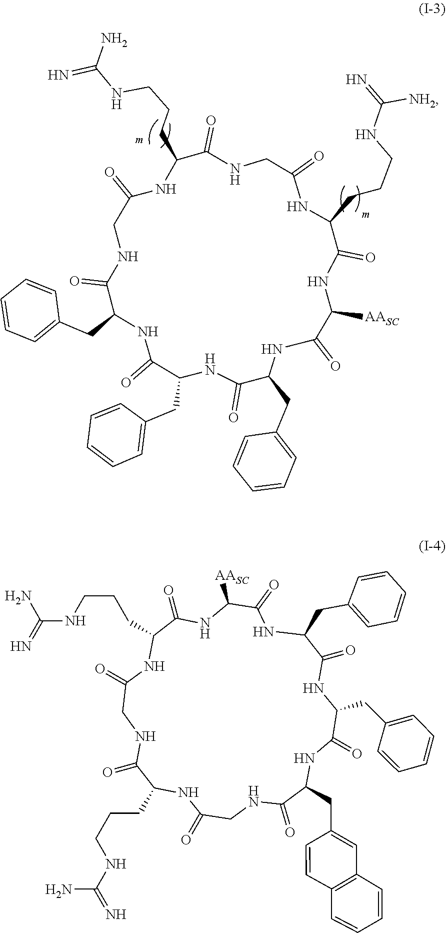

or protonated form thereof, wherein AA_{SC} and m are as defined herein.

The cCPP of Formula (A) can comprise the structure of Formula (I-5) or (I-6):

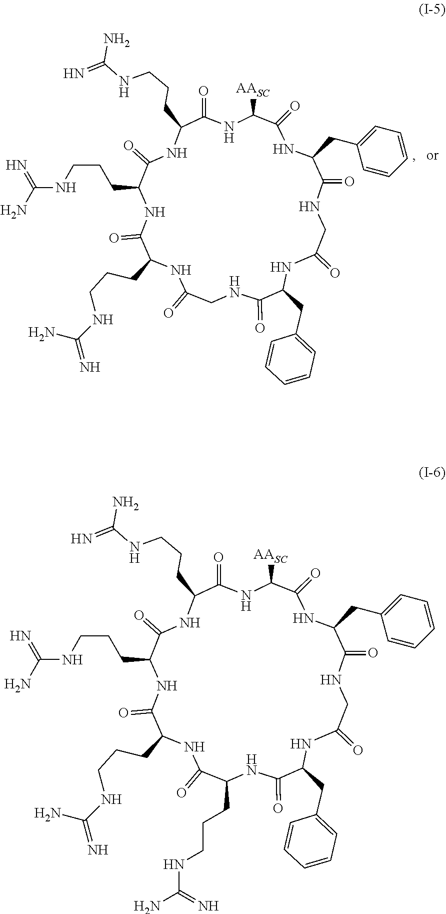

or protonated form thereof, wherein AA_{SC} is as defined herein.

The cCPP of Formula (A) can comprise the structure of Formula (I-1):

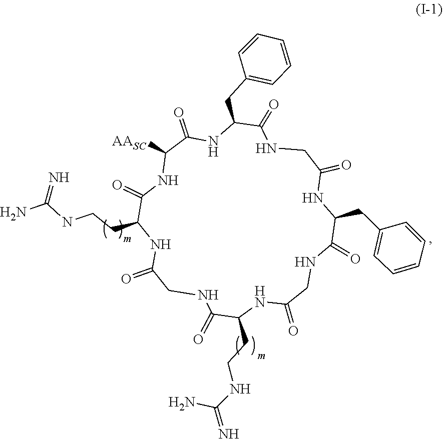

or a protonated form thereof,

wherein AA_{SC} and m are as defined herein.

The cCPP of Formula (A) can comprise the structure of Formula (I-2):

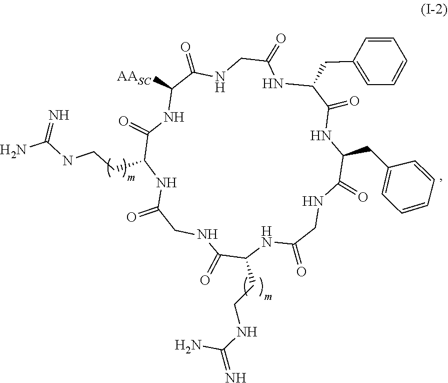

or a protonated form thereof,

wherein AA_{SC} and m are as defined herein.

The cCPP of Formula (A) can comprise the structure of Formula (I-3):

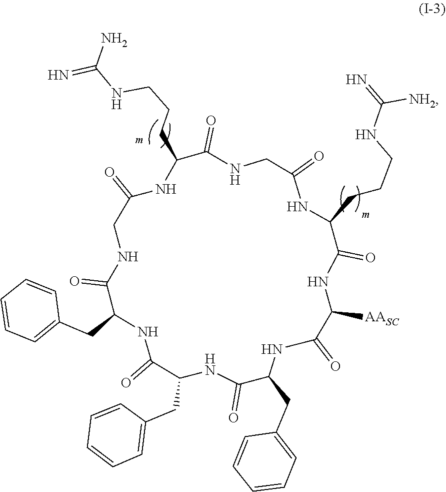

or a protonated form thereof,

wherein AA_{SC} and m are as defined herein.

The cCPP of Formula (A) can comprise the structure of Formula (I-4):

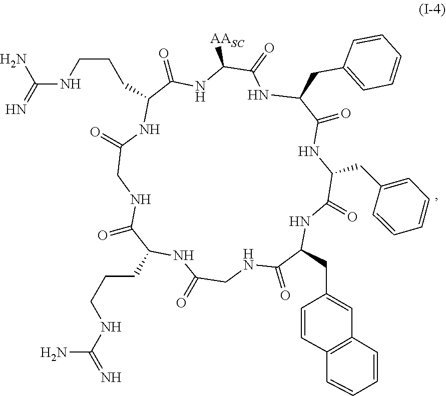

), or a protonated form thereof, wherein AA_{SC} and m are as defined herein.

The cCPP of Formula (A) can comprise the structure of Formula (I-5):

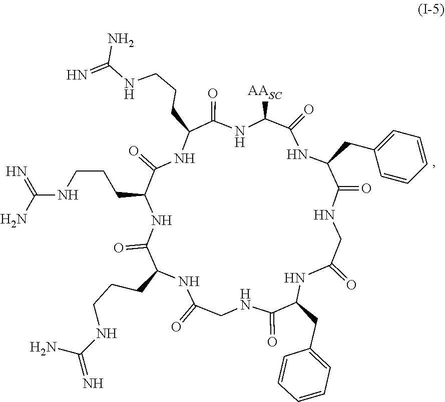

or a protonated form thereof, wherein AA_{SC} and m are as defined herein.

The cCPP of Formula (A) can comprise the structure of Formula (I-6):

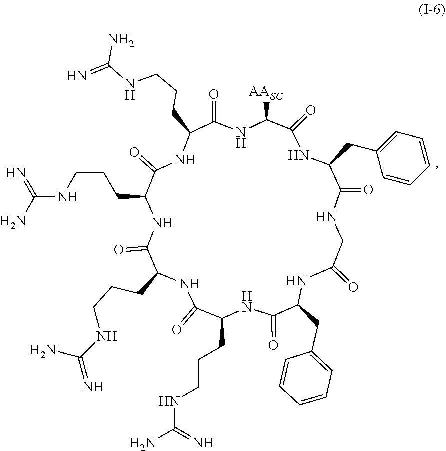

or a protonated form thereof, wherein AA_{SC} and m are as defined herein.

The cCPP can comprise one of the following sequences: FGFGRGR (SEQ ID NO: 296); GfFGrGr, FfΦGRGR; FfFGRGR; or FfΦGrGr. The cCPP can have one of the following sequences: FGFΦ (SEQ ID NO: 297); GfFGrGrQ, FfΦGRGRQ, FfFGRGRQ; or FfΦGrGrQ.

The disclosure also relates to a cCPP having the structure of Formula (II):

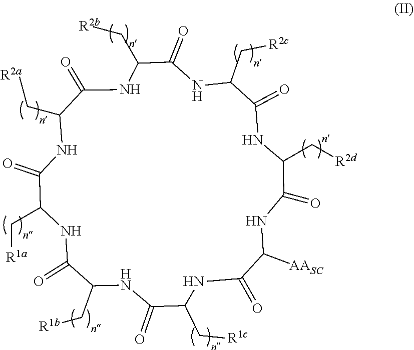

wherein:

AA_{SC} is an amino acid side chain;

R^{1a}, R^{1b}, and R^{1c} are each independently a 6- to 14-membered aryl or a 6- to 14-membered heteroaryl;

R^{2a}, R^{2b}, R^{2c} and R^{2d} are independently an amino acid side chain;

at least one of R^{2a}, R^{2b}, R^{2c} and R^{2d} is

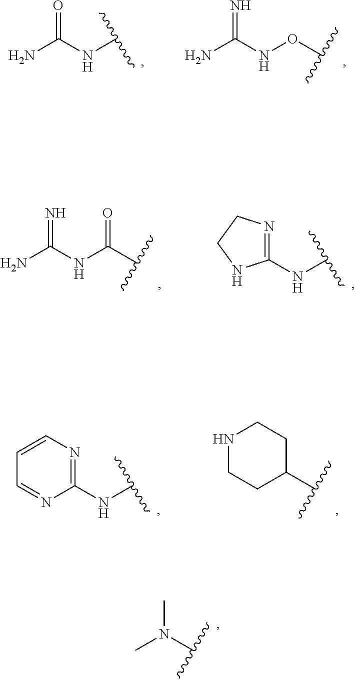

or a protonated form thereof,

at least one of R^{2a}, R^{2b}, R^{2c} and R^{2d} is guanidine or a protonated form thereof;

each n″ is independently an integer 0, 1, 2, 3, 4, or 5;

each n′ is independently an integer from 0, 1, 2, or 3; and

if n′ is 0 then R^{2a}, R^{2b}, R^{2c} or R^{2d} is absent.

At least two of R^{2a}, R^{2b}, R^{2c} and R^{2d} can be

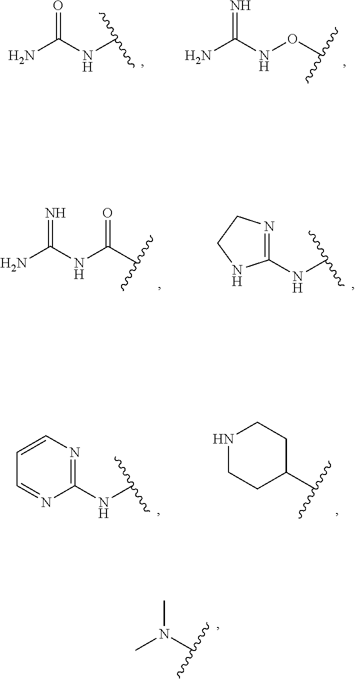

or a protonated form thereof. Two or three of R^{2a}, R^{2b}, R^{2c} and R^{2d} can be

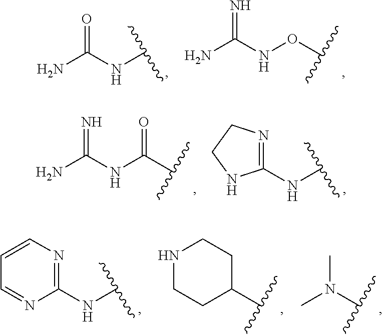

or a protonated form thereof one of R^{2a}, R^{2b}, R^{2c} and R^{2d} can be

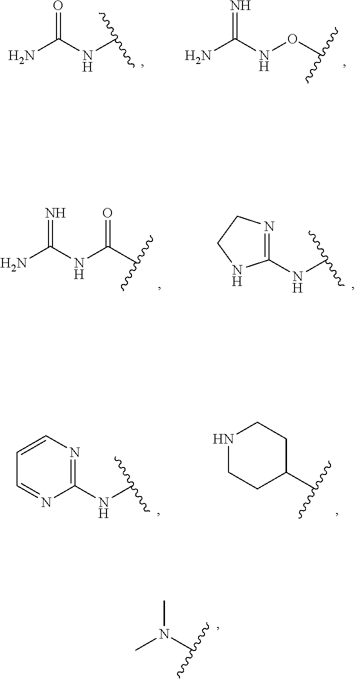

or a protonated form thereof. At least one of R^{2a}, R^{2b}, R^{2c} and R^{2d} can be

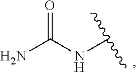 [SMILES: *NC(N)=O]

or a protonated form thereof, and the remaining of R^{2a}, R^{2b}, R^{2c} and R^{2d} can be guanidine or a protonated form thereof. At least two of R^{2a}, R^{2b}, R^{2c} and R^{2d} can be

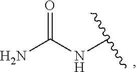 [SMILES: *NC(N)=O]

or a protonated form thereof, and the remaining of R^{2a}, R^{2b}, R^{2c} and R^{2d} can be guanidine, or a protonated form thereof.

All of R^{2a}, R^{2b}, R^{2c} and R^{2d} can be

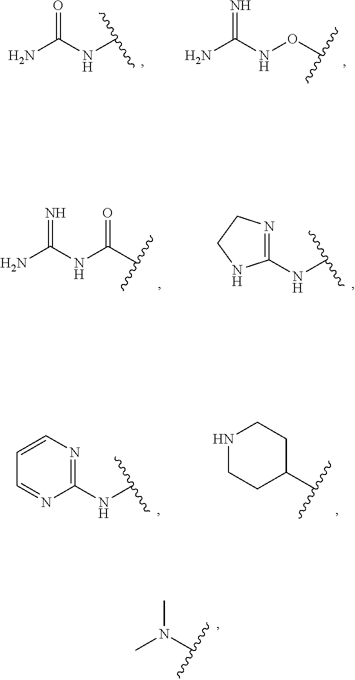

or a protonated form thereof. At least of R^{2a}, R^{2b}R^{2c} and R^{2d} can be

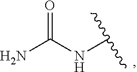 [SMILES: *NC(N)=O]

or a protonated form thereof, and the remaining of R^{2a}, R^{2b}, R^{2c} and R^{2d} can be guanidine or a protonated form thereof. At least two R^{2a}, R^{2b}, R^{2c} and R^{2d} groups can be

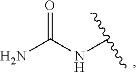 [SMILES: *NC(N)=O]

or a protonated form thereof, and the remaining of R^{2a}, R^{2b}, R^{2c} and R^{2d} are guanidine, or a protonated form thereof.

Each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

AA_{SC} can be

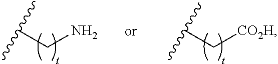

wherein t can be an integer from 0 to 5. AA_{SC} can be

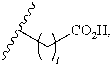

wherein t can be an integer from 0 to 5. t can be 1 to 5. t is 2 or 3. t can be 2. t can be 3.

R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

The cCPP of Formula (II) can have the structure of Formula (II-1):

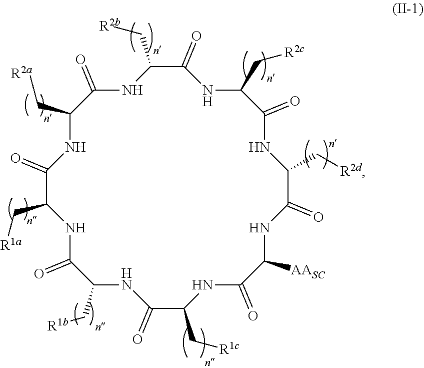

wherein R^{1a}, R^{1b}, R^{1c}, R^{2a}, R^{2b}, R^{2c}, R^{2d}, AA_{SC}, n′ and n″ are as defined herein.

The cCPP of Formula (II) can have the structure of Formula (IIa):

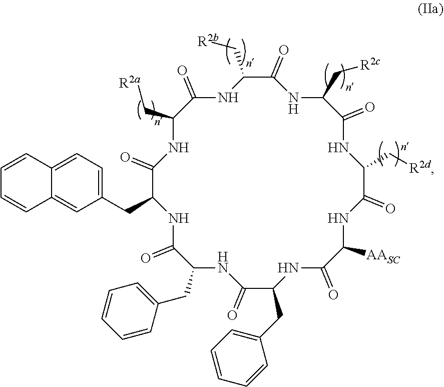

wherein R^{1a}, R^{1b}, R^{1c}, R^{2a}, R^{2b}, R^{2c}, R^{2d}, AA_{SC} and n′ are as defined herein.

The cCPP of formula (II) can have the structure of Formula (IIb):

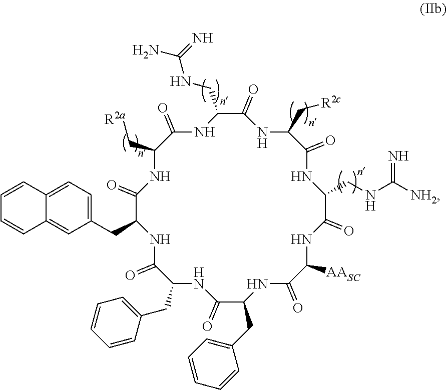

wherein R^{2a}, R^{2b}, AA_{SC}, and n′ are as defined herein.

The cCPP can have the structure of Formula (IIc):

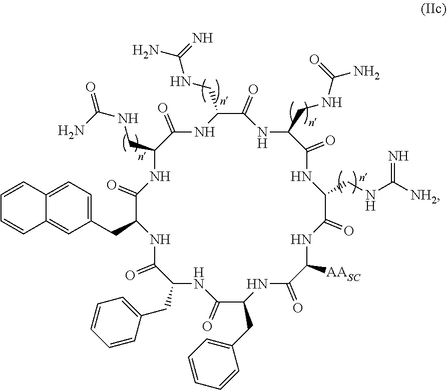

or a protonated form thereof,

wherein:

AA_{SC} and n′ are as defined herein.

The cCPP of Formula (IIa) has one of the following structures:

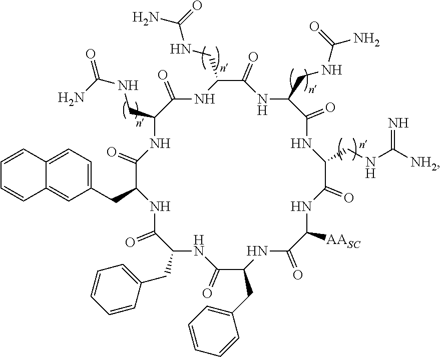

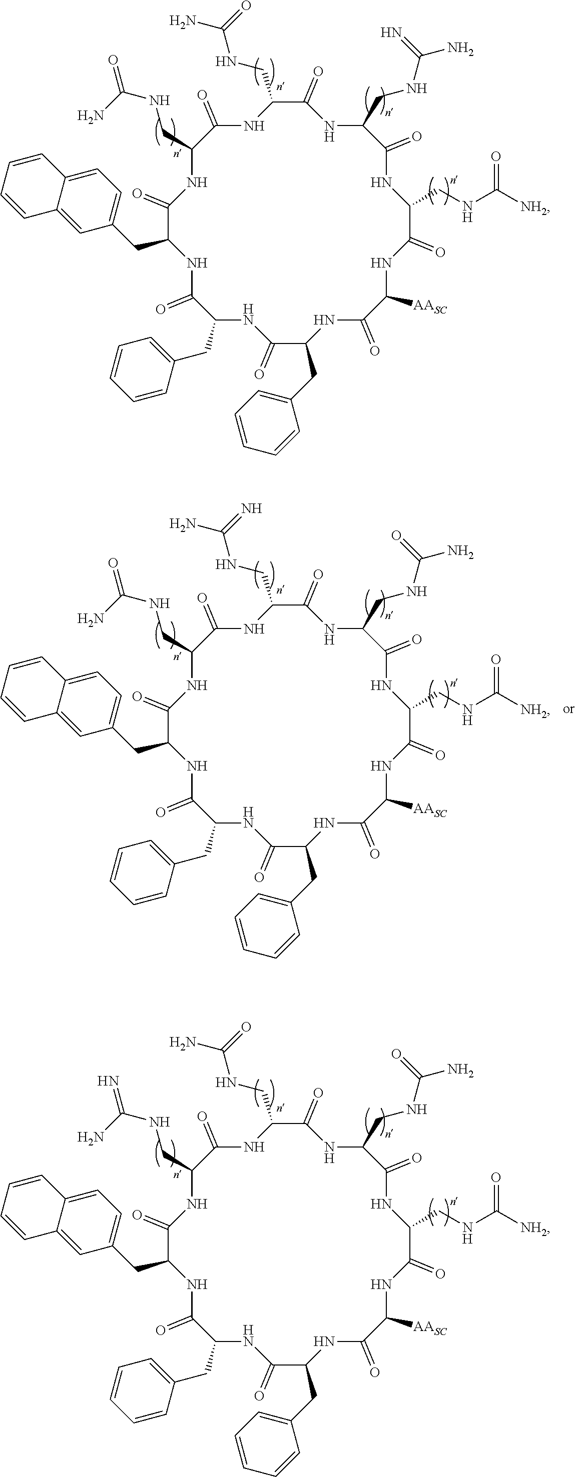

wherein AA_{SC} and n areas defined herein.

The cCPP of Formula (IIa) has one of the following structures:

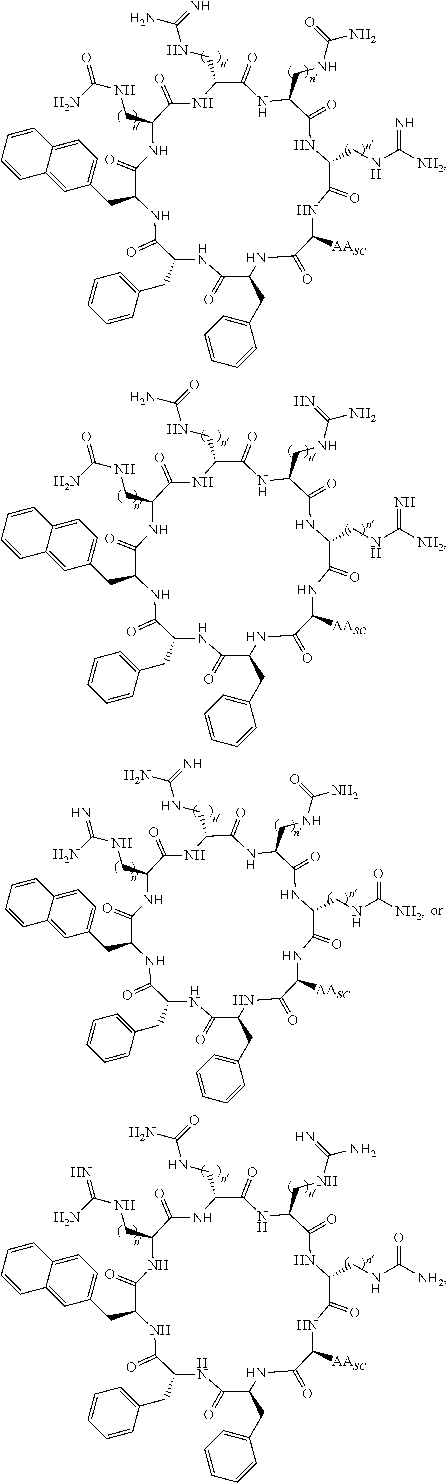

wherein AA_{SC} and n are as defined herein

The cCPP of Formula (IIa) has one of the following structures:

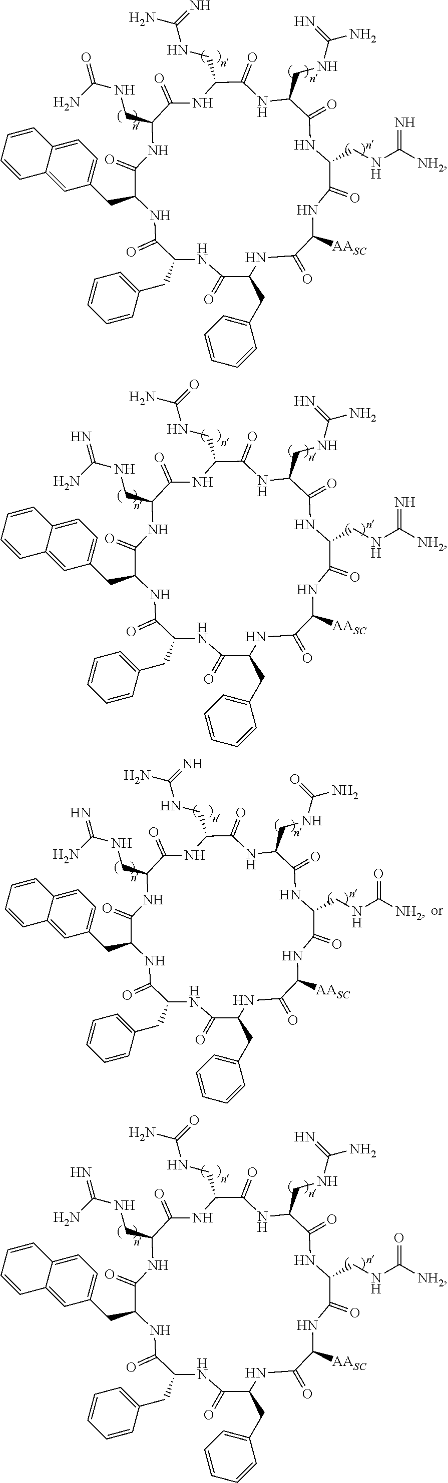

wherein AA_{SC} and n are as defined herein.

The cCPP of Formula (II) can have the structure.

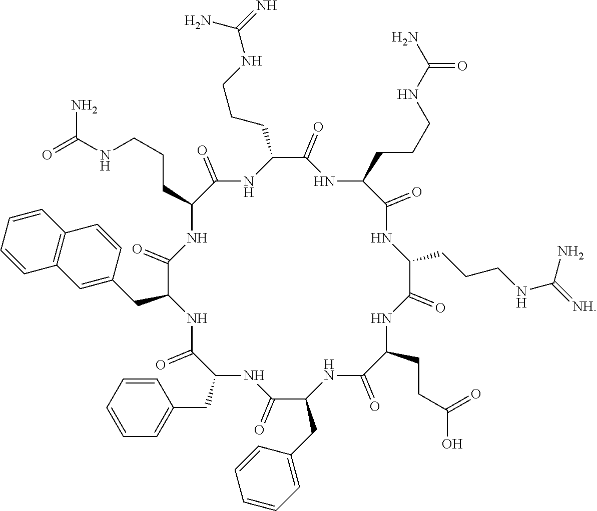 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (II) can have the structure:

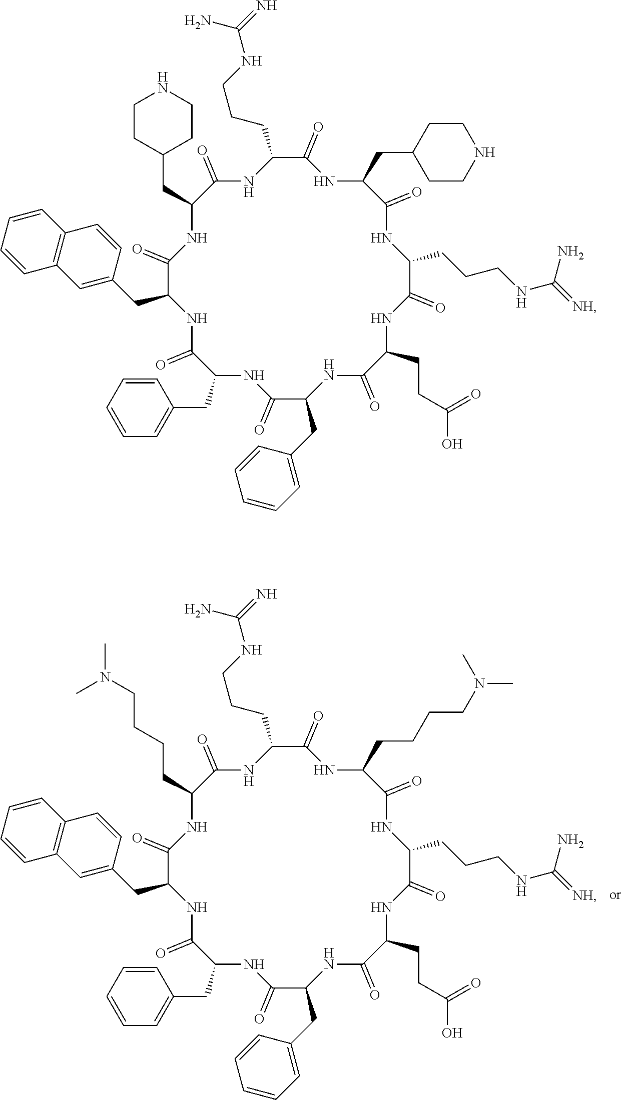 [SMILES: N=C(N)NCCC[C@H]1NC(=O)[C@H](CC2CCNCC2)NC(=O)[C@@H](CCCNC(=N)N)NC(=O)[C@H](CC2CCNCC2)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O ; CN(C)CCCC[C@@H]1NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)[C@H](CCCCN(C)C)NC(=O)[C@@H](CCCNC(=N)N)NC1=O]

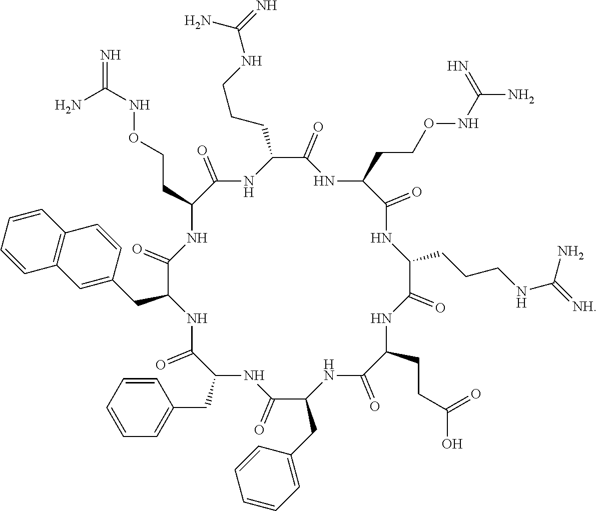 [SMILES: N=C(N)NCCC[C@H]1NC(=O)[C@H](CCONC(=N)N)NC(=O)[C@@H](CCCNC(=N)N)NC(=O)[C@H](CCONC(=N)N)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP can have the structure of Formula (III):

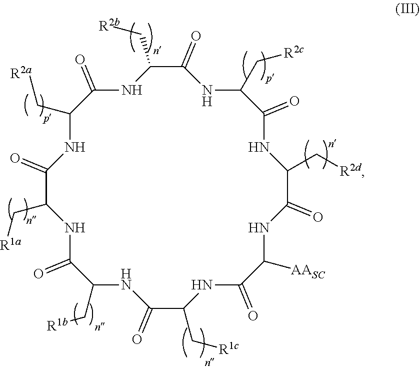

wherein:

AA_{SC} is an amino acid side chain;

R^{1a}, R^{1b}, and R^{1c} are each independently a 6- to 14-membered aryl or a 6- to 14-membered heteroaryl;

R^{2a} and R^{2c} are each independently

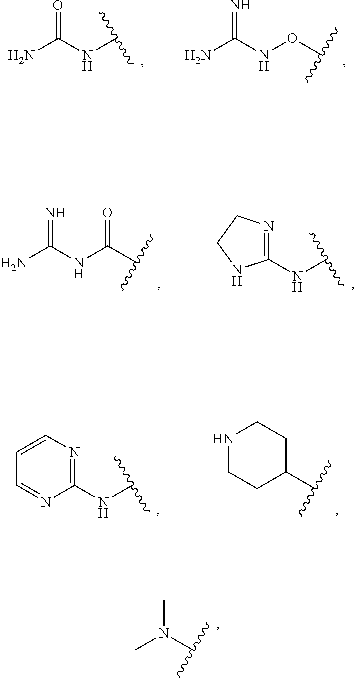

or a protonated form thereof,

R^{2b} and R^{2d} are each independently guanidine or a protonated form thereof;

each n″ is independently an integer from 1 to 3;

each n′ is independently an integer from 1 to 5; and

each p′ is independently an integer from 0 to 5.

The cCPP of Formula (III) can have the structure of Formula (III-1):

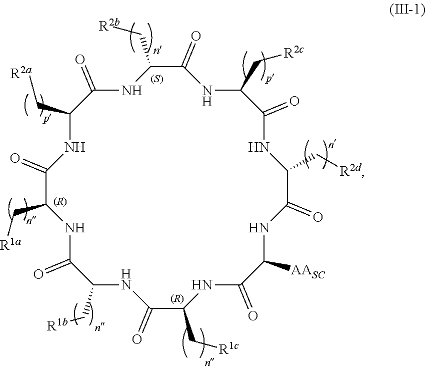

wherein:

AA_{SC}, R^{1a}, R^{1b}, R^{1c}, R^{2a}, R^{2c}, R^{2b}, R^{2d} n′, n″, and p′ are as defined herein.

The cCPP of Formula (III) can have the structure of Formula (IIIa):

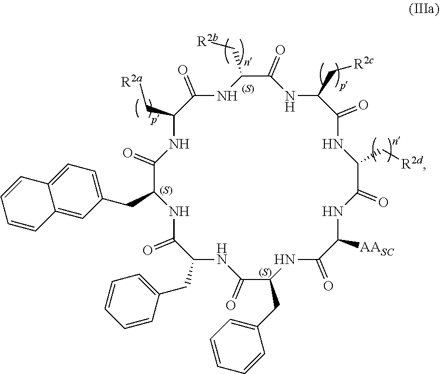

wherein:

AA_{SC}, R^{2a}, R^{2c}, R^{2b}, R^{2d} n′, n″, and p′ are as defined herein.

In Formulas (III), (III-1), and (IIIa), Rₐ and R can be H. R^{a} and R can be H and R^{b} and R^{d} can each independently be guanidine or protonated form thereof. R^{a} can be H. R^{b} can be H. p′ can be 0. R^{a} and R can be H and each p′ can be 0.

In Formulas (III), (III-1), and (IIIa), R^{a} and R can be H, R^{b} and R^{d} can each independently be guanidine or protonated form thereof, n″ can be 2 or 3, and each p′ can be 0.

p′ can 0. p′ can 1. p′ can 2. p′ can 3. p′ can 4. p′ can be 5.

The cCPP can have the structure:

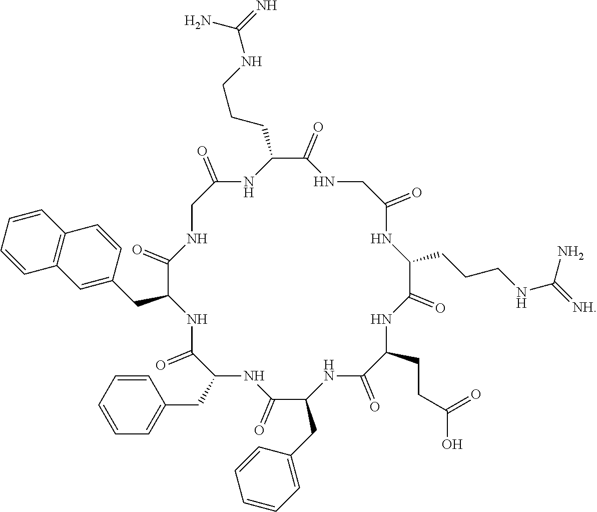 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

The cCPP of Formula (A) can be selected from:

| CPP Sequence | SEQ ID NO: |
|---|---|
| (FfΦRrRrQ) | |
| (FfΦCit-r-Cit-rQ) | |
| (FfΦGrGrQ) | |
| (FfFGRGRQ) | |
| (FGFGRGRQ) | 298 |
| (GfFGrGrQ) | |
| (FGFGRRRQ) | 299 |
| (FGFRRRRQ) | 300 |

The cCPP of Formula (A) can be selected from:

| CPP Sequence | SEQ ID NO: |
|---|---|
| FΦPRRRRQ | 301 |
| fΦRrRrQ | |
| FfΦRrRrQ | |
| FfΦCit-r-Cit-rQ | |
| FfΦGrGrQ | |
| FfΦRGRGQ | |
| FfFGRGRQ | |
| FGFGRGRQ | 298 |
| GfFGrGrQ | |
| FGFGRRRQ | 299 |
| FGFRRRRQ | 300 |

In embodiments, the cCPP is selected from:

CPP sequences

| | | | | | |
|---|---|---|---|---|---|
| FΦRRRQ | 302 | RRFRΦRQ | 308 | FΦRRRRQK | 315 |
| FΦRRRC | 303 | FRRRRΦQ | 309 | FΦRRRRQC | 316 |
| FΦRRRU | 304 | rRFRΦRQ | | fΦRrRrRQ | |
| RRRΦFQ | 305 | RRΦFRRQ | 310 | FΦRRRRRQ | 317 |
| RRRRΦF | 306 | CRRRRFWQ | 311 | RRRRΦFDΩC | 318 |
| FΦRRRR | 307 | FfΦRrRrQ | | FΦRRR | 319 |
| FϕrRrRq | | FFΦRRRRQ | 312 | FWRRR | 320 |
| FϕrRrRQ | | RFRFRΦRQ | 313 | RRRΦF | 321 |
| FΦRRRRQ | 301 | URRRRFWQ | 314 | RRRWF | 322 |
| fΦRrRrQ | | CRRRRFWQ | 311 | | |

Where Φ = L-naphthylalanine;
ϕ= D-naphthylalanine;
Ω = L-norleucine

In embodiments, the cCPP is not selected from:

CPP sequences

| | | | | | |
|---|---|---|---|---|---|
| FΦRRRQ | 302 | RRFRΦRQ | 308 | FΦRRRRQK | 315 |
| FΦRRRC | 303 | FRRRRΦQ | 309 | FΦRRRRQC | 316 |
| FΦRRRU | 304 | rRFRΦRQ | | fΦRrRrRQ | |
| RRRΦFQ | 305 | RRΦFRRQ | 310 | FΦRRRRRQ | 317 |
| RRRRΦF | 306 | CRRRRFWQ | 311 | RRRRΦFDΩC | 318 |
| FΦRRRR | 307 | FfΦRrRrQ | | FΦRRR | 319 |
| FϕrRrRq | | FFΦRRRRQ | 312 | FWRRR | 320 |
| FϕrRrRQ | | RFRFRΦRQ | 313 | RRRΦF | 321 |
| FΦRRRRQ | 301 | URRRRFWQ | 314 | RRRWF | 322 |
| fΦRrRrQ | | CRRRRFWQ | 311 | | |

Where Φ = L-naphthylalanine;
ϕ = D-naphthylalanine;
Ω = L-norleucine

AA_{SC} can be conjugated to a linker.

Linker

The cCPP of the disclosure can be conjugated to a linker. The linker can link a cargo to the cCPP. The linker can be attached to the side chain of an amino acid of the cCPP, and the cargo can be attached at a suitable position on linker.

The linker can be any appropriate moiety which can conjugate a cCPP to one or more additional moieties, e.g., an exocyclic peptide (EP) and/or a cargo. Prior to conjugation to the cCPP and one or more additional moieties, the linker has two or more functional groups, each of which are independently capable of forming a covalent bond to the cCPP and one or more additional moieties. If the cargo is an oligonucleotide, the linker can be covalently bound to the 5′ end of the cargo or the 3′ end of the cargo. The linker can be covalently bound to the 5′ end of the cargo. The linker can be covalently bound to the 3′ end of the cargo. If the cargo is a peptide, the linker can be covalently bound to the N-terminus or the C-terminus of the cargo. The linker can be covalently bound to the backbone of the oligonucleotide or peptide cargo. The linker can be any appropriate moiety which conjugates a cCPP described herein to a cargo such as an oligonucleotide, peptide or small molecule.

The linker can comprise hydrocarbon linker.

The linker can comprise a cleavage site. The cleavage site can be a disulfide, or caspase-cleavage site (e.g, Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹-J-R²)z″- subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) —(R¹-J)z″- or -(J-R¹)z″-, wherein each of R¹, at each instance, is independently alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or —(R¹-J-R²)z″-, wherein each of R¹ and R², at each instance, are independently alkylene, each J is independently C, NR³, —NR³C(O)—, S, and 0, wherein R⁴ is independently selected from H and alkyl, and z″ is an integer from 1 to 50; or combinations thereof.

The linker can comprise a —(OCH₂CH₂)_{z′}— (e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂)z′ can also be referred to as polyethylene glycol (PEG).

The linker can comprise one or more amino acids. The linker can comprise a peptide. The linker can comprise a —(OCH₂CH₂)_{z′}—, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the cargo is conjugated to the linker.

The linker can comprises (i) a β alanine residue and lysine residue; (ii) -(J-R¹)z″; or (iii) a combination thereof. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of β-alanine, glycine, lysine, 4-aminobutyric acid, 5-aminopentanoic acid, 6-aminohexanoic acid or combinations thereof; and (ii) —(R¹-J)z″- or -(J-R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O. The linker can comprise glycine, beta-alanine, 4-aminobutyric acid, 5-aminopentanoic acid, 6-aminohexanoic acid, or a combination thereof.

The linker can be a trivalent linker. The linker can have the structure:

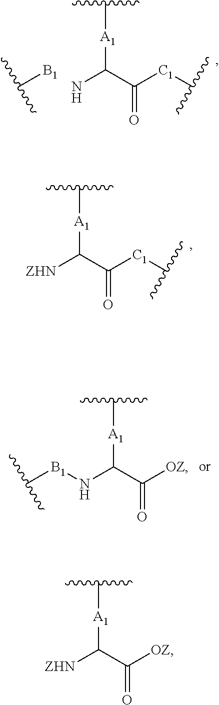

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ₙ—COOH], or caspase-cleavage site (Val-Cit-PABC).

The hydrocarbon can be a residue of glycine or beta-alanine.

The linker can be bivalent and link the cCPP to a cargo. The linker can be bivalent and link the cCPP to an exocyclic peptide (EP).

The linker can be trivalent and link the cCPP to a cargo and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The linker can have the structure:

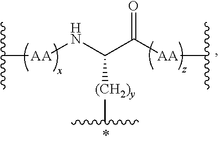

wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

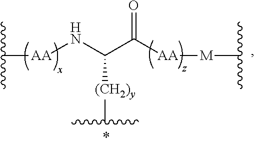

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

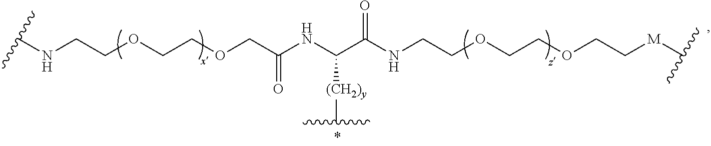

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

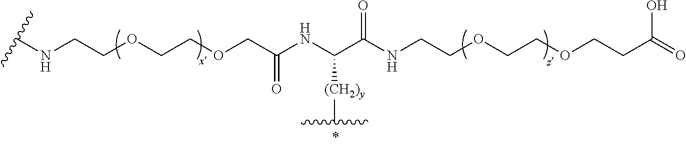

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

x can be an integer from 1-10, e.g., 1, 2, 3, 4, 5, 6, 7, 8, 9, or 10, inclusive of all ranges and subranges therebetween.

x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.

y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.

z can be an integer from 1-10, e.g., 1, 2, 3, 4, 5, 6, 7, 8, 9, or 10, inclusive of all ranges and subranges therebetween.

z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is part of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can be bound to the side chain of aspartic acid, glutamic acid, glutamine, asparagine, or lysine, or a modified side chain of glutamine or asparagine (e.g., a reduced side chain having an amino group), on a peptide cargo. The linker can be bound to the side chain of lysine on the peptide cargo.

The linker can have a structure:

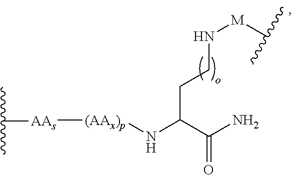

wherein

M is a group that conjugates L to a cargo, for example, an oligonucleotide;

AAₛ is a side chain or terminus of an amino acid on the cCPP;

each AAₓ is independently an amino acid residue;

o is an integer from 0 to 10; and

p is an integer from 0 to 5.

The linker can have a structure:

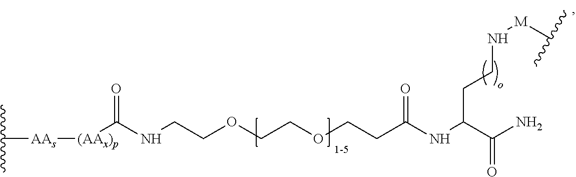

wherein

M is a group that conjugates L to a cargo, for example, an oligonucleotide; AAₛ is a side chain or terminus of an amino acid on the cCPP;

each AAₓ is independently an amino acid residue;

o is an integer from 0 to 10; and

p is an integer from 0 to 5.

M can comprise an alkylene, alkenylene, alkynylene, carbocyclyl, or heterocyclyl, each of which is optionally substituted. M can be selected from:

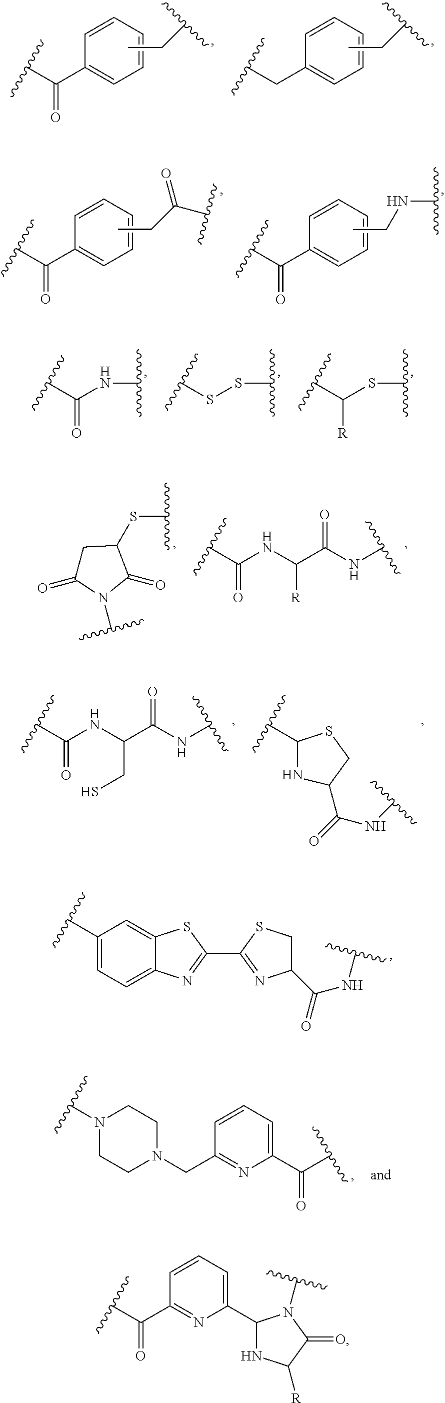

wherein R is alkyl, alkenyl, alkynyl, carbocyclyl, or heterocyclyl.

M can be selected from:

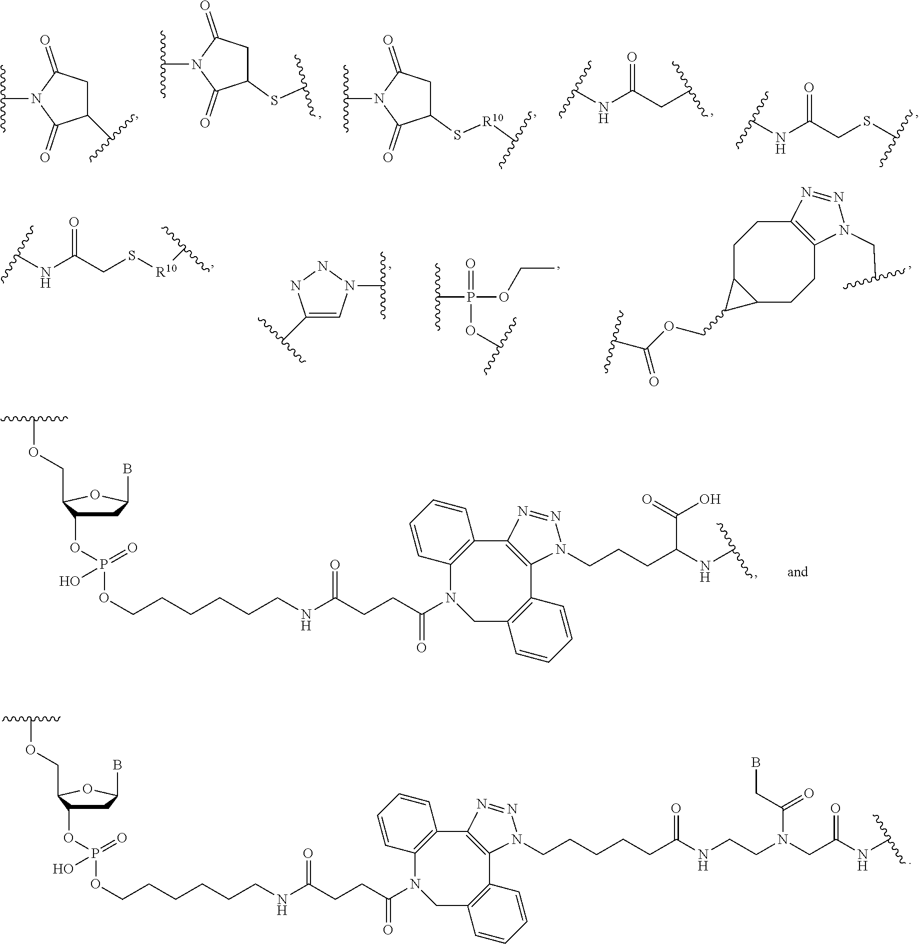

wherein: R¹⁰ is alkylene, cycloalkyl, or

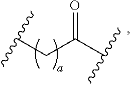

M can be

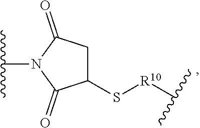

### R¹⁰ can be

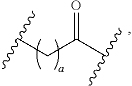

### and a is 0 to 10. M can be.

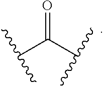 [SMILES: *C(*)=O]

M can be a heterobifunctional crosslinker, e.g.,

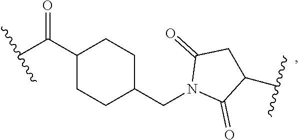 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The β-amino acid can be β-alanine.

o can be an integer from 0 to 10, e.g., 0, 1, 2, 3, 4, 5, 6, 7, 8, 9, and 10. o can be 0, 1, 2, or 3. o can be 0. o can be 1. o can be 2. o can be 3.

p can be 0 to 5, e.g., 0, 1, 2, 3, 4, or 5. p can be 0. p can be 1. p can be 2. p can be 3. p can be 4. p can be 5.

The linker can have the structure:

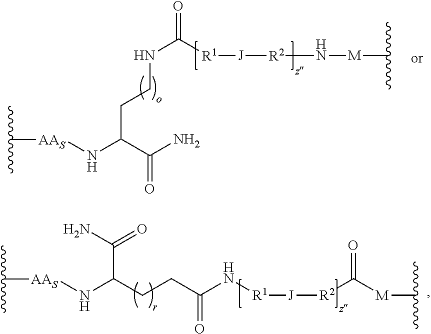

wherein M, AAₛ, each —(R¹-J-R²)z″-, o and z″ are defined herein; r can be 0 or 1.

r can be 0. r can be 1.

The linker can have the structure:

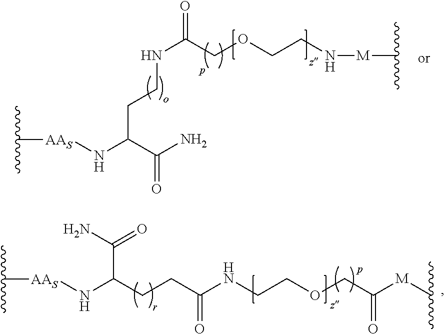

wherein each of M, AAₛ, o, p, q, r and z″ can be as defined herein.

z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

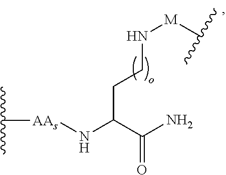

wherein:

M, AAₛ and o are as defined herein.

Other non-limiting examples of suitable linkers include:

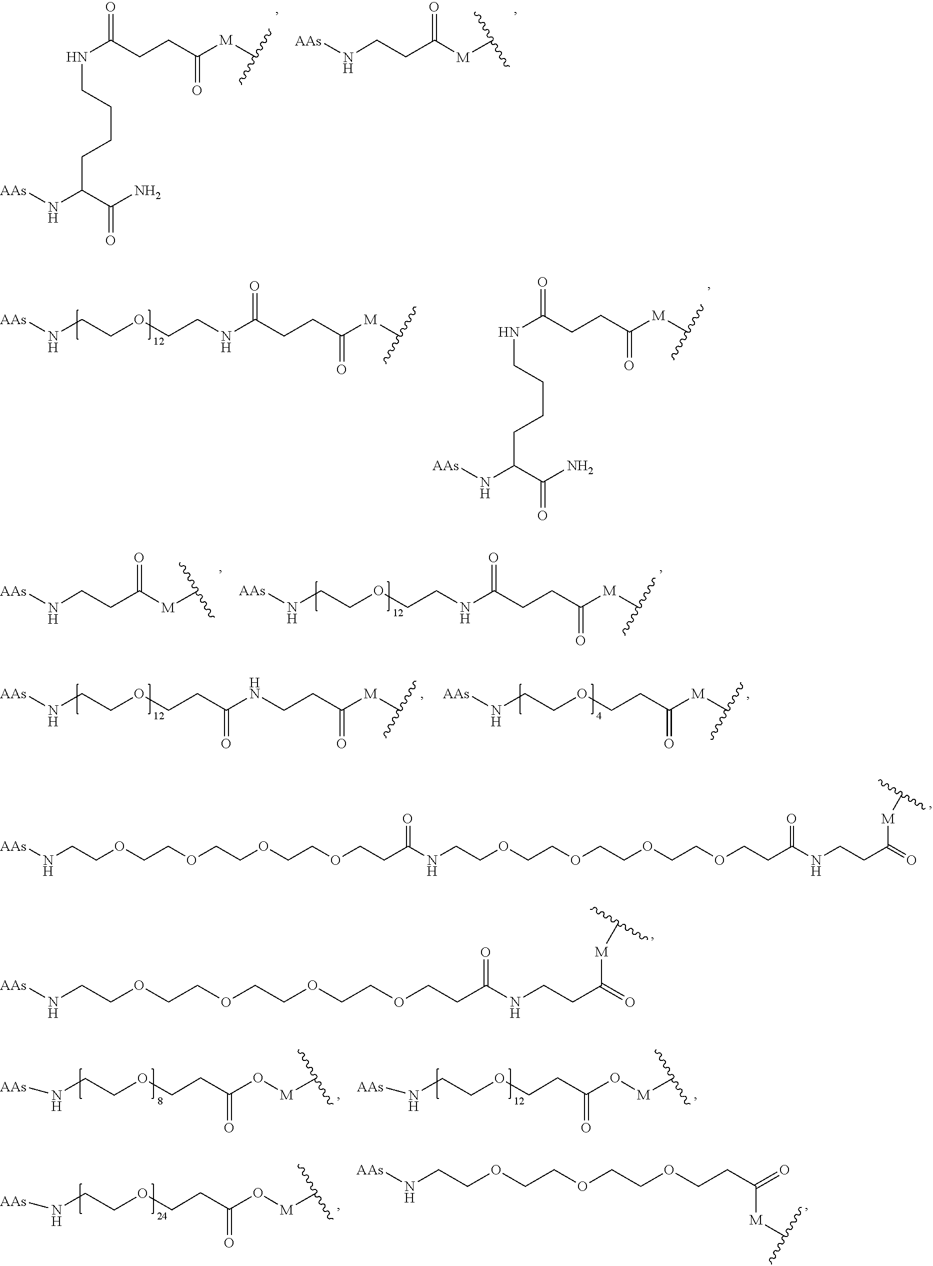

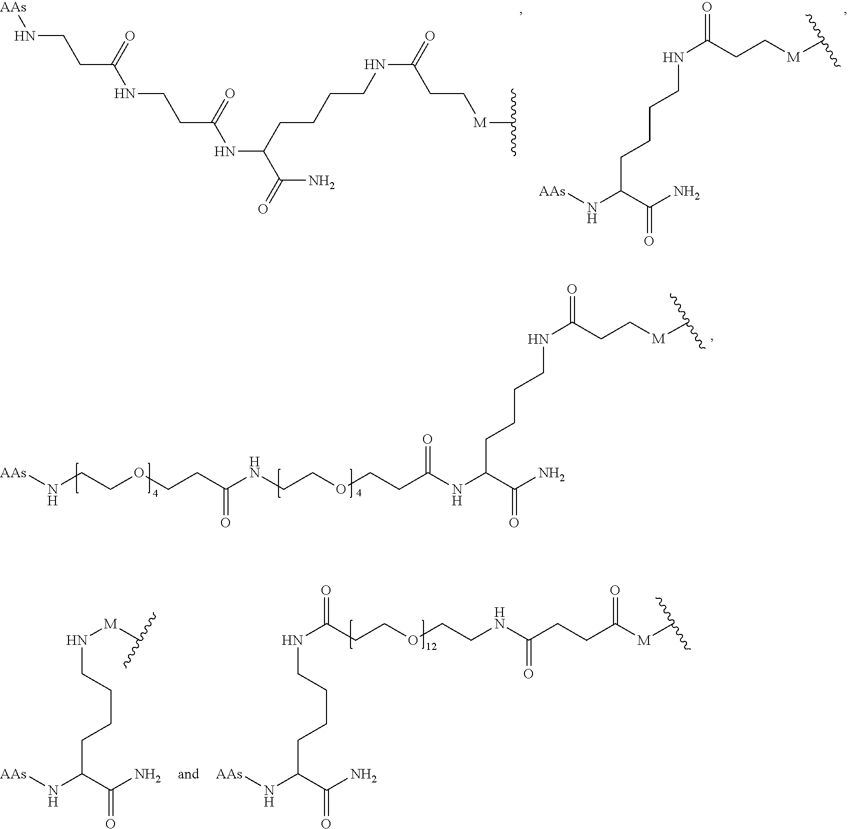

wherein M and AAₛ are as defined herein.

Provided herein is a compound comprising a cCPP and an AC that is complementary to a target in a pre-mRNA sequence further comprising L, wherein the linker is conjugated to the AC through a bonding group (M), wherein M is O

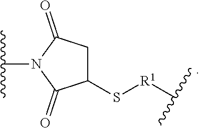

Provided herein is a compound comprising a cCPP and a cargo that comprises an antisense compound (AC), for example, an antisense oligonucleotide, that is complementary to a target in a pre-mRNA sequence, wherein the compound further comprises L, wherein the linker is conjugated to the AC through a bonding group (M), wherein M is selected from:

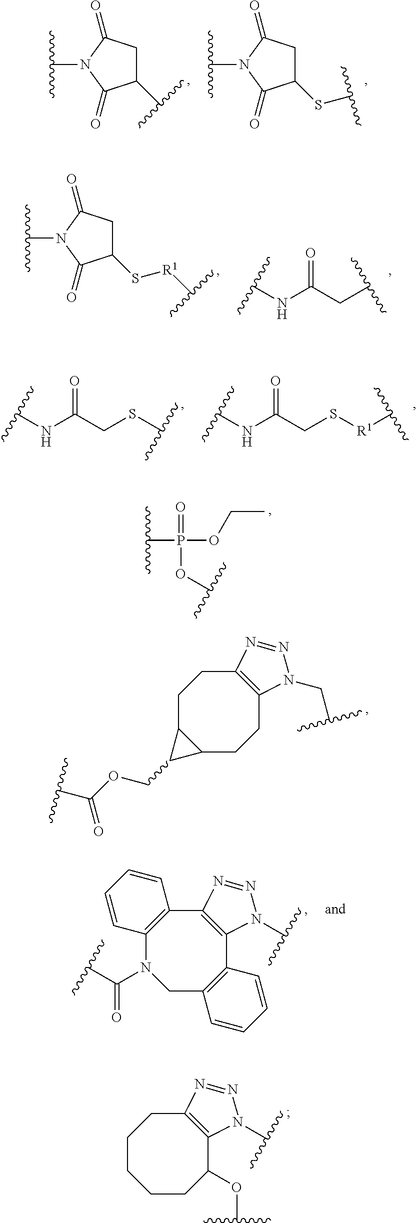

wherein: R¹ is alkylene, cycloalkyl, or

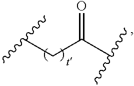

wherein t′ is 0 to 10 wherein each R is independently an alkyl, alkenyl, alkynyl, carbocyclyl, or heterocyclyl, wherein R¹ is

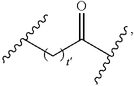

and t′ is 2.

The linker can have the structure:

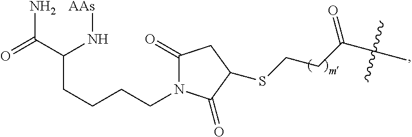

wherein AAₛ is as defined herein, and m′ is 0-10.

The linker can be of the formula:

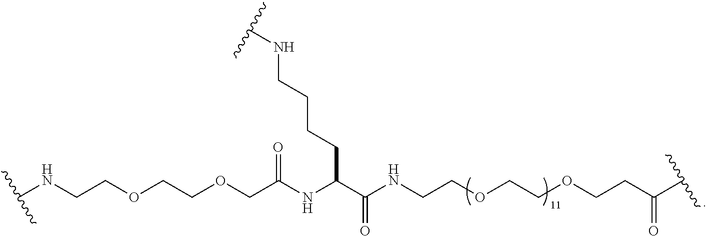

The linker can be covalently bound to a cargo at any suitable location on the cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

cCPP-Linker Conjugates

The cCPP can be conjugated to a linker defined herein. The linker can be conjugated to an AA_{SC} of the cCPP as defined herein.

The linker can comprise a —(OCH₂CH₂)_{z′}— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z′} is also referred to as PEG. The cCPP-linker conjugate can have a structure selected from Table 16:

TABLE 16
cCPP-linker conjugates
cyclo(FfΦ-4gp-r-4gp-rQ)-PEG₄-K-NH₂
cyclo(FfΦ-Cit-r-Cit-rQ)-PEG₄-K-NH₂
cyclo(FfΦ-Pia-r-Pia-rQ)-PEG₄-K-NH₂
cyclo(FfΦ-Dml-r-Dml -rQ)-PEG₄-K-NH₂
cyclo(FfΦ-Cit-r-Cit-rQ)-PEG₁₂-OH
cyclo(fΦR-Cit-R-Cit-Q)-PEG₁₂-OH
cyclo(fΦ-Can-r-Can-r-Q)-PEG₁₂-OH
cyclo(FfΦ-Can-r-Can-r-Q)-PEG₁₂-OH

The linker can comprise a —(OCH₂CH₂)_{z′}— subunit, wherein z′ is an integer from 1 to 23, and a peptide subunit. The peptide subunit can comprise from 2 to 10 amino acids. The cCPP-linker conjugate can have a structure selected from Table 17:

TABLE 17
cCPP-linker conjugate
Ac-PKKK-Can-KV-PEG₂-K(cyclo(FfΦ-Can-r-Can-r-Q)-PEG₁₂-K(N₃)-NH₂
Ac-PKKKRKV-Lys(cyclo[FfΦ-R-r-Cit-rQ])-PEG₁₂-K(N₃)-NH₂
Ac-PKKKRKV-Lys(cyclo[FfΦ-Cit-r-R-rQ])-PEG₁₂-K(N₃)-NH₂
Ac-PKKKRKV-K(cyclo(FfΦR-cit-R-cit-Q))-PEG₁₂-K(N₃)-NH₂
Ac-PKKKRKV-PEG2-Lys(cyclo[FfΦ-Cit-r-Cit-rQ])-B-k(N₃)-NH₂
Ac-PKKKRKV-PEG2-Lys(cyclo[FfΦ-Cit-r-Cit-rQ])-PEG2-k(N₃)-NH₂
Ac-PKKKRKV-PEG2-Lys(cyclo[FfΦ-Cit-r-Cit-rQ])-PEG4-k(N₃)-NH₂
Ac-PKKKRKV-Lys(cyclo[FfΦ-Cit-r-Cit-rQ])-PEG12-k(N₃)-NH₂
Ac-pkkkrkv-PEG2-Lys(cyclo[FfΦ-Cit-r-Cit-rQ])-PEG12-k(N₃)-NH₂
Ac-Trv-PEG2-Lys(cyclo[FfΦ-Cit-r-Cit-rQ])-PEG12-OH
Ac-PKKKRKV-PEG2-Lys(cyclo[FfΦ-Cit-r-Cit-r-Q])-PEG12-k(N₃)-NH₂
Ac-PKKK-Cit-KV-PEG2-Lys(cyclo[FfΦ-Cit-r-Cit-r-Q])-PEG12-k(N₃)-NH₂
Ac-PKKKRKV-PEG2-Lys(cyclo[FfΦ-Cit-r-Cit-r-Q]-PEG12-K(N₃)-NH₂

EEVs comprising a cyclic cell penetrating peptide (cCPP), linker and exocyclic peptide (EP) are provided. An EEV can comprise the structure of Formula (B):

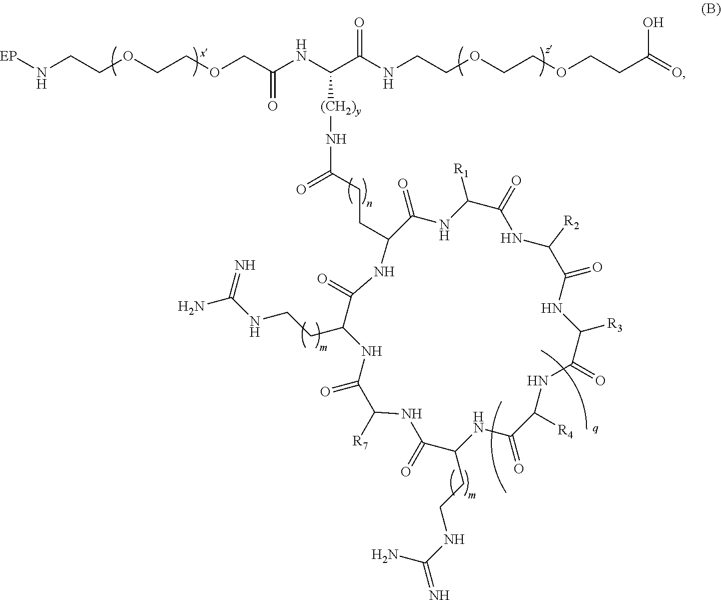

or a protonated form thereof,

wherein:

R₁, R₂, and R₃ are each independently H or an aromatic or heteroaromatic side chain of an amino acid;

R₄ and R₇ are independently H or an amino acid side chain;

EP is an exocyclic peptide as defined herein;

each m is independently an integer from 0-3;

n is an integer from 0-2;

x′ is an integer from 1-20;

y is an integer from 1-5;

q is 1-4; and

z′ is an integer from 1-23.

R₁, R₂, R₃, R₄, R₇, EP, m, q, y, x′, z′ are as described herein.

n can be 0. n can be 1. n can be 2.

The EEV can comprise the structure of Formula (B-a) or (B-b):

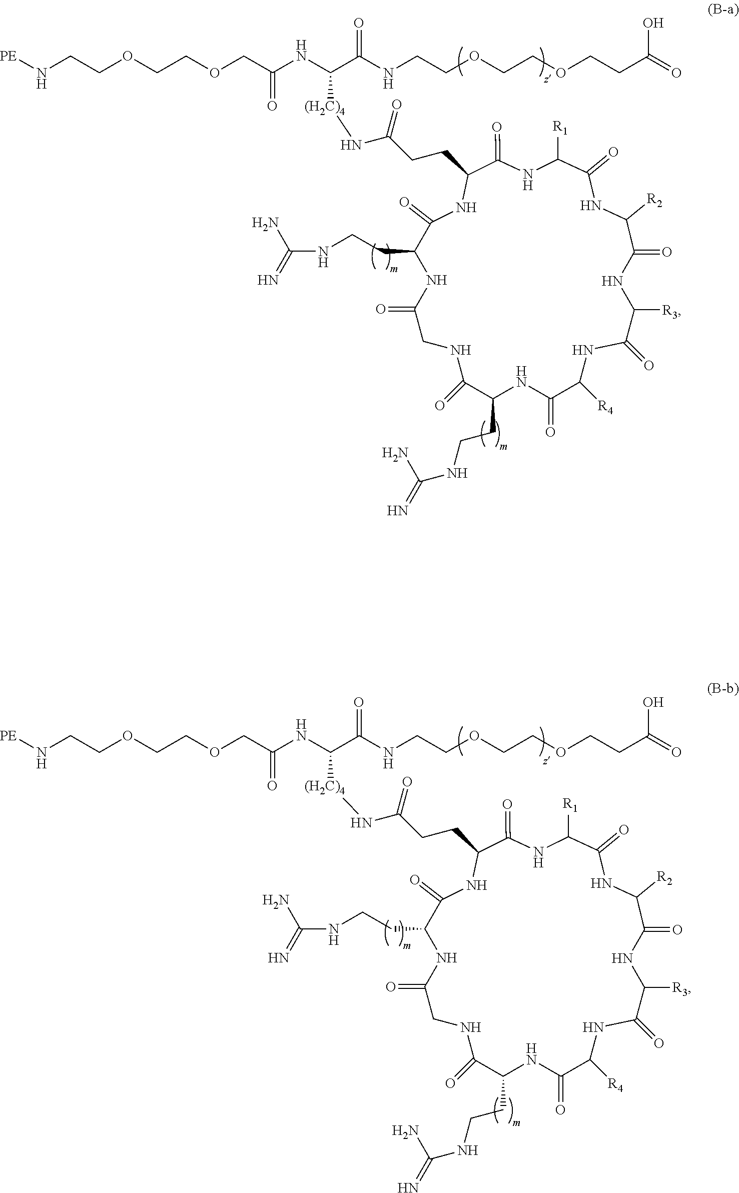

or a protonated form thereof, wherein EP (shown as “PE”), R¹, R², R³, R⁴, m and z′ are as defined above in Formula (B).

The EEV can comprises the structure of Formula (B-c):

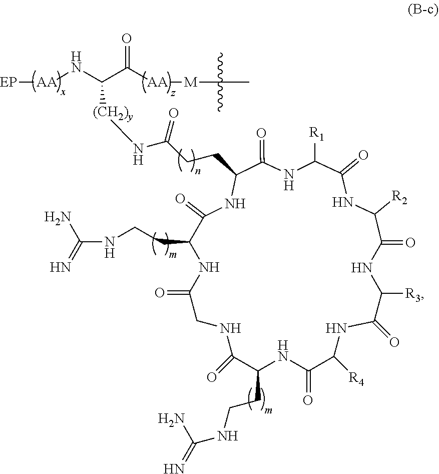

or a protonated form thereof, wherein EP, R¹, R², R³, R⁴, and m are as defined above in Formula (B); AA is an amino acid as defined herein; M is as defined herein; n is an integer from 0-2; x is an integer from 1-10; y is an integer from 1-5; and z is an integer from 1-10.

The EEV can have the structure of Formula (B-1), (B-2), (B-3), or (B-4):

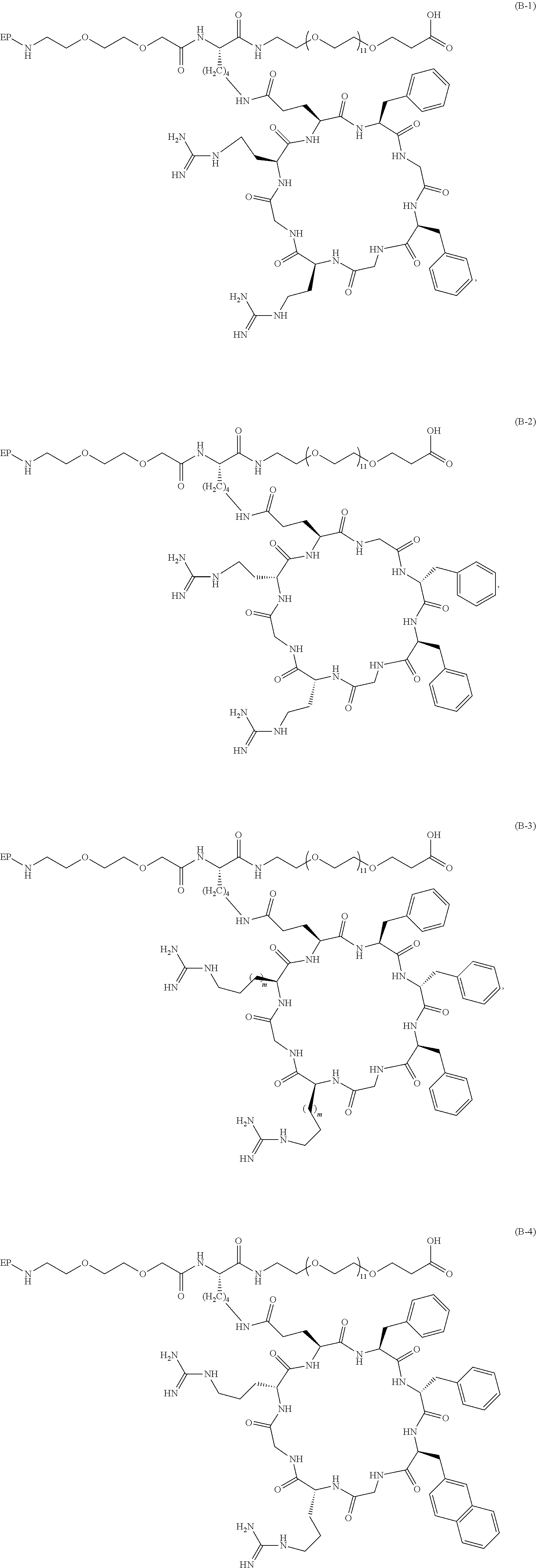

or a protonated form thereof, wherein EP is as defined above in Formula (B).

The EEV can comprise Formula (B) and can have the structure: Ac-PKKKRKVAEEA-K(cyclo[FGFGRGRQ])-PEG₁₂-OH or Ac-PK-KKR-KV-AEEA-K(cyclo[GfFGrGrQ])-PEG₁₂-OH.

The EEV can comprise a cCPP of formula:

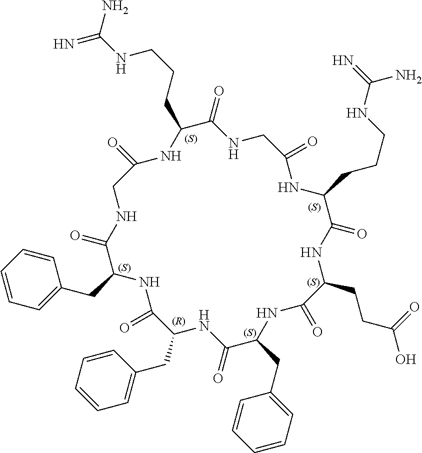

The EEV can comprise formula: Ac-PKKKRKV-miniPEG₂-Lys(cyclo(FfFGRGRQ)-PEG₂-K(N₃)).

The EEV can be:

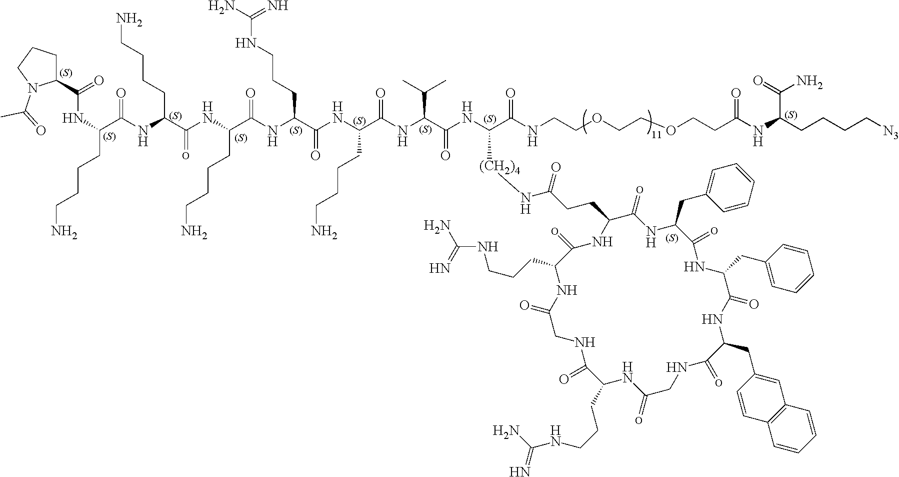

The EEV can be

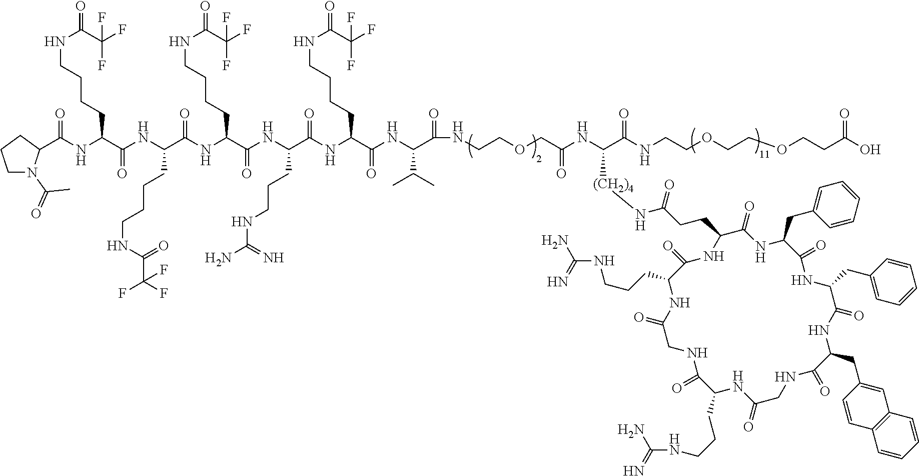

The EEV can be Ac-P-K(Tfa)-K(Tfa)-K(Tfa)-R-K(Tfa)-V-miniPEG₂-K(cyclo(Ff-Nal-GrGrQ)-PEG₁₂-OH.

The EEV can be

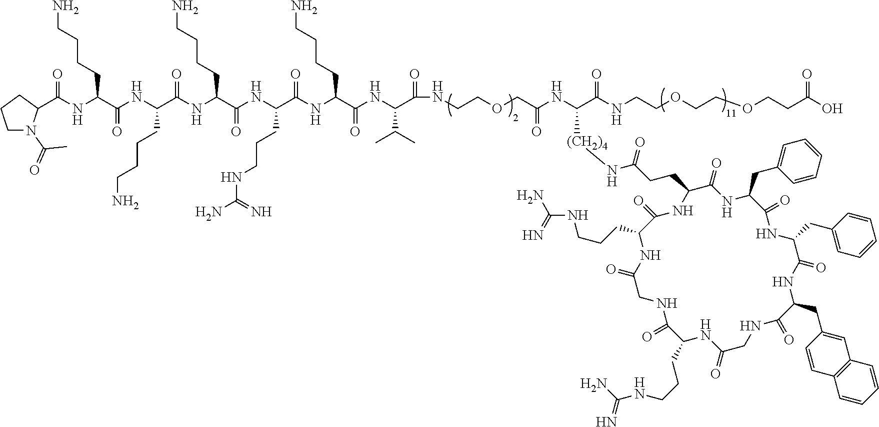

The EEV can be Ac-P-K-K-K-R-K-V-miniPEG₂-K(cyclo(Ff-Nal-GrGrQ)-PEG₁₂-OH.

The EEV can be

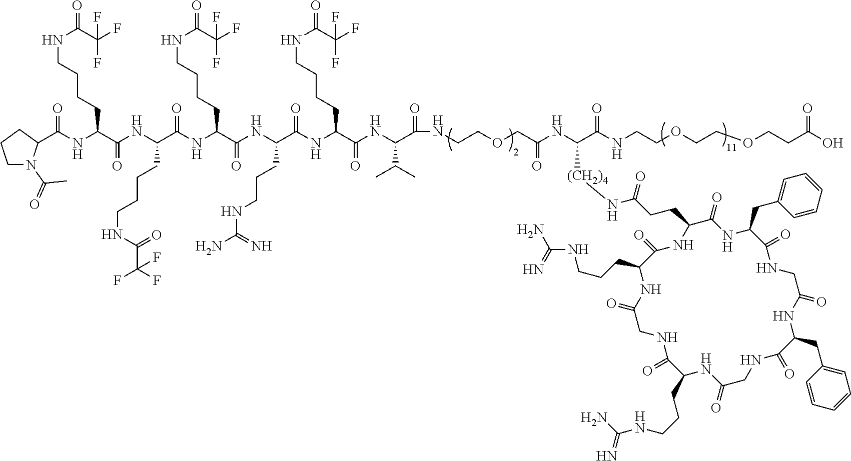

The EEV can be

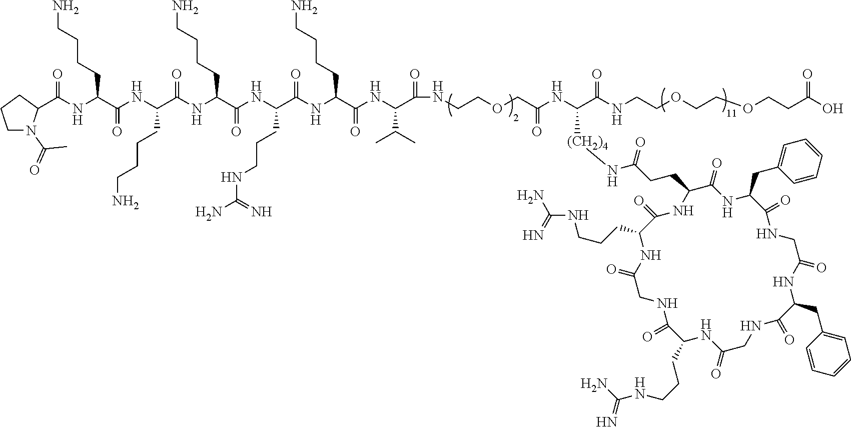

The EEV can be

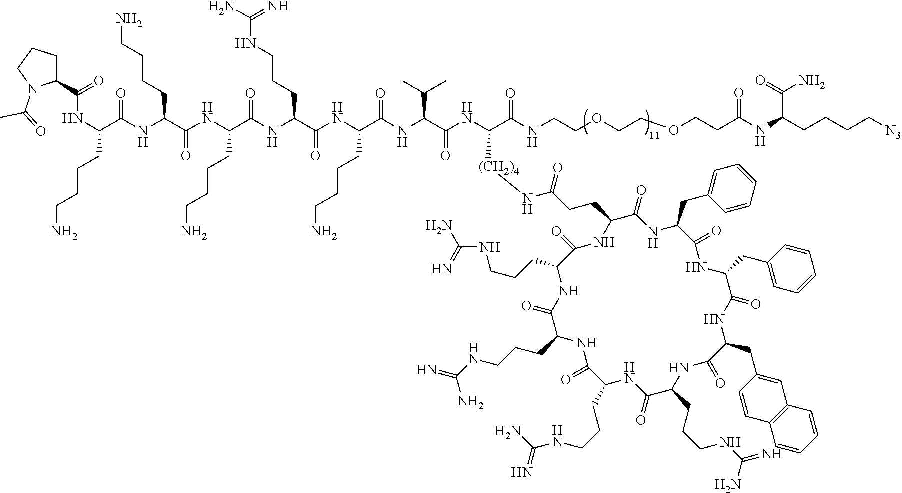

The EEV can be

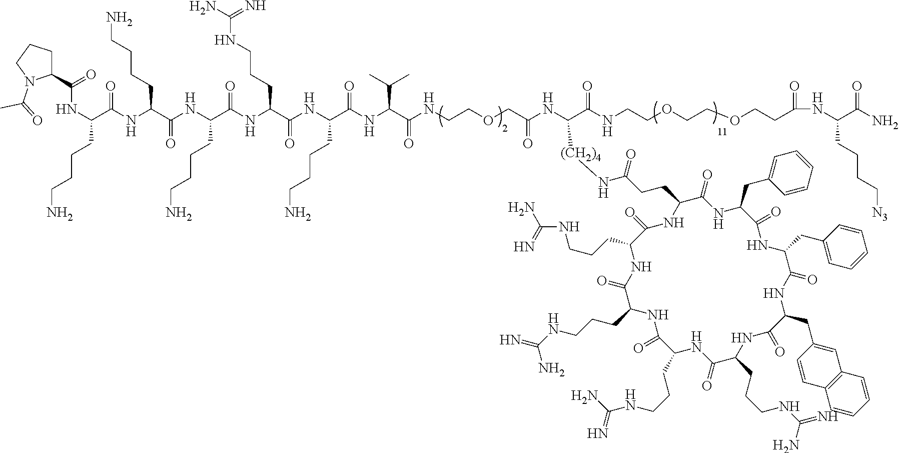

The EEV can be

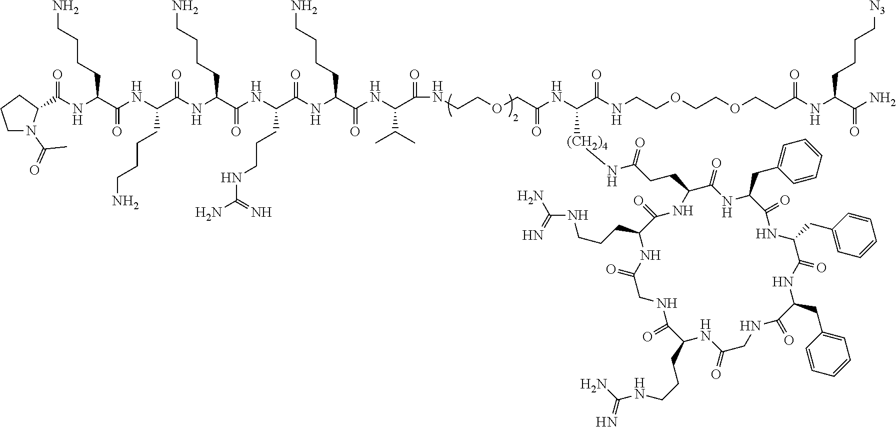

The EEV can be

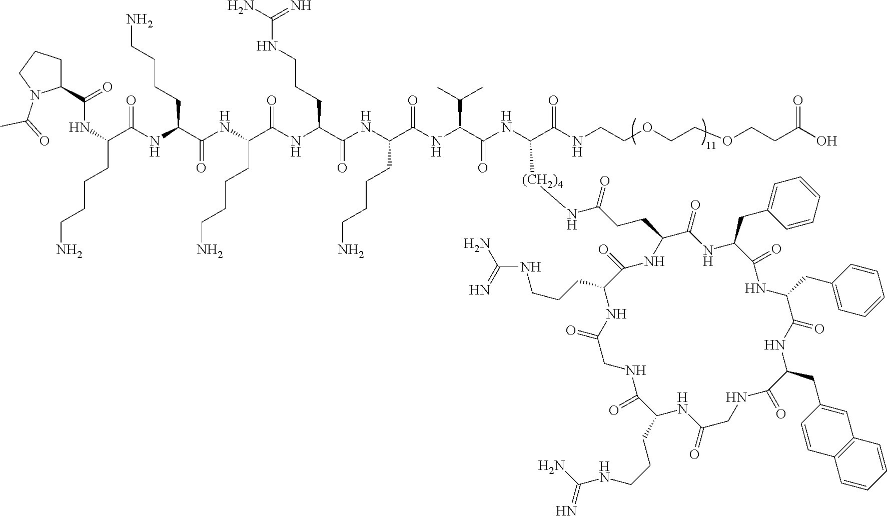

The EEV can be:

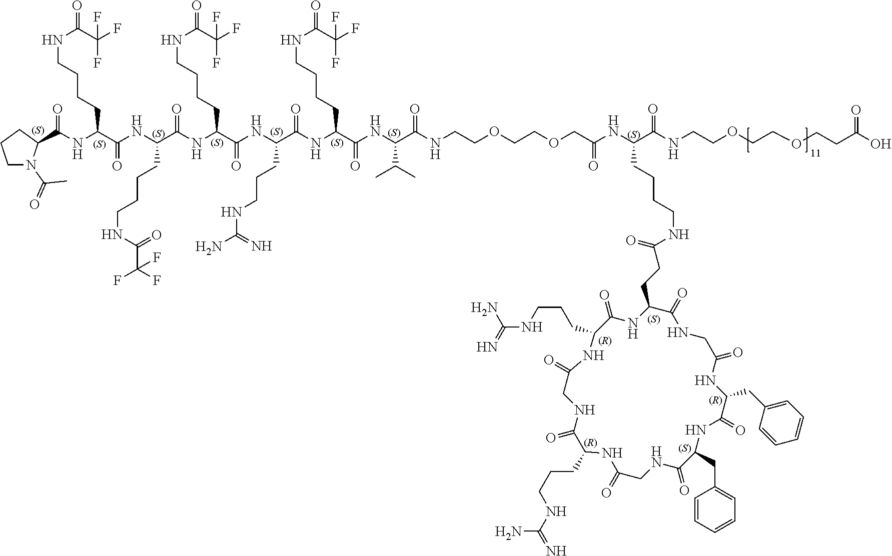

The EEV can be

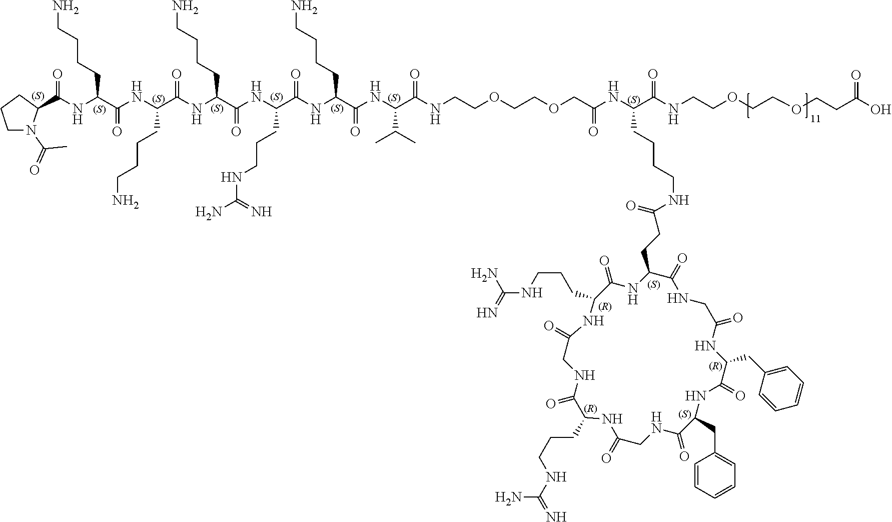

The EEV can be

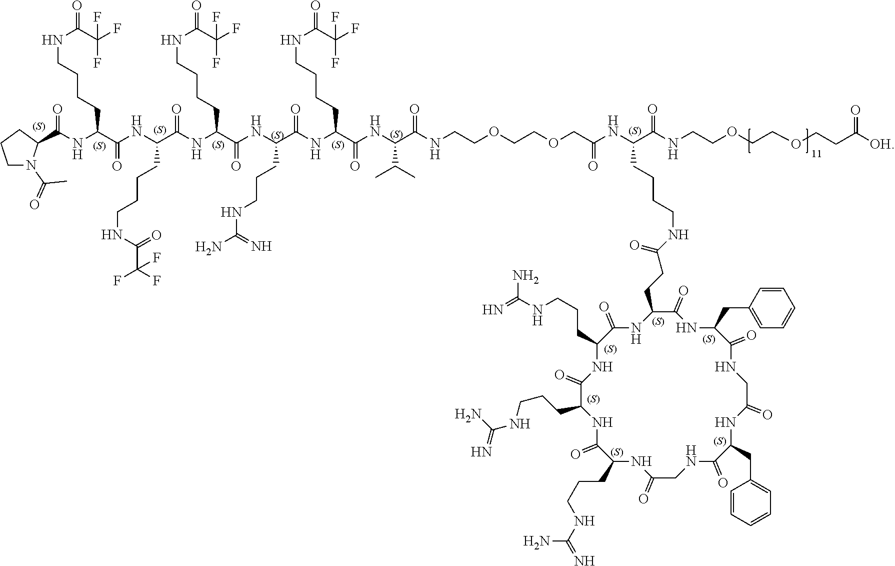

The EEV can be

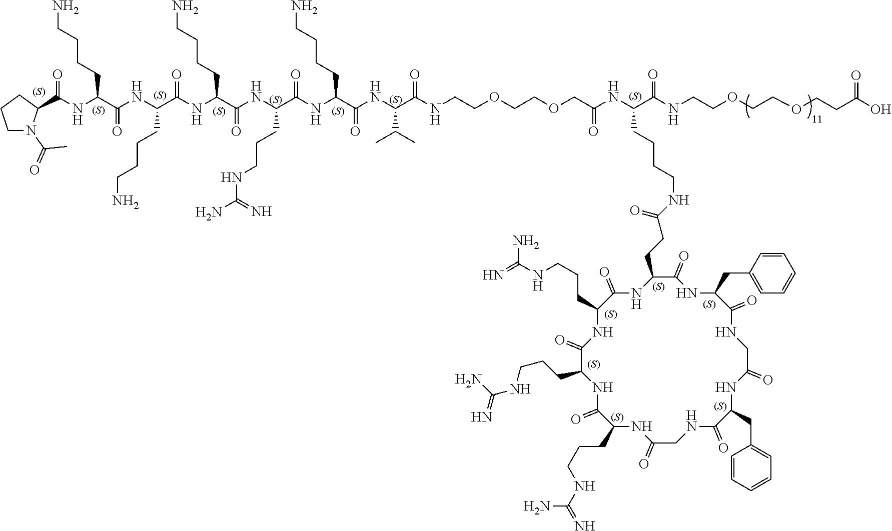

The EEV can be

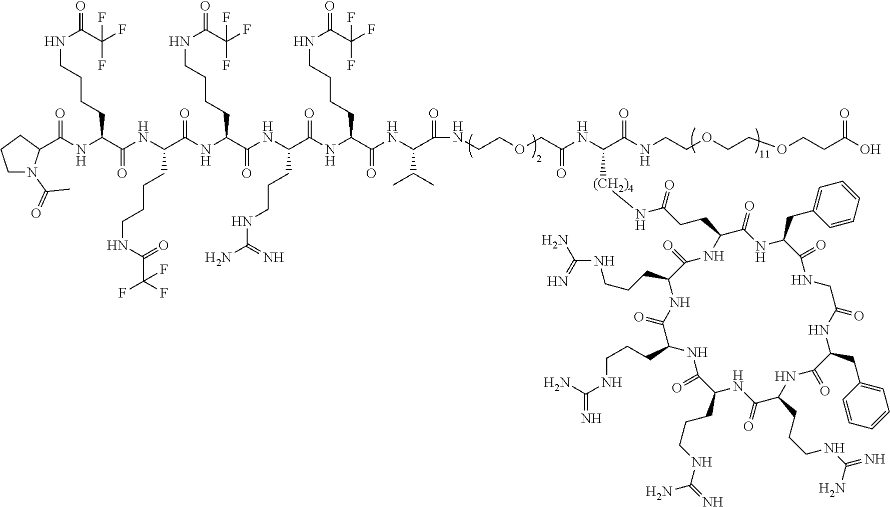

The EEV can be selected from

Ac-rr-miniPEG₂-Dap(cyclo[FfΦ-Cit-r-Cit-rQ])-PEG₁₂-OH
Ac-frr-PEG₂-Dap(cyclo[FfΦ-Cit-r-Cit-rQ])-PEG₁₂-OH
Ac-rfr-PEG₂-Dap(cyclo[FfΦ-Cit-r-Cit-rQ])-PEG₁₂-OH
Ac-rbfbr-PEG₂-Dap(cyclo[FfΦ-Cit-r-Cit-rQ])-PEG₁₂-OH
Ac-rr-PEG₂-Dap(cyclo[FfΦ-Cit-r-Cit-rQ])-PEG₁₂-OH
Ac-rbr-PEG₂-Dap(cyclo[FfΦ-Cit-r-Cit-rQ])-PEG₁₂-OH
Ac-rbrbr-PEG₂-Dap(cyclo[FfΦ-Cit-r-Cit-rQ])-PEG₁₂-OH
Ac-hh-PEG₂-Dap(cyclo[FfΦ-Cit-r-Cit-rQ])-PEG₁₂-OH
Ac-hbh-PEG₂-Dap(cyclo[FfΦ-Cit-r-Cit-rQ])-PEG₁₂-OH
Ac-hbhbh-PEG₂-Dap(cyclo[FfΦ-Cit-r-Cit-rQ])-PEG₁₂-OH
Ac-rbhbh-PEG₂-Dap(cyclo[FfΦ-Cit-r-Cit-rQ])-PEG₁₂-OH
Ac-hbrbh-PEG₂-Dap(cyclo[FfΦ-Cit-r-Cit-rQ])-PEG₁₂-OH
Ac-rr-Dap(cyclo[FfΦ-Cit-r-Cit-rQ])-b-OH
Ac-frr-Dap(cyclo[FfΦ-Cit-r-Cit-rQ])-b-OH
Ac-rfr-Dap(cyclo[FfΦ-Cit-r-Cit-rQ])-b-OH
Ac-rbfbr-Dap(cyclo[FfΦ-Cit-r-Cit-rQ])-b-OH
Ac-rrr-Dap(cyclo[FfΦ-Cit-r-Cit-rQ])-b-OH
Ac-rbr-Dap(cyclo[FfΦ-Cit-r-Cit-rQ])-b-OH
Ac-rbrbr-Dap(cyclo[FfΦ-Cit-r-Cit-rQ])-b-OH
Ac-hh-Dap(cyclo[FfΦ-Cit-r-Cit-rQ])b-OH
Ac-hbh-Dap(cyclo[FfΦ-Cit-r-Cit-rQ])-b-OH
Ac-hbhbh-Dap(cyclo[FfΦ-Cit-r-Cit-rQ])-b-OH
Ac-rbhbh-Dap(cyclo[FfΦ-Cit-r-Cit-rQ])-b-OH
Ac-hbrbh-Dap(cyclo[FfΦ-Cit-r-Cit-rQ])-b-OH
Ac-KKKK-miniPEG₂-Lys(cyclo[FfΦGrGrQ])miniPEG₂-K(N₃)-NH₂
Ac-KGKK-miniPEG₂-Lys(cyclo[FfΦGrGrQ])miniPEG₂-K(N₃)-NH₂
Ac-KKGK-miniPEG₂-Lys(cyclo[FfΦGrGrQ])-miniPEG₂-K(N₃)-NH₂
Ac-KKK-miniPEG₂-Lys(cyclo[FfΦGGrGrQ])-miniPEG₂-K(N₃)-NH₂
Ac-KK-miniPEG₂-Lys(cyclo[FfΦGrGrQ])-miniPEG₂-K(N₃)-NH₂
Ac-KGK-miniPEG₂-Lys(cyclo[FfoGrGrQ])-miniPEG₂-K(Na)-NH₂
Ac-KBK-miniPEG₂-Lys(cyclo[FfΦGGrGrQ])-miniPEG₂-K(N₃)-NH₂
Ac-KBKBK-PEG₂-Lys(cyclo[FfΦGGrGrQ])PEG₂-K(N₂)-NH₂
Ac-KR-miniPEG₂-Lys(cyclo[FfΦGGrGrQ])-miniPEG₂-K(N₃)-NH₂
Ac-KBR-miniPEG₂-Lys(cyclo[FfΦGrGrQ])miniPEG₂-K(N₃)-NH₂
Ac-PKKKRKV-miniPEG₂-Lys(cyclo[FfΦGrGrQ])miniPEG₂-K(N₃)-NH₂
Ac-PKKKRKV-miniPEG₂-Lys(cyclo[FfΦGrGrQ])miniPEG₂-K(N₃)-NH₂
Ac-PGKKRKV-miniPEG₂-Lys(cyclo[FfΦGrGrQ])miniPEG₂-K(N₃)-NH₂
Ac-PKGKRKV-miniPEG₂-Lys(cyclo[FfΦGrGrQ])miniPEG₂-K(N₃)-NH₂
Ac-PKKGRKV-miniPEG₂-Lys(cyclo[FfΦGrGrQ])miniPEG₂-K(N₃)-NH₂
Ac-PKKKGKV-miniPEG₂-Lys(cyclo[FfΦGGrGrQ])-miniPEG₂-K(N₃)-NH₂
Ac-PKKKRGV-miniPEG₂-Lys(cyclo[FfΦGrGrQ])miniPEG₂-K(N₃)-NH₂
Ac-PKKKRKG-miniPEG₂-Lys(cyclo[FfΦGrGrQ])miniPEG₂-K(N₃)-NH₂
Ac-KKKRK-miniPEG₂-Lys(cyclo[FfΦGrGrQ])miniPEG₂-K(N₃)-NH₂
Ac-KKRK-miniPEG₂-Lys(cyclo[FfΦGGrGrQ])-miniPEG₂-K(N₃)-NH₂
and
AC-KRK-miniPEG₂-Lys(cyclo[FfΦGGrGrQ])miniPEG₂-K(N₃)-NH₂.

The EEV can be selected from:

Ac-PKKKRKV-Lys(cyclo[FfΦGrGrQ])PEG₁₂-K(N₃)-NH₂
Ac-PKKKRKV-miniPEG₂-Lys(cyclo[FfΦGGrGrQ])
PEG₂-K(N₃)-NH₂
Ac-PKKKRKV-miniPEG₂-Lys(cyclo[FGFGRGRQ])
miniPEG₂-K(N₃)-NH₂
Ac-KR-PEG₂-K(cyclo[FGFGRGRQ])PEG₂-K(N₃)-NH₂
Ac-PKKKGKV-PEG₂-K(cyclo[FGFGRGRQ])PEG₂-K(N₃)-NH₂
Ac-PKKKRKG-PEG-K(cyclo[FGFGRGRQ])PEG₂-K(N₃)-NH₂
Ac-KKKRK-PEG₂-K(cyclo[FGFGRGRQ])PEG₂-K(N₃)-NH₂
Ac-PKKKRKV-miniPEG₂-Lys(cyclo[FFΦGRGRQ])
miniPEG₂-K(N₃)-NH₂
Ac-PKKKRKV-miniPEG₂-Lys(cyclo[βhFfΦGrGrQ])-
miniPEG₂-K(N₃)-NH₂
Ac-PKKKRKV-miniPEG₂-Lys(cyclo[FfΦGSrSrQ])
miniPEG₂-K(N₃)-NH₂.

The EEV can be selected from:

Ac-PKKKRKV-miniPEG₂-Lys(cyclo(GFfGrGrQ])-PEG₁₂-OH
Ac-PKKKRKV-miniPEG₂-Lys(cyclo[FGFKRKRQ])-PEG₁₂-OH
Ac-PKKKRKV-miniPEG₂-Lys(cyclo[FGFRGRGQ])-PEG₁₂-OH
Ac-PKKKRKV-miniPEG₂-Lys(cyclo[FGFGRGRGRQ])-PEG₁₂-OH
Ac-PKKKRKV-miniPEG₂-Lys(cyclo[FGFGRrRQ])-PEG₁₂-OH
Ac-PKKKRKV-miniPEG₂-Lys(cyclo[FGFGRRRQ])-PEG₁₂-OH
and
Ac-PKKKRKV-miniPEG₂-Lys(cyclo[FGFRRRRQ])-PEG₁₂-OH.

The EEV can be selected from:

Ac-K-K-K-R-K-G-miniPEG₂-K(cyclo
[FGFGRGRQ])-PEG₁₂-OH
Ac-K-K-K-R-K-miniPEG₂-K(cyclo[FGFGRGRQ])
PEG₁₂-OH
Ac-K-K-R-K-K-PEG₄-K(cyclo[FGFGRGRQ])-PEG₁₂-OH
Ac-K-R-K-K-K-PEG₄-K(cyclo[FGFGRGRQ])-PEG₁₂-OH
Ac-K-K-K-K-R-PEG₄-K(cyclo[FGFGRGRQ])-PEG₁₂-OH
Ac-R-K-K-K-K-PEG₄-K(cyclo[FGFGRGRQ])-PEG₁₂-OH
and
Ac-K-K-K-R-K-PEG₄-K(cyclo[FGFGRGRQ])-PEG₁₂-OH

The EEV can be selected from:

Ac-PKKKRKV-PEG₂-K(cyclo[FGFGRGRQ])-
PEG₂-K(N₃)-NH₂
Ac-PKKKRKV-PEG₂-K(cyclo[FGFGRGRQ])-PEG₁₂-OH
Ac-PKKKRKV-PEG₂-K(cyclo[GFfGrGrQ])-
PEG₂-K(N₃)-NH₂
and
Ac-PKKKRKV-PEG₂-K(cyclo[GFfGrGrQ])-PEG₁₂-OH .

The cargo can be a protein and the EEV can be selected from:

Ac-PKKKRKV-PEG₂-K(cyclo[FfΦGrGrQ])-PEG₁₂-OH
Ac-PKKKRKV-PEG₂-K(cyclo[FfΦCit-r-Cit-rQ])-
PEG₁₂-OH
Ac-PKKKRKV-PEG₂-K(cyclo[FFfGRGRQ])-PEG₁₂-OH
Ac-PKKKRKV-PEG₂-K(cyclo[FGFGRGRQ])-PEG₁₂-OH
Ac-PKKKRKV-PEG₂-K(cyclo[GFfGrGrQ])-PEG₁₂-OH
Ac-PKKKRKV-PEG₂-K(cyclo[FGFGRRRQ])-PEG₁₂-OH
Ac-PKKKRKV-PEG₂-K(cyclo[FGFRRRRQ])-PEG₁₂-OH
Ac-rr-PEG₂-K(cyclo[FfΦGrGrQ])-PEG₁₂-OH
Ac-rr-PEG₂-K(cyclo[FfΦCit-r-Cit-rQ])-
PEG₁₂-OH
Ac-rr-PEG₂-K(cyclo[FFfGRGRQ])-PEG₁₂-OH
Ac-rr-PEG₂-K(cyclo[FGFGRGRQ])-PEG₁₂-OH
Ac-rr-PEG₂-K(cyclo[GFfGrGrQ])-PEG₁₂-OH
Ac-rr-PEG₂-K(cyclo[FGFGRRRQ])-PEG₁₂-OH
Ac-rr-PEG₂-K(cyclo[FGFRRRRQ])-PEG₁₂-OH
Ac-rrr-PEG₂-K(cyclo[FfΦGrGrQ])-PEG₁₂-OH
Ac-rrr-PEG₂-K(cyclo[FfΦCit-r-Cit-rQ])-
PEG₁₂-OH
Ac-rrr-PEG₂-K(cyclo[FFfGRGRQ])-PEG₁₂-OH
Ac-rrr-PEG₂-K(cyclo[FGFGRGRQ])-PEG₁₂-OH
Ac-rrr-PEG₂-K(cyclo[GFfGrGrQ])-PEG₁₂-OH
Ac-rrr-PEG₂-K(cyclo[FGFGRRRQ])-PEG₁₂-OH
Ac-rrr-PEG₂-K(cyclo[FGFRRRRQ])-PEG₁₂-OH
Ac-rhr-PEG₂-K(cyclo[FfΦGrGrQ])-PEG₁₂-OH
Ac-rhr-PEG₂-K(cyclo[FfΦCit-r-Cit-rQ])-
PEG₁₂-OH
Ac-rhr-PEG₂-K(cyclo[FFfGRGRQ])-PEG₁₂-OH
Ac-rhr-PEG₂-K(cyclo[FGFGRGRQ])-PEG₁₂-OH
Ac-rhr-PEG₂-K(cyclo[GFfGrGrQ])-PEG₁₂-OH
Ac-rhr-PEG₂-K(cyclo[FGFGRRRQ])-PEG₁₂-OH
Ac-rhr-PEG₂-K(cyclo[FGFRRRRQ])-PEG₁₂-OH
Ac-rbr-PEG₂-K(cyclo[FfΦGrGrQ])-PEG₁₂-OH
Ac-rbr-PEG₂-K(cyclo[FfΦCit-r-Cit-rQ])-
PEG₁₂-OH
Ac-rbr-PEG₂-K(cyclo[FFfGRGRQ])-PEG₁₂-OH
Ac-rbr-PEG₂-K(cyclo[FGFGRGRQ])-PEG₁₂-OH
Ac-rbr-PEG₂-K(cyclo[GFfGrGrQ])-PEG₁₂-OH
Ac-rbr-PEG₂-K(cyclo[FGFGRRRQ])-PEG₁₂-OH
Ac-rbr-PEG₂-K(cyclo[FGFRRRRQ])-PEG₁₂-OH
Ac-rbrbr-PEG₂-K(cyclo[FfΦGrGrQ])-PEG₁₂-OH
Ac-rbrbr-PEG₂-K(cyclo[FfΦCit-r-Cit-rQ])-
PEG₁₂-OH
Ac-rbrbr-PEG₂-K(cyclo[FFfGRGRQ])-PEG₁₂-OH
Ac-rbrbr-PEG₂-K(cyclo[FGFGRGRQ])-PEG₁₂-OH
Ac-rbrbr-PEG₂-K(cyclo[GFfGrGrQ])-PEG₁₂-OH
Ac-rbrbr-PEG₂-K(cyclo[FGFGRRRQ])-PEG₁₂-OH
Ac-rbrbr-PEG₂-K(cyclo[FGFRRRRQ])-PEG₁₂-OH
Ac-rbhbr-PEG₂-K(cyclo[FfΦGrGrQ])-PEG₁₂-OH
Ac-rbhbr-PEG₂-K(cyclo[FfΦCit-r-Cit-rQ])-
PEG₁₂-OH
Ac-rbhbr-PEG₂-K(cyclo[FFfGRGRQ])-PEG₁₂-OH
Ac-rbhbr-PEG₂-K(cyclo[FGFGRGRQ])-PEG₁₂-OH
Ac-rbhbr-PEG₂-K(cyclo[GFfGrGrQ])-PEG₁₂-OH
Ac-rbhbr-PEG₂-K(cyclo[FGFGRRRQ])-PEG₁₂-OH
Ac-rbhbr-PEG₂-K(cyclo[FGFRRRRQ])-PEG₁₂-OH
Ac-hbrbh-PEG₂-K(cyclo[FfΦGrGrQ])-PEG₁₂-OH
Ac-hbrbh-PEG₂-K(cyclo[FfΦCit-r-Cit-rQ])-
PEG₁₂-OH
Ac-hbrbh-PEG₂-K(cyclo[FFfGRGRQ])-PEG₁₂-OH
Ac-hbrbh-PEG₂-K(cyclo[FGFGRGRQ])-PEG₁₂-OH
Ac-hbrbh-PEG₂-K(cyclo[GFfGrGrQ])-PEG₁₂-OH
Ac-hbrbh-PEG₂-K(cyclo[FGFGRRRQ])-PEG₁₂-OH
Ac-hbrbh-PEG₂-K(cyclo[FGFRRRRQ])-PEG₁₂-OH

wherein b is beta-alanine, and the exocyclic sequence can be D or L stereochemistry.

### Cargo

The cell penetrating peptide (CPP), such as a cyclic cell penetrating peptide (e.g., cCPP), can be conjugated to a cargo. As used herein, “cargo” is a compound or moiety for which delivery into a cell is desired. The cargo can be conjugated to a terminal carbonyl group of a linker. At least one atom of the cyclic peptide can be replaced by a cargo or at least one lone pair can form a bond to a cargo. The cargo can be conjugated to the cCPP by a linker. The cargo can be conjugated to an AA_{SC} by a linker. At least one atom of the cCPP can be replaced by a therapeutic moiety or at least one lone pair of the cCPP forms a bond to a therapeutic moiety. A hydroxyl group on an amino acid side chain of the cCPP can be replaced by a bond to the cargo. A hydroxyl group on a glutamine side chain of the cCPP can be replaced by a bond to the cargo. The cargo can be conjugated to the cCPP by a linker. The cargo can be conjugated to an AA_{SC} by a linker.

In embodiments, the amino acid side chain comprises a chemically reactive group to which the linker or cargo is conjugated comprises. The chemically reactive group can comprise an amine group, a carboxylic acid, an amide, a hydroxyl group, a sulfhydryl group, a guanidinyl group, a phenolic group, a thioether group, an imidazolyl group, or an indolyl group. In embodiments, the amino acid of the cCPP to which the cargo is conjugated comprises lysine, arginine, aspartic acid, glutamic acid, asparagine, glutamine, homoglutamine, serine, threonine, tyrosine, cysteine, arginine, tyrosine, methionine, histidine or tryptophan.

The cargo can comprise one or more detectable moieties, one or more degradation compounds, one or more targeting moieties, or any combination thereof. In embodiments, the cargo comprises a degradation compound.

Cyclic Cell Penetratin Peptides (cCPPs) Conjugated to a Cargo Moiety

The cyclic cell penetrating peptide (cCPP) can be conjugated to a cargo moiety.

The cargo moiety can be conjugated to the linker at the terminal carbonyl group to provide the following structure:

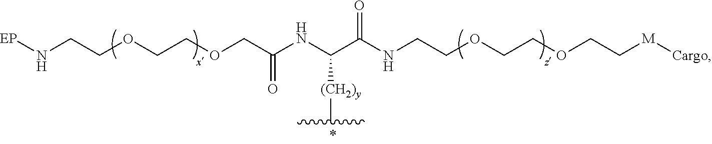

wherein:

EP is an exocyclic peptide and M, AA_{SC}, Cargo, x′, y, and z′ are as defined above, * is the point of attachment to the AA_{SC}. x′ can be 1. y can be 4. z′ can be 11. —(OCH₂CH₂)_{x′}— and/or —(OCH₂CH₂)_{z′}— can be independently replaced with one or more amino acids, including, for example, glycine, beta-alanine, 4-aminobutyric acid, 5-aminopentanoic acid, 6-aminohexanoic acid, or combinations thereof.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C):

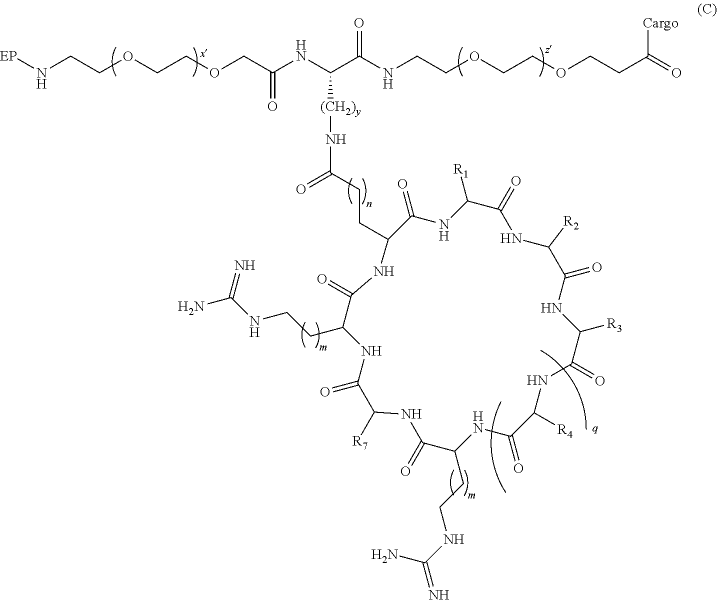

or a protonated form thereof,

wherein.
R₁, R₂, and R₃ can each independently be H or an amino acid residue having a side chain comprising an aromatic group;

R₄ is H or an amino acid side chain;

EP is an exocyclic peptide as defined herein;

Cargo is a moiety as defined herein;

each m is independently an integer from 0-3;

n is an integer from 0-2;

x′ is an integer from 2-20;

y is an integer from 1-5;

q is an integer from 1-4; and

z′ is an integer from 2-20.

R₁, R₂, R₃, R₄, EP, cargo, m, n, x′, y, q, and z′ are as defined herein.

The EEV can be conjugated to a cargo and the EEV-conjugate can comprise the structure of Formula (C-a) or (C-b):

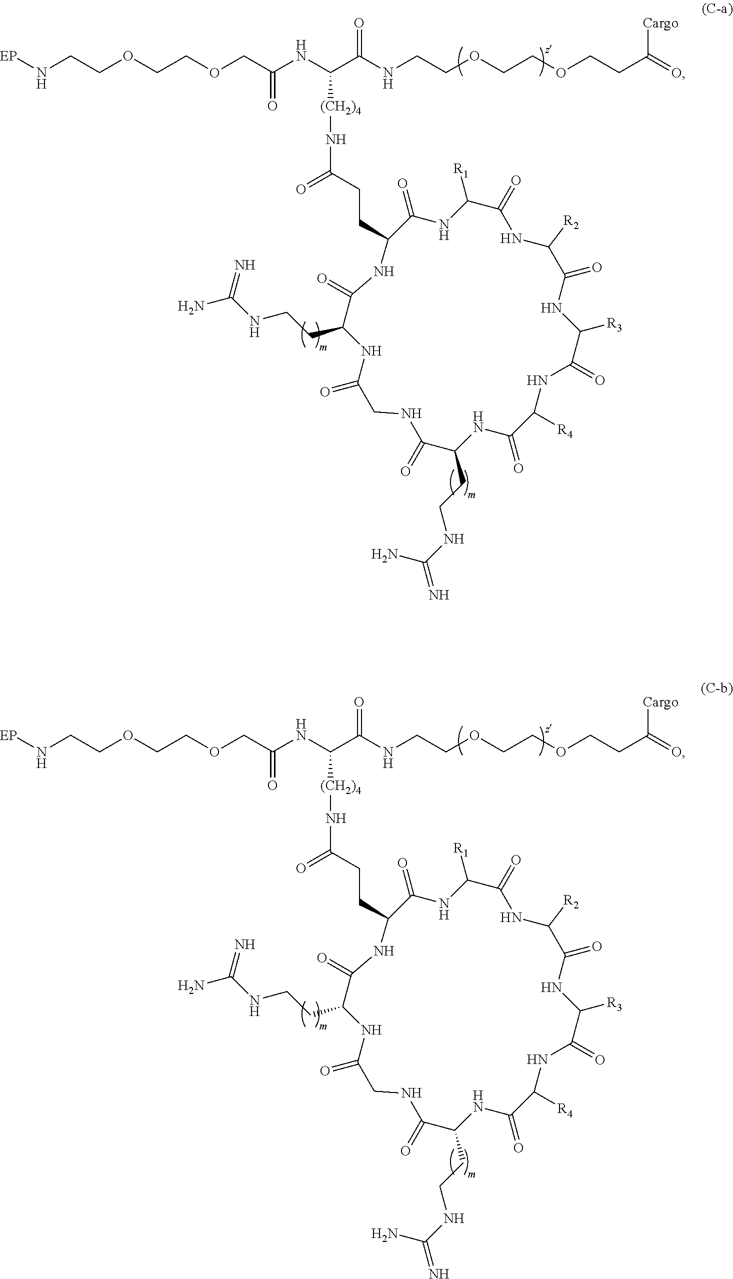

or a protonated from thereof, wherein EP, m and z are as defined above in Formula (C).

The EEV can be conjugated to a cargo and the EEV-conjugate can comprise the structure of Formula (C-c):

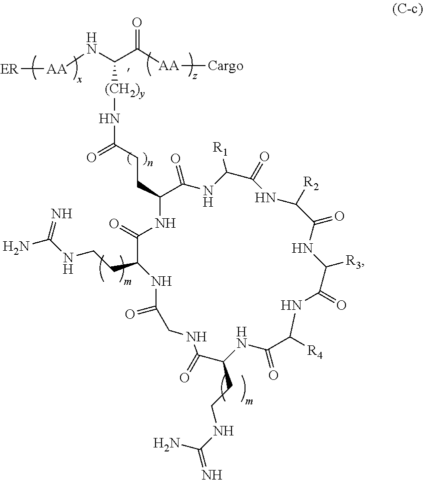

or a protonated form thereof, wherein EP, R¹, R², R³, R⁴, and m are as defined above in Formula (III); AA can be an amino acid as defined herein; n can be an integer from 0-2; x can be an integer from 1-10; y can be an integer from 1-5; and z can be an integer from 1-10.

### Cytosolic Delivery Efficiency

Modifications to a cyclic cell penetrating peptide (cCPP) may improve cytosolic delivery efficiency. Improved cytosolic uptake efficiency can be measured by comparing the cytosolic delivery efficiency of a cCPP having a modified sequence to a control sequence. The control sequence does not include a particular replacement amino acid residue in the modified sequence (including, but not limited to arginine, phenylalanine, and/or glycine), but is otherwise identical.

As used herein cytosolic delivery efficiency refers to the ability of a cCPP to traverse a cell membrane and enter the cytosol of a cell. Cytosolic delivery efficiency of the cCPP is not necessarily dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a cCPP (or a cCPP-cargo conjugate) over the concentration of the cCPP (or the cCPP-cargo conjugate) in the growth medium. Relative cytosolic delivery efficiency refers to the concentration of a cCPP in the cytosol compared to the concentration of a control cCPP in the cytosol. Quantification can be achieved by fluorescently labeling the cCPP (e.g., with a FITC dye) and measuring the fluorescence intensity using techniques well-known in the art.

Relative cytosolic delivery efficiency is determined by comparing (i) the amount of a cCPP of the invention internalized by a cell type (e.g., HeLa cells) to (ii) the amount of a control cCPP internalized by the same cell type. To measure relative cytosolic delivery efficiency, the cell type may be incubated in the presence of a cCPP for a specified period of time (e.g., 30 minutes, 1 hour, 2 hours, etc.) after which the amount of the cCPP internalized by the cell is quantified using methods known in the art, e.g., fluorescence microscopy. Separately, the same concentration of the control cCPP is incubated in the presence of the cell type over the same period of time, and the amount of the control cCPP internalized by the cell is quantified.

Relative cytosolic delivery efficiency can be determined by measuring the IC₅₀ of a cCPP having a modified sequence for an intracellular target and comparing the IC₅₀ of the cCPP having the modified sequence to a control sequence (as described herein).

The relative cytosolic delivery efficiency of the cCPPs can be in the range of from about 50% to about 450% compared to cyclo(FfΦRrRrQ), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, inclusive of all values and subranges therebetween. The relative cytosolic delivery efficiency of the cCPPs can be improved by greater than about 600% compared to a cyclic peptide comprising cyclo(FfΦRrRrQ).

The absolute cytosolic delivery efficacy of from about 40% to about 100%, e.g., about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, inclusive of all values and subranges therebetween.

The cCPPs of the present disclosure can improve the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), Sigma (St. Louis, Mo.), Pfizer (New York, N.Y.), GlaxoSmithKline (Raleigh, N.C.), Merck (Whitehouse Station, N.J.), Johnson & Johnson (New Brunswick, N.J.), Aventis (Bridgewater, N.J.), AstraZeneca (Wilmington, Del.), Novartis (Basel, Switzerland), Wyeth (Madison, N.J.), Bristol-Myers-Squibb (New York, N.Y.), Roche (Basel, Switzerland), Lilly (Indianapolis, Ind.), Abbott (Abbott Park, Ill.), Schering Plough (Kenilworth, N.J.), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for aspartic acid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl)phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine.

Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivatized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

The above polymers, such as PEG groups, can be attached to an oligonucleotide, such as an AC, under any suitable conditions. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the AC (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., α-iodo acetic acid, α-bromoacetic acid, α-chloroacetic acid). If attached to the AC by reductive alkylation, the polymer selected should have a single reactive aldehyde so that the degree of polymerization is controlled. See, for example, Kinstler et al., Adv. Drug. Delivery Rev. (2002), 54: 477-485; Roberts et al., Adv. Drug Delivery Rev. (2002), 54: 459-476; and Zalipsky et al., Adv. Drug Delivery Rev. (1995), 16: 157-182.

In order to direct covalently link the degrader compound or linker to the CPP, appropriate amino acid residues of the CPP may be reacted with an organic derivatizing agent that is capable of reacting with a selected side chain or the N- or C-termini of an amino acids. Reactive groups on the peptide or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art.

Non-limiting examples of compounds that include a CPPs and a reactive group useful for conjugation to a degrader compound or a linker are shown in Table 18. Example linker groups are also shown. Example reactive groups include tetrafluorophenyl ester (TFP), free carboxylic acid (COOH), and azide (N₃). In Table 18, n is an integer from 0 to 20; Pipa6 is AcRXRRBRRXRYQFLIRXRBRXRB wherein B is β-Alanine and X is aminohexanoic acid; Dap is 2,3-diaminopropionic acid; NLS is a nuclear localization sequence; βA is beta alanine; -ss- is a disulfide; PABC is poly(A) binding protein C-terminal domain; Cₓ where x is a number is an alkyl chain of length x; and BCN is bicyclo [6.1.0]nonyne.

TABLE 18
Compounds that include a CPPs and a reactive group

TFP-PEGₙ-K(CPP)
TFP-PEGₙ-K(CPP)-PEGₙ-Dap(palmitoyl)
TFP-PEGₙ-K(CPP)-PEGₙ-Dap(CPP)
TFP-Pip6a
CPP-PEGₙ-TFP
CPP-PEGₙ-K(CPP)-PEGₙ-TFP
CPP-PEGₙ-Lys(N₃)
CPP-K(CPP)-PEGₙ-K(N₃)
CPP-PEGₙ-K(PEGₙ-CPP)-PEGₙ-K(N₃)
CPP-PEGₙ-K(PEGₙ-CPP)-PEGₙ-K(N₃)
CPP-K(CPP)-K(CPP)-PEGₙ-K(N₃)
CPP-PEGₙ-K(PEGₙ-CPP)-K(PEGₙ-CPP)-PEGₙ-K(N₃)
CPP-PEGₙ-K(PEGₙ-CPP)-K(PEGₙ-CPP)-PEGₙ-K(N₃)
Ac-NLS-Lys(CPP)-PEGₙ-K(N₃)
K(N₃)-PEGₙ-NLS-ss-PEGₙ-CPP
BCN-NLS-ss-CPP
CPP-PEGₙ-Val-Cit-PABC-K(N₃)
CPP-PEGₙ-Cys-ss-Cys-K(N₃)
CPP-PEGₙ-Cys-ss-Cys-K(N₃)
CPP-PEGₙ-TFP
CPP-PEGₙ-Lys(N)
CPP-PEGₙ-Cys-prodisulfide-K(N2)
CPP-PEGₙ-K(N₃)
CPP-K(CPP)-PEGₙ-K(N₃)
CPP-PEGₙ-K(CPP)-PEGₙ-TFP
CPP-C6-TFP
CPP-PEGₙ-K(PEGₙ-CPP)PEGₙ-K(N₃)
Ac-T9-PEGₙ-Lys(CPP-PEGₙ)-K(N₃)
Ac-MSP-PEGₙ-K(CPP-PEGₙ)-K(N₃)
CPP-PEGₙ-TFP (ENTRD 802)
CPP-C6-TFP (ENTRD 696)
CPP-PEGₙ-K(CPP)-PEGₙ-TFP (ENTRD-344)
CPP-PEGₙ-COOH
CPP-C12-TFP (ENTD-695)
palmitoyl-PEGₙ-K(CPP)-PEGₙ-TFP (ENTD-343)
CPP-PEGₙ-K(N₃) (ENTRD-617)
Ac-T9-PEGₙ-K(CPP)-K(N₃) (ENTRD 673)
Ac-MSP-PEGₙ-K(CPP-PEGₙ)-K(N₃) (ENTRD 675)
Ac-NLS-K(CPP)-PEGₙ-K(N₃) (ENTRD 684)
K(N3)-PEGₙ-NLS-ss-PEGₙ-CPP (ETRD-681)
K(N3)-PEGₙ-NLS-K-βA-βA-CPP (ETRD-682)

In embodiments, the CPPs have free carboxylic acid groups that may be utilized for conjugation to a degrader compound or a linker. The CPPs may have free amide groups that may be utilized for conjugation to a degrader compound or a linker. The CPPS may have cyclooctyne or azide groups to conjugate to an appropriately modified degrader compound or linker via strain-promoted azide-alkyne cycloaddition. The CPPs and/or the degrader compounds may comprise linkers containing suitable functional groups to permit conjugation.

### Polynucleotides and Expression Vectors

Provided herein are nucleic acid molecules comprising a nucleic acid sequence encoding a degradation construct, degradation moiety, and/or degradation moiety described herein. The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded and double-stranded polynucleotides.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% r less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added, deleted, or replaced with different nucleotides compared to a reference polynucleotide. In this regard, it is well understood in the art that certain alterations inclusive of mutations, additions, deletions, and substitutions can be made to a reference polynucleotide whereby the altered polynucleotide retains the biological function or activity of the reference polynucleotide. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% r 100% sequence identity to a reference sequence.

Examples of some polynucleotides encoding the degradation constructs described herein are provided below in Table 19. Construct details are written 5′ to 3′. Although no linker (sequence encoding a polypeptide linker) is not explicitly included in the construct details, a linker may be present a person of ordinary skill in the art would be able to determine the linker via methods known in the art. Additionally, N terminal and or C terminal protein tags are not shown but may be encoded by the polynucleotides and a person or ordinary skill in the art would be able to determine a protein tag sequence via methods known in the art.

TABLE 19
Examples of some Polynucleotides Encoding Degradation Constructs.

| SEQ ID | Construct | |
|---|---|---|
| NO | Specifics | Sequence |
| 323 | ASC^{VHH}_ | CAGTTGCAGCTAGTTGAAAGTGGTGGCGGTTTAGTTCAGCCAGGCG |
| | hFc | GTTCATTAAAGTTATCATGTGCTGCTTCAGGCTTCACCTTTTCAAG |
| | (N297A) | ATACGCCATGAGTTGGTACCGTCAAGCCCCTGGAAAGGAGAGAGAG |
| | | TCTGTTGCTAGGATTTCATCTGGCGGAGGTACGATCTACTACGCTG |
| | | ATTCAGTCAAAGGTAGGTTCACCATAAGTAGGGAGGACGCAAAGAA |
| | | CACCGTTTATCTGCAGATGAACTCCCTTAAACCTGAGGACACGGCC |
| | | GTCTACTACTGTTATGTAGGCGGCTTTTGGGGCCAAGGTACTCAGG |
| | | TCACTGTTAGTTCCGGAGGAGGAGGATCTGATAAAACCCATACTTG |
| | | TCCTCCCTGTCCCGCACCTGAGCTTTTAGGAGGTCCTTCCGTATTT |
| | | TTATTTCCCCCTAAGCCTAAAGATACTCTGATGATTAGTCGTACTC |
| | | CTGAGGTTACGTGTGTTGTAGTCGATGTATCCCACGAGGACCCAGA |
| | | AGTTAAGTTTAATTGGTATGTAGACGGCGTGGAGGTCCACAACGCC |
| | | AAAACTAAGCCCAGGGAAGAGCAGTATGCCAGTACATATAGGGTAG |
| | | TCTCAGTACTGACCGTGCTACACCAGGATTGGTTGAATGGAAAAGA |
| | | ATATAAATGTAAAGTGTCTAATAAGGCACTGCCCGCTCCAATCGAG |
| | | AAGACGATTTCCAAAGCCAAGGGCCAGCCCAGAGAGCCCCAGGTTT |
| | | ATACTTTGCCACCAAGTCGTGAAGAAATGACAAAGAATCAGGTATC |
| | | ACTGACGTGTCTGGTTAAAGGTTTCTATCCTAGTGACATAGCCGTC |
| | | GAGTGGGAATCTAATGGTCAACCAGAGAATAATTATAAGACTACTC |
| | | CACCAGTATTAGACAGTGACGGTTCTTTCTTTCTTTACAGTAAACT |
| | | GACAGTAGACAAAAGTCGTTGGCAACAGGGAAATGTCTTTTCATGC |
| | | TCAGTGATGCATGAGGCCCTTCACAACCATTACACCCAAAAATCAC |
| | | TATCCTTGAGTCCCGGCTGC |
| 324 | ASC^{VHH}_ | CAATTGCAGTTGGTGGAATCAGGCGGAGGTTTGGTTCAACCTGGTG |
| | hFc(N297A/ | GTTCTCTAAAATTATCTTGTGCTGCAAGTGGATTCACATTTAGTCG |
| | H433A) | TTACGCTATGTCATGGTATAGACAGGCACCAGGAAAAGAGAGGGAG |
| | | AGTGTCGCAAGAATCAGTTCTGGAGGCGGTACGATTTACTACGCTG |
| | | ACTCAGTGAAGGGCAGGTTTACTATTTCTAGGGAAGACGCTAAAAA |
| | | CACCGTATACCTACAAATGAACTCCCTGAAGCCCGAGGATACTGCA |
| | | GTTTACTATTGTTACGTTGGAGGCTTTTGGGGACAGGGCACCCAGG |
| | | TTACTGTTTCTTCAGGTGGCGGTGGTTCCGATAAGACACACACGTG |
| | | CCCCCCATGTCCCGCACCAGAACTTCTAGGCGGTCCCTCCGTATTC |
| | | TTATTCCCACCCAAACCTAAGGATACATTGATGATCTCAAGGACGC |
| | | CCGAGGTCACATGCGTGGTGGTGGACGTTAGTCACGAAGACCCAGA |
| | | AGTAAAGTTCAATTGGTACGTAGACGGTGTCGAGGTACACAATGCC |
| | | AAAACAAAGCCAAGAGAGGAGCAATACGCATCTACCTATAGAGTAG |
| | | TTAGTGTCTTGACCGTGTTGCATCAAGATTGGCTTAACGGTAAAGA |
| | | GTACAAGTGCAAGGTTAGTAATAAGGCCTTGCCCGCTCCAATTGAA |
| | | AAGACAATCTCCAAAGCCAAAGGTCAGCCTCGTGAGCCCCAAGTTT |
| | | ACACCTTACCACCATCTCGTGAGGAGATGACTAAGAATCAAGTAAG |
| | | TTTGACTTGTCTAGTGAAAGGCTTTTACCCTAGTGACATAGCCGTC |
| | | GAGTGGGAATCAAACGGCCAGCCAGAGAATAATTATAAGACAACGC |
| | | CCCCCGTTCTTGACTCCGATGGTTCCTTTTTTTTATATTCAAAGCT |
| | | AACGGTTGACAAGTCCAGATGGCAACAAGGTAACGTCTTTAGTTGT |
| | | AGTGTGATGCACGAAGCCCTTGCAAATCATTATACGCAAAAATCAT |
| | | TATCCCTGAGTCCCGGATGT |
| 325 | ASC^{VHH}_ | CAACTTCAGCTGGTGGAATCCGGAGGAGGTCTTGTCCAACCAGGAG |
| | hFc(N297A/ | GATCATTGAAGTTGTCATGCGCTGCATCCGGCTTTACCTTTAGTCG |
| | S239C) | TTATGCAATGTCCTGGTATAGACAAGCACCTGGTAAGGAGAGGGAG |
| | | TCAGTTGCCAGGATCAGTAGTGGAGGTGGAACTATTTACTATGCTG |
| | | ATTCTGTCAAGGGCCGTTTCACAATTTCCCGTGAGGATGCCAAGAA |
| | | TACAGTATATTTGCAAATGAACTCCCTGAAACCAGAGGATACTGCA |
| | | GTATACTATTGTTACGTGGGTGGTTTCTGGGGACAGGGAACGCAAG |
| | | TGACGGTATCATCAGGTGGAGGCGGTTCCGACAAAACACACACCTG |
| | | TCCTCCCTGCCCCGCCCCTGAATTATTGGGAGGACCATGCGTATTT |
| | | CTATTCCCTCCAAAGCCCAAGGACACATTAATGATCTCTCGTACGC |
| | | CTGAAGTGACTTGCGTGGTAGTTGACGTATCACACGAGGATCCTGA |
| | | AGTAAAGTTTAATTGGTACGTGGACGGTGTCGAAGTTCATAATGCT |
| | | AAGACGAAGCCCAGAGAAGAGCAATACGCTTCCACGTACAGGGTCG |
| | | TATCCGTACTAACAGTTCTACACCAAGATTGGCTGAATGGAAAAGA |
| | | GTATAAATGCAAGGTGTCCAACAAGGCATTGCCTGCCCCCATAGAA |
| | | AAGACGATATCAAAAGCTAAGGGTCAACCAAGAGAGCCTCAAGTAT |
| | | ACACACTACCACCCAGTAGGGAAGAGATGACTAAAAACCAGGTGTC |
| | | CCTAACCTGCCTGGTGAAAGGCTTCTATCCAAGTGACATCGCCGTC |
| | | GAGTGGGAATCTAATGGACAACCAGAGAATAATTACAAGACAACAC |
| | | CTCCTGTCCTGGATTCCGATGGCTCTTTTTTCTTGTATTCCAAGTT |
| | | GACTGTTGACAAGTCAAGGTGGCAGCAGGGAAACGTCTTTTCTTGC |
| | | AGTGTTATGCACGAAGCTCTTCATAACCATTACACTCAGAAATCCC |
| | | TGAGTCTTTCCCCTGGTAAG |
| 326 | BC2- | CAGGTGCAACTTGTTGAGTCAGGTGGTGGTCTAGTACAACCTGGCG |
| | hFc(N297A) | GTAGTCTTACGTTATCCTGTACTGCCTCAGGATTTACGCTAGATCA |
| | | CTACGACATAGGCTGGTTTAGGCAAGCACCTGGAAAGGAAAGAGAA |
| | | GGCGTGTCTTGCATTAACAATAGTGACGATGACACTTATTATGCCG |
| | | ACTCCGTCAAAGGAAGATTCACAATCTTTATGAACAATGCAAAAGA |
| | | CACTGTTTACCTGCAGATGAACAGTTTAAAGCCCGAGGATACGGCT |
| | | ATATATTACTGCGCCGAAGCCAGGGGCTGTAAAAGGGGAAGATACG |
| | | AATACGATTTCTGGGGCCAAGGCACGCAAGTTACCGTCTCTTCCGG |
| | | AGGAGGAGGTTCTGACAAAACTCATACATGCCCTCCTTGTCCCGCA |
| | | CCTGAGTTGCTTGGCGGCCCTTCCGTTTTCCTGTTTCCACCAAAAC |
| | | CAAAAGACACACTTATGATTAGTCGTACACCCGAGGTTACGTGCGT |
| | | AGTGGTTGATGTGTCACATGAGGACCCCGAGGTTAAGTTCAACTGG |
| | | TATGTTGATGGAGTGGAGGTGCATAATGCCAAAACGAAACCCAGGG |
| | | AGGAGCAGTATGCTTCCACGTATAGGGTAGTTAGTGTTCTGACTGT |
| | | TTTACATCAAGATTGGTTGAACGGCAAGGAGTATAAATGTAAAGTC |
| | | TCAAATAAAGCTCTTCCTGCTCCCATCGAGAAAACTATCTCCAAGG |
| | | CCAAAGGACAACCTCGTGAACCCCAAGTGTACACACTTCCACCTAG |
| | | TCGTGAGGAAATGACTAAGAACCAGGTGTCTTTAACGTGTCTAGTA |
| | | AAAGGATTCTACCCATCCGATATTGCAGTTGAATGGGAGTCCAATG |
| | | GACAGCCAGAGAACAACTACAAAACTACCCCACCCGTCCTTGATTC |
| | | AGACGGATCATTTTTTCTTTACTCCAAATTAACAGTCGATAAAAGT |
| | | AGGTGGCAGCAGGGAAATGTTTTCTCATGTAGTGTAATGCACGAAG |
| | | CACTACACAATCACTATACTCAAAAAAGTCTGTCATTAAGTCCTGG |
| | | CTGT |
| 327 | BC2-hFc | CAAGTTCAGCTAGTGGAGTCCGGCGGCGGCCTTGTTCAGCCAGGAG |
| | (N297A/ | GTTCACTAACGCTGTCCTGCACAGCATCCGGCTTCACGTTAGACCA |
| | H433A) | TTACGATATTGGATGGTTCAGACAGGCTCCAGGAAAAGAGAGAGAG |
| | | GGCGTTTCTTGTATCAATAACTCAGACGACGACACTTATTATGCAG |
| | | ATTCCGTTAAAGGTAGGTTTACAATATTTATGAATAATGCCAAAGA |
| | | CACGGTGTATTTGCAAATGAACTCTTTAAAGCCCGAGGACACGGCC |
| | | ATCTACTACTGCGCTGAGGCAAGAGGCTGTAAGAGAGGAAGATATG |
| | | AATATGACTTCTGGGGACAAGGCACCCAGGTTACGGTCTCAAGTGG |
| | | CGGCGGTGGCAGTGACAAGACGCACACTTGTCCTCCCTGCCCCGCT |
| | | CCTGAATTATTGGGCGGTCCATCTGTTTTCCTTTTCCCCCCAAAGC |
| | | CTAAAGATACACTGATGATATCCCGTACGCCCGAGGTGACATGCGT |
| | | CGTTGTCGACGTGTCACATGAGGACCCTGAGGTTAAATTTAACTGG |
| | | TACGTAGATGGAGTAGAGGTACATAACGCAAAAACGAAGCCAAGAG |
| | | AGGAACAATACGCCTCCACTTACCGTGTCGTATCCGTTTTGACGGT |
| | | CTTGCATCAAGACTGGTTAAACGGCAAAGAATACAAGTGCAAAGTT |
| | | TCCAATAAGGCACTGCCAGCTCCTATTGAAAAGACGATTTCTAAGG |
| | | CAAAGGGTCAACCTCGTGAGCCTCAAGTCTACACCCTGCCACCATC |
| | | ACGTGAAGAAATGACAAAAAACCAAGTTTCTCTGACTTGTCTGGTG |
| | | AAGGGCTTTTATCCCTCTGACATTGCCGTGGAATGGGAGTCCAACG |
| | | GACAGCCAGAGAATAACTATAAAACAACGCCACCCGTTCTGGACTC |
| | | AGACGGATCATTTTTTTTATATTCCAAATTGACTGTGGACAAGAGT |
| | | CGTTGGCAACAGGGTAACGTATTTTCTTGTTCCGTGATGCACGAAG |
| | | CTCTGGCCAACCATTACACGCAAAAATCTTTGTCACTATCACCTGG |
| | | TTGC |
| 328 | KRas^{VHH}_ | GAGGTGCAGTTGGTCGAGTCCGGTGGTGGTTCTGTACAAACCGGTG |
| | hFc(N297A) | GTTCACTTCGTCTGTCATGCGCCGTTTCAGGTAACATAGGTTCCTC |
| | | ATATTGCATGGGATGGTTTAGGCAAGCCCCTGGAAAAAAGAGAGAA |
| | | GCAGTAGCCAGAATAGTACGTGATGGTGCTACGGGCTACGCTGATT |
| | | ACGTTAAAGGCCGTTTCACAATTTCAAGGGACTCCGCTAAGAACAC |
| | | TCTTTATCTGCAAATGAATAGGCTTATCCCAGAGGATACTGCCATC |
| | | TACTATTGTGCTGCCGATCTTCCCCCAGGCTGCCTAACCCAGGCCA |
| | | TCTGGAACTTTGGATACAGGGGCCAGGGCACCTTGGTTACCGTCTC |
| | | AAGTGGCGGTGGTGGAAGTGATAAAACTCACACTTGCCCTCCTTGC |
| | | CCAGCCCCAGAGCTTCTAGGTGGTCCTTCAGTTTTTTTATTTCCCC |
| | | CTAAGCCCAAGGACACGTTGATGATTTCAAGAACCCCAGAGGTTAC |
| | | TTGTGTCGTAGTAGACGTTAGTCACGAGGACCCCGAGGTGAAGTTC |
| | | AATTGGTACGTCGACGGCGTTGAGGTACATAATGCTAAAACCAAAC |
| | | CACGTGAAGAGCAGTATGCCTCTACCTACCGTGTAGTGTCTGTTTT |
| | | AACTGTATTGCATCAGGACTGGTTAAACGGTAAAGAGTATAAATGC |
| | | AAGGTCTCTAATAAGGCCTTGCCAGCACCAATTGAGAAGACCATAT |
| | | CCAAGGCAAAAGGCCAACCCAGAGAGCCACAAGTTTACACATTGCC |
| | | TCCTTCACGTGAAGAAATGACCAAAAATCAGGTGTCTTTGACATGT |
| | | TTGGTAAAGGGCTTTTACCCCAGTGACATTGCTGTGGAGTGGGAGA |
| | | GTAATGGACAGCCCGAAAATAACTACAAAACGACTCCACCCGTGCT |
| | | TGATAGTGACGGTTCTTTCTTCCTATACTCCAAACTGACTGTCGAC |
| | | AAATCAAGATGGCAACAGGGAAATGTTTTCAGTTGCAGTGTCATGC |
| | | ACGAAGCACTGCATAATCACTACACCCAAAAATCATTGTCCTTAAG |
| | | TCCCGGTTGC |
| 329 | KRas^{VHH}_ | GAGGTACAACTTGTGGAGTCAGGCGGTGGCTCCGTGCAAACAGGCG |
| | hFc | GTAGTCTAAGACTTTCATGTGCAGTATCCGGCAACATTGGCTCTAG |
| | (N297A/ | TTATTGCATGGGATGGTTCAGGCAAGCACCCGGAAAGAAGCGTGAG |
| | H433A) | GCCGTAGCACGTATTGTCCGTGACGGAGCTACTGGTTACGCAGACT |
| | | ACGTAAAGGGTCGTTTTACCATAAGTCGTGATTCAGCTAAAAACAC |
| | | TCTTTACCTACAAATGAACCGTCTAATCCCAGAAGACACCGCAATC |
| | | TATTATTGCGCCGCAGATTTGCCCCCTGGATGCTTGACTCAAGCAA |
| | | TCTGGAATTTTGGATATAGAGGACAGGGTACCTTGGTAACCGTTTC |
| | | ATCAGGCGGCGGTGGTTCCGATAAGACGCATACCTGTCCCCCATGT |
| | | CCTGCACCTGAGTTACTAGGTGGACCTTCAGTTTTTTTATTTCCAC |
| | | CAAAACCTAAAGATACTTTAATGATAAGTCGTACCCCAGAAGTCAC |
| | | GTGTGTCGTTGTTGATGTTTCACACGAGGATCCTGAAGTGAAATTT |
| | | AATTGGTATGTTGACGGCGTCGAAGTACATAATGCAAAAACAAAAC |
| | | CAAGAGAAGAGCAGTATGCTTCTACATACAGGGTGGTAAGTGTGCT |
| | | AACCGTACTACACCAAGATTGGTTAAACGGTAAGGAATATAAATGT |
| | | AAAGTTTCTAACAAGGCTCTGCCAGCTCCCATAGAAAAGACTATTT |
| | | CCAAGGCCAAGGGACAACCCAGAGAACCCCAAGTATATACCCTTCC |
| | | ACCTTCTAGGGAAGAGATGACCAAGAATCAGGTGTCACTAACCTGT |
| | | CTAGTCAAGGGCTTTTATCCTTCTGATATAGCTGTAGAGTGGGAAT |
| | | CTAATGGCCAGCCTGAGAACAATTACAAAACTACCCCACCCGTGCT |
| | | TGATAGTGATGGATCATTCTTCCTTTACTCAAAGCTGACCGTGGAC |
| | | AAAAGTAGGTGGCAGCAGGGTAACGTCTTCTCCTGCAGTGTAATGC |
| | | ATGAAGCCTTAGCAAATCATTATACACAAAAATCCTTATCACTGTC |
| | | ACCTGGCTGT |
| 330 | BC2-Mb- | CAGGTTCAGCTGGTCGAGTCCGGCGGTGGACTGGTCCAACCAGGTG |
| | Cys | GATCTCTTACACTGAGTTGTACGGCCTCTGGATTTACGCTGGACCA |
| | | CTACGACATTGGATGGTTTAGGCAAGCACCAGGTAAAGAGAGGGAG |
| | | GGTGTTTCCTGCATAAATAATTCCGATGATGATACTTACTATGCTG |
| | | ACTCTGTGAAAGGTAGGTTCACGATCTTTATGAACAATGCCAAAGA |
| | | CACTGTGTACTTACAAATGAACTCATTGAAACCAGAGGATACCGCT |
| | | ATTTACTACTGCGCTGAAGCCCGTGGCTGTAAGAGGGGTCGTTACG |
| | | AGTATGACTTCTGGGGACAGGGAACTCAAGTAACGGTCTCATCAGA |
| | | ACCCAAGTCTTCTGATAAGACGCACACATGCCCCCCTTGTCCCGGA |
| | | CAGCCAAGGGAGCCACAAGTCTACACATTACCCCCATCCAGAGAAG |
| | | AGATGACCAAGAATCAAGTATCCTTAACCTGCTTGGTTAAGGGATT |
| | | TTACCCTTCAGATATAGCAGTAGAATGGGAATCTAATGGCCAACCA |
| | | GAAAACAACTATAAAACCACGCCACCAGTTCTGGACTCCGATGGTT |
| | | CCTTCTTCCTATACAGTAAATTGACCGTTGATAAATCCAGATGGCA |
| | | ACAAGGCAACGTGTTCAGTTGCTCAGTTATGCATGAGGCTTTGCAT |
| | | AATCATTATACTCAGAAATCTTTGTCCCTTTCCCCCGGATGC |
| 331 | 7D12-Mb- | CAAGTTAAACTGGAAGAGTCAGGTGGCGGTTCAGTACAGACCGGTG |
| | Cys | GTTCACTTCGTCTAACTTGTGCAGCAAGTGGAAGAACCTCCCGTTC |
| | | TTACGGTATGGGTTGGTTTCGTCAAGCACCCGGTAAGGAACGTGAA |
| | | TTTGTTAGTGGTATAAGTTGGAGAGGCGACTCTACGGGCTATGCCG |
| | | ATAGTGTCAAAGGTAGGTTCACTATTTCTAGAGATAATGCTAAAAA |
| | | CACCGTCGACTTGCAAATGAACTCATTGAAACCAGAAGATACGGCT |
| | | ATATATTACTGTGCTGCCGCCGCTGGATCAGCATGGTACGGCACAC |
| | | TGTACGAATATGACTACTGGGGCCAAGGCACGCAGGTAACCGTGTC |
| | | CAGTGAACCCAAGTCATCCGACAAAACCCACACCTGTCCTCCATGC |
| | | CCCGGCCAACCAAGGGAACCACAAGTATATACGTTACCTCCAAGTC |
| | | GTGAGGAGATGACTAAGAACCAGGTTTCCCTGACCTGCCTGGTTAA |
| | | AGGTTTCTACCCAAGTGACATCGCAGTTGAATGGGAATCCAATGGT |
| | | CAGCCTGAGAACAATTATAAGACTACCCCTCCTGTCCTAGATAGTG |
| | | ACGGTTCATTTTTCTTATACTCTAAATTGACCGTAGATAAAAGTAG |
| | | ATGGCAACAAGGCAATGTTTTTTCATGCTCTGTAATGCACGAGGCT |
| | | CTTCACAACCACTACACACAAAAGAGTCTTTCTCTGTCCCCAGGCT |
| | | GT |
| 332 | ASC^{VHH}_ | CAACTACAATTAGTGGAATCTGGAGGCGGTCTTGTGCAACCCGGCG |
| | Mb-Cys | GTTCCTTAAAATTGTCCTGCGCTGCCTCAGGATTTACTTTTAGTAG |
| | | ATATGCCATGTCATGGTACAGGCAGGCTCCAGGCAAGGAACGTGAA |
| | | AGTGTGGCAAGAATATCTAGTGGAGGAGGAACGATATATTACGCAG |
| | | ATTCCGTAAAAGGCAGATTTACTATTAGTCGTGAAGATGCAAAAAA |
| | | TACAGTATACCTTCAGATGAACTCTTTGAAACCAGAAGATACGGCC |
| | | GTTTATTATTGTTACGTTGGAGGATTTTGGGGACAGGGAACCCAGG |
| | | TCACAGTAAGTTCCGAGCCTAAATCTAGTGATAAAACGCATACTTG |
| | | TCCACCTTGTCCAGGCCAGCCTCGTGAGCCCCAAGTTTATACGCTT |
| | | CCTCCCTCTCGTGAAGAAATGACAAAAAATCAGGTCTCTCTAACCT |
| | | GCCTTGTAAAGGGATTTTACCCATCTGACATTGCCGTCGAATGGGA |
| | | AAGTAACGGACAGCCCGAGAATAATTATAAAACGACACCTCCCGTC |
| | | TTAGATTCTGATGGCTCTTTCTTTTTATACAGTAAGCTGACTGTGG |
| | | ACAAGAGTAGATGGCAACAGGGTAATGTTTTCTCTTGTTCAGTCAT |
| | | GCATGAAGCCTTGCATAACCATTATACCCAAAAATCTCTTAGTCTT |
| | | TCTCCCGGATGT |
| 333 | KRas^{VHH}_ | GAGGTACAGTTAGTGGAGTCTGGTGGTGGCTCCGTACAGACAGGAG |
| | Mb-Cys | GATCATTAAGGCTTAGTTGTGCCGTATCCGGCAATATAGGTTCATC |
| | | CTATTGTATGGGATGGTTCCGTCAAGCACCCGGTAAGAAAAGGGAG |
| | | GCAGTGGCAAGAATAGTAAGGGACGGTGCAACCGGCTACGCAGATT |
| | | ACGTTAAGGGAAGGTTTAGGATTAGTAGAGATAGTGCAAAAAATAC |
| | | TCTATACCTTCAAATGAATAGGCTAATTCCCGAGGATACAGCTATA |
| | | TACTATTGCGCCGCAGACCTACCCCCAGGCTGTTTAACTCAGGCTA |
| | | TATGGAACTTCGGATATCGTGGCCAAGGAACCCTGGTCACGGTAAG |
| | | TTCAGAACCAAAATCCTCTGATAAAACCCATACTTGTCCACCTTGC |
| | | CCTGGTCAACCAAGAGAGCCCCAGGTATATACGCTGCCCCCTTCTC |
| | | GTGAAGAAATGACGAAAAATCAGGTGAGTCTAACGTGTTTAGTAAA |
| | | GGGATTTTACCCCTCCGATATAGCCGTTGAGTGGGAAAGTAATGGT |
| | | CAGCCAGAAAATAACTATAAAACGACACCCCCTGTGTTAGACTCAG |
| | | ACGGATCTTTCTTTTTATATTCAAAACTTACTGTAGATAAGTCCCG |
| | | TTGGCAGCAAGGTAATGTATTCTCATGTTCAGTGATGCATGAGGCC |
| | | CTGCATAACCATTATACCCAAAAGAGTCTATCCCTGTCTCCCGGCT |
| | | GC |
| 334 | HRasVHH | GAGGTCCAGCTTCTTGAATCCGGCGGAGGTCTGGTGCAGCCCGGTG |
| | Mb-Cys | GCTCTCTTCGTCTAAGTTGCGCCGCATCAGGCTTTACTTTCTCTAC |
| | | TTTTTCTATGAACTGGGTAAGACAAGCCCCAGGTAAGGGTCTAGAG |
| | | TGGGTTTCTTATATTTCTCGTACCTCTAAAACGATATATTATGCTG |
| | | ATTCAGTTAAGGGACGTTTTACGATATCTAGGGACAACAGTAAAAA |
| | | TACCTTGTATCTTCAAATGAACTCTCTGCGTGCAGAAGATACCGCA |
| | | GTGTATTACTGCGCTAGAGGCCGTTTTTTTGATTACTGGGGTCAAG |
| | | GAACACTTGTTACCGTATCTAGTGAGCCAAAGTCTTCCGACAAAAC |
| | | ACATACATGTCCTCCCTGCCCCGGACAGCCTCGTGAACCACAGGTA |
| | | TATACGCTTCCACCAAGTAGAGAGGAAATGACTAAGAATCAGGTTT |
| | | CACTGACATGCCTAGTGAAGGGCTTTTACCCTTCTGATATAGCTGT |
| | | GGAGTGGGAATCAAACGGCCAGCCCGAGAACAATTACAAAACGACT |
| | | CCACCTGTGCTAGACAGTGACGGCTCCTTTTTTTTATACTCAAAAC |
| | | TAACGGTAGACAAGTCTAGGTGGCAGCAGGGCAATGTTTTCAGTTG |
| | | TAGTGTCATGCACGAGGCACTACATAATCATTACACACAAAAGTCA |
| | | CTGTCTCTATCCCCCGGTTGT |
| 335 | Cys-BC2- | TGCCAGGTCCAACTGGTGGAGTCCGGCGGTGGTCTAGTCCAACCAG |
| | VHLpep | GAGGTTCATTGACCCTTTCATGTACCGCATCAGGTTTCACGCTTGA |
| | | CCACTACGACATCGGCTGGTTTCGTCAAGCCCCAGGCAAAGAGAGA |
| | | GAAGGTGTTTCCTGTATAAATAATTCCGATGACGACACATACTACG |
| | | CTGACTCCGTGAAGGGCAGGTTCACCATATTCATGAATAATGCTAA |
| | | AGATACCGTGTACTTGCAAATGAACAGTTTGAAGCCAGAAGACACA |
| | | GCCATCTATTATTGCGCCGAAGCCCGTGGTTGTAAACGTGGTAGGT |
| | | ATGAGTACGATTTCTGGGGTCAGGGTACACAGGTAACAGTCTCCTC |
| | | AGGAGGAGGCGGTTCATTGCCTGTCTACACATTGAAGGAAAGGTGC |
| | | CTACAAGTTGTCCGTAGTCTAGTTAAGCCTGAGAATTATAGGCGTC |
| | | TGGATATTGTGAGGTCCCTGTATGAAGACCTTGAGGACCATCCTAA |
| | | CGTC |
| 336 | VHLpep- | TTACCAGTCTATACTCTAAAAGAAAGGTGCCTACAAGTTGTTAGGT |
| | BC2-Cys | CCCTGGTAAAGCCTGAGAACTATAGGAGGTTAGACATCGTGCGTTC |
| | | CCTGTATGAGGACTTGGAGGATCACCCAAATGTGGGCGGTGGTGGC |
| | | TCACAAGTACAGCTAGTTGAAAGTGGTGGTGGACTTGTACAGCCTG |
| | | GTGGCTCCTTGACTTTATCCTGTACAGCATCAGGTTTTACGTTGGA |
| | | CCACTATGACATCGGATGGTTCCGTCAAGCCCCAGGAAAAGAGAGA |
| | | GAAGGCGTGTCATGCATCAACAATTCAGATGACGATACTTAGTATG |
| | | CTGACTCTGTGAAAGGAAGGTTTACTATTTTTATGAATAATGCTAA |
| | | AGATACGGTGTAGTTGCAGATGAACTCTCTTAAACCTGAAGATACA |
| | | GCAATTTATTATTGTGCTGAAGCAAGGGGATGCAAGAGAGGCAGGT |
| | | ACGAATACGATTTCTGGGGACAAGGCACCCAAGTTAGCGTTTCATC |
| | | CTGC |
| 337 | Cys-7D12- | TGCCAGGTCAAACTGGAAGAGTCAGGCGGCGGTTCTGTTCAAACTG |
| | VHLpep | GTGGAAGTCTTAGGTTAACGTGTGCCGCATCTGGCAGGACATCCAG |
| | | AAGTTATGGCATGGGATGGTTTCGTCAGGCTCCAGGAAAGGAAAGG |
| | | GAGTTTGTGAGTGGTATATCTTGGAGGGGCGATAGTACGGGATACG |
| | | CAGACTCAGTGAAGGGAAGATTTACCATATCCCGTGACAATGCTAA |
| | | GAATACCGTAGATCTGCAAATGAACTCCCTGAAGCCAGAGGATACA |
| | | GCCATATATTACTGTGCTGCAGCCGCCGGATCAGCTTGGTATGGAA |
| | | CACTATATGAGTATGATTACTGGGGCCAAGGAACCCAAGTGACTGT |
| | | TTCATCTGGTGGCGGAGGTTCCCTACCAGTATATACCCTGAAAGAA |
| | | AGATGTCTACAAGTCGTCAGATCCTTGGTCAAACCCGAGAATTATC |
| | | GTAGGTTGGATATTGTAAGAAGTCTATACGAGGACCTTGAAGACCA |
| | | CCCCAATGTT |
| 338 | VHLpep- | TTGCCTGTCTACACTCTGAAGGAGAGGTGCCTTCAGGTAGTCAGGT |
| | 7D12-Cys | CCCTTGTCAAACCCGAAAATTATCGTAGGCTGGATATAGTCCGTTC |
| | | CCTATATGAAGACCTAGAAGACCACCCCAATGTAGGTGGTGGAGGA |
| | | TCACAAGTGAAACTGGAAGAAAGTGGTGGAGGCAGTGTCCAAACAG |
| | | GAGGATCACTGCGTCTAACGTGCGCTGCTAGTGGCAGAACTTCACG |
| | | TTCCTATGGAATGGGCTGGTTCAGGCAAGCACCAGGCAAGGAGCGT |
| | | GAATTTGTCTCTGGAATCTCCTGGCGTGGCGATTCCACAGGCTATG |
| | | CAGACTCAGTAAAGGGCAGGTTTACAATTTCAAGGGACAACGCCAA |
| | | AAACACTGTCGATCTTCAGATGAACAGTCTAAAACCCGAAGACACA |
| | | GCAATATACTACTGCGCCGCCGCCGCCGGAAGTGCCTGGTATGGCA |
| | | CGCTTTATGAATATGATTACTGGGGTCAAGGAACCCAGGTCACTGT |
| | | TTCCAGTTGC |
| 339 | VHLpep- | TTGCGAGTATATACCCTGAAGGAAAGATGTCTTCAAGTAGTAAGAT |
| | ASCVHH | GAGTTGTTAAGCCCGAAAACTATAGAAGATTGGATATTGTACGTAG |
| | | TCTATATGAAGATTTGGAGGACCATCCAAATGTCGGTGGAGGTGGT |
| | | AGTCAGTTGCAGCTGGTGGAGTCAGGTGGTGGCCTGGTCCAACCAG |
| | | GCGGATCATTAAAATTATCCTGCGCTGCAAGTGGCTTTACGTTCAG |
| | | TAGATATGCTATGAGTTGGTACAGGCAAGCTCCAGGAAAGGAAAGA |
| | | GAATCTGTAGCACGTATATCTTCAGGCGGAGGCACGATCTATTATG |
| | | CCGACTCCGTTAAGGGCCGTTTTACCATATCCCGTGAAGACGCTAA |
| | | AAATACAGTATATCTGCAAATGAACTCTTTGAAGCCAGAGGATACT |
| | | GCCGTTTACTATTGCTACGTAGGCGGTTTCTGGGGCCAAGGTACTC |
| | | AGGTTACGGTGAGTTCCTGC |
| 340 | Cys- | TGTCAAGTCCAACTTCAAGAAAGTGGTGGTGGCCTGGTACAACCAG |
| | ASC^{VHH}_ | GAGGATCTTTAAAGCTGAGTTGTGCTGCATCCGGTTTTACTTTCTC |
| | VHL₁₁₀₋₂₁₃ | ACGTTACGCAATGAGTTGGTATAGACAGGCTCCCGGCAAGGAAAGG |
| | | GAGTCTGTGGCCAGGATATCAAGTGGAGGTGGCACCATCTACTACG |
| | | CTGACTCTGTGAAGGGAAGATTCACTATCTCAAGGGAGGACGCAAA |
| | | GAACACGGTTTACTTGCAAATGAATAGTCTGAAACCTGAAGACACA |
| | | GCCGTTTATTACTGTTATGTGGGCGGATTCTGGGGACAGGGAACGC |
| | | AAGTAACAGTTAGTTCCGGTGGCGGCGGCTCTCATAGTTATAGGGG |
| | | CCATTTATGGTTGTTCAGGGACGCAGGTACACATGACGGACTGCTA |
| | | GTAGCCCAGACAGAGCTATTTGTTCCATCCCTTAATGTGGATGGAC |
| | | AGCCAATCTTTGCCGCTATAACCTTACCTGTTTATACGTTGAAGGA |
| | | AAGATGTCTACAAGTGGTAAGATCTTTGGTAAAGCCCGAGAACTAT |
| | | AGAAGGCTGGACATTGTCCGTTCCCTGTACGAAGACTTGGAGGATC |
| | | ATCCCAACGTACAAAAAGACCTAGAAAGGCTAACCCAAGAGAGGAT |
| | | TGCCCATCAGAGAATGGGTGAT |
| 341 | Cys- | TGCCAGGTTCAGCTACAGGAGTCCGGCGGTGGCCTAGTTCAACCAG |
| | ASC^{VHH}_ | GTGGTTCATTGAAATTAAGTTGCGCCGCCAGTGGCTTTACCTTCTC |
| | VHL₁₃₈₋₂₁₃ | CAGGTACGCCATGTCATGGTATAGACAGGCACCTGGTAAGGAGAGA |
| | | GAATCAGTTGCTAGGATATCTTCAGGAGGCGGTACGATTTATTATG |
| | | CCGACTCAGTTAAGGGACGTTTCACAATATCCCGTGAGGACGCAAA |
| | | AAATACGGTCTATTTACAAATGAACTCCCTGAAGCCCGAGGATACA |
| | | GCCGTTTATTACTGCTACGTTGGAGGCTTCTGGGGACAAGGCACAC |
| | | AGGTAACGGTTAGTTCTGGCGGAGGAGGTTCACCCTCATTAAACGT |
| | | GGATGGTCAACCCATTTTCGCCGCTATCACCTTGCCTGTGTACACG |
| | | CTGAAGGAGAGATGCTTGCAGGTCGTGAGGAGTCTTGTTAAGCCTG |
| | | AAAACTATAGAAGGTTAGATATTGTCAGATCCCTATATGAAGACTT |
| | | AGAGGATCATCCCAACGTTCAAAAAGACCTGGAGAGACTAACCCAA |
| | | GAAAGAATAGCTCATCAACGTATGGGAGAT |
| 342 | Cys- | TGCCAAGTTCAGCTACAAGAATCAGGCGGAGGTCTTGTACAACCAG |
| | ASC^{VHH}_ | GAGGCTCTCTAAAACTTTCATGTGCCGCATCTGGTTTCACCTTCTC |
| | VHL₁₅₂₋₂₁₃ | CCGTTATGCAATGAGTTGGTACAGACAAGCTCCCGGAAAAGAGAGA |
| | | GAATCAGTGGCAAGAATTTCAAGTGGCGGTGGAACGATATATTATG |
| | | CCGATTCCGTAAAAGGCAGGTTCACCATATCAAGGGAGGACGCCAA |
| | | GAATACAGTATATCTGCAAATGAACTCCCTAAAACCCGAAGATACG |
| | | GCTGTGTACTATTGCTACGTGGGCGGTTTTTGGGGTCAAGGAACCC |
| | | AGGTTACCGTTAGTTCTGGTGGCGGTGGTAGTACGCTGCCCGTTTA |
| | | TACACTAAAAGAAAGATGCCTACAAGTAGTAGGTTCACTTGTTAAA |
| | | CCAGAGAACTACAGGCGTTTAGACATCGTTAGGAGTCTTTATGAAG |
| | | ACCTAGAAGATCATCCAAACGTCCAAAAAGATTTAGAAAGGCTGAC |
| | | ACAAGAACGTATAGCTCACCAGAGAATGGGTGAT |
| 343 | Cys-BC2- | TGTCAGGTACAGCTAGTGGAAAGTGGCGGCGGACTGGTTCAGCCTG |
| | SOCS1pep | GCGGTTCCTTGACACTATCCTGTACGGCTAGTGGATTCACTTTGGA |
| | | TCACTACGATATAGGCTGGTTCAGGCAAGCCCCTGGTAAGGAACGT |
| | | GAAGGCGTCTCTTGTATCAATAACTCAGACGATGATACATATTACG |
| | | CCGACTCAGTGAAAGGAAGGTTTAGAATCTTTATGAATAACGCCAA |
| | | GGATACAGTCTATCTTCAAATGAATAGTCTTAAGCCTGAGGACACC |
| | | GCTATATACTATTGCGCCGAGGCCAGAGGTTGCAAAAGGGGTAGAT |
| | | ATGAGTACGACTTTTGGGGTCAGGGAACCCAGGTTACGGTCTCCTC |
| | | TGGTGGTGGCGGTTCAGTCAGACCTCTGCAAGAATTATGCAGGCAA |
| | | AGAATCGTGGCCACTGTCGGTCGTGAAAATTTGGCCCGTATCCCTC |
| | | TAAACCCCGTCCTGCGTGACTACCTTTCATCATTCCCATTCCAAAT |
| | | T |
| 344 | SOCS1pep- | GTTCGTCCTTTACAGGAATTATGCAGGCAACGTATCGTCGCCACTG |
| | BC2-Cys | TGGGACGTGAGAATCTAGCCAGGATTCCCCTTAATCCAGTCTTGAG |
| | | GGATTATCTATCATCATTTCCATTCCAGATAGGTGGTGGAGGTTCC |
| | | CAGGTTCAATTGGTAGAAAGTGGTGGTGGACTTGTCCAACCAGGTG |
| | | GTTCCCTAACCCTAAGTTGCACTGCCTCTGGATTCACGCTGGACCA |
| | | TTACGACATCGGCTGGTTTAGACAAGCACCTGGAAAAGAGAGAGAA |
| | | GGCGTCTCCTGCATAAACAACTCCGATGACGATACATATTACGCTG |
| | | ATTCAGTCAAGGGTCGTTTCACTATCTTCATGAATAATGCCAAGGA |
| | | CACTGTCTACCTTCAAATGAATAGTTTGAAGCCTGAAGACACAGCC |
| | | ATTTATTATTGTGCAGAAGCAAGGGGCTGTAAGAGGGGCAGGTATG |
| | | AATATGATTTTTGGGGTCAGGGCACGCAGGTGACCGTTAGTAGTTG |
| | | C |
| 345 | Cys-7D12- | TGTCAAGTCAAGTTAGAGGAATCTGGCGGAGGATCAGTCCAGACGG |
| | SOCS1pep | GCGGCAGTTTGAGGCTTACCTGTGCAGCATCCGGTAGAACGTCAAG |
| | | GTCCTACGGTATGGGATGGTTTCGTCAGGCCCCAGGAAAGGAGAGG |
| | | GAGTTTGTGAGTGGTATATCATGGAGGGGCGACTCAACTGGCTATG |
| | | CAGATTCTGTAAAGGGTCGTTTTACAATCTCCAGAGATAACGCCAA |
| | | AAACACGGTCGACCTGCAGATGAACAGTCTGAAACCTGAAGACACA |
| | | GCTATCTACTATTGTGCCGCCGCAGCCGGATCTGCTTGGTATGGAA |
| | | CTTTATACGAATATGACTATTGGGGCCAAGGCACACAAGTCACCGT |
| | | TTCTAGTGGCGGTGGCGGAAGTGTGCGTCCCCTACAGGAACTTTGT |
| | | AGACAGAGAATCGTCGCTACCGTAGGCAGAGAAAACTTGGCTAGGA |
| | | TCCCTTTGAACCCAGTCTTACGTGACTATCTGTCATCCTTCCCATT |
| | | TCAAATC |
| 346 | SOCS1pep- | GTACGTCCATTGCAGGAGTTATGTCGTCAACGTATCGTGGCAACCG |
| | 7D12-Cys | TAGGCAGAGAGAACTTGGCCCGTATCCCTCTAAACCCCGTATTACG |
| | | TGATTATCTTTCATCTTTCCCTTTCCAGATCGGAGGCGGTGGATCT |
| | | CAGGTTAAGCTGGAAGAATCAGGAGGAGGAAGTGTACAGACTGGAG |
| | | GTTCTTTACGTTTAACGTGTGCTGCTAGTGGTCGTACATCACGTTC |
| | | ATACGGCATGGGTTGGTTCAGACAAGCCCCTGGCAAGGAAAGAGAA |
| | | TTTGTATCAGGTATCTCATGGAGAGGTGACAGTACGGGCTACGCCG |
| | | ACTCTGTAAAAGGCAGGTTTACGATTTCCAGGGATAACGCAAAGAA |
| | | TAGTGTTGACTTACAGATGAACTCTTTGAAACCTGAAGACACCGCT |
| | | ATCTATTATTGCGCAGCCGCTGCCGGTAGTGCATGGTATGGCACCC |
| | | TTTATGAATATGACTATTGGGGCCAGGGTACACAGGTAACGGTCAG |
| | | TTCCTGC |
| 347 | Cys- | TGCCAGCTTCAACTTGTCGAAAGTGGTGGAGGCCTTGTGCAACCAG |
| | ASC^{VHH}_ | GAGGTAGTCTAAAATTGTCATGTGCTGCATCAGGCTTCACATTCTC |
| | SOCS1pep | TAGATACGCCATGTCTTGGTATCGTCAAGCTCCTGGTAAGGAACGT |
| | | GAATCCGTAGCTCGTATCTCTTCAGGTGGTGGCACCATTTACTACG |
| | | CCGATTCTGTGAAAGGCAGATTTACGATTTCTCGTGAGGATGCCAA |
| | | GAACACGGTCTACCTTCAGATGAACTCCCTAAAACCTGAAGACACG |
| | | GCAGTCTACTATTGTTACGTGGGTGGCTTTTGGGGTCAAGGTACTC |
| | | AGGTTACAGTATCATCAGGCGGCGGCGGATCTGTGAGACCACTACA |
| | | GGAGTTATGTCGTCAGCGTATAGTCGCTACCGTAGGACGTGAAAAT |
| | | TTAGCTCGTATTCCCCTGAACCCCGTCCTACGTGATTACCTATCCT |
| | | CATTTCCCTTCCAAATA |
| 348 | SOCS1pep- | GTCCGTCCTCTTCAAGAGTTATGCAGGCAACGTATCGTTGCTACAG |
| | -ASC^{VHH}_ | TAGGAAGAGAGAACTTAGCCCGTATCCCACTGAACCCTGTCTTGAG |
| | Cys | GGACTATCTTTCTTCTTTCCCATTCCAGATAGGAGGTGGCGGATCA |
| | | CAGCTACAACTTGTCGAGTCTGGTGGTGGCCTAGTACAACCAGGTG |
| | | GCTCCCTGAAACTGTCCTGCGCTGCCAGTGGTTTTACTTTTAGTCG |
| | | TTATGCTATGAGTTGGTACAGGCAGGCACCCGGCAAGGAAAGAGAG |
| | | TCAGTCGCTAGGATTTCCTCTGGCGGTGGTACGATATATTATGCCG |
| | | ATAGTGTCAAGGGTAGGTTCACCATAAGTCGTGAGGACGCTAAAAA |
| | | CACAGTGTATCTTCAAATGAACTCACTAAAACCAGAGGACACAGCT |
| | | GTTTATTACTGCTACGTAGGTGGCTTTTGGGGTCAAGGTACACAAG |
| | | TCACGGTCAGTTCCTGT |
| 349 | K19-hFc- | GACTTGGGTAAAAAGCTGCTTGAGGCAGCTAGAGCAGGACAAGATG |
| | Cys | ACGAGGTAAGAATACTAATGGCAAATGGAGCAGATGTCAATGCCTC |
| | | CGATAGATGGGGATGGACCCCATTACATCTGGCAGCATGGTGGGGA |
| | | CACCTTGAAATTGTCGAAGTGCTTTTAAAACGTGGCGCTGATGTCT |
| | | CTGCTGCTGATTTGCACGGCCAGAGTCCTCTACACCTAGCTGCTAT |
| | | GGTCGGCCATCTAGAGATTGTGGAAGTATTACTGAAGTATGGTGCC |
| | | GACGTAAATGCCAAAGACACCATGGGAGCAACACCTCTTCACCTGG |
| | | CAGCTAGAAGTGGCCACTTAGAAATCGTAGAAGAGTTGCTGAAGAA |
| | | TGGTGCCGACATGAATGCCCAGGATAAGTTCGGAAAGACTACTTTC |
| | | GACATCTCTACAGATAACGGCAACGAGGACTTAGCAGAGATTCTAG |
| | | AAAAACTTGGTGGAGGCGGATCAGACAAGACGCACACGTGTCCTCC |
| | | CTGTCCTGCTCCTGAATTGTTGGGCGGACCATCCGTTTTTCTTTTT |
| | | CCCCCCAAGCCTAAAGACACTCTTATGATCTCTCGTACCCCTGAAG |
| | | TTACTTGCGTAGTTGTCGATGTATCACATGAGGACCCTGAGGTGAA |
| | | ATTTAACTGGTATGTTGACGGAGTAGAGGTTCACAACGCTAAGACA |
| | | AAACCACGTGAGGAACAATATGCCAGTAGATACCGTGTCGTATCTG |
| | | TGCTAACTGTACTACATCAAGACTGGCTTAATGGCAAAGAATATAA |
| | | ATGCAAGGTGTCAAACAAGGCACTTCCAGCCCCCATTGAAAAAACT |
| | | ATTAGTAAAGCCAAGGGTCAACCTCGTGAGCCCCAAGTCTACACAC |
| | | TACCACCTTCACGTGAAGAGATGACAAAAAACCAGGTATCCTTAAC |
| | | CTGTTTGGTCAAAGGCTTCTATCCTTCAGATATTGCAGTTGAGTGG |
| | | GAATCTAACGGCCAGCCTGAGAATAACTACAAAACTACACCCCCTG |
| | | TCTTGGATTCCGACGGTAGTTTCTTCCTATACAGTAAACTAACTGT |
| | | AGATAAGAGTAGGTGGCAGCAGGGCAATGTCTTCTCCTGCTCCGTG |
| | | ATGCACGAAGCTCTACACAATCATTATACCCAAAAGTCATTGTCAT |
| | | TGTCACCCGGTTGC |
| 350 | K19-hFc- | GATTTGGGTAAGAAATTGTTAGAGGCCGCAAGGGCAGGACAAGATG |
| | Cys | ATGAGGTTCGTATCTTAATGGCAAATGGTGCCGACGTGAATGCATC |
| | | CGACAGATGGGGCTGGACTCCTCTGCATTTAGCCGCTTGGTGGGGC |
| | | CACCTGGAAATAGTTGAAGTATTGTTGAAGAGAGGTGCTGATGTTT |
| | | CAGCCGCCGATTTGCATGGACAGTCCCCCTTACATCTTGCTGCAAT |
| | | GGTCGGTCATCTAGAAATCGTAGAGGTTCTATTGAAATACGGAGCA |
| | | GATGTCAACGCCAAGGATACGATGGGAGCTACTCCTCTACACTTGG |
| | | CCGCAAGAAGTGGCCATTTGGAAATAGTCGAGGAGTTATTGAAGAA |
| | | TGGAGCAGATATGAATGCCCAGGACAAGTTCGGAAAAACAACTTTC |
| | | GACATATCTACAGATAACGGAAATGAGGATTTGGCTGAAATACTGC |
| | | AAAAACTAGGTGGAGGCGGCTCAGATAAAACTCACACATGTCCCCC |
| | | TTGCCCCGCCCCTGAGTTACTTGGAGGACCATCTGTTTTCCTGTTT |
| | | CCTCCCAAACCAAAAGACACCCTTATGATTTCTAGGACACCCGAGG |
| | | TAACATGTGTGGTTGTTGACGTTTCACATGAAGACCCCGAGGTGAA |
| | | ATTCAATTGGTATGTAGACGGCGTAGAGGTCCATAATGCCAAAACA |
| | | AAACCTCGTGAAGAGCAATACGCCTCCACATATAGAGTTGTATCTG |
| | | TGCTAACCGTCTTACATCAAGATTGGCTAAATGGTAAAGAGTATAA |
| | | GTGCAAAGTGTCCAACAAGGCCCTACCTGCCCCAATTGAAAAAACG |
| | | ATATCTAAAGCAAAAGGTCAGCCTAGAGAACCACAGGTCTATACAT |
| | | TACCTCCTTCCAGAGAAGAAATGACGAAGAACCAGGTAAGTTTAAC |
| | | TTGTCTAGTAAAAGGTTTTTACCCATCTGACATTGCTGTAGAATGG |
| | | GAAAGTAATGGTCAACCTGAGAATAATTACAAAACGACACCACCTG |
| | | TTCTGGATTCCGATGGCTCCTTTTTTTTGTATTCCAAGTTAACTGT |
| | | TGATAAATCACGTTGGCAACAAGGCAATGTCTTTTCCTGTTCAGTT |
| | | ATGCACGAGGCACTTGCTAATCACTACACACAGAAGTCTCTATCTC |
| | | TGTCCCCTGGTTGT |
| 351 | K19-hFc | GACCTAGGCAAAAAGTTATTGGAGGCCGCAAGAGCTGGCCAAGATG |
| | | ACGAAGTGCGTATTTTAATGGCCAACGGAGCTGACGTCAACGCCAG |
| | | TGATAGGTGGGGTTGGACCCCCCTTCATCTTGCCGCCTGGTGGGGC |
| | | CACCTGGAGATCGTCGAAGTACTTTTAAAGAGAGGAGCTGATGTCA |
| | | GTGCTGCTGATTTGCATGGCCAATCCCCTCTTCATTTGGCCGCAAT |
| | | GGTGGGACACTTAGAAATAGTAGAAGTCTTAGTTAAATATGGAGCA |
| | | GACGTCAATGCAAAGGACACGATGGGAGCTACACCATTGCACTTAG |
| | | GAGCACGTTGAGGAGATCTTGAGATTGTTGAAGAACTGCTGAAAAA |
| | | CGGTGCAGACATGAATGCCCAGGATAAGTTCGGTAAGACTACATTC |
| | | GACATTTCTACAGACAACGGAAATGAGGATCTGGCCGAGATACTTC |
| | | AAAAACTGGGCGGAGGTGGTAGTGGCGGTGGCGGTTCCGAGCCCAA |
| | | GTCATCAGACAAAACACACACTTGTCCACCCTGTCCAGCCCCAGAG |
| | | TTACTTGGAGGCCCTTGCGTTTTCTTATTCCCACCAAAGCCCAAAG |
| | | ATACCCTGATGATAAGTAGAACCCCCGAAGTCACATGCGTAGTTGT |
| | | TGATGTGTCACACGAGGACCCCGAAGTGAAGTTCAACTGGTACGTC |
| | | GACGGTGTCGAGGTGCATAATGCTAAAACTAAGCCCCGTGAAGAGC |
| | | AATACGCTTCCACGTATAGGGTCGTTTCTGTGCTTACAGTGTTACA |
| | | CCAAGATTGGCTGAATGGAAAGGAGTATAAATGCAAAGTCTCAAAC |
| | | AAGGCTCTTCCCGCCCCAATCGAGAAAACTATATCTAAAGCAAAAG |
| | | GTCAACCTCGTGAGCCTCAAGTCTACACTCTACCTCCTTCTAGAGA |
| | | CGAGCTGACTAAGAACCAGGTGTCACTGACGTGCCTTGTGAAAGGA |
| | | TTCTATCCCAGTGATATCGCAGTAGAGTGGGAGTCTAATGGACAAC |
| | | CCGAAAATAACTATAAGACCACACCCCCAGTACTGGACTCAGACGG |
| | | CAGTTTCTTCTTATACAGTAAGTTGACCGTTGACAAATCTCGTTGG |
| | | CAGCAAGGAAACGTCTTCTCTTGCAGTGTCATGCATGAGGCTCTGC |
| | | ATAACCACTACACGCAAAAGTCCTTATCTTTGTCCCCCGGCAAA |
| 352 | hFc-K19 | GATAAGACCCACACGTGCCCACCCTGCCCTGCCCCCGAACTTCTTG |
| | | GTGGACCTTGTGTATTTCTATTCCCTCCCAAGCCCAAGGATACTTT |
| | | AATGATTTCCAGGACCCCTGAGGTGACATGCGTTGTAGTTGACGTA |
| | | TCACATGAAGACCCAGAAGTCAAATTTAACTGGTATGTAGACGGCG |
| | | TGGAGGTGCATAACGCCAAAACAAAGCCTAGGGAGGAACAATATGC |
| | | ATCAACGTACAGGGTCGTCTCCGTACTAACGGTGTTACATCAAGAC |
| | | TGGCTAAATGGTAAGGAATACAAATGTAAAGTGAGTAACAAGGCCC |
| | | TACCCGCCCCTATCGAGAAAACAATCTCCAAGGCAAAGGGTCAACC |
| | | TAGAGAACCCCAGGTATATACGCTTCCCCCTTCCAGGGATGAGTTA |
| | | ACCAAGAATCAAGTAAGTTTGACATGCTTGGTTAAGGGCTTCTACC |
| | | CCTCCGATATTGCCGTGGAATGGGAGTCCAATGGCCAGCCCGAGAA |
| | | TAATTATAAGACTACCCCTCCAGTCCTGGATTCAGACGGCTCATTT |
| | | TTTCTTTACTCAAAGCTAACGGTGGACAAGAGTAGATGGCAGCAGG |
| | | GAAATGTCTTTTCATGCTCAGTAATGCATGAAGCATTAGATAACCA |
| | | CTATACGCAAAAATCCCTTTCTCTTAGTCCCGGTGGAGGTGGAGGA |
| | | TCAGGAGGCGGCGGCTCCGATTTGGGTAAAAAATTGTTAGAGGCTG |
| | | CCAGAGCCGGACAGGATGATGAAGTCAGGATCTTGATGGCAAATGG |
| | | TGCAGACGTGAATGCTTCAGATAGATGGGGATGGACCCCACTACAT |
| | | CTAGCCGCATGGTGGGGCCACCTGGAAATTGTTGAAGTACTTTTAA |
| | | AGCGTGGTGCCGACGTGTCAGCTGCAGATCTACACGGTCAATCACC |
| | | CTTGCACCTTGCAGCCATGGTGGGTCATTTAGAGATTGTCGAAGTT |
| | | CTTCTTAAATACGGAGCTGACGTAAATGCTAAGGACACTATGGGTG |
| | | CTACTCCCTTACATCTAGCTGCAAGGTCCGGCCATTTGGAGATCGT |
| | | CGAGGAGCTATTAAAGAACGGTGCTGACATGAATGCACAGGATAAA |
| | | TTTGGAAAAACGACGTTTGATATATCCACTGACAACGGCAATGAAG |
| | | ACTTAGCTGAGATCCTGCAAAAGTTA |
| 353 | K19-Mb- | GACCTAGGAAAAAAGTTGCTTGAGGCCGCTAGGGCCGGACAAGATG |
| | Cys | ATGAAGTGCGTATCTTAATGGCTAACGGTGCCGATGTCAATGCCAG |
| | | TGACAGGTGGGGATGGACTCCTTTGCACTTGGCAGCATGGTGGGGT |
| | | CATTTGGAAATTGTAGAGGTGCTTTTGAAGAGGGGTGCCGACGTTT |
| | | CTGCTGCTGATCTTCACGGTCAGAGTCCCCTTCACCTTGCTGCTAT |
| | | GGTTGGTCATTTAGAAATTGTGGAAGTTCTATTAAAGTACGGCGCA |
| | | GATGTCAATGCTAAAGACACAATGGGTGCTAGTCCATTGCATTTAG |
| | | CTGCTAGGTCTGGACACTTAGAAATAGTAGAAGAGTTGCTTAAGAA |
| | | TGGCGCTGACATGAACGCCCAAGACAAGTTTGGAAAAACCACGTTT |
| | | GACATTAGTACGGATAATGGCAACGAAGACTTGGCAGAAATCTTAC |
| | | AGAAGCTGGGAGGAGGTGGTAGTGAACCTAAATCTTCAGATAAGAC |
| | | CCATACTTGCCCCCCATGTCCTGGACAACCCAGGGAGCCTCAAGTG |
| | | TATACTTTGCCCCCTTCTAGGGAAGAGATGACCAAAAACCAAGTCT |
| | | CCTTGACGTGTTTGGTAAAAGGCTTTTACCCTTCCGATATCGCCGT |
| | | TGAATGGGAGAGTAACGGACAACCCGAAAACAATTACAAGACGACC |
| | | CCACCAGTGCTTGACTCTGATGGTAGTTTTTTTTTGTACAGTAAAC |
| | | TGACGGTTGATAAGTCTCGTTGGCAACAGGGCAATGTTTTTTCTTG |
| | | TTCAGTTATGCATGAAGCCTTGCATAACCACTATACGCAAAAATCA |
| | | CTGTCTCTTTCCCCCGGCTGT |
| 354 | Cys-K19- | TGTGACCTTGGTAAGAAACTACTAGAGGCAGCCAGGGCTGGCCAGG |
| | 7D12 | ATGACGAAGTGCGTATTTTGATGGCAAATGGAGCTGATGTAAACGC |
| | | CTCTGATAGATGGGGATGGACACCCCTTCATCTTGCTGCCTGGTGG |
| | | GGACACCTTGAGATCGTCGAAGTGTTATTGAAAAGGGGAGCAGATG |
| | | TGAGTGCCGCCGATCTGCATGGCCAAAGTCCATTGCATTTAGCCGC |
| | | AATGGTAGGACATTTAGAGATAGTGGAGGTACTTCTGAAGTATGGA |
| | | GCAGATGTTAATGCCAAAGACACAATGGGTGCTACCCCCCTTCATT |
| | | TGGCTGCAAGATCAGGCCATCTAGAAATAGTTGAGGAGTTATTAAA |
| | | GAATGGTGCCGATATGAATGCACAGGACAAGTTTGGTAAAACTACT |
| | | TTTGATATCTCAACTGATAATGGTAACGAGGATTTGGCAGAGATAT |
| | | TACAAAAGCTAGGAGGTGGAGGATCTGGAGGAGGCGGTTCTCAGGT |
| | | TAAGCTAGAGGAGAGTGGCGGAGGTTCCGTACAAACTGGAGGCTCA |
| | | TTGCGTTTGACTTGTGCAGCATCAGGTAGGACATCCCGTTCATACG |
| | | GAATGGGCTGGTTTAGACAGGCCCCCGGTAAAGAACGTGAGTTCGT |
| | | TTCTGGTATATCATGGAGAGGCGACTCTACCGGATATGCTGATTCA |
| | | GTCAAGGGTAGATTTACCATCTCACGTGATAATGCTAAAAACACGG |
| | | TTGACTTGCAAATGAACTCATTGAAACCTGAGGATACAGCAATATA |
| | | CTACTGCGCAGCCGCAGCCGGATCAGCATGGTATGGTACCCTGTAC |
| | | GAATACGATTACTGGGGACAGGGCACCCAGGTCACTGTATCCTCT |
| 355 | K19-7D12- | GACCTTGGCAAGAAGTTGCTAGAAGCTGCAAGAGCCGGCCAGGACG |
| | Cys | ACGAGGTTAGAATACTTATGGCCAACGGTGCAGATGTTAATGCTAG |
| | | TGATAGATGGGGCTGGACACCTTTACATCTAGCTGCTTGGTGGGGA |
| | | CATCTTGAAATCGTCGAGGTGTTGCTAAAGAGGGGAGCTGATGTGT |
| | | CAGCCGCAGACCTGCATGGTCAATCTCCACTACACCTGGCAGCTAT |
| | | GGTAGGTCACTTAGAGATAGTCGAGGTTCTGTTGAAATACGGCGCA |
| | | GATGTAAATGCTAAAGATACGATGGGTGCCACACCTTTACACTTAG |
| | | CAGCTAGGTCCGGTCACCTGGAAATAGTAGAAGAACTGCTAAAGAA |
| | | CGGAGCTGATATGAATGCTCAGGACAAATTTGGAAAGACTACTTTT |
| | | GATATTAGTACAGACAACGGTAATGAAGACCTAGCAGAAATCTTGC |
| | | AAAAACTGGGCGGCGGTGGTAGTGGAGGCGGAGGTTCACAGGTTAA |
| | | GTTGGAGGAATCTGGCGGCGGTTCCGTCCAGACCGGCGGCAGTTTA |
| | | AGATTGACATGTGCAGCATCTGGTCGTACTTCCAGAAGTTATGGCA |
| | | TGGGCTGGTTCAGGCAGGCTCCTGGTAAGGAAAGAGAGTTTGTGTC |
| | | TGGTATCTCATGGAGGGGCGACTCCACCGGCTATGCTGACTCAGTG |
| | | AAAGGCCGTTTCACAATCTCTAGAGATAACGCAAAGAATACCGTCG |
| | | ACCTGCAGATGAACAGTTTGAAGCCCGAGGATACGGCTATATACTA |
| | | CTGCGCCGCCGCAGCCGGTTCCGCTTGGTACGGAACCTTGTACGAA |
| | | TATGATTACTGGGGCCAAGGCACACAGGTCACAGTCTCCTCCTGC |
| 356 | Cys-7D12- | TGCCAGGTGAAATTAGAGGAATCTGGAGGTGGCTCCGTGCAGACAG |
| | K19 | GTGGAAGTCTGCGTCTTACTTGTGCTGCATCAGGACGTACGAGTAG |
| | | AAGTTATGGAATGGGATGGTTTAGACAGGCTCCCGGTAAGGAGAGA |
| | | GAGTTTGTCAGTGGAATTTCCTGGAGGGGCGATTCCACGGGATACG |
| | | CTGATTCAGTTAAAGGAAGATTCACCATCTCTCGTGACAACGCAAA |
| | | AAACACAGTGGATTTACAGATGAATAGTCTGAAGCCTGAGGATACC |
| | | GCCATATATTACTGCGCCGCAGCAGCAGGAAGTGCTTGGTACGGAA |
| | | CATTATACGAGTACGACTACTGGGGCCAAGGTACGCAAGTTACTGT |
| | | GTCTTCTGGTGGAGGCGGTTCCGGTGGAGGTGGCAGTGACTTAGGC |
| | | AAAAAGTTATTGGAGGCCGCAAGAGCCGGTCAAGACGACGAAGTGA |
| | | GGATACTTATGGCAAACGGTGCCGACGTGAATGCATCCGACAGGTG |
| | | GGGCTGGACCCCTCTTCACCTAGCCGCTTGGTGGGGACATCTTGAA |
| | | ATCGTAGAGGTGCTATTAAAGCGTGGAGCCGACGTCTCAGCAGCAG |
| | | ATTTACACGGACAGTCTCCTCTACATCTAGCAGCTATGGTGGGTCA |
| | | CTTGGAAATTGTGGAGGTGTTACTAAAGTATGGTGCAGATGTAAAC |
| | | GCCAAAGATACAATGGGCGCAACGCCATTGCATCTTGCCGCTAGAT |
| | | CCGGCCATTTGGAGATAGTCGAAGAGCTATTGAAAAACGGCGCTGA |
| | | TATGAACGCCCAAGACAAGTTCGGAAAAACTACATTTGATATATCT |
| | | ACAGACAACGGTAACGAAGACTTGGCCGAGATTCTTCAGAAGTTA |
| 357 | 7D12- | CAGGTTAAGCTTGAAGAATCCGGCGGTGGATCAGTTCAAACTGGCG |
| | linker-K19- | GAAGTTTGCGTCTTACTTGTGCAGCTTCAGGCAGAACCAGTCGTAG |
| | Cys | TTACGGAATGGGTTGGTTCCGTCAGGCACCTGGAAAAGAAAGAGAG |
| | | TTCGTGAGTGGAATTTCATGGCGTGGCGATTCTACAGGCTACGCTG |
| | | ACAGTGTAAAGGGACGTTTCACGATCTCACGTGATAACGCAAAAAA |
| | | TAGAGTTGACTTGCAAATGAACTCCTTAAAGCCAGAGGACACCGCT |
| | | ATATATTATTGTGCCGCAGCAGCAGGTTCTGCATGGTATGGAACCT |
| | | TATACGAATATGACTATTGGGGTCAAGGAACCCAAGTTACTGTCAG |
| | | TTCTGGTGGAGGCGGCAGTGGAGGTGGAGGCTCCGACTTGGGAAAA |
| | | AAACTGTTGGAGGCAGCAAGAGCAGGACAAGATGACGAGGTTCGTA |
| | | TACTTATGGCTAATGGAGCCGACGTGAACGCAAGTGATAGGTGGGG |
| | | TTGGACACCCCTACACCTAGCTGCCTGGTGGGGACACCTAGAAATC |
| | | GTGGAAGTTCTACTAAAAAGGGGCGCCGACGTTTCAGCCGCAGACC |
| | | TACACGGTCAATCCCCATTGCATCTAGCAGCTATGGTAGGACATCT |
| | | TGAGATTGTAGAAGTGCTACTAAAGTACGGTGCCGATGTAAATGCC |
| | | AAAGACACTATGGGAGCAACGCCATTACATCTAGCCGCAAGGTCAG |
| | | GTCATTTAGAAATTGTCGAGGAGTTACTAAAAAATGGCGCTGATAT |
| | | GAACGCTCAAGATAAATTCGGTAAAACGACTTTTGACATATCAACA |
| | | GATAATGGAAACGAAGACCTTGCCGAAATTTTGCAGAAGCTTTGC |
| 358 | Cys-7D12- | TGTCAGGTGAAATTAGAAGAATCCGGCGGTGGTAGTGTGCAGACCG |
| | K19- | GCGGAAGTCTACGTCTTACCTGCGCTGCCAGTGGTAGGACATCCCG |
| | VHL152-213 | TTCCTACGGTATGGGCTGGTTTCGTCAGGCTCCAGGAAAAGAAAGA |
| | | GAGTTTGTAAGTGGTATATCCTGGCGTGGCGATTCCACCGGATACG |
| | | CCGACTCAGTCAAAGGCCGTTTCACAATCTCCAGAGATAACGCCAA |
| | | AAATACGGTTGACCTACAGATGAACAGTCTAAAGCCCGAAGACACG |
| | | GCAATTTACTATTGTGCAGCCGCCGCCGGATCCGCCTGGTATGGAA |
| | | CTCTGTATGAGTACGACTACTGGGGACAAGGCACCCAGGTGACAGT |
| | | GAGTTCTGGAGGAGGCGGCAGTGGTGGTGGAGGATCAGACCTGGGA |
| | | AAGAAGTTACTGGAAGCAGCTAGAGCCGGACAGGACGATGAAGTCA |
| | | GGATATTGATGGCTAACGGTGCTGACGTCAACGCAAGTGACAGATG |
| | | GGGCTGGACGCCTTTGCATCTAGCTGCATGGTGGGGCCATTTGGAG |
| | | ATAGTGGAGGTACTTCTTAAAAGAGGTGCCGACGTGTCCGCTGCTG |
| | | ATCTTCATGGCCAATCTCCACTACATTTAGCAGCAATGGTCGGACA |
| | | TTTGGAAATCGTCGAAGTCTTGCTGAAGTATGGAGCAGATGTCAAT |
| | | GCAAAAGACACAATGGGAGCTACTCCCCTTCACTTAGCCGCCAGGA |
| | | GTGGCCACTTAGAGATTGTGGAGGAACTACTTAAAAATGGTGCCGA |
| | | TATGAACGCCCAAGACAAGTTCGGTAAGACGACCTTCGACATCTCT |
| | | ACCGATAACGGTAATGAAGACCTTGCAGAAATACTGCAGAAACTAG |
| | | GAGGTGGAGGTAGTACTTTGCCTGTATATACGTTAAAAGAGAGGTG |
| | | CTTGCAGGTAGTACGTTCTCTAGTGAAGCCAGAAAATTACAGGCGT |
| | | TTGGATATCGTTAGATCCCTGTATGAGGATCTGGAGGATCACCCCA |
| | | ATGTGCAGAAGGACTTGGAGAGATTAACCCAAGAGAGGATCGCACA |
| | | TCAAAGGATGGGCGAT |
| 359 | Cys-7D12- | TGTCAGGTTAAGTTGGAAGAATCAGGCGGTGGCAGTGTCCAGACGG |
| | K19- | GTGGATCACTGAGGCTTACGTGCGCAGCATCCGGAAGAACGAGTAG |
| | VHLpep | GTCTTACGGAATGGGATGGTTTAGGCAAGCTCCTGGTAAGGAGCGT |
| | | GAGTTCGTATCAGGTATATCATGGCGTGGAGATTCTACAGGCTATG |
| | | CAGACTCTGTCAAAGGCAGGTTCACTATATCTAGGGACAACGCTAA |
| | | AAATACGGTCGACCTACAAATGAATAGTCTTAAGCCCGAGGACACT |
| | | GCAATTTACTACTGCGCTGCCGCTGCAGGCTCTGCATGGTATGGTA |
| | | CTCTGTACGAGTACGATTACTGGGGCCAAGGAACTCAAGTCACAGT |
| | | ATCTTCAGGAGGTGGAGGTTCTGGAGGAGGCGGCTCAGATCTGGGC |
| | | AAGAAACTGTTAGAAGCCGCAAGAGCTGGACAAGACGACGAAGTAA |
| | | GAATCCTAATGGCAAACGGAGCCGACGTAAATGCATCCGATCGTTG |
| | | GGGCTGGACACCATTACACTTGGCTGCTTGGTGGGGACACTTAGAG |
| | | ATTGTCGAGGTATTACTGAAGAGGGGAGCAGATGTATCCGCAGCAG |
| | | ATCTGCACGGTCAATCCCCACTACACTTAGCCGCAATGGTGGGCCA |
| | | TCTAGAAATTGTGGAAGTGCTTTTGAAATATGGTGCTGATGTTAAT |
| | | GCCAAGGATACGATGGGCGCCACGCCACTACATCTAGCCGCAAGGT |
| | | CAGGTCACCTGGAAATCGTCGAAGAACTGTTAAAAAACGGCGCTGA |
| | | TATGAATGCCCAGGATAAGTTCGGTAAGACCACCTTCGACATAAGT |
| | | ACTGATAATGGTAACGAAGACCTGGCTGAAATTTTGCAAAAGTTGG |
| | | GAGGTGGTGGCAGTTTGCCCGTTTATACACTGAAAGAAAGGTGTTT |
| | | ACAGGTGGTAAGATCCTTGGTGAAGCCAGAGAATTAGCGTAGATTG |
| | | GATATAGTTAGGTCTCTGTATGAGGACTTGGAAGATCACCCTAACG |
| | | TG |
| 360 | Cys-K19- | TGTGATCTGGGAAAAAAGCTTCTTGAAGCTGCCAGAGCTGGCCAAG |
| | 7D12- | ATGATGAGGTGCGTATTCTTATGGCAAACGGAGCTGATGTCAATGC |
| | VHL₁₅₂₋₂₁₃ | CAGTGACAGGTGGGGATGGACGCCTCTGCACTTGGCAGCATGGTGG |
| | | GGTCACCTAGAGATAGTAGAGGTTCTTCTAAAGAGGGGTGCAGATG |
| | | TTTCTGCTGCCGACTTACATGGACAGTCACCTCTTCACCTGGCAGC |
| | | CATGGTTGGCCATTTGGAGATAGTTGAGGTCCTATTAAAATACGGC |
| | | GCCGATGTGAATGCTAAAGATACCATGGGCGCCACGCCATTGCATC |
| | | TTGCCGCCAGATCTGGCCATCTTGAAATCGTCGAAGAGTTATTAAA |
| | | GAATGGCGCAGATATGAACGCACAGGATAAATTCGGCAAGACAACA |
| | | TTCGACATCTCAACAGACAACGGCAATGAAGACCTTGCTGAGATTC |
| | | TTCAAAAACTGGGAGGTGGTGGTTCCGGTGGAGGCGGTTCCCAAGT |
| | | GAAGTTAGAAGAGTCCGGAGGAGGATCAGTCCAAACGGGCGGTAGT |
| | | CTTAGGCTGACCTGCGCAGCATCCGGACGTACATCTCGTTCTTATG |
| | | GAATGGGCTGGTTCAGACAAGCTCCCGGTAAAGAAAGAGAGTTTGT |
| | | TTCTGGTATTTCTTGGAGAGGTGATTCAACAGGCTATGCTGACTCT |
| | | GTAAAAGGCAGATTCACTATCTCACGTGATAATGCAAAGAACACAG |
| | | TGGATCTTCAAATGAACTCCCTAAAACCAGAAGATACGGCTATTTA |
| | | TTACTGTGCAGCCGCCGCTGGCTCCGCTTGGTATGGAACGCTTTAC |
| | | GAATACGATTATTGGGGTCAGGGTACTCAGGTGACCGTTTCCAGTG |
| | | GAGGAGGAGGATCCACATTACCTGTTTACACCTTGAAGGAGCGTTG |
| | | CTTGCAAGTGGTTAGATCATTAGTGAAACCTGAAAATTACAGAAGG |
| | | CTAGATATTGTTAGATCCCTTTATGAGGACCTAGAGGATCATCCAA |
| | | ATGTGCAGAAAGACTTAGAAAGACTGACGCAAGAGAGGATTGCCCA |
| | | CCAGAGGATGGGAGAC |
| 361 | Cys-K19- | TGCGATCTGGGTAAAAAACTTTTAGAGGCAGCCAGGGCAGGCCAAG |
| | 7D12- | ACGATGAGGTTAGAATCTTGATGGCTAATGGTGCCGACGTGAACGC |
| | VHLpep | ATCCGATAGATGGGGTTGGACACCATTACACCTTGCAGCTTGGTGG |
| | | GGCCATCTAGAGATCGTAGAGGTGTTGCTAAAACGTGGTGCCGATG |
| | | TATCAGCAGCTGATCTGCATGGTCAGTCTCCTCTGCACCTTGCTGC |
| | | TATGGTGGGACATTTGGAGATCGTGGAGGTGCTATTGAAATACGGT |
| | | GCCGACGTTAATGCAAAGGATACAATGGGAGCTACGCCCCTGCATC |
| | | TAGGAGCTCGTTGAGGTGAGTTAGAAATAGTTGAAGAATTACTGAA |
| | | GAACGGAGCTGATATGAATGCACAAGATAAGTTTGGTAAAACGACG |
| | | TTCGATATAAGTACGGATAACGGCAACGAGGATCTGGCAGAGATTC |
| | | TACAAAAATTGGGCGGAGGCGGATCTGGTGGTGGCGGATCACAGGT |
| | | GAAGTTAGAGGAATCTGGAGGAGGCTCTGTTCAGACGGGCGGCTCT |
| | | CTGCGTTTGACTTGCGCTGCATCTGGTAGAACATCAAGGTCATACG |
| | | GCATGGGTTGGTTCCGTCAGGCCCCAGGAAAAGAGAGGGAGTTCGT |
| | | GAGTGGTATATCCTGGCGTGGAGATTCCACAGGTTATGCAGATAGT |
| | | GTAAAGGGTCGTTTCACGATAAGTCGTGACAACGCAAAAAACACCG |
| | | TGGACTTACAGATGAACAGTTTGAAACCAGAAGATACCGCTATTTA |
| | | CTATTGCGCAGCAGCCGCAGGTTCCGCATGGTACGGAACATTATAT |
| | | GAGTATGATTATTGGGGACAAGGCACCCAGGTTACCGTTTCTAGTG |
| | | GAGGAGGAGGATCATTACCCGTGTATACGCTTAAAGAGAGGTGTCT |
| | | TCAAGTGGTACGTTCTTTAGTCAAACCCGAAAATTACAGGCGTCTT |
| | | GATATTGTAGGTAGTCTATACGAGGATCTAGAAGATCATCCAAACG |
| | | TG |
| 362 | Cys-K19- | TGCGATTTGGGTAAGAAACTGCTAGAGGCTGCCAGAGCCGGACAAG |
| | 7D12- | ATGATGAGGTCAGAATACTTATGGCTAATGGCGCCGACGTGAATGC |
| | SOCS1pep | CTCAGACCGTTGGGGTTGGACACCATTGCACCTTGCCGCCTGGTGG |
| | | GGTCATTTGGAAATTGTAGAGGTCCTATTGAAGAGGGGAGCTGACG |
| | | TTTCCGCAGCCGACTTACACGGCCAGAGTCCTTTACATCTTGCTGC |
| | | TATGGTCGGCCACCTAGAGATCGTAGAGGTTCTTCTGAAGTACGGA |
| | | GCTGACGTGAATGCTAAAGATACTATGGGTGCCACCCCTCTGCACC |
| | | TTGCAGCAAGATCAGGACACCTGGAGATCGTAGAGGAGCTAGTAAA |
| | | GAACGGAGCTGATATGAATGCACAGGATAAGTTTGGCAAGACCACC |
| | | TTCGATATTTCAACCGATAACGGAAATGAGGATTTGGCTGAAATAC |
| | | TTCAAAAGCTAGGTGGCGGTGGTTCCGGCGGTGGCGGCTCTCAAGT |
| | | AAAGTTGGAAGAAAGTGGAGGTGGATCTGTTCAAACTGGCGGCTCC |
| | | CTTAGGCTTACTTGTGCCGCCTCCGGAAGGACGTCAAGGTCCTACG |
| | | GCATGGGCTGGTTTAGACAAGCTCCAGGCAAAGAGAGGGAGTTCGT |
| | | ATCCGGAATCTCATGGAGAGGTGATTCCACAGGATACGCTGACAGT |
| | | GTGAAGGGCAGATTCACAATTAGTCGTGATAATGCCAAGAACACAG |
| | | TGGATCTACAGATGAACTCCCTGAAGCCTGAAGATACCGCCATCTA |
| | | TTATTGTGCAGCCGCCGCTGGCTCTGCCTGGTACGGCACCTTATAT |
| | | GAGTACGACTACTGGGGACAGGGAACCCAAGTGACCGTGTCTTCTG |
| | | GCGGAGGTGGAAGTGTTAGACCTTTGCAGGAGCTGTGTAGACAGCG |
| | | TATTGTTGCAACAGTCGGACGTGAAAACTTAGCACGTATTCCACTG |
| | | AACCCCGTCCTTAGAGACTATCTTTCATCATTTCCCTTCCAAATA |
| 363 | Cys-K19- | GATCTGGGTAAAAAGCTACTAGAGGCTGCAAGAGCTGGCCAAGACG |
| | Mb-VHL₁₅₂₋₂₁₃ | ACGAAGTTCGTATCCTGATGGCTAATGGCGCAGATGTGAACGCATC |
| | | CGATCGTTGGGGCTGGACACCTCTACACCTTGCCGCATGGTGGGGC |
| | | CACTTGGAAATCGTTGAGGTTTTACTTAAACGTGGTGCAGACGTGT |
| | | CCGCCGCTGACCTACACGGTCAATCTCCACTGCATCTGGCTGCCAT |
| | | GGTAGGACACCTAGAAATCGTGGAGGTATTGTTGAAGTACGGCGCC |
| | | GATGTCAACGCTAAAGACACAATGGGTGCAACGCCATTACATCTTG |
| | | CCGCTAGATCAGGACACTTGGAGATCGTAGAGGAGCTGCTAAAGAA |
| | | TGGCGCTGACATGAATGCACAGGATAAGTTTGGTAAAACTACTTTT |
| | | GATATTTCCACAGACAACGGCAACGAGGATCTGGCCGAAATTTTGC |
| | | AGAAGCTAGGCGGCGGCGGATCTGAGCCCAAATCATCCGATAAAAC |
| | | ACACACCTGTCCTCCATGTCCTGGACAGCCAAGGGAGCCACAAGTC |
| | | TAGACCTTACCTCCTTCAAGAGAAGAAATGACTAAAAATCAAGTCT |
| | | CCTTGACTTGCTTAGTAAAGGGATTCTACCCATCTGACATCGCTGT |
| | | GGAGTGGGAGTCCAACGGCCAGCCAGAGAACAACTATAAGACTACG |
| | | CCTCCAGTGCTAGACAGTGATGGCTCCTTTTTTCTGTACTCCAAGT |
| | | TAACGGTTGATAAGTCTAGATGGCAACAGGGAAATGTATTCAGTTG |
| | | TTCCGTGATGCATGAAGCTCTGCACAATCACTATACCCAAAAGTCT |
| | | CTTTCATTAAGTCCAGGAGGCGGCGGAGGATCTACATTACCAGTTT |
| | | ACACGTTAAAAGAGAGGTGTTTGCAAGTTGTTCGTTCCCTTGTCAA |
| | | ACCCGAGAACTATAGACGTTTGGACATCGTCAGGTCTTTGTATGAG |
| | | GATCTGGAGGATCACCCCAACGTGCAAAAGGACCTAGAGAGATTGA |
| | | CACAAGAACGTATCGCACATCAAAGGATGGGCGAC |
| 364 | VHL152-213- | ACCTTGCCCGTCTATACCCTTAAGGAAAGGTGCCTGCAGGTAGTTA |
| | K19-Mb- | GATCCCTAGTAAAGCCCGAAAACTACAGAAGGTTGGACATCGTCAG |
| | Cys | ATCACTGTATGAAGACCTTGAGGACCATCCAAACGTGCAAAAAGAC |
| | | CTAGAAAGACTAACACAAGAGAGGATCGCTCACCAGAGAATGGGAG |
| | | ACGGAGGCGGAGGTAGTGATCTGGGAAAAAAGTTACTAGAAGCCGC |
| | | CAGAGCTGGACAAGATGATGAGGTAAGGATACTAATGGCAAATGGC |
| | | GCAGACGTGAACGCATCAGATAGGTGGGGCTGGACACCATTGCATC |
| | | TGGCCGCATGGTGGGGACACCTAGAAATCGTAGAAGTGTTGTTGAA |
| | | AAGGGGCGCTGATGTGTCTGCAGCCGACCTGCATGGACAATCACCC |
| | | CTGCACCTAGCTGCTATGGTTGGCCATTTAGAGATCGTTGAAGTGC |
| | | TACTGAAATATGGAGCAGATGTGAACGCCAAAGATACGATGGGTGC |
| | | TACACCTCTTCATTTAGCAGCAAGGTCAGGACACCTGGAAATAGTA |
| | | GAGGAGTTACTGAAAAATGGTGCTGACATGAACGCCCAGGACAAAT |
| | | TTGGAAAGACCACTTTCGATATATCTAGTGACAACGGTAATGAGGA |
| | | TCTTGCCGAGATACTGCAGAAGCTTGGAGGAGGAGGCTCCGAACCA |
| | | AAGTCTAGTGACAAGACGCACACGTGTCCTCCTTGTCCCGGCCAAC |
| | | CAAGAGAGCCTCAGGTTTACACGTTGCCTCCATCCAGGGAGGAAAT |
| | | GACCAAGAATCAGGTATCCTTAACATGTCTGGTGAAGGGTTTTTAT |
| | | CCTTCAGATATTGCCGTGGAGTGGGAATCTAACGGCCAACCAGAAA |
| | | ACAACTATAAAACTACTCCACCAGTGCTGGACTCCGACGGCTCATT |
| | | CTTCTTGTATTCAAAACTGACCGTAGACAAATCCCGTTGGCAGCAG |
| | | GGTAATGTTTTCTCCTGTTCAGTAATGCACGAAGCCCTGCACAACC |
| | | ACTATACACAAAAATCACTTTCCCTTTCTCCCGGTTGC |
| 365 | Cys-K19- | TGCGACTTGGGTAAGAAATTGCTAGAGGCAGCTAGAGCCGGTCAGG |
| | TRIM21₁₋₂₇₇ | ACGACGAGGTTCGTATCTTGATGGCTAACGGAGCTGACGTAAATGC |
| | | CTCCGACCGTTGGGGTTGGACGCCCCTACATCTGGCCGCATGGTGG |
| | | GGCCACCTGGAAATTGTAGAGGTTTTACTTAAGAGGGGCGCAGACG |
| | | TGAGTGCTGCTGACCTTCACGGACAGTCTCCCCTACATTTAGCAGC |
| | | AATGGTCGGTCACCTTGAGATCGTCGAGGTGCTTTTAAAGTATGGC |
| | | GCAGACGTCAATGCAAAGGACACTATGGGCGCCACTCCTCTTCATC |
| | | TTGCAGCCAGGTCTGGACACCTAGAAATAGTCGAGGAGCTACTAAA |
| | | AAATGGAGCTGAGATGAATGCCCAAGACAAGTTTGGTAAAACTACA |
| | | TTTGACATCTCTACAGACAATGGAAACGAGGATTTGGCTGAAATAC |
| | | TACAAAAACTTGGAGGAGGTGGCTCTGCCTCCGCCGCTAGGCTTAC |
| | | AATGATGTGGGAAGAGGTTACGTGTCCCATCTGCCTGGACCCCTTC |
| | | GTCGAGCCCGTCAGTATCGAATGCGGTCACTCTTTTTGTCAAGAAT |
| | | GTATAAGTCAAGTTGGTAAGGGAGGTGGCTCCGTGTGCCCAGTGTG |
| | | GAGGCAAAGGTTTCTGTTAAAAAATTTGCGTCCTAATAGACAAGTT |
| | | GCCAACATGGTAAACAATCTTAAAGAGATCTCCCAGGAAGCCAGGG |
| | | AGGGAACACAAGGAGAACGTTGTGCCGTTCATGGCGAACGTCTTCA |
| | | TTTGTTCTGTGAGAAAGATGGTAAGGCATTATGCTGGGTGTGTGCC |
| | | CAATCCCGTAAGCACCGTGACCATGCCATGGTACCCCTAGAAGAGG |
| | | CTGCCCAAGAATACCAAGAGAAATTGCAAGTGGCTCTTGGTGAGCT |
| | | TCGTAGAAAGCAGGAGCTAGCCGAGAAATTAGAAGTAGAGATCGCA |
| | | ATTAAGAGGGCTGATTGGAAGAAGACCGTCGAGACTCAGAAGTCTC |
| | | GTATTCACGCCGAGTTCGTCCAGCAAAAGAACTTTCTGGTAGAGGA |
| | | GGAGCAGCGTCAATTACAAGAGCTTGAGAAGGACGAACGTGAGCAG |
| | | CTTCGTATCCTGGGAGAAAAGGAAGCTAAACTTGCACAACAATCCC |
| | | AGGCCCTTCAAGAGCTGATTAGTGAGTTGGATCGTAGGTGTCACTC |
| | | CTCTGCCTTAGAACTATTGCAAGAAGTAATCATCGTCCTAGAGCGT |
| | | TCAGAATCTTGGAACCTGAAGGACTTGGATATAACATCACCAGAGC |
| | | TTCGTTCAGTCTGTCATGTACCTGGA |
| 366 | TRIM21₁₋₂₇₇ | ATGGCTAGTGCAGCCCGTTTAACAATGATGTGGGAGGAAGTCACAT |
| | -K19- | GCCCAATATGTCTTGACCCTTTCGTCGAGCCTGTATCTATCGAATG |
| | Cys | TGGCCATTCTTTCTGCCAAGAGTGTATCTCTCAGGTTGGCAAGGGC |
| | | GGAGGATCCGTTTGCCCCGTTTGCAGGCAGCGTTTTTTGCTAAAAA |
| | | ACTTGAGGCCAAACCGTCAACTGGCTAACATGGTGAATAATTTAAA |
| | | GGAAATTAGTCAGGAGGCAAGAGAGGGAACACAAGGAGAGAGATGC |
| | | GCCGTGCACGGCGAAAGACTGCATCTTTTTTGCGAAAAAGATGGTA |
| | | AGGCCCTTTGTTGGGTCTGTGCACAATCCAGGAAGCACCGTGATCA |
| | | CGCAATGGTCCCCCTAGAAGAGGCAGCTCAAGAGTATCAGGAGAAA |
| | | TTACAGGTCGCTTTGGGCGAACTGCGTCGTAAACAGGAACTTGCAG |
| | | AGAAACTTGAAGTAGAAATCGCTATTAAGCGTGCCGATTGGAAGAA |
| | | AACTGTCGAGACACAGAAGTCTAGAATTCATGCTGAGTTCGTTCAA |
| | | CAAAAGAACTTTTTGGTTGAAGAGGAACAGCGTCAGCTACAAGAAT |
| | | TAGAGAAGGACGAGAGAGAGCAGCTTCGTATCCTTGGTGAGAAGGA |
| | | GGCCAAGCTGGCTCAACAGTCCCAAGCCCTACAAGAACTAATTTCC |
| | | GAACTGGACAGAAGATGCCACTCATCTGCATTAGAGCTAGTTCAGG |
| | | AAGTCATTATTGTAGTGGAAAGAAGTGAATCCTGGAACCTGAAGGA |
| | | TTTGGATATTACCAGTCCTGAACTAAGAAGTGTGTGCCACGTACCA |
| | | GGAGGAGGCGGAGGCTCCGACTTAGGAAAGAAACTACTGGAGGCCG |
| | | CTAGGGCTGGACAAGATGACGAAGTAAGGATTTTGATGGCAAACGG |
| | | AGCCGACGTTAATGCCAGTGATAGATGGGGATGGACACCATTGCAC |
| | | CTGGCTGCATGGTGGGGACATCTGGAGATTGTCGAGGTACTGTTGA |
| | | AACGTGGAGCCGATGTATCAGCAGCTGATCTACATGGACAGTCTCC |
| | | CTTGCACTTGGCCGCAATGGTTGGACATCTTGAAATTGTGGAAGTT |
| | | TTGTTAAAATATGGCGCTGACGTTAATGCAAAGGACACCATGGGAG |
| | | CCACGCCCTTAGATCTAGCAGCCAGATCCGGTCATTTAGAGATAGT |
| | | CGAAGAATTACTGAAAAATGGTGCCGAGATGAATGCCCAAGACAAA |
| | | TTCGGAAAAACGACCTTTGATATATCTACCGATAACGGTAACGAAG |
| | | ATTTAGCAGAAATACTTCAGAAGCTGTGC |
| 367 | Cys-7D12- | TGTCAAGTCAAACTAGAAGAAAGTGGAGGTGGCTCCGTCCAGACTG |
| | K19- | GTGGATCATTGAGGCTTACATGCGCTGCTTCTGGCAGAACATCAAG |
| | TRIM21₁₋₂₇₇ | GTCATATGGTATGGGTTGGTTTCGTCAGGCACCTGGCAAAGAGAGG |
| | | GAGTTCGTCTCTGGAATTAGTTGGCGTGGAGATTCTACCGGCTACG |
| | | CTGATTCAGTCAAGGGACGTTTCACTATATCTAGAGACAATGCAAA |
| | | AAATACCGTGGATCTTCAGATGAACTCACTGAAGCCTGAAGACACC |
| | | GCTATCTACTATTGTGCTGCCGCCGCTGGTTCAGCCTGGTACGGCA |
| | | CCCTATATGAATACGACTATTGGGGCCAGGGCACCCAGGTCACTGT |
| | | ATCCTCTGGAGGTGGCGGTTCTGGAGGCGGTGGCTCCGATCTGGGT |
| | | AAGAAGCTGCTTGAAGCAGCACGTGCCGGTCAAGACGATGAAGTAA |
| | | GGATTTTGATGGCTAACGGCGCTGATGTTAATGCATCAGATAGGTG |
| | | GGGCTGGACCCCTTTGCACCTAGCAGCTTGGTGGGGCCACCTTGAG |
| | | ATAGTCGAAGTTCTATTGAAGCGTGGCGCCGACGTGTCCGCCGCAG |
| | | ATTTACACGGCCAATCACCCCTGCACTTGGCTGCCATGGTGGGACA |
| | | CTTAGAGATCGTTGAGGTGCTACTGAAGTATGGAGCCGATGTTAAC |
| | | GCAAAGGATACTATGGGCGCTACTCCCCTACACTTAGCTGCACGTA |
| | | GTGGCCATTTAGAGATAGTGGAAGAACTATTAAAGAACGGAGCAGA |
| | | CATGAACGCACAAGATAAGTTCGGTAAGACCACATTCGATATTTCT |
| | | ACGGACAATGGCAATGAAGACCTGGCCGAAATACTACAAAAGCTGG |
| | | GAGGCGGTGGATCTGCATCCGCCGCTAGGTTGACTATGATGTGGGA |
| | | AGAAGTTACATGTCCAATCTGTCTAGATCCTTTCGTTGAACCTGTC |
| | | TCTATTGAATGCGGCCACTCATTTTGTCAAGAGTGTATTAGTCAGG |
| | | TGGGTAAGGGCGGTGGCTCAGTGTGTCCAGTCTGTAGGCAGAGATT |
| | | TTTGCTGAAGAATTTGAGGCCTAACCGTCAGTTGGCCAATATGGTT |
| | | AACAACCTGAAAGAGATATCCCAAGAGGCCAGAGAGGGCACTCAGG |
| | | GCGAGAGATGTGCCGTACACGGTGAGAGGCTGCACCTTTTCTGCGA |
| | | AAAAGACGGCAAGGCTTTATGCTGGGTCTGCGCCCAGTCCAGGAAA |
| | | CACAGAGACCATGCCATGGTGCCATTAGAAGAAGCAGCTCAGGAGT |
| | | ATCAGGAGAAGCTGCAAGTAGCACTTGGAGAGCTTAGGCGTAAGCA |
| | | GGAGCTAGCAGAGAAACTTGAGGTTGAGATAGCCATCAAGAGGGCT |
| | | GATTGGAAAAAAACAGTTGAGACTCAGAAGTCCCGTATACACGCTG |
| | | AGTTCGTACAACAAAAGAACTTTTTGGTCGAAGAGGAGCAGCGTCA |
| | | ACTGCAGGAACTTGAGAAGGACGAGAGGGAGCAGTTACGTATTCTA |
| | | GGCGAAAAGGAAGCAAAACTGGCCCAACAATCCCAAGCTCTTCAAG |
| | | AGCTTATTAGTGAGCTAGACAGACGTTGTCACTCTTCAGCCCTTGA |
| | | GCTGCTACAGGAAGTTATCATCGTACTTGAAAGGAGTGAATCCTGG |
| | | AATTTGAAGGACTTGGATATCACATCTCCAGAATTGAGGTCTGTCT |
| | | GTCATGTGCCTGGA |
| 368 | BC2-Fc_{S239C} | CAAGTACAATTGGTTGAGTCTGGCGGAGGATTAGTCCAGCCTGGTG |
| | | GATCTCTTACACTTTCTTGTACCGCTTCCGGCTTCACACTAGATCA |
| | | CTACGACATAGGTTGGTTCAGACAGGCTCCCGGCAAAGAGAGAGAA |
| | | GGTGTCTCCTGTATAAATAACTCTGACGATGACACGTATTACGCTG |
| | | ATAGTGTCAAGGGTAGGTTCACCATTTTCATGAATAACGCAAAGGA |
| | | GAGGGTTTACCTACAAATGAATTCTCTGAAACCTGAAGACACAGCC |
| | | ATTTACTACTGTGCTGAAGCCAGAGGATGTAAACGTGGTCGTTATG |
| | | AGTACGACTTCTGGGGCCAGGGAACACAAGTTACTGTCTCCTCCGG |
| | | CGGAGGAGGTAGTGATAAGACCCATACTTGTCCCCCTTGCCCTGCA |
| | | CCTGAGCTGTTGGGCGGACCATGCGTGTTCTTGTTTCCACCCAAGC |
| | | CAAAAGATACCTTAATGATTTCACGTACCCCCGAGGTCACCTGCGT |
| | | TGTGGTTGATGTGTCACACGAAGATCCAGAGGTGAAGTTTAACTGG |
| | | TACGTTGATGGTGTTGAAGTCCATAACGCTAAAACCAAACCAAGGG |
| | | AGGAACAGTACGCCTCTACATACCGTGTCGTGTCAGTTCTGACCGT |
| | | GTTACACCAAGATTGGCTTAACGGCAAAGAATACAAGTGTAAAGTC |
| | | TCAAACAAGGCTTTACCCGCTCCAATAGAGAAAACCATATCAAAAG |
| | | CTAAGGGCCAGCCTCGTGAACCTCAGGTTTACACTCTTCCACCATC |
| | | CAGGGAAGAAATGACCAAAAATCAAGTTTCTCTAACCTGCTTAGTT |
| | | AAGGGTTTTTACCCTTCAGACATAGCAGTCGAGTGGGAGTCTAATG |
| | | GCCAGCCCGAAAACAACTATAAAACTACTCCTCCCGTGCTTGATAG |
| | | TGACGGTTCTTTCTTCTTATATTCCAAATTGACTGTCGATAAATCC |
| | | AGGTGGCAACAAGGTAACGTCTTTTCATGCTCCGTAATGCATGAGG |
| | | CTCTTCATAATCACTAGACGCAAAAATCCCTATCCCTTAGTCCAGG |
| | | A |
| 369 | Cys-BC2- | TGTCAGGTACAACTTGTAGAATCCGGCGGCGGTCTTGTCCAGCCAG |
| | Mb-VHL₁₅₂₋₂₁₃ | GCGGCTCCCTGACGCTTTCCTGTACCGCAAGTGGCTTTACTCTAGA |
| | | CCATTATGACATTGGCTGGTTCAGGCAGGCCCCTGGCAAGGAGAGA |
| | | GAGGGCGTATCCTGTATTAATAACTCCGACGACGACACCTATTATG |
| | | CAGATAGTGTAAAAGGCAGGTTTACCATCTTCATGAACAACGCAAA |
| | | AGACACAGTATACCTACAAATGAATAGTCTGAAGCCAGAGGATACA |
| | | GCCATTTATTATTGTGCAGAAGCAAGAGGCTGCAAAAGGGGACGTT |
| | | ATGAGTACGATTTCTGGGGACAAGGCACCCAAGTTACGGTATCATC |
| | | AGAGCCAAAATCCTCAGATAAAACACACACATGCCCCCCTTGTCCC |
| | | GGCCAACCAAGAGAGCCCCAGGTGTATACGCTGCCCCCAAGTAGGG |
| | | AAGAAATGACTAAAAACCAAGTTTCTTTAACGTGCTTAGTTAAGGG |
| | | ATTTTACCCTTCAGACATAGCCGTTGAGTGGGAGTCTAACGGCCAA |
| | | CCTGAGAACAACTATAAAACTACCCCTCCCGTCTTAGATTCAGATG |
| | | GTTCATTTTTTCTGTACTCAAAGCTAACGGTCGATAAGTCAAGGTG |
| | | GCAGCAAGGCAACGTATTTAGTTGCTCTGTAATGCATGAGGCCCTT |
| | | CACAACCATTATACTCAGAAAAGTCTATCTCTTTCTCCTGGTGGTG |
| | | GCGGCGGCTCAGGCGGCGGTGGTTCCACTCTTCCCGTCTATACCTT |
| | | AAAGGAGCGTTGCCTACAGGTAGTGAGAAGTTTAGTTAAGCCTGAG |
| | | AATTACAGGAGATTAGACATTGTGCGTTCATTATACGAGGACCTAG |
| | | AGGATCATCCCAACGTGCAAAAGGATCTGGAGAGACTTACGCAGGA |
| | | GAGGATAGCTCATCAACGTATGGGTGAC |
| 370 | VHL₁₅₂₋₂₁₃- | ACACTACCAGTTTATACGCTGAAAGAGAGATGCCTGCAAGTAGTCC |
| | BC2-Mb- | GTTCTTTGGTCAAACCAGAGAACTATAGACGTCTAGACATCGTAAG |
| | Cys | GTCTCTTTATGAGGATCTTGAAGATCATCCCAACGTTCAGAAGGAC |
| | | TTGGAGAGATTAACCCAGGAACGTATCGCACACCAGAGAATGGGCG |
| | | ACGGTGGCGGTGGCTCCGGTGGTGGTGGCTCACAGGTTCAGCTAGT |
| | | AGAATCAGGTGGAGGTCTGGTTCAACCCGGCGGTTCCCTAACTTTG |
| | | TCCTGCACCGCATCTGGATTCACCCTTGACCACTATGACATAGGCT |
| | | GGTTTCGTCAGGCCCCCGGCAAAGAGAGAGAGGGAGTTAGTTGTAT |
| | | AAACAACTCAGATGATGACACTTATTACGCAGATTCAGTGAAGGGC |
| | | CGTTTTACTATCTTCATGAATAATGCTAAAGATACCGTATACCTAC |
| | | AAATGAACTCTTTGAAACCCGAGGACACGGCCATATACTATTGCGC |
| | | CGAAGCCCGTGGTTGCAAGCGTGGCAGATACGAATATGACTTTTGG |
| | | GGTCAGGGAACCCAGGTCACAGTTAGTTCAGAGCCTAAATCTAGTG |
| | | ATAAGACCCATACGTGCCCACCCTGCCCAGGCCAACCCAGGGAACC |
| | | TCAGGTGTATACTTTGCCCCCAAGTAGAGAGGAAATGACAAAGAAT |
| | | CAGGTGTCACTAACCTGTCTTGTTAAGGGCTTCTATCCATCTGACA |
| | | TAGCCGTCGAATGGGAGAGTAATGGACAACCCGAGAATAACTATAA |
| | | AACTACGCCTCCCGTATTGGACTCTGATGGATCATTCTTTTTATAC |
| | | AGTAAATTAACGGTAGATAAGTCCAGGTGGCAGCAGGGAAACGTCT |
| | | TCTCTTGTTCTGTGATGCACGAAGCACTGCACAACCATTACACTCA |
| | | AAAATCTTTGAGTTTGTCACCAGGCTGC |
| 371 | Cys- | TGCACTCTACCAGTCTACACACTTAAAGAAAGATGTTTACAGGTTG |
| | VHL₁₅₂₋₂₁₃- | TGCGTTCACTGGTGAAGCCAGAAAATTACCGTCGTTTAGACATCGT |
| | BC2- | GAGGTCCTTATACGAAGATCTGGAAGATCATCCTAACGTACAAAAA |
| | SOCS1pep | GATCTTGAGCGTCTTACTCAGGAGCGTATAGCACATCAGAGGATGG |
| | | GAGACGGAGGAGGAGGCAGTGGTGGAGGTGGCTCACAAGTGCAGCT |
| | | AGTGGAATCCGGTGGCGGATTAGTTCAGCCAGGCGGCTCACTGACT |
| | | CTATCTTGTACGGCAAGTGGTTTCACGTTAGATCATTACGACATCG |
| | | GTTGGTTCAGGCAAGCTCCTGGCAAGGAGCGTGAAGGCGTCTCCTG |
| | | TATCAATAACAGTGACGATGACACGTACTATGCCGATTCAGTGAAG |
| | | GGTCGTTTTACGATCTTCATGAACAATGCCAAGGATACGGTCTACT |
| | | TGCAAATGAACAGTTTGAAGCCCGAGGATACCGCTATATATTATTG |
| | | CGCTGAGGCCCGTGGCTGCAAAAGGGGTAGGTATGAATACGATTTC |
| | | TGGGGACAGGGAACTCAGGTCACGGTTAGTAGTGGAGGAGGTGGCA |
| | | GTGGCGGAGGCGGTTCAGTGAGACCCCTTCAGGAGTTGTGCCGTCA |
| | | GCGTATCGTCGCAACAGTTGGAAGAGAAAATCTAGCTCGTATACCA |
| | | TTGAATCCAGTTCTAAGGGATTACTTGTCTTCCTTCCCCTTTCAGA |
| | | TT |
| 372 | VHL₁₅₂₋₂₁₃ | ACGTTGCCAGTATATACTCTTAAAGAGAGGTGCTTACAGGTCGTTC |
| | _{(Y185F)}-BC2- | GTTCTCTAGTTAAACCCGAGAATTATAGGAGACTGGACATCGTGCG |
| | Cys | TAGTCTGTTCGAGGATCTGGAGGACCATCCCAACGTCCAAAAGGAT |
| | | CTAGAAAGACTAACGCAAGAAAGAATTGCTCATCAAAGGATGGGAG |
| | | ATGGCGGTGGTGGCTCAGGTGGAGGCGGATCACAAGTTCAGCTTGT |
| | | CGAGTCAGGCGGTGGACTGGTACAGCCTGGAGGATCCCTGACGTTA |
| | | TCATGCACAGCCTCCGGTTTTACCTTAGACCACTACGATATTGGCT |
| | | GGTTTAGGCAGGCACCCGGTAAAGAGAGGGAAGGTGTGTCCTGTAT |
| | | AAATAATTCTGACGATGATACATACTACGCCGATTCAGTGAAAGGT |
| | | CGTTTCACAATTTTTATGAACAACGCCAAAGATACGGTGTACCTAC |
| | | AAATGAACAGTCTAAAACCAGAAGACACGGCCATCTACTACTGTGC |
| | | CGAAGCTCGTGGCTGTAAAAGAGGACGTTATGAGTATGATTTCTGG |
| | | GGTCAAGGTACTCAAGTTACGGTATCATCCTGT |
| 373 | Cys-BC2- | TGTCAGGTTCAACTGGTGGAGTCTGGCGGCGGCTTGGTTCAACCCG |
| | TRIM21₁₋₂₇₇ | GAGGCTCTTTGACCTTGTCTTGCACCGCTTCCGGTTTTACCCTTGA |
| | | CCATTATGATATAGGATGGTTCAGGCAAGCCCCCGGTAAAGAGCGT |
| | | GAGGGAGTATCCTGCATCAACAATTCCGACGACGACACGTAGTATG |
| | | CCGACTCCGTGAAGGGTAGGTTTACCATTTTTATGAACAATGCTAA |
| | | GGATACGGTATATCTGCAAATGAATTCCTTGAAGCCCGAGGACACC |
| | | GCCATCTATTAGTGTGCAGAAGCTAGAGGCTGTAAAAGAGGAAGAT |
| | | ACGAGTACGACTTCTGGGGTCAGGGCACTCAGGTAACCGTCTCCTC |
| | | TGGAGGAGGAGGCAGTGGAGGAGGTGGATCTGCATCAGCAGCCCGT |
| | | CTGACTATGATGTGGGAGGAAGTCACATGTCCCATATGTTTAGACC |
| | | CCTTCGTAGAGCCTGTCTCAATCGAATGCGGACATTCCTTTTGTCA |
| | | AGAGTGTATCTCTCAGGTTGGTAAGGGAGGTGGCTCTGTCTGCCCC |
| | | GTCTGCCGTCAAAGGTTCCTATTGAAGAACCTGAGGCCAAATCGTC |
| | | AGTTGGCTAACATGGTGAATAACCTTAAAGAGATCTCCCAAGAAGC |
| | | CAGAGAAGGCACGCAAGGCGAACGTTGCGCAGTTCATGGCGAGAGA |
| | | CTACATCTATTCTGCGAGAAGGACGGTAAAGCATTGTGCTGGGTCT |
| | | GTGCACAGAGTAGGAAGCATAGGGATCACGCAATGGTGCCACTTGA |
| | | AGAAGCCGCCCAGGAATATCAGGAAAAACTGCAGGTAGCTTTAGGC |
| | | GAATTGAGACGTAAGCAAGAATTGGCAGAGAAGCTGGAGGTAGAGA |
| | | TAGCAATAAAGCGTGCCGATTGGAAAAAAACTGTGGAAACGCAGAA |
| | | AAGTAGAATACACGCAGAATTCGTTCAACAAAAAAATTTCCTAGTA |
| | | GAGGAGGAGCAGAGGCAACTGCAGGAGTTGGAAAAGGACGAACGTG |
| | | AGCAACTAAGAATACTTGGCGAAAAGGAAGCTAAGTTGGCCCAGCA |
| | | AAGTCAAGCTTTACAAGAGTTGATTTCCGAATTAGATCGTCGTTGT |
| | | CACTCTTCAGCTCTTGAGCTATTGCAAGAAGTTATCATCGTGCTGG |
| | | AGAGATCAGAGTCATGGAATCTAAAAGATTTGGACA |
| 374 | TRIM21₁₋₂₇₇ | CATCATCACCACCATCATGGAGATGATGATGATAAGGCATCAGCAG |
| | -BC2-Cys | CACGTCTAACAATGATGTGGGAGGAAGTCACTTGTCCAATATGTCT |
| | | TGATCCCTTCGTAGAGCCTGTTTCAATAGAGTGTGGACATTCCTTT |
| | | TGTCAAGAATGCATTAGTCAGGTAGGTAAAGGAGGTGGTTCTGTTT |
| | | GTCCTGTATGCAGACAAAGATTCTTATTAAAGAACCTACGTCCCAA |
| | | CAGACAGTTAGCAAATATGGTCAACAATCTGAAAGAGATTTCTCAA |
| | | GAGGCCCGTGAAGGAACACAGGGTGAGAGATGCGCTGTCCACGGAG |
| | | AGCGTCTTCATTTATTTTGCGAGAAGGATGGTAAAGCACTTTGCTG |
| | | GGTATGTGCTCAATCACGTAAACATCGTGACCACGCCATGGTTCCC |
| | | CTAGAAGAAGCTGCCCAAGAGTATCAGGAGAAACTACAAGTGGCAC |
| | | TTGGCGAACTGAGGAGGAAGCAAGAGTTGGCCGAAAAACTTGAGGT |
| | | CGAAATTGCCATTAAGAGGGCCGATTGGAAGAAGACTGTCGAGACA |
| | | CAGAAAAGTAGAATTCATGCAGAATTTGTGCAGCAAAAAAACTTCT |
| | | TGGTTGAAGAGGAGCAGCGTCAATTACAGGAATTGGAAAAAGATGA |
| | | AAGGGAACAGCTTCGTATATTAGGAGAAAAGGAGGCTAAACTTGCC |
| | | CAACAGTCACAGGCTCTGCAGGAGCTAATCTCCGAACTTGACAGAC |
| | | GTTGCCACTCCAGTGCTTTAGAGCTTTTGCAGGAGGTCATCATAGT |
| | | CCTGGAGAGAAGTGAATCATGGAACCTTAAGGATCTGGACATTACC |
| | | TCACCCGAACTAAGATCCGTCTGCCATGTTCCAGGTGGCGGTGGCG |
| | | GATCAGGTGGTGGCGGATCACAGGTACAGCTAGTGGAGAGTGGTGG |
| | | AGGCTTAGTGCAACCAGGTGGATCACTAACTTTATCATGCACTGCC |
| | | AGTGGCTTCACACTGGACCACTACGACATTGGTTGGTTTAGACAGG |
| | | CCCCCGGAAAAGAAAGGGAAGGCGTATCCTGTATCAACAATTCCGA |
| | | CGACGACACCTATTATGCTGATTCCGTAAAAGGTAGGTTTACGATT |
| | | TTTATGAACAATGCCAAGGATACGGTGTATCTACAGATGAATTCCC |
| | | TTAAACCTGAAGATACCGCAATTTATTACTGTGCAGAGGCCAGGGG |
| | | TTGCAAAAGAGGCAGGTACGAATATGACTTCTGGGGACAAGGCACC |
| | | CAAGTAACAGTCTCCTCCTGC |
| 375 | VHL₁₅₂₋₂₁₃- | ACCCTACCCGTGTACACCTTAAAGGAGAGGTGTCTACAGGTGGTTA |
| | BC2- | GAAGTCTAGTGAAACCAGAGAACTATAGAAGGCTTGATATCGTGCG |
| | TRIM21₁₋₂₇₇ | TTCCTTGTACGAAGACTTGGAGGACCACCCAAATGTTCAGAAGGAC |
| | -Cys | TTGGAAAGGTTGACCCAAGAGCGTATCGCACACCAACGTATGGGCG |
| | | ATGGAGGCGGTGGCAGTGGAGGAGGAGGCTCTCAAGTACAGTTAGT |
| | | GGAATCAGGTGGCGGTTTGGTCCAACCAGGCGGAAGTCTTACTTTG |
| | | TCCTGCACGGCATCCGGTTTCACTTTGGACCACTATGATATCGGCT |
| | | GGTTCCGTCAAGCCCCTGGAAAAGAAAGAGAGGGCGTATCTTGTAT |
| | | CAATAACAGTGACGACGATACATATTACGCCGACTCTGTAAAGGGA |
| | | AGATTCACCATTTTCATGAACAACGCCAAAGACACCGTATACCTTC |
| | | AGATGAATTCCTTGAAGCCAGAGGATACAGCTATCTATTATTGCGC |
| | | TGAGGCTCGTGGCTGCAAAAGGGGAAGATATGAGTACGACTTCTGG |
| | | GGCCAAGGCACGCAAGTTACAGTCTCCAGTGGTGGCGGAGGCTCTG |
| | | GTGGAGGAGGATCTGCTTCTGCAGCAAGGTTGACCATGATGTGGGA |
| | | GGAGGTGACGTGTCCCATTTGTCTTGATCCATTCGTCGAGCCCGTG |
| | | TCCATAGAATGTGGACACTCTTTCTGCCAGGAGTGTATTTCCCAGG |
| | | TCGGCAAAGGTGGTGGATCAGTGTGTCCAGTCTGCCGTCAGAGATT |
| | | CCTATTGAAGAATCTGAGACCCAACAGACAGCTAGCTAACATGGTT |
| | | AATAACTTAAAAGAAATCAGTCAAGAAGCAAGAGAGGGTACGCAGG |
| | | GAGAGCGTTGCGCAGTTCACGGAGAGAGACTACACCTATTCTGCGA |
| | | AAAGGACGGCAAGGCATTGTGTTGGGTGTGTGCACAAAGTCGTAAG |
| | | CACAGAGATCATGCTATGGTGCCTTTGGAAGAGGCCGCCCAAGAGT |
| | | ATCAGGAGAAGTTGCAAGTCGCCTTGGGAGAGTTAAGAAGGAAGCA |
| | | GGAGTTAGCTGAAAAACTAGAGGTCGAGATCGCCATAAAGAGAGCA |
| | | GACTGGAAAAAGACGGTCGAGACACAAAAGTCAAGGATACACGCCG |
| | | AATTCGTGCAGCAGAAGAACTTCCTAGTAGAAGAAGAGCAAAGGCA |
| | | ATTGCAAGAGCTAGAAAAGGATGAAAGAGAACAGCTAAGGATTCTA |
| | | GGCGAGAAGGAAGCCAAGCTGGCTCAACAATCTCAAGCCCTACAAG |
| | | AGTTGATTAGTGAGCTAGACCGTAGATGCCATTCTTCCGCCCTTGA |
| | | ACTACTACAAGAAGTCATCATTGTTTTGGAAAGGTCTGAGTCTTGG |
| | | AACCTGAAGGATCTTGACATAACCAGTCCCGAACTACGTTCTGTTT |
| | | GTCACGTACCCGGTTGT |
| 376 | VHL₁₅₂₋₂₁₃- | ACCCTGCCTGTTTACACGCTAAAGGAGAGATGCCTGCAAGTTGTCA |
| | VHH^{CARD}_ | GATCATTAGTAAAGCCAGAGAATTATAGAAGGCTGGATATTGTAAG |
| | Mb-Cys | GTGAGTATATGAGGACTTAGAGGATCATCCCAATGTCCAAAAGGAT |
| | | CTTGAAAGGCTAACACAGGAGCGTATAGCACACCAGAGGATGGGAG |
| | | ATGGCGGAGGTGGCAGTCAGCTTCAGCTAGTAGAAAGTGGAGGTGG |
| | | TCTTGTGCAGCCTGGTGGATCCTTAAAGCTGTCCTGCGCCGCATCA |
| | | GGCTTCACATTCTCTAGGTATGCCATGAGTTGGTACAGGCAGGCCC |
| | | CCGGTAAAGAGCGTGAAAGTGTAGCTAGGATCTCCTCCGGTGGAGG |
| | | AACGATATACTATGCCGATTCTGTAAAGGGCAGGTTTACAATCTCC |
| | | CGTGAAGACGCAAAGAACACAGTCTATTTGCAGATGAATTCACTTA |
| | | AGCCTGAAGATACGGCTGTATATTATTGCTACGTGGGCGGCTTTTG |
| | | GGGTCAGGGAACTCAGGTGACCGTCTCCTCTGAGCCTAAAAGTAGT |
| | | GATAAGACGCATACCTGTCCTCCTTGTCCTGGACAGCCAAGAGAGC |
| | | CACAGGTCTATACACTGCCACCTTCAAGGGAAGAGATGACGAAGAA |
| | | CCAGGTAAGTCTGACCTGCCTTGTGAAGGGCTTTTACCCTTCTGAT |
| | | ATTGCTGTGGAATGGGAATCCAACGGTCAACCCGAAAACAACTATA |
| | | AGACAACACCACCCGTACTTGATTCCGATGGTAGTTTTTTCTTGTA |
| | | TAGTAAGCTGACTGTGGATAAATCCAGATGGCAACAGGGAAACGTA |
| | | TTCTCCTGCTCCGTAATGCATGAGGCTCTGCACAACCATTATACTC |
| | | AGAAATCTTTAAGTCTATCACCAGGCTGT |
| 377 | Cys- | TGTCAGTTGCAGTTGGTAGAATCCGGTGGCGGTCTAGTTCAACCAG |
| | ASC^{VHH}_ | GCGGATCATTGAAACTTTCATGTGCTGCATCCGGATTTACATTCTC |
| | VHLpep | TCGTTATGCTATGTCTTGGTATAGACAGGCACCTGGAAAGGAGAGG |
| | | GAATCCGTTGCTCGTATTAGTTCCGGTGGAGGTACGATCTATTACG |
| | | CCGACTCAGTAAAAGGTAGGTTTACCATAAGTAGAGAAGATGCAAA |
| | | GAACACAGTTTACTTGCAAATGAACAGTTTAAAGCCCGAAGACACT |
| | | GCAGTGTACTACTGCTATGTCGGCGGCTTTTGGGGCCAAGGAACGC |
| | | AGGTCACCGTTAGTTCTGGAGGAGGTGGTTCTCTGCCAGTCTATAC |
| | | TCTAAAGGAGAGATGCTTGCAGGTAGTCAGGTCATTAGTTAAACCA |
| | | GAAAACTACAGGAGACTTGATATAGTACGTTCTTTATACGAGGATT |
| | | TAGAAGACCATCCTAACGTG |
| 378 | VHL₁₅₂₋₂₁₃- | ACGCTACCCGTCTACACGTTGAAAGAGAGGTGTCTTCAGGTCGTTC |
| | ASC^{VHH}_ | GTTCTTTAGTAAAGCCCGAAAACTACAGGAGGCTGGACATCGTAAG |
| | Cys | ATCCCTGTATGAGGATCTGGAAGACCACCCCAATGTACAAAAAGAC |
| | | TTAGAGAGATTAACGCAGGAGAGAATCGCCCACCAGCGTATGGGTG |
| | | ACGGTGGTGGCGGAAGTCAACTTCAGCTAGTTGAATCTGGTGGCGG |
| | | ACTGGTCCAGCCTGGCGGTTCTCTAAAGCTTAGTTGTGCCGCTAGT |
| | | GGATTTACATTTTCAAGGTATGCTATGTCCTGGTACAGACAGGCTC |
| | | CCGGCAAGGAGAGAGAATCCGTTGCTAGAATATCTTCCGGTGGTGG |
| | | CACTATCTACTACGCTGATTCTGTCAAAGGTCGTTTCACTATATCT |
| | | AGGGAAGATGCCAAGAATACCGTCTATTTGCAAATGAATAGTCTGA |
| | | AGCCCGAAGACACTGCCGTCTACTACTGTTATGTTGGAGGCTTCTG |
| | | GGGTCAAGGTACGCAGGTTACAGTTAGTTCATGT |
| 379 | Cys- | TGTACCCTGCCTGTGTACACTCTAAAAGAAAGGTGTCTTCAGGTCG |
| | VHL₁₅₂₋₂₁₃- | TTAGATCCCTTGTAAAGCCAGAAAATTATAGGAGATTAGATATCGT |
| | ASC^{VHH}_ | GCGTTCACTTTACGAGGAGCTGGAGGAGCATCCAAACGTTCAGAAG |
| | SOCS1pep | GACCTAGAGAGACTGACCCAAGAGAGGATTGCTCACCAGAGAATGG |
| | | GCGACGGTGGAGGAGGATCTCAACTGCAATTAGTGGAGTCAGGTGG |
| | | CGGCTTAGTGCAACCAGGAGGCAGTTTGAAGCTAAGTTGTGCCGCA |
| | | TCCGGCTTTACCTTTTCTCGTTACGCAATGTCATGGTATCGTCAGG |
| | | CTCCAGGAAAGGAACGTGAGAGTGTAGCCCGTATCTCATCTGGAGG |
| | | TGGCACCATCTACTATGCCGATTCAGTGAAGGGTAGGTTCACAATT |
| | | TCTCGTGAAGACGCAAAGAACACGGTGTACCTACAAATGAATAGTT |
| | | TGAAGCCAGAAGACACGGCCGTCTATTATTGCTATGTCGGTGGTTT |
| | | TTGGGGTCAAGGAACGCAAGTTACAGTATCATCCGGCGGCGGCGGC |
| | | TCTGTAAGGCCATTGCAGGAACTATGTAGGCAACGTATTGTTGCCA |
| | | CGGTGGGTAGAGAGAACCTGGCCAGGATACCCTTAAATCCAGTGCT |
| | | TCGTGACTATCTGTCTTCCTTTCCATTTCAGATC |
| 380 | SOCS1pep- | GTTCGTCCTCTTCAAGAACTATGCCGTCAGAGAATAGTAGCTACTG |
| | VHL₁₅₂₋₂₁₃- | TGGGTAGGGAGAACCTTGCTAGAATTCCTCTGAATCCAGTTTTAAG |
| | ASC^{VHH}_ | AGACTATTTGTCCTCCTTTCCATTCCAGATCGGAGGCGGCGGAAGT |
| | Cys | ACTCTTCCCGTGTACACGCTGAAGGAAAGGTGTTTACAGGTCGTGA |
| | | GGTCATTGGTGAAGCCCGAGAATTATAGAAGACTAGACATAGTACG |
| | | TTCCCTGTATGAGGATTTAGAGGATCATCCAAATGTTCAGAAGGAT |
| | | TTAGAAAGACTGACGCAGGAAAGAATAGCCCACCAAAGAATGGGTG |
| | | ACGGTGGTGGAGGCTCACAACTACAACTTGTAGAGTCCGGTGGTGG |
| | | ATTAGTCCAGCCCGGAGGATCTCTAAAACTATCCTGCGCTGCATCT |
| | | GGTTTCACTTTTTCAAGGTACGCCATGTCTTGGTATAGACAGGCAC |
| | | CAGGTAAAGAGCGTGAGTCCGTCGCCAGAATTTCCTCAGGCGGTGG |
| | | TACGATCTACTATGCTGATTCTGTGAAAGGTAGGTTTACAATATCA |
| | | AGGGAGGATGCAAAGAACACTGTCTACCTACAGATGAATTCACTGA |
| | | AACCTGAAGACACAGCCGTTTACTATTGTTATGTTGGAGGTTTTTG |
| | | GGGACAGGGAACTCAAGTGACCGTATCTTCATGT |
| 381 | Cys- | CAATTGCAATTGGTGGAATCTGGCGGTGGCTTAGTACAGCCTGGTG |
| | ASC^{VHH}_ | GATCTTTGAAGCTATCTTGCGCCGCTTCTGGCTTTACGTTTTCCCG |
| | TRIM21₁₋₂₇₇ | TTATGCAATGTCATGGTACAGGCAGGCCCCCGGCAAAGAAAGGGAA |
| | | AGTGTGGCCAGAATCTCTAGTGGTGGCGGAACAATATATTATGCAG |
| | | ATTCCGTAAAAGGAAGATTTACGATTTCTCGTGAGGATGCTAAGAA |
| | | TACAGTGTATTTGCAGATGAACTCATTAAAGCCTGAAGATACTGCC |
| | | GTGTATTATTGCTACGTAGGAGGTTTTTGGGGCCAAGGTACTCAGG |
| | | TGACTGTCTCCTCTGGTGGTGGAGGCTCAGCTTCTGCCGCCCGTCT |
| | | AACAATGATGTGGGAGGAAGTGACGTGTCCAATCTGCTTGGACCCA |
| | | TTTGTGGAGCCAGTCTCAATAGAATGTGGACATTCATTCTGTCAGG |
| | | AATGCATCAGTCAAGTTGGAAAGGGCGGAGGATCAGTCTGCCCCGT |
| | | CTGTAGACAAAGATTCCTGTTAAAGAACCTAAGGCCAAACCGTCAG |
| | | CTGGCAAACATGGTAAACAACCTAAAGGAGATTTCACAGGAAGCTC |
| | | GTGAAGGAACTCAGGGCGAAAGATGCGCCGTCCATGGAGAAAGATT |
| | | ACACCTGTTCTGCGAGAAGGACGGCAAGGCTTTGTGTTGGGTCTGT |
| | | GCCCAATCCCGTAAACACAGAGACCACGCTATGGTCCCTCTGGAGG |
| | | AGGCCGCACAAGAATACCAAGAAAAGTTACAAGTGGCACTGGGAGA |
| | | ACTGCGTAGAAAACAAGAACTAGCAGAGAAATTGGAGGTCGAAATA |
| | | GCTATTAAAAGAGCCGACTGGAAGAAGACCGTGGAAACTCAAAAGT |
| | | CTAGGATCCATGCAGAGTTTGTACAACAGAAAAACTTCTTAGTAGA |
| | | AGAGGAGCAGAGACAATTACAAGAGCTTGAGAAGGATGAGAGGGAG |
| | | CAGCTTCGTATATTAGGAGAAAAGGAGGCAAAGTTGGCCCAGCAAT |
| | | CTCAGGCTTTACAGGAATTAATTTCAGAGTTAGATAGGAGATGTCA |
| | | TTCCTCAGCACTAGAGTTAGTTCAGGAGGTAATCATAGTTTTGGAA |
| | | AGGTCTGAGTCATGGAACTTAAAGGATCTTGATATTAGGAGTCCAG |
| | | AATTACGTAGTGTTTGCCACGTACCCGGCTGT |
| 382 | TRIM21₁₋₂₇₇- | ATGGCCTCCGCAGCCAGATTAACGATGATGTGGGAAGAGGTTACAT |
| | ASC^{VHH}_ | GTCCCATCTGCTTAGATCCTTTCGTCGAACCTGTCTCAATTGAGTG |
| | Cys | CGGACACTCATTTTGCCAAGAATGTATCTCACAAGTCGGAAAAGGA |
| | | GGCGGTTCTGTGTGCCCAGTTTGTCGTCAAAGATTTCTGTTGAAAA |
| | | ATCTGAGACCAAACAGACAACTGGCTAACATGGTCAACAATTTAAA |
| | | GGAAATATCCCAAGAAGCTCGTGAAGGAACCCAGGGCGAGCGTTGT |
| | | GCCGTACATGGCGAAAGATTAGATTTATTCTGTGAAAAGGATGGAA |
| | | AAGCTTTATGTTGGGTCTGTGCACAATCCAGAAAGCACAGGGATCA |
| | | TGCAATGGTCCCCCTTGAAGAGGCAGCACAGGAATACCAGGAAAAG |
| | | TTGCAAGTAGCACTGGGAGAACTTAGGCGTAAGCAGGAGTTGGCTG |
| | | AGAAACTAGAAGTCGAGATTGCTATTAAACGTGCCGACTGGAAGAA |
| | | GACGGTCGAAACTCAGAAATCTAGGATACATGCTGAGTTCGTTCAA |
| | | CAAAAAAATTTTCTGGTCGAAGAGGAGCAAAGGCAGTTGCAGGAAC |
| | | TGGAAAAAGATGAACGTGAACAATTAAGAATCTTGGGAGAGAAGGA |
| | | AGCAAAACTTGCCCAGCAATCTCAAGCACTTCAGGAGCTAATTAGT |
| | | GAACTAGACAGGAGGTGCCATAGTTCCGCCCTTGAACTATTGCAAG |
| | | AAGTAATTATCGTACTAGAGCGTTCCGAGTCTTGGAATCTAAAAGA |
| | | CTTGGATATCACCTCCCCCGAATTACGTTCAGTTTGTCACGTTCCC |
| | | GGTGGTGGAGGAGGCTCCCAATTACAGTTAGTCGAATCTGGCGGCG |
| | | GATTGGTCCAGCCTGGAGGATCACTAAAGCTGAGTTGTGCCGCATC |
| | | AGGATTTACGTTCAGTAGATACGCTATGAGTTGGTACAGACAGGCT |
| | | CCAGGAAAAGAGAGGGAGTCTGTAGCCAGGATCTCCTCTGGTGGAG |
| | | GAACGATTTATTATGCTGATTCTGTAAAGGGAAGATTTACAATCAG |
| | | TCGTGAAGATGCCAAGAACACAGTATATTTACAAATGAATTCCCTG |
| | | AAGCCAGAAGATACGGCCGTTTATTACTGTTATGTAGGAGGTTTTT |
| | | GGGGTCAGGGTACGCAAGTAACAGTTTCTTCTTGT |
| 383 | VHL₁₅₂₋₂₁₃- | ACTTTGCCCGTATATACGCTAAAGGAAAGGTGTCTACAGGTCGTGA |
| | ASC^{VHH}_ | GGTGAGTTGTGAAGCCAGAGAACTATAGACGTTTGGACATAGTAAG |
| | TRIM21₁₋₂₇₇ | GTCCCTATACGAGGATTTGGAAGATCATCCTAACGTCCAGAAAGAC |
| | -Cys | CTGGAAAGGCTAACCCAGGAAAGAATAGCCCATCAGAGGATGGGCG |
| | | ATGGAGGTGGCGGCTCTCAGTTACAGCTGGTGGAATCCGGCGGTGG |
| | | TTTGGTACAACCCGGCGGCAGTCTTAAGTTATCATGTGCCGCTTCC |
| | | GGTTTCACCTTGAGTAGATACGCTATGAGTTGGTAGAGACAGGCTC |
| | | CCGGAAAAGAAAGGGAGTCCGTAGCACGTATCAGTTCAGGAGGAGG |
| | | TACGATCTACTACGCAGACTCAGTCAAGGGCAGGTTCACGATTTCA |
| | | AGGGAGGACGCCAAGAATACCGTTTACCTGCAGATGAACTCTTTAA |
| | | AGCCTGAAGACACTGCCGTGTACTACTGCTATGTTGGCGGCTTCTG |
| | | GGGTCAGGGCACGCAAGTAACTGTCTCCTCCGGAGGTGGCGGTTCA |
| | | GCCTCTGCTGCCCGTCTGACTATGATGTGGGAAGAAGTGACCTGCC |
| | | CCATTTGTTTGGACCCTTTTGTCGAACCTGTTTCAATTGAGTGTGG |
| | | ACACTCTTTTTGTCAAGAGTGTATCAGTCAGGTGGGCAAAGGCGGA |
| | | GGATCTGTATGCCCTGTGTGCCGTCAGAGATTCCTATTGAAGAACT |
| | | TGCGTCCAAATCGTCAATTAGCAAACATGGTGAATAACCTAAAAGA |
| | | AATCTCCCAAGAGGCCAGAGAAGGCACGCAAGGAGAAAGATGTGCA |
| | | GTCCATGGCGAGAGACTACATTTATTTTGTGAAAAGGACGGCAAAG |
| | | CCCTATGTTGGGTTTGCGCTCAGTCCAGGAAGCATCGTGATCACGC |
| | | AATGGTTCCATTAGAGGAAGCTGCCCAGGAATATCAGGAAAAACTA |
| | | CAGGTTGCTCTTGGCGAACTGAGAAGAAAGCAAGAACTGGCAGAGA |
| | | AATTGGAGGTGGAAATTGCTATCAAACGTGCAGATTGGAAAAAAAC |
| | | CGTGGAGACTCAGAAGTCCAGGATACATGCAGAATTTGTTCAGCAG |
| | | AAGAATTTTCTAGTAGAAGAGGAACAGAGACAGCTTCAAGAACTAG |
| | | AGAAGGATGAACGTGAGCAGTTGAGAATTTTAGGAGAAAAGGAAGC |
| | | AAAGTTAGCCCAACAGAGTCAGGCACTACAAGAGCTTATCTCAGAG |
| | | CTTGACCGTAGATGCCATTCTAGTGCTCTAGAGCTACTGCAGGAGG |
| | | TGATAATCGTCTTAGAAAGGAGTGAAAGTTGGAACTTGAAAGACTT |
| | | GGACATCACTTCACCCGAGTTACGTAGTGTTTGTCATGTCCCCGGC |
| | | TGT |
| 384 | K19- | GACCTGGGAAAAAAGCTGCTGGAAGCCGCTAGAGCCGGCCAGGACG |
| | hFc(N297A) | ACGAGGTTAGAATCCTGATGGCCAACGGGGCCGACGTGAACGCCAG |
| | -Cys | CGACAGATGGGGCTGGACCCCTCTGCACCTGGCTGCTTGGTGGGGA |
| | | CATCTGGAAATCGTGGAAGTGCTGCTGAAGCGGGGCGCTGATGTTT |
| | | CCGCCGCCGATCTCCACGGCCAGAGCCCCCTGCATCTGGCCGCTAT |
| | | GGTGGGCCACCTGGAAATCGTGGAAGTCCTGCTGAAGTACGGCGCA |
| | | GATGTGAACGCCAAAGATACCATGGGCGCTACACCCCTGCACCTGG |
| | | CCGCCAGAAGCGGCCACCTGGAAATTGTGGAAGAGCTGCTTAAAAA |
| | | CGGCGCCGACATGAACGCCCAGGACAAGTTCGGCAAGACAACCTTC |
| | | GACATCAGCACCGACAACGGCAATGAGGACCTGGCCGAGATCCTCC |
| | | AGAAACTGGGCGGCGGAGGCAGCGACAAGACCCACACCTGCCCTCC |
| | | ATGCCCCGCCCCTGAGCTGCTGGGCGGACCTTCCGTGTTCCTGTTT |
| | | CCCCCTAAGCCCAAGGACACCCTGATGATCTCTCGGACCCCAGAGG |
| | | TGACCTGTGTGGTGGTGGACGTGTCCCACGAGGATCCTGAGGTTAA |
| | | GTTCAACTGGTATGTGGACGGCGTGGAAGTGCACAATGCCAAGACC |
| | | AAGCCTCGGGAAGAGCAGTACGCCAGTACATACAGAGTGGTCAGCG |
| | | TGCTCACCGTGCTGCACCAGGATTGGCTGAACGGCAAAGAGTACAA |
| | | GTGCAAGGTGTCTAACAAGGCCCTGCCAGCCCCTATCGAGAAGACC |
| | | ATCAGCAAGGCCAAGGGCCAACCTAGAGAGCCCCAGGTGTACACCC |
| | | TGCCACCTAGCAGAGAGGAAATGACCAAGAACCAGGTGAGCCTGAC |
| | | CTGTCTGGTGAAGGGCTTCTACCCCAGCGACATCGCCGTGGAGTGG |
| | | GAGTCTAATGGACAACCTGAGAACAACTACAAAACAACACCTCCTG |
| | | TGCTGGACAGCGATGGCTCTTTTTTCCTGTACAGCAAACTGACAGT |
| | | GGATAAGTCCCGCTGGCAGCAGGGCAACGTGTTCAGCTGCAGCGTG |
| | | ATGCACGAGGCCCTGCACAATCACTACACCCAGAAGAGCCTGAGCC |
| | | TGTCTCCTGGCTGC |
| 385 | K19-Fc_{S239C} | GACCTGGGAAAAAAGCTGCTGGAAGCCGCTAGAGCCGGCCAGGACG |
| | | ACGAGGTTAGAATCCTGATGGCCAACGGGGCCGACGTGAACGCCAG |
| | | CGACAGATGGGGCTGGACCCCTCTGCACCTGGCTGCTTGGTGGGGA |
| | | CATCTGGAAATCGTGGAAGTGCTGCTGAAGCGGGGCGCTGATGTTT |
| | | CCGCCGCCGATCTCCACGGCCAGAGCCCCCTGCATCTGGCCGCTAT |
| | | GGTGGGCCACCTGGAAATCGTGGAAGTCCTGCTGAAGTACGGCGCA |
| | | GATGTGAACGCCAAAGATACCATGGGCGCTACACCCCTGCACCTGG |
| | | CCGCCAGAAGCGGCCACCTGGAAATTGTGGAAGAGCTGCTTAAAAA |
| | | CGGCGCCGACATGAACGCCCAGGACAAGTTCGGCAAGACAACCTTC |
| | | GACATCAGCACCGACAACGGCAATGAGGACCTGGCCGAGATCCTCC |
| | | AGAAACTGGGCGGCGGAGGCAGCGACAAGACCCACACCTGCCCTCC |
| | | ATGCCCCGCCCCTGAGCTGCTGGGCGGACCTTGCGTGTTCCTGTTT |
| | | CCCCCTAAGCCCAAGGACACCCTGATGATCTCTCGGACCCCAGAGG |
| | | TGACCTGTGTGGTGGTGGACGTGTCCCACGAGGATCCTGAGGTTAA |
| | | GTTCAACTGGTATGTGGACGGCGTGGAAGTGCACAATGCCAAGACC |
| | | AAGCCTCGGGAAGAGCAGTACGCCAGTACATACAGAGTGGTCAGCG |
| | | TGCTCACCGTGCTGCACCAGGATTGGCTGAACGGCAAAGAGTACAA |
| | | GTGCAAGGTGTCTAACAAGGCCCTGCCAGCCCCTATCGAGAAGACC |
| | | ATCAGCAAGGCCAAGGGCCAACCTAGAGAGCCCCAGGTGTACACCC |
| | | TGCCACCTAGCAGAGAGGAAATGACCAAGAACCAGGTCAGCCTGAC |
| | | CTGTCTGGTGAAGGGCTTCTACCCCAGCGACATCGCCGTGGAGTGG |
| | | GAGTCTAATGGACAACCTGAGAACAACTAGAAAACAACACCTCCTG |
| | | TGCTGGACAGCGATGGCTCTTTTTTCCTGTACAGCAAACTGACAGT |
| | | GGATAAGTCCCGCTGGCAGCAGGGCAACGTGTTCAGCTGCAGCGTG |
| | | ATGCACGAGGCCCTGCACAATCACTACACCCAGAAGAGCCTGAGCC |
| | | TGTCTCCTGGCAAG |
| 386 | VHHCARD | CAACTGCAGCTGGTTGAATCTGGCGGCGGACTGGTTCAACCTGGCG |
| | -Fc | GATCTCTGAAGCTGAGCTGTGCCGCCAGCGGCTTCACCTTTAGCAG |
| | | ATACGCCATGAGCTGGTACAGACAGGCCCCTGGCAAAGAAAGGGAA |
| | | AGCGTGGCCAGAATCAGCAGCGGCGGAGGCACAATCTACTACGCCG |
| | | ATAGCGTGAAGGGCAGATTCACCATCAGCAGAGAGGACGCCAAGAA |
| | | CACCGTGTACCTGCAGATGAACAGCCTGAAGCCTGAGGACACCGCC |
| | | GTGTACTACTGTTACGTCGGCGGCTTTTGGGGCCAGGGCACACAAG |
| | | TGACAGTTAGCTCTGGTGGCGGAGGCTCCGACAAGACCCATACCTG |
| | | TCCTCCATGTCCTGCTCCAGAGCTGCTCGGAGGCCCTTCCGTGTTT |
| | | CTGTTCCCTCCAAAGCCAAAGGACACCCTGATGATCAGCAGAACCC |
| | | CTGAAGTGACCTGCGTGGTGGTGGATGTGTCCCACGAGGATCCCGA |
| | | AGTGAAGTTCAATTGGTACGTGGACGGCGTGGAAGTGCACAACGCC |
| | | AAGACCAAGCCTAGAGAGGAACAGTACAACAGCACCTACAGAGTGG |
| | | TGTCCGTGCTGACCGTGCTGCACCAGGATTGGCTGAACGGCAAAGA |
| | | GTACAAGTGCAAGGTGTCCAACAAGGCCCTGCCTGCTCCTATCGAG |
| | | AAAACCATCTCCAAGGCCAAGGGCCAGCCTAGGGAACCCCAGGTTT |
| | | ACACACTGCCTCCAAGCCGGGAAGAGATGACCAAGAACCAGGTGTC |
| | | CCTGACCTGCCTGGTCAAGGGCTTCTACCCTTCCGATATCGCCGTG |
| | | GAATGGGAGAGCAATGGCCAGCCTGAGAACAACTACAAGACCACAC |
| | | CTCCTGTGCTGGACAGCGACGGCTCATTCTTCCTGTACAGCAAGCT |
| | | GACAGTGGACAAGAGCAGATGGCAGCAGGGCAACGTGTTCAGCTGC |
| | | AGCGTGATGCACGAGGCCCTGCACAACCACTACACCCAGAAGTCCC |
| | | TGAGCCTGTCTCCTGGCAAA |
| 387 | VHH^{CARD}_ | CAACTGCAGCTGGTTGAATCTGGCGGCGGACTGGTTCAACCTGGCG |
| | Mb | GATCTCTGAAGCTGAGCTGTGCCGCCAGCGGCTTCACCTTTAGCAG |
| | | ATACGCCATGAGCTGGTACAGACAGGCCCCTGGCAAAGAAAGGGAA |
| | | AGCGTGGCCAGAATCAGCAGCGGCGGAGGCACAATCTACTACGCCG |
| | | ATAGCGTGAAGGGCAGATTCACCATCAGCAGAGAGGACGCCAAGAA |
| | | CACCGTGTACCTGCAGATGAACAGCCTGAAGCCTGAGGACACCGCC |
| | | GTGTACTACTGTTACGTCGGCGGCTTTTGGGGCCAGGGCACACAAG |
| | | TGACAGTTAGCTCTGGTGGCGGAGGCTCCGAACCTAAGTCTTCTGA |
| | | CAAGACCCATACCTGTCCTCCATGTCCTGGCCAGCCTAGGGAACCC |
| | | CAGGTTTACACACTGCCTCCAAGCCGGGAAGAGATGACCAAGAACC |
| | | AGGTGTCCCTGACCTGCCTGGTCAAGGGCTTCTACCCTTCCGATAT |
| | | CGCCGTGGAATGGGAGAGCAATGGCCAGCCTGAGAACAACTACAAG |
| | | ACCACACCTCCTGTGCTGGACAGCGACGGCTCATTCTTCCTGTACA |
| | | GCAAGCTGACAGTGGACAAGAGCAGATGGCAGCAGGGCAACGTGTT |
| | | CAGCTGCAGCGTGATGCACGAGGCCCTGCACAACCACTACACCCAG |
| | | AAGTCCCTGAGCCTGTCTCCTGGCAAA |
| 388 | VHL₅₄₋₂₁₃- | GATTACAAGGACGACGACGATAAGGGCAGCAGCGGCCACCACCATC |
| | VHH^{CARD} | ACCACCACGAGAACCTGTACTTCCAAGGCATGGAAGCCGGCAGACC |
| | | CAGACCTGTGCTGAGAAGCGTGAACAGCAGAGAACCGAGCCAAGTG |
| | | ATCTTCTGCAATCGGAGCCCCAGAGTGGTGCTGCCCGTGTGGCTTA |
| | | ATTTCGACGGCGAGCCCCAGCCTTATCCTACACTGCCACCTGGCAC |
| | | CGGCAGACGGATCCACTCTTATAGAGGACACCTGTGGCTGTTCCGC |
| | | GACGCCGGAACACATGATGGCCTGCTGGTCAATCAGACCGAGCTGT |
| | | TCGTGCCCAGCCTGAACGTTGACGGCCAGCCTATCTTCGCCAACAT |
| | | CACCCTGCCTGTGTACACCCTGAAAGAACGGTGCCTGCAGGTTGTG |
| | | CGGAGCCTGGTCAAGCCCGAGAATTACAGACGGCTGGACATCGTGC |
| | | GGTCCCTGTACGAGGATCTGGAAGATCACCCCAACGTGCAGAAGGA |
| | | CCTGGAACGGCTGACCCAAGAGAGAATCGCCCACCAGAGAATGGGA |
| | | GATGGCGGCGGAGGCGGAGAATTTGCTATGGCTCAGGTCCAGCTGC |
| | | AAGAGTCTGGCGGAGGACTTGTTCAGCCTGGCGGAAGCCTGAAGCT |
| | | GTCTTGTGCCGCCAGCGGCTTCACCTTCAGCAGATACGCCATGAGC |
| | | TGGTACAGACAGGCCCCTGGCAAAGAAAGGGAAAGCGTGGCCAGAA |
| | | TCAGCTCCGGCGGAGGAACAATCTACTACGCCGACAGCGTGAAGGG |
| | | CAGATTCACCATCTCCAGAGAGGAGGCCAAGAACACCGTGTACCTG |
| | | CAGATGAACTCCCTGAAGCCTGAGGACACCGCCGTGTACTACTGTT |
| | | ACGTCGGCGGCTTTTGGGGCCAGGGCACACAAGTGACAGTGTCTAG |
| | | TGGTGGCCAGCTCCAGCTGGTGGAAAGTGGTGGTGGACTGGTTCAA |
| | | CCAGGCGGCTCTCTGAAACTGAGCTGTGCCGCCTCTGGCTTCACAT |
| | | TCTCTCGCTACGCCATGTCTTGGTATCGGCAGGCTCCCGGAAAAGA |
| | | ACGCGAGTCTGTGGCCAGGATCTCTAGTGGCGGAGGCACCATCTAT |
| | | TATGCTGACTCCGTGAAAGGCCGGTTTACCATCAGCCGCGAGGATG |
| | | CCAAAAACACAGTCTATCTCCAAATGAACAGTCTGAAGCCCGAAGA |
| | | TACGGCCGTCTACTATTGCTATGTTGGCGGATTCTGGGGACAGGGA |
| | | ACCCAAGTCACTGTGTCCTCT |
| 389 | VHL- | GATTACAAGGACGACGACGATAAGGGCAGCAGCGGCCACCACCATC |
| | VHH^{CARD} | ACCACCACGAGAACCTGTACTTCCAAGGCATGCCCAGACGGGCCGA |
| | | GAACTGGGATGAAGCTGAAGTGGGAGCTGAGGAAGCCGGCGTCGAG |
| | | GAATATGGCCCTGAAGAAGATGGCGGCGAAGAGTCTGGCGCCGAGG |
| | | AAAGTGGACCTGAGGAAAGCGGTCCTGAAGAACTGGGCGCCGAAGA |
| | | GGAAATGGAAGCCGGAAGGCCTAGACCTGTGCTGCGGAGCGTGAAC |
| | | AGCAGAGAACCCAGCCAAGTGATCTTCTGCAATCGGAGCCCCAGAG |
| | | TGGTGCTGCCCGTGTGGCTTAATTTCGACGGCGAGCCCCAGCCTTA |
| | | TCCTACACTGCCACCTGGCACCGGCAGACGGATCCACTCTTATAGA |
| | | GGACACCTGTGGCTGTTCAGAGATGCCGGCACACACGATGGCCTGC |
| | | TGGTCAATCAGACCGAGCTGTTCGTGCCCAGCCTGAACGTTGACGG |
| | | CCAGCCTATCTTCGCCAACATCACCCTGCCTGTGTACACCCTGAAA |
| | | GAACGGTGCCTGCAGGTTGTGCGGAGCCTGGTCAAGCCCGAGAATT |
| | | ACAGACGGCTGGACATCGTGCGGTCCCTGTACGAGGATCTGGAAGA |
| | | TCACCCCAACGTGCAGAAGGACCTGGAACGGCTGACCCAAGAGAGA |
| | | ATCGCCCACCAGAGAATGGGAGATGGCGGAGGTGGCGGAGAGTTTG |
| | | CTATGGCTCAGGTGCAGCTCCAAGAAAGCGGCGGAGGACTTGTTCA |
| | | GCCTGGCGGATCTCTGAAGCTGAGCTGTGCCGCCAGCGGCTTCACC |
| | | TTTAGCAGATACGCCATGAGCTGGTACAGACAGGCCCCTGGCAAAG |
| | | AAAGGGAAAGCGTGGCCAGAATCAGCAGCGGAGGCGGCACAATCTA |
| | | CTACGCCGATAGCGTGAAGGGCAGATTCACCATCAGCAGAGAGGAC |
| | | GCCAAGAACACCGTGTACCTGCAGATGAACAGCCTGAAGCCTGAGG |
| | | ACACCGCCGTGTACTACTGTTACGTCGGCGGCTTTTGGGGCCAGGG |
| | | CACACAAGTGACAGTGTCTAGCGGAGGACAGCTCCAGCTGGTGGAA |
| | | TCTGGTGGTGGACTGGTTCAACCAGGCGGCAGCCTGAAACTGTCTT |
| | | GTGCCGCTTCTGGCTTCACATTCTCCCGCTACGCCATGTCTTGGTA |
| | | TAGGCAGGCTCCCGGAAAAGAGAGAGAGTCCGTCGCCAGAATCTCC |
| | | TCTGGCGGCGGAACCATCTATTATGCCGACTCCGTGAAAGGCCGGT |
| | | TCACAATCTCCAGAGAAGATGCCAAAAACACGGTCTATCTCCAAAT |
| | | GAACTCACTGAAGCCCGAAGATACGGCCGTCTACTATTGCTATGTT |
| | | GGCGGATTCTGGGGACAGGGAACCCAAGTCACTGTGTCCTCT |
| 390 | VHL- | ATGGACTACAAGGACGATGACGATAAAGGCAGCAGCGGTCACCACC |
| | VHH^{CARD} | ACCACCACCACGAAAACCTGTATTTCCAGGGTATGGAAGCGGGTCG |
| | | TCCGCGTCCGGTGCTGCGTAGCGTTAACAGCCGTGAGCCGAGCCAA |
| | | GTGATCTTCTGCAACCGTAGCCCGCGTGTGGTTCTGCCGGTTTGGC |
| | | TGAACTTTGATGGTGAACCGCAACCGTACCCGACCCTGCCGCCGGG |
| | | TACCGGTCGTCGTATTCACAGCTATCGTGGTCATCTGTGGCTGTTC |
| | | CGTGATGCGGGTACCCACGATGGTCTGCTGGTGAACCAAACCGAGC |
| | | TGTTCGTGCCGAGCCTGAACGTTGACGGTCAGCCGATCTTTGCGAA |
| | | CATTACCCTGCCGGTTTACACCCTGAAGGAACGTTGCCTGCAAGTG |
| | | GTTCGTAGCCTGGTGAAACCGGAGAACTACCGTCGTCTGGATATCG |
| | | TTCGTAGCCTGTATGAAGACCTGGAGGATCACCCGAACGTGCAGAA |
| | | GGACCTGGAACGTCTGACCCAAGAGCGTATTGCGCACCAGCGTATG |
| | | GGTGATGGTGGCGGTGGCGGTGAATTTGCGATGGCGCAGGTGCAAC |
| | | TGCAAGAAAGCGGCGGTGGCCTGGTTCAACCGGGTGGCAGCCTGAA |
| | | GCTGAGCTGCGCGGCGAGCGGTTTCACCTTTAGCCGTTACGCGATG |
| | | AGCTGGTATCGTCAGGCGCCGGGCAAAGAACGTGAGAGCGTTGCGC |
| | | GTATCAGCAGCGGTGGCGGTACCATTTACTATGCGGACAGCGTGAA |
| | | GGGTCGTTTCACCATCAGCCGTGAAGATGCGAAAAACACCGTTTAC |
| | | CTGCAAATGAACAGCCTGAAGCCGGAGGACACCGCGGTGTACTATT |
| | | GCTATGTTGGCGGTTTTTGGGGCCAAGGTACCCAGGTGACCGTTAG |
| | | CAGCGGCGGTCAACTGCAGCTGGTTGAGAGCGGCGGTGGCCTGGTG |
| | | CAGCCGGGTGGCAGCCTGAAGCTGTCTTGCGCGGCGAGCGGCTTTA |
| | | CCTTTAGCCGTTATGCGATGAGCTGGTACCGCCAAGCGCCGGGTAA |
| | | AGAACGTGAGAGCGTGGCGCGCATCAGCAGCGGTGGCGGTACGATC |
| | | TATTATGCGGACAGCGTGAAAGGTCGTTTCACCATTAGCCGTGAAG |
| | | ATGCGAAGAACACCGTTTACCTGCAGATGAACAGCCTGAAACCGGA |
| | | GGATACCGCGGTTTATTACTGCTACGTTGGCGGTTTTTGGGGTCAG |
| | | GGCACCCAAGTTACCGTTAGCAGC |
| 391 | Cys- | TGCCAGTTACAACTTGTCGAGTCAGGCGGTGGCTTGGTACAGCCCG |
| | ASC(VHH) | GTGGCTCTTTAAAACTTAGTTGTGCTGCCTCAGGATTTACGTTCTC |
| | -Mb-linker- | TAGGTACGCCATGTCATGGTATAGGCAGGCCCCCGGCAAGGAAAGG |
| | VHL₁₅₂₋ | GAGTCAGTTGCTAGGATTAGTTCCGGCGGAGGAACTATCTATTATG |
| | | CAGACTCTGTAAAGGGCCGTTTCACCATCTCTCGTGAGGATGCCAA |
| | | AAACACTGTGTACCTACAGATGAATAGTTTGAAACCTGAGGATACA |
| | | GCTGTATATTATTGTTATGTCGGTGGTTTTTGGGGCCAGGGCACGC |
| | | AAGTCACCGTGTCCTCTGAGCCCAAAAGTAGTGATAAAACTCACAC |
| | | GTGTCCACCCTGCCCTGGACAACCAAGAGAGCCCCAGGTATATACT |
| | | CTACCACCATCACGTGAAGAGATGACCAAGAACCAAGTATCCCTTA |
| | | CCTGTTTGGTTAAAGGCTTCTATCCAAGTGATATTGCCGTGGAATG |
| | | GGAGAGTAATGGTCAACCCGAAAACAATTACAAGACCACCCCTCCT |
| | | GTCCTGGACTCCGACGGAAGTTTTTTTCTATACAGTAAACTTACTG |
| | | TGGATAAGAGTCGTTGGCAACAAGGAAACGTGTTCTCCTGCTCAGT |
| | | GATGCACGAAGCATTACACAACCATTATACGCAGAAATCTCTATCT |
| | | CTTAGTCCTGGCGGCGGTGGATCTACTTTGCCAGTCTATACCCTAA |
| | | AAGAACGTTGTCTGCAAGTGGTGAGGAGTTTAGTCAAGCCTGAAAA |
| | | CTACCGTCGTTTGGACATAGTAAGGTCACTATATGAAGACTTGGAA |
| | | GATCACCCCAACGTGCAAAAAGATCTAGAAAGACTGAGGGAGGAGA |
| | | GAATCGCACACCAGAGGATGGGTGAT |

Examples of some polypeptide sequences and polynucleotides encoding sequences thereof for exemplary degradation constructs described herein are provided below in Table 20. Construct details are written N to C and 5′ to 3′. Although no linker is not explicitly included in the construct details, a linker may be present a person of ordinary skill in the art would be able to determine the linker via methods known in the art. Additionally, N terminal and or C terminal protein tags may be present and a person or ordinary skill in the art would be able to determine the protein tag sequence via methods known in the art.

TABLE 20
Examples of Some Polynucleotides Encoding Degradation Constructs.

| | SEQ | | | |
|---|---|---|---|---|
| | ID | | SEQ | |
| Name | NO: | Sequence | ID NO: | Sequence |
| HRAS^{VHH}- | 392 | MHHHHHHENLYFQGEV | 402 | ATGCATCACCACCACCACCACGAGAACCT |
| cys | | QLLESGGGLVQPGGSL | | GTACTTCCAGGGCGAGGTGCAACTGCTGG |
| | | RLSCAASGFTFSTFSM | | AGAGCGGTGGCGGTCTGGTTCAACCGGGC |
| | | NWVRQAPGKGLEWVSY | | GGTAGCCTGCGTCTGAGCTGCGCGGCGAG |
| | | ISRTSKTIYYADSVKG | | CGGTTTCACCTTTAGCACCTTTAGCATGA |
| | | RFTISRDNSKNTLYLQ | | ACTGGGTGCGTCAAGCGCCGGGCAAGGGT |
| | | MNSLRAEDTAVYYCAR | | CTGGAGTGGGTTAGCTATATCAGCCGTAC |
| | | GRFFDYWGQGTLVTVS | | CAGCAAGACCATTTACTATGCGGACAGCG |
| | | SC | | TGAAAGGCCGTTTCACCATCAGCCGTGAT |
| | | | | AACAGCAAAAACACCCTGTACCTGCAGAT |
| | | | | GAACAGCCTGCGTGCGGAAGACACCGCGG |
| | | | | TTTACTATTGCGCGCGTGGTCGTTTCTTT |
| | | | | GATTATTGGGGCCAAGGTACCCTGGTGAC |
| | | | | CGTTAGCAGCTGCTAATGAAAGCTT |
| HRAS^{VHH}- | 393 | MHHHHHHENLYFQGEV | 403 | ATGCATCACCACCACCACCACGAGAACCT |
| cys | | QLLESGGGLVQPGGSL | | GTACTTCCAGGGCGAGGTGCAACTGCTGG |
| | | RLSCAASGFTFSTFSM | | AGAGCGGTGGCGGTCTGGTTCAACCGGGC |
| | | NWVRQAPGKGLEWVSY | | GGTAGCCTGCGTCTGAGCTGCGCGGCGAG |
| | | ISRTSKTIYYADSVKG | | CGGTTTCACCTTTAGCACCTTTAGCATGA |
| | | RFTISRDNSKNTLYLQ | | ACTGGGTGCGTCAAGCGCCGGGCAAGGGT |
| | | MNSLRAEDTAVYYCAR | | CTGGAGTGGGTTAGCTATATCAGCCGTAC |
| | | GRFFDYWGQGTLVTVS | | CAGCAAGACCATTTACTATGCGGACAGCG |
| | | S | | TGAAAGGCCGTTTCACCATCAGCCGTGAT |
| | | | | AACAGCAAAAACACCCTGTACCTGCAGAT |
| | | | | GAACAGCCTGCGTGCGGAAGACACCGCGG |
| | | | | TTTACTATTGCGCGCGTGGTCGTTTCTTT |
| | | | | GATTATTGGGGCCAAGGTACCCTGGTGAC |
| | | | | CGTTAGCAGCTAATGAAAGCTT |
| K19- | 394 | MHHHHHHENLYFQGDL | 404 | ATGCATCACCACCACCACCACGAGAACCT |
| cys | | GKKLLEAARAGQDDEV | | GTACTTCCAGGGTGACCTGGGCAAGAAAC |
| | | RILMANGADVNASDRW | | TGCTGGAGGCGGCGCGTGCGGGTCAAGAC |
| | | GWTPLHLAAWWGHLEI | | GATGAAGTGCGTATCCTGATGGCGAACGG |
| | | VEVLLKRGADVSAADL | | TGCGGACGTTAACGCGAGCGATCGTTGGG |
| | | HGQSPLHLAAMVGHLE | | GCTGGACCCCGCTGCACCTGGCGGCGTGG |
| | | IVEVLLKYGADVNAKD | | TGGGGTCACCTGGAGATTGTGGAAGTTCT |
| | | TMGATPLHLAARSGHL | | GCTGAAACGTGGTGCGGATGTGAGCGCGG |
| | | EIVEELLKNGADMNAQ | | CGGATCTGCACGGCCAGAGCCCGCTGCAT |
| | | DKFGKTTFDISTDNGN | | CTGGCGGCGATGGTGGGTCACCTGGAGAT |
| | | EDLAEILQKLC | | CGTGGAAGTTCTGCTGAAGTATGGCGCGG |
| | | | | ATGTTAACGCGAAAGATACGATGGGTGCG |
| | | | | ACCCCGCTGCATTTAGCTGCGCGTAGCGG |
| | | | | CCACCTGGAAATTGTTGAGGAACTGCTGA |
| | | | | AGAACGGTGCGGACATGAACGCGCAGGAT |
| | | | | AAGTTCGGCAAAACCACCTTTGACATCAG |
| | | | | CACCGATAACGGTAACGAGGATCTGGCGG |
| | | | | AAATTCTGCAAAAACTGTGCTAATGAAAG |
| | | | | CTT |
| k19 | 395 | MHHHHHHENLYFQGDL | 405 | ATGCATCACCACCACCACCACGAGAACCT |
| | | GKKLLEAARAGQDDEV | | GTACTTCCAGGGTGACCTGGGCAAGAAAC |
| | | RILMANGADVNASDRW | | TGCTGGAGGCGGCGCGTGCGGGTCAAGAC |
| | | GWTPLHLAAWWGHLEI | | GATGAAGTGCGTATCCTGATGGCGAACGG |
| | | VEVLLKRGADVSAADL | | TGCGGACGTTAACGCGAGCGATCGTTGGG |
| | | HGQSPLHLAAMVGHLE | | GCTGGACCCCGCTGCACCTGGCGGCGTGG |
| | | IVEVLLKYGADVNAKD | | TGGGGTCACCTGGAGATTGTGGAAGTTCT |
| | | TMGATPLHLAARSGHL | | GCTGAAACGTGGTGCGGATGTGAGCGCGG |
| | | EIVEELLKNGADMNAQ | | CGGATCTGCACGGCCAGAGCCCGCTGCAT |
| | | DKFGKTTFDISTDNGN | | CTGGCGGCGATGGTGGGTCACCTGGAGAT |
| | | EDLAEILQKL | | CGTGGAAGTTCTGCTGAAGTATGGCGCGG |
| | | | | ATGTTAACGCGAAAGATACGATGGGTGCG |
| | | | | ACCCCGCTGCATTTAGCTGCGCGTAGCGG |
| | | | | CCACCTGGAAATTGTTGAGGAACTGCTGA |
| | | | | AGAACGGTGCGGACATGAACGCGCAGGAT |
| | | | | AAGTTCGGCAAAACCACCTTTGACATCAG |
| | | | | CACCGATAACGGTAACGAGGATCTGGCGG |
| | | | | AAATTCTGCAAAAACTGTAATGAAAGCTT |
| KRAS^{VHH}- | 396 | MHHHHHHENLYFQGEV | 406 | ATGCATCACCACCACCACCACGAGAACCT |
| Cys | | QLVESGGGSVQTGGSL | | GTACTTCCAGGGCGAGGTGCAACTGGTTG |
| | | RLSCAVSGNIGSSYCM | | AAAGCGGTGGCGGTAGCGTGCAGACCGGC |
| | | GWFRQAPGKKREAVAR | | GGTAGCCTGCGTCTGAGCTGCGCGGTTAG |
| | | IVRDGATGYADYVKGR | | CGGCAACATCGGTAGCAGCTATTGCATGG |
| | | FTISRDSAKNTLYLQM | | GCTGGTTCCGTCAAGCGCCGGGTAAGAAA |
| | | NRLIPEDTAIYYCAAD | | CGTGAAGCGGTTGCGCGTATTGTTCGTGA |
| | | LPPGCLTQAIWNFGYR | | TGGTGCGACCGGTTACGCGGATTATGTGA |
| | | GQGTLVTVSSC | | AGGGTCGTTTTACCATTAGCCGTGACAGC |
| | | | | GCGAAAAACACCCTGTACCTGCAGATGAA |
| | | | | CCGTCTGATCCCGGAAGACACCGCGATTT |
| | | | | ACTATTGCGCGGCGGATCTGCCGCCGGGT |
| | | | | TGCCTGACCCAGGCGATTTGGAACTTTGG |
| | | | | TTATCGTGGCCAAGGTACCCTGGTGACCG |
| | | | | TTAGCAGCTGC |
| KRAS^{VHH}- | 397 | MHHHHHHENLYFQGEV | 407 | ATGCATCACCACCACCACCACGAGAACCT |
| Cys | | QLVESGGGSVQTGGSL | | GTACTTCCAGGGCGAGGTGCAACTGGTTG |
| | | RLSCAVSGNIGSSYCM | | AAAGCGGTGGCGGTAGCGTGCAGACCGGC |
| | | GWFRQAPGKKREAVAR | | GGTAGCCTGCGTCTGAGCTGCGCGGTTAG |
| | | IVRDGATGYADYVKGR | | CGGCAACATCGGTAGCAGCTATTGCATGG |
| | | FTISRDSAKNTLYLQM | | GCTGGTTCCGTCAAGCGCCGGGTAAGAAA |
| | | NRLIPEDTAIYYCAAD | | CGTGAAGCGGTTGCGCGTATTGTTCGTGA |
| | | LPPGCLTQAIWNFGYR | | TGGTGCGACCGGTTACGCGGATTATGTGA |
| | | GQGTLVTVSS | | AGGGTCGTTTTACCATTAGCCGTGACAGC |
| | | | | GCGAAAAACACCCTGTACCTGCAGATGAA |
| | | | | CCGTCTGATCCCGGAAGACACCGCGATTT |
| | | | | ACTATTGCGCGGCGGATCTGCCGCCGGGT |
| | | | | TGCCTGACCCAGGCGATTTGGAACTTTGG |
| | | | | TTATCGTGGCCAAGGTACCCTGGTGACCG |
| | | | | TTAGCAGCTAATGAAAGCTT |
| BC2- | 76 | QVQLVESGGGLVQPGG | 408 | GCACAAGTCCAATTAGTAGAAAGTGGAGGAG |
| cys | | SLTLSCTASGFTLDHY | | GTTTAGTTCAGCCAGGCGGCAGTCTGACTCT |
| | | DIGWFRQAPGKEREGV | | GAGTTGTACCGCCTCAGGCTTCACGCTAGAC |
| | | SCINNSDDDTYYADSV | | CATTATGACATAGGTTGGTTCAGACAGGCTC |
| | | KGRETIFMNNAKDTVY | | CAGGCAAAGAGCGTGAAGGAGTCTCTTGCAT |
| | | LQMNSLKPEDTAIYYC | | AAACAACTCCGACGACGACACATATTATGCC |
| | | AEARGCKRGRYEYDFW | | GACTCAGTGAAGGGCCGTTTCACTATATTTA |
| | | GQGTQVTVSSC | | TGAATAACGCCAAGGATACGGTTTACCTTCA |
| | | | | GATGAACAGTCTGAAGCCAGAAGATACTGCT |
| | | | | ATATATTACTGTGCTGAAGCCCGTGGTTGCA |
| | | | | AGCGTGGAAGATACGAATATGACTTTTGGGG |
| | | | | ACAAGGAACGCAAGTCACGGTCAGTTCTTGC |
| MOP3 | 398 | VSDVPRKLEVVAATPT | 409 | ATATGGTGAGCGACGTGCCGCGTAAACTGGA |
| | | SLLISWDAPAVTVRYY | | AGTGGTTGCGGCGACCCCGACCAGCCTGCTG |
| | | RITYGETGGNSPVQEF | | ATTAGCTGGGATGCGCCGGCGGTGACCGTGC |
| | | TVPGSKSTATISGLKP | | GTTACTATCGTATCACCTACGGCGAGACCGG |
| | | GVDYTITVYAVTSGGG | | TGGCAACAGCCCGGTGCAGGAATTCACCGTT |
| | | SFAEYWALLSGGGSPI | | CCGGGTAGCAAGAGCACCGCGACCATCAGCG |
| | | SINYRTALE | | GCCTGAAACCGGGTGTGGACTACACCATTAC |
| | | | | CGTGTATGCGGTTACCAGCGGTGGCGGTAGC |
| | | | | TTTGCGGAGTATTGGGCGCTGCTGAGCGGCG |
| | | | | GCGGTAGCCCGATTAGCATTAACTATCGCAC |
| | | | | CGCGCTGGAATAAGGATCC |
| MOP3+ | 137 | VSDVPRKLEVVAATPT | 410 | CATATGGTGAGCGACGTGCCGCGTAAACTGG |
| | | SLLISWDAPAVTVRYY | | AAGTGGTTGCGGCGACCCCGACCAGCCTGCT |
| | | RITYGETSGGGSFAEY | | GATTAGCTGGGATGCGCCGGCGGTGACCGTG |
| | | WALLSGGGSVQEFTVP | | CGTTACTATCGTATCACCTACGGCGAGACCA |
| | | GSKSTATISGLKPGVD | | GCGGTGGCGGTAGCTTCGCGGAATATTGGGC |
| | | YTITVYAVTSGGGSFA | | GCTGCTGAGCGGCGGTGGCAGCGTGCAGGAG |
| | | EYWALLSGGGSPISIN | | TTTACCGTTCCGGGTAGCAAGAGCACCGCGA |
| | | YRTALE | | CCATCAGCGGCCTGAAACCGGGTGTGGACTA |
| | | | | CACCATTACCGTGTATGCGGTTACCAGCGGT |
| | | | | GGCGGTAGCTTTGCGGAATACTGGGCGCTGC |
| | | | | TGAGCGGCGGCGGTAGCCCGATTAGCATTAA |
| | | | | CTATCGCACCGCGCTGGAATAAGGATCC |
| VHH^{CARD} | 399 | MHHHHHHENLYFQGQL | 411 | ATGCATGAGGAGGAGGAGCACGAGAACCTGT |
| with | | QLVESGGGLVQPGGSL | | ACTTCCAGGGTCAACTGCAGCTGGTTGAGAG |
| Cysteine | | KLSCAASGFTFSRYAM | | CGGTGGCGGTCTGGTTCAACCGGGCGGTAGC |
| | | SWYRQAPGKERESVAR | | CTGAAACTGAGCTGCGCGGCGAGCGGTTTCA |
| | | ISSGGGTIYYADSVKG | | CCTTTAGCCGTTACGCGATGAGCTGGTATCG |
| | | RFTISREDAKNTVYLQ | | TCAAGCGCCGGGCAAGGAGCGTGAAAGCGTG |
| | | MNSLKPEDTAVYYCYV | | GCGCGTATCAGCAGCGGCGGTGGCACCATTT |
| | | GGFWGQGTQVTVSSC | | ACTATGCGGACAGCGTGAAGGGCCGTTTCAC |
| | | | | CATCAGCCGTGAGGATGCGAAAAACACCGTT |
| | | | | TACCTGCAAATGAACAGCCTGAAGCCGGAAG |
| | | | | ACACCGCGGTGTACTATTGCTATGTTGGTGG |
| | | | | CTTTTGGGGTCAAGGCACCCAGGTGACCGTT |
| | | | | AGCAGCTGC |
| VHH^{CARD} | 400 | MHHHHHHENLYFQGQL | 412 | ATGCATCACCACCACCACCACGAGAACCTGT |
| | | QLVESGGGLVQPGGSL | | ACTTCCAGGGTCAACTGCAGCTGGTTGAGAG |
| | | KLSCAASGFTFSRYAM | | CGGTGGCGGTCTGGTTCAACCGGGCGGTAGC |
| | | SWYRQAPGKERESVAR | | CTGAAACTGAGCTGCGCGGCGAGCGGTTTCA |
| | | ISSGGGTIYYADSVKG | | CCTTTAGCCGTTACGCGATGAGCTGGTATCG |
| | | RFTISREDAKNTVYLQ | | TCAAGCGCCGGGCAAGGAGCGTGAAAGCGTG |
| | | MNSLKPEDTAVYYCYV | | GCGCGTATCAGCAGCGGCGGTGGCACCATTT |
| | | GGFWGQGTQVTVSS | | ACTATGCGGACAGCGTGAAGGGCCGTTTCAC |
| | | | | CATCAGCCGTGAGGATGCGAAAAACACCGTT |
| | | | | TACCTGCAAATGAACAGCCTGAAGCCGGAAG |
| | | | | ACACCGCGGTGTACTATTGCTATGTTGGTGG |
| | | | | CTTTTGGGGTCAAGGCACCCAGGTGACCGTT |
| | | | | AGCAGC |
| VHH^{CARD}- | 401 | MHHHHHHENLYFQ GQ | 413 | ATGCATCACCACCACCACCACGAGAACCTGT |
| LPETG | | LQLVESGGGLVQPGGS | | ACTTCCAGGGTCAACTGCAGCTGGTTGAGAG |
| | | LKLSCAASGFTFSRYA | | CGGTGGCGGTCTGGTTCAACCGGGCGGTAGC |
| | | MSWYRQAPGKERESVA | | CTGAAACTGAGCTGCGCGGCGAGCGGTTTCA |
| | | RISSGGGTIYYADSVK | | CCTTTAGCCGTTACGCGATGAGCTGGTATCG |
| | | GRETISREDAKNTVYL | | TCAAGCGCCGGGCAAGGAGCGTGAAAGCGTG |
| | | QMNSLKPEDTAVYYCY | | GCGCGTATCAGCAGCGGCGGTGGCACCATTT |
| | | VGGFWGQGTQVTVSSL | | ACTATGCGGACAGCGTGAAGGGTCGTTTCAC |
| | | PETG | | CATCAGCCGTGAGGATGCGAAAAACACCGTT |
| | | | | TACCTGCAAATGAACAGCCTGAAGCCGGAAG |
| | | | | ACACCGCGGTGTACTATTGCTATGTTGGTGG |
| | | | | CTTTTGGGGTCAAGGCACCCAGGTGACCGTT |
| | | | | AGCAGCCTGCCGGAGACCGGC |
| VHH^{CARD} | 83 | QLQLVESGGGLVQPGG | 414 | CAACTGCAGCTGGTTGAATCTGGCGGCGGAC |
| with C | | SLKLSCAASGFTFSRY | | TGGTTCAACCTGGCGGATCTCTGAAGCTGAG |
| | | AMSWYRQAPGKERESV | | CTGTGCCGCCAGCGGCTTCACCTTTAGCAGA |
| | | ARISSGGGTIYYADSV | | TACGCCATGAGCTGGTACAGACAGGCCCCTG |
| | | KGRFTISREDAKNTVY | | GCAAAGAAAGGGAAAGCGTGGCCAGAATCAG |
| | | LQMNSLKPEDTAVYYC | | CAGCGGCGGAGGCACAATCTACTACGCCGAT |
| | | YVGGFWGQGTQVTVSS | | AGCGTGAAGGGCAGATTCACCATCAGCAGAG |
| | | C | | AGGACGCCAAGAACACCGTGTACCTGCAGAT |
| | | | | GAACAGCCTGAAGCCTGAGGACACCGCCGTG |
| | | | | TACTACTGTTACGTCGGCGGCTTTTGGGGCC |
| | | | | AGGGCACACAAGTGACAGTGTCCTCTTGT |
| VHH^{CARD} | 82 | QLQLVESGGGLVQPGG | 415 | CAACTGCAGCTGGTTGAATCTGGCGGCGGAC |
| | | SLKLSCAASGFTFSRY | | TGGTTCAACCTGGCGGATCTCTGAAGCTGAG |
| | | AMSWYRQAPGKERESV | | CTGTGCCGCCAGCGGCTTCACCTTTAGCAGA |
| | | ARISSGGGTIYYADSV | | TACGCCATGAGCTGGTACAGACAGGCCCCTG |
| | | KGRFTISREDAKNTVY | | GCAAAGAAAGGGAAAGCGTGGCCAGAATCAG |
| | | LQMNSLKPEDTAVYYC | | CAGCGGCGGAGGCACAATCTACTACGCCGAT |
| | | YVGGFWGQGTQVTVSS | | AGCGTGAAGGGCAGATTCACCATCAGCAGAG |
| | | | | AGGACGCCAAGAACACCGTGTACCTGCAGAT |
| | | | | GAACAGCCTGAAGCCTGAGGACACCGCCGTG |
| | | | | TACTACTGTTACGTCGGCGGCTTTTGGGGCC |
| | | | | AGGGCACACAAGTGACAGTGTCCTCT |
| VHH^{CARD}- | 84 | QLQLVESGGGLVQPGG | 416 | CAACTGCAGCTGGTTGAATCTGGCGGCGGAC |
| LPETG | | SLKLSCAASGFTFSRY | | TGGTTCAACCTGGCGGATCTCTGAAGCTGAG |
| | | AMSWYRQAPGKERESV | | CTGTGCCGCCAGCGGCTTCACCTTTAGCAGA |
| | | ARISSGGGTIYYADSV | | TACGCCATGAGCTGGTACAGACAGGCCCCTG |
| | | KGRFTISREDAKNTVY | | GCAAAGAAAGGGAAAGCGTGGCCAGAATCAG |
| | | LQMNSLKPEDTAVYYC | | CAGCGGCGGAGGCACAATCTACTACGCCGAT |
| | | YVGGFWGQGTQVTVSS | | AGCGTGAAGGGCAGATTCACCATCAGCAGAG |
| | | LPETG | | AGGACGCCAAGAACACCGTGTACCTGCAGAT |
| | | | | GAACAGCCTGAAGCCTGAGGACACCGCCGTG |
| | | | | TACTACTGTTACGTCGGCGGCTTTTGGGGCC |
| | | | | AGGGCACACAAGTGACAGTGTCCAGCCTGCC |
| | | | | TGAGACAGGA |
| VHH^{CARD}- | 83 | QLQLVESGGGLVQPGG | 417 | CAACTACAGCTTGTTGAGTCTGGAGGCGGCC |
| Cys | | SLKLSCAASGFTFSRY | | TTGTCCAGCCTGGTGGCTCACTTAAGTTATC |
| | | AMSWYRQAPGKERESV | | ATGTGCTGCATCTGGATTTACCTTCTCCAGG |
| | | ARISSGGGTIYYADSV | | TATGCAATGTCTTGGTATAGGCAGGCACCTG |
| | | KGRFTISREDAKNTVY | | GTAAAGAAAGGGAATCCGTTGCAAGAATCAG |
| | | LQMNSLKPEDTAVYYC | | TTCAGGTGGCGGCACGATATACTACGCCGAC |
| | | YVGGFWGQGTQVTVSS | | TCAGTCAAAGGTCGTTTCACAATATCCAGGG |
| | | C | | AGGATGCCAAAAATACGGTTTATTTGCAAAT |
| | | | | GAATAGTTTGAAACCCGAAGATACAGCAGTG |
| | | | | TACTACTGCTACGTCGGAGGCTTCTGGGGAC |
| | | | | AAGGCACACAAGTCACCGTTTCCAGTTGC |

In some embodiments, the degradation construct has at least 80%, 85%, 90%, 91%, 92%, 9300, 940%, 950%, 9600, 9700 980%, or 9900 percent similarity and/or percent identity to any one of sequences of Table 20. In embodiments, the degradation construct has 80% to 1000%, 85% to 1000%, 90% to 1000%, 95% to 1000%, 97% to 1000%, 99% to 1000%, 90% to 99%, 95% to 99%, or 9700 to 990% percent similarity and/or percent identity to any one of sequences in Table 20.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed in particular embodiments, with the total length preferably being limited by the ease of preparation and use in the intended recombinant DNA protocol. Polynucleotides can be prepared, manipulated, and/or expressed using any of a variety of well-established techniques known and available in the art.

Promoters and Signal Sequences

In some embodiments, a vector may also comprise a sequence encoding a signal peptide (e.g., for nuclear localization, nucleolar localization, mitochondrial localization), fused to the polynucleotide encoding the modified looped protein. For example, a vector may comprise a nuclear localization sequence (e.g., from SV40 or cMyc) fused to the polynucleotide encoding the modified looped protein. Exemplary nuclear localization sequences are provided below:

(SEQ ID NO: 42)
SV40: PKKKRKV
(SEQ ID NO: 418)
NLP: AVKRPAATKKAGQAKKKKLD
(SEQ ID NO: 419)
TUS: KLKIKRPVK
(SEQ ID NO: 420)
EGL-13: MSRRRKANPTKLSENAKKLAKEVEN

Vectors

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA.

The term “expression cassette” as used herein refers to genetic sequences within a vector which can express a RNA, and subsequently a protein. The nucleic acid cassette contains the gene of interest, e.g., a modified looped protein. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit. In some embodiments, the nucleic acid cassette contains the sequence of a modified looped protein.

Examples of vectors include, without limitation, plasmids, phagemids, cosmids, transposons, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, the coding sequences of the modified looped proteins disclosed herein can be ligated into such expression vectors for the expression of the modified looped protein in host cells. In some embodiments, non-viral vectors are used to deliver one or more polynucleotides contemplated herein to a host cell.

In embodiments, the vector is a non-integrating vector, including but not limited to, an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus.

Typically, the host cell comprises the viral replication transactivator protein that activates the replication.

In embodiments, a polynucleotide is introduced into a target or host cell using a transposon vector system. In certain embodiments, the transposon vector system comprises a vector comprising transposable elements and a polynucleotide contemplated herein; and a transposase. In one embodiment, the transposon vector system is a single transposase vector system, see, e.g., WO 2008/027384. Exemplary transposases include, but are not limited to: piggyBac, Sleeping Beauty, Mos1, Tc1/mariner, Tol2, mini-Tol2, Tc3, MuA, Himar I, Frog Prince, and derivatives thereof.

The piggyBac transposon and transposase are described, for example, in U.S. Pat. No. 6,962,810, which is incorporated herein by reference in its entirety. The Sleeping Beauty transposon and transposase are described, for example, in Izsvak et al., J. Mol. Biol. 302: 93-102 (2000), which is incorporated herein by reference in its entirety. The Tol2 transposon which was first isolated from the medaka fish Oryzias latipes and belongs to the hAT family of transposons is described in Kawakami et al. (2000). Mini-Tol2 is a variant of Tol2 and is described in Balciunas et al. (2006). The Tol2 and Mini-Tol2 transposons facilitate integration of a transgene into the genome of an organism when co-acting with the Tol2 transposase. The Frog Prince transposon and transposase are described, for example, in Miskey et al., Nucleic Acids Res. 31:6873-6881 (2003).

The “control elements” or “regulatory sequences” present in an expression vector are those non-translated regions of the vector (e.g., origin of replication, selection cassettes, promoters, enhancers, translation initiation signals (Shine Dalgarno sequence or Kozak sequence) introns, a polyadenylation sequence, 5′ and 3′ untranslated regions) which interact with host cellular proteins to carry out transcription and translation. Such elements may vary in their strength and specificity. Depending on the vector system and host utilized, any number of suitable transcription and translation elements, including ubiquitous promoters and inducible promoters may be used. In some embodiments, the polynucleotide of interest is operably connected to a control element or regulatory sequence. “Operably connected” refers to a juxtaposition wherein the components so described are in a relationship permitting them to function in their intended manner. For instance, a promoter is operably linked to a polynucleotide sequence if the promoter affects the transcription or expression of the polynucleotide sequence.

In some embodiments, the polynucleotide of interest is operably linked to a promoter sequence. The term “promoter” as used herein refers to a recognition site of a polynucleotide (DNA or RNA) to which an RNA polymerase binds. An RNA polymerase initiates and transcribes polynucleotides operably linked to the promoter. Illustrative ubiquitous promoters suitable for use in particular embodiments include, but are not limited to, a cytomegalovirus (CMV) immediate early promoter, a viral simian virus 40 (SV40) (e.g., early or late) promoter, a spleen focus forming virus (SFFV) promoter, a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1α) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4A1 (EIF4A1) promoter, a heat shock 70 kDa protein 5 (HSPA5) promoter, a heat shock protein 90 kDa beta, member 1 (HSP90B1) promoter, a heat shock protein 70 kDa (HSP70) promoter, a β-kinesin (β-KIN) promoter, the human ROSA 26 locus (Irions et al., Nature Biotechnology 25, 1477-1482 (2007)), a Ubiquitin C (UBC) promoter, a phosphoglycerate kinase-1 (PGK) promoter, a cytomegalovirus enhancer/chicken j-actin (CAG) promoter, a j-actin promoter and a myeloproliferative sarcoma virus enhancer, negative control region deleted, dl587rev primer-binding site substituted (MND) promoter (Challita et al., J Virol. 69(2):748-55 (1995)).

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to, those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

### Protein Expression Systems

In some embodiments, a vector comprising an expression cassette comprising a nucleic acid sequence encoding a modified looped protein described herein is introduced into a host cell that is capable of expressing the encoded modified looped protein. Exemplary host cells include Chinese Hamster Ovary (CHO) cells, HEK 293 cells, BHK cells, murine NSO cells, or murine SP2/0 cells, and E. coli cells. The expressed protein is then purified from the culture system using any one of a variety of methods known in the art (e.g., Protein A columns, affinity chromatography, size-exclusion chromatography, and the like).

Numerous expression systems exist that are suitable for use in producing the modified loop proteins described herein. Eukaryote-based systems in particular can be employed to produce nucleic acid sequences, or their cognate polypeptides, proteins and peptides. Many such systems are commercially and widely available.

In some embodiments, the modified loop proteins described herein are produced using Chinese Hamster Ovary (CHO) cells following standardized protocols. Alternatively, for example, transgenic animals may be utilized to produce the modified loop proteins described herein, generally by expression into the milk of the animal using well established transgenic animal techniques. Lonberg N. Human antibodies from transgenic animals. Nat Biotechnol. 2005 September; 23(9): 1117-25; Kipriyanov et al. Generation and production of engineered antibodies. Mol Biotechnol. 2004 January; 26(1):39-60; See also Ko et al., Plant biopharming of monoclonal antibodies. Virus Res. 2005 July; 111(1):93-100.

The insect cell/baculovirus system can produce a high level of protein expression of a heterologous nucleic acid segment, such as described in U.S. Pat. Nos. 5,871,986 and 4,879,236, both incorporated herein by reference in their entireties, and which can be bought, for example, under the name MAXBAC® 2.0 from Invitrogen and BACPACK™ Baculovirus expression system from Clonotech.

Other examples of expression systems include Stratagene's Complete Control Inducible Mammalian Expression System, which utilizes a synthetic ecdysone-inducible receptor. Another example of an inducible expression system is available from Invitrogen, which carries the T-REX™ (tetracyclineregulated expression) System, an inducible mammalian expression system that uses the full-length CMV promoter. Invitrogen also provides a yeast expression system called the Pichia methanolica Expression System, which is designed for high-level production of recombinant proteins in the methylotrophic yeast Pichia methanolica. One of skill in the art would know how to express vectors such as an expression construct comprising a nucleic acid sequence encoding a modified looped protein described herein, to produce its encoded nucleic acid sequence or its cognate polypeptide, protein, or peptide. See, generally, Recombinant Gene Expression Protocols By Rocky S. Tuan, Humana Press (1997), ISBN 0896033333; Advanced Technologies for Biopharmaceutical Processing By Roshni L. Dutton, Jeno M. Scharer, Blackwell Publishing (2007), ISBN 0813 805171; Recombinant Protein Production With Prokaryotic and Eukaryotic Cells By Otto-Wilhelm Merten, Contributor European Federation of Biotechnology, Section on Microbial Physiology Staff, Springer (2001), ISBN 0792371372.

As an alternative, proteins of the present invention can be synthesized by exclusive solid phase synthesis, partial solid phase methods, fragment condensation or classical solution synthesis. These synthesis methods are well-known to those of skill in the art (see, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept. Prot. 3:3 (1986), Atherton et al., Solid Phase Peptide Synthesis: A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, for example, Dawson et al., Science 266:776 (1994), Hackeng et al., Proc. Nat'l Acad. Sci. USA 94:7845 (1997), Dawson, Methods Enzymol. 287: 34 (1997), Muir et al, Proc. Nat'l Acad. Sci. USA 95:6705 (1998), and Severinov and Muir, J. Biol. Chem. 273:16205 (1998)). In one example of expressed protein ligation, a recombinantly expressed protein is cleaved from an intein and the protein is ligated to a peptide containing an N-terminal cysteine having an unoxidized sulfhydryl side chain, by contacting the protein with the peptide in a reaction solution containing a conjugated thiophenol. This forms a C-terminal thioester of the recombinant protein which spontaneously rearranges intramolecularly to form an amide bond linking the protein to the peptide. See, generally, Muir, T W et al Expressed Protein Ligation: A General Method for Protein Engineering, PNAS (1998) 95(12)6705-6710; U.S. Pat. No. 6,849,428; US Pub. 2002/0151006; Bondalapati, et al., Expanding the chemical toolbox for the synthesis of large and uniquely modified proteins. (2016) Nature Chemistry volume 8, pages 407-418; Amy E. Rabideau and Bradley Lether Pentelute*. Delivery of Non-Native Cargo into Mammalian Cells Using Anthrax Lethal Toxin. ACS Chem. (2016) Biol., 11(6) 1490-1501; and Weidmann et al., Copying Life: Synthesis of an Enzymatically Active Mirror-Image DNA-Ligase Made of D-Amino Acids. Cell Chemical Biology, (2019 May 16) 26(5); 616-619.

### Compositions and Methods of Administration and Treatment

In some embodiments, this disclosure describes compositions that include a degradation construct and or degradation compound described herein. including at least one of the antibodies described herein. In embodiments, the composition is a pharmaceutical composition.

The degradation constructs, degradation compounds, and pharmaceutical compositions thereof described herein can be administered to a subject to treat or prevent a disease or condition, or one or more symptoms of a disease or condition. In embodiments, a therapeutically-effective amount of a degradation compound, or pharmaceutical composition described herein is administered to a subject to treat and/or prevent a disease or condition or progression thereof.

The amount, duration and frequency of administration of a pharmaceutical composition or construct described herein can depend on several factors including, but not limited to, the health of the subject, the disease or condition being treated, the grade or level of a specific disease or condition of the patient, whether the subject has been administered any additional therapeutics, and the like.

The degradation compounds and degradation constructs disclosed herein, and compositions containing them, may be administered by any suitable method and technique presently or prospectively known to those skilled in the art. For example, the disclosed compounds can be formulated in a physiologically- or pharmaceutically-acceptable form and administered by any suitable route known in the art including, for example, oral and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The degradation compounds disclosed herein, and compositions comprising them, can also be administered utilizing liposome technology, slow release capsules, implantable pumps, and biodegradable containers. These delivery methods can, advantageously, provide a uniform dosage over an extended period of time. The degradation compounds can also be administered in their salt derivative forms.

The degradation compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (2020) describes formulations that can be used in connection with the disclosed methods. In general, the degradation compounds disclosed herein can be formulated such that an effective amount of the compound is combined with a suitable carrier in order to facilitate effective administration of the degradation compound. The compositions used can also be in a variety of forms. These include, for example, solid, semi-solid, and liquid dosage forms, such as tablets, pills, powders, liquid solutions or suspension, injectable and infusible solutions, and sprays. The preferred form depends on the intended mode of administration and therapeutic application. In particular embodiments, the constructs are formulated as a liquid dosage form, such as an injectable and infusible solutions.

In some embodiments, the compositions also include pharmaceutically-acceptable carriers and diluents. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject constructs based on the weight of the total composition including carrier or diluent.

Formulations suitable for parenteral administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Degradation compounds and compositions disclosed herein can be administered intravenously, intramuscularly, or intraperitoneally by infusion or injection. Solutions of the degradation compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions may be prepared by incorporating a construct disclosed herein in the required amount in the appropriate solvent with various other ingredients enumerated above, as required, followed by filter sterilization. In the case of sterile powders for the preparation of sterile injectable solutions, methods of preparation including vacuum drying and freeze drying techniques, which yield a powder of the active ingredient plus any additional desired ingredient present in the previously sterile-filtered solutions.

Useful dosages of the constructs and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex, and extent of the disease in the patient and can be determined by one of skill in the art. The dosage can be adjusted by the individual physician in the event of any counter indications. Dosage can vary, and can be administered in one or more dose administrations daily, for one or several days.

Also disclosed are kits that comprise a degradation compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a degradation compound disclosed herein is provided in the kit as a liquid or solution.

In one embodiment, the kit comprises an ampoule or syringe containing a degradation compound disclosed herein in liquid or solution form.

The degradation compounds disclosed herein may be used to treat a wide range of diseases. In embodiments, the degradation compounds disclosed herein can be used in the treatment of a wide range of diseases where protein knockdown is indicated. Such diseases include proteostatic diseases and diseases arising from undesired intracellular protein activity (for example, in the treatment of cancer, where the target protein nay be an oncoprotein). A “proteostatic disease” is refers to a set of diseases mediated, at least in part, by deficiencies in proteostasis. The term therefore covers aggregative and misfolding proteostatic diseases, including in particular neurodegenerative disorders (e.g., Parkinson's disease, Alzheimer's disease and Huntington's disease).

In some embodiments, the is disease selected from connective tissue disorders (for example, achondrogenesis type II, lysyl-hydroxylase deficiency), dwarfism (for example, kniest dysplasia, achondroplasia, thanatophoric dysplasia, spondyloepimetaphyseal dysplasia (Strudwick type), spondyloepimetaphyseal dysplasia (congenital type), Nance-Insley syndrome), congenital disorders (for example, Gunther disease (congenital erythropoietic porphyria (CEP)), Noonan syndrome, apert syndrome, congenital heart disease, Cayler cardiofacial syndrome (BAVD) (asymmetric crying facies (ACF)), Weissenbacher-Zweymuller syndrome (WZS), congenital hyperthyroidism, conotruncal anomaly face syndrome (CTAF), osteogenesis imperfecta (brittle bone disease), congenital absence of the vas deferens (CAVD), Treacher Collins syndrome (TCS), acrocephaly (oxycephaly), broad thumb-hallux syndrome (Rubinstein-Taybi Syndrome), congenital methemoglobinemia, Benjamin syndrome), genetic or chromosomal disorders (for example, Gaucher disease type 2, aceruloplasminemia, angiokeratoma corporis diffusum, incontinentia pigmenti (Bloch-Sulzberger disease), hypochondroplasia, familial dysautonomia (FD) (Riley-Day syndrome), Wolf-Hirschhorn syndrome (Pitt syndrome), Smith-Lemli-Opitz syndrome, Alström syndrome, Stickler syndrome, fragile X syndrome, Osler-Weber-Rendu syndrome (hereditary hemorrhagic telangiectasia), hereditary spastic paraplegias, muscular dystrophy (Duchenne and Becker types), autosomal recessive familial amyotrophic lateral sclerosis (RFALS), adrenogenital syndrome, adrenoleukodystrophy (ALD), Sandhoff Disease, campomelic dysplasia (CMD), Lesch-Nyhan syndrome, Hutchinson-Gilford progeria syndrome (progeria), infantile-onset ascending hereditary spastic paralysis, severe achondroplasia with developmental delay and acanthosis nigricans (SADDAN), xeroderma pigmentosum, Joubert syndrome, Cowden syndrome (Multiple hamartoma syndrome), Birt-Hogg-Dubé syndrome (BHD), Bloom syndrome, Wilson's disease, Coffin-Lowry syndrome, Jackson-Weiss syndrome, alpha-1-antitrypsin deficiency, androgen insensitivity syndrome (AIS), Factor V Leiden Thrombophilia, micro syndrome (Warburg-Sjo-Fledelius syndrome), McCune-Albright syndrome, polycystic kidney disease, Pseudoxanthoma elasticum (Gronblad-Strandberg syndrome), cystic fibrosis, Otospondylomegaepiphyseal dysplasia (OSMED), Beare-Stevenson cutis gyrate syndrome, X-linked severe combined immunodeficiency (X-SCID), Bonnevie-Ullrich syndrome (Turners syndrome), Anderson-Fabry disease, Cri du Chat Syndrome (Lejeune's Syndrome), Down's Syndrome (Trisomy 21), Klinefelter syndrome, Von Hippel-Lindau disease, Cockayne syndrome (CS) (Neill-Dingwall Syndrome), Triple X syndrome (Trisomy X), Roussy-Levy syndrome, Peutz-Jeghers syndrome, chondrodystrophy syndrome, Familial Mediterranean Fever (FMF), Insley-Astley syndrome, Muenke syndrome (FGFR3-related craniosynostosis), Lynch syndrome (hereditary nonpolyposis colorectal cancer (HNPCC)), Marfan syndrome, Neurofibromatosis (NF) (types 1, 2 and Schwannomatosis), Hemochromatosis (Bronze Diabetes/Bronzed Cirrhosis), Waardenburg syndrome, Noack syndrome (Pfeiffer syndrome), Mowat-Wilson syndrome, Ehlers-Danlos syndrome, Usher syndrome (Hallgren syndrome), Li-Fraumeni syndrome (SBLA), Prader-Willi syndrome, Crouzon Syndrome (craniofacial dysarthtosis), Bourneville Disease (tuberous sclerosis), fibrocystic disease of the pancreas, Shprintzen-Goldberg syndrome, familial adenomatous polyposis (FAP), chronic granulomatous disease (CGD), Hyperandrogenism, Menkes Disease (MNK), Adenomatous Polyposis coli (APC)), metabolic disorders (for example, galactosemia, adenylosuccinate lyase deficiency, sphingolipidosis, porphyria cutanea tarda (PCT), Niemann-Pick disease, alkaptonuria, alkaptonuroc ochronosis, variegate porphyria (South African genetic porphyria), erythropoietic protoporphyria, erythropoietic porphyria, tetrahydrobiopterin deficiency (THBD) (BH4D), acute intermittent porphyria, aminolevulinic acid dehydratase deficiency porphyria (ALA dehydratase deficiency), ALAD Deficiency Porphyria (ADP), hyperoxaluria, primary hyperoxaluria, propionic academia, Burger-Grutz syndrome, Tay-Sachs disease, hepatoerythropoietic porphyria (HIIEP)), neurological, neurodegenerative diseases and central nervous system disorders (for example, Parkinson's disease, Alzheimer's disease, myotonic dystrophy (type 1 and type 2), dyskinesias, prion disease, schizophrenia, bipolar disorder. Krabbe disease (globoid cell leukodystrophy), Dejerome-Sottas disease, lacunar dementia, vascular dementia, Machado-Joseph disease (MJD), Huntington's disease, Alexander disease, progressive chorea, muscular atrophy, progressive muscular atrophy (PMA), bulbospinal muscular atrophy (Kennedy's disease), Friedreich's ataxia, spinal muscular atrophy, distal spinal muscular atrophy, canavan disease, primary senile degenerative dementia, hereditary neuropathy with liability to pressure palsy (HNPP) (tomaculous neuropathy), Creutzfeldt-Jakob disease, spinocerebellar ataxia, ataxia telangiectasia (Louis-Bar syndrome), amyotrophic lateral sclerosis (ALS), stroke, Rett syndrome, De Grouchy syndrome, motor neuron disease, AD-ID, autism and major depressive disorder (MDD)), diseases of the lung (for example, emphysema), dental diseases (for example, amelogenesis imperfecta), diseases or disorders of the blood (for example, haemochromatosis, anemia, beta thalassemia (Cooley's anemia), haemophilia, sickle-cell anaemia, hypochromic anemia, hereditary coproporphyria (HCP), X-linked sideroblastic anemia, methemoglobinemia, primary pulmonary hypertension (PPH), pulmonary arterial hypertension (PAH), ischemia, acute and chronic CNS injury ischemia), eye diseases (for example, angiomatosis retinae, retinoblastoma (R^{b}), retinal degeneration), heart diseases (for example, cardiomyopathy, myocardial infarction), liver diseases (for example, neonatal hemochromatosis), inflammatory disorders (for example, hepatitis, inflammatory bowel disease (IBD), ulcerative colitis and gastritis), autoimmune diseases (for example, multiple sclerosis (MS), and type 1 diabetes), kidney diseases (for example, autosomal dominant polycystic kidney disease (ADPKD)), lung diseases, movement disorders, skin pigmentation disorders, speech and communication disorders, and thyroid diseases.

In some embodiments, the degradation compounds disclosed herein may be used for the treatment of conditions or diseases selected from inflammation, hepatitis, ulcerative colitis, gastritis, autoimmunity, inflammation, restenosis, stroke, heart failure, neurodegenerative conditions such as Alzheimer's disease, Parkinson's disease, Huntington's disease, myotonic dystrophy, and amyotrophic lateral sclerosis, AIDS, ischemia such as traumatic brain injury, spinal cord injury, cerebral ischemia, cerebral ischemia/reperfusion (I/R) injury, acute and chronic CNS injury ischemia, stroke or myocardial infarction, degenerative diseases of the musculoskeletal system such as osteoporosis, autoimmune diseases such as multiple sclerosis (MS) and Type I diabetes, and eye diseases such as retinal degeneration which result from loss of control of programmed cell death.

In some embodiments, the degradation compounds may be used to treat various cancers. The cancers may be selected from primary tumors (e.g., cancer cells at the originating site), local invasion (cancer cells which penetrate and infiltrate surrounding normal tissues in the local area), and metastatic (or secondary) tumors—e.g., tumors that have formed from malignant cells which have circulated through the bloodstream (haematogenous spread) or via lymphatics or across body cavities (trans-coelomic) to other sites and tissues in the body. Examples of cancers (and their benign counterparts) which may be treated (or inhibited) include, but are not limited to tumors of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, bowel, colon, rectum and anus), liver (hepatocellular carcinoma), gall bladder and biliary system, exocrine pancreas, kidney (renal cell carcinoma), lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia (ALL), chronic lymphocytic leukemia (CLL), chronic myeloid leukaemia (CML), B-cell lymphomas such as diffuse large B-cell lymphoma (DLBCL), Pre-B acute lymphoblastic leukaemia, Pre-B lymphomas, Large B-cell lymphomas, B-Cell acute lymphoblastic leukaemia, Philadelphia chromosome positive acute lymphoblastic leukaemia, Philadelphia chromosome positive chronic myeloid leukaemia, follicular lymphoma, Burkitt's lymphoma, mantle cell lymphoma, T-cell lymphomas and leukaemias, T-lineage acute lymphoblastic leukaemia (T-ALL), T-lineage lymphoblastic lymphoma (T-LL), peripheral T-cell lymphoma, adult T-cell leukaemia, natural killer (NK) cell lymphomas, Hodgkin's lymphomas, Non-Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenous leukemia (AML), chronic myelogenous leukemia (CML), chronic myelomonocytic leukemia (CMML), hypereosinophilic syndrome, myeloproliferative disorders such as polycythaemia vera, essential thrombocythaemia and primary myelofibrosis, myeloproliferative syndrome, myelodysplastic syndrome, and promyelocytic leukemia): tumors of mesenchymal origin (for example sarcomas of soft tissue, bone or cartilage such as osteosarcomas, fibrosarcomas, chondrosarcomas, rhabdomyosarcomas, leiomyosarcomas, liposarcomas, angiosarcomas, myosarcoma, Kaposi's sarcoma, Ewing's sarcoma, synovial sarcomas, epithelioid sarcomas, gastrointestinal stromal tumors, benign and malignant histiocytomas, and dermatofibrosarcoma protuberans; neural crest cell-derived tumors including melanocytic tumors (for example, malignant melanoma or uveal melanoma), tumors of peripheral and cranial nerves, peripheral neuroblastic tumors (for example, neuroblastoma), embryonal tumors of the CNS, paraganglioma; tumors of the central or peripheral nervous system (for example, astrocytomas, gliomas and glioblastomas, gangliogliomas, ganglioneuromas, oligodendroglioma, meningiomas, ependymomas, pineal tumors and schwannomas); endocrine tumors (for example, pituitary tumors, adrenal tumors, islet cell tumors, parathyroid tumors, carcinoid tumors and medullary carcinoma of the thyroid); ocular and adnexal tumors (for example, retinoblastoma); germ cell and trophoblastic tumors (for example, teratomas, seminomas, dysgerminomas, hydatidiform moles and choriocarcinomas); and pediatric and embryonal tumors (for example medulloblastoma, neuroblastoma, Wilms tumor, and primitive neuroectodermal tumors); or syndromes, congenital or otherwise, which leave the patient susceptible to malignancy (for example, Xerodemia Pigmentosum).

Specific types of cancers or malignant tumors, either primary or secondary, that can be treated using the disclosed degradation compounds include breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglioneuromas, hyperplastic corneal nerve tumor, marfanoid habitus tumor, Wilm's tumor, seminoma, ovarian tumor, leiomyomater, cervical dysplasia and in situ carcinoma, neuroblastoma, retinoblastoma, soft tissue sarcoma, malignant carcinoid, topical skin lesion, mycosis fimgoide, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, epidermoid carcinomas, and other carcinomas and sarcomas.

In embodiments, the degradation compounds disclosed herein can be used to treat infectious diseases, for example, diseases associated with bacterial, viral, fungal and/or parasitic infection. In embodiments, the degradation compounds constructs can be used in the treatment of infectious diseases. In embodiments, the degradation compounds can be used in the treatment of infectious diseases. In embodiments, the target protein comprises a pathogenic microorganism, including, but not limited to, bacteria, viruses, fungi, or intracellular or extracellular parasites. In embodiments, the target protein comprises a surface target protein, for example, a protein or carbohydrate or mixture thereof, of an infectious agent.

In embodiments, a method is provided for treating a disease or disorder associated with Interferon Regulatory Factor-5 (IRF-5). The method includes administering a compound or composition described herein to a subject in need thereof. In embodiments, the method comprises administering a therapeutically effective amount of a compound or composition described herein to a subject in need thereof.

In embodiments, the disease or disorder is associated with IRF-5 genetic variation. In embodiments, the disease or disorder is associated with a genetic mutation in the IRF-5 gene. In embodiments, the genetic mutation in IRF-5 results IRF-5 overexpression. In embodiments, the genetic mutation results in alternate isoform expression. In embodiments, the disease or disorder is associated with IRF-5 overexpression. In embodiments, the disease or disorder is associated with IRF-5 isoform expression.

In embodiments, a method is provided for treating inflammation, autoantibody production, inflammatory cell infiltration, collagen deposits, or inflammatory cytokine production in a patient.

In embodiments, a method of downregulating IRF-5 expression in a patient is provided. In embodiments, IRF-5 expression in a macrophage is reduced. In embodiments, IRF-5 expression in a Kupffer cell is reduced. In embodiments, IRF-5 expression in the gastrointestinal tract is reduced. In embodiments, expression of IRF-5 in the liver is reduced. In embodiments, expression of IRF-5 in the lungs is reduced. In embodiments, expression of IRF-5 in the kidneys is reduced. In embodiments, expression of IRF-5 in the joints is reduced. In embodiments, expression of IRF-5 in the central nervous system is reduced.

In embodiments, the compounds disclosed herein are used for treating a disease associated with IRF-5. Examples of diseases associated with IRF-5 include, but are not limited to, inflammatory bowel disease (IBD), ulcerative colitis, Crohn's disease, systemic lupus erythematosus (SLE), rheumatoid arthritis, primary biliary cirrhosis, systemic sclerosis, Sjogren's syndrome, multiple sclerosis, scleroderma, interstitial lung disease (SSc-ILD), polycystic kidney disease (PKD), chronic kidney disease (CKD), Nonalcoholic steatohepatitis (NASH), liver fibrosis, asthma, and severe asthma. In embodiments, the compounds disclosed herein are used to reduce inflammation, cirrhosis, fibrosis, proteinuria, joint inflammation, autoantibody production, inflammatory cell infiltration, collagen deposits, or inflammatory cytokine production in a patient. In embodiments, the compounds disclosed herein are used to reduce inflammation in the gastrointestinal tract, diarrhea, pain, fatigue, abdominal cramping, blood in the stool, intestinal inflammation, disruption of the epithelial barrier of the gastrointestinal tract, dysbiosis, increased bowel frequency, tenesmus or painful spasms of the anal sphincter, constipation, or unintended weight loss.

In embodiments, the compounds disclosed herein are used for treating an inflammatory disease. “Inflammatory disease” refers to diseases in which activation of the innate or adaptive immune response is a prominent contributor to the clinical condition. Inflammatory diseases include, but are not limited to, acne vulgaris, asthma, COPD, autoimmune diseases, celiac disease, chronic (plaque) prostatitis, glomerulonephritis, hypersensitivities, inflammatory bowel diseases (IBD, Crohn's disease, ulcerative colitis), pelvic inflammatory disease, reperfusion injury, rheumatoid arthritis, sarcoidosis, transplant rejection, vasculitis, interstitial cystitis, atherosclerosis, allergies (type 1, 2, and 3 hypersensitivity, hay fever), inflammatory myopathies, as systemic sclerosis, and include dermatomyositis, polymyositis, inclusion body myositis, Chediak-Higashi syndrome, chronic granulomatous disease, Vitamin A deficiency, cancer (solid tumor, gallbladder carcinoma), periodontitis, granulomatous inflammation (tuberculosis, leprosy, sarcoidosis, and syphilis), fibrinous inflammation, purulent inflammation, serous inflammation, ulcerative inflammation, and ischemic heart disease, type I diabetes, and diabetic nephropathy.

In embodiments, the compounds disclosed herein are used for treating an autoimmune disease. “Autoimmune disease” refers to a disease or disorder in which a patient's immune system attacks the patient's own tissues. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); systemic sclerosis (scleroderma); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, dermatomyositis; granulomatosis and vasculitis; primary biliary cirrhosis; pernicious anemia (Addison's disease); autoimmune gastritis; autoimmune hepatitis; diseases involving leukocyte diapedesis; central nervous system (CNS) inflammatory disorder; vitiligo; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

In embodiments, the compounds disclosed herein are used for treating an autoimmune disease such as systemic lupus erythematosus (SLE), systemic sclerosis (scleroderma), polymyositis/dermatomyositis, Crohn's disease, ulcerative colitis, rheumatoid arthritis, Sjogren's syndrome, autoimmune encephalomyelitis, nonalcoholic steatohepatitis (NASH), sarcoidosis, Behcet's disease, myasthenia gravis, lupus nephritis, inflammatory bowel disease (IBD), ankylosing spondylitis, primary biliary cirrhosis, colitis, pulmonary fibrosis, antiphospholipid syndrome, or psoriasis.

In embodiments, the compounds disclosed herein are used for treating cardiovascular disease. In embodiments, the cardiovascular disease is associated with inflammation. In embodiments, the cardiovascular disease includes systemic scleroderma. In embodiments, the cardiovascular disease includes aneurysm; angina; atherosclerosis; cerebrovascular accident (Stroke); cerebrovascular disease; congestive heart failure; coronary artery disease; myocardial infarction (heart attack); or peripheral vascular disease. In embodiments, the cardiovascular disease includes atherosclerosis.

In embodiments, the compounds disclosed herein are used for treating a gastrointestinal disease. In embodiments, the gastrointestinal disease includes Crohn's disease, primary biliary cirrhosis, sclerosing cholangitis, ulcerative colitis, inflammatory bowel disease, or Sjogren's syndrome.

In embodiments, the compounds disclosed herein are used for treating a urinary system disease. In embodiments, the urinary system disease includes systemic lupus erythematosus or systemic scleroderma.

In embodiments, the compounds disclosed herein are used for treating a genetic, familial, or congenital disease. In embodiments, the genetic, familial or congenital disease includes Crohn's disease, primary biliary cirrhosis, systemic scleroderma, systemic lupus erythematosus, ulcerative colitis, psoriasis, or inflammatory bowel disease.

In embodiments, the compounds disclosed herein are used for treating an endocrine system disease. In embodiments, the endocrine system disease includes thyroid gland adenocarcinoma, primary biliary cirrhosis, sclerosing cholangitis, or hypothyroidism.

In embodiments, the compounds disclosed herein are used for treating a cell proliferation disorder. In embodiments, the cell proliferation disorder includes primary biliary cirrhosis, thyroid gland adenocarcinoma, or neoplasm.

In embodiments, the compounds disclosed herein are used for treating an immune system disease. In embodiments, the immune system disease includes Sjögren's syndrome, inflammatory bowel disease, psoriasis, myositis, systemic scleroderma, autoimmune disease, systemic lupus erythematosus, rheumatoid arthritis, Crohn's disease, ulcerative colitis, or ankylosing spondylitis.

In embodiments, the compounds disclosed herein are used for treating a hematologic disease. In embodiments, the hematologic disease includes systemic lupus erythematosus.

In embodiments, the compounds disclosed herein are used for treating a musculoskeletal or connective tissue disease. In embodiments, the musculoskeletal or connective tissue disease includes myositis, systemic scleroderma, systemic lupus erythematosus, rheumatoid arthritis, ankylosing spondylitis or adolescent idiopathic scoliosis.

In embodiments, the compounds disclosed herein are used for treating neuroinflammatory disease. In embodiments, the neuroinflammatory disease or disorder includes inflammation due to traumatic brain injury, acute disseminated encephalomyelitis (ADEM), autoimmune encephalitis, acute optic neuritis (AON), chronic meningitis, anti-myelin oligodendrocyte glycoprotein (MOG) disease, transverse myelitis, neuromyelitis optica (NMO), Alzheimer's disease, Parkinson's disease or multiple sclerosis (MS).

In embodiments, the compounds disclosed herein are used for treating inflammation due to infection by microorganisms such as viruses, bacteria, fungi or parasites.

In embodiments, the compounds disclosed herein are used for treating a disease associated with fibrosis, which is referred to herein as a fibrotic disease. “Fibrosis” refers to a pathological formation of fibrous connective tissue, for example, due to injury, irritation, or chronic inflammation and includes fibroblast accumulation and collagen deposition in excess of normal amounts in a tissue. “Fibrotic disease” refers to a disease associated with pathological fibrosis. Examples of fibrotic disease include, but are not limited to, idiopathic pulmonary fibrosis; scleroderma; scleroderma of the skin; scleroderma of the lungs; a collagen vascular disease (e.g., lupus; rheumatoid arthritis; scleroderma); genetic pulmonary fibrosis (e.g., Hermansky-Pudlak Syndrome); radiation pneumonitis; asthma; asthma with airway remodeling; chemotherapy-induced pulmonary fibrosis (e.g., bleomycin, methotrextate, or cyclophosphamide-induced); radiation fibrosis; Gaucher's disease; interstitial lung disease; retroperitoneal fibrosis; myelofibrosis; interstitial or pulmonary vascular disease; fibrosis or interstitial lung disease associated with drug exposure; interstitial lung disease associated with exposures such as asbestosis, silicosis, and grain exposure; chronic hypersensitivity pneumonitis; an adhedsion; an intestinal or abdominal adhesion; cardiac fibrosis; kidney fibrosis; cirrhosis; and nonalcoholic steatohepatitis (NASH)-induced fibrosis. In embodiments, the fibrotic disease includes nonalcoholic steatohepatitis NASH.

In embodiments, the compounds disclosed herein are used for treating a respiratory or thoracic disease such as systemic scleroderma. In embodiments, the compounds disclosed herein are used for treating an integumentary system disease such as psoriasis or systemic scleroderma. In embodiments, the compounds disclosed herein are used for treating a disease of the visual system such as Sjögren's syndrome or systemic scleroderma. In embodiments, the compounds disclosed herein are used for treating a disease associated with eosinophil count, glomular filtration rate, systolic blood pressure, eosinophil percentage of leukocytes. In embodiments, the compounds disclosed herein are used for treating an ulcer disease or an oral ulcer.

Non-limiting examples of target intracellular proteins that may be targeted by degradation compounds disclosed herein include eukaryotic, prokaryotic, fungal and viral proteins. The target proteins therefore include, but are not limited to, transport (nuclear, carrier, ion, channel, electron, protein), behavioral, receptor, cell death, cell differentiation, cell surface, structural proteins, cell adhesion, ceil communication, cell motility, enzymes, cellular function (helicase, biosynthesis, motor, antioxidant, catalytic, metabolic, proteolytic), membrane fusion, development, proteins regulating biological processes, proteins with signal transducer activity, receptor activity, isomerase activity, enzyme regulator activity, chaperone regulator, binding activity, transcription regulator activity, translation regulator activity, structural molecule activity, ligase acuity, extracellular organization activity, kinase activity, biogenesis activity, ligase activity, and nucleic acid binding activity.

In some embodiments, target proteins may be selected from, and are therefore not limited to, DNA methyl transferases, AKT pathway proteins, MAPK/ERK pathway proteins, tyrosine kinases, epithelial growth factor receptors (EGFRs), fibroblast growth factor receptors (FGFRs), vascular endothelial growth factor receptors (VEGFRs), erythropoietin-producing human hepatocellular receptors (Ephs), tropomyosin receptor kinases, tumor necrosis factors, apoptosis regulator Bcl-2 family proteins, Aurora kinases, chromatin, G-protein coupled receptors (GPCRs), NF-κB pathway, HCV proteins, HIV proteins, Aspartyl proteases, Histone deacetylases (HDACs), glycosidases, lipases, histone acetyltransferase (HAT), cytokines and hormones.

Specific target proteins may be selected from IRF-5, β-catenin, ERK1/2, ERK5, A-Raf, B-Raf, C-Raf, c-Mos, Tpl2/Cot, MEK, MKK1, MKK2, MKK3, MKK4, MKK5, MKK6, MKK7, TYK2, JNK1, JNK2, JNK3, MEKK1, MEKK2, MEKK3, MEKK4, ASK1, ASK2, MLK1, MLK2, MLK3, p38 α, p38 β, p38 γ, p38 δ, BRD2, BRD3, BRD4, phosphatidyl inositol-3 kinase (PI3K), AKT, Protein kinase A (PKA), Protein Kinase B (PKB), Protein kinase C (PKC), mTOR, PDK-1, p70 S6 kinase, forkhead translocation factor, MELK, eIF4E, Hsp90, Hsp70, Hsp60, topoisomerase type I, topoisomerase type II, DNMT1, DNMT3A, DNMT3B, Cdk11, Cdk2, Cdk3, Cdk4, Cdk5, Cdk6, Cdk7, α-tubulin, β-tubulin, γ-tubulin, δ-tubulin, ε-Tubulin, Janus Kinases (JAK1, JAK2, JAK3), ABL1, ABL2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, HER2/neu, Her3, Her4, ALK, FGFR1, FGFR2, FGFR3, FGFR4, IGF1R, INSR, INSRR, VEGFR-1, VEGFR-2, VEGFR-3, FLT-3, FLT4, PDGFRA, PDGFRB, CSF1R, Ax1, IRAK4, SCFR, Fyn, MuSK, Btk, CSK, PLK4, Fes, MER, c-MET, LMTK2, FRK, ILK, Lek, TIE1, FAK, P1K6, TNN13, ROSCCK4, ZAP-70, c-Src, Tec, Lyn, TrkA, TrkB, TrkC, RET, ROR1, ROR2, ACK1, Syk, MDM2, MDM4, HRas, KRas, NRas, ROCK, PI3K, BACE1, BACE2, CTSD, CTSE, NAPSA, PGC, Renin, MMSET, Aurora A kinase, Aurora B kinase, Aurora C kinase, farnesyltransferase, telomerase, adenylyly cyclase, cAMP phosphodiesterase, PARP1, PARP2, PARP4, PARP-5a, PARP-5b, PKM2, Keap1, Nrf2, TNF, TRAIL, OX40L Lymphotoxin-alpha, IFNAR1, IFNAR2, IFN-α, IFN-β, IFN-γ, IFNLR1, CCL3, CCL4, CCL5, IL1α, IL1β, IL-2, IL-2, IL-4, IL-5, IL-6, IL-7, IL-9, IL-9, IL-10, IL-11, IL-12, IL-13, IL-15, IL-17, Bcl-2, Bcl-xL, Bax, HCV helicase, E1, E2, p7, NS2, NS3, NS4A, NS4B, NS5A, NS5B, NF-κB1, NF-κB2, RelA, RelB, c-Rel, RIP1, ACE, HIV protease, HIV integrase, Gag, Pol, gp160, Tat, Rev, Nef, Vpr, Vif, Vpu, RNA polymerase, GABA transaminase, Reverse transcriptase, DNA polymerase, prolactin, ACTH, ANP, insulin, PDE, AMPK, iNOS, HDAC1, HDAC2, HDAC3, HDAC4, HDAC5, HDAC6, HDAC7, HDAC8, HDAC9, HDAC10, HDAC11, lactase, amylase lysozyme, neuraminidase, invertase, chitinase, hyaluronidase, maltase, sucrase, phosphatase, phosphorylases, P, Histidine decarboxylase, PTEN, histone lysine demethylase (KDM), GCNS, PCAF, Hat1, ATF-2, Tip60, MOZ, MORF, HBO1, p300, CBP, SRC-1, SRC-3, ACTR, TIF-2, TAF1, TFIIIC, protein)-mannosyl-transferase 1 (POMT1), amyloid β and Tau, Apoptosis associated speck-like protein containing a CARD (ASC), and NACHT, LRR and PYD domains-containing protein 3 (NALP3).

### Certain Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, “cell penetrating peptide” or “CPP” refers to a peptide that facilitates delivery of a cargo, e.g., a therapeutic moiety (TM) into a cell. In embodiments, the CPP is cyclic, and is represented as “cCPP”. In embodiments, the cCPP is capable of directing a therapeutic moiety to penetrate the membrane of a cell. In embodiments, the cCPP delivers the therapeutic moiety to the cytosol of the cell. In embodiments, the cCPP delivers an antisense compound (AC) to a cellular location where a pre-mRNA is located.

As used herein, the term “endosomal escape vehicle” (EEV) refers to a cCPP that is conjugated by a chemical linkage (i.e., a covalent bond or non-covalent interaction) to a linker and/or an exocyclic peptide (EP). The EEV can be an EEV of Formula (B).

As used herein, the term “EEV-conjugate” refers to an endosomal escape vehicle defined herein conjugated by a chemical linkage (i.e., a covalent bond or non-covalent interaction) to a cargo. The cargo can be a therapeutic moiety (e.g., an oligonucleotide, peptide, or small molecule) that can be delivered into a cell by the EEV. The EEV-conjugate can be an EEV-conjugate of Formula (C).

As used herein, the term “exocyclic peptide” (EP) and “modulatory peptide” (MP) may be used interchangeably to refer to two or more amino acid residues linked by a peptide bond that can be conjugated to a cyclic cell penetrating peptide (cCPP) disclosed herein. The EP, when conjugated to a cyclic peptide disclosed herein, may alter the tissue distribution and/or retention of the compound. Typically, the EP comprises at least one positively charged amino acid residue, e.g., at least one lysine residue and/or at least one arginine residue. Non-limiting examples of EP are described herein. The EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV (SEQ ID NO: 42), the nucleoplasmin bipartite NLS with the sequence NLSKRPAAIKKAGQAKKKK (SEQ ID NO: 280), the c-myc nuclear localization sequence having the amino acid sequence PAAKRVKLD (SEQ ID NO: 281) or RQRRNELKRSF (SEQ ID NO: 282), the sequence RMRKFKNKGKDTAELRRRRVEVSVELRKAKKDEQILKRRNV (SEQ ID NO: 421) of the IBB domain from importin-alpha, the sequences VSRKRPRP (SEQ ID NO: 285) and PPKKARED (SEQ ID NO: 286) of the myoma T protein, the sequence PQPKKKPL (SEQ ID NO: 287) of human p53, the sequence SALIKKKKKMAP (SEQ ID NO: 288) of mouse c-abl IV, the sequences DRLRR (SEQ ID NO: 289) and PKQKKRK (SEQ ID NO: 290) of the influenza virus NS1, the sequence RKLKKKIKKL (SEQ ID NO: 291) of the Hepatitis virus delta antigen and the sequence REKKKFLKRR (SEQ ID NO: 292) of the mouse M×1 protein, the sequence KRKGDEVDGVDEVAKKKSKK (SEQ ID NO: 293) of the human poly(ADP-ribose) polymerase and the sequence RKCLQAGMNLEARKTKK (SEQ ID NO: 294) of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., an exocyclic peptide (EP) and a cargo, e.g., an oligonucleotide, peptide or small molecule) to the cyclic cell penetrating peptide (cCPP). The linker can comprise a natural or non-natural amino acid or polypeptide. The linker can be a synthetic compound containing two or more appropriate functional groups suitable to bind the cCPP to a cargo moiety, to thereby form the compounds disclosed herein. The linker can comprise a polyethylene glycol (PEG) moiety. The linker can comprise one or more amino acids. The cCPP may be covalently bound to a cargo via a linker.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins and the like.

As used herein, the term “contiguous” refers to two amino acids, which are connected by a covalent bond. For example, in the context of a representative cyclic cell penetrating peptide (cCPP) such as

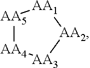

AA₁/AA₂, AA₂/AA₃, AA₃/AA₄, and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic cell penetrating peptides (cCPP) described herein have amino acids (e.g., arginine) incorporated therein through formation of one or more peptide bonds. The amino acids incorporated into the cCPP may be referred to residues or simply as an amino acid. Thus, arginine or an arginine residue refers to

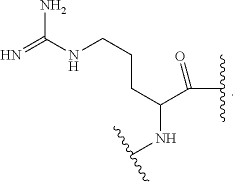 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

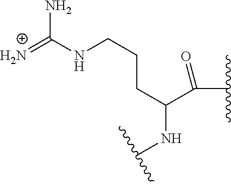 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. The chiral molecule can be an amino acid residue having a “D” and “L” enantiomer. Molecules without a chiral center, such as glycine, can be referred to as “achiral.”

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein.

As used herein “aromatic” refers to an unsaturated cyclic molecule having 4n+2π electrons, wherein n is any integer. The term “non-aromatic” refers to any unsaturated cyclic molecule which does not fall within the definition of aromatic.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where R^{a} and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NRgSO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “XY [symbol] ” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “XY [symbol] ” infers that when R³ is “XY”, the point of attachment bond is the same bond as the bond by which R³ is depicted as being bonded to CH₃.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The terms “inhibit”, “inhibiting” or “inhibition” refer to a decrease in an activity, expression, function or other biological parameter and can include, but does not require complete ablation of the activity, expression, function or other biological parameter. Inhibition can include, for example, at least about a 10% reduction in the activity, response, condition, or disease as compared to a control. In embodiments, expression, activity or function of a gene or protein is decreased by a statistically significant amount. In embodiments, activity or function is decreased by at least about 10%, about 20%, about 30%, about 40%, about 50%, and up to about 60%, about 70%, about 80%, about 90% or about 100% In embodiments, the expression, activity or function of IRF-5 is inhibited.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to a carrier suitable for administration to a patient. A pharmaceutically acceptable carrier can be a sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In embodiments, a dose may be administered in two or more boluses, tablets, or injections. In embodiments, where subcutaneous administration is desired, the desired dose requires a volume not easily accommodated by a single injection. In such embodiments, two or more injections may be used to achieve the desired dose. In embodiments, a dose may be administered in two or more injections to reduce injection site reaction in a patient.

As used herein, the term “dosage unit” refers to a form in which a pharmaceutical agent is provided. In embodiments, a dosage unit is a vial that includes lyophilized antisense oligonucleotide. In embodiments, a dosage unit is a vial that includes reconstituted antisense oligonucleotide.

The terms “modulate”, “modulating” and “modulation” refer to a perturbation of expression, function or activity when compared to the level of expression, function or activity prior to modulation. Modulation can include an increase (stimulation or induction) or a decrease (inhibition or reduction) in expression, function or activity. In embodiments, the compound disclosed herein includes a therapeutic moiety (TM) that decreases TRF-5 expression, function and/or activity. In embodiments, TRF-5 activity is modulated by modulating TRF-5 expression.

“Amino acid” refers to an organic compound that includes an amino group and a carboxylic acid group and has the general formula

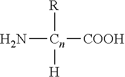

where R can be any organic group. An amino acid may be a naturally occurring amino acid or non-naturally occurring amino acid. An amino acid may be a proteogenic amino acid or a non-proteogenic amino acid. An amino acid can be an L-amino acid or a D-amino acid. The term “amino acid side chain” or “side chain” refers to the characterizing substituent (“R”) bound to the α-carbon of a natural or non-natural α-amino acid. An amino acid may be incorporated into a polypeptide via a peptide bond.

The term “antigen-binding domain” as used herein refers to a polypeptide that binds to an antigen. The antigen-binding domain may be an antibody, an antigen-binding fragment, or an antibody mimetic.

The term “antigen-binding fragment” or “antigen-binding antibody fragment” are used interchangeable herein and refer to a polypeptide fragment that contains at least one complementarity-determining region (CDR) of an immunoglobulin heavy and/or light chain that binds to at least one epitope of the antigen of interest. The CDR may be derived from a human immunoglobulin or a camelid immunoglobulin. In this regard, an antigen-binding fragment of the herein described antibodies may comprise 1, 2, 3, 4, 5, or all 6 CDRs of a variable heavy chain (VH) and variable light chain (VL) sequence from antibodies that specifically bind to a target molecule. Antigen-binding fragments include proteins that comprise a portion of a full length antibody, generally the antigen binding or variable region thereof, such as Fab, F(ab′)2, Fab′, Fv fragments, minibodies, diabodies, single domain antibodies (dAb), single-chain variable fragments (scFv), multispecific antibodies formed from antibody fragments, and any other modified configuration of the immunoglobulin molecule that comprises an antigen-binding site or fragment of the required specificity.

The term “antigen-binding domain” as used herein refers to a polypeptide that binds to an antigen. The antigen-binding domain me be an antibody, an antigen-binding fragment, or an antibody mimetic.

The term “antibody” as used herein refers to a molecule that contains at least one antigen binding site that immunospecifically binds to a particular antigen target of interest. The term “antibody” thus includes but is not limited to a full-length antibody and/or its variants, a fragment thereof, peptibodies and variants thereof, monoclonal antibodies (including full-length monoclonal antibodies), polyclonal antibodies, multispecific antibodies (for example, bispecific antibodies) formed from at least two intact antibodies, human antibodies, humanized antibodies, and antibody mimetics that mimic the structure and/or function of an antibody or a specified fragment or portion thereof, including single chain antibodies and fragments thereof. Binding of an antibody to a target can cause a variety of effects, such as but not limited to where such binding modulates, decreases, increases, antagonizes, agonizes, mitigates, alleviates, blocks, inhibits, abrogates and/or interferes with at least one target activity or binding, or with receptor activity or binding, in vitro, in situ, and/or in vivo. An antibody of the present disclosure encompasses antibody fragments capable of binding to a particular antigen target of interest, including but not limited to Fab, Fab′, F(ab′)₂, pFc′, Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody formed from antibody fragments. The antibody may be of any type, any class, or any subclass.

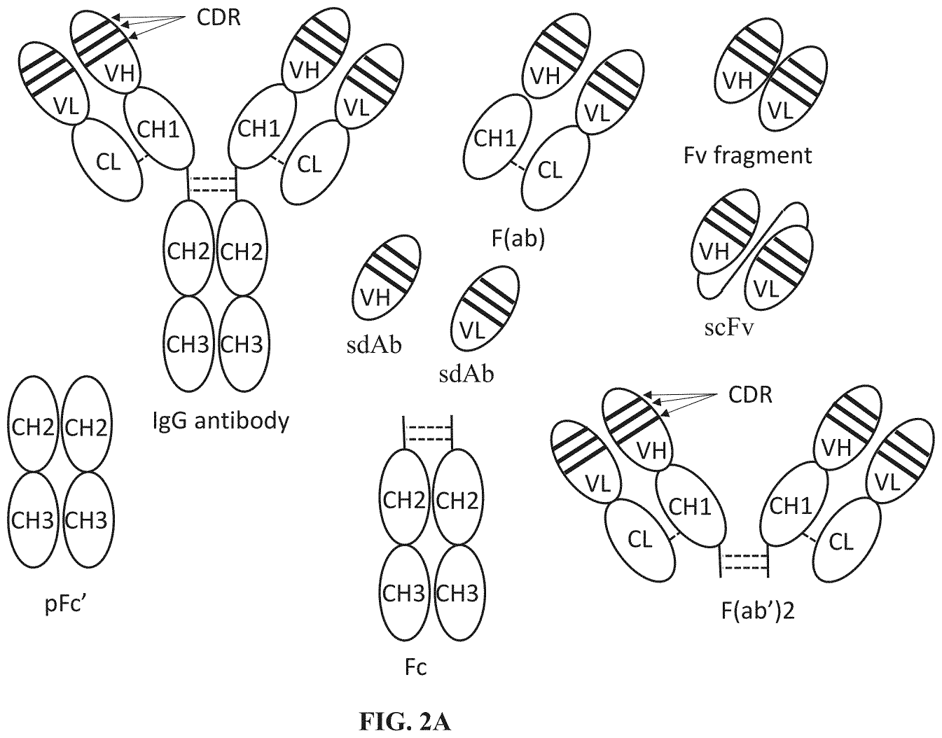

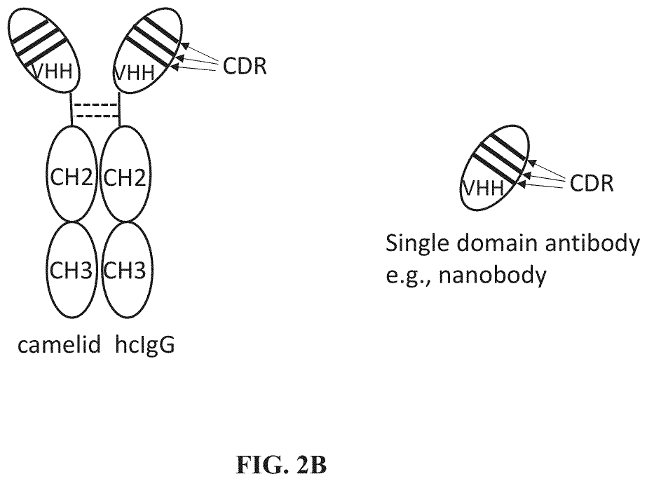

When the antibody is a human or mouse antibody, the type may include, for example, IgG, IgE, IgM, IgD, IgA and IgY, and the class may include, for example, IgG1, IgG2, IgG3, IgG4, IgA1 and IgA2. When the antibody is an IgG antibody, the antibody includes a two light chains and two heavy chains. The light chains include two variable regions (VL) and two conserved regions (CL). The heavy chain includes two variable regions (VH) and three conserved regions (CH1, CH2, CH3). Each of the heavy chains associate with a light chain by virtue of interchain disulfide bonds between the heavy and light chain to form two heterodimeric proteins or polypeptides (i.e., a protein comprised of two heterologous polypeptide chains). The two heterodimeric proteins then associate by virtue of additional interchain disulfide bonds between the heavy chains to form an Ig molecule (See FIG. 2A).

When the antibody is a camelid antibody, the type may include, for example, camelid heavy chain IgG (hcIgG), camelid single N-terminal variable domain heavy chain (VHH) region, and single domain antibody comprising the VHH (See FIG. 2B).

The term “antigen-binding fragment” as used herein refers to a polypeptide fragment that contains at least one complementarity-determining region (CDR) of an immunoglobulin heavy and/or light chain that binds to at least one epitope of the antigen of interest. In this regard, an antigen-binding fragment of the herein described antibodies may comprise 1, 2, 3, 4, 5, or all 6 CDRs of a variable heavy chain (VH) and variable light chain (VL) sequence from antibodies that specifically bind to a target molecule. The antigen-binding fragment of the herein described camelid antibodies may comprise 1, 2, or 3 of the CDRs of a camelid VHH region. The antigen-binding fragment of the herein described camelid antibodies may be a single domain antibody (VHH). Antigen-binding fragments include proteins that comprise a portion of a full length antibody, generally the antigen binding or variable region thereof, such as Fab, F(ab′)2, Fab′, Fv fragments, minibodies, single domain antibodies (dAb), single-chain variable fragments (scFv), divalent scFv such as diabodies, multispecific antibodies formed from antibody fragments, and any other modified configuration of the immunoglobulin molecule that comprises an antigen-binding site or fragment of the required specificity (See FIG. 2A/B).

The term “antibody mimetic” refers to a polypeptide that can specifically bind an antigen but is not structurally related to an antibody. Examples of antibody mimetics include monobodies, affibody molecules (constructed on a scaffold of the Z-domain of Protein A, See, Nygren, FEBS J. (2008), 275 (11): 2668-76), affilins (constructed on a scaffold of gamma-B crystalline or ubiquitin, See Ebersbach H et al., J. Mol. Biol. (2007), 372 (1): 172-85), affimers (constructed on a Crystatin scaffold, See Johnson A et al., Anal. Chem. (2012), 84 (15): 6553-60), affitins (constructed on a Sac7d from S. acidocaldarius scaffold, See Krehenbrink M et al., J. Mol. Biol. (2008), 383 (5): 1058-68), alphabodies (constructed on a triple helix coiled coil scaffold, See Desmet, J et al., Nature Communications (2014), 5: 5237), anticalins (constructs on scaffold of lipocalins, See Skerra A., FEBS J. (2008), 275 (11): 2677-83), avimers (constructed on scaffolds of various membrane receptors, See Silverman J. et al., Nat. Biotechnol. (2005), 23 (12): 1556-61), DARPins (constructed on scaffolds of ankyrin repeat motifs, See Stumpp et al., Drug Discov. Today (2008), 3 (15-16): 695-701), fynomers (constructed on a scaffold of the SH3 domain of Fyn, See Grabulovski et al., J Biol Chem. (2007), 282 (5): 3196-3204), Kunitz domain peptides (constructed on scaffolds of the Kunitz domains of various protease inhibitors, See Nixon et. al., Curr. Opin. Drug. Discov. Dev. (2006), 9 (2): 261-8), and monobodies (constructed on scaffolds of type III domain of fibronectin, See Koide et al (2007).

The term “monobody” refers to a synthetic binding protein constructed using a fibronectin type III domain (FN3) as a molecular scaffold.

The term “minibody” refers to a CH3 domain fused or linked to an antigen-binding fragment (e.g., a CH3 domain fused or linked to an scFv, a domain antibody, etc.). In embodiments, the term “Mb” signifies a CH3 single domain. In other embodiments, a CH3 domain signifies a minibody. (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The term “F(ab)” refers to two of the protein fragments resulting from proteolytic cleavage of IgG molecules by the enzyme papain. Each F(ab) comprises a covalent heterodimer of the VH chain and VL chain and includes an intact antigen-binding site. Each F(ab) is a monovalent antigen-binding fragment.

The term “F(ab′)2” refers to a protein fragment of IgG generated by proteolytic cleavage by the enzyme pepsin. Each F(ab′)2 fragment comprises two F(ab′) fragments linked by disulfide bonds in the hinge region and is therefore a bivalent antigen-binding fragment. The term “Fab′” refers to a fragment derived from F(ab′)2 and may contain a small portion of the Fc. Each Fab′ fragment is a monovalent antigen-binding fragment.

An “Fv fragment” refers to a non-covalent VH::VL heterodimer which includes an antigen-binding site that retains much of the antigen recognition and binding capabilities of the native antibody molecule, but lacks the CH1 and CL domains contained within a Fab. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

“Fc region” or “Fc domain” refers to a polypeptide sequence corresponding to or derived from the portion of an antibody that is capable of binding to Fc receptors on cells and/or the C1q component or complement, thereby mediating the effector function of an antibody. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc region is a homodimeric protein comprising two polypeptides that are associated by disulfide bonds, and each comprising a hinge region, a CH2 domain, and a CH3 domain. However, more recently the term has been applied to the single chain monomer component consisting of CH3, CH2, and at least a portion of the hinge sufficient to form a disulfide-linked dimer with a second such chain. As such, and depending on the context, use of the terms “Fc region” or “Fc domain” will refer herein to either the dimeric form or the individual monomers that associate to form the dimeric protein. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc domain includes variants of naturally occurring sequences.

A pFc′ fragment refers to an Fc region that is not covalently coupled.

A “single domain antibody” (sdAb) refers to an antibody fragment comprising a single monomeric heavy chain variable domain. In embodiments, where the antibody fragment is from a camelid heavy chain IgG, the variable domain may be the VHH.

The term “antigen” refers to a molecule or a portion of a molecule capable of being bound by an antibody or an antigen-binding fragment thereof and additionally capable of being used in an animal to produce antibodies capable of binding to an epitope of that antigen.

Antigens include, but are not limited to, proteins, polysaccharides, lipids, or glycolipids. In embodiments, an antigen is an antigen of an infectious agent. In embodiments, the antigen is an extracellular antigen. In embodiments, the antigen is a cell surface antigen. In embodiments, the antigen is an intracellular antigen. An antigen may have one or more epitopes.

The term “epitope” refers to a region of an antigen that is bound by an antibody. Epitope determinants can include chemically active surface groupings of molecules such as amino acids, sugar side chains, phosphoryl, or sulfonyl and may have specific three-dimensional structural characteristics, and/or specific charge characteristics.

The terms “light chain variable region” (also referred to as “light chain variable domain” or “VL”) and “heavy chain variable region” (also referred to as “heavy chain variable domain” or “VH”) refer to the variable binding region from an antibody light and heavy chain, respectively.

The variable binding regions are made up of discrete, well-defined sub-regions known as “complementarity determining regions” (CDRs) and “framework regions” (FRs).

The term “immunoglobulin light chain constant region” (also referred to as “light chain constant region” or “CL”) is a constant region from an antibody light chain.

The term “immunoglobulin heavy chain constant region” (also referred to as “heavy chain constant region” or “CH”) refers to the constant region from the antibody heavy chain. The CH is further divisible, depending on the antibody isotype into CH1, CH2, and CH3 (IgA, IgD, IgG), or CH1, CH2, CH3, and CH4 domains (IgE, IgM).

The term “single-chain variable fragment (scFv) refers to fusion between a VH and VL. Generally. The N-terminus of the VH and the C-terminus of the VL or the N-terminus of the VL and the C-terminus of the VH are coupled through a linker peptide.

The term “variable region of heavy chain only” or “variable region of hcIgG” (VHH) refers to the variable region of an hcIgG such as those from camelids. A VHH includes 3 CDRs.

Divalent single chain variable fragment (di-scFv) refers to the association of two or more scFvs either through covalent bonds or non-covalent means such as dimerization. A diabody is a dimer of two scFv where the scFv comprise a VH and VL linked by a peptide linker that is too short allow for intramolecular association.

As used herein, the term “complementarity determining region” or “CDR” refer to an immunoglobulin (antibody) molecule. There are three CDRs per variable domain: CDR1, CDR2 and CDR3 in the variable domain of the light chain and CDR1, CDR2 and CDR3 in the variable domain of the heavy chain. In camelid antibodies and antigen binding fragments thereof, there are three CDRs per VHH.

As used herein “active portion” or “active portion thereof” refers to a fragment of a polypeptide that retains the function of the polypeptide. Functions include but are not limited to, binding and or/enzymatic activity. The binding affinity of a active portion need not be the same as the full polypeptide.

The term “specifically binds” refers to the ability of an antibody or antigen-binding fragment thereof to bind a target antigen with a binding affinity (Ka) of at least 10⁵ M⁻¹ while not significantly binding other components or antigens present in a mixture.

Binding affinity (Ka) refers to an equilibrium association of a particular interaction expressed in the units of 1/M or M⁻¹. Antibodies or antigen-binding antibody fragments thereof can be classified as “high affinity” antibodies or antigen-binding fragments thereof and “low affinity” antibodies or antigen-binding fragments thereof “High affinity” antibodies or antigen-binding fragments thereof refer to those antibodies or antigen-binding fragments thereof with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” antibodies or antigen-binding fragments thereof refer to those antibodies or antigen-binding fragments thereof with a Ka of up to 10⁷ M⁻¹, up to 10⁶ M⁻¹, up to 10⁵ M⁻¹. Alternatively, affinity can be defined as an equilibrium dissociation constant (Kd) of a particular binding interaction with units of M (e.g., 10⁻⁵ M to 10⁻¹³, or about 500 nM, about 300 nM, about 250 nM, about 200 nM, about 150 nM, about 100 nM, about 50 nM, about 25 nM, about 10 nM, or about 5 nM). Affinities of binding domain polypeptides and single chain polypeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

The term “infectious agent” refers to a pathogenic microorganism, including, but not limited to, bacteria, viruses, fungi, or intracellular or extracellular parasites.

The term “infection” refers to a pathological process caused by the invasion of normally sterile tissue or fluid by an infectious agent including, but not limited to, infection by bacteria, viruses, fungi, and/or parasites. An infection can be local or systemic. A subject suffering from an infection can suffer from more than one source of infection simultaneously. For example, a subject can suffer from a bacterial infection and viral infection; a viral infection and fungal infection; a bacterial and fungal infection; a bacterial infection, a fungal infection and a viral infection; or a mixture of one or more infections. A subject can suffer from one or more bacterial infections, one or more viral infections, one or more fungal infections and/or one or more parasitic infections, simultaneously or sequentially.

The term “variant” or “variants” as used herein refers to a polynucleotide or polypeptide with a sequence differing from that of a reference polynucleotide or polypeptide, but retaining essential properties of the parental polynucleotide or polypeptide. Generally, variant polynucleotide or polypeptide sequences are overall closely similar, and, in many regions, identical to the parental polynucleotide or polypeptide. For instance, a variant polynucleotide or polypeptide may exhibit at least about 70%, at least about 80%, at least about 90%, at least about 91%, at least about 92%, at least about 93%, at least about 94%, at least about 95%, at least about 96%, at least about 97%, at least about 98% at least about 99%, or at least about 99.5% sequence identity compared to the parental polynucleotide or polypeptide.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage sequence identity is calculated by determining the number of positions at which the identical nucleic acid base or amino acid residue occurs in both sequences to yield the number of identical positions. The number of identical positions is then divided by the total number of positions in the comparison window and multiplied by 100 to yield the percentage of sequence identity. Percentage of sequence identity is determined by comparing two optimally aligned sequences over a comparison window. The comparison window for polynucleotide sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least 80%, at least 85%, at least 90%, at least 95%, at least 96%, at least 97%, at least 98%, or at least 99% sequence identity relative to each other.

The term “substantially identical” refers to a polypeptide sequence that contains a sufficient number of identical amino acids to a second polypeptide sequence such that the first and second polypeptide sequence have similar activity. Polypeptides that are substantially identical are at least about 80%, at least about 85%, at least about 90%, at least about 95%, at least about 96%, at least about 97%, at least about 98%, at least about 99%, or at least about 100% identical in amino acid sequence.

The term “polynucleotide” as referred to herein means single-stranded or double-stranded nucleic acid polymers. In certain embodiments, the nucleotides comprising the polynucleotide can be RNA or DNA or a modified form of either type of nucleotide, such as a modified messenger RNA. Said modifications may include, but are not limited to, base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

As used herein, a “polypeptide” or “protein” refers to a single, linear, and contiguous arrangement of covalently linked amino acids. Polypeptides can form one or more intrachain disulfide bonds. The terms polypeptide and protein also encompass embodiments where two polypeptide chains link together in a non-linear fashion, such as via an interchain disulfide bond. Herein, a protein or polypeptide may be an antibody or an antigen-binding fragment of an antibody.

All publications, patents and patent applications mentioned in the specification are indicative of the level of skill of those skilled in the art to which this invention pertains. All publications, patents and patent applications are herein incorporated by reference to the same extent as if each individual publication or patent application was specifically and individually indicated to be incorporated by reference.

### EXAMPLES

Data illustrating various embodiments of the present disclosure are provided in the figures submitted herewith.

### Example 1. Evaluation of Various K19-DM, K19-DM-EEV Constructs

Various K19-DM and K19-DM-EEV constructs (DM=degradation moiety) were produced and evaluated for their ability to degrade KRas.

Production of KRAS Target Protein

DNA sequences for human KRas-WT and its variants KRas-G12D), KRas G13D; and human HRas-WT and its variants HRas-G12D and HRas-G12V were codon optimized, synthesized, and subcloned into vector pGEX-4T-1 with N-terminal GST+Flag tag (Table 21 has the full amino acid sequences and DNA sequences). The variants were expressed, for example, culturing at 37° C. and 200 rpm following by induction with IPTG after OD₆₀₀ reach ˜1.2 and allowing expression at 30° C. for 4 hours. Proteins were purified using GSH agarose. The protein purity and molecular weight were determined by standard SDS-PAGE along with identity conformation by Western blot (data not shown).

Purified GST-KRas-WT, GST-KRas-G12D, and GST-KRas-G12V were nucleotide exchanged. Briefly, the proteins were exchanged into nucleotide exchange buffer (25 mM Tris, 0.1 mM ZnCl₂, 200 mM (NH₄)₂SO₄). After exchange, alkaline phosphatase agarose beads (3 units/mg KRas, Sigma-Aldrich) and 10 eq. GPPNHP/GTP/GTPγS (Sigma-Aldrich) were added, and the solution was incubated at RT with gentle mixing for 3 h. After 3 h, the solution was centrifuged to remove the alkaline phosphatase beads and to the supernatant was added 20 mM MgCl₂ 5 eq. of GPPNHP/GTP/GTPγS and incubated for an additional 1 h at RT with gentle mixing. Nucleotide exchanged protein was desalted and exchanged into storage buffer (20 mM HEPES, 150 mM NaCl, 5 mM MgCl₂, 1 mM TCEP, pH 7.4), aliquoted and frozen.

The extent of nucleotide exchange was assessed by determining the binding affinity of GST-Raf-RBD using biolayer interferometry. 250 nM GST-Raf-RBD was immobilized on streptavidin biosensors (ForteBio) in 10× KB buffer (ForteBio) before association with GDP- or nucleotide-exchanged various KRas proteins at concentrations of 625 nM, 125 nM, 25 nM, or 5 nM in 10× KB buffer. The extent of exchange was determined by comparison to known binding affinity of KRas-Raf-RBD. The observed Ras/Raf binding affinity of ˜0.03 μM is in good agreement with previously reported literature value of 0.02 μM for Ras-WT-GPPNHP/Raf-RBD interaction (Block et al., Nat. Struct. Biol. (1996), 3(3), 244-251).

### Production of KRAS-Binding Protein DARPIN (K19)

Plasmids that included KRAS binding K19 or variants of K19 were prepared via de novo gene synthesis and cloned into pET-16b vector and recombinantly expressed in E. coli (Table 21 shows the full amino acid sequence and DNA sequence). For expression, cultures were inoculated and allowed to grow at 37° C. and 250 RPM until the OD600 reached ˜ 0.6. Expression was induced with 0.5 mM IPTG and protein allowed to express for 4 hours at 30° C. and 250 RPM. Cells were lysed and purified using Ni-NTA agarose. Protein purity was determined by SDS-PAGE to be >90%.

### Production of K19-IgG1-Fc Fusion

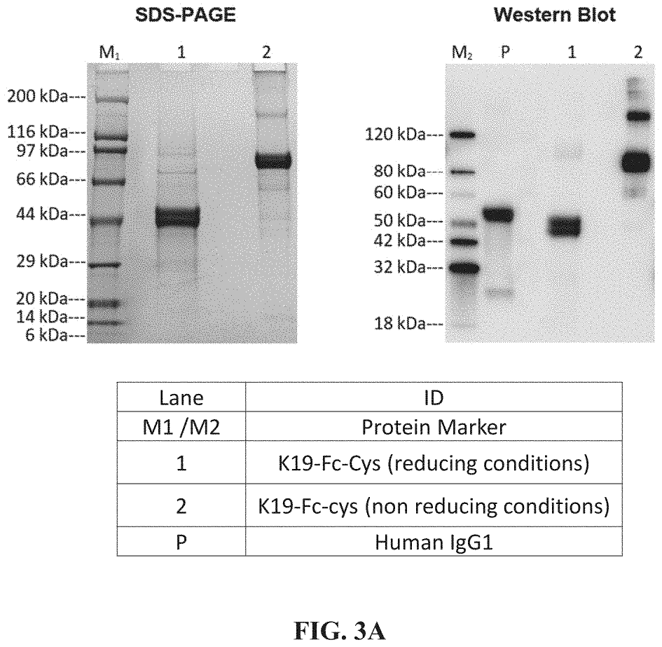

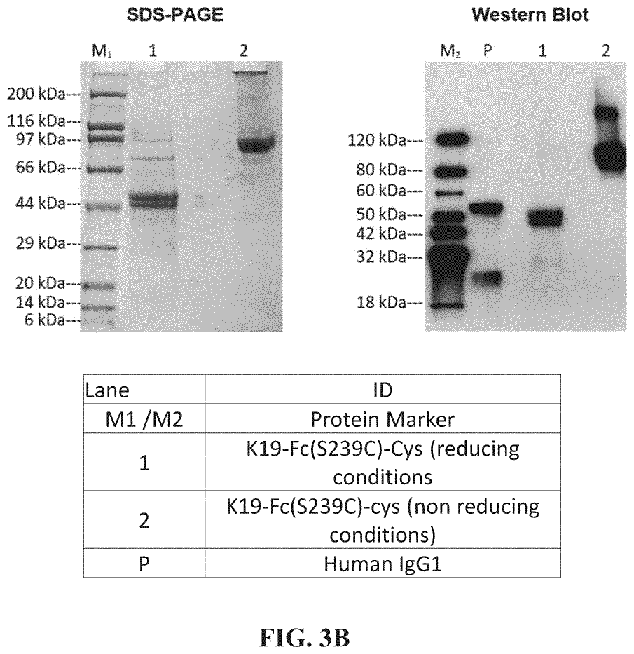

K19 was fused with human IgG1-Fc(N297A) (hFc(N297); Fc(N297). For mammalian cell expression, K19-Fc(N297A)-Cys and K19-Fc(S293C) fusion sequences were designed, optimized, synthesized and subcloned into pcDNA3.4 vector and transiently co-transfected into suspension Expi293F cell cultures. The transfected cells were grown in Erlenmeyer Flasks (Corning Inc.) at 37° C. with 8% CO₂ on an orbital shaker. The cell culture supernatants collected on day six were used for purification via affinity MabSelect SuRe LX purification column. The purified protein was analyzed by SDS-PAGE, Western blot (primary antibody Goat Anti-Human IgG-HRP) and to determine the molecular weight and purity FIG. 3A-B.

TABLE 21
Amino acid and DNA sequences of constructs in Example 1

| | SEQ | | SEQ | |
|---|---|---|---|---|
| | ID | | ID | |
| Construct | NO: | Sequence | NO: | Sequence |
| GST-KRas- | 422 | MSPILGYWKIKGLVQPTRLLLEYLEE | 429 | GGATCCGACTACAAGGACGATGACG |
| WT | | KYEEHLYERDEGDKWRNKKFELGLEF | | ATAAAATGACCGAGTATAAACTGGT |
| | | PNLPYYIDGDVKLTQSMAIIRYIADK | | GGTGGTGGGTGCGGGTGGCGTTGGT |
| | | HNMLGGCPKERAEISMLEGAVLDIRY | | AAAAGCGCGCTGACCATCCAGCTGA |
| | | GVSRIAYSKDFETLKVDFLSKLPEML | | TTCAAAACCACTTCGTGGACGAGTA |
| | | KMFEDRLCHKTYLNGDHVTHPDFMLY | | CGATCCGACCATCGAAGACAGCTAT |
| | | DALDVVLYMDPMCLDAFPKLVCFKKR | | CGTAAGCAGGTTGTGATCGATGGCG |
| | | IEAIPQIDKYLKSSKYIAWPLQGWQA | | AGACCTGCCTGCTGGACATTCTGGA |
| | | TFGGGDHPPKSDLVPRGSDYKDDDDK | | TACCGCGGGCCAGGAAGAGTACAGC |
| | | MTEYKLVVVGAGGVGKSALTIQLIQN | | GCGATGCGTGACCAATATATGCGTA |
| | | HFVDEYDPTIEDSYRKQVVIDGETCL | | CCGGTGAAGGCTTCCTGTGCGTTTT |
| | | LDILDTAGQEEYSAMRDQYMRTGEGF | | TGCGATTAACAACACCAAAAGCTTT |
| | | LCVFAINNTKSFEDIHHYREQIKRVK | | GAGGATATCCACCACTACCGTGAAC |
| | | DSEDVPMVLVGNKCDLPSRTVDTKQA | | AAATTAAGCGTGTTAAAGACAGCGA |
| | | QDLARSYGIPFIETSAKTRQRVEDAF | | GGATGTGCCGATGGTTCTGGTGGGT |
| | | YTLVREIRQYRLKKISKEEKTPGCVK | | AACAAATGCGACCTGCCGAGCCGTA |
| | | IKKC | | CCGTGGACACCAAGCAGGCGCAAGA |
| | | | | TCTGGCGCGTAGCTATGGCATCCCG |
| | | | | TTCATTGAAACCAGCGCGAAAACCC |
| | | | | GTCAGCGTGTTGAGGATGCGTTTTA |
| | | | | CACCCTGGTGCGTGAAATCCGTCAA |
| | | | | TATCGTCTGAAGAAAATTAGCAAAG |
| | | | | AGGAGAAAACCCCGGGTTGCGTTAA |
| | | | | GATCAAGAAATGCTAATGACTCGAG |
| GST-KRas- | 423 | MSPILGYWKIKGLVQPTRLLLEYLEE | 430 | GGATCCGACTACAAGGACGATGACG |
| G12V | | KYEEHLYERDEGDKWRNKKFELGLEF | | ATAAAATGACCGAGTATAAACTGGT |
| | | PNLPYYIDGDVKLTQSMAIIRYIADK | | GGTGGTGGGTGCGGTTGGCGTTGGT |
| | | HNMLGGCPKERAEISMLEGAVLDIRY | | AAAAGCGCGCTGACCATCCAGCTGA |
| | | GVSRIAYSKDFETLKVDFLSKLPEML | | TTCAAAACCACTTCGTGGACGAGTA |
| | | KMFEDRLCHKTYLNGDHVTHPDFMLY | | CGATCCGACCATCGAAGACAGCTAT |
| | | DALDVVLYMDPMCLDAFPKLVCFKKR | | CGTAAGCAGGTTGTGATCGATGGCG |
| | | IEAIPQIDKYLKSSKYIAWPLQGWQA | | AGACCTGCCTGCTGGACATTCTGGA |
| | | TFGGGDHPPKSDLVPRGSDYKDDDDK | | TACCGCGGGCCAGGAAGAGTACAGC |
| | | MTEYKLVVVGAVGVGKSALTIQLIQN | | GCGATGCGTGACCAATATATGCGTA |
| | | HFVDEYDPTIEDSYRKQVVIDGETCL | | CCGGTGAAGGCTTCCTGTGCGTTTT |
| | | LDILDTAGQEEYSAMRDQYMRTGEGF | | TGCGATTAACAACACCAAAAGCTTT |
| | | LCVFAINNTKSFEDIHHYREQIKRVK | | GAGGATATCCACCACTACCGTGAAC |
| | | DSEDVPMVLVGNKCDLPSRTVDTKQA | | AAATTAAGCGTGTTAAAGACAGCGA |
| | | QDLARSYGIPFIETSAKTRQRVEDAF | | GGATGTGCCGATGGTTCTGGTGGGT |
| | | YTLVREIRQYRLKKISKEEKTPGCVK | | AACAAATGCGACCTGCCGAGCCGTA |
| | | IKKC | | CCGTGGACACCAAGCAGGCGCAAGA |
| | | | | TCTGGCGCGTAGCTATGGCATCCCG |
| | | | | TTCATTGAAACCAGCGCGAAAACCC |
| | | | | GTCAGCGTGTTGAGGATGCGTTTTA |
| | | | | CACCCTGGTGCGTGAAATCCGTCAA |
| | | | | TATCGTCTGAAGAAAATTAGCAAAG |
| | | | | AGGAGAAAACCCCGGGTTGCGTTAA |
| | | | | GATCAAGAAATGCTAATGA |
| | 424 | MSPILGYWKIKGLVQPTRLLLEYLEE | 431 | GGATCCGACTACAAGGACGATGACG |
| | | KYEEHLYERDEGDKWRNKKFELGLEF | | ATAAAATGACCGAGTATAAACTGGT |
| | | PNLPYYIDGDVKLTQSMAIIRYIADK | | GGTGGTGGGTGCGGATGGCGTTGGT |
| | | HNMLGGCPKERAEISMLEGAVLDIRY | | AAAAGCGCGCTGACCATCCAGCTGA |
| | | GVSRIAYSKDFETLKVDFLSKLPEML | | TTCAAAACCACTTCGTGGACGAGTA |
| | | KMFEDRLCHKTYLNGDHVTHPDFMLY | | CGATCCGACCATCGAAGACAGCTAT |
| | | DALDVVLYMDPMCLDAFPKLVCFKKR | | CGTAAGCAGGTTGTGATCGATGGCG |
| | | IEAIPQIDKYLKSSKYIAWPLQGWQA | | AGACCTGCCTGCTGGACATTCTGGA |
| | | TFGGGDHPPKSDLVPRGSDYKDDDDK | | TACCGCGGGCCAGGAAGAGTACAGC |
| | | MTEYKLVVVGADGVGKSALTIQLIQN | | GCGATGCGTGACCAATATATGCGTA |
| | | HFVDEYDPTIEDSYRKQVVIDGETCL | | CCGGTGAAGGCTTCCTGTGCGTTTT |
| | | LDILDTAGQEEYSAMRDQYMRTGEGF | | TGCGATTAACAACACCAAAAGCTTT |
| | | LCVFAINNTKSFEDIHHYREQIKRVK | | GAGGATATCCACCACTACCGTGAAC |
| | | DSEDVPMVLVGNKCDLPSRTVDTKQA | | AAATTAAGCGTGTTAAAGACAGCGA |
| | | QDLARSYGIPFIETSAKTRQRVEDAF | | GGATGTGCCGATGGTTCTGGTGGGT |
| | | YTLVREIRQYRLKKISKEEKTPGCVK | | AACAAATGCGACCTGCCGAGCCGTA |
| | | IKKC | | CCGTGGACACCAAGCAGGCGCAAGA |
| | | | | TCTGGCGCGTAGCTATGGCATCCCG |
| | | | | TTCATTGAAACCAGCGCGAAAACCC |
| | | | | GTCAGCGTGTTGAGGATGCGTTTTA |
| | | | | CACCCTGGTGCGTGAAATCCGTCAA |
| | | | | TATCGTCTGAAGAAAATTAGCAAAG |
| | | | | AGGAGAAAACCCCGGGTTGCGTTAA |
| | | | | GATCAAGAAATGCTAATGACTCGAG |
| | 425 | MSPILGYWKIKGLVQPTRLLLEYLEE | 432 | GGATCCGACTACAAGGACGATGACG |
| | | KYEEHLYERDEGDKWRNKKFELGLEF | | ATAAAATGACCGAGTATAAACTGGT |
| | | PNLPYYIDGDVKLTQSMAIIRYIADK | | GGTGGTGGGTGCGGGTGATGTTGGT |
| | | HNMLGGCPKERAEISMLEGAVLDIRY | | AAAAGCGCGCTGACCATCCAGCTGA |
| | | GVSRIAYSKDFETLKVDFLSKLPEML | | TTCAAAACCACTTCGTGGACGAGTA |
| | | KMFEDRLCHKTYLNGDHVTHPDFMLY | | CGATCCGACCATCGAAGACAGCTAT |
| | | DALDVVLYMDPMCLDAFPKLVCFKKR | | CGTAAGCAGGTTGTGATCGATGGCG |
| | | IEAIPQIDKYLKSSKYIAWPLQGWQA | | AGACCTGCCTGCTGGACATTCTGGA |
| | | TFGGGDHPPKSDLVPRGSDYKDDDDK | | TACCGCGGGCCAGGAAGAGTACAGC |
| | | MTEYKLVVVGAGDVGKSALTIQLIQN | | GCGATGCGTGACCAATATATGCGTA |
| | | HFVDEYDPTIEDSYRKQVVIDGETCL | | CCGGTGAAGGCTTCCTGTGCGTTTT |
| | | LDILDTAGQEEYSAMRDQYMRTGEGF | | TGCGATTAACAACACCAAAAGCTTT |
| | | LCVFAINNTKSFEDIHHYREQIKRVK | | GAGGATATCCACCACTACCGTGAAC |
| | | DSEDVPMVLVGNKCDLPSRTVDTKQA | | AAATTAAGCGTGTTAAAGACAGCGA |
| | | QDLARSYGIPFIETSAKTRQRVEDAF | | GGATGTGCCGATGGTTCTGGTGGGT |
| | | YTLVREIRQYRLKKISKEEKTPGCVK | | AACAAATGCGACCTGCCGAGCCGTA |
| | | IKK | | CCGTGGACACCAAGCAGGCGCAAGA |
| | | | | TCTGGCGCGTAGCTATGGCATCCCG |
| | | | | TTCATTGAAACCAGCGCGAAAACCC |
| | | | | GTCAGCGTGTTGAGGATGCGTTTTA |
| | | | | CACCCTGGTGCGTGAAATCCGTCAA |
| | | | | TATCGTCTGAAGAAAATTAGCAAAG |
| | | | | AGGAGAAAACCCCGGGTTGCGTTAA |
| | | | | GATCAAGAAATGCTAATGACTCGAG |
| | 426 | MSPILGYWKIKGLVQPTRLLLEYLEE | 433 | GGATCCGACTACAAGGACGATGACG |
| | | KYEEHLYERDEGDKWRNKKFELGLEF | | ATAAAATGACCGAGTATAAACTGGT |
| | | PNLPYYIDGDVKLTQSMAIIRYIADK | | GGTGGTGGGTGCGGGTGGCGTTGGT |
| | | HNMLGGCPKERAEISMLEGAVLDIRY | | AAAAGCGCGCTGACCATCCAGCTGA |
| | | GVSRIAYSKDFETLKVDFLSKLPEML | | TTCAAAACCACTTCGTGGACGAGTA |
| | | KMFEDRLCHKTYLNGDHVTHPDFMLY | | CGATCCGACCATCGAAGACAGCTAT |
| | | DALDVVLYMDPMCLDAFPKLVCFKKR | | CGTAAGCAGGTTGTGATCGATGGCG |
| | | IEAIPQIDKYLKSSKYIAWPLQGWQA | | AGACCTGCCTGCTGGACATTCTGGA |
| | | TFGGGDHPPKSDLVPRGSDYKDDDDK | | TACCGCGGGCCAGGAAGAGTACAGC |
| | | MTEYKLVVVGAGGVGKSALTIQLIQN | | GCGATGCGTGATCAATATATGCGTA |
| | | HFVDEYDPTIEDSYRKQVVIDGETCL | | CCGGTGAAGGCTTCCTGTGCGTTTT |
| | | LDILDTAGQEEYSAMRDQYMRTGEGF | | TGCGATTAACAACACCAAAAGCTTT |
| | | LCVFAINNTKSFEDIHQYREQIKRVK | | GAGGACATCCACCAGTACCGTGAAC |
| | | DSDDVPMVLVGNKCDLAARTVESRQA | | AAATTAAGCGTGTTAAAGATAGCGA |
| | | QDLARSYGIPYIETSAKTRQGVEDAF | | CGATGTGCCGATGGTTCTGGTGGGT |
| | | YTLVREIRQHKLRKLNPPDESGPGCM | | AACAAGTGCGACCTGGCGGCGCGTA |
| | | SCKCVLS | | CCGTGGAGAGCCGTCAGGCGCAAGA |
| | | | | TCTGGCGCGTAGCTACGGTATCCCG |
| | | | | TATATTGAAACCAGCGCGAAAACCC |
| | | | | GTCAGGGCGTTGAGGACGCGTTCTA |
| | | | | TACCCTGGTGCGTGAAATCCGTCAA |
| | | | | CACAAGCTGCGTAAACTGAACCCGC |
| | | | | CGGATGAAAGCGGTCCGGGCTGCAT |
| | | | | GAGCTGCAAGTGCGTTCTGAGCTAA |
| | | | | TGACTCGAG |
| GST-HRas- | 427 | MSPILGYWKIKGLVQPTRLLLEYLEE | 434 | GGATCCGACTACAAGGACGATGACG |
| G12V | | KYEEHLYERDEGDKWRNKKFELGLEF | | ATAAAATGACCGAGTATAAACTGGT |
| | | PNLPYYIDGDVKLTQSMAIIRYIADK | | GGTGGTGGGTGCGGTTGGCGTTGGT |
| | | HNMLGGCPKERAEISMLEGAVLDIRY | | AAAAGCGCGCTGACCATCCAGCTGA |
| | | GVSRIAYSKDFETLKVDFLSKLPEML | | TTCAAAACCACTTCGTGGACGAGTA |
| | | KMFEDRLCHKTYLNGDHVTHPDFMLY | | CGATCCGACCATCGAAGACAGCTAT |
| | | DALDVVLYMDPMCLDAFPKLVCFKKR | | CGTAAGCAGGTTGTGATCGATGGCG |
| | | IEAIPQIDKYLKSSKYIAWPLQGWQA | | AGACCTGCCTGCTGGACATTCTGGA |
| | | TFGGGDHPPKSDLVPRGSDYKDDDDK | | TACCGCGGGCCAGGAAGAGTACAGC |
| | | MTEYKLVVVGAVGVGKSALTIQLIQN | | GCGATGCGTGATCAATATATGCGTA |
| | | HFVDEYDPTIEDSYRKQVVIDGETCL | | CCGGTGAAGGCTTCCTGTGCGTTTT |
| | | LDILDTAGQEEYSAMRDQYMRTGEGF | | TGCGATTAACAACACCAAAAGCTTT |
| | | LCVFAINNTKSFEDIHQYREQIKRVK | | GAGGACATCCACCAGTACCGTGAAC |
| | | DSDDVPMVLVGNKCDLAARTVESRQA | | AAATTAAGCGTGTTAAAGATAGCGA |
| | | QDLARSYGIPYIETSAKTRQGVEDAF | | CGATGTGCCGATGGTTCTGGTGGGT |
| | | YTLVREIRQHKLRKLNPPDESGPGCM | | AACAAGTGCGACCTGGCGGCGCGTA |
| | | SCKCVLS | | CCGTGGAGAGCCGTCAGGCGCAAGA |
| | | | | TCTGGCGCGTAGCTACGGTATCCCG |
| | | | | TATATTGAAACCAGCGCGAAAACCC |
| | | | | GTCAGGGCGTTGAGGACGCGTTCTA |
| | | | | TACCCTGGTGCGTGAAATCCGTCAA |
| | | | | CACAAGCTGCGTAAACTGAACCCGC |
| | | | | CGGATGAAAGCGGTCCGGGCTGCAT |
| | | | | GAGCTGCAAGTGCGTTCTGAGCTAA |
| | | | | TGACTCGAG |
| GST-HRas- | 428 | MSPILGYWKIKGLVQPTRLLLEYLEE | 435 | GGATCCGACTACAAGGACGATGACG |
| G12D | | KYEEHLYERDEGDKWRNKKFELGLEF | | ATAAAATGACCGAGTATAAACTGGT |
| | | PNLPYYIDGDVKLTQSMAIIRYIADK | | GGTGGTGGGTGCGGATGGCGTTGGT |
| | | HNMLGGCPKERAEISMLEGAVLDIRY | | AAAAGCGCGCTGACCATCCAGCTGA |
| | | GVSRIAYSKDFETLKVDFLSKLPEML | | TTCAAAACCACTTCGTGGACGAGTA |
| | | KMFEDRLCHKTYLNGDHVTHPDFMLY | | CGATCCGACCATCGAAGACAGCTAT |
| | | DALDVVLYMDPMCLDAFPKLVCFKKR | | CGTAAGCAGGTTGTGATCGATGGCG |
| | | IEAIPQIDKYLKSSKYIAWPLQGWQA | | AGACCTGCCTGCTGGACATTCTGGA |
| | | TFGGGDHPPKSDLVPRGSDYKDDDDK | | TACCGCGGGCCAGGAAGAGTACAGC |
| | | MTEYKLVVVGADGVGKSALTIQLIQN | | GCGATGCGTGATCAATATATGCGTA |
| | | HFVDEYDPTIEDSYRKQVVIDGETCL | | CCGGTGAAGGCTTCCTGTGCGTTTT |
| | | LDILDTAGQEEYSAMRDQYMRTGEGF | | TGCGATTAACAACACCAAAAGCTTT |
| | | LCVFAINNTKSFEDIHQYREQIKRVK | | GAGGACATCCACCAGTACCGTGAAC |
| | | DSDDVPMVLVGNKCDLAARTVESRQA | | AAATTAAGCGTGTTAAAGATAGCGA |
| | | QDLARSYGIPYIETSAKTRQGVEDAF | | CGATGTGCCGATGGTTCTGGTGGGT |
| | | YTLVREIRQHKLRKLNPPDESGPGCM | | AACAAGTGCGACCTGGCGGCGCGTA |
| | | SCKCVLS | | CCGTGGAGAGCCGTCAGGCGCAAGA |
| | | | | TCTGGCGCGTAGCTACGGTATCCCG |
| | | | | TATATTGAAACCAGCGCGAAAACCC |
| | | | | GTCAGGGCGTTGAGGACGCGTTCTA |
| | | | | TACCCTGGTGCGTGAAATCCGTCAA |
| | | | | CACAAGCTGCGTAAACTGAACCCGC |
| | | | | CGGATGAAAGCGGTCCGGGCTGCAT |
| | | | | GAGCTGCAAGTGCGTTCTGAGCTAA |
| | | | | TGACTCGAG |
| VHH | 392 | MHHHHHHENLYFQGEVQLLESGGGLV | 402 | ATGCATCACCACCACCACCACGAGA |
| against | | QPGGSLRLSCAASGFTFSTFSMNWVR | | ACCTGTACTTCCAGGGCGAGGTGCA |
| HRas G12D | | QAPGKGLEWVSYISRTSKTIYYADSV | | ACTGCTGGAGAGCGGTGGCGGTCTG |
| with cys | | KGRFTISRDNSKNTLYLQMNSLRAED | | GTTCAACCGGGCGGTAGCCTGCGTC |
| | | TAVYYCARGRFFDYWGQGTLVTVSSC | | TGAGCTGCGCGGCGAGCGGTTTCAC |
| | | | | CTTTAGCACCTTTAGCATGAACTGG |
| | | | | GTGCGTCAAGCGCCGGGCAAGGGTC |
| | | | | TGGAGTGGGTTAGCTATATCAGCCG |
| | | | | TACCAGCAAGACCATTTACTATGCG |
| | | | | GACAGCGTGAAAGGCCGTTTCACCA |
| | | | | TCAGCCGTGATAACAGCAAAAACAC |
| | | | | CCTGTACCTGCAGATGAACAGCCTG |
| | | | | CGTGCGGAAGACACCGCGGTTTACT |
| | | | | ATTGCGCGCGTGGTCGTTTCTTTGA |
| | | | | TTATTGGGGCCAAGGTACCCTGGTG |
| | | | | ACCGTTAGCAGCTGCTAATGAAAGC |
| | | | | TT |
| VHH | 393 | MHHHHHHENLYFQGEVQLLESGGGLV | 403 | ATGCATCACCACCACCACCACGAGA |
| against | | QPGGSLRLSCAASGFTFSTFSMNWVR | | ACCTGTACTTCCAGGGCGAGGTGCA |
| HRAS | | QAPGKGLEWVSYISRTSKTIYYADSV | | ACTGCTGGAGAGCGGTGGCGGTCTG |
| G12D | | KGRFTISRDNSKNTLYLQMNSLRAED | | GTTCAACCGGGCGGTAGCCTGCGTC |
| | | TAVYYCARGRFFDYWGQGTLVTVSS | | TGAGCTGCGCGGCGAGCGGTTTCAC |
| | | | | CTTTAGCACCTTTAGCATGAACTGG |
| | | | | GTGCGTCAAGCGCCGGGCAAGGGTC |
| | | | | TGGAGTGGGTTAGCTATATCAGCCG |
| | | | | TACCAGCAAGACCATTTACTATGCG |
| | | | | GACAGCGTGAAAGGCCGTTTCACCA |
| | | | | TCAGCCGTGATAACAGCAAAAACAC |
| | | | | CCTGTACCTGCAGATGAACAGCCTG |
| | | | | CGTGCGGAAGACACCGCGGTTTACT |
| | | | | ATTGCGCGCGTGGTCGTTTCTTTGA |
| | | | | TTATTGGGGCCAAGGTACCCTGGTG |
| | | | | ACCGTTAGCAGCTAATGAAAGCTT |
| K19-cys | 394 | MHHHHHHENLYFQGDLGKKLLEAARA | 404 | ATGCATCACCACCACCACCACGAGA |
| | | GQDDEVRILMANGADVNASDRWGWTP | | ACCTGTACTTCCAGGGTGACCTGGG |
| | | LHLAAWWGHLEIVEVLLKRGADVSAA | | CAAGAAACTGCTGGAGGCGGCGCGT |
| | | DLHGQSPLHLAAMVGHLEIVEVLLKY | | GCGGGTCAAGACGATGAAGTGCGTA |
| | | GADVNAKDTMGATPLHLAARSGHLEI | | TCCTGATGGCGAACGGTGCGGACGT |
| | | VEELLKNGADMNAQDKFGKTTFDIST | | TAACGCGAGCGATCGTTGGGGCTGG |
| | | DNGNEDLAEILQKLC | | ACCCCGCTGCACCTGGCGGCGTGGT |
| | | | | GGGGTCACCTGGAGATTGTGGAAGT |
| | | | | TCTGCTGAAACGTGGTGCGGATGTG |
| | | | | AGCGCGGCGGATCTGCACGGCCAGA |
| | | | | GCCCGCTGCATCTGGCGGCGATGGT |
| | | | | GGGTCACCTGGAGATCGTGGAAGTT |
| | | | | CTGCTGAAGTATGGCGCGGATGTTA |
| | | | | ACGCGAAAGATACGATGGGTGCGAC |
| | | | | CCCGCTGCATTTAGCTGCGCGTAGC |
| | | | | GGCCACCTGGAAATTGTTGAGGAAC |
| | | | | TGCTGAAGAACGGTGCGGACATGAA |
| | | | | CGCGCAGGATAAGTTCGGCAAAACC |
| | | | | ACCTTTGACATCAGCACCGATAACG |
| | | | | GTAACGAGGATCTGGCGGAAATTCT |
| | | | | GCAAAAACTGTGCTAATGAAAGCTT |
| K19 | 395 | MHHHHHHENLYFQGDLGKKLLEAARA | 405 | ATGCATCACCACCACCACCACGAGA |
| | | GQDDEVRILMANGADVNASDRWGWTP | | ACCTGTACTTCCAGGGTGACCTGGG |
| | | LHLAAWWGHLEIVEVLLKRGADVSAA | | CAAGAAACTGCTGGAGGCGGCGCGT |
| | | DLHGQSPLHLAAMVGHLEIVEVLLKY | | GCGGGTCAAGACGATGAAGTGCGTA |
| | | GADVNAKDTMGATPLHLAARSGHLEI | | TCCTGATGGCGAACGGTGCGGACGT |
| | | VEELLKNGADMNAQDKFGKTTFDIST | | TAACGCGAGCGATCGTTGGGGCTGG |
| | | DNGNEDLAEILQKL | | ACCCCGCTGCACCTGGCGGCGTGGT |
| | | | | GGGGTCACCTGGAGATTGTGGAAGT |
| | | | | TCTGCTGAAACGTGGTGCGGATGTG |
| | | | | AGCGCGGCGGATCTGCACGGCCAGA |
| | | | | GCCCGCTGCATCTGGCGGCGATGGT |
| | | | | GGGTCACCTGGAGATCGTGGAAGTT |
| | | | | CTGCTGAAGTATGGCGCGGATGTTA |
| | | | | ACGCGAAAGATACGATGGGTGCGAC |
| | | | | CCCGCTGCATTTAGCTGCGCGTAGC |
| | | | | GGCCACCTGGAAATTGTTGAGGAAC |
| | | | | TGCTGAAGAACGGTGCGGACATGAA |
| | | | | CGCGCAGGATAAGTTCGGCAAAACC |
| | | | | ACCTTTGACATCAGCACCGATAACG |
| | | | | GTAACGAGGATCTGGCGGAAATTCT |
| | | | | GCAAAAACTGTAATGAAAGCTT |

### Production of K19 Containing Degradation Constructs

A series of degradation constructs that included K19 and a truncated natural E3 ubiquitin ligase or a E3 ubiquitin ligase recruiter were designed and produced. Table 22 shows the degradation constructs used.

In addition to fusion with E3 ligases and active fragments of E3 ligases to induce target degradation, K19 was also fused with human IgG1-Fc as well as with minibody (Mb). Particularly, K19 was fused with human IgG1-Fc(N297A). The Mb was generated by fusing hinge plus CH3 domain derived from human IgG1-Fc. CH3 domains in Mb and Fc fusions were expected to engage with TRIM21 for target degradation. Additional cysteine at N or C-terminus, or S239C mutation on Fe were used for EEV conjugation.

TABLE 22
Degradation construct design

| | Protein | | DNA |
|---|---|---|---|
| | SEQ | | SEQ |
| K19 degradation | ID | | ID |
| constructs | NO: | Mutations or additional amino acids | NO: |
| K19-Fc-Cys | | Fc (N297A), C-terminal Cys | |
| FCS_{239C}-K19 | | Fc (N297A, S239C) | |
| K19-Mb-Cys | | C-terminal Cys | |
| Cys-K19-Mb- | | N-terminal Cys, C-terminal VHL₁₅₂₋₂₁₃ | |
| VHL₁₅₂₋₂₁₃ | | | |
| VHL₁₅₂₋₂₁₃-K19- | | C-terminal Cys, N-terminal VHL₁₅₂₋₂₁₃ | |
| Mb-Cys | | | |
| Cys-K19- | | N-terminal Cys, C-terminal TRIM21₁₋₂₇₇ | |
| TRIM21₁₋₂₇₇ | | | |
| TRIM21₁₋₂₇₇- | | C-terminal Cys, N-terminal TRIM21₁₋₂₇₇ | |
| K19-Cys | | | |
| Cys-7D12-K19 | | N-terminal Cys, bispecific anti-EGFR | |
| | | (7D12) VHH & K19 | |
| 7D12-K19-Cys | | C-terminal Cys, bispecific anti-EGFR | |
| | | (7D12) VHH and K19 | |
| Cys-7D12-K19- | | N-terminal Cys, C-terminal VHL₁₅₂₋₂₁₃, | |
| VHL₁₅₂₋₂₁₃ | | bispecific anti-EGFR (7D12) | |
| | | VHH and K19 | |
| Cys-7D12-K19- | | N-terminal Cys, C-terminal VHLpep, | |
| VHLpep2a | | bispecific anti-EGFR (7D12) | |
| | | VHH and K19 | |
| Cys-7D12-K19- | | N-terminal Cys, C-terminal TRIM21₁₋₂₇₇, | |
| TRIM21₁₋₂₇₇ | | bispecific anti-EGFR (7D12) | |
| | | VHH and K19 | |
| Cys-K19-7D12- | | | |
| SOCS1pep | | | |
| VHL₁₋₁₂₃-K19 | | | |
| VHL₁₅₇₋₁₉₄-K19 | | | |

The P. pastoris expression vector (P1) was used for the expression of degradation constructs in Table T13. The vector consists of Pichia alcohol oxidase 1 (AOX1) promoter to drive recombinant protein expression as an inducible promotor and the AOX1 transcriptional terminator (TT). Antibiotic selection marker is Shble (Zeocin®) and the plasmid carries a Pichia TRP2 for a genomic integrating locus. The gene of interest was codon optimized for P. pastoris and cloned into the P1 at EcoRI and FseJ sites. The recombinant plasmid is digested with SpeI and transformed into wild-type Pichia pastoris (NRRL Y-11430) by electroporation according to a manufacture's manual. The resulting transformants that are resistant to Zeocin were screened using a 96 deep-well plate.

The recombinant plasmid is digested with SpeI and transformed into wild-type Pichia pastoris (NRRL Y-11430) by electroporation according to a manufacture's manual. The resulting transformants that are resistant to Zeocin were screened using a 96 deep-well plate.

For small cultures for recombinant protein expression, individual transformants were inoculated into individual wells containing 600 μL buffered glycerol-complex medium (BMGY) consisting of 1% yeast extract, 2% peptone, 100 mM potassium phosphate buffer (pH 6.0), 1.34% yeast nitrogen base, 0.4 ug/mL biotin, and 1% glycerol in a 96 deep-well plate. The cultures were allowed to grow at 30° C. and 350 RPM. After 48 hours, BMGY is replaced with 600 μL BMMY (buffered methanol-complex medium, pH6.0: same as BMGY except 1% methanol instead of 1% glycerol), and recombinant protein is induced for 48 h. Methanol is added to each well every 24 h up to 1%. Production strains were selected based SDS-PAGE analysis.

For medium cultures, transformants from the 96 deep-well plate screening were inoculated into 10 mL BMGY to prepare a seed culture. The seed culture was then inoculated into 1 L BMGY in a 5 L baffled shake flask and grown to an OD₆₀₀ of 2-6 while incubating at 30 and 250 RPM. The culture was centrifuged, and the cell pellet was resuspended in 100 mL BMMY. Recombinant protein was induced for 48 h. Methanol was added every 24 h up to 1%. The secreted protein was harvested by pelleting the biomass by centrifugation and the supernatant was collected for purification.

For larger scale protein production, fed-batch fermentation was carried out in a 3 L bioreactor with the initial working volume of 1.17 L. A single colony was inoculated into 50 mL YPD medium and cultivated to reach 2-6 OD₆₀₀ for 24 h at 30° C. and 250 rpm. The seed culture was inoculated into a bioreactor containing basal media (pH6.5): 1.17 L PBM1 (10 g/L Yeast Extract, 20 g/L Soytone, 13.4 g/L YNB with Ammonium Sulfate, 5.6 g/L Dibasic Potassium Phosphate, 8.6 g/L Monobasic Potassium Phosphate, 40 g/L Glycerol, 18.6 g/L Sorbitol, 8 mg/L Biotin, 75 mg/L Antifoam 204. Batch mode was run until all the glycerol was exhausted as indicated by sharp DO spike and then fed-batch with 50% (w/w) glycerol feed containing 0.75 mg biotin and 1.875 PTM2 was started at the constant feed rate of 18.15 mL/L/h) for 8 h. After the glycerol fed-batch, methanol fed-batch was started with 100% (v/v) methanol feed containing 2 mg Biotin, 5 mL PTM2) at the feed rate of 5.45 mL/L/h for 48 h. The pH and DO level were maintained throughout the fermentation at 6.5 and above 30% by adjusting agitation rate (450 rpm to 100 rpm), respectively. The degradation constructions were lysed and purified from the culture supernatant using HITRAP MABSELECT, size exclusion chromatography, or AMSPHERE A33.

Method A using HiTrap MabSelect PrismA column: Harvested Pichia media is centrifuged at 3000×g for 10 min to clarify and remove remaining cellular debris and transferred to a falcon tube. The column is then equilibrated with 5 CV of equilibration buffer (20 mM Sodium Phosphate+150 mM NaCl, pH 7.4). The sample is then loaded onto a pre-packed 5 mL HiTrap MabSelect PrismA column at 2.0 ml/min. After sample is loaded, 5 CV of equilibration buffer is run over the column followed by 5 CV of Wash Buffer 1 (Wash 1: 20 mM Sodium Phosphate+500 mM NaCl, pH 7.4) and 5 CV of Wash Buffer 2 (50 mM Sodium Acetate, pH 6). Protein is eluted off the column with 5 CVs of elution buffer (Elution: 50 mM Sodium Acetate, pH 3.0). Column is regenerated with 15-minute contact time of 0.1M NaOH, 5 CV H₂O, and 5 CV of equilibration buffer (50 mM Sodium Acetate, pH 3.5).

Method B using size exclusion purification: Pichia Supernatant is four times concentrated (8 mL to 2 mL using Amicon Ultra-15 Centrifugal Filters; 10 KDa MW Cutoff) before injecting onto a 100 mL Sephacryl S-200 High Resolution size exclusion chromatography column at 2 mL/min. Phosphate buffered saline (PBS) is used as running buffer and fractions containing non-aggregate protein of interest are collected and pooled. Elutions are then further concentrated to 1 mg/mL using Amicon Ultra-15 Centrifugal Filters (10 KDa MW Cutoff).

Method C using Amsphere A3 column: Harvested Pichia media is centrifuged at 3000×g for 10 min to clarify and remove remaining cellular debris and transferred to a falcon tube. The column (Nick: do you know the catalogue and vendor for this column?) is then equilibrated with 5 CV of equilibration buffer (20 mM Sodium Phosphate, pH 7.5). The sample is then loaded onto a 5 mL column at 2.0 ml/min. After sample is loaded, 5 CV of equilibration buffer is run over the column followed by 5 CV of wash buffer (20 mM Sodium Phosphate+0.5M NaCl, pH 7.5). The column is then equilibrated in an additional 5 CV of equilibration buffer. Protein is eluted off the column with 5 CVs of elution buffer (Elution: 50 mM Sodium Acetate, pH 3.0). Column is regenerated with 15-minute contact time of 0.1M NaOH, 5 CV H2O, and 5 CV of equilibration buffer.

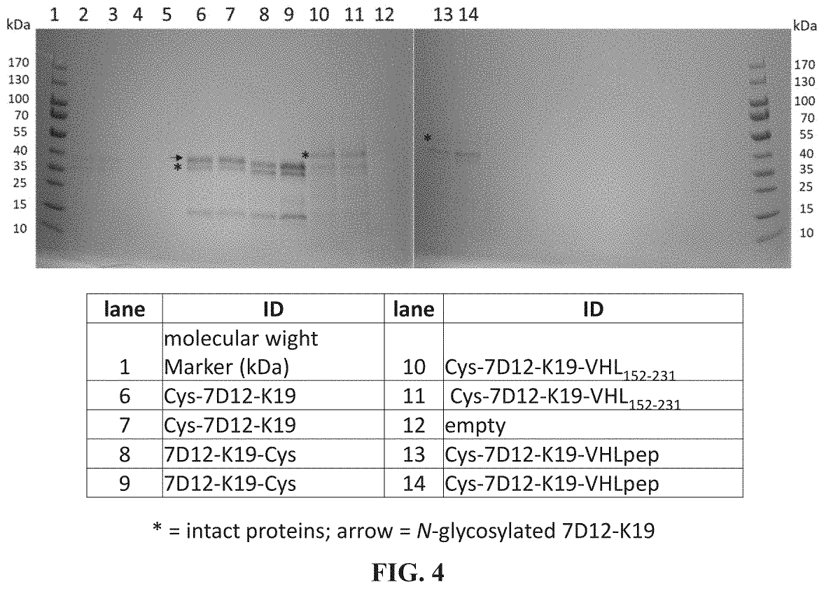

FIG. 4 is an SDS-PAGE gel showing the purity of various constructs. The desired molecular weight was visible for the constructs (asterisk). The lower molecular weight bands are degraded proteins. Additionally, N-glycosylated 7D12-K19 was observed (arrow).

### Example 2: Bioconjugation of K19 with and EEV

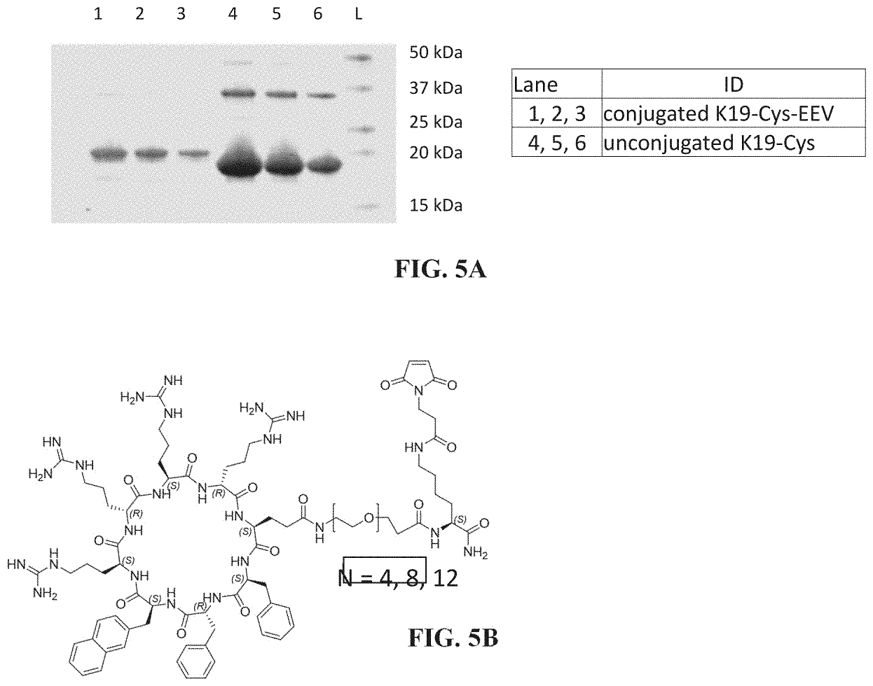

K19-Cys was conjugated to an EEV12 (cyclo(FfΦRrRrQ), more specifically cyclo(FfΦRrRrQ)-PEG-K(mal); FIG. 5B); PEG=PEG 8 or PEG 12) while the K19-cys (with his tag) while the protein was still immobilized on the resin. The His-trap resin was suspended in PBS, 500 mM NaCl, 20% glycerol, 1.1 eq. EEV12-PEG₈-maleimide or EEV12-PEG₁₂-maleimide and reacted overnight at room temperature with gentle mixing. Extent of conjugation was determined by SDS-PAGE. Once the conjugation was complete, the conjugated protein was eluted with PBS, 500 mM NaCl, 250 mM imidazole, 20% glycerol, pH 7.4. The conjugated protein was buffer exchanged into PBS, 20% glycerol, pH 7.4, aliquoted and snap frozen. The extent of conjugation for various batches was analyzed by SDS-PAGE (FIG. 5A; each lane is a batch). The increase in molecular weight from the unconjugated to the conjugated protein suggests successful conjugation of EEV12 to K19-cys.

### Biophysical Characterizations Unconjugated and Conjugated K19

Binding affinity of K19-Cys and K19-Cys-EEV12 to GST-KRas(1-188)-GDP or GST-KRas^{G12D}(1-188)-GDP was determined by biolayer interferometry. Ni-NTA biosensors (ForteBio) were first equilibrated in 10×KB Buffer for 10 min. before being dipped sequentially into: 10×KB buffer for 60 s for baseline; 250 nM K19-Cys or 250 nM K19-Cys-EEV12 in 10×KB buffer, 150 s. for biosensor loading; 10× KB buffer, 60 s for post-loading baseline; 625 nM, 125 nM, 25 nM GST-KRas(1-188)-GDP and GST-KRas^{G12D}(1-188)-GDP in 10×KB buffer, 120 s, for association; 10×KB buffer, 20 min, for dissociation. Sensorgrams were processed using Octet Data Analysis HT (ForteBio) and a 1:1 binding mode to determine kₐ, k_{d}, and K_{D}. K19 and its EEV conjugate (K19-Cys-EEV12) can bind with GST-KRas^{G12D}(1-188)-GDP with dissociation constant (K_{D}) at 6.82 nM (kₐ=2.16×10⁴ M⁻¹ s⁻¹; k_{d}=1.48×10⁻⁴ s⁻¹) and 3.15 nM (kₐ=2.63×10⁴ M⁻¹ s⁻¹; k_{d}=7.39×10⁻⁵ s⁻¹) respectively.

### In Vitro Pharmacological Effects of K19 and K19-EEV12 on Cancer Cell Lines

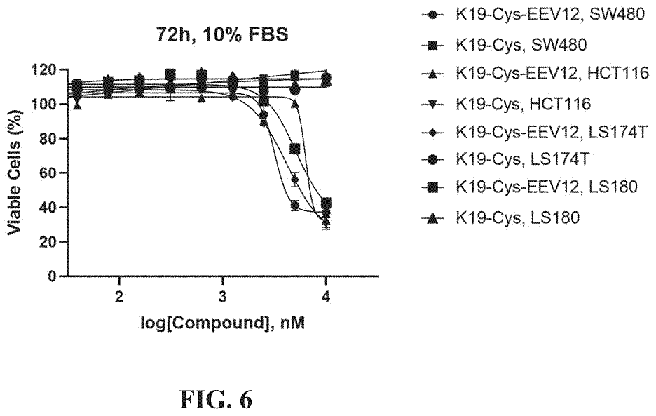

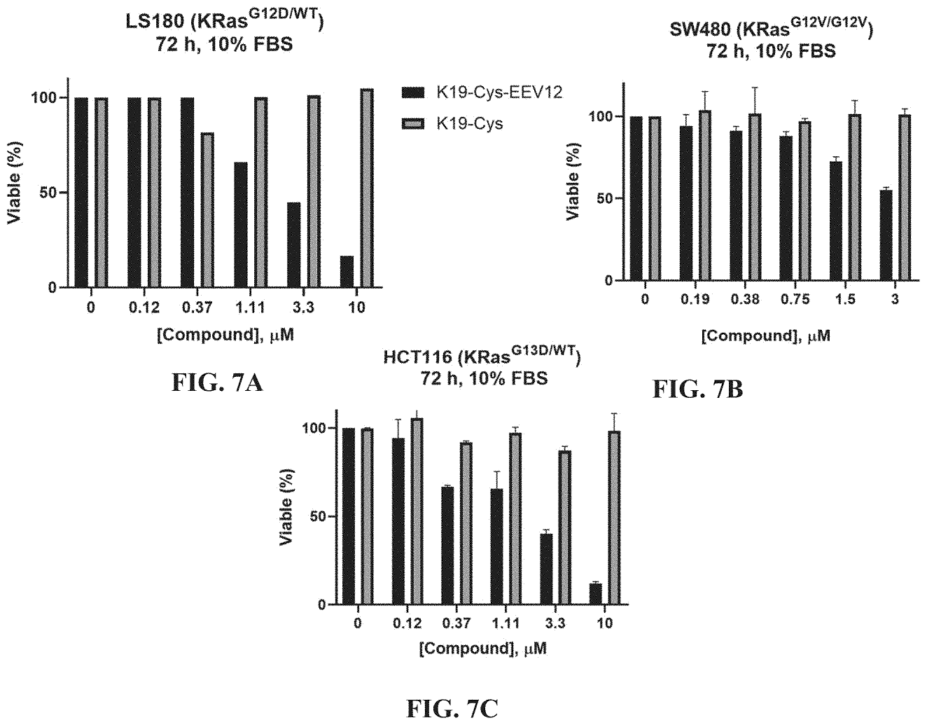

Delivery of K19 against KRas protein was investigated in vitro. Effects of K19-Cys and K19-Cys-EEV12 on cell viability were determined by seeding 2.5×10³ HCT-116, SW480, LS180, or LS174T cells in clear tissue culture-treated 96-well plates in complete growth media (McCoy's 5A or RPMI-1640) and incubated O/N at 37° C., 5% CO₂. Compounds were serially diluted in sterile water and added to cells before incubation for 72 h at 37° C., 5% CO₂. After incubation, 150 μL of CELLTITER-GLO 2.0 reagent was added to each well and shaken for 30 min. at RT. 200 μL from each well was transferred to opaque 96-well plates before measuring luminescence using a SPECTRAMAX ID5 (Molecular Devices) using the manufacturer's recommended settings. Cell viability was determined relative to vehicle-treated wells and plotted using GraphPad Prism v.8 (FIG. 6). Conjugated K19-Cys-EEV12 showed dose-dependent anti-proliferative activities on multiple KRas mutant cell lines, with IC₅₀ values ranging from 2-8 μM, while the unconjugated K19-Cys showed minimal activities up to 10 μM (FIG. 7A-C).

### Pharmacological Effects of K19 and K19-EEV12 on KRas Signaling Pathways in Cancer Cell Lines

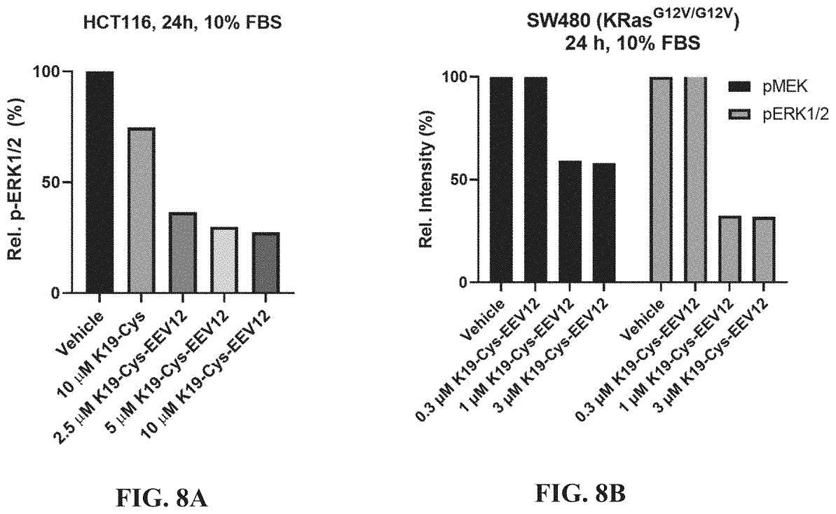

Effects of K19-Cys and K19-Cys-EEV12 on the Ras signaling pathway on human cancer cell lines with KRas mutations were determined by western blotting. 1×10⁶ HCT-116 or SW480 cells were seeded in complete growth media (McCoy's 5A or RPMI-1640 respectively) in 6-well tissue culture-treated plates and incubated O/N at 37° C., 5% CO₂. The next day, compounds were diluted in sterile water and added to wells to the indicated final concentration and incubated for 24 h. Adherent and non-adherent cells were collected by scraping and centrifuging the growth media (1000 g, 5 min., 4° C.), respectively, combined, and centrifuged again (1000 g, 5 min., 4° C.) to pellet the cells. The supernatant was discarded, and the cells were lysed in 1×RIPA buffer supplemented with complete Protease Inhibitor Cocktail, EDTA-Free and Phosphatase Inhibitor Cocktails 1, 2, and 3; and homogenized by passing through a syringe. Conjugated K19-Cys-EEV12 demonstrated potent reduction in KRas downstream effector phosphorylation in mutant KRas cell lines HCT116 (FIG. 8A) and SW480 (FIG. 8B) after treatment for 24 h, while unconjugated K19-Cys had negligible activity.

### Example 3: Evaluation of Various β-Catenin Targeting Constructs

Various BC2 degradation constructs where designed, produced, and evaluated for targeting β-catenin.

Human β-catenin including a N-terminal His tag and a TEV protease cleavage site was recombinantly expressed in BL21 star (DE3) E. Coli. Briefly, a culture was inoculated in TB medium with the selectable maker kanamycin and cultured at 37° C. while shaking until the OD₆₀₀ reached 1.2. Protein expression was induced with IPTG at 15° C. for 16 hours. Cells were lysed and purified via Ni affinity chromatography. Protein purity and molecular weight were determined by standard SDS-PAGE along with Western blot confirmation (mouse anti-His-mAb).

Human β-catenin
(SEQ ID NO: 436)
MGSSHHHHHHSSGENLYFQGHMASMTGGEEMGRGSMATQADLMELDMAME
PDRKAAVSHWQQQSYLDSGIHSGATTTAPSLSGKGNPEEEDVDTSQVLYE
WEQGFSQSFTQEQVADIDGQYAMTRAQRVRAAMFPETLDEGMQIPSTQFD
AAHPTNVORLAEPSQMLKHAVVNLINYQDDAELATRAIPELTKLLNDEDQ
VVVNKAAVMVHQLSKKEASRHAIMRSPOMVSAIVRTMQNTNDVETARCTA
GTLHNLSHHREGLLAIFKSGGIPALVKMLGSPVDSVLFYAITTLHNLLLH
QEGAKMAVRLAGGLQKMVALLNKTNVKFLAITTDCLQILAYGNQESKLII
LASGGPQALVNIMRTYTYEKLLWTTSRVLKVLSVCSSNKPAIVEAGGMQA
LGLHLTDPSQRLVQNCLWTLRNLSDAATKQEGMEGLLGTLVQLLGSDDIN
VVTCAAGILSNLTCNNYKNKMMVCQVGGIEALVRTVLRAGDREDITEPAI
CALRHLTSRHQEAEMAQNAVRLHYGLPVVVKLLHPPSHWPLIKATVGLIR
NLALCPANHAPLREQGAIPRLVQLLVRAHQDTQRRTSMGGTQQQFVEGVR
MEEIVEGCTGALHILARDVHNRIVIRGLNTIPLFVQLLYSPIENIQRVAA
GVLCELAQDKEAAEAIEAEGATAPLTELLHSRNEGVATYAAAVLFRMSED
KPQDYKKRLSVELTSSLFRTEPMAWNETADLGLDIGAQGEPLGYRQDDPS
YRSFHSGGYGQDALGMDPMMEHEMGGHHPGADYPVDGLPDLGHAQDLMDG
LPPGDSNQLAWFDTDL
Human β-catenin
(SEQ ID NO: 437)
ATGGCTACACAAGCGGACCTGATGGAGCTGGATATGGCGATGGAGCCGGA
TCGTAAGGCGGCGGTTAGCCACTGGCAGCAACAGAGCTACCTGGATAGCG
GCATCCACAGCGGTGCGACCACCACCGCGCCGAGCCTGAGCGGTAAAGGC
AACCCGGAGGAAGAGGACGTTGATACCAGCCAGGTGCTGTACGAATGGGA
GCAGGGCTTCAGCCAGAGCTTCACCCAGGAGCAAGTTGCGGACATCGATG
GCCAGTATGCGATGACCCGTGCGCAACGTGTGCGTGCGGCGATGTTCCCG
GAAACCCTGGATGAGGGTATGCAGATTCCGAGCACCCAATTTGACGCGGC
GCACCCGACCAACGTTCAGCGTCTGGCGGAACCGAGCCAAATGCTGAAGC
ACGCGGTGGTTAACCTGATCAACTATCAGGACGATGCGGAACTGGCGACC
CGTGCGATTCCGGAGCTGACCAAGCTGCTGAACGACGAAGATCAGGTGGT
TGTGAACAAAGCGGCGGTGATGGTTCACCAACTGAGCAAGAAAGAGGCGA
GCCGTCACGCGATCATGCGTAGCCCGCAGATGGTTAGCGCGATTGTGCGT
ACCATGCAAAACACCAACGATGTGGAAACCGCGCGTTGCACCGCGGGTAC
CCTGCACAACCTGAGCCACCACCGTGAAGGTCTGCTGGCGATCTTCAAGA
GCGGTGGCATTCCGGCGCTGGTTAAAATGCTGGGCAGCCCGGTGGACAGC
GTTCTGTTTTACGCGATCACCACCCTGCACAACCTGCTGCTGCACCAGGA
AGGTGCGAAAATGGCGGTGCGTCTGGCGGGTGGCCTGCAAAAAATGGTTG
CGCTGCTGAACAAGACCAACGTGAAATTCCTGGCGATCACCACCGACTGC
CTGCAGATTCTGGCGTATGGCAACCAAGAGAGCAAGCTGATCATTCTGGC
GAGCGGTGGCCCGCAGGCGCTGGTTAACATCATGCGTACCTACACCTATG
AAAAACTGCTGTGGACCACCAGCCGTGTGCTGAAGGTTCTGAGCGTGTGC
AGCAGCAACAAACCGGCGATTGTTGAGGCGGGTGGCATGCAAGCGCTGGG
TCTGCACCTGACCGATCCGAGCCAGCGTCTGGTGCAAAACTGCCTGTGGA
CCCTGCGTAACCTGAGCGACGCGGCGACCAAACAGGAAGGTATGGAGGGC
CTGCTGGGTACCCTGGTGCAACTGCTGGGCAGCGACGATATCAACGTTGT
GACCTGCGCGGCGGGTATTCTGAGCAACCTGACCTGCAACAACTACAAGA
ACAAAATGATGGTTTGCCAAGTGGGTGGCATCGAGGCGCTGGTTCGTACC
GTGCTGCGTGCGGGCGACCGTGAAGATATCACCGAGCCGGCGATTTGCGC
GCTGCGTCACCTGACCAGCCGTCACCAGGAAGCGGAGATGGCGCAAAACG
CGGTTCGTCTGCACTATGGTCTGCCGGTTGTGGTTAAGCTGCTGCACCCG
CCGAGCCACTGGCCGCTGATCAAAGCGACCGTGGGCCTGATTCGTAACCT
GGCGCTGTGCCCGGCGAACCATGCGCCGCTGCGTGAACAGGGTGCGATTC
CGCGTCTGGTTCAACTGCTGGTGCGTGCGCACCAGGATACCCAACGTCGT
ACCAGCATGGGTGGCACCCAACAGCAATTCGTTGAGGGTGTGCGTATGGA
AGAGATCGTTGAAGGCTGCACCGGTGCGCTGCACATTCTGGCGCGTGACG
TTCACAACCGTATCGTGATTCGTGGCCTGAACACCATCCCGCTGTTTGTG
CAGCTGCTGTACAGCCCGATCGAGAACATTCAACGTGTTGCGGCGGGTGT
GCTGTGCGAACTGGCGCAGGATAAGGAAGCGGCGGAGGCGATTGAGGCGG
AGGGTGCGACCGCGCCGCTGACCGAACTGCTGCACAGCCGTAACGAGGGT
GTTGCGACCTACGCGGCGGCGGTGCTGTTCCGTATGAGCGAGGACAAACC
GCAGGATTATAAGAAACGTCTGAGCGTTGAACTGACCAGCAGCCTGTTTC
GTACCGAACCGATGGCGTGGAACGAAACCGCGGACCTGGGCCTGGATATT
GGCGCGCAGGGTGAACCGCTGGGTTACCGTCAAGACGATCCGAGCTATCG
TAGCTTTCATAGCGGTGGCTACGGTCAGGATGCGCTGGGTATGGACCCGA
TGATGGAACATGAGATGGGTGGCCATCACCCGGGTGCGGACTATCCGGTG
GATGGCCTGCCGGACCTGGGTCACGCGCAAGACCTGATGGATGGCCTGCC
GCCGGGCGATAGCAATCAACTGGCGTGGTTTGACACCGACCTGTAATGAG
AATT

BC2, a β-catenin sdAb, discovered by screening a phage library generated from llama immunization with β-catenin antigen was fused to various E3 ligases and active fragments of E3 ligases. The degradation constructs are shown in Table 23.

TABLE 23
Degradation construct design

| BC2- | Protein | DNA | |
|---|---|---|---|
| specific fusion | SEQ ID | SEQ ID | Mutations or |
| proteins | NO: | NO: | additional amino acids |
| BC2-Cys | | | BC2 nanobody (VHH) |
| BC2-Fc-Cys | | | Fc (N297A), C-terminal Cys |
| BC2-Fc_{S239C} | | | Fc (N297A, S239C) |
| BC2-Mb-Cys | | | C-terminal Cys |
| Cys-BC2-Mb- | | | N-terminal Cys, C-terminal |
| VHL₁₅₂₋₂₁₃ | | | VHL₁₅₂₋₂₁₃ |
| VHL₁₅₂₋₂₁₃-BC2-Mb- | | | C-terminal Cys , N-terminal |
| Cys | | | VHL₁₅₂₋₂₁₃ |
| Cys-BC2-VHLpep | | | N-terminal Cys, C-terminal |
| | | | VHL peptide |
| VHLpep-BC2-Cys | | | C-terminal Cys, N-terminal |
| | | | VHL peptide |
| Cys-BC2_{VHH}- | | | N-terminal Cys, C-terminal |
| SOCS1pep | | | SOCS1 peptide |
| SOCS1pep-BC2-Cys | | | C-terminal Cys, N-terminal |
| | | | SOCS1 peptide |
| Cys-VHL₁₅₂₋₂₁₃- | | | N-terminal VHL & C-terminal |
| BC2-SOCS1pep | | | SOCS1pep |
| VHL_{152-213 (Y185F)}- | | | N-terminal VHL₁₅₂₋₂₁₃ (Y185F) |
| BC2-Cys | | | |
| Cys-BC2- | | | C-terminal TRIM21₁₋₂₇₇ |
| TRIM21₁₋₂₇₇ | | | |
| TRIM21₁₋₂₇₇-BC2- | | | N-terminal TRIM21₁₋₂₇₇ |
| Cys | | | |
| VHL₁₅₂₋₂₁₃-BC2- | | | N-terminal VHL & C-terminal |
| TRIM21₁₋₂₇₇-Cys | | | TRIM21₁₋₂₇₇ |
| BC2-7D12-cys | | — | — |

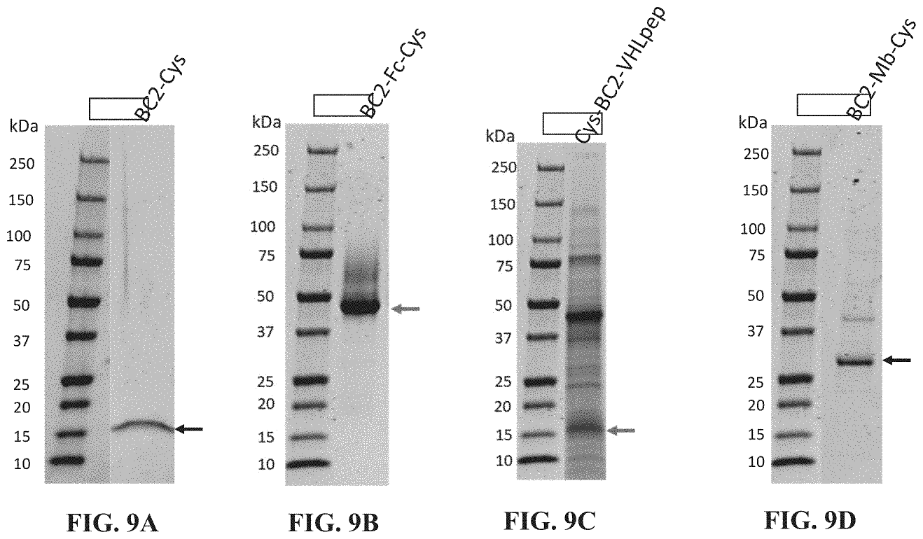

The P. pastoris expression vector (P1) was used for the expression of construct in Table 23 using methods similar to Example 1 for the small, medium, and large cultures. Constructs were purified from the culture using HITRAP, MABSELECT, or AMSPHERE A3 (using similar methods as described herein above). Constructs were evaluated using SDS-PAGE (examples are shown in FIG. 9A-D).

### Example 4: Bioconjugation of BC2 Constructs with EEV12

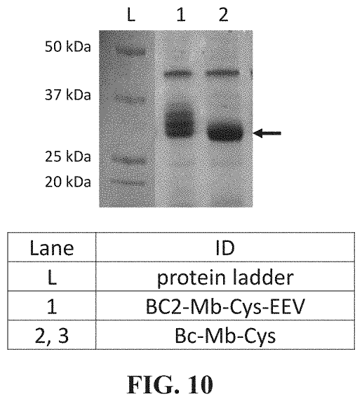

BC2-cys, BC2-Fc-cys and BC2-Mb-cys from Example 3 were conjugated to EEV12 (see Example 2 for sequence and structure). Conjugation was performed in PBS, 10% glycerol, pH 7.4 by first pre-incubating the protein with 1 mM TCEP for 1 h at RT with gentle mixing. After 1 h, the protein was desalted and 1.1 eq. of EEV12-PEG₁₂-malemide was added and allowed to react overnight at RT with gentle mixing. The extent of conjugation was determined by SDS-PAGE and the conjugated protein was desalted and exchanged into PBS, 10% glycerol, pH 7.4, aliquoted, and snap-frozen. The extent of conjugation was analyzed by unreduced SDS-PAGE analysis (example show in FIG. 10). The increase in molecular weight from unconjugated BC2-Mb-Cys and BC2-Mb-Cys-EEV12 indicates the conjugation reaction proceeded.

### Biophysical Characterizations of BC2 and BC2-EEV12 Constructs

Binding affinity of BC2-Cys, BC2-Cys-EEV12, BC2-Fc-Cys, BC2-Fc-Cys-EEV12, BC2-Mb-Cys, and BC2-Mb-Cys-EEV12 (from Example 3 and 4) to full-length human β-catenin (from Example 3) was determined by biolayer interferometry using methods similar to Example 3. A 1:1 binding mode was used to determine kₐ, k_{d}, and K_{D} for BC2-Cys (K_{D}=˜40 nM) and BC2-Cys. A 2:1 binding mode to determine kₐ, k_{d}, and K_{D} was used for BC2-Fc-Cys (K_{D}=<1 nM), BC2-Fc-Cys-EEV12 (K_{D}=>1 nM), BC2-Mb-Cys (K_{D}=˜0.6 nM), and BC2-Mb-Cys-EEV12 (K_{D}=˜1 nM). BC2-cys showed a K_{D} of 40 nM. The literature K_{D} for BC2 is 3.1 nM (Traenkle et al., Mol. Cell Proteomics (2015), 3: 707-723). The minibody and Fc fusion BC2s showed enhanced affinity to the target proteins with dissociation constants lower than 1 nM. The EEV conjugation through C-terminal Cys do not interfere with β-catenin binding as indicated by the biolayer interferometry analysis.

### Pharmacological Effects of BC2 and BC2-EEV12 Constructs in Cancer Cell Lines

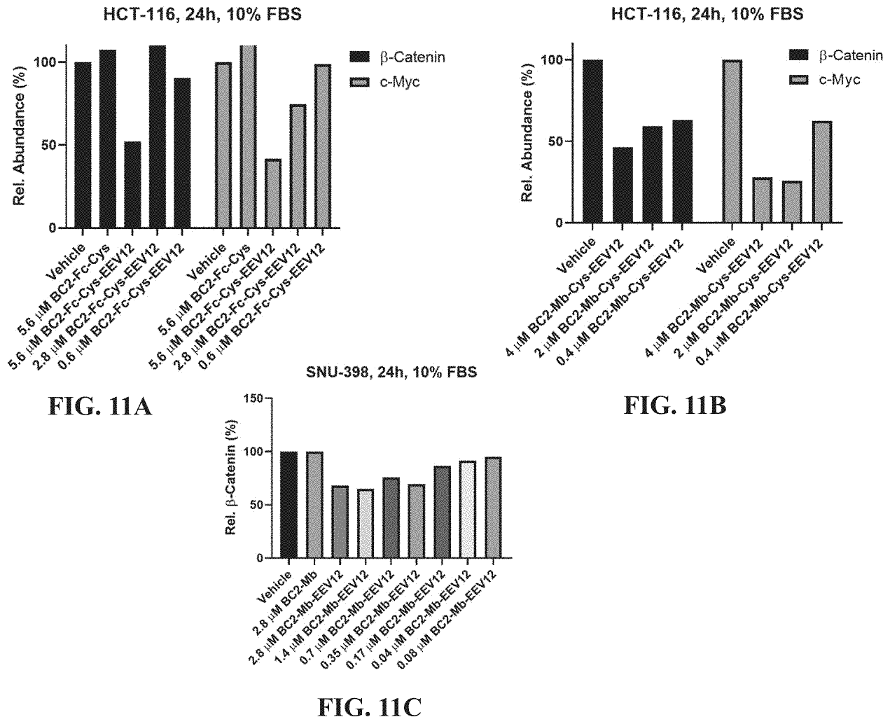

Effects of BC2-Cys, BC2-Cys-EEV12, BC2-Fc-Cys, BC2-Fc-Cys-EEV12, BC2-Mb-Cys, and BC2-Mb-Cys-EEV12 (from Example 3 and 4) on the Wnt/β-catenin signaling pathway in HCT-116 and SNU-398 were determined by western blotting. Methods similar to Example 2 were used. BC2-Fc-Cys-EEV12 but not unconjugated BC2-Fc-Cys demonstrated marked reductions in both β-catenin and c-Myc levels after 24 h treatment in HCT-116 (FIG. 11A). Similarly, BC2-Mb-Cys-EEV12 but not BC2-Mb-Cys demonstrated marked reductions in both β-catenin and c-Myc levels after 24 h treatment in HCT-116 cells (FIG. 11B) and SNU-398 cells (FIG. 11C).

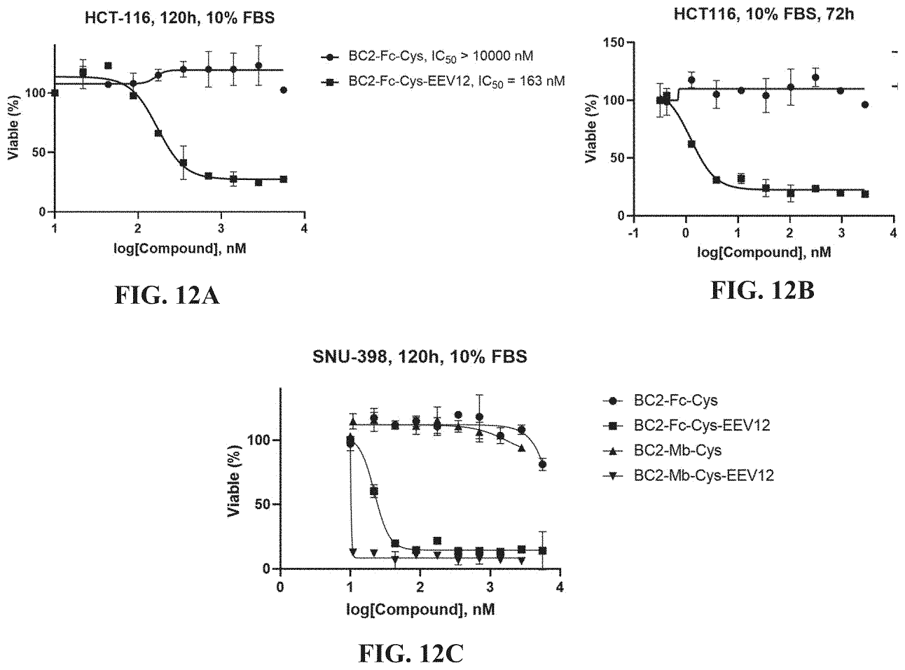

Anti-proliferative effects of BC2-Fc-Cys, BC2-Fc-Cys-EEV12, BC2-Mb-Cys, and BC2-Mb-Cys-EEV12 (from Example 3 and 4) on cell viability of HCT-116 and SNU-398 cells were determined similar to methods described in Example 2. Both BC2-Fc-Cys-EEV12 and BC2-Mb-Cys-EEV12 showed dose-dependent reduction of cell viability with IC50 of 163 nM and 1.2 nM on HCT116 cancer cell line (FIGS. 12A and 12B). In contrast, unconjugated BC2-Fc-Cys and BC2-Mb-Cys don't shown significantly antiproliferative activity (FIGS. 12A and 12B). For the SNU398 cell line, treatment with BC2-Fc-Cys-EEV12 and BC2-Mb-Cys-EEV12 reduced the viability with IC50 of 22 nM and <1 nM respectively (FIG. 12C).

### Example 5: Methods for Generating sdAb Against β-Catenin Proteins

Generation of VHH sdAb Using Immune Library and Phage Display

VHH immunized library will be generated by the llama immunization. In brief, naïve llama was immunized with purified β-catenin antigen using a standard protocol concerning antigen, adjuvant, immunogenicity and blood collection. Blood titer against immunogen was evaluated in ELISA experiment after 3^{rd} immunization. The peripheral blood mononuclear cell samples (PBMC) was collected from the llama and then the total RNA was isolated. The VHH gens were amplified with specific primers and inserted into the M13 phage display vector or Pichia display vector for construction of the phage display library. The quality of the immunized β-catenin VHH library was assessed by 96 randomly picked colonies. The library had an estimated size of 9.8×10⁸, in-frame rate of 94%, and the length of IGHV-CDR3 region ranges from 8 amino acids to 27 amino acids.

Phage display selection experiment was performed to screen β-catenin specific VHH. In brief, purified β-catenin protein was coated to an incubation well or tube through either direct coating or a capture system. Next, the phage library was incubated with the antigen coated well under proper conditions, and phages displaying the high affinity VHH was captured by the presented antigens. Non-binding and non-specific binding phages were then removed by extensive washing. Further, the bound phages were eluted by enzymatic digestion or other harsh elution buffers. Finally, the helper phages were added to the well to assist the amplification of eluted phages in E. coli. Phage library panning was performed up to 4 rounds or until phage ELISA demonstrates that binders are significantly enriched. The isolated positive phages were lysed and sequenced to obtain the VHH sequences.

Phage plaques from each round screening were randomly picked and amplified for the ELISA testing. In brief, 1 μg/ml β-catenin protein was coated onto an ELISA plate at 4° C. overnight, followed by the blocking with 5% MPBS for 1 h at 37′C. Purified monoclonal phages (50 μL), were added to the plate and incubated for 2 h at room temperature. After 6 washes with TBST buffer, horseradish peroxidase (HRP)-conjugated anti-M13 monoclonal antibody (1:10000 dilution) was added to the plate and incubation was continued at room temperature for 45 mins. Following an additional 6 washes with TBST buffer, 50 μl TMB substrate was added to the plate, and incubated for 10 min at 37° C. Finally, 50 μL 2 M sulfuric acid was added to stop the reaction. OD450 was measured using a microplate reader as the readout. An absorbance value >0.5 will be considered as positive.

After phage-ELISA detection, positive phages were selected from each library for DNA sequencing. Twelve select sdAbs were selected (BC3-BC14). The full sequences and the CDRs of each of BC3-BC14 are shown in Tables 4B and 4C.

Generation of VHH sdAb Against β-Catenin from Single B Cell Screening

From the immunized llama or alpaca, peripheral Blood Mononuclear Cells (PBMCs) will be obtained from β-catenin immunized animal with good titer determined by ELISA assay. Cells will be stained with a set of fluorophore conjugated antibodies to obtain antigen specific individual B cell through flow cytometry. Single B cell will be sorted into single well and cultured in the 96-well format. After culture period, cell culture supernatant from individual well will be evaluated by ELISA experiment to assess the positive binders. After that, VHH gene from selected positive single B cell will be amplified via standard RT-PCR. In brief, total RNA from the cell will be extracted from the lysed cells, and cDNA will be obtained by reverse transcription. Using cDNA as the template, antigen specific VHH sequences will be amplified through PCR with specific primer sets. VHH sequences will be analyzed by aligning with the IMGT database.

Generation of VHH Nanobody Against f-Catenin from Pichia Surface Display

The immunized BC2 library (Alpaca) that are cloned into Pichia surface display vector at NheI-BamHI sites will be transformed into Y24 (Fc displaying strain) by electroporation. The transformants will be selected on either G418 or URA drop-out plates. For the URA drop-out selection, the PpURA6 in the strain Y24 will be knocked out to generate a URA6 auxotroph. For Pichia surface display of the VHH library, the transformants will be grown in BMGY (1% yeast extract, 2% peptone, 100 mM potassium phosphate buffer, 1.34% yeast nitrogen base, 0.4 ug/mL biotin, and 1% glycerol, pH6.0) and induced in BMMY (same as BMGY except 1% methanol instead of 1% glycerol, pH6.0). The induced cells (VHH displayed) will be sorted using magnetic-activated cell sorting (MACS) to collect all binders. The binders will be sorted and enriched using fluorescence-activated cell sorting (FACS) to collect the binders that are specific to the BC2 antigen, followed by their characterization.

### Example 6: Methods for Generating of VHH sdAbs Against Ras Proteins

Single domain antibodies may be developed for binding various Ras proteins using methods similar to those described in Example 5.

### Example 7: Methods for Generating of VHH sdAbs Against MDM2 or MDM4 Proteins

Single domain antibodies may be developed for binding MDM2 and/or MDM4 proteins using methods similar to those described in Example 5.

### Example 8: Methods for Generating of VHH sdAbs Against ACS Proteins

Single domain antibodies may be developed for binding ACS proteins using methods similar to those described in Example 5.

### Example 9: Methods for Evaluating the PK, TK, and Biodistribution of Degradation Compounds Against β-Catenin Proteins in Wild Type Rodent Species

To test the PK of BC2, BC2 fusions, and their EEV conjugates, Balb/c, C57BL/6, and/or CD1 mice will be dosed via IV and SC routes. Each dose will be administered one time at the beginning of the experiment. Blood samples will be collected at the indicated time points. Tissue samples will be collected at the endpoint after euthanasia. The dosage will be determined based on the previous in vivo studies. For IV injection, 50 μl blood samples will be collected from individual mice at selected time points: 0.5 h, 2 h, 8 h, 24 h, 48 h, and 72 h from the submandibular vein using a lancet. For SC injection, 50 μl blood samples will be collected at 1 h, 8 h, 24 h, 48 h and 72 h. Multiple groups of animals will be used to reduce the blood collections from individual animals. Animals will be euthanized after the 72 h timepoint, and tissues and organs will be collected.

To further validate the pharmacokinetics (PK) data obtained from mice studies, five compounds tested in mice will also be tested in rats. Rats will be dosed one time via IV or SC, and 100 μL blood samples will be collected from individual rats at indicated time points: 0.5 h, 8 h, 24 h, 48 h, 72 h and 96 h. Rats will be euthanized after the 96 hour timepoint and tissues and organs will be collected. To evaluate the PK of a few development candidates at different dosages to evaluate the exposure and half-life, three different dosages of the investigated compounds will be tested.

To study the toxicokinetics (TK) of compounds after single injection of BC2, BC2 fusion, and their EEV conjugates, vehicle and degradation compounds will be administered through IV and SC routes. Mice and rats will be treated once with 4 different doses. The proposed low dose in the mice or rats will be 0.5 mpk, which is equivalent to the efficacious dose observed in vitro cellular target modulation system. The high dose is planned to be 50 mpk, which will be 100-fold higher than the efficacy dose. Two additional dosages between the high and low dose will be included to obtain more information on the toxicokinetics. Toxicokinetic sampling of plasma samples will occur pre-dose and daily for 7 days following dosing to determine the systemic exposure of K19. Anti-drug antibody samples will be collected and archived for potential future analysis at pre-study and end of the study. This study will be used to design and set doses for the future sub-chronic and efficacy studies. Animals will be sacrificed at 7 days after dosing.

Athymic nu/nu mice are widely used as a Xenograft model to detect the anti-tumor efficacy of drugs. Mice will be anesthetized using the Isoflurane vaporizer before tumor inoculation. For SNU398, HCT-116, SW480, DLD1, 786-0 or A549 cell lines, 5×10⁶ cells in 0.1 mL of 1:1 Matrigel:PBS will be injected subcutaneously into the right lateral flank of athymic nude mice to inoculate the cancer cells. Treatment cohorts will be derived through inoculation of human tumor cell lines generated in the above experiment. In all six-tumor models, when mean tumor volume reaches ˜100-150 mm³, mice will be randomized into treatment groups and receive either vehicle or β-catenin intrabody compounds up to twice per week. To prevent acute local injection site reaction, mice will be pre-injected i.p., if necessary, with 10 mg/kg of histamine antagonist, diphenhydramine. Dosage will be determined based on TK studies.

Mice will be treated for a total of 21 days or until tumor is resolved or reaches pre-determined tumor volume as per IACUC policy guidance, whichever comes first. IV, and/or IP will be tested. Vehicles (10% DMSO in PBS) will be used for control group. Tumor volume will be monitored by caliper measurement, and mouse body weights will be recorded. At days 1, 8, 15 and 21 days of treatment, blood will be collected to check exposure and other biomarkers of interests. At 21 days, mice will be euthanized, and tumor tissues will be collected and divided into two parts. One will be fixed to study the intracellular distribution of compounds by immunohistochemistry (IHC), and the other part will be quick frozen for ELISA, Western blot and/or qPCR assays.

To construct syngeneic cancer models, the mouse will be anesthetized using the Isoflurane vaporizer before tumor inoculation. Cultured MC38 colon cancer and Hepa1-6 liver cancer cells will be harvested, re-suspended in PBS at 1×10⁶ cells/ml with viability of >90%, and subcutaneously implanted in the right flank of female, 6- to 8-week, pathogen-free host mice (1×10⁶ cells/ml). Treatment cohorts will be derived through inoculation of MC38 or Hepa1-6 cells generated in the above experiment. The detailed treatment will be similar to the Xenograft model, and we will test 5 different compounds. When mean tumor volume reaches ˜100 mm³, tumor-bearing mice will be randomized into treatment groups and receive either vehicle or Entrada compounds on weekly bases (dosage regiment to be determined). Dosage will be based on TK studies. Tumor volume will be measured using digital calipers. Mice will be treated for a total of 21 days or until tumor is resolved, whichever comes first. IV and/or IP will be tested. Vehicles (e.g. saline or formulation buffer) will be used for control group. At days 1, 8, 15 and 21 days of treatment, blood will be collected to check exposure and other biomarkers of interests. At 21 days, mice will be euthanized, and tumor/organ samples will be collected from selected mice at the end of the study for biodistribution or biomarkers assay. Blood will be collected to check terminal PK exposure and other biomarkers of interests. Efficacy evaluation includes tumor volume, biomarker assays, as well as on/off target engagement assays.

### Example 10: Methods for Evaluating the PK, TK, and Biodistribution of Degradation Compounds Against RAS Proteins in Rodent Species

Degradation compounds targeting RAS may be evaluated using methods similar to Example 9.

### Example 11: Methods for Evaluating the PK, TK, and Biodistribution of Degradation Compounds Against ACS Proteins in Rodent Species

Degradation compounds targeting ACS may be evaluated using methods similar to Example 9.

### Example 12: Methods for Evaluating the PK, TK, and Biodistribution of Degradation Compounds Against MDM2 and MDM4 in Rodent Species

Degradation compounds targeting MDM2 and/or MDM4 may be evaluated using methods similar to Example 9.

### Example 13: Evaluation of Various MDM2 and MDM4 Targeting Degradation Compounds

Various MDM2 and MDM4 degradation constructs where designed, produced, and evaluated for targeting MDM2 and MDM4.

Plasmids encoding human GST-His-Thrombin-hMDM2 (GST-MDM2) or GST-His-Thrombin-hMDM4 (GAT-MDM4) were cloned into the pET-41a vector via de novo gene synthesis and recumbently produced in E. coli using methods similar to those described herein above. Cells were lysed and the protein was purified using glutathione agarose.

MDM2/4-Obstructing Protein constructs MOP3 and MOP3+ were designed based on loop insertions into a stability-optimized fibronectin framework identified as ¹⁰FN3 (Table 24 shows the specific amino acid and DNA sequences used). MOP3 and MOP3+ were engineered from ¹⁰FN3 through the insertion of a peptide sequence, SFAEYWALLS, into the FG loop (MOP3) or both the FG and CD loops (MOP3+) that has been shown to inhibit the interaction between MDM2/4 and p53 by binding to the p53 recognition site on MDM2/4. Intracellular expression of MOP3+ increased p53 activation, supporting its ability to interfere with MDM2/4-p53 binding (Lau, S-Y., et al. Protein Eng. Des. Sel. (2018), 31(7-8), 301-312.).

Plasmids encoding His-MOP3 (MOP3) or His-MOP3+ (MOP3+) were cloned into the pET-22b vector via de novo gene synthesis and recumbently produced in E. coli using methods similar to those described herein above. Cells were lysed and the protein was purified using glutathione agarose. Protein purity was determined by SDS-PAGE to be >90%.

TABLE 24
Degradation construct design

| | SEQ | | SEQ | |
|---|---|---|---|---|
| | ID | | ID | |
| ID | NO | Sequence | NO: | Sequence |
| GST- | 438 | MSPILGYWKIKGLVQ | 440 | ATGAGCCCAATTCTGGGTTACTGGAAGATCAAAGGTC |
| MDM2 | | PTRLLLEYLEEKYEE | | TGGTTCAGCCGACCCGTCTGCTGCTGGAATATCTGGA |
| | | HLYERDEGDKWRNKK | | AGAGAAATATGAAGAACACCTGTATGAGCGTGATGAA |
| | | FELGLEFPNLPYYID | | GGTGATAAATGGCGTAACAAGAAATTTGAACTGGGTC |
| | | GDVKLTQSMAIIRYI | | TGGAATTTCCTAACCTGCCGTACTACATTGACGGTGA |
| | | ADKHNMLGGCPKERA | | CGTGAAACTGACCCAGTCCATGGCAATCATCCGTTAC |
| | | EISMLEGAVLDIRYG | | ATTGCAGACAAACATAACATGCTGGGTGGTTGCCCGA |
| | | VSRIAYSKDFETLKV | | AAGAACGTGCGGAAATTTCCATGCTGGAAGGTGCGGT |
| | | DFLSKLPEMLKMFED | | CCTGGACATTCGTTATGGCGTAAGCCGTATCGCTTAC |
| | | RLCHKTYLNGDHVTH | | TCTAAAGATTTTGAGACCCTGAAAGTTGATTTTCTGA |
| | | PDFMLYDALDVVLYM | | GCAAACTGCCGGAAATGCTGAAAATGTTCGAGGACCG |
| | | DPMCLDAFPKLVCFK | | CCTGTGCCACAAAACTTACCTGAACGGTGATCACGTT |
| | | KRIEAIPQIDKYLKS | | ACCCACCCTGACTTCATGCTGTACGATGCTCTGGACG |
| | | SKYIAWPLQGWQATF | | TTGTACTGTACATGGACCCGATGTGCCTGGACGCATT |
| | | GGGDHPPKSDGSTSG | | TCCGAAGCTGGTTTGCTTTAAAAAACGCATCGAAGCG |
| | | SGHHHHHHSAGLVPR | | ATCCCGCAAATTGACAAATATCTGAAAAGCTCTAAAT |
| | | GSMCNTNMSVPTDGA | | ACATTGCTTGGCCACTGCAGGGTTGGCAGGCTACCTT |
| | | VTTSQIPASEQETLV | | CGGTGGTGGTGACCACCCACCTAAAAGCGATGGCTCT |
| | | RPKPLLLKLLKSVGA | | ACTTCCGGCTCCGGCCATCACCACCATCACCACTCTG |
| | | QKDTYTMKEVLFYLG | | CTGGCCTGGTTCCTCGTGGTTCTATGTGTAACACCAA |
| | | QYIMTKRLYDEKQQH | | TATGTCTGTGCCGACCGACGGTGCAGTGACTACCTCC |
| | | IVYCSNDLLGDLFGV | | CAGATCCCGGCGTCCGAACAGGAGACCCTGGTTCGTC |
| | | PSFSVKEHRKIYTMI | | CGAAACCGCTGCTGCTGAAACTGCTGAAAAGCGTAGG |
| | | YRNLVVV | | TGCGCAGAAAGACACCTATACCATGAAAGAAGTGCTG |
| | | | | TTTTATCTGGGCCAGTATATCATGACTAAGCGTCTGT |
| | | | | ACGACGAAAAACAGCAGCATATCGTATACTGTTCTAA |
| | | | | CGACCTGCTGGGTGACCTGTTCGGCGTACCAAGCTTC |
| | | | | TCTGTTAAAGAACACCGTAAAATCTATACTATGATTT |
| | | | | ACCGCAACCTGGTAGTAGTG |
| GST- | 439 | MSPILGYWKIKGLVQ | 441 | ATGTCTCCAATCCTGGGCTACTGGAAAATCAAAGGCC |
| MDM4 | | PTRLLLEYLEEKYEE | | TGGTGCAACCAACCCGTCTGCTGCTGGAATATCTGGA |
| | | HLYERDEGDKWRNKK | | AGAAAAATACGAGGAACATCTGTATGAACGTGACGAA |
| | | FELGLEFPNLPYYID | | GGCGACAAATGGCGCAACAAAAAATTTGAACTGGGTC |
| | | GDVKLTQSMAIIRYI | | TGGAGTTCCCGAACCTGCCGTACTACATCGATGGTGA |
| | | ADKHNMLGGCPKERA | | CGTTAAACTGACGCAAAGCATGGCGATTATTCGTTAC |
| | | EISMLEGAVLDIRYG | | ATTGCCGATAAACACAACATGCTGGGTGGCTGTCCGA |
| | | VSRIAYSKDFETLKV | | AGGAACGTGCTGAAATCTCTATGCTGGAAGGTGCAGT |
| | | DFLSKLPEMLKMFED | | GCTGGATATTCGTTACGGTGTTTCTCGCATTGCCTAC |
| | | RLCHKTYLNGDHVTH | | TCCAAGGACTTCGAGACCCTGAAAGTCGACTTTCTGT |
| | | PDFMLYDALDVVLYM | | CTAAACTGCCGGAAATGCTGAAAATGTTCGAAGACCG |
| | | DPMCLDAFPKLVCFK | | TCTGTGCCACAAAACCTACCTGAACGGCGATCACGTC |
| | | KRIEAIPQIDKYLKS | | ACCCATCCGGACTTCATGCTGTACGACGCTCTGGACG |
| | | SKYIAWPLQGWQATF | | TTGTGCTGTATATGGATCCTATGTGCCTGGACGCCTT |
| | | GGGDHPPKSDGSTSG | | CCCGAAACTGGTATGCTTCAAGAAACGTATCGAAGCT |
| | | SGHHHHHHSAGLVPR | | ATTCCGCAGATCGATAAATACCTGAAATCTTCTAAAT |
| | | GSMTSFSTSAQCSTS | | ACATTGCTTGGCCGCTGCAGGGTTGGCAAGCAACCTT |
| | | DSACRISPGQINQVR | | TGGCGGTGGCGATCACCCTCCGAAATCCGACGGCTCC |
| | | PKLPLLKILHAAGAQ | | ACTTCTGGCTCTGGTCACCACCACCACCACCACTCCG |
| | | GEMFTVKEVMHYLGQ | | CAGGTCTGGTCCCTCGTGGTTCCATGACTTCTTTTTC |
| | | YIMVKQLYDQQEQHM | | CACTTCTGCTCAGTGTTCTACCAGCGATAGCGCGTGC |
| | | VYCGGDLLGELLGRQ | | CGTATCTCCCCGGGTCAGATCAACCAGGTACGTCCGA |
| | | SFSVKDPSPLYDMLR | | AACTGCCGCTGCTGAAGATCCTGCATGCTGCTGGCGC |
| | | KNLVTLATATTDAAQ | | GCAAGGCGAAATGTTTACCGTTAAAGAAGTGATGCAC |
| | | TLALAQDHSMDIPSQ | | TACCTGGGTCAGTACATCATGGTCAAACAGCTGTATG |
| | | DQLKQSAEESSTSRK | | ATCAACAGGAACAGCATATGGTATATTGCGGTGGTGA |
| | | RTTEDDIPTLPTSEH | | TCTGCTGGGTGAACTGCTGGGTCGCCAATCTTTTTCC |
| | | KCIHSREDEDLIENL | | GTTAAAGATCCTTCTCCTCTGTATGACATGCTGCGCA |
| | | | | AAAACCTGGTGACCCTGGCGACCGCAACTACTGACGC |
| | | | | TGCACAAACTCTGGCCCTGGCCCAAGACCACTCTATG |
| | | | | GACATTCCGTCTCAGGATCAACTGAAGCAGTCCGCCG |
| | | | | AGGAAAGCAGCACCAGCCGTAAACGTACCACCGAGGA |
| | | | | TGATATTCCGACCCTGCCGACCAGCGAACACAAATGC |
| | | | | ATTCACAGCCGCGAAGACGAGGACCTGATCGAAAATC |
| | | | | TG |
| MOP3 | 398 | VSDVPRKLEVVAATP | 409 | ATATGGTGAGCGACGTGCCGCGTAAACTGGAAGTGGT |
| | | TSLLISWDAPAVTVR | | TGCGGCGACCCCGACCAGCCTGCTGATTAGCTGGGAT |
| | | YYRITYGETGGNSPV | | GCGCCGGCGGTGACCGTGCGTTACTATCGTATCACCT |
| | | QEFTVPGSKSTATIS | | ACGGCGAGACCGGTGGCAACAGCCCGGTGCAGGAATT |
| | | GLKPGVDYTITVYAV | | CACCGTTCCGGGTAGCAAGAGCACCGCGACCATCAGC |
| | | TSGGGSFAEYWALLS | | GGCCTGAAACCGGGTGTGGACTACACCATTACCGTGT |
| | | GGGSPISINYRTALE | | ATGCGGTTACCAGCGGTGGCGGTAGCTTTGCGGAGTA |
| | | | | TTGGGCGCTGCTGAGCGGCGGCGGTAGCCCGATTAGC |
| | | | | ATTAACTATCGCACCGCGCTGGAATAAGGATCC |
| MOP3+ | 137 | VSDVPRKLEVVAATP | 410 | CATATGGTGAGCGACGTGCCGCGTAAACTGGAAGTGG |
| | | TSLLISWDAPAVTVR | | TTGCGGCGACCCCGACCAGCCTGCTGATTAGCTGGGA |
| | | YYRITYGETSGGGSF | | TGCGCCGGCGGTGACCGTGCGTTACTATCGTATCACC |
| | | AEYWALLSGGGSVQE | | TACGGCGAGACCAGCGGTGGCGGTAGCTTCGCGGAAT |
| | | FTVPGSKSTATISGL | | ATTGGGCGCTGCTGAGCGGCGGTGGCAGCGTGCAGGA |
| | | KPGVDYTITVYAVTS | | GTTTACCGTTCCGGGTAGCAAGAGCACCGCGACCATC |
| | | GGGSFAEYWALLSGG | | AGCGGCCTGAAACCGGGTGTGGACTACACCATTACCG |
| | | GSPISINYRTALE | | TGTATGCGGTTACCAGCGGTGGCGGTAGCTTTGCGGA |
| | | | | ATACTGGGCGCTGCTGAGCGGCGGCGGTAGCCCGATT |
| | | | | AGCATTAACTATCGCACCGCGCTGGAATAAGGATCC |

Biophysical Characterizations Unconjugated and conjugatedMOP3 and MOP3+

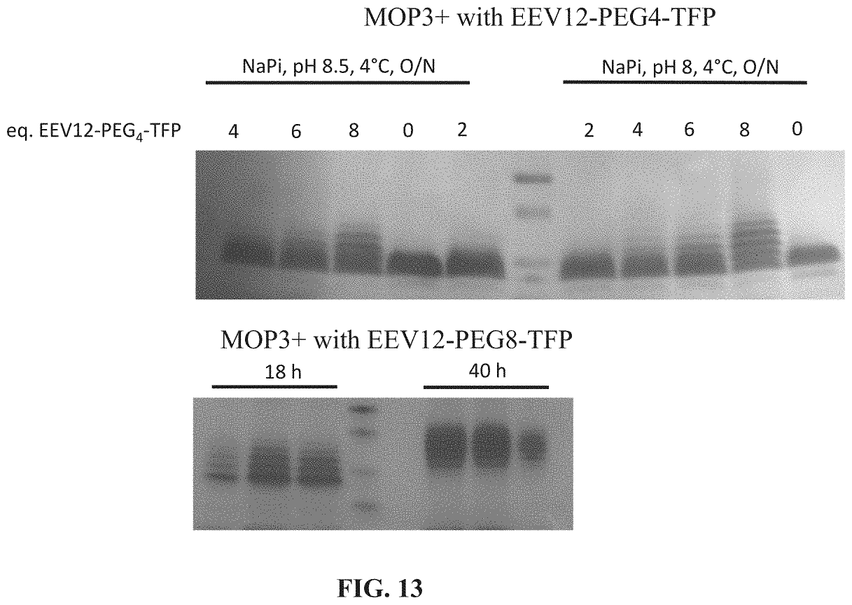

MOP3 and MOP3+ where conjugated to EEV12 PEG4 and EEV12-PEG8 (cyclo(FfΦRrRrQ)-PEG-TFP using tetrafluorophenol (TFP) activated ester-primary amine chemistry. MOP3 or MOP3+ was mixed with various equivalents of EEV12-PEG4-TFP or EEV12-PEG8-TFP. Specifically, various equivalents of EEV12-PEG4-TFP was mixed MOP3+ in sodium phosphate at pH 8.5 and allowed to react overnight at 4° C. Ten equivalents EEV12-PEF8-TFP was mixed with MOP3+ in a solution of 50 mM sodium phosphate, 150 mM NaCl, 2 mM DTT at pH8.5 and allowed to react for 18 hours or 40 hours. Reaction progress was monitored via SDS-PAGE. An increase in molecular weight from was observed from unconjugated to the conjugated product indicating conjugation did occur (FIG. 13). Multiple bands are observed due to conjugations to more than one lysine on the MOP3 ad MP3+.

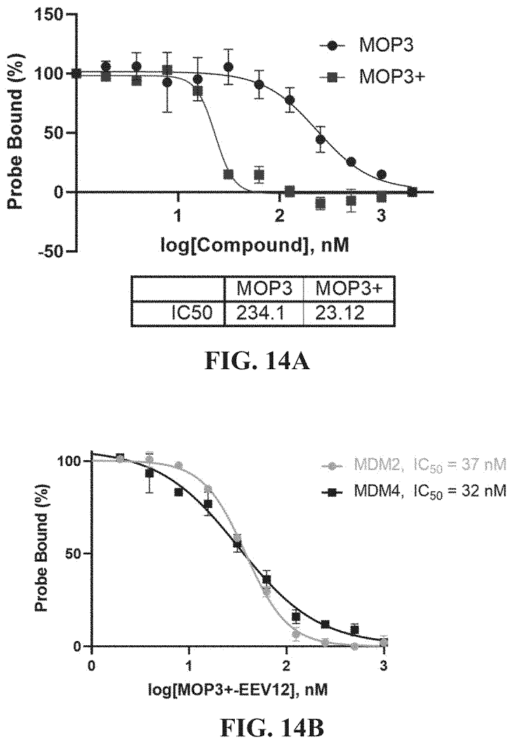

Determination of the binding affinity of MOP3 and MOP3+ to GST-MDM2 or GST-MDM4 was performed using competitive fluorescence polarization. 10 nM GST-MDM2 or GST-MDM4 were incubated with 10 nM fluorescently-labeled probe peptide that binds to MDM2 with a K_{D}=10 nM in PBS supplemented with 0.01% Triton-X100 and incubated with gentle mixing for 1 h at RT. Two-fold serial dilutions of MOP3 or MOP3+ were prepared in PBS+0.01% Triton-X100 and aliquots of the equilibrated fluorescent probe-MDM2/MDM4 complex were added to each dilution and incubated for an additional 1 h at RT with gentle mixing. After 1 h, fluorescence polarization was measured using a SpectraMax iD5 (Molecular Devices and IC₅₀ was determined. MOP3+ (IC₅₀=233.12 nM) demonstrated a ˜10-fold improvement in binding affinity relative to MOP3 (IC₅₀=234.1 nM) (FIG. 14A). MOP3+-EEV12 maintained binding to GST-MDM2 (IC₅₀=37 nM) analogous to the unconjugated species and had equivalent binding to GST-MDM4 (IC₅₀=32 nM) (FIG. 14B).

### Evaluations of In Vitro Pharmacological Effects of MOP3 and its EEV Conjugates

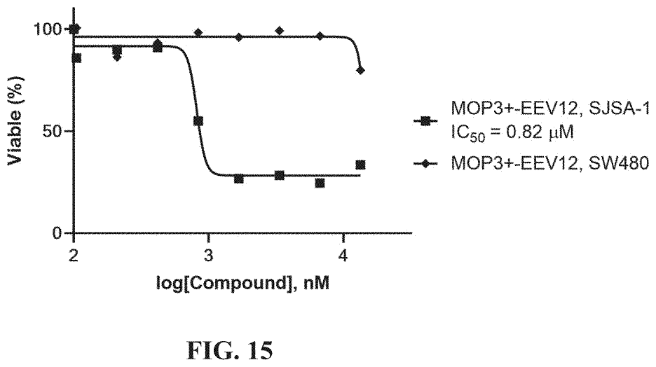

The effects of MOP3, MOP3+, MOP3-EEV12, and MOP3+-EEV12 on cell viability of SW480 (p53 mutant) and SJSA-1 (p53 wild type) cell lines was determined using methods similar to Example 2. Conjugated MOP3+-EEV12 was able to reduce the viability of p53^{WT}, MDM2/4 overexpressing SJSA-1 cells, while having no effects on a p53^{-/-} cell line, suggesting a p53-dependent mechanism of action (FIG. 15).

### Example 14: Evaluation of Various NLRP3 Targeting Degradation Constructs and Degradation Compounds

Various NLRP3 targeting degradation constructs and degradation compounds were designed, produced, and evaluated for targeting NLRP3 proteins.

Sequences of human full length NLRP3, human NLRP3 PYD domain, mouse NLRP3 and mouse NLRP3 domain, each with N terminal His+flag Tag were optimized, synthesized and subcloned into vector pET28a and recombinantly produced in E. coli similar to methods described hereinabove. Table T14 shows the protein and DNA sequences used. Cells were lysed and the protein was isolated and purified using Ni affinity chromatography. The protein purity and molecular weight were determined by standard SDS-PAGE along with Western blot confirmation using mouse-anti-His mAb and rabbit anti-FLAG pAb.

Human NLRP3:
(SEQ ID NO: 442)
MHHHHHHDYKDDDDKKMASTRCKLARYLEDLEDVDLKKFKMHLEDYPPQKGCIPLPRGQTEKAD
HVDLATLMIDFNGEEKAWAMAVWIFAAINRRDLYEKAKRDEPKWGSDNARVSNPTVICQEDSIE
EEWMGLLEYLSRISICKMKKDYRKKYRKYVRSRFQCIEDRNARLGESVSLNKRYTRLRLIKEHR
SQQEREQELLAIGKTKTCESPVSPIKMELLFDPDDEHSEPVHTVVFQGAAGIGKTILARKMMLD
WASGTLYQDRFDYLFYIHCREVSLVTQRSLGDLIMSCCPDPNPPIHKIVRKPSRILFLMDGFDE
LQGAFDEHIGPLCTDWQKAERGDILLSSLIRKKLLPEASLLITTRPVALEKLQHLLDHPRHVEI
LGFSEAKRKEYFFKYFSDEAQARAAFSLIQENEVLFTMCFIPLVCWIVCTGLKQQMESGKSLAQ
TSKTTTAVYVFFLSSLLQPRGGSQEHGLCAHLWGLCSLAADGIWNQKILFEESDLRNHGLQKAD
VSAFLRMNLFQKEVDCEKFYSFIHMTFQEFFAAMYYLLEEEKEGRTNVPGSRLKLPSRDVTVLL
ENYGKFEKGYLIFVVRFLFGLVNQERTSYLEKKLSCKISQQIRLELLKWIEVKAKAKKLQIQPS
QLELFYCLYEMQEEDFVQRAMDYFPKIEINLSTRMDHMVSSFCIENCHRVESLSLGFLHNMPKE
EEEEEKEGRHLDMVQCVLPSSSHAACSHGLVNSHLTSSFCRGLFSVLSTSQSLTELDLSDNSLG
DPGMRVLCETLQHPGCNIRRLWLGRCGLSHECCFDISLVLSSNQKLVELDLSDNALGDFGIRLL
CVGLKHLLCNLKKLWLVSCCLTSACCQDLASVLSTSHSLTRLYVGENALGDSGVAILCEKAKNP
QCNLQKLGLVNSGLTSVCCSALSSVLSTNQNLTHLYLRGNTLGDKGIKLLCEGLLHPDCKLQVL
ELDNCNLTSHCCWDLSTLLTSSQSLRKLSLGNNDLGDLGVMMFCEVLKQQSCLLQNLGLSEMYF
NYETKSALETLQEEKPELTVVFEPSW
Human NLRP3:
(SEQ ID NO: 443)
ATGCATCACCACCACCACCACGATTACAAGGATGATGATGATAAGAAGATGGCGAGCACCCGTT
GCAAGCTGGCGCGTTATCTGGAAGACCTGGAGGACGTTGATCTGAAGAAATTCAAAATGCACCT
GGAAGATTACCCGCCGCAGAAGGGTTGCATCCCGCTGCCGCGTGGCCAAACCGAGAAAGCGGAC
CACGTTGATCTGGCGACCCTGATGATTGACTTCAACGGCGAGGAAAAGGCGTGGGCGATGGCGG
TGTGGATCTTTGCGGCGATTAACCGTCGTGATCTGTATGAAAAGGCGAAACGTGACGAGCCGAA
ATGGGGTAGCGATAACGCGCGTGTGAGCAACCCGACCGTTATCTGCCAGGAAGACAGCATTGAG
GAAGAGTGGATGGGCCTGCTGGAATACCTGAGCCGTATCAGCATTTGCAAGATGAAGAAAGACT
ACCGTAAGAAATACCGTAAGTATGTTCGTAGCCGTTTCCAGTGCATCGAGGATCGTAACGCGCG
TCTGGGTGAAAGCGTGAGCCTGAACAAACGTTACACCCGTCTGCGTCTGATTAAGGAGCACCGT
AGCCAGCAAGAGCGTGAACAAGAGCTGCTGGCGATCGGCAAGACCAAAACCTGCGAGAGCCCGG
TTAGCCCGATTAAGATGGAACTGCTGTTCGACCCGGACGATGAACACAGCGAGCCGGTGCACAC
CGTGGTTTTTCAGGGTGCGGCGGGTATCGGCAAGACCATTCTGGCGCGTAAAATGATGCTGGAT
TGGGCGAGCGGCACCCTGTACCAGGACCGTTTCGATTACCTGTTTTATATCCACTGCCGTGAAG
TGAGCCTGGTTACCCAACGTAGCCTGGGTGACCTGATTATGAGCTGCTGCCCGGATCCGAACCC
GCCGATCCACAAGATTGTTCGTAAACCGAGCCGTATCCTGTTCCTGATGGACGGTTTTGATGAG
CTGCAGGGCGCGTTCGACGAACACATTGGTCCGCTGTGCACCGACTGGCAAAAAGCGGAGCGTG
GCGATATCCTGCTGAGCAGCCTGATTCGTAAGAAACTGCTGCCGGAGGCGAGCCTGCTGATCAC
CACCCGTCCGGTGGCGCTGGAAAAACTGCAGCACCTGCTGGACCACCCGCGTCACGTTGAGATT
CTGGGTTTTAGCGAAGCGAAGCGTAAAGAGTACTTCTTTAAGTATTTCAGCGATGAAGCGCAGG
CGCGTGCGGCGTTTAGCCTGATCCAAGAAAACGAGGTGCTGTTCACCATGTGCTTTATCCCGCT
GGTGTGCTGGATTGTTTGCACCGGTCTGAAGCAGCAAATGGAAAGCGGCAAAAGCCTGGCGCAG
ACCAGCAAGACCACCACCGCGGTGTACGTTTTCTTTCTGAGCAGCCTGCTGCAACCGCGTGGTG
GCAGCCAAGAACATGGTCTGTGCGCGCACCTGTGGGGCCTGTGCAGCCTGGCGGCGGACGGTAT
CTGGAACCAAAAAATTCTGTTCGAAGAGAGCGACCTGCGTAACCACGGCCTGCAGAAGGCGGAT
GTGAGCGCGTTCCTGCGTATGAACCTGTTTCAAAAGGAAGTTGATTGCGAGAAATTCTATAGCT
TTATCCACATGACCTTTCAGGAATTCTTTGCGGCGATGTACTATCTGCTGGAAGAGGAAAAAGA
GGGTCGTACCAACGTGCCGGGTAGCCGTCTGAAGCTGCCGAGCCGTGACGTGACCGTTCTGCTG
GAAAACTACGGCAAGTTCGAGAAAGGCTATCTGATTTTTGTGGTTCGTTTCCTGTTTGGTCTGG
TGAACCAGGAACGTACCAGCTACCTGGAGAAGAAACTGAGCTGCAAAATCAGCCAGCAAATTCG
TCTGGAACTGCTGAAGTGGATCGAGGTTAAGGCGAAAGCGAAGAAACTGCAGATTCAACCGAGC
CAACTGGAGCTGTTCTACTGCCTGTATGAAATGCAGGAAGAGGACTTCGTGCAACGTGCGATGG
ATTATTTTCCGAAGATCGAAATTAACCTGAGCACCCGTATGGACCACATGGTTAGCAGCTTCTG
CATCGAAAACTGCCACCGTGTTGAGAGCCTGAGCCTGGGTTTTCTGCACAACATGCCGAAAGAG
GAAGAGGAAGAGGAAAAAGAGGGTAGACACCTGGATATGGTGCAATGCGTTCTGCCGAGCAGCA
GCCATGCGGCGTGCAGCCACGGTCTGGTGAACAGCCACCTGACCAGCAGCTTCTGCCGTGGCCT
GTTTAGCGTTCTGAGCACCAGCCAGAGCCTGACCGAACTGGACCTGAGCGATAACAGCCTGGGT
GACCCGGGCATGCGTGTGCTGTGCGAGACCCTGCAACACCCGGGTTGCAACATCCGTCGTCTGT
GGCTGGGTCGTTGCGGCCTGAGCCACGAATGCTGCTTCGACATTAGCCTGGTGCTGAGCAGCAA
CCAAAAACTGGTTGAGCTGGATCTGAGCGACAACGCGCTGGGTGATTTTGGCATCCGTCTGCTG
TGCGTGGGTCTGAAGCACCTGCTGTGCAACCTGAAGAAACTGTGGCTGGTTAGCTGCTGCCTGA
CCAGCGCGTGCTGCCAGGACCTGGCGAGCGTGCTGAGCACCAGCCACAGCCTGACCCGTCTGTA
CGTGGGCGAAAACGCGCTGGGTGATAGCGGCGTTGCGATCCTGTGCGAGAAGGCGAAAAACCCG
CAGTGCAACCTGCAAAAACTGGGTCTGGTGAACAGCGGTCTGACCAGCGTTTGCTGCAGCGCGC
TGAGCAGCGTTCTGAGCACCAACCAGAACCTGACCCACCTGTATCTGCGTGGTAACACCCTGGG
TGACAAGGGCATCAAACTGCTGTGCGAAGGCCTGCTGCACCCGGACTGCAAGCTGCAGGTGCTG
GAGCTGGATAACTGCAACCTGACCAGCCACTGCTGCTGGGATCTGAGCACCCTGCTGACCAGCA
GCCAAAGCCTGCGTAAACTGAGCCTGGGTAACAACGACCTGGGTGATCTGGGCGTGATGATGTT
CTGCGAGGTTCTGAAGCAGCAAAGCTGCCTGCTGCAGAACCTGGGCCTGAGCGAAATGTACTTT
AACTATGAGACCAAAAGCGCGCTGGAAACCCTGCAAGAGGAAAAGCCGGAGCTGACCGTGGTTT
TCGAACCGAGCTGGTAATGAAAGCTT
Mouse NLRP3:
(SEQ ID NO: 444)
MHHHHHHDYKDDDDKTSVRCKLAQYLEDLEDVDLKKFKMHLEDYPPEKGCIPVPRGQMEKADHL
DLATLMIDFNGEEKAWAMAVWIFAAINRRDLWEKAKKDQPEWNDTCTSHSSMVCQEDSLEEEWM
GLLGYLSRISICKKKKDYCKMYRRHVRSRFYSIKDRNARLGESVDLNSRYTQLQLVKEHPSKQE
REHELLTIGRTKMRDSPMSSLKLELLFEPEDGHSEPVHTVVFQGAAGIGKTILARKIMLDWALG
KLFKDKFDYLFFIHCREVSLRTPRSLADLIVSCWPDPNPPVCKILRKPSRILFLMDGFDELQGA
FDEHIGEVCTDWQKAVRGDILLSSLIRKKLLPKASLLITTRPVALEKLQHLLDHPRHVEILGFS
EAKRKEYFFKYFSNELQAREAFRLIQENEVLFTMCFIPLVCWIVCTGLKQQMETGKSLAQTSKT
TTAVYVFFLSSLLQSRGGIEEHLFSDYLQGLCSLAADGIWNQKILFEECDLRKHGLQKTDVSAF
LRMNVFQKEVDCERFYSFSHMTFQEFFAAMYYLLEEEAEGETVRKGPGGCSDLLNRDVKVLLEN
YGKFEKGYLIFVVRFLFGLVNQERTSYLEKKLSCKISQQVRLELLKWIEVKAKAKKLQWQPSQL
ELFYCLYEMQEEDFVQSAMDHFPKIEINLSTRMDHVVSSFCIKNCHRVKTLSLGFFHNSPKEEE
EERRGGRPLDQVQCVFPDTHVACSSRLVNCCLTSSFCRGLFSSLSTNRSLTELDLSDNTLGDPG
MRVLCEALQHPGCNIQRLWLGRCGLSHQCCFDISSVLSSSQKLVELDLSDNALGDFGIRLLCVG
LKHLLCNLQKLWLVSCCLTSACCQDLALVLSSNHSLTRLYIGENALGDSGVQVLCEKMKDPQCN
LQKLGLVNSGLTSICCSALTSVLKTNQNFTHLYLRSNALGDTGLRLLCEGLLHPDCKLQMLELD
NCSLTSHSCWNLSTILTHNHSLRKLNLGNNDLGDLCVVTLCEVLKQQGCLLQSLQLGEMYLNRE
TKRALEALQEEKPELTIVFEISW
Mouse NLRP3:
(SEQ ID NO: 445)
ATGCATCACCACCACCACCACGATTACAAGGATGATGATGATAAGACAAGCGTGCGTTGCAAAC
TGGCGCAGTATCTGGAGGACCTGGAAGACGTTGATCTGAAGAAATTCAAAATGCACCTGGAGGA
TTACCCGCCGGAAAAGGGTTGCATCCCGGTTCCGCGTGGCCAAATGGAAAAAGCGGACCACCTG
GATCTGGCGACCCTGATGATTGACTTCAACGGCGAGGAAAAGGCGTGGGCGATGGCGGTGTGGA
TCTTTGCGGCGATTAACCGTCGTGATCTGTGGGAAAAGGCGAAGAAAGACCAGCCGGAGTGGAA
CGATACCTGCACCAGCCACAGCAGCATGGTTTGCCAAGAGGACAGCCTGGAGGAAGAGTGGATG
GGTCTGCTGGGCTATCTGAGCCGTATCAGCATTTGCAAGAAAAAGAAAGATTACTGCAAGATGT
ACCGTCGTCACGTGCGTAGCCGTTTCTACAGCATCAAGGACCGTAACGCGCGTCTGGGTGAAAG
CGTGGATCTGAACAGCCGTTATACCCAGCTGCAACTGGTTAAGGAGCACCCGAGCAAACAGGAA
CGTGAGCACGAACTGCTGACCATTGGCCGTACCAAAATGCGTGACAGCCCGATGAGCAGCCTGA
AGCTGGAACTGCTGTTCGAGCCGGAAGATGGTCACAGCGAGCCGGTTCACACCGTGGTTTTTCA
GGGCGCGGCGGGTATCGGCAAGACCATTCTGGCGCGTAAAATCATGCTGGACTGGGCGCTGGGT
AAACTGTTCAAGGACAAATTTGATTACCTGTTCTTTATCCACTGCCGTGAAGTGAGCCTGCGTA
CCCCGCGTAGCCTGGCGGACCTGATTGTTAGCTGCTGGCCGGATCCGAACCCGCCGGTTTGCAA
GATCCTGCGTAAACCGAGCCGTATTCTGTTCCTGATGGACGGTTTTGATGAGCTGCAGGGCGCG
TTCGACGAGCACATTGGTGAAGTGTGCACCGACTGGCAAAAAGCGGTTCGTGGCGATATCCTGC
TGAGCAGCCTGATTCGTAAGAAACTGCTGCCGAAAGCGAGCCTGCTGATCACCACCCGTCCGGT
GGCGCTGGAAAAGCTGCAGCACCTGCTGGATCACCCGCGTCACGTTGAGATTCTGGGTTTTAGC
GAGGCGAAGCGTAAGGAATACTTCTTTAAGTACTTCAGCAACGAGCTGCAGGCGCGTGAAGCGT
TTCGTCTGATCCAAGAGAACGAAGTTCTGTTCACCATGTGCTTTATCCCGCTGGTGTGCTGGAT
TGTTTGCACCGGTCTGAAGCAGCAAATGGAAACCGGCAAAAGCCTGGCGCAGACCAGCAAGACC
ACCACCGCGGTGTACGTTTTCTTTCTGAGCAGCCTGCTGCAAAGCCGTGGTGGCATTGAAGAGC
ACCTGTTCAGCGACTATCTGCAGGGTCTGTGCAGCCTGGCGGCGGATGGCATCTGGAACCAAAA
AATTCTGTTTGAAGAGTGCGACCTGCGTAAGCACGGTCTGCAGAAAACCGATGTGAGCGCGTTC
CTGCGTATGAACGTGTTTCAAAAAGAGGTGGACTGCGAACGTTTCTACAGCTTTAGCCACATGA
CCTTCCAGGAGTTCTTTGCGGCGATGTACTATCTGCTGGAAGAGGAAGCGGAGGGTGAAACCGT
TCGTAAAGGTCCGGGTGGCTGCAGCGACCTGCTGAACCGTGATGTGAAGGTTCTGCTGGAGAAC
TACGGCAAGTTCGAAAAAGGCTATCTGATCTTTGTGGTTCGTTTCCTGTTTGGCCTGGTGAACC
AGGAGCGTACCAGCTATCTGGAAAAGAAACTGAGCTGCAAAATCAGCCAGCAAGTGCGTCTGGA
GCTGCTGAAGTGGATTGAAGTTAAGGCGAAAGCGAAGAAACTGCAGTGGCAACCGAGCCAACTG
GAACTGTTCTACTGCCTGTATGAGATGCAGGAAGAGGACTTCGTTCAAAGCGCGATGGATCACT
TTCCGAAAATCGAAATTAACCTGAGCACCCGTATGGACCACGTGGTTAGCAGCTTTTGCATCAA
GAACTGCCACCGTGTGAAAACCCTGAGCCTGGGTTTCTTTCACAACAGCCCGAAGGAAGAGGAA
GAGGAGCGTCGTGGTGGCCGTCCGCTGGACCAGGTGCAATGCGTTTTTCCGGATACCCATGTGG
CGTGCAGCAGCCGTCTGGTTAACTGCTGCCTGACCAGCAGCTTCTGCCGTGGTCTGTTTAGCAG
CCTGAGCACCAACCGTAGCCTGACCGAACTGGACCTGAGCGATAACACCCTGGGTGATCCGGGC
ATGCGTGTGCTGTGCGAGGCGCTGCAGCACCCGGGTTGCAACATCCAACGTCTGTGGCTGGGTC
GTTGCGGCCTGAGCCACCAGTGCTGCTTCGACATTAGCAGCGTGCTGAGCAGCAGCCAAAAACT
GGTTGAGCTGGATCTGAGCGACAACGCGCTGGGTGATTTTGGCATCCGTCTGCTGTGCGTGGGT
CTGAAACACCTGCTGTGCAACCTGCAGAAGCTGTGGCTGGTTAGCTGCTGCCTGACCAGCGCGT
GCTGCCAAGACCTGGCGCTGGTGCTGAGCAGCAACCACAGCCTGACCCGTCTGTACATCGGCGA
AAACGCGCTGGGTGACAGCGGCGTGCAGGTTCTGTGCGAGAAGATGAAAGATCCGCAGTGCAAC
CTGCAAAAACTGGGTCTGGTTAACAGCGGCCTGACCAGCATTTGCTGCAGCGCGCTGACCAGCG
TGCTGAAGACCAACCAGAACTTCACCCACCTGTATCTGCGTAGCAACGCGCTGGGTGATACCGG
CCTGCGTCTGCTGTGCGAAGGTCTGCTGCACCCGGACTGCAAGCTGCAAATGCTGGAGCTGGAT
AACTGCAGCCTGACCAGCCACAGCTGCTGGAACCTGAGCACCATCCTGACCCACAACCACAGCC
TGCGTAAACTGAACCTGGGTAACAACGACCTGGGCGATCTGTGCGTGGTTACCCTGTGCGAGGT
TCTGAAGCAGCAAGGTTGCCTGCTGCAGAGCCTGCAACTGGGCGAGATGTACCTGAACCGTGAA
ACCAAACGTGCGCTGGAGGCGCTGCAGGAAGAGAAGCCGGAACTGACCATCGTGTTTGAGATTA
GCTGGTAATGAAAGCTT
Human NLRP3 PYD:
(SEQ ID NO: 446)
MHHHHHHDYKDDDDKKMASTRCKLARYLEDLEDVDLKKFKMHLEDYPPQKGCIPLPRGQTEKAD
HVDLATLMIDFNGEEKAWAMAVWIFAAINRRDLYEKAKRDEPK
Human NLRP3 PYD:
(SEQ ID NO: 447)
ATGCATCACCACCACCACCACGATTACAAGGATGATGATGATAAGAAGATGGCGAGCACCCGTT
GCAAGCTGGCGCGTTATCTGGAAGACCTGGAGGACGTTGATCTGAAGAAATTCAAAATGCACCT
GGAAGATTACCCGCCGCAGAAGGGTTGCATCCCGCTGCCGCGTGGCCAAACCGAGAAAGCGGAC
CACGTTGATCTGGCGACCCTGATGATTGACTTCAACGGCGAGGAAAAGGCGTGGGCGATGGCGG
TGTGGATCTTTGCGGCGATTAACCGTCGTGATCTGTATGAAAAGGCGAAACGTGACGAGCCGAA
ATAATGAAAGCTT
Mouse NLRP3 PYD:
(SEQ ID NO: 448)
MHHHHHHDYKDDDDKTSVRCKLAQYLEDLEDVDLKKFKMHLEDYPPEKGCIPVPRGQMEKADHL
DLATLMIDFNGEEKAWAMAVWIFAAINRRDLWEKAKKDQPE
Mouse NLRP3 PYD:
(SEQ ID NO: 449)
ATGCATCACCACCACCACCACGATTACAAGGATGATGATGATAAGACAAGCGTGCGTTGCAAAC
TGGCGCAGTATCTGGAGGACCTGGAAGACGTTGATCTGAAGAAATTCAAAATGCACCTGGAGGA
TTACCCGCCGGAAAAGGGTTGCATCCCGGTTCCGCGTGGCCAAATGGAAAAAGCGGACCACCTG
GATCTGGCGACCCTGATGATTGACTTCAACGGCGAGGAAAAGGCGTGGGCGATGGCGGTGTGGA
TCTTTGCGGCGATTAACCGTCGTGATCTGTGGGAAAAGGCGAAGAAAGACCAGCCGGAGTAATG
AAAGCTT

Bioconjugation of AG-20B-0014 with EEV12

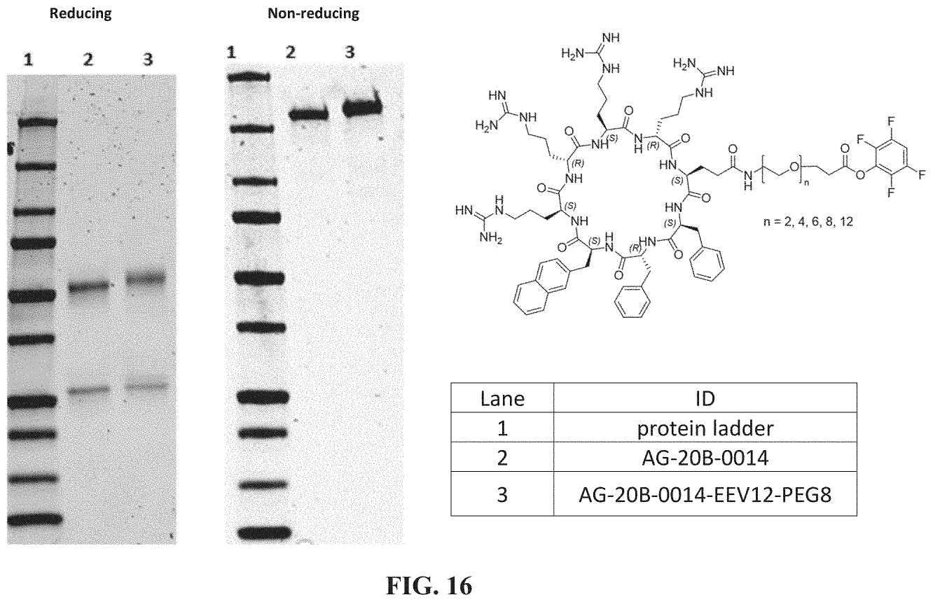

Various AG-20B-0014-EEV12 conjugates were prepared. AG-20B-0014 (available from AdipoGen, San Diego, Calif.) is an anti-NLRP3 mAb. To prepare the conjugates, AG-20B-0014 (0.85 mg/mL) was mixed with EEV12-PEG8-TFP (FIG. 16) in pH 7.4, phosphate buffered saline (1×), followed by the addition of 5% (v/v) of 7.5% sodium bicarbonate solution. The reaction was carried out at room temperature for 2 h before analyzed by SDS-PAGE to confirm the completion of bioconjugation (FIG. 16). Reaction was then quenched with glycine, and small molecules as well as extra peptides were removed by spin desalting.

### In Vitro Evaluation of AG-20B-0014-EEV12PEG8

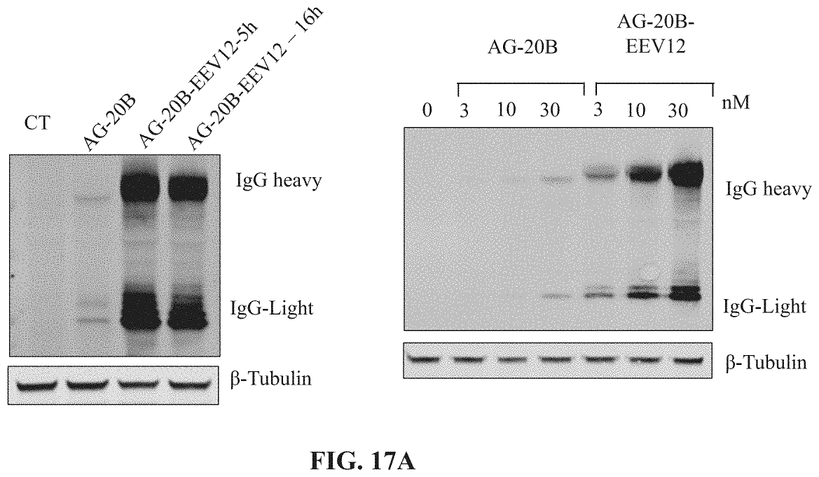

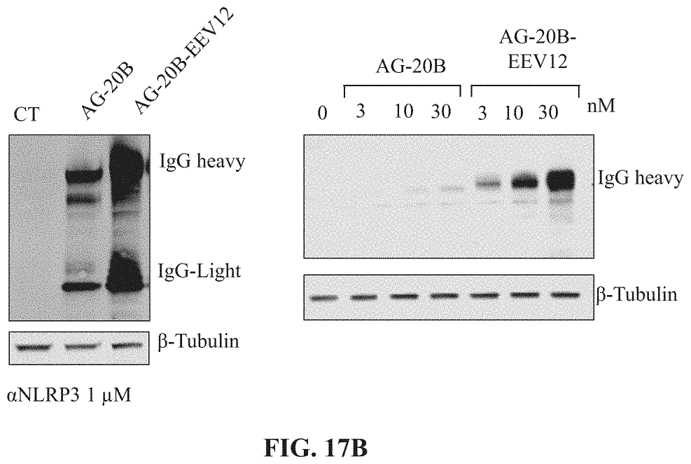

To study the EEV-mediated full-length IgG (AG-20B-EEV12) uptake in macrophages, Human CD14⁺ monocytes and THP1 cells were plated in 6-well tissue culture plates with 2×10⁶ cells per well and differentiated for 7 days into macrophages with 40 ng/ml recombinant MCSF and 2 days with 40 ng/ml PMA, respectively. The human monocytes derived macrophages were treated with different concentrations of anti-NLRP3 mouse IgG AG-20B or AG-20B-EEV12 conjugate. Samples were evaluated by Western Blot. The data indicate that EEV-conjugation significantly enhanced the cellular uptake of anti-human NLRP3 antibody in Human CD14⁺ monocytes (FIG. 17A) and THP1 derived macrophages (FIG. 17B).

### Example 15: Evaluation of Various ACS Targeting Degradation Constructs and Degradation Compounds

Various VHH^{CARD} sdAb degradation constructs and compounds were evaluated for targeting ACS proteins.

Target DNA sequence of human ASC and mouse ASC were optimized, synthesized, subcloned into vector pET-30a (+) with His+Flag tag for recombinant protein expression in E. coli using methods described herein above. Human ASC was purified in denatured state using Ni column then dialyzed to refold the protein. Mouse ASC was purified using Ni affinity chromatography. The protein purity and molecular weight were determined by standard SDS-PAGE along with Western blot confirmation using mouse anti-His mAb and rabbit anti-FLAG pAb or mouse anti-MBP mAb.

Human ACS:
(SEQ ID NO: 450)
MHHHHHHDYKDDDDK GRARDAILDALENLTAEELKKFKLKLLSVPLREG
YGRIPRGALLSMDALDLTDKLVSFYLETYGAELTANVLRDMGLQEMAGQL
QAATHQGSGAAPAGIQAPPQSAAKPGLHFIDQHRAALIARVTNVEWLLDA
LYGKVLTDEQYQAVRAEPTNPSKMRKLFSFTPAWNWTCKDLLLQALRESQ
SYLVEDLERS
Human ACS:
(SEQ ID NO: 451)
ATGCATCACCACCACCACCACGATTACAAGGATGATGATGATAAGGGACG
TGCGCGTGATGCGATCCTGGACGCGCTGGAGAACCTGACCGCGGAGGAAC
TGAAGAAATTCAAGCTGAAACTGCTGAGCGTGCCGCTGCGTGAAGGTTAT
GGCCGTATTCCGCGTGGTGCGCTGCTGAGCATGGATGCGCTGGATCTGAC
CGACAAGCTGGTGAGCTTTTACCTGGAGACCTATGGCGCGGAACTGACCG
CGAACGTTCTGCGTGATATGGGTCTGCAAGAGATGGCGGGTCAGCTGCAA
GCGGCGACCCACCAAGGTAGCGGTGCGGCGCCGGCGGGTATCCAAGCGCC
GCCGCAAAGCGCGGCGAAGCCGGGTCTGCACTTCATTGACCAGCACCGTG
CGGCGCTGATTGCGCGTGTGACCAACGTTGAATGGCTGCTGGATGCGCTG
TACGGCAAAGTGCTGACCGACGAGCAGTATCAAGCGGTTCGTGCGGAACC
GACCAACCCGAGCAAGATGCGTAAACTGTTCAGCTTTACCCCGGCGTGGA
ACTGGACCTGCAAAGATCTGCTGCTGCAGGCGCTGCGTGAGAGCCAAAGC
TACCTGGTTGAGGACCTGGAACGTAGCTAATGAAAGCTT
Mouse ACS:
(SEQ ID NO: 452)
KLGSGSSGSGENLYFQGMGRARDAILDALENLSGDELKKFKMKLLTVQLR
EGYGRIPRGALLQMDAIDLTDKLVSYYLESYGLELTMTVLRDMGLQELAE
QLQTTKEESGAVAAAASVPAQSTARTGHFVDQHRQALIARVTEVDGVLDA
LHGSVLTEGQYQAVRAETTSQDKMRKLFSFVPSWNLTCKDSLLQALKEIH
PYLVMDLEQS
Mouse ACS:
(SEQ ID NO: 449)
ATGCATCACCACCACCACCACGATTACAAGGATGATGATGATAAGACAAG
CGTGCGTTGCAAACTGGCGCAGTATCTGGAGGACCTGGAAGACGTTGATC
TGAAGAAATTCAAAATGCACCTGGAGGATTACCCGCCGGAAAAGGGTTGC
ATCCCGGTTCCGCGTGGCCAAATGGAAAAAGCGGACCACCTGGATCTGGC
GACCCTGATGATTGACTTCAACGGCGAGGAAAAGGCGTGGGCGATGGCGG
TGTGGATCTTTGCGGCGATTAACCGTCGTGATCTGTGGGAAAAGGCGAAG
AAAGACCAGCCGGAGTAATGAAAGCTT

Various VHH^{CARD} sdAb and VHH^{CARD} sdAb degradation constructs were designed and produced (Table 25).

TABLE 25
VHH^{CARD} sdAb and VHH^{CARD} sdAb degradation constructs

| | Protein | DNA | |
|---|---|---|---|
| | SEQ ID | SEQ ID | Mutations or |
| VHH^{CARD} fusions | NO: | NO: | Additional Amino Acids |
| VHH^{CARD}-Cys | | | ASC-CARD nanobody (VHH) |
| VHH^{CARD} | | | |
| VHH^{CARD}(LPETG) | | | |
| VHH^{CARD}-Fc-Cys | | | Fc (N297A), C-terminal Cys |
| VHH^{CARD}-Fc | | | |
| VHL₅₄₋₂₁₃-VHH^{CARD} | | | |
| VHH^{CARD}-Fc-_{239C} | | | Fc (N297A, S239C) |
| VHH^{CARD}-Mb-Cys | | | C-terminal Cys |
| VHH^{CARD}-Mb | | | |
| Cys-VHH^{CARD}-Mb- | | | N-terminal Cys, C-terminal |
| VHL₁₅₂₋₂₁₃ | | | VHL₁₅₂₋₂₁₃ |
| VHL₁₅₂₋₂₁₃- | | | C-terminal Cys , N-terminal |
| VHH^{CARD}-Mb-Cys | | | VHL₁₅₂₋₂₁₃ |
| VHL-VHH^{CARD} | | | |
| Cys-VHH^{CARD-} | | | N-terminal Cys, C-terminal |
| VHLpep | | | VHL peptide |
| VHLpep-VHH^{CARD-} | | | C-terminal Cys, N-terminal |
| Cys | | | VHL peptide |
| Cys-VHH^{CARD-} | | | N-terminal Cys, C-terminal |
| VHL₁₅₂₋₂₁₃ | | | SOCS1 peptide |
| VHL₁₅₂₋₂₁₃- | | | C-terminal Cys, N-terminal |
| VHH^{CARD}-Cys | | | SOCS1 peptide |
| VHL₅₄₋₂₁₃-VHH^{CARD} | | | |
| Cys-VHL₁₅₂₋₂₁₃- | | | N-terminal VHL & C-terminal |
| VHH^{CARD}-SOCS1pep | | | SOCS1pep |
| SOCS1pep-VHL₁₅₂₋₂₁₃- | | | N-terminal VHL₁₅₂₋₂₁₃ |
| VHH^{CARD}-Cys | | | |
| Cys-VHH^{CARD-} | | | C-terminal TRIM21₁₋₂₇₇ |
| TRIM21₁₋₂₇₇ | | | |
| TRIM21₁₋₂₇₇- | | | N-terminal TRIM21₁₋₂₇₇ |
| VHH^{CARD}-Cys | | | |
| Cys-VHH^{CARD-} | | | C-terminal SOCS1pep |
| SOCS1pep | | | |
| SOCS1pep- | | | N-terminal SOCS1pep |
| VHH^{CARD}-Cys | | | |
| VHL₁₅₂₋₂₁₃- | | | N-terminal VHL & C-terminal |
| VHH^{CARD}- | | | TRIM21₁₋₂₇₇ |
| TRIM21₁₋₂₇₇-CyS | | | |

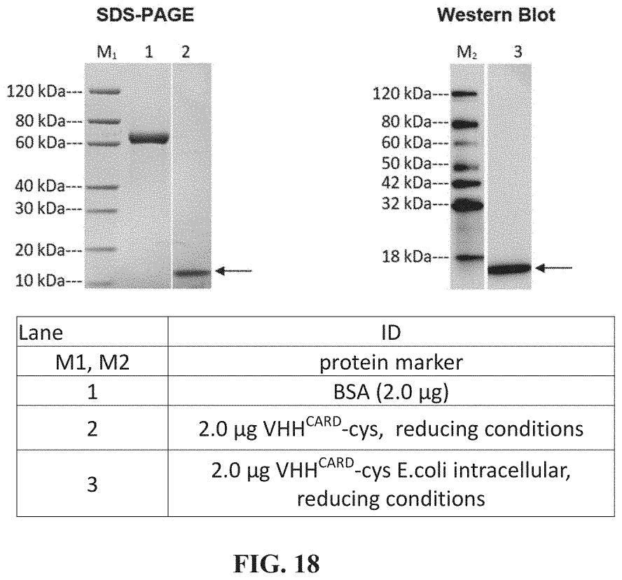

Target DNA sequences of VHH^{CARD}-Cys, VHH^{CARD}, and VHH^{CARD}-LPETG were optimized, synthesized, subcloned into vector pET-28a with His tag and TEV protease cleavage site for recombinant protein expression in E. coli. Protein was produced using methods similar to those described herein above. Cells were lysed and purified using Ni affinity chromatography. Protein purity and molecular weight were determined by standard SDS-PAGE along with Western blot confirmation using mouse anti-His mAb (FIG. 18).

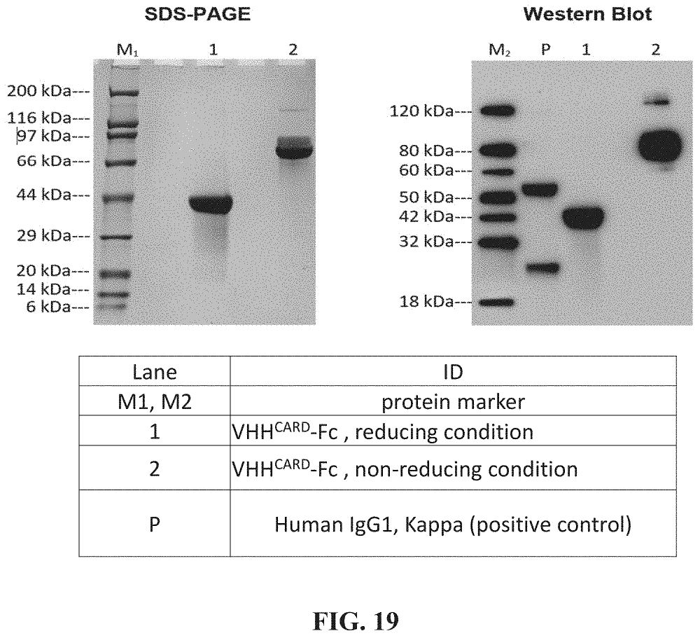

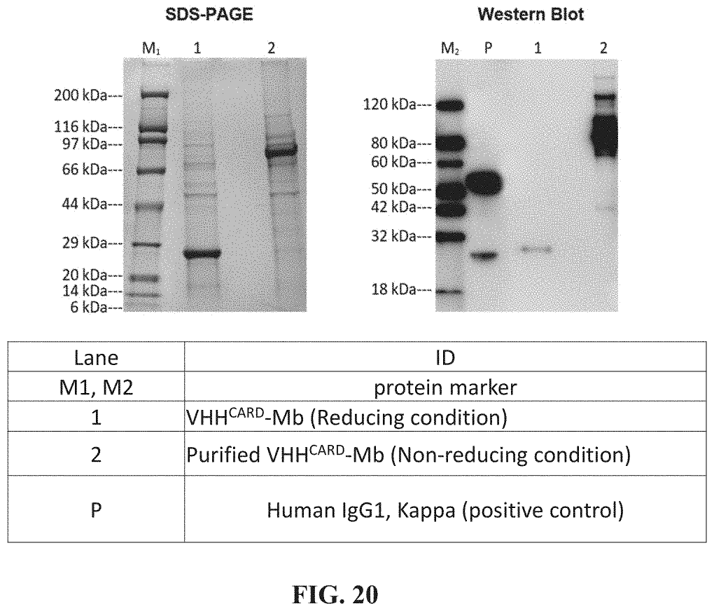

VHH^{CARD}-c, VHL-VHH^{CARD}, VHL₅₄₋₂₁₃ VHH^{CARD}, and VHH^{CARD}-Mb fusion sequence was designed, optimized, synthesized, subcloned into pcDNA3.4 vector, expressed using secreted mammalian expression, and purified using methods similar to those described in Example 1. Purified protein was analyzed by SDS-PAGE, Western blot (primary antibody was goat anti-human JgG-HIRP) and HPLC analysis to determine the molecular weight and purity (FIG. 19 and FIG. 20).

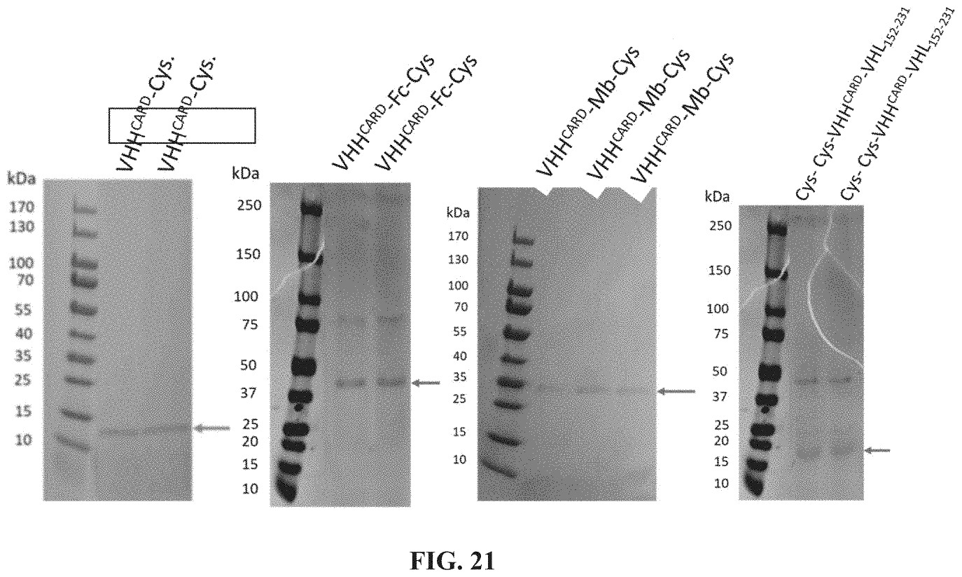

Various VHH^{CARD}-cys, VHH^{CARD}-FC-cys, VHH^{CARD}-Fc(S239A), VHH^{CARD}-Mb-cys, Cys-VHH^{CARD}-Mb-VHL₁₅₂₋₂₁₃, VHL₁₅₂₋₂₁₃-VHH_{CARD}-Mb-cys, Cys-VHH^{CARD}-VHLpep, VHLpep-VHH^{CARD}-cys, Cys-VHH^{CARD}-VHL₁₅₂₋₂₁₃, VHL₁₅₂₋₂₁₃-VHH^{CARD}, Cys-VHL₁₅₂₋₂₁₃-VHH^{CARD}-SoCS1pep, SOCS1pep-VHL₁₅₂₋₂₁₃-VHH^{CARD}-cys, cys-VHH^{CARD}-TRIM21₁₋₂₇₇, TRIM21₁₋₂₇₇-VHH^{CARD}-Cys, cys-VHH^{CARD}-SOCS1pep, VHH^{CARD}-SOCS1pep-cys, and VHL fusion were also expressed in P. pastoris and purified using methods similar those described in Example 1 for small, medium, and large batches. SDS-PAGE was used to analyze the expression of VHH^{CARD} with C-terminal Cys residue (Examples are shown in FIG. 21).

### Example 16: Evaluation of Various VHH^{CARD} Degradation Constructs and Compounds

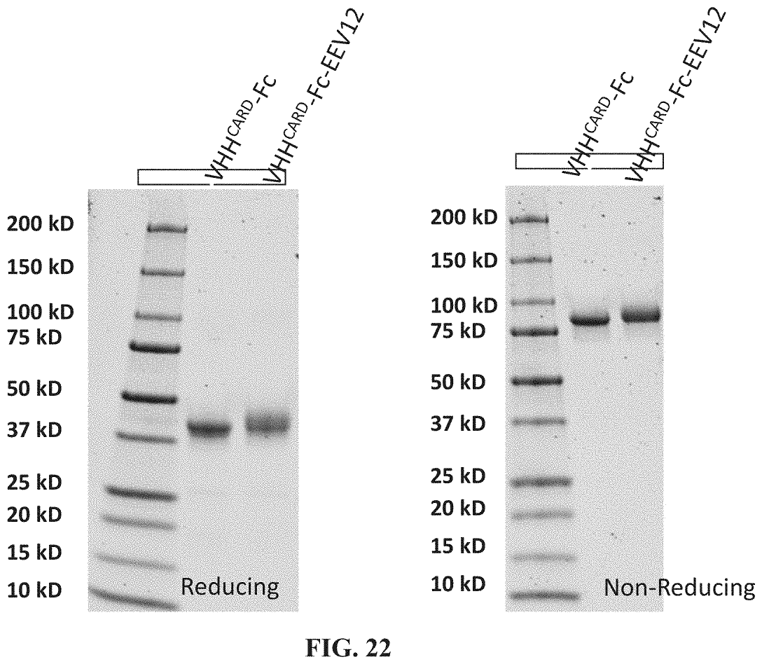

VHH^{CARD}-Fc was conjugated with EEV12-PEG8-TFP conjugate through primary amine conjugation. Briefly, VHH^{CARD}-Fc (from Example 13) was mixed with EEV12-PEG₈-TFP (169 μM) in pH 7.4, phosphate buffered saline (1×). The reaction was carried out at room temperature for 2 h before analyzed by SDS-PAGE to confirm the completion of bioconjugation (FIG. 22). Reaction was then quenched with glycine, and small molecules as well as extra peptides were removed via spin desalting.

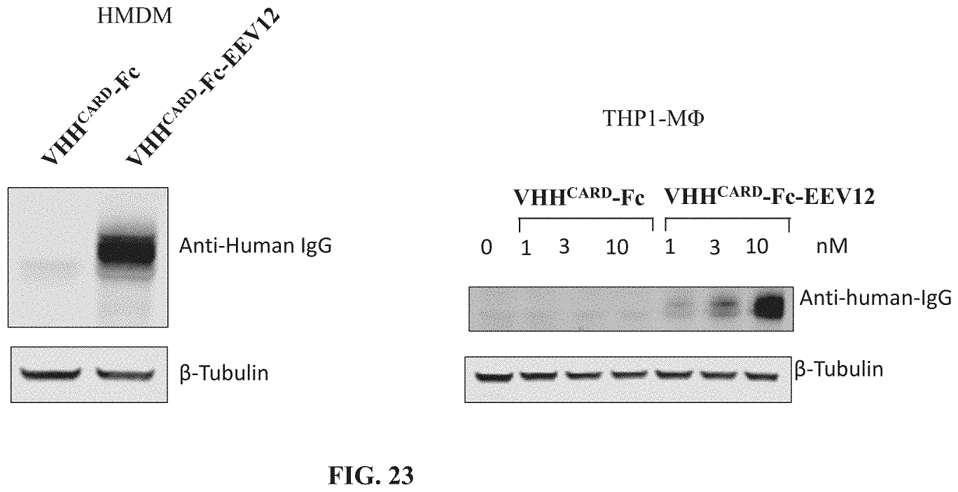

To study the EEV12-mediated VHH^{CARD} uptake in macrophages, human CD14⁺ monocytes and THP1 cells were treated with VHH^{CARD}-Fc-EEV12 or VHH^{CARD}-Fc using methods similar to those described in Example 2. The data demonstrate that EEV12-conjugation significantly enhanced the cellular uptake of VHH^{CARD}-Fc in human derived macrophages (FIG. 23).

### Example 17: Methods for Generating of VHH sdAbs Against NLRP3 Proteins

Single domain antibodies may be developed for binding NLRP3 proteins using methods similar to those described in Example 5.

### Example 18: Methods for Evaluating the PK, TK, and Biodistribution of Degradation Compounds Against NLRP3 Proteins in Rodent Species

Degradation compounds targeting NLRP3 proteins may be evaluated using methods similar to Example 9.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A degradation compound comprising:
a cyclic cell penetrating peptide (cCPP); and
a degradation construct comprising a degradation moiety and a targeting moiety.

2. The degradation compound of claim 1, wherein the cCPP is not selected from:
(SEQ ID NO: 302)
FΦRRRQ,
(SEQ ID NO: 303)
FΦRRRC,
(SEQ ID NO: 304)
FΦRRRU,
(SEQ ID NO: 305)
RRRΦFQ,
(SEQ ID NO: 306)
RRRRΦF,
(SEQ ID NO: 307)
FΦRRRR,
(SEQ ID NO: 301)
FϕrRrRq, FϕrRrRQ, FΦRRRRQ,
(SEQ ID NO: 308)
fΦrRrQ, RRFRΦRQ,
(SEQ ID NO: 309)
FRRRRΦQ,
(SEQ ID NO: 310)
rRFRΦRQ, RRΦFRRQ,
(SEQ ID NO: 311)
CRRRRFWQ,
(SEQ ID NO: 312)
FfΦRrRrQ, FFΦRRRRQ,
(SEQ ID NO: 313)
RFRFRΦRQ,
(SEQ ID NO: 314)
URRRRFWQ,
(SEQ ID NO: 311)
CRRRRFWQ,
(SEQ ID NO: 315)
FΦRRRRQK,
(SEQ ID NO: 316)
FPRRRRQC,
(SEQ ID NO: 317)
fΦRrRrRQ, FΦRRRRRQ,
(SEQ ID NO: 318)
RRRRΦFDΩC,
(SEQ ID NO: 319)
FΦRRR,
(SEQ ID NO: 320)
FWRRR,
(SEQ ID NO: 321)
RRRΦF,
and
(SEQ ID NO: 322)
RRRWF,
where F = L-naphthylalanine; f = D-naphthylala-
nine; Ω = L-norleucine.

3. The degradation compound of claim 1, further comprising an exocyclic peptide.

4. The degradation compound of claim 3, wherein the exocyclic peptide comprises one of the following sequences:
(SEQ ID NO: 1)
KK, KR, RR, HH, HK, HR, RH, KKK, KGK, KBK, KBR,
KRK, KRR, RKK, RRR, KKH, KHK, HKK, HRR, HRH, HHR,
HBH, HHH, HHHH,
(SEQ ID NO: 2)
KHKK,
(SEQ ID NO: 3)
KKHK,
(SEQ ID NO: 4)
KKKH,
(SEQ ID NO: 5)
KHKH,
(SEQ ID NO: 6)
HKHK,
(SEQ ID NO: 7)
KKKK,
(SEQ ID NO: 8)
KKRK,
(SEQ ID NO: 9)
KRKK,
(SEQ ID NO: 10)
KRRK,
(SEQ ID NO: 11)
RKKR,
(SEQ ID NO: 12)
RRRR,
(SEQ ID NO: 13)
KGKK,
(SEQ ID NO: 14)
KKGK,
(SEQ ID NO: 15)
HBHBH,
(SEQ ID NO: 16)
HBKBH,
(SEQ ID NO: 17)
RRRRR,
(SEQ ID NO: 18)
KKKKK,
(SEQ ID NO: 19)
KKKRK,
(SEQ ID NO: 20)
RKKKK,
(SEQ ID NO: 21)
KRKKK,
(SEQ ID NO: 22)
KKRKK,
(SEQ ID NO: 23)
KKKKR,
(SEQ ID NO: 24)
KBKBK,
(SEQ ID NO: 25)
RKKKKG,
(SEQ ID NO: 26)
KRKKKG,
(SEQ ID NO: 27)
KKRKKG,
(SEQ ID NO: 28)
KKKKRG,
(SEQ ID NO: 29)
RKKKKB,
(SEQ ID NO: 30)
KRKKKB,
(SEQ ID NO: 31)
KKRKKB,
(SEQ ID NO: 32)
KKKKRB,
(SEQ ID NO: 33)
KKKRKV,
(SEQ ID NO: 34)
RRRRRR,
(SEQ ID NO: 35)
HHHHHH,
(SEQ ID NO: 36)
RHRHRH,
(SEQ ID NO: 37)
HRHRHR,
(SEQ ID NO: 38)
KRKRKR,
(SEQ ID NO: 39)
RKRKRK,
(SEQ ID NO: 40)
RBRBRB,
(SEQ ID NO: 41)
KBKBKB,
(SEQ ID NO: 42)
PKKKRKV,
(SEQ ID NO: 43)
PGKKRKV,
(SEQ ID NO: 44)
PKGKRKV,
(SEQ ID NO: 45)
PKKGRKV,
(SEQ ID NO: 46)
PKKKGKV,
(SEQ ID NO: 47)
PKKKRGV,
(SEQ ID NO: 48)
PKKKRKG,
(SEQ ID NO: 280)
NLSKRPAAIKKAGQAKKKK,
(SEQ ID NO: 281)
PAAKRVKLD,
(SEQ ID NO: 282)
RQRRNELKRSF,
(SEQ ID NO: 283)
RMRKFKNKGKDTAELRRRRVEVSVELR,
(SEQ ID NO: 284)
KAKKDEQILKRRNV,
(SEQ ID NO: 285)
VSRKRPRP,
(SEQ ID NO: 286)
PPKKARED,
(SEQ ID NO: 287)
PQPKKKPL,
(SEQ ID NO: 288)
SALIKKKKKMAP,
(SEQ ID NO: 289)
DRLRR,
(SEQ ID NO: 290)
PKQKKRK,
(SEQ ID NO: 291)
RKLKKKIKKL,
(SEQ ID NO: 292)
REKKKFLKRR,
(SEQ ID NO: 293)
KRKGDEVDGVDEVAKKKSKK
or
(SEQ ID NO: 294)
RKCLQAGMNLEARKTKK,
wherein B is beta-alanine.

5. The degradation compound of claim 1, wherein the cCPP is of Formula (A): 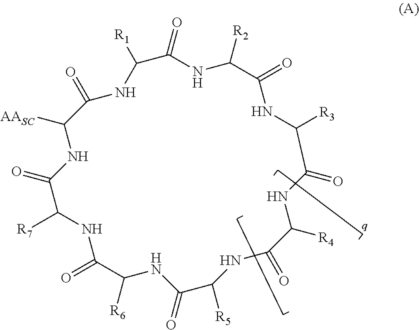
or a protonated form thereof, wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl)alanine;
AA_{SC} is an amino acid side chain; and
q is 1, 2, 3 or 4.

6. The degradation compound of claim 1, wherein the cCPP is of Formula (I): 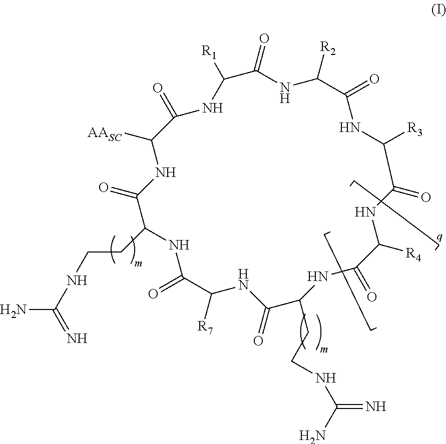
or a protonated form or salt thereof,
wherein each m is independently an integer from 0-3.

7. The degradation compound of claim 6, wherein R₁, R₂, and R₃ are independently H or a side chain comprising an aryl group.

8. The degradation compound of claim 6, wherein the cCPP is of Formula (I-1), (I-2), (I-3), (I-4), (I-5), or (I-6): 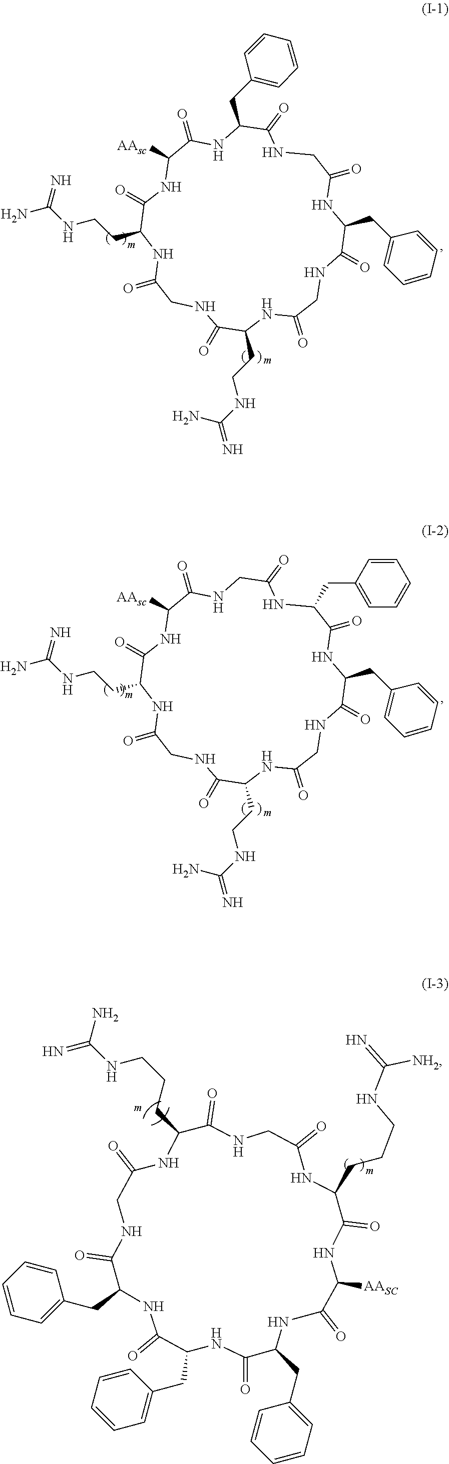 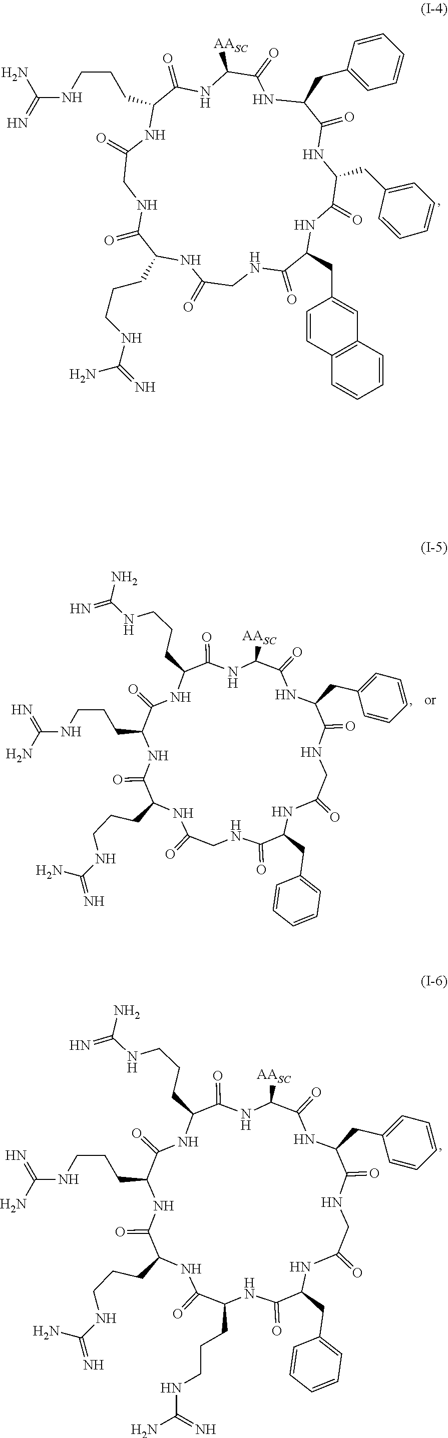
a protonated form or salt of Formula (I-1), (I-2), (I-3), (I-4), (I-5), or (I-6).

9. The degradation compound of claim 1, wherein the cCPP is of Formula (II): 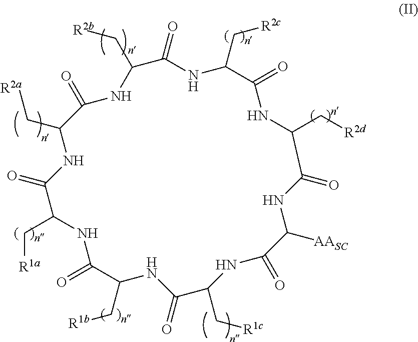
wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is 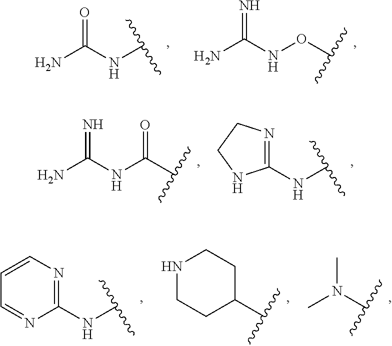
or a protonated form or salt thereof,
at least one of R^{2a}, R^{2b}, R^{2c} and R^{2d} is guanidine or a protonated form or salt thereof,
each n″ is independently an integer from 0 to 5;
each n′ is independently an integer from 0 to 3; and
if n′ is 0 then R^{2a}, R^{2b}, R^{2c} or R^{2d} is absent.

10. The degradation compound of claim 9, wherein the cCPP is of Formula (II-1):

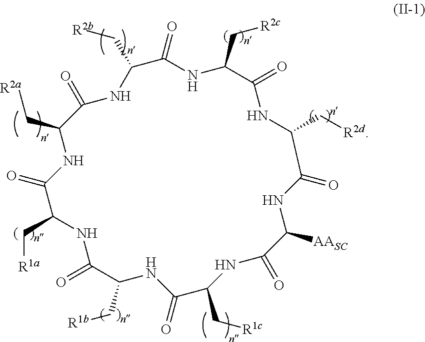

11. The degradation compound of claim 9, wherein the cCPP is of Formula (IIa):

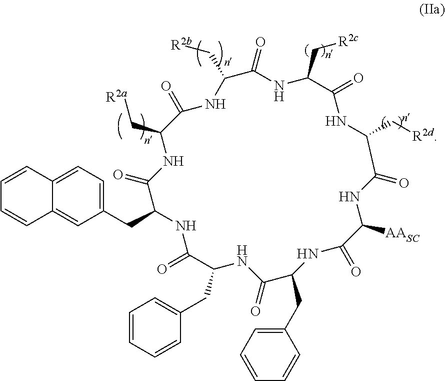

12. The degradation compound of claim 9, wherein the cyclic peptide is of Formula (IIb):

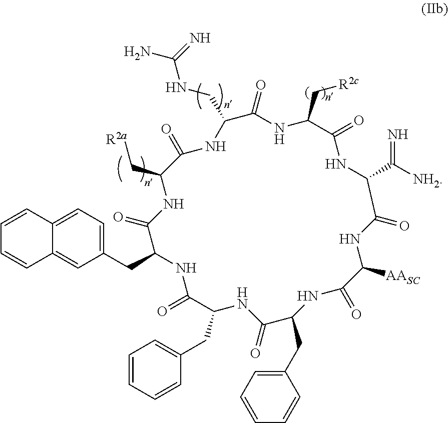

13. The degradation compound of claim 9, wherein the cCPP is of Formula (IIc): 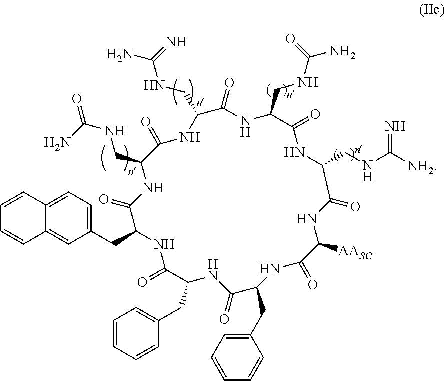
or a protonated form or salt thereof.

14. The degradation compound of claim 1, wherein the cCPP has the structure: 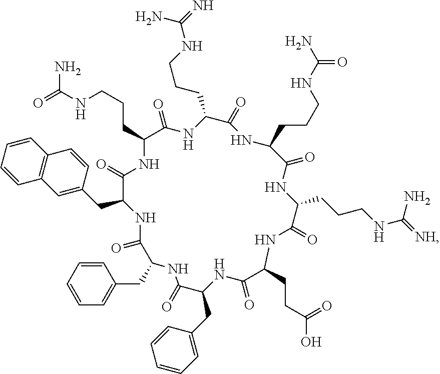 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]
or a protonated form or salt thereof, wherein at least one atom of an amino acid side chain is replaced by the degradation construct or a linker or at least one lone pair forms a bond to the degradation construct or the linker.

15. The degradation compound of claim 1, wherein the cCPP has the structure: 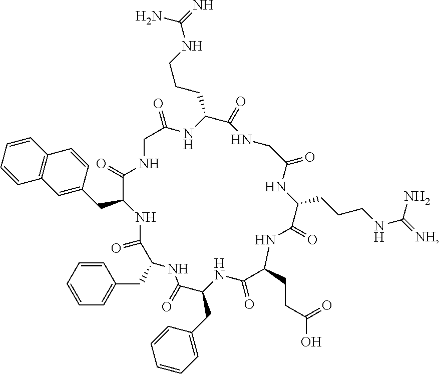 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]
or a protonated form or salt thereof, wherein at least one atom of an amino acid side chain is replaced by the degradation construct or a linker or at least one lone pair forms a bond to the degradation construct or the linker.

16. The degradation compound of claim 1, wherein the degradation moiety is a ubiquitin ligase enzyme or is a peptide that interacts with an endogenous ubiquitin ligase enzyme.

17. The degradation compound of claim 1, wherein the targeting moiety is a peptide, an antibody or antigen-binding fragment thereof, a Designed Ankyrin Repeat Protein (DARPin), or a fibronectin-based scaffold protein.

18. The degradation compound of claim 1, wherein targeting moiety specifically binds β-catenin, NALP3, KRAS, MDM2, EGFR, ASC, or IRF-5, or an infectious agent.

19. A method for degrading a target protein within a cell, the method comprising:
contacting the exterior of the cell with a degrader compound, the degrader compound comprising:
a cyclic cell penetrating peptide (cCPP); and
a degradation construct comprising a degradation moiety and a targeting moiety,
wherein the targeting moiety binds the target protein.

20. The method of claim 19, wherein the degrader compound further comprises an exocyclic peptide.

21. The method of claim 19, wherein contacting the exterior of the cell with the degrader compound comprises administering a pharmaceutical composition to a subject comprising the cell.

22. The method of claim 21, wherein the subject is suffering from a disease, and wherein administration of the pharmaceutical composition to the subject treats the disease.
